(19) United States
(12) Patent Application Publication
(10) Pub. No.: US 2012/0264735 A1
(43) Pub. Date: Oct. 18, 2012

(54) TYROSINE KINASE INHIBITORS

(76) Inventors: Jonathan Young, Southborough, MA (US); Barbara Czako, Houston, TX (US); Michael Altman, Needham, MA (US); David Guerin, Natick, MA (US); Michelle Martinez, Medford, MA (US); Alexey Rivkin, Emeryville, CA (US); Kevin Wilson, Newton, MA (US); Kathryn Lipford, Boston, MA (US); Catherine White, Newton Center, MA (US); Laura Surdi, Franklin, MA (US); Stephanie Chichetti, Bay Shore, NY (US); Matthew H. Daniels, Cambridge, MA (US); Sean P. Ahearn, Somerville, MA (US); Danielle Falcone, Brookline, MA (US); Ekundayo Osimboni, Ashland, MA (US)

(21) Appl. No.: 13/517,321

(22) PCT Filed: Dec. 14, 2010
(86) PCT No.: PCT/US2010/060192

§ 371 Date: Jun. 20, 2012

Related U.S. Application Data
(60) Provisional application No. 61/288,610, filed on Dec. 21, 2009.

(51) Int. Cl. A61K 31/501; A61K 31/506; C07D 413/12; A61P 35/00; C07D 413/14; C07D 417/14; A61K 31/541; C07D 403/04; A61K 31/5377

(52) U.S. Cl. 514/210.18; 544/238; 514/252.05; 514/252.02; 544/114; 514/236.5; 544/122; 544/58.6; 514/227.8; 544/58.2

(57) ABSTRACT

The present invention relates to pyridazin-4(1H)-one derivatives, that are useful for treating cellular proliferative diseases, for treating disorders associated with MET activity, and for inhibiting the receptor tyrosine kinase MET. The invention also related to compositions which comprise these compounds, and methods of using them to treat cancer in mammals.

### BACKGROUND OF THE INVENTION

This invention relates to pyridazin-4(1H)-one compounds that are inhibitors of tyrosine kinases, in particular the receptor tyrosine kinase MET, and are useful in the treatment of cellular proliferative diseases, for example cancer, hyperplasias, restenosis, cardiac hypertrophy, immune-disorders and inflammation.

Studies on signal transduction pathways have generated various promising molecular targets for therapeutic inhibition in cancer therapy. Receptor tyrosine kinases (RTK) represent an important class of such therapeutic targets. Recently, members of the MET proto-oncogene family, a subfamily of receptor tyrosine kinases, have drawn special attention to the association between invasion and metastasis. The MET family, including MET (also referred to as c-Met) and RON receptors, can function as oncogenes like most tyrosine kinases. MET has been shown to be overexpressed and/or mutated in a variety of malignancies. A number of MET activating mutations, many of which are located in the tyrosine kinase domain, have been detected in various solid tumors and have been implicated in invasion and metastasis of tumor cells.

The c-Met proto-oncogene encodes the MET receptor tyrosine kinase. The MET receptor is an approximately 190 kDa glycosylated dimeric complex composed of a 50 kDa alpha chain disulfide-linked to a 145 kDa beta chain. The alpha chain is found extracellularly while the beta chain contains extracellular, transmembrane and cytosolic domains. MET is synthesized as a precursor and is proteolytically cleaved to yield mature alpha and beta subunits. It displays structural similarities to semaphoring and plexins, a ligand-receptor family that is involved in cell-cell interaction.

The natural ligand for MET is hepatocyte growth factor (HGF), a disulfide linked heterodimeric member of the scatter factor family that is produced predominantly by mesenchymal cells and acts primarily on MET-expressing epithelial and endothelial cells in an endocrine and/or paraendocrine fashion. HGF has some homology to plasminogen.

It is known that stimulation of MET via hepatocyte growth factor (also known as scatter factor, HGF/SF) results in a plethora of biological and biochemical effects in the cell. Activation of c-Met signaling can lead to a wide array of cellular responses including proliferation, survival, angiogenesis, wound healing, tissue regeneration, scattering, motility, invasion and branching morphogenesis. HGF/MET signaling also plays a major role in the invasive growth that is found in most tissues, including cartilage, bone, blood vessels, and neurons.

Various c-Met mutations have been well described in multiple solid tumors and some hematologic malignancies. The prototypic c-Met mutation examples are seen in hereditary and sporadic human papillary renal carcinoma (Schmidt, L. et al., Nat. Tenet. 1997, 16, 68-73; Jeffers, M. et al., Proc. Nat. Acad. Sci. 1997, 94, 11445-11500). Other reported examples of c-Met mutations include ovarian cancer, childhood hepatocellular carcinoma, metastatic head and neck squamous cell carcinomas and gastric cancers. HGF/MET has been shown to inhibit anoikis, suspension-induced programmed cell death (apoptosis), in head and neck squamous cell carcinoma cells.

MET signaling is implicated in various cancers, especially renal. The nexus between MET and colorectal cancer has also been established. Analysis of c-Met expression during colorectal cancer progression showed that 50% of the carcinoma specimens analyzed expressed 5-50-fold higher levels of MET mRNA transcripts and protein versus the adjacent normal colonic mucosa. In addition when compared to the primary tumor, 70% of colorectal cancer liver metastasis showed MET overexpression.

MET is also implicated in glioblastoma. High-grade malignant gliomas are the most common cancers of the central nervous system. Despite treatment with surgical resection, radiation therapy, and chemotherapy, the mean overall survival is <1.5 years, and few patients survive for >3 years. Human malignant gliomas frequently express both HUE and MET, which can establish an autocrine loop of biological significance. Glioma MET expression correlates with glioma grade, and an analysis of human tumor specimens showed that malignant gliomas have a 7-fold higher HGF content than low-grade gliomas. Multiple studies have demonstrated that human gliomas frequently co-express HGF and MET and that high levels of expression are associated with malignant progression. It was further shown that HGF-MET is able to activate Akt and protect glioma cell lines from apoptotic death, both in vitro and in vivo.

RON shares a similar structure, biochemical features, and biological properties with MET. Studies have shown RON overexpression in a significant fraction of breast carcinomas and colorectal adenocarcinomas, but not in normal breast epithelia or benign lesions. Cross-linking experiments have shown that RON and MET form a non-covalent complex on the cell surface and cooperate in intracellular signaling. RON and MET genes are significantly co-expressed in ovarian cancer cell motility and invasiveness. This suggests that co-expression of these two related receptors might confer a selective advantage to ovarian carcinoma cells during either tumor onset or progression.

A number of reviews on MET and its function as an oncogene have recently been published: Cancer and Metastasis Review 22:309-325 (2003); Nature Reviews/Molecular Cell Biology 4:915-925 (2003); Nature Reviews/Cancer 2:289-300 (2002).

Since dysregulation of the HGF/MET signaling has been implicated as a factor in tumorgenesis and disease progression in many tumors, different strategies for therapeutic inhibition of this important RTK molecule should be investigated. Specific small molecule inhibitors against HGF/MET signaling and against RON/MET signaling have important therapeutic value for the treatment of cancers in which Met activity contributes to the invasive/metastatic phenotype.

### SUMMARY OF THE INVENTION

The present invention relates to pyridazin-4(1H)-one derivatives, that are useful for treating cellular proliferative diseases, for treating disorders associated with MET activity, and for inhibiting the receptor tyrosine kinase MET. The compounds of the invention may be illustrated by the Formula I:

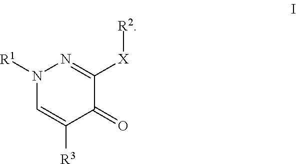

### DETAILED DESCRIPTION OF THE INVENTION

The compounds of this invention are useful in the inhibition of tyrosine kinses, in particular the receptor tyrosine kinase MET, and are illustrated by a compound of the formula:

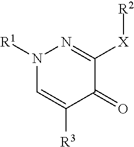

wherein X is O, S or CR⁴R^{4′};
R¹ is heteroaryl or aryl, wherein said heteroaryl and aryl groups are optionally substituted with one to three groups independently selected from the group consisting of halo, cyano, C₁₋₆ alkyl, (C₁₋₆ alkyl)R⁷, OR⁹, heterocyclyl(R⁷), aryl and heteroaryl(R⁵);
R² is heteroaryl or phenyl, wherein said heteroaryl group is optionally substituted with oxo, C₁₋₆ alkyl, NH(C═O)OR⁹ or OR⁹; and wherein said phenyl group is optionally substituted with one to two substituents independently selected from the group consisting of:
(1) halo,
(2) hydroxyl,
(3) cyano,
(4) heterocyclyl,
(5) heteroaryl, which is optionally substituted with one to two substituents independently selected from the group consisting of (C═O)OR⁹, NR⁵R⁹, NH(C═O)OR⁹, NH(C═O)R⁹, (C═O)NHR⁹, OR⁹ and R⁹,
(6) NH(C═O)OR⁹,
(7) NH(C═O)R⁹,
(8) NH(C═O)NHR⁹,
(9) (C═O)OR⁵, and
(10) C₁₋₃ alkyl(C═O)NHR⁵;
   R³ is hydrogen, halo or C₁₋₃ alkyl;
   R⁴ is hydrogen, halo or C₁₋₆ alkyl, wherein said alkyl is optionally substituted with hydroxyl or cyano;
   R^{4′} is hydrogen, halo or C₁₋₆ alkyl, wherein said alkyl is optionally substituted with hydroxyl or cyano;
   R⁵ is hydrogen or C₁₋₆ alkyl, wherein said alkyl is optionally substituted with hydroxyl;
   R⁶ is hydrogen or C₁₋₆ alkyl, wherein said alkyl is optionally substituted with hydroxyl;
   R⁷ is hydrogen, hydroxyl, C₁₋₆ alkyl, C₁₋₆ haloalkyl, heterocyclyl, OR⁹, heteroaryl(OR⁹), (C═O)R⁵, (C═O)OR⁵, (C═O)NR⁵R⁶, (C═O)heterocyclyl, (C═O)N(R⁵)heterocyclyl or NR⁵R⁶;
   R⁸ is hydrogen, halo, cyano, hydroxyl, C₁₋₆ alkyl, (C═O)NR⁵R⁶ or NR⁵R⁶;
   R⁹ is hydrogen, halo, C₁₋₆ alkyl, C₂₋₆ alkenyl, (C₂₋₆ alkenyl)OR⁵, (C₂₋₆ alkenyl)NR⁵R⁶, C₃₋₆ cycloalkyl, C₃₋₆ cycloalkyl(OR⁵), heterocyclyl (which is optionally substituted with one or two R⁸) or heteroaryl(R⁸), wherein said alkyl is optionally substituted with one to, four groups independently selected from the group consisting of halo, hydroxyl, cyano, OR¹⁰, (C═O)NR⁵R⁶, (C═O)OR⁵, SO₂CH₃, NR⁵R¹⁰, C₃₋₈ cycloalkyl, heterocyclyl(which is optionally substituted with one or two R¹⁰), heteroaryl(R¹⁰, (aryl)OR⁵, phenyl and phenyl(O-benzyl);
   R¹⁰ is hydrogen, halo, oxo, C₁₋₆ alkyl, (C₁₋₆ alkyl)OR⁵, C₁₋₆ haloalkyl, C₃₋₈ cycloalkyl, aryl and (C═O)OR⁵;
   or a pharmaceutically acceptable salt thereof.

In a class of the invention, X is CR⁴R^{4′}.

In a class of the invention, R¹ is heteroaryl, wherein said heteroaryl group is optionally substituted with one to three groups independently selected from the group consisting of halo, cyano, C₁₋₆ alkyl, (C₁₋₆ alkyl)R⁷, OR⁹, heterocyclyl(R⁷), aryl and heteroaryl(R⁵). In a subclass of the invention, R¹ is heteroaryl, wherein said heteroaryl group is optionally substituted with C₁₋₆ alkyl.

In a class of the invention, R² is phenyl, wherein said phenyl group is optionally substituted with one to two substituents independently selected from the group consisting of
(1) halo,
(2) hydroxyl,
(3) cyano,
(4) heterocyclyl,
(5) heteroaryl, which is optionally substituted with one to two substituents independently selected from the group consisting of (C═O)OR⁹, NR⁵R⁹, NH(C═O)OR⁹, NH(C═O)R⁹, (C═O)NHR⁹, OR⁹ and R⁹,
(6) NH(C═O)OR⁹,
(7) NH(C═O)R⁹,
(8) NH(C═O)NHR⁹,
(9) (C═O)OR⁵, and
(10) C₁₋₃ alkyl(C═O)NHR⁵.
   In a subclass of the invention, R² is phenyl, wherein said phenyl group is substituted with heteroaryl, which is optionally substituted with OR⁹ or R⁹. In another subclass of the invention, R² is phenyl, wherein said phenyl group is substituted with NH(C═O)OR9.

In a class of the invention, R³ is hydrogen or fluoro. In a subclass of the invention, R3 is hydrogen.

In a class of the invention, R4 is hydrogen.

In a class of the invention, R4′ is hydrogen.

Specific examples of the compounds of the instant invention include:
ethyl(3-{[1-(1-methyl-1H-pyrazol-4-yl)-4-oxo-1,4-dihydropyridazin-3-yl]methyl}phenyl)carbamate;
ethyl(3-{[4-oxo-1-(3,4,5-trifluorophenyl)-1,4-dihydropyridazin-3-yl]methyl}phenyl)carbamate;
3-[3-(5-ethoxypyrimidin-2-yl)benzyl]-1-(1-methyl-1H-pyrazol-4-yl)pyridazin-4(1H)-one;
3-{3-[5-(2-methoxyethoxy)pyrimidin-2-yl]benzyl}-1-(1-methyl-1H-pyrazol-4-yl)pyridazin-4(1H)-one;
3-[3-(5-methoxypyrimidin-2-yl)benzyl]-1-(1-methyl-1H-pyrazol-4-yl)pyridazin-4(1H)-one;
3-fluoro-5-[3-{3-[5-(2-hydroxy-2-methylpropoxy)pyrimidin-2-yl]benzyl}-4-oxopyridazin-1(4H)-yl]benzonitrile;
2-methylpropyl (3-{[4-oxo-1-(3,4,5-trifluorophenyl)-1,4-dihydropyridazin-3-yl]methyl}phenyl)carbamate;
   propyl (3-{[4-oxo-1-(3,4,5-trifluorophenyl)-1,4-dihydropyridazin-3-yl]methyl}phenyl)carbamate;
3-[3-(5-methoxypyrimidin-2-yl)benzyl]-1-(3,4,5-trifluorophenyl)pyridazin-4(1H)-one;
3-([1,2,4]triazolo[1,5-a]pyridin-6-ylmethyl)-1-(3,4,5-trifluorophenyl)pyridazin-4(1H-one;
ethyl(3-{[1-(3,5-difluorophenyl)-4-oxo-1,4-dihydropyridazin-3-yl]methyl}phenyl)carbamate;
propyl (3-{[1-(3-bromophenyl)-4-oxo-1,4-dihydropyridazin-3-yl]methyl}phenyl)carbamate;
ethyl(3-{[1-(3-bromophenyl)-4-oxo-1,4-dihydropyridazin-3-yl]methyl}phenyl)carbamate;
propyl (3-{[1-(4-bromophenyl)-4-oxo-1,4-dihydropyridazin-3-yl]methyl}phenyl)carbamate;
methyl 2-(3-{[1-(1-methyl-1H-pyrazol-4-yl)-4-oxo-1,4-dihydropyridazin-3-yl]methyl}phenyl)pyrimidine-5-carboxylate;
propyl (3-{[1-(4-bromo-3,5-difluorophenyl)-4-oxo-1,4-dihydropyridazin-3-yl]methyl}phenyl)carbamate;
2-methylpropyl (3-{[1-(3,5-difluorophenyl)-4-oxo-1,4-dihydropyridazin-3-yl]methyl}phenyl)carbamate;
ethyl(3-{[1-(4-bromophenyl)-4-oxo-1,4-dihydropyridazin-3-yl]methyl}phenyl)carbamate;
1-(1-methyl-1H-pyrazol-4-yl)-3-[3-(5-methyl-1,3-thiazol-2-yl)benzyl]pyridazin-4(1H)-one;
1-(1-methyl-1H-pyrazol-4-yl)-3-[3-(1,3-thiazol-2-yl)benzyl]pyridazin-4(1H)-one;
1-(1-methyl-1H-pyrazol-4-yl)-3-[3-(1H-pyrazol-t-yl)benzyl]pyridazin-4(1H)-one;
2-morpholin-4-ylethyl(3-{[1-(3,4-difluorophenyl)-4-oxo-1,4-dihydropyridazin-3-yl]methyl}phenyl)carbamate;
5-{[1-(1-methyl-1H-pyrazol-4-yl)-4-oxo-1,4-dihydropyridazin-3-yl]methyl}-1,3-dihydro-2H-benzimidazol-2-one;
5-{[1-(1-methyl-1H-pyrazol-4-yl)-4-oxa-1,4-dihydropyridazin-3-yl]methyl}-1,3-benzoxazol-2(3H)-one;
ethyl(3-{[1-(1-methyl-1H-pyrazol-3-yl)-4-oxo-1,4-dihydropyridazin-3-yl]methyl}phenyl)carbamate;
2-methylpropyl (3-{[1-(1-methyl-1H-pyrazol-3-yl)-4-oxo-1,4-dihydropyridazin-3-yl]methyl}phenyl)carbamate;
propyl (3-{[1-(1-methyl-1H-pyrazol-3-yl)-4-oxo-1,4-dihydropyridazin-3-yl]methyl}phenyl)carbamate;
propyl {3-[(4-oxo-1-pyridin-3-yl-1,4-dihydropyridazin-3-yl]methyl}phenyl)carbamate;
1-pyridin-3-yl-3-(3-pyrimidin-2-ylbenzyl)pyridazin-4(1H)-one;
propyl {3-[(4-oxo-1-pyridin-4-yl-1,4-dihydropyridazin-3-yl)methyl]phenyl}carbamate;
ethyl (3-{[1-(6-methoxypyridin-3-yl)-4-oxo-1,4-dihydropyridazin-3-yl]methyl}phenyl)carbamate;
propyl (3-{[1-(6-methoxypyridin-3-yl)-4-oxo-1,4-dihydropyridazin-3-yl]methyl}phenyl)carbamate;
1-(6-methoxypyridin-3-yl)-3-[3-(5-methoxypyrimidin-2-yl)benzyl]pyridazin-4(1H)-one;
ethyl(3-{[1-(5-fluoropyridin-3-yl)-4-oxo-1,4-dihydropyridazin-3-yl]methyl}phenyl)carbamate;
propyl (3-{[1-(5-fluoropyridin-3-yl)-4-oxo-1,4-dihydropyridazin-3-yl]methyl}phenyl)carbamate;
1-(5-fluoropyridin-3-yl)-3-[3-(5-methoxypyrimidin-2-yl)benzyl]pyridazin-4(1H)-one;
3-[3-(5-methoxypyrimidin-2-yl)benzyl]-1-(5-methylpyridin-3-yl)pyridazin-4(1H)-one;
1-(1-methyl-1H-pyrazol-4-yl)-3-[3-(pyrazin-2-yl)benzyl]pyridazin-4(1H)-one;
1-(1-methyl-1H-pyrazol-4-yl)-3-[3-(1-methyl-1H-pyrazol-3-yl)benzyl]pyridazin-4(1H)-one;
1-(1-methyl-1H-pyrazol-4-yl)-3-[3-(2-methylpyrimidin-4-yl)benzyl]pyridazin-4(1H)-one;
3-[3-(3-methyl-1,2,4-oxadiazol-5-yl)benzyl]-1-(1-methyl-1H-pyrazol-4-yl)pyridazin-4(1H)-one;
3-[3-(5-methyl-1,2,4-oxadiazol-3-yl)benzyl]-1-(1-methyl-1H-pyrazol-4-yl)pyridazin-4(1H)-one;
1-(4-chlorophenyl)-3-(quinolin-6-ylmethyl)pyridazin-4(1H)-one
1-(1-methyl-1H-pyrazol-4-yl)-3-[(2-methylquinolin-5-yl)methyl]pyridazin-4(1H)-one;
4-{[1-(1-methyl-1H-pyrazol-4-yl)-4-oxo-1,4-dihydropyridazin-3-yl]methyl}-2,3-dihydro-1H-isoindol-1-one;
3-(imidazo[1,2-a]pyridin-6-ylmethyl)-1-(1-methyl-1H-pyrazol-4-yl)pyridazin-4(1H)-one;
ethyl 2-fluoro-3-{[1-(1-methyl-1H-pyrazol-4-yl)-4-oxo-1,4-dihydropyridazin-3-yl]methyl}benzoate;
2-(3-{[1-(1-methyl-1H-pyrazol-4-yl)-4-oxo-1,4-dihydropyridazin-3-yl]methyl}phenyl)acetamide;
3-[(2-methyl-2H-indazol-5-yl)methyl]-1-(1-methyl-1H-pyrazol-4-yl)pyridazin-4(1H)-one;
3-(1 H-indazol-4-ylmethyl)-1-(1-methyl-1H-pyrazol-4-yl)pyridazin-4(1H)-one;
3-(1-benzofuran-5-ylmethyl)-1-(1-methyl-1H-pyrazol-4-yl)pyridazin-4(1H)-one;
propyl (3-{[1-(1-methyl-1H-pyrazol-4-yl)-4-oxo-1,4-dihydropyridazin-3-yl]methyl}phenyl)carbamate;
2-methylpropyl (3-{[1-(1-methyl-1H-pyrazol-4-yl)-4-oxo-1,4-dihydropyridazin-3-yl]methyl}phenyl)carbamate;
2-methoxyethyl(3-{[1-(1-methyl-1H-pyrazol-4-yl)-4-oxo-1,4-dihydropyridazin-3-yl]methyl}phenyl)carbamate;
1-(1-methyl-1H-pyrazol-4-yl)-3-(quinolin-6-ylmethyl)pyridazin-4(1H)-one;
propyl (3-{[1-(2,6-dichloropyridin-4-yl)-4-oxo-1,4-dihydropyridazin-3-yl]methyl}phenyl)carbamate;
1-(1-methyl-1H-pyrazol-4-yl)-3-[3-(1-propyl-1H-1,2,4-triazol-3-yl)benzyl]pyridazin-4(1H)-one;
1-(1-methyl-1H-pyrazol-4-yl)-3-[3-(2-methyl-2H-tetrazol-5-yl)benzyl]pyridazin-4(1H)-one;
3-[(3-ethoxyquinolin-6-yl)methyl]-1-(1-methyl-1H-pyrazol-4-yl)pyridazin-4(1H)-one;
1-(1-methyl-1H-pyrazol-4-yl)-3-{[3-(2-morpholin-4-ylethoxy)quinolin-6-yl]methyl}pyridazin-4(1H)-one;
3-{[3-(2-methoxyethoxy)quinolin-6-yl]methyl}-1-(1-methyl-1H-pyrazol-4-yl)pyridazin-4(1H)-one;
3-({3-[(3-methyloxetan-3-yl)methoxy]quinolin-6-yl}methyl)-1-(1-methyl-1H-pyrazol-4-yl)pyridazin-4(1H)-one;
1-(1-methyl-1H-pyrazol-4-yl)-3-[(3-propoxyquinolin-6-yl)methyl]pyridazin-4(1H)-one;
rac-1-(1-methyl-1H-pyrazol-4-yl)-3-{[3-(tetrahydrofuran-3-ylmethoxy)quinolin-6-yl]methyl}pyridazin-4(1H)-one;
3-[(3-ethoxyquinolin-6-yl)methyl]-1-(3,4,5-trifluorophenyl)pyridazin-4(1H)-one;
3-{3-[1-(2-methoxyethyl)-1H-1,2,4-triazol-3-yl]benzyl}-1-(1-methyl-1H-pyrazol-4-yl)pyridazin-4(1H)-one;
3-{3-[5-(benzyloxy)pyrimidin-2-yl]benzyl}-1-(1-methyl-1H-pyrazol-4-yl)pyridazin-4(1H)-one;
2-methylpropyl (3-{[1-(4-bromophenyl)-4-oxo-1,4-dihydropyridazin-3-yl]methyl}phenyl)carbamate;
2-methylpropyl (3-{[1-(3-bromophenyl)-4-oxo-1,4-dihydropyridazin-3-yl]methyl}phenyl)carbamate;
2-methoxyethyl(3-{[1-(1-ethyl-1H-pyrazol-4-yl)-4-oxo-1,4-dihydropyridazin-3-yl]methyl}phenyl)carbamate;
3-{3-[5-(benzyloxy)pyrimidin-2-yl]benzyl}-1-(1-ethyl-1H-pyrazol-4-yl)pyridazin-4(1H)-one;
3-[3-[3-(5-ethoxypyrimidin-2-yl)benzyl]-4-oxopyridazin-1(4H)-yl]benzonitrile;
2-methoxyethyl(3-{[1-(3-cyano-5-fluorophenyl)-4-oxo-1,4-dihydropyridazin-3-yl]methyl}phenyl)carbamate;
3-(isoquinolin-6-ylmethyl)-1-(1-methyl-1H-pyrazol-4-yl)pyridazin-4(1H)-one;
1-(1-methyl-1H-pyrazol-4-yl)-3-[3-(5-methyl-1,3,4-thiadiazol-2-yl)benzyl]pyridazin-4(1H)-one;
3-[3-(1-butyl-1H-1,2,4-triazol-3-yl)benzyl]-1-(1-methyl-1H-pyrazol-4-yl)pyridazin-4(1H)-one;
3-{3-[1-(3-methoxypropyl)-1H-1,2,4-triazol-3-yl]benzyl}-1-(1-methyl-1H-pyrazol-4-yl)pyridazin-4(1H)-one;
3-{3-[1-(3-methylbutyl)-1H-1,2,4-triazol-3-yl]benzyl}-1-(4-methyl-1H-pyrazol-4-yl)pyridazin-4(1H)-one;
rac-1-(1-methyl-1H-pyrazol-4-yl)-3-{3-[1-(tetrahydrofuran-3-ylmethyl)-TH-1,2,4-triazol-3-yl]benzyl}pyridazin-4(1H)-one;
1-(3,4-difluorophenyl)-3-[3-(1-propyl-1H-1,2,4-triazol-3-yl)benzyl]pyridazin-4(1H)-one;
3-fluoro-5-{4-oxo-3-[3-(1-propyl-1H-1,2,4-triazol-3-yl)benzyl]pyridazin-1(4H)-yl}benzonitrile;
1-(3,4-difluorophenyl)-3-[3-(1-ethyl-1H-1,2,4-triazol-3-yl)benzyl]pyridazin-4(1H)-one;
3-{3-[3-(1-ethyl-1H-1,2,4-triazol-3-yl)benzyl]-4-oxopyridazin-1(4H)-yl}-5-fluorobenzonitrile;
3-{3-[3-(1-ethyl-1H-1,2,4-triazol-3-yl)benzyl]-4-oxopyridazin-1(4H)-yl}benzonitrile;
3-{(4-oxo-3-[3-(1-propyl-1H-1,2,4-triazol-3-yl)benzyl]pyridazin-1(4H)-yl}benzonitrile;
1-(1-ethyl-1H-pyrazol-4-yl)-3-[3-(1-ethyl-1H-1,2,4-triazol-3-yl)benzyl]pyridazin-4(1H)-one;
1-(1-ethyl-1H-pyrazol-4-yl)-3-[3-(1-propyl-1H-1,2,4-triazol-3-yl)benzyl]pyridazin-4(1H)-one;
1-{1-[2-(benzyloxy)ethyl]-1H-pyrazol-4-yl}-3-[3-(1-ethyl-H-1,2,4-triazol-3-yl)benzyl]pyridazin-4(1H)-one;
1-{1-[2-(benzyloxy)ethyl]-1H-pyrazol-4-yl}-3-[3-(1-propyl-1N-1,2,4-triazol-3-yl)benzyl]pyridazin-4(1H)-one;
2-methylpropyl[3-({4-oxo-1-[1-(tetrahydro-2H-pyran-4-yl)-1H-pyrazol-4-yl]-1,4-dihydropyridazin-3-yl}methyl)phenyl]carbamate;
ethyl[3-({4-oxo-1-[1-(tetrahydro-2H-pyran-4-yl)-1H-pyrazol-4-yl]-1,4-dihydropyridazin-3-yl}methyl)phenyl]carbamate;
ethyl[3-({1-[1-(2-methylpropyl)-1H-pyrazol-4-yl]-4-oxo-1,4-dihydropyridazin-3-yl}methyl)phenyl]carbamate;
2-methylpropyl[3-({4-oxo-1-[1-(2,2,2-trifluoroethyl)-1H-pyrazol-4-yl]-1,4-dihydropyridazin-3-yl}methyl)phenyl]carbamate;
2-methylpropyl[3-({1-[1-(2-methylpropyl)-1H-pyrazol-4-yl]-4-oxo-1,4-dihydropyridazin-3-yl}methyl)phenyl]carbamate;
3-[3-(5-methoxypyrimidin-2-yl)benzyl]-1-[1-(2-methylpropyl)-1H-pyrazol-4-yl]pyridazin-4(1H)-one;
2-morpholin-4-ylethyl[3-({1-[1-(2-methylpropyl)-1H-pyrazol-4-yl]-4-oxo-1,4-dihydropyridazin-3-yl}methyl)phenyl]carbamate;
rac-ethyl[3-({4-oxo-1-[1-(tetrahydrofuran-3-yl)-1H-pyrazol-4-yl]-1,4-dihydropyridazin-3-yl}methyl)phenyl]carbamate;
rac-propyl[3-({4-oxo-1-[1-(tetrahydrofuran-3-yl)-1H-pyrazol-4-yl]-1,4-dihydropyridazin-3-yl}methyl)phenyl]carbamate;
rac-2-methylpropyl[3-({4-oxo-1-[1-(tetrahydrofuran-3-yl)-1H-pyrazol-4-yl]-1,4-dihydropyridazin-3-yl}methyl)phenyl]carbamate;
ethyl[3-({4 oxo-1-[1-(tetrahydro-2H-pyran-4-ylmethyl)-1H-pyrazol-4-yl]-1,4-dihydropyridazin-3-yl}methyl)phenyl]carbamate;
propyl[3-({4-oxo-1-[1-(tetrahydro-2H-pyran-4-ylmethyl)-1H-pyrazol-4-yl]-1,4-dihydropyridazin-3-yl}methyl)phenyl]carbamate;
2-methylpropyl[3-({4-oxo-1-[1-(tetrahydro-2H-pyran-4-ylmethyl)-1H-pyrazol-4-yl]-1,4-dihydropyridazin-3-yl}methyl)phenyl]carbamate;
ethyl[3-({1-[1-(1-methylethyl)-1H-pyrazol-4-yl]-4-oxo-1,4-dihydropyridazin-3-yl}methyl)phenyl]carbamate;
propyl[3-({1-[1-(1-methylethyl)-1H-pyrazol-4-yl]-4-oxo-1,4-dihydropyridazin-3-yl}methyl)phenyl]carbamate;
2-methylpropyl[3-({1-[1-(1-methylethyl)-1H-pyrazol-4-yl]-4-oxo-1,4-dihydropyridazin-3-yl}methyl)phenyl]carbamate;
ethyl[3-({1-[1-(2-methoxyethyl)-1H-pyrazol-4-yl]-4-oxo-1,4-dihydropyridazin-3-yl}methyl)phenyl]carbamate;
propyl[3-({1-[1-(2-methoxyethyl)-1H-pyrazol-4-yl]-4-oxo-1,4-dihydropyridazin-3-yl}methyl)phenyl]carbamate;
2-methylpropyl[3-({1-[1-(2-methoxyethyl)-1H-pyrazol-4-yl]-4-oxo-1,4-dihydropyridazin-3-yl}methyl)phenyl]carbamate;
ethyl(3-{[1-(1-ethyl-1H-pyrazol-4-yl)-4-oxo-1,4-dihydropyridazin-3-yl]methyl}phenyl)carbamate;
2-methylpropyl (3-{([1-(1-ethyl-1H-pyrazol-4-yl)-4-oxo-1,4-dihydropyridazin-3-yl]methyl}phenyl)carbamate;
ethyl[3-({4-oxo-1-[1-(2,2,2-trifluoroethyl)-1H-pyrazol-4-yl]-1,4-dihydropyridazin-3-yl}methyl)phenyl]carbamate;
ethyl(3-{[4-oxo-1-(1-propyl-1H-pyrazol-4-yl)-1,4-dihydropyridazin-3-yl]methyl}phenyl)carbamate;
2-methylpropyl (3-{[4-oxo-1-(1-propyl-1H-pyrazol-4-yl)-1,4-dihydropyridazin-3-yl]methyl}phenyl)carbamate;
3-[3-(5-methoxypyrimidin-2-yl)benzyl]-1-(1-propyl-1H-pyrazol-4-yl)pyridazin-4(1H)-one;
2-morpholin-4-ylethyl(3-{[1-(1-ethyl-1H-pyrazol-4-yl)-4-oxo-1,4-dihydropyridazin-3-yl]methyl}phenyl)carbamate;
2-morpholin-4-ylethyl[3-({4-oxo-1-[1-(2,2,2-trifluoroethyl)-1H-pyrazol-4-yl]-1,4-dihydropyridazin-3-yl}methyl)phenyl]carbamate;
2-morpholin-4-ylethyl(3-{[4-oxo-1-(1-propyl-1H-pyrazol-4-yl)-1,4-dihydropyridazin-3-yl]methyl}phenyl)carbamate;
3-[5-(5-ethoxypyrimidin-2-yl)-2-fluorobenzyl]-1-(1-methyl-1H-pyrazol-4-yl)pyridazin-4(1H)-one;
3-[3-(5-ethoxypyrimidin-2-yl)-4-fluorobenzyl]-1-(1-methyl-1H-pyrazol-4-yl)pyridazin-4(1H)-one;
3-[3-(5-ethoxypyrimidin-2-yl)-5-fluorobenzyl]-1-(1-methyl-1H-pyrazol-4-yl)pyridazin-4(1H)-one;
3-[3-(5-ethoxypyrimidin-2-yl)-2-fluorobenzyl]-1-(1-methyl-1H-pyrazol-4-yl)pyridazin-4(1H)-one;
3-(3-{5-[(trans-4-hydroxy-4-methylcyclohexyl)oxy]pyrimidin-2-yl}benzyl)-1-(1-methyl-1H-pyrazol-4-yl)pyridazin-4(1H)-one;
3-{3-[5-(1,4-dioxaspiro[4.5]dec-8-yloxy)pyrimidin-2-yl]benzyl}-1-(1-methyl-1H-pyrazol-4-yl)pyridazin-4(1H)-one;
3-{3-[5-(benzyloxy)pyrimidin-2-yl]benzyl}-1-(3,4-difluorophenyl)pyridazin-4(1H)-one;
3-{3-[5-(benzyloxy)pyrimidin-2-yl]benzyl}-1-(3,5-difluorophenyl)pyridazin-4(1H)-one;
3-chloro-5-{3-[3-(5-methoxypyrimidin-2-yl)benzyl]-4-oxopyridazin-1(4H)-yl}benzonitrile;
2-methoxyethyl(3-{[1-(3-chloro-5-cyanophenyl)-4-oxo-1,4-dihydropyridazin-3-yl]methyl}phenyl)carbamate;
3-fluoro-5-{3-[3-(5-methoxypyrimidin-2-yl)benzyl]-4-oxopyridazin-1(4H)-yl}benzonitrile;
2-methoxyethyl(3-{[1-(5-cyanopyridin-3-yl)-4-oxo-1,4-dihydropyridazin-3-yl]methyl}phenyl)carbamate;
rac-tetrahydrofuran-3-ylmethyl(3-{[1-(5-cyanopyridin-3-yl)-4-oxo-1,4-dihydropyridazin-3-yl]methyl}phenyl)carbamate;
rac-tetrahydrofuran-3-ylmethyl(3-{[1-(3-cyano-5-fluorophenyl)-4-oxo-1,4-dihydropyridazin-3-yl]methyl}phenyl)carbamate;
5-[3-{3-[5-(2-methoxyethoxy)pyrimidin-2-yl]benzyl}-4-oxopyridazin-1(4H)-yl]pyridine-3-carbonitrile;
3-{3-[3-(5-ethoxypyrimidin-2-yl)benzyl]-4-oxopyridazin-1(4H)-yl}-5-fluorobenzonitrile;
3-fluoro-5-[3-{3-[5-(2-methoxyethoxy)pyrimidin-2-yl]benzyl}-4-oxopyridazin-1(4H)-yl]benzonitrile;
ethyl(3-{[1-(3-cyanophenyl)-4-oxo-1,4-dihydropyridazin-3-yl]methyl}phenyl)carbamate;
5-{3-[3-(5-ethoxypyrimidin-2-yl)benzyl]-4-oxopyridazin-1(4H)-yl}pyridine-3-carbonitrile;
4-{3-[3-(5-ethoxypyrimidin-2-yl)benzyl]-4-oxopyridazin-1(4H)-yl}benzonitrile;
4-[3-{3-[5-(2-methoxyethoxy)pyrimidin-2-yl]benzyl}-4-oxopyridazin-1(4H)-yl]benzonitrile;
rac-tetrahydrofuran-3-ylmethyl(3-{[1-(4-cyanophenyl)-4-oxo-1,4-dihydropyridazin-3-yl]methyl}phenyl)carbamate;
3-chloro-5-{3-[3-(5-ethoxypyrimidin-2-yl)benzyl]-4-oxopyridazin-1(4H)-yl}benzonitrile;
rac-tetrahydrofuran-3-ylmethyl(3-{[1-(3-chloro-5-cyanophenyl)-4-oxo-1,4-dihydropyridazin-3-yl]methyl}phenyl)carbamate;
3-[(4-methoxyquinolin-6-yl)methyl]-1-(1-methyl-1H-pyrazol-4-yl)pyridazin-4(1H)-one;
1-(3-bromophenyl)-3-[3-(5-ethoxypyrimidin-2-yl)benzyl]pyridazin-4(1H)-one;
2-fluoro-4-[3-{-3-[5-(2-methoxyethoxy)pyrimidin-2-yl]benzyl}-4-oxopyridazin-1(4H)-yl]benzonitrile;
1-(4-bromo-3-fluorophenyl)-3-[3-(5-ethoxypyrimidin-2-yl)benzyl]pyridazin-4(1H)-one;
1-(3,5-difluorophenyl)-3-[3-(5-ethoxypyrimidin-2-yl)benzyl]pyridazin-4(1H)-one;
1-(4-bromo-3,5-difluorophenyl)-3-[3-(5-ethoxypyrimidin-2-yl)benzyl]pyridazin-4(1H)-one;
4-{3-[3-(5-ethoxypyrimidin-2-yl)benzyl]-4-oxopyridazin-1(4H)-yl}-2-fluorobenzonitrile;
1-(3,5-difluorophenyl)-3-{3-[5-(2-methoxyethoxy)pyrimidin-2-yl]benzyl}pyridazin-4(1H)-one;
3-[3-(5-ethoxypyrimidin-2-yl)benzyl]-1-(1-ethyl-1H-pyrazol-4-yl)pyridazin-4(1H)-one;
1-(1-ethyl-1H-pyrazol-4-yl)-3-{3-[5-(2-methoxyethoxy)pyrimidin-2-yl]benzyl}pyridazin-4(1H)-one;
1-(4-chloro-3-fluorophenyl)-3-[3-(5-ethoxypyrimidin-2-yl)benzyl]pyridazin-4(1H)-one;
1-(4-chloro-3-fluorophenyl)-3-{3-[5-(2-methoxyethoxy)pyrimidin-2-yl]benzyl}pyridazin-4(1H)-one;
1-(1-methyl-1H-pyrazol-4-yl)-3-{3-[5-(1-methyl-1H-pyrazol-4-yl)pyrimidin-2-yl]benzyl}pyridazin-4(1H)-one;
3-fluoro-5-[3-({3-[5-(1-methyl-1H-pyrazol-4-yl)pyrimidin-2-yl]benzyl}-4-oxopyridazin-1(4H)-yl]benzonitrile;
1-(3,4-difluorophenyl)-3-{3-[5-(1-methyl-1H-pyrazol-4-yl)pyrimidin-2-yl]benzyl}pyridazin-4(1H)-one;
1-(1-ethyl-1H-pyrazol-4-yl)-3-{3-[5-(1-methyl-1H-pyrazol-4-yl)pyrimidin-2-yl]benzyl}pyridazin-4(1H)-one;
3-chloro-5-[3-({3-[5-(1-methyl-1H-pyrazol-4-yl)pyrimidin-2-yl]benzyl}-4-oxopyridazin-1(4H)-yl]benzonitrile;
tert-butyl[2-(3-{[1-(1-methyl-1H-pyrazol-4-yl)-4-oxo-1,4-dihydropyridazin-3-yl]methyl}phenyl)pyrimidin-5-yl]carbamate;
3-[(3-ethoxyquinolin-6-yl)methyl]-1-(1-ethyl-1H-pyrazol-4-yl)pyridazin-4(1H)-one;
3-{3-[5-(methoxymethyl)pyrimidin-2-yl]benzyl}-1-(1-methyl-1H-pyrazol-4-yl)pyridazin-4(1H)-one;
3-{3-[5-(ethoxymethyl)pyrimidin-2-yl]benzyl}-1-(1-methyl-1H-pyrazol-4-yl)pyridazin-4(1H)-one;
1-(1-ethyl-1H-pyrazol-4-yl)-3-{3-[5-(methoxymethyl)pyrimidin-2-yl]benzyl}pyridazin-4(1H)-one;
1-(1-methyl-1H-pyrazol-4-yl)-3-{3-[5-(oxetan-3-yloxy)pyrimidin-2-yl]benzyl}pyridazin-4(1H)-one;
1-(1-ethyl-1H-pyrazol-4-yl)-3-{3-[5-(oxetan-3-yloxy)pyrimidin-2-yl]benzyl}pyridazin-4(1H)-one;
rac-3-{3-[5-(1-methoxyethyl)pyrimidin-2-yl]benzyl}-1-(1-methyl-1H-pyrazol-4-yl)pyridazin-4(1H)-one;
3-{3-[5-(1-hydroxy-1-methylethyl)pyrimidin-2-yl]benzyl}-1-(1-methyl-1H-pyrazol-4-yl)pyridazin-4(1H)-one;
3-{3-[5-(1-methoxy-1-methylethyl)pyrimidin-2-yl]benzyl}-1-(1-methyl-1H-pyrazol-4-yl)pyridazin-4(1H)-one;
3-{3-[5-(1-ethoxy-1-methylethyl)pyrimidin-2-yl]benzyl}-1-(1-methyl-1H-pyrazol-4-yl)pyridazin-4(1H)-one;
3-fluoro-5-[3-(3-{5-[(1-methylpiperidin-4-yl)methoxy]pyrimidin-2-yl}benzyl)-4-oxopyridazin-1(4H)-yl]benzonitrile;
3-[3-(3-{5-[(1-methylpiperidin-4-yl)methoxy]pyrimidin-2-yl}benzyl)-4-oxopyridazin-1(4H)-yl]benzonitrile;
1-(1-ethyl-1H-pyrazol-4-yl)-3-(3-{5-[(1-methylpiperidin-4-yl)methoxy]pyrimidin-2-yl}benzyl)pyridazin-4(1H)-one;
1-(3,5-difluorophenyl)-3-(3-{5-[(1-methylpiperidin-4-yl)methoxy]pyrimidin-2-yl}benzyl)pyridazin-4(1H)-one;
rac-3-{3-[5-(2,5-dihydrofuran-2-yl)pyrimidin-2-yl]benzyl}-1-(1-methyl-1H-pyrazol-4-yl)pyridazin-4(1H)-one;
3-{3-[5-(2,5-dihydrofuran-3-yl)pyrimidin-2-yl]benzyl}-1-(1-methyl-1H-pyrazol-4-yl)pyridazin-4(1H)-one;
3-[3-(4-butyl-5-ethoxypyrimidin-2-yl)benzyl]-1-(1-methyl-1H-pyrazol-4-yl)pyridazin-4(1H)-one;
3-[3-(5-ethoxy-4-methylpyrimidin-2-yl)benzyl]-1-(1-methyl-1H-pyrazol-4-yl)pyridazin-4(1H)-one;
3-[3-(5-ethoxy-4-ethylpyrimidin-2-yl)benzyl]-1-(1-methyl-1H-pyrazol-4-yl)pyridazin-4(1H)-one;
methyl 4-{4-[3-(3-{[(2-methylpropoxy)carbonyl]amino}benzyl)-4-oxopyridazin-1(4H)-yl]-1H-pyrazol-1-yl}butanoate;
4-{4-[3-(3-{[(2-methylpropoxy)carbonyl]amino}benzyl)-4-oxopyridazin-1(4H)-yl]-1H-pyrazol-1-yl}butanoic acid;
{4-[3-(3-{[(2-methylpropoxy)carbonyl]amino}benzyl)-4-oxopyridazin-1(4H)-yl]-1H-pyrazol-1-yl}acetic acid;
3-{4-[3-(3-{[(2-methylpropoxy)carbonyl]amino}benzyl)-4-oxopyridazin-1(4H)-yl]-1H-pyrazol-1-yl}propanoic acid;
3-[3-(5-aminopyrimidin-2-yl)benzyl]-1-(1-methyl-1H-pyrazol-4-yl)pyridazin-4(1H)-one;
3-(1H-indazol-5-ylmethyl)-1-(1-methyl-1H-pyrazol-4-yl)pyridazin-4(1H)-one;
1-(1-methyl-1H-pyrazol-4-yl)-3-[3-(5-propyl-1H-1,2,4-triazol-3-yl)benzyl]pyridazin-4(1H)-one;
1-(1-methyl-1H-pyrazol-4-yl)-3-[3-(1H-1,2,4-triazol-3-yl)benzyl]pyridazin-4(1H)-one;
3-[3-(5-hydroxypyrimidin-2-yl)benzyl]-1-(1-methyl-1H-pyrazol-4-yl)pyridazin-4(1H)-one;
1-(3,4-difluorophenyl)-3-[3-(5-hydroxypyrimidin-2-yl)benzyl]pyridazin-4(1H)-one;
1-(3,5-difluorophenyl)-3-[3-(5-hydroxypyrimidin-2-yl)benzyl]pyridazin-4(1H)-one;
3-{3-[5-(hydroxymethyl)pyrimidin-2-yl]benzyl}-1-(1-methyl-1H-pyrazol-4-yl)pyridazin-4(1H)-one;
3-[3-(1-ethyl-1H-1,2,4-triazol-3-yl)benzyl]-1-[1-(2-hydroxyethyl)-1H-pyrazol-4-yl]pyridazin-4(1H)-one;
1-[1-(2-hydroxyethyl)-1H-pyrazol-4-yl]-3-[3-(1-propyl-1H-1,2,4-triazol-3-yl)benzyl]pyridazin-4(1H)-one;
3-[3-(5-ethoxypyrimidin-2-yl)benzyl]-1-[1-(2-hydroxyethyl)-1H-pyrazol-4-yl]pyridazin-4(1H)-one;
3-[3-(5-hydroxypyrimidin-2-yl)benzyl]-1-(1H-pyrazol-4-yl)pyridazin-4(1H)-one;
3-[3-(5-ethoxypyrimidin-2-yl)benzyl]-1-(1H-pyrazol-4-yl)pyridazin-4(1H)-one;
2-methoxyethyl(3-{[4-oxo-1-(1H-pyrazol-4-yl)-1,4-dihydropyridazin-3-yl]methyl}phenyl)carbamate;
isobutyl (3-{[4-oxo-1-(1H-pyrazol-4-yl)-1,4-dihydropyridazin-3-yl]methyl}phenyl)carbamate;
rac-1-(1-methyl-1H-pyrazol-4-yl)-3-{(3-[5-(tetrahydrofuran-2-yl)pyrimidin-2-yl]benzyl}pyridazin-4(1H)-one;
rac-1-(1-methyl-1H-pyrazol-4-yl)-3-{3-[5-(tetrahydrofuran-3-yl)pyrimidin-2-yl]benzyl}pyridazin-4(1H)-one;
1-(1-methyl-1H-pyrazol-4-yl)-3-{3-[5-(piperidin-4-yl)pyrimidin-2-yl]benzyl}pyridazin-4(1H)-one;
3-{(1S or R)-1-[3-(5-ethoxypyrimidin-2-yl)phenyl]ethyl}-1-(1-methyl-1H-pyrazol-4-yl)pyridazin-4(1H)-one;
3-{(1R or S)-1-[3-(5-ethoxypyrimidin-2-yl)phenyl]ethyl}-1-(1-methyl-1H-pyrazol-4-yl)pyridazin-4(1H)-one;
3-{(1S or R)-1-[3-(5-methoxypyrimidin-2-yl)phenyl]ethyl}-1-(1-methyl-1H-pyrazol-4-yl)pyridazin-4(1H)-one;
3-{(1R or S)-1-[3-(5-methoxypyrimidin-2-yl)phenyl]ethyl}-1-(1-methyl-1H-pyrazol-4-yl)pyridazin-4(1H)-one;
3-{(1S or R)-1-[3-(5-ethoxypyrimidin-2-yl)phenyl]propyl}-1-(1-methyl-1H-pyrazol-4-yl)pyridazin-4(1H)-one;
3-{(1R or S)-1-[3-(5-ethoxypyrimidin-2-yl)phenyl]propyl}-1-(1-methyl-1H-pyrazol-4-yl)pyridazin-4(1H)-one;
3-{(1S or R)-1-[3-(5-ethoxypyrimidin-2-yl)phenyl]butyl}-1-(1-methyl-1H-pyrazol-4-yl)pyridazin-4(1H)-one;
3-{(1R or S)-1-[3-(5-ethoxypyrimidin-2-yl)phenyl]butyl}-t-(1-methyl-1H-pyrazol-4-yl)pyridazin-4(1H)-one;
3-[(1S or R)-1-{3-[5-(difluoromethoxy)pyrimidin-2-yl]phenyl}ethyl]-1-(1-methyl-1H-pyrazol-4-yl)pyridazin-4(1H)-one;
3-[(1R or S)-1-{3-[5-(difluoromethoxy)pyrimidin-2-yl]phenyl}ethyl]-1-(1-methyl-1H-pyrazol-4-yl)pyridazin-4(1H)-one;
1-(1-methyl-1H-pyrazol-4-yl)-3-[(1S or R)-1-{3-[5-(tetrahydro-2H-pyran-4-yloxy)pyrimidin-2-yl]phenyl}ethyl]pyridazin-4(1H)-one;
1-(1-methyl-1H-pyrazol-4-yl)-3-[(1R or S)-1-{3-[5-(tetrahydro-2H-pyran-4-yloxy)pyrimidin-2-yl]phenyl}ethyl]pyridazin-4(1H)-one;
1-(1-ethyl-1H-pyrazol-4-yl)-3-((1S or R)-1-{3-[5-(oxetan-3-yloxy)pyrimidin-2-yl]phenyl}ethyl)pyridazin-4(1H)-one;
1-(1-ethyl-1H-pyrazol-4-yl)-3-((1R or S)-1-{3-[5-(oxetan-3-yloxy)pyrimidin-2-yl]phenyl}ethyl)pyridazin-4(1H)-one;
1-(1-methyl-1H-pyrazol-4-yl)-3-((1S or R)-1-{3-[5-(oxetan-3-yloxy)pyrimidin-2-yl]phenyl}ethyl)pyridazin-4(1H)-one;
1-(1-methyl-1H-pyrazol-4-yl)-3-((1R or S)-1-{3-[5-(oxetan-3-yloxy)pyrimidin-2-yl]phenyl}ethyl)pyridazin-4(1H)-one;
1-(1-ethyl-1H-pyrazol-4-yl)-3-((1S or R)-1-{3-[5-(methoxymethyl)pyrimidin-2-yl]phenyl}ethyl)pyridazin-4(1H)-one;
1-(1-ethyl-1H-pyrazol-4-yl)-3-((1R or S)-1-{3-[5-(methoxymethyl)pyrimidin-2-yl]phenyl}ethyl)pyridazin-4(1H)-one;
3-((1S or R)-1-{3-[5-(methoxymethyl)pyrimidin-2-yl]phenyl}ethyl)-1-(1-methyl-1H-pyrazol-4-yl)pyridazin-4(1H)-one;
3-((1R or S)-1-{3-[5-(methoxymethyl)pyrimidin-2-yl]phenyl}ethyl)-1-(1-methyl-1H-pyrazol-4-yl)pyridazin-4(1H)-one;
rac-3-(1-{3-[5-(ethoxymethyl)pyrimidin-2-yl]phenyl}ethyl)-1-(1-methyl-1H-pyrazol-4-yl)pyridazin-4(1H)-one;
3-((1S or R)-1-{3-[5-(2-methoxyethoxy)pyrimidin-2-yl]phenyl}ethyl)-1-(1-methyl-1H-pyrazol-4-yl)pyridazin-4(1H)-one;
3-((1R or S)-1-{3-[5-(2-methoxyethoxy)pyrimidin-2-yl]phenyl}ethyl)-(1-methyl-1H-pyrazol-4-yl)pyridazin-4(1H)-one;
rac-3-[3-{1-[3-(5-ethoxypyrimidin-2-yl)phenyl]ethyl}-4-oxopyridazin-1(4H)-yl]benzonitrile;
rac-3-{1-[3-(5-methoxypyrimidin-2-yl)phenyl]propyl}-1-(1-methyl-1H-pyrazol-4-yl)pyridazin-4(1H)-one;
3-[(1S or R)-1-(3-ethoxyquinolin-6-yl)ethyl]-1-(1-ethyl-1H-pyrazol-4-yl)pyridazin-4(1H)-one;
3-[(1R or S)-1-(3-ethoxyquinolin-6-yl)ethyl]-1-(1-ethyl-1H-pyrazol-4-yl)pyridazin-4(1H)-one;
3-((1S or R)-1-{3-[5-(benzyloxy)pyrimidin-2-yl]phenyl}ethyl)-1-(1-ethyl-1H-pyrazol-4-yl)pyridazin-4(1H)-one;
3-((1R or S)-1-{3-[5-(benzyloxy)pyrimidin-2-yl]phenyl}ethyl)-1-(1-ethyl-1H-pyrazol-4-yl)pyridazin-4(1H)-one;
1-(3,4-difluorophenyl)-3-{((1S or R)-1-[3-(5-ethylpyrimidin-2-yl)phenyl]ethyl}pyridazin-4(1H)-one;
1-(3,4-difluorophenyl)-3-{(1R or S)-1-[3-(5-ethylpyrimidin-2-yl)phenyl]ethyl}pyridazin-4(1H)-one;
1-(1-methyl-1H-pyrazol-4-yl)-3-[(1S or R)-1-{3-[5-(1-methyl-1H-pyrazol-4-yl)pyrimidin-2-yl]phenyl}ethyl]pyridazin-4(1H)-one;
1-(1-methyl-1H-pyrazol-4-yl)-3-[(1R or S)-1-{3-[5-(1-methyl-1H-pyrazol-4-yl)pyrimidin-2-yl]phenyl}ethyl]pyridazin-4(1H)-one;
1-(1-ethyl-1H-pyrazol-4-yl)-3-[(1S or R)-1-{3-[5-(1-methyl-1H-pyrazol-4-yl)pyrimidin-2-yl]phenyl}ethyl]pyridazin-4(1 h)-one;
1-(1-ethyl-1H-pyrazol-4-yl)-3-[(1R or S)-1-{3-[5-(1-methyl-1H-pyrazol-4-yl)pyrimidin-2-yl]phenyl}ethyl]pyridazin-4(1H)-one;
3-{-(1S or R)-1-[3-(5-ethylpyrimidin-2-yl)phenyl]ethyl}-1-(1-methyl-1H-pyrazol-4-yl)pyridazin-4(1H)-one;
3-{(1R or S)-1-[3-(5-ethylpyrimidin-2-yl)phenyl]ethyl}-1-(1-methyl-1H-pyrazol-4-yl)pyridazin-4(1-1)-one;
1-(3,4-difluorophenyl)-3-[(1S or R)-1-{3-[5-(1-methyl-1H-pyrazol-4-yl)pyrimidin-2-yl]phenyl}ethyl]pyridazin-4(1H)-one;
1-(3,4-difluorophenyl)-3-[(1R or S)-1-{3-[5-(1-methyl-1H-pyrazol-4-yl)pyrimidin-2-yl]phenyl}ethyl]pyridazin-4(1H)-one;
1-(1-methyl-1H-pyrazol-4-yl)-3-[(1-R or S)-1-{3-[5-(4H-1,2,4-triazol-4-yl)pyrimidin-2-yl]phenyl}ethyl]pyridazin-4(1H)-one;
3-{(1S or R)-1-[3-(5-bromopyrimidin-2-yl)phenyl]ethyl}-1-(1-methyl-1H-pyrazol-4-yl)pyridazin-4(1H)-one;
3-{(1R or S)-1-[3-(5-bromopyrimidin-2-yl)phenyl]ethyl}-1-(1-methyl-1H-pyrazol-4-yl)pyridazin-4(1H)-one;
1-(1-methyl-1H-pyrazol-4-yl)-3-{(1S or R)-1-[3-(5-morpholin-4-ylpyrimidin-2-yl)phenyl]ethyl}pyridazin-4(1H)-one;
1-(1-methyl-1H-pyrazol-4-yl)-3-{(1R or S)-1-[3-(5-morpholin-4-ylpyrimidin-2-yl)phenyl]ethyl}pyridazin-4(1H)-one;
3-{(1S or R)-1-[3-(5-ethoxypyrimidin-2-yl)phenyl]ethyl}-1-(1-ethyl-1H-pyrazol-4-yl)pyridazin-4(1H)-one;
3-{((1R or S)-1-[3-(5-ethoxypyrimidin-2-yl)phenyl]ethyl}-1-(1-ethyl-1H-pyrazol-4-yl)pyridazin-4(1H)-one;
1-(1-ethyl-1H-pyrazol-4-yl)-3-((1S or R)-1-{(3-[5-(2-methoxyethoxy)pyrimidin-2-yl]phenyl}ethyl)pyridazin-4(1H)-one;
1-(1-ethyl-1H-pyrazol-4-yl)-3-((1R or S)-1-{3-[5-(2-methoxyethoxy)pyrimidin-2-yl]phenyl}ethyl)pyridazin-4(1H)-one;
1-(3,4-difluorophenyl)-3-[(1S or R)-1-{3-[5-(2-hydroxy-2-methylpropoxy)pyrimidin-2-yl]phenyl}ethyl]pyridazin-4(1H)-one;
1-(3,4-difluorophenyl)-3-[(1R or S)-1-{3-[5-(2-hydroxy-2-methylpropoxy)pyrimidin-2-yl]phenyl}ethyl]pyridazin-4(1H)-one;
1-(3,4-difluorophenyl)-3-[(1S or R)-1-{3-[5-(2-methoxy-2-methylpropoxy)pyrimidin-2-yl]phenyl}ethyl]pyridazin-4(1H)-one;
1-(3,4-difluorophenyl)-3-[(R or S)-1-{3-[5-(2-methoxy-2-methylpropoxy)pyrimidin-2-yl]phenyl}ethyl]pyridazin-4(1H)-one;
rac-3-(3-hydroxy-1-{3-[5-(2-methoxyethoxy)pyrimidin-2-yl]phenyl}propyl)-1-(1-methyl-1H-pyrazol-4-yl)pyridazin-4(1H)-one;
rac-3-{2-hydroxy-1-[3-(5-methoxypyrimidin-2-yl)phenyl]ethyl}-1-(1-methyl-1H-pyrazol-4-yl)pyridazin-4(1H)-one;
3-{(1S or R)-1-[3-(5-ethoxypyrimidin-2-yl)phenyl]ethyl}-1-[1-(2-hydroxyethyl)-1H-pyrazol-4-yl]pyridazin-4(1H)-one;
3-{(1R or S)-1-[3-(5-ethoxypyrimidin-2-yl)phenyl]ethyl}-1-[1-(2-hydroxyethyl)-1H-pyrazol-4-yl]pyridazin-4(1H)-one;
1-(1-ethyl-1H-pyrazol-4-yl)-3-{((1S or R)-1-[3-(5-hydroxypyrimidin-2-yl)phenyl]ethyl}pyridazin-4(1H)-one;
1-(1-ethyl-1H-pyrazol-4-yl)-3-{(1R or S)-1-[3-(5-hydroxypyrimidin-2-yl)phenyl]ethyl}pyridazin-4(1H)-one;
3-(3-{5-[2,2-difluoro-3-(morpholin-4-yl)propoxy]pyrimidin-2-yl}benzyl)-1-(1-methyl-1H-pyrazol-4-yl)pyridazin-4(1H)-one;
rac-1-(1-methyl-1H-pyrazol-4-yl)-3-{3-[5-(tetrahydrofuran-2-ylmethoxy)pyrimidin-2-yl]benzyl}pyridazin-4(1H)-one;
3-{3-[5-(2-methylpropoxy)pyrimidin-2-yl]benzyl}-1-(1-methyl-1H-pyrazol-4-yl)pyridazin-4(1H)-one;
1-(1-methyl-1H-pyrazol-4-yl)-3-{3-[5-(tetrahydro-2H-pyran-4-ylmethoxy)pyrimidin-2-yl]benzyl}pyridazin-4(1H)-one;
1-(1-methyl-1H-pyrazol-4-yl)-3-{3-[5-(2-morpholin-4-ylethoxy)pyrimidin-2-yl]benzyl}pyridazin-4(1H)-one;
3-{3-[5-(2-hydroxyethoxy)pyrimidin-2-yl]benzyl}-1-(1-methyl-1H-pyrazol-4-yl)pyridazin-4(1H)-one;
1-(1-methyl-1H-pyrazol-4-yl)-3-(3-{5-[2-(1H-pyrazol-1-yl)ethoxy]pyrimidin-2-yl}benzyl)pyridazin-4(1H)-one;
rac-1-(1-methyl-1H-pyrazol-4-yl)-3-{3-[5-(tetrahydrofuran-3-ylmethoxy)pyrimidin-2-yl]benzyl}pyridazin-4(1H)-one;
3-{3-[5-(2-methoxy-2-methylpropoxy)pyrimidin-2-yl]benzyl}-1-(1-methyl-1H-pyrazol-4-yl)pyridazin-4(1H)-one;
3-[3-(5-{[3-(hydroxymethyl)oxetan-3-yl]methoxy}pyrimidin-2-yl)benzyl]-1-(1-methyl-1H-pyrazol-4-yl)pyridazin-4(1-H)-one;
rac-3-(3-{5-[(2,2-dimethyltetrahydro-2H-pyran-4-yl)methoxy]pyrimidin-2-yl}benzyl)-1-(1-methyl-1H-pyrazol-4-yl)pyridazin-4(1H)-one;
3-{3-[5-(1-methylethoxy)pyrimidin-2-yl]benzyl}-1-(1-methyl-1H-pyrazol-4-yl)pyridazin-4(1H)-one;
1-(1-methyl-1H-pyrazol-4-yl)-3-(3-{5-[2-(1H-1,2,4-triazol-1-yl)ethoxy]pyrimidin-2-yl}benzyl)pyridazin-4(1H)-one;
3-(3-{5-[(3-fluorooxetan-3-yl)methoxy]pyrimidin-2-yl}benzyl)-1-(1-methyl-1H-pyrazol-4-yl)pyridazin-4(1H)-one;
3-{3-[5-(2-isoxazol-4-ylethoxy)pyrimidin-2-yl]benzyl}-1-(1-methyl-1H-pyrazol-4-yl)pyridazin-4(1H)-one;
3-{3-[5-(2,2-difluoroethoxy)pyrimidin-2-yl]benzyl}-1-(1-methyl-1H-pyrazol-4-yl)pyridazin-4(1H)-one;
3-[3-(5-ethoxypyrimidin-2-yl)benzyl]-1-(1-methyl-1H-pyrazol-4-yl)pyridazin-4(1H)-one-d5;
rac-1-(1-methyl-1H-pyrazol-4-yl)-3-{3-[5-(tetrahydrofuran-3-yloxy)pyrimidin-2-yl]benzyl}pyridazin-4(1H)-one;
1-(1-methyl-1H-pyrazol-4-yl)-3-{3-[5-(tetrahydro-2H-pyran-4-yloxy)pyrimidin-2-yl]benzyl}pyridazin-4(1H)-one;
3-{3-[5-(cyclopropylmethoxy)pyrimidin-2-yl]benzyl}-1-(1-methyl-1H-pyrazol-4-yl)pyridazin-4(1H)-one;
N,N-dimethyl-2-{[2-(3-{[1-(1-methyl-1H-pyrazol-4-yl)-4-oxo-1,4-dihydropyridazin-3-yl]methyl}phenyl)pyrimidin-5-yl]oxy}acetamide;
1-(1-methyl-1H-pyrazol-4-yl)-3-{3-[5-(2-morpholin-4-yl-2-oxoethoxy)pyrimidin-2-yl]benzyl}pyridazin-4(1H)-one;
3-(3-{5-[(5-methyl-1,2,4-oxadiazol-3-yl)methoxy]pyrimidin-2-yl}benzyl)-1-(1-methyl-1H-pyrazol-4-yl)pyridazin-4(1H)-one;
3-(3-{5-[(5-cyclopropyl-1,2,4-oxadiazol-3-yl)methoxy]pyrimidin-2-yl}benzyl)-1-(1-methyl-1H-pyrazol-4-yl)pyridazin-4(1H)-one;
3-[3-(5-{[5-(1-methylethyl)-1,2,4-oxadiazol-3-yl]methoxy}pyrimidin-2-yl)benzyl]-1-(1-methyl-1H-pyrazol-4-yl)pyridazin-4(1H)-one;
3-{3-[5-(isothiazol-3-ylmethoxy)pyrimidin-2-yl]benzyl}-1-(1-methyl-1H-pyrazol-4-yl)pyridazin-4(1H)-one;
3-(3-{5-[(5-methylisoxazol-3-yl)methoxy]pyrimidin-2-yl}benzyl)-1-(1-methyl-1H-pyrazol-4-yl)pyridazin-4(1H)-one;
3-(3-{5-[(3-methylisoxazol-5-yl)methoxy]pyrimidin-2-yl}benzyl)-1-(1-methyl-1H-pyrazol-4-yl)pyridazin-4(1H)-one;
tert-butyl[2-(3-{[1-(1-methyl-1H-pyrazol-4-yl)-4-oxo-1,4-dihydropyridazin-3-yl]methyl}phenyl)pyrimidin-5-yl]oxyacetate;
tert-butyl 4-({[2-(3-{[1-(1 ethyl-1H-pyrazol-4-yl)-4-oxo-1,4-dihydropyridazin-3-yl]methyl}phenyl)pyrimidin-5-yl]oxy}methyl)piperidine-1-carboxylate;
tert-butyl 3-{[2-(3-{[1-(1-ethyl-1H-pyrazol-4-yl)-4-oxo-1,4-dihydropyridazin-3-yl]methyl}phenyl)pyrimidin-5-yl]oxy}azetidine-1-carboxylate;
tert-butyl 4-({[2-(3-{(1R or S)-1-[1-(1-ethyl-1H-pyrazol-4-yl)-4-oxo-1,4-dihydropyridazin-3-yl]ethyl}phenyl)pyrimidin-5-yl]oxy}methyl)-4-fluoropiperidine-1-carboxylate;
tert-butyl 4-({[2-(3-{(1R or S)-1-[1-(1-ethyl-1H-pyrazol-4-yl)-4-oxo-1,4-dihydropyridazin-3-yl]ethyl}phenyl)pyrimidin-5-yl]oxy}methyl)piperidine-1-carboxylate;
tert-butyl 3-{[2-(3-{(1R or S)-1-[1-(1-ethyl-1H-pyrazol-4-yl)-4-oxo-1,4-dihydropyridazin-3-yl]ethyl}phenyl)pyrimidin-5-yl]oxy}azetidine-1-carboxylate;
1-(1-ethyl-1H-pyrazol-4-yl)-3-[(1R or S)-1-{3-[5-(1-methylethoxy)pyrimidin-2-yl]phenyl}ethyl]pyridazin-4(1H)-one;
3-[(1R or S)-1-{3-[5-(2,2-difluoroethoxy)pyrimidin-2-yl]phenyl}ethyl]-1-(1-ethyl-1H-pyrazol-4-yl)pyridazin-4(1H)-one;
1-(1-ethyl-1H-pyrazol-4-yl)-3-[(1or S)-1-{3-[5-(2-hydroxyethoxy)pyrimidin-2-yl]phenyl}ethyl]pyridazin-4(1H)-one;
1-(1-ethyl-1H-pyrazol-4-yl)-3-(1-{3-[5-(oxetan-2-ylmethoxy)pyrimidin-2-yl]phenyl}ethyl)pyridazin-4(1H)-one;
1-(f-ethyl-1H-pyrazol-4-yl)-3-(1-{3-[5-(tetrahydrofuran-3-yloxy)pyrimidin-2-yl]phenyl}ethyl)pyridazin-4(1-1)-one;
1-(1-ethyl-1H-pyrazol-4-yl)-3-(1-{3-[5-(tetrahydrofuran-2-ylmethoxy)pyrimidin-2-yl]phenyl}ethyl)pyridazin-4(1H)-one;
3-(1-{3-[5-(1,4-dioxan-2-ylmethoxy)pyrimidin-2-yl]phenyl}ethyl)-1-(1-ethyl-1H-pyrazol-4-yl)pyridazin-4(1H)-one;
1-(1-ethyl-1H-pyrazol-4-yl)-3-[(1R or S)—{3-[5-(2-morpholin-4-ylethoxy)pyrimidin-2-yl]phenyl}ethyl]pyridazin-4(1H)-one;
1-(1-ethyl-1H-pyrazol-4-yl)-3-[(1R or S)-1-(3-{5-[(3-methylisoxazol-5-yl)methoxy]pyrimidin-2-yl}phenyl)ethyl]pyridazin-4(1H)-one;
1-(1-ethyl-1H-pyrazol-4-yl)-3-[(1R or S)-1-(3-{5-[(5-methyl-1,2,4-oxadiazol-3-yl)methoxy]pyrimidin-2-yl}phenyl)ethyl]pyridazin-4(1H)-one;
1-(1-ethyl-1H-pyrazol-4-yl)-3-[(1R or S)-1-(3-{5-[2-(1H-pyrazol-1-yl)ethoxy]pyrimidin-2-yl}phenyl)ethyl]pyridazin-4(1H)-one;
1-(1-ethyl-1H-pyrazol-4-yl)-3-[(1R or S)—(3-{5-[(3-methyloxetan-3-yl)methoxy]pyrimidin-2-yl}phenyl)ethyl]pyridazin-4(1H)-one;
1-(1-ethyl-1H-pyrazol-4-yl)-3-[3-(5-isopropoxypyrimidin-2-yl)benzyl]pyridazin-4(1H)-one;
3-{3-[5-(2,2-difluoroethoxy)pyrimidin-2-yl]benzyl}-1-(1-ethyl-1H-pyrazol-4-yl)pyridazin-4(1H)-one;
1-(1-ethyl-1H-pyrazol-4-yl)-3-{3-[5-(2-hydroxyethoxy)pyrimidin-2-yl]benzyl}pyridazin-4(1H)-one;
rac-1-(1-ethyl-1H-pyrazol-4-yl)-3-{3-[5-(oxetan-2-ylmethoxy)pyrimidin-2-yl]benzyl}pyridazin-4(1H)-one;
rac-1-(1-ethyl-1H-pyrazol-4-yl)-3-{3-[5-(tetrahydrofuran-3-yloxy)pyrimidin-2-yl]benzyl}pyridazin-4(1H)-one;
rac-1-(1-ethyl-1H-pyrazol-4-yl)-3-{3-[5-(tetrahydrofuran-2-ylmethoxy)pyrimidin-2-yl]benzyl}pyridazin-4(1H)-one;
rac-3-{3-[5-(1,4-dioxan-2-ylmethoxy)pyrimidin-2-yl]benzyl}-1-(1-ethyl-1H-pyrazol-4-yl)pyridazin-4(1H)-one;
1-(1-ethyl-1H-pyrazol-4-yl)-3-{3-[5-(2-morpholin-4-ylethoxy)pyrimidin-2-yl]benzyl}pyridazin-4(1H)-one;
1-(1-ethyl-1H-pyrazol-4-yl)-3-(3-{5-[(3-methylisoxazol-5-yl)methoxy]pyrimidin-2-yl}benzyl)pyridazin-4(1H)-one;
1-(1-ethyl-1H-pyrazol-4-yl)-3-(3-{5-[(5-methyl-1,2,4-oxadiazol-3-yl)methoxy]pyrimidin-2-yl}benzyl)pyridazin-4(1H)-one;
1-(1-ethyl-1H-pyrazol-4-yl)-3-(3-{5-[2-(1H-pyrazol-1-yl)ethoxy]pyrimidin-2-yl}benzyl)pyridazin-4(1H)-one;
1-(1-ethyl-1H-pyrazol-4-yl)-3-(3-{5-[(3-methyloxetan-3-yl)methoxy]pyrimidin-2-yl}benzyl)pyridazin-4(1H)-one;
1-(3,4-difluorophenyl)-3-{(3-[5-(2-methoxyethoxy)pyrimidin-2-yl]benzyl}pyridazin-4(1H)-one;
3-{3-[5-(2-methoxyethoxy)pyrimidin-2-yl-]benzyl}-1-(3,4,5-trifluorophenyl)pyridazin-4(1H)-one;
3-[3-{3-[5-(2-methoxyethoxy)pyrimidin-2-yl]benzyl}-4-oxopyridazin-1(4H)-yl]benzonitrile;
1-(3,4-difluorophenyl)-3-({3-[5-(3-methoxypropoxy)pyrimidin-2-yl]benzyl}pyridazin-4(1H)-one;
1-(3,4-difluorophenyl)-3-{3-[5-(2-ethoxyethoxy)pyrimidin-2-yl]benzyl}pyridazin-4(1H)-one;
3-[3-{3-[5-(3-methoxypropoxy)pyrimidin-2-yl]benzyl}-4-oxopyridazin-1(4H)-yl]benzonitrile;
3-[3-{3-[5-(2-ethoxyethoxy)pyrimidin-2-yl]benzyl}-4-oxopyridazin-1(4H)-yl]benzonitrile;
3-{3-[5-(3-methoxypropoxy)pyrimidin-2-yl]benzyl}-1-(3,4,5-trifluorophenyl)pyridazin-4(1H)-one;
3-{3-[5-(2-ethoxyethoxy)pyrimidin-2-yl]benzyl}-1-(3,4,5-trifluorophenyl)pyridazin-4(1H)-one;
1-(3,4-difluorophenyl)-3-{3-[5-(oxetan-3-yloxy)pyrimidin-2-yl]benzyl}pyridazin-4(1H)-one;
rac-1-(3,4-difluorophenyl)-3-{3-[5-(tetrahydrofuran-3-yloxy)pyrimidin-2-yl]benzyl}pyridazin-4(1H)-one;
rac-1-(3,4-difluorophenyl)-3 (3-{3-[5-(tetrahydrofuran-3-ylmethoxy)pyrimidin-2-yl]benzyl}pyridazin-4(1H)-one;
1-(3,4-difluorophenyl)-3-(3-({5-[(3-methyloxetan-3-yl)methoxy]pyrimidin-2-yl}benzyl)pyridazin-4(1H)-one;
1-(3,5-difluorophenyl)-3-{3-[5-(oxetan-3-yloxy)pyrimidin-2-yl]benzyl}pyridazin-4(1H)-one;
1-(3,5-difluorophenyl)-3-{3-[5-(tetrahydro-2H-pyran-4-ylmethoxy)pyrimidin-2-yl]benzyl}pyridazin-4(1H)-one;
1-(1-ethyl-1H-pyrazol-4-yl)-3-{(1S or R)-1-[3-(5-methoxypyrimidin-2-yl)phenyl]ethyl}pyridazin-4(1H)-one;
1-(1-ethyl-1H-pyrazol-4-yl)-3-{(1R or S)-1-[3-(5-methoxypyrimidin-2-yl)phenyl]ethyl}pyridazin-4(1H)-one;
rac-1-(1-methyl-1H-pyrazol-4-yl)-3-(1-{3-[5-(piperidin-4-yloxy)pyrimidin-2-yl]phenyl}ethyl)pyridazin-4(1H)-one;
1-(1-ethyl-1H-pyrazol-4-yl)-3-(3-{5-[(4-fluoropiperidin-4-yl)methoxy]pyrimidin-2-yl}benzyl)pyridazin-4(1H)-one;
1-(1-ethyl-1H-pyrazol-4-yl)-3-{3-[5-(piperidin-4-ylmethoxy)pyrimidin-2-yl]benzyl}pyridazin-4(1H)-one;
3-{3-[5-(azetidin-3-yloxy)pyrimidin-2-yl]benzyl}-1-(1-ethyl-1H-pyrazol-4-yl)pyridazin-4(1H)-one;
1-(1-ethyl-1H-pyrazol-4-yl)-3-[(1R or S)-1-(3-{5-[(4-fluoropiperidin-4-yl)methoxy]pyrimidin-2-yl}phenyl)ethyl]pyridazin-4(1H)-one;
1-(1-ethyl-1H-pyrazol-4-yl)-3-((1R or S)-1-{3-[5-(piperidin-4-ylmethoxy)pyrimidin-2-yl]phenyl}ethyl)pyridazin-4(1H)-one;
3-((1R or S)-1-{3-[5-(azetidin-3-yloxy)pyrimidin-2-yl]phenyl}ethyl)-1-(1-ethyl-1H-pyrazol-4-yl)pyridazin-4(1H)-one;
3-[1-(3-{5-[(trans-3-fluoropiperidin-4-yl)oxy]pyrimidin-2-yl}phenyl)ethyl]-1-(1-methyl-1H-pyrazol-4-yl)pyridazin-4(1H)-one;
3-[1-(3-{5-[(cis-3-fluoropiperidin-4-yl)oxy]pyrimidin-2-yl}phenyl)ethyl]-1-(1-methyl-1H-pyrazol-4-yl)pyridazin-4(1H)-one;
1-(3,4-difluorophenyl)-3 (3-{5-[(2R or S)-1,4-dioxan-2-ylmethoxy]pyrimidin-2-yl}benzyl)pyridazin-4(1H)-one;
1-(3,4-difluorophenyl)-3-(3-{5-[(2S or R)-1,4-dioxan-2-ylmethoxy]pyrimidin-2-yl}benzyl)pyridazin-4(1H)-one;
3-(3-{5-[(2R or S)-1,4-dioxan-2-ylmethoxy]pyrimidin-2-yl}benzyl)-1-(1-methyl-1H-pyrazol-4-yl)pyridazin-4(1H)-one;
3-(3-{5-[(2S or R)-1,4-dioxan-2-ylmethoxy]pyrimidin-2-yl}benzyl)-1-(1-methyl-1H-pyrazol-4-yl)pyridazin-4(1H)-one;
1-(3,4-difluorophenyl)-3-(3-{5-[(3R or S)-tetrahydrofuran-3-yloxy]pyrimidin-2-yl}benzyl)pyridazin-4(1H)-one;
1-(3,4-difluorophenyl)-3-(3-{5-[(3S or R)-tetrahydrofuran-3-yloxy]pyrimidin-2-yl}benzyl)pyridazin-4(1H)-one;
1-(3,4-difluorophenyl)-3-(3-{5-[(3R or S)-tetrahydrofuran-3-ylmethoxy]pyrimidin-2-yl}benzyl)pyridazin-4(1H)-one;
1-(3,4-difluorophenyl)-3-(3-{5-[(3S or R)-tetrahydrofuran-3-ylmethoxy]pyrimidin-2-yl}benzyl)pyridazin-4(1H)-one;
1-(1-methyl-1H-pyrazol-4-yl)-3-{3-[5-(pyridin-4-ylmethoxy)pyrimidin-2-yl]benzyl}pyridazin-4(1H)-one;
1-(1-methyl-1H-pyrazol-4-yl)-3-{3-[5-(pyridin-2-ylmethoxy)pyrimidin-2-yl]benzyl}pyridazin-4(1H)-one;
1-(1-methyl-1H-pyrazol-4-yl)-3-{3-[5-pyridin-3-ylmethoxy)pyrimidin-2-yl]benzyl}pyridazin-4(1H)-one;
1-(1-methyl-1H-pyrazol-4-yl)-3-(3-{5-[(1-methyl-1H-1,2,4-triazol-3-yl)methoxy]pyrimidin-2-yl}benzyl)pyridazin-4(1H)-one;
[2-(3-{[1-(1-methyl-1H-pyrazol-4-yl)-4-oxo-1,4-dihydropyridazin-3-yl]methyl}phenyl)pyrimidin-5-yl]oxyacetic acid;
3-{3-[5-(2-hydroxy-2-methylpropoxy)pyrimidin-2-yl]benzyl}-1-(1-methyl-1H-pyrazol-4-yl)pyridazin-4(1H)-one;
1-(1-ethyl-1H-pyrazol-4-yl)-3-{3-[5-(2-hydroxy-2-methylpropoxy)pyrimidin-2-yl]benzyl}pyridazin-4(1H)-one;
1-(3,4-difluorophenyl)-3-{3-[5-(2-hydroxy-2-methylpropoxy)pyrimidin-2-yl]benzyl}pyridazin-4(1H)-one;
3-[3-{3-[5-(2-hydroxy-2-methylpropoxy)pyrimidin-2-yl]benzyl}-4-oxopyridazin-1(4H)-yl]benzonitrile;
3-{3-[5-(2-hydroxy-2-methylpropoxy)pyrimidin-2-yl]benzyl}-1-(3,4,5-trifluorophenyl)pyridazin-4(1H)-one;
1-(3,5-difluorophenyl)-3-{3-[5-(2-hydroxy-2-methylpropoxy)pyrimidin-2-yl]benzyl}pyridazin-4(4H)-one;
rac-1-(1-methyl-1H-pyrazol-4-yl)-3-{3-[5-(3,3,3-trifluoro-2-hydroxypropoxy)pyrimidin-2-yl]benzyl}pyridazin-4(1H)-one;
3-(3-{5-[(4-hydroxytetrahydro-2H-pyran-4-yl)methoxy]pyrimidin-2-yl}benzyl)-1-(1-methyl-1H-pyrazol-4-yl)pyridazin-4(1H)-one;
rac-3-{3-[5-(2-hydroxy-1,2-dimethylpropoxy)pyrimidin-2-yl]benzyl}-1-(1-methyl-1H-pyrazol-4-yl)pyridazin-4(1H)-one;
rac-3-(3-{5-[2-hydroxy-2-(pyridin-4-yl)ethoxy]pyrimidin-2-yl}benzyl)-1-(1-methyl-1H-pyrazol-4-yl)pyridazin-4(1H)-one;
rac-3-{3-[5-(2-hydroxy-3-morpholin-4-ylpropoxy)pyrimidin-2-yl]benzyl}-1-(1-methyl-1H-pyrazol-4-yl)pyridazin-4(1H)-one;
rac-3-{3-[5-(3-fluoro-2-hydroxypropoxy)pyrimidin-2-yl]benzyl}-1-(1-methyl-1H-pyrazol-4-yl)pyridazin-4(1H)-one;
rac-3-{3-[5-(3-ethoxy-2-hydroxypropoxy)pyrimidin-2-yl]benzyl}-1-(1-methyl-1H-pyrazol-4-yl)pyridazin-4(1H)-one;
3-chloro-5-[3-({3-[5-(2-hydroxy-2-methylpropoxy)pyrimidin-2-yl]benzyl}-4-oxopyridazin-1(4H)-yl]benzonitrile;
4-[3-{3-[5-(2-hydroxy-2-methylpropoxy)pyrimidin-2-yl]benzyl}-4-oxopyridazin-1(4H)-yl]benzonitrile;
1-(3,4-difluorophenyl)-3-(3-{5-[(4-hydroxytetrahydro-2H-pyran-4-yl)methoxy]pyrimidin-2-yl}benzyl)pyridazin-4(1H)-one;
3-[3-(5-{[(1R,2R)-2-hydroxy-1-methylpropyl]oxy}pyrimidin-2-yl)benzyl]-1-(1-methyl-1H-pyrazol-4-yl)pyridazin-4(1H)-one;
3-[3-(5-{[(1S,2S)-2-hydroxy-1-methylpropyl]oxy}pyrimidin-2-yl)benzyl]-1-(1-methyl-1H-pyrazol-4-yl)pyridazin-4(1H)-one;
3-[3-(5-{[(R,2S)-2-hydroxy-1-methylpropyl]oxy}pyrimidin-2-yl)benzyl]-1-(1-methyl-1H-pyrazol-4-yl)pyridazin-4(1H)-one;
3-[3-(5-{[(1S,2R)-2-hydroxy-1-methylpropyl]oxy}pyrimidin-2-yl)benzyl]-1-(1-methyl-1H-pyrazol-4-yl)pyridazin-4(1H)-one;
3-[3-(5-{[(1R,2R)-2-hydroxycyclopentyl]oxy}pyrimidin-2-yl)benzyl]-1-(1-methyl-1H-pyrazol-4-yl)pyridazin-4(1H)-one;
3-[3-(5-{[(1S,2S)-2-hydroxycyclopentyl]oxy}pyrimidin-2-yl)benzyl]-1-(1-methyl-1H-pyrazol-4-yl)pyridazin-4(1H)-one;
3-((1S or R)-1-{3-[5-(2-hydroxy-2-methylpropoxy)pyrimidin-2-yl]phenyl}ethyl)-1-(1-methyl-1H-pyrazol-4-yl)pyridazin-4(1H)-one;
3-((1R or S)-1-{3-[5-(2-hydroxy-2-methylpropoxy)pyrimidin-2-yl]phenyl}ethyl)-1-(1-methyl-1H-pyrazol-4-yl)pyridazin-4(1H)-one;
3-{(1R)-1-[3-(5-{[(1R)-2-hydroxy-1,2-dimethylpropyl]oxy}pyrimidin-2-yl)phenyl]ethyl}-1-(1-methyl-1H-pyrazol-4-yl)pyridazin-4(1H)-one;
3-{(1R)-1-[3-(5-({[(S)-2-hydroxy-1,2-dimethylpropyl]oxy}pyrimidin-2-yl)phenyl]ethyl}-1-(1-methyl-1H-pyrazol-4-yl)pyridazin-4(1H)-one;
3-{(1S)-1-[3-(5-{[(1R)-2-hydroxy-1,2-dimethylpropyl]oxy}pyrimidin-2-yl)phenyl]ethyl}-1-(1-methyl-1H-pyrazol-4-yl)pyridazin-4(1H)-one;
3-{(1S)-1-[3-(5-{[(1S)-2-hydroxy-1,2-dimethylpropyl]oxy}pyrimidin-2-yl)phenyl]ethyl}-1-(1-methyl-1H-pyrazol-4-yl)pyridazin-4(1H)-one;
3-{3-[5-(difluoromethoxy)pyrimidin-2-yl]benzyl}-1-(1-methyl-1H-pyrazol-4-yl)pyridazin-4(1H)-one;
3-[3-(5-{[3-(fluoromethyl)oxetan-3-yl]methoxy}pyrimidin-2-yl)benzyl]-1-(1-methyl-1H-pyrazol-4-yl)pyridazin-4(1H)-one;
3-(3-{5-[(4-fluorotetrahydro-2H-pyran-4-yl)methoxy]pyrimidin-2-yl}benzyl)-(1-methyl-1H-pyrazol-4-yl)pyridazin-4(1H)-one;
rac-3-(3-{5-[2-fluoro-2-(pyridin-4-yl)ethoxy]pyrimidin-2-yl}benzyl)-1-(1-methyl-1H-pyrazol-4-yl)pyridazin-4(1H)-one;
rac-3-(3-{5-[(cis-4-fluorotetrahydrofuran-3-yl)oxy]pyrimidin-2-yl}benzyl)-1-(1-methyl-1H-pyrazol-4-yl)pyridazin-4(1H)-one;
3-[3-(5-ethylpyrimidin-2-yl)benzyl]-1-(1-methyl-1H-pyrazol-4-yl)pyridazin-4(1H)-one;
3-[3-(5-butylpyrimidin-2-yl)benzyl]-1-(1-methyl-1H-pyrazol-4-yl)pyridazin-4(1H)-one;
3-[3-(5-cyclopropylpyrimidin-2-yl)benzyl]-1-(1-methyl-1H-pyrazol-4-yl)pyridazin-4(1H)-one;
3-{3-[5-(2-methylpropyl)pyrimidin-2-yl]benzyl}-1-(1-methyl-1H-pyrazol-4-yl)pyridazin-4(1H)-one;
3-{3-[5-(3-hydroxypropyl)pyrimidin-2-yl]benzyl}-1-(1-methyl-1H-pyrazol-4-yl)pyridazin-4(1H)-one;
3-[3-(5-benzylpyrimidin-2-yl)benzyl]-1-(1-methyl-1H-pyrazol-4-yl)pyridazin-4(1H)-one;
1-(1-methyl-1H-pyrazol-4-yl)-3-({3-[5-(2-phenylethyl)pyrimidin-2-yl]benzyl}pyridazin-4(1H)-one;
1-(1-methyl-1H-pyrazol-4-yl)-3-(3-{5-[2-(pyridin-2-yl)ethyl]pyrimidin-2-yl}benzyl)pyridazin-4(1H)-one;
1-(1-methyl-1H-pyrazol-4-yl)-3-{3-[5-(prop-1-en-2-yl)pyrimidin-2-yl]benzyl}pyridazin-4(1H)-one;
1-(1-methyl-1H-pyrazol-4-yl)-3-(3-{5-[(1E)-prop-1-en-1-yl]pyrimidin-2-yl}benzyl)pyridazin-4(1H)-one;
3-(3-{5-[(1E)-3-hydroxy-3-methylbut-1-en-1-yl]pyrimidin-2-yl}benzyl)-1-(1-methyl-1H-pyrazol-4-yl)pyridazin-4(1H)-one;
3-(3-{5-[(1E)-3-methoxyprop-1-en-1-yl]pyrimidin-2-yl}benzyl)-1-(1-methyl-1H-pyrazol-4-yl)pyridazin-4(1H)-one;
3-(3-{5-[(1E)-3-(dimethylamino)prop-1-en-1-yl]pyrimidin-2-yl}benzyl)-1-(1-methyl-1H-pyrazol-4-yl)pyridazin-4(1H)-one;
3-{3-[5-(furan-2-yl)pyrimidin-2-yl]benzyl}-1-(1-methyl-1H-pyrazol-4-yl)pyridazin-4(1H)-one;
3-{(1S or R)-1-[3-(5-cyclopropylpyrimidin-2-yl)phenyl]ethyl}-1-(1-methyl-1H-pyrazol-4-yl)pyridazin-4(1E)-one;
3-{(1R or S)-1-[3-(5-cyclopropylpyrimidin-2-yl)phenyl]ethyl}-1-(1-methyl-1H-pyrazol-4-yl)pyridazin-4(1H)-one;
1-(3,4-difluorophenyl)-3-[3-(5-ethylpyrimidin-2-yl)benzyl]pyridazin-4(1H)-one;
rac-3-{3-[5-(butan-2-yl)pyrimidin-2-yl]benzyl}-1-(1-methyl-1H-pyrazol-4-yl)pyridazin-4(1H)-one;
1-(1-methyl-1H-pyrazol-4-yl)-3-{3-[5-(pyridin-4-yl)pyrimidin-2-yl]benzyl}pyridazin-4(1H)-one;
1-(1-methyl-1H-pyrazol-4-yl)-3-{3-[5-(1H-pyrazol-4-yl)pyrimidin-2-yl]benzyl}pyridazin-4(1H)-one;
3-[3-(5,5′-bipyrimidin-2-yl)benzyl]-1-(1-methyl-1H-pyrazol-4-yl)pyridazin-4(1H)-one;
1-(1-methyl-1H-pyrazol-4-yl)-3-[3-(5-pyridin-3-ylpyrimidin-2-yl)benzyl]pyridazin-4(1H)-one;
5-[2-(3-{[1-(1-methyl-1H-pyrazol-4-yl)-4-oxo-1,4-dihydropyridazin-3-yl]methyl}phenyl)pyrimidin-5-yl]pyridine-2-carbonitrile;
3-{3-[5-(5-fluoropyridin-3-yl)pyrimidin-2-yl]benzyl}-1-(1-methyl-1H-pyrazol-4-yl)pyridazin-4(1H)-one;
3-{3-[5-(3-methoxypyridin-4-yl)pyrimidin-2-yl]benzyl}-1-(1-methyl-1H-pyrazol-4-yl)pyridazin-4(1H)-one;
1-(1-methyl-1H-pyrazol-4-yl)-3-{3-[5-(3-methylpyridin-4-yl)pyrimidin-2-yl]benzyl}pyridazin-4(1H)-one;
3-[3-(2′-amino-5,5′-bipyrimidin-2-yl)benzyl]-1-(1-methyl-1H-pyrazol-4-yl)pyridazin-4(1H)-one;
3-{3-[5-(5-fluoropyridin-2-yl)pyrimidin-2-yl]benzyl}-1-(1-methyl-1H-pyrazol-4-yl)pyridazin-4(1H)-one;
3-({3-[5-(6-aminopyridin-3-yl)pyrimidin-2-yl]benzyl}-1-(1-methyl-1H-pyrazol-4-yl)pyridazin-4(1H)-one;
1-(1-methyl-1H-pyrazol-4-yl)-3-{3-[5-(1H-pyrazol-3-yl)pyrimidin-2-yl]benzyl}pyridazin-4(1H)-one;
1-(1-methyl-1H-pyrazol-4-yl)-3-{3-[5-(1-methyl-1H-pyrazol-3-yl)pyrimidin-2-yl]benzyl}pyridazin-4(1H)-one;
1-(1-methyl-1H-pyrazol-4-yl)-3-{3-[5-(1,3-thiazol-4-yl)pyrimidin-2-yl]benzyl}pyridazin-4(1H)-one;
3-[3-(5-isoxazol-4-ylpyrimidin-2-yl)benzyl]-1-(1-methyl-1H-pyrazol-4-yl)pyridazin-4(1H)-one;
3-{3-[5-(3,5-dimethylisoxazol-4-yl)pyrimidin-2-yl]benzyl}-1-(1-methyl-1H-pyrazol-4-yl)pyridazin-4(1H)-one;
1-(1-methyl-1H-pyrazol-4-yl)-3-[3-(5-pyridazin-4-ylpyrimidin-2-yl)benzyl]pyridazin-4(1H)-one;
1-(1-methyl-1H-pyrazol-4-yl)-3-{3-[5-(morpholin-4-ylmethyl)pyrimidin-2-yl]benzyl}pyridazin-4(1H)-one;
3-(3-{5-[(methylamino)methyl]pyrimidin-2-yl}benzyl)-1-(1-methyl-1H-pyrazol-4-yl)pyridazin-4(1H)-one;
1-(1-methyl-1H-pyrazol-4-yl)-3-{3-[5-(thiomorpholin-4-ylmethyl)pyrimidin-2-yl]benzyl}pyridazin-4(1H)-one;
1-(1-methyl-1H-pyrazol-4-yl)-3-{3-[5-(pyrrolidin-1-ylmethyl)pyrimidin-2-yl]benzyl}pyridazin-4(1H)-one;
3-(3-{5-[(dimethylamino)methyl]pyrimidin-2-yl}benzyl)-1-(1-methyl-1H-pyrazol-4-yl)pyridazin-4(1H)-one;
rac-3-(3-{5-[(3-fluoropyrrolidin-1-yl)methyl]pyrimidin-2-yl}benzyl)-1-(1-methyl-1H-pyrazol-4-yl)pyridazin-4(1H)-one;
3-(3-{5-[(cyclohexylamino)methyl]pyrimidin-2-yl}benzyl)-1-(1-methyl-1H-pyrazol-4-yl)pyridazin-4(1H)-one;
1-(1-methyl-1H-pyrazol-4-yl)-3-{3-[5-(1-oxetan-3-yl-1H-pyrazol-4-yl)pyrimidin-2-yl]benzyl}pyridazin-4(1H)-one;
3-(3-{5-[(4-aminopiperidin-1-yl)methyl]pyrimidin-2-yl}benzyl)-1-(1-methyl-1H-pyrazol-4-yl)pyridazin-4(1H)-one;
1-(1-methyl-1H-pyrazol-4-yl)-3-{3-[5-(1-oxetan-3-yl-1H-pyrazol-4-yl)pyrimidin-2-yl]benzyl}pyridazin-4(1H)-one;
1-(1 methyl-1H-pyrazol-4-yl)-3-{3-[5-(propan-2-yl)pyrimidin-2-yl]benzyl}pyridazin-4(1H)-one;
3-{3-[5-(3-hydroxy-3-methylbutyl)pyrimidin-2-yl]benzyl}-1-(1-methyl-1H-pyrazol-4-yl)pyridazin-4(1H)-one;
3-(3-{5-[3-(dimethylamino)propyl]pyrimidin-2-yl}benzyl)-1-(1-methyl-1H-pyrazol-4-yl)pyridazin-4(1H)-one;
3-{3-[5-(3-methoxypropyl)pyrimidin-2-yl]benzyl}-1-(1-methyl-1H-pyrazol-4-yl)pyridazin-4(1H)-one;
rac-3-({3-[5-(3-hydroxypyrrolidin-1-yl)pyrimidin-2-yl]benzyl}-(1-methyl-1H-pyrazol-4-yl)pyridazin-4(1H)-one;
1-(1-methyl-1H-pyrazol-4-yl)-3-[3-(5-piperidin-1-yl)pyrimidin-2-yl)benzyl]pyridazin-4(1H)-one;
3-{3-[5-(4-hydroxypiperidin-1-yl)pyrimidin-2-yl]benzyl}-1-(1-methyl-1H-pyrazol-4-yl)pyridazin-4(1H)-one;
1-(1-methyl-1H-pyrazol-4-yl)-3-({3-[5-(octahydroisoquinolin-2(1H)-yl)pyrimidin-2-yl]benzyl}pyridazin-4(1H)-one;
3-(3-{5-[4-(dimethylamino)piperidin-1-yl]pyrimidin-2-yl}benzyl)-1-(1-methyl-1H-pyrazol-4-yl)pyridazin-4(1H)-one;
1-[2-(3-({[1-(1-methyl-1H-pyrazol-4-yl)-4-oxo-1,4-dihydropyridazin-3-yl]methyl}phenyl)pyrimidin-5-yl]piperidine-4-carboxamide;
rac-1-[2-(3-{[1-(1-methyl-1H-pyrazol-4-yl)-4-oxo-1,4-dihydropyridazin-3-yl]methyl}phenyl)pyrimidin-5-yl]piperidine-3-carbonitrile;
3-{3-[5-(3,3-difluoropyrrolidin-1-yl)pyrimidin-2-yl]benzyl}-1-(1-methyl-1H-pyrazol-4-yl)pyridazin-4(1H)-one;
3-{3-[5-(1,1-dioxidothiomorpholin-4-yl)pyrimidin-2-yl]benzyl}-1-(1-methyl-1H-pyrazol-4-yl)pyridazin-4(1H)-one;
rac-3-(3-{5-[3-(methoxymethyl)piperidin-1-yl]pyrimidin-2-yl}benzyl)-1-(1-methyl-1H-pyrazol-4-yl)pyridazin-4(1H)-one;
rac-3-{3-[5-(3-methylmorpholin-4-yl)pyrimidin-2-yl]benzyl}-1-(1-methyl-1H-pyrazol-4-yl)pyridazin-4(1H)-one;
1-(1-methyl-1H-pyrazol-4-yl)-3-{3-[5-(propylamino)pyrimidin-2-yl]benzyl}pyridazin-4(1H)-one;
3-{3-[5-(ethylamino)pyrimidin-2-yl]benzyl}-1-(1-methyl-1H-pyrazol-4-yl)pyridazin-4(1H)-one;
3-(3-{5-[(2-methoxyethyl)amino]pyrimidin-2-yl}benzyl)-1-(1-methyl-1H-pyrazol-4-yl)pyridazin-4(1H)-one;
3-(3-{5-[(2-ethoxyethyl)amino]pyrimidin-2-yl}benzyl)-1-(1-methyl-1H-pyrazol-4-yl)pyridazin-4(1H)-one;
1-(1-methyl-1H-pyrazol-4-yl)-3-(3-{5-[(tetrahydrofuran-3-ylmethyl)amino]pyrimidin-2-yl}benzyl)pyridazin-4(1H)-one;
3-(3-{5-[methyl(propyl)amino]pyrimidin-2-yl}benzyl)-1-(1-methyl-1H-pyrazol-4-yl)pyridazin-4(1H)-one;
3-(3-{5-[(2-methoxyethyl)(methyl)amino]pyrimidin-2-yl}benzyl)-1-(1-methyl-1H-pyrazol-4-yl)pyridazin-4(1H)-one;
3-methoxy-N-[2-(3-{[1-(1-methyl-1H-pyrazol-4-yl)-4-oxo-1,4-dihydropyridazin-3-yl]methyl}phenyl)pyrimidin-5-yl]propanamide;
N-[2-(3-{[1-(1-methyl-1H-pyrazol-4-yl)-4-oxo-1,4-dihydropyridazin-3-yl]methyl}phenyl)pyrimidin-5-yl]-2-(tetrahydro-2H-pyran-4-yl)acetamide;
N-[2-(3-{[1-(1-methyl-1H-pyrazol-4-yl)-yl)-4-oxo-1,4-dihydropyridazin-3-yl]methyl}=phenyl)pyrimidin-5-yl]propanamide;
2-methoxy-N-[2-(3-{[1-(1-methyl-1H-pyrazol-4-yl)-4-oxo-1,4-dihydropyridazin-3-yl]methyl}phenyl)pyrimidin-5-yl]acetamide;
rac-N-[2-(3-{[1-(1-methyl-1H-pyrazol-4-yl)-4-oxo-1,4-dihydropyridazin-3-yl]methyl}phenyl)pyrimidin-5-yl]tetrahydrofuran-2-carboxamide;
N-[2-(3-{[1-(1-methyl-1H-pyrazol-4-yl)-4-oxo-1,4-dihydropyridazin-3-yl]methyl}phenyl)pyrimidin-5-yl]-2-(2-oxopyrrolidin-1-yl)acetamide;
rac-N-[2-(3-{[1-(1-methyl-1H-pyrazol-4-yl)-4-oxo-1,4-dihydropyridazin-3-yl]methyl}phenyl)pyrimidin-5-yl]-2-(tetrahydrofuran-2-yl)acetamide;
3-[3-(5-bromopyrimidin-2-yl)benzyl]-1-(1-methyl-1H-pyrazol-4-yl)pyridazin-4(1H)-one;
3-{3-[5-(4-methylpiperazin-1-yl)pyrimidin-2-yl]benzyl}-1-(1-methyl-1H-pyrazol-4-yl)pyridazin-4(1H)-one;
rac-3-{3-[5-(3-fluoropiperidin-1-yl)pyrimidin-2-yl]benzyl}-1-(1-methyl-1H-pyrazol-4-yl)pyridazin-4(1H)-one;
rac-3-{3-[5-(3-methylpiperidin-1-yl)pyrimidin-2-yl]benzyl}-1-(1-methyl-1H-pyrazol-4-yl)pyridazin-4(1H)-one;
1-(1-methyl-1H-pyrazol-4-yl)-3-[3-(5-pyrrolidin-1-ylpyrimidin-2-yl)benzyl]pyridazin-4(1H)-one;
tert-butyl 4-[2-(3-{([1-(1-methyl-1H-pyrazol-4-yl)-4-oxo-1,4-dihydropyridazin-3-yl]methyl}phenyl)pyrimidin-5-yl]piperazine-1-carboxylate;
1-(1-methyl-1H-pyrazol-4-yl)-3-[3-(5-morpholin-4-ylpyrimidin-2-yl)benzyl]pyridazin-4(1H)-one;
1-(1-methyl-1H-pyrazol-4-yl)-3-{3-[5-(4H-1,2,4-triazol-4-yl)pyrimidin-2-yl]benzyl}pyridazin-4(1H)-one;
rac-2-(3-{[1-(1-methyl-1H-pyrazol-4-yl)-4-oxo-1,4-dihydropyridazin-3-yl]methyl}phenyl)-N-(tetrahydrofuran-3-ylmethyl)pyrimidine-5-carboxamide;
rac-N-(1,4-dioxan-2-ylmethyl)-2-(3-{[1-(1-methyl-1H-pyrazol-4-yl)-4-oxo-1,4-dihydropyridazin-3-yl]methyl}phenyl)pyrimidine-5-carboxamide;
2-(3-{[1-(1-methyl-1H-pyrazol-4-yl)-4-oxo-1,4-dihydropyridazin-3-yl]methyl}phenyl)-N-(2-morpholin-4-ylethyl)pyrimidine-5-carboxamide;
rac-2-(3-{[1-(1-methyl-1H-pyrazol-4-yl)-4-oxo-1,4-dihydropyridazin-3-yl]methyl}phenyl)-N-(tetrahydro-2H-pyran-3-ylmethyl)pyrimidine-5-carboxamide;
N-[3-(4-methylpiperazin-1-yl)propyl]-2-(3-{[1-(1-methyl-1H-pyrazol-4-yl)-4-oxo-1,4-dihydropyridazin-3-yl]methyl}phenyl)pyrimidine-5-carboxamide;
N-(2-methylpropyl)-2-(3-{[1-(1-methyl-1H-pyrazol-4-yl)-4-oxo-1,4-dihydropyridazin-3-yl]methyl}phenyl)pyrimidine-5-carboxamide;
2-(3-({[1-(1-methyl-1H-pyrazol-4-yl)-4-oxo-1,4-dihydropyridazin-3-yl]methyl}phenyl)-N-(2,2,2-trifluoroethyl)pyrimidine-5-carboxamide;
2-(3-{[1-(1-methyl-1H-pyrazol-4-yl)-4-oxo-1,4-dihydropyridazin-3-yl]methyl}phenyl)-N-(3-morpholin-4-ylpropyl)pyrimidine-5-carboxamide;
rac-2-(3-{[1-(1-methyl-1H-pyrazol-4-yl)-4-oxo-1,4-dihydropyridazin-3-yl]methyl}phenyl)-N-(tetrahydrofuran-2-ylmethyl)pyrimidine-5-carboxamide;
N-ethyl-2-(3-{[1-(1-methyl-1H-pyrazol-4-yl)-4-oxo-1,4-dihydropyridazin-3-yl]methyl}phenyl)pyrimidine-5-carboxamide;
N-methyl-2-(3-{[1-(1-methyl-1H-pyrazol-4-yl)-4-oxo-1,4-dihydropyridazin-3-yl]methyl}phenyl)pyrimidine-5-carboxamide;
1-ethyl-3-{3-[(4-oxo-1-phenyl-1,4-dihydropyridazin-3-yl)methyl]phenyl}urea;
1-methyl-3-(3-{[1-(1-methyl-1H-pyrazol-4-yl)-4-oxo-1,4-dihydropyridazin-3-yl]methyl}phenyl)urea;
1-ethyl-3-(3-{[1-(1-methyl-1H-pyrazol-4-yl)-4-oxo-1,4-dihydropyridazin-3-yl]methyl}phenyl)urea;
1-(3-{[1-(1-methyl-1H-pyrazol-4-yl)-4-oxo-1,4-dihydropyridazin-3-yl]methyl}phenyl)-3-propylurea;
1-benzyl-3-(3-{[1-(1-methyl-1H-pyrazol-4-yl)-4-oxo-1,4-dihydropyridazin-3-yl]methyl}phenyl)urea;
1-(2-methylpropyl)-3-(3-{[1-(1-methyl-1H-pyrazol-4-yl)-4-oxo-1,4-dihydropyridazin-3-yl]methyl}phenyl)urea;
1-cyclopropyl-3-(3-{[1-(1-methyl-1H-pyrazol-4-yl)-4-oxo-1,4-dihydropyridazin-3-yl]methyl}phenyl)urea;
1-(2-methoxyethyl)-3-(3-{[1-(1-methyl-1H-pyrazol-4-yl)-4-oxo-1,4-dihydropyridazin-3-yl]methyl}phenyl)urea;
1-butyl-3-(3-{[1-(1-methyl-1H-pyrazol-4-yl)-4-oxo-1,4-dihydropyridazin-3-yl]methyl}phenyl)urea;
1-(4-methoxybenzyl)-3-(3-{[1-(1-methyl-1H-pyrazol-4-yl)-4-oxo-1,4-dihydropyridazin-3-yl]methyl}phenyl)urea;
1-(3-{[1-(3,4-difluorophenyl)-4-oxo-1,4-dihydropyridazin-3-yl]mmethyl}phenyl)-3-(2-morpholin-4-ylethyl)urea;
methyl(3-{[1-(1-methyl-1H-pyrazol-4-yl)-4-oxo-1,4-dihydropyridazin-3-yl]methyl}phenyl)carbamate;
benzyl (3-{[1-(1-methyl-1H-pyrazol-4-yl)-4-oxo-1,4-dihydropyridazin-3-yl]methyl}phenyl)carbamate;
2-fluoroethyl(3-{[1-(1-methyl-1H-pyrazol-4-yl)-4-oxo-1,4-dihydropyridazin-3-yl]methyl}phenyl)carbamate;
butyl (3-{[1-(1-methyl-TH-pyrazol-4-yl)-4-oxo-1,4-dihydropyridazin-3-yl]methyl}phenyl)carbamate;
2,2-dimethylpropyl (3-{[1-(1-methyl-1H-pyrazol-4-yl)-4-oxo-1,4-dihydropyridazin-3-yl]methyl}phenyl)carbamate;
2-methoxyethyl(3-{[1-(3-cyanophenyl)-4-oxo-1,4-dihydropyridazin-3-yl]methyl}phenyl)carbamate;
2-methoxyethyl(3-{[4-oxo-1-(3,4,5-trifluorophenyl)-1,4-dihydropyridazin-3-yl]methyl}phenyl)carbamate;
ethyl(3-{[1-(3,4-difluorophenyl)-4-oxo-1,4-dihydropyridazin-3-yl]methyl}phenyl)carbamate;
2-methylpropyl (3-{[1-(3,4-difluorophenyl)-4-oxo-1,4-dihydropyridazin-3-yl]methyl}phenyl)carbamate;
2-methoxyethyl(3-{[1-(3,4-difluorophenyl)-4-oxo-1,4-dihydropyridazin-3-yl]methyl}phenyl)carbamate;
2-methoxyethyl(3-{[1-(3,5-difluorophenyl)-4-oxo-1,4-dihydropyridazin-3-yl]methyl}phenyl)carbamate;
2-methoxyethyl(3-{[1-(4-cyanophenyl)-4-oxo-1,4-dihydropyridazin-3-yl]methyl}phenyl)carbamate;
2-methoxyethyl(3-{[1-(3-chloro-5-fluorophenyl)-4-oxo-1,4-dihydropyridazin-3-yl]methyl}phenyl)carbamate;
2-(1H-imidazol-1-yl)ethyl(3-{[1-(1-methyl-1H-pyrazol-4-yl)-4-oxo-1,4-dihydropyridazin-3-yl]methyl}phenyl)carbamate;
3-(4-methylpiperazin-1-yl)propyl (3-{[4-oxo-1-(3,4,5-trifluorophenyl)-1,4-dihydropyridazin-3-yl]methyl}phenyl)carbamate;
1-(2-{[(3-{[4-oxo-1-(3,4,5-trifluorophenyl)-1,4-dihydropyridazin-3-yl]methyl}phenyl)carbamoyl]oxy}ethyl)piperidine-4-carboxylic acid;
rac-1,4-dioxan-2-ylmethyl(3-{[4-oxo-1-(3,4,5-trifluorophenyl)-1,4-dihydropyridazin-3-yl]methyl}phenyl)carbamate;
3-hydroxy-3-methylbutyl (3-{[4-oxo-1-(3,4,5-trifluorophenyl)-1,4-dihydropyridazin-3-yl]methyl}phenyl)carbamate;
2-(1,1-dioxidothiomorpholin-4-yl)ethyl(3-{[4-oxo-1-(3,4,5-trifluorophenyl)-1,4-dihydropyridazin-3-yl]methyl}phenyl)carbamate;
2-(4-methylpiperazin-1-yl)ethyl(3-{[4-oxo-1-(3,4,5-trifluorophenyl)-1,4-dihydropyridazin-3-yl]methyl}phenyl)carbamate
2-(1,1-dioxidothiomorpholin-4-yl)ethyl(3-{[1-(3,5-difluorophenyl)-4-oxo-1,4-dihydropyridazin-3-yl]methyl}phenyl)carbamate;
3-(4-methylpiperazin-1-yl)propyl (3-{[1-(3,5-difluorophenyl)-4-oxo-1,4-dihydropyridazin-3-yl]methyl}phenyl)carbamate;
2-(4-methylpiperazin-1-yl)ethyl(3-{[1-(3,5-difluorophenyl)-4-oxo-1,4-dihydropyridazin-3-yl]methyl}phenyl)carbamate;
rac-1,4-dioxan-2-ylmethyl(3-{[1-(3,5-difluorophenyl)-4-oxo-1,4-dihydropyridazin-3-yl]methyl}phenyl)carbamate;
3-morpholin-4-ylpropyl (3-{[1-(3,5-difluorophenyl)-4-oxo-1,4-dihydropyridazin-3-yl]methyl}phenyl)carbamate;
3-(1,1-dioxidothiomorpholin-4-yl)propyl (3-{[1-(3,5-difluorophenyl)-4-oxo-1,4-dihydropyridazin-3-yl-]methyl}phenyl)carbamate;
2-morpholin-4-ylethyl(3-{[1-(3,5-difluorophenyl)-4-oxo-1,4-dihydropyridazin-3-yl]methyl}phenyl)carbamate;
2-(3,3-difluoropyrrolidin-1-yl)ethyl (3-{[1-(1-methyl-1H-pyrazol-4-yl)-4-oxo-1,4-dihydropyridazin-3-yl]methyl}phenyl)carbamate;
2,2-difluoro-3-morpholin-4-ylpropyl (3-{[1-(1-methyl-1H-pyrazol-4-yl)-4-oxo-1,4-dihydropyridazin-3-yl]methyl}phenyl)carbamate;
3-hydroxy-3-methylbutyl (3-{[1-(1-methyl-1H-pyrazol-4-yl)-4-oxo-1,4-dihydropyridazin-3-yl]methyl}phenyl)carbamate;
tetrahydro-2H-pyran-4-ylmethyl(3-{[1-(3,4-difluorophenyl)-4-oxo-1,4-dihydropyridazin-3-yl]methyl}phenyl)carbamate;
2-(1,1-dioxidothiomorpholin-4-yl)ethyl(3-{[1-(3,4-difluorophenyl)-4-oxo-1,4-dihydropyridazin-3-yl]methyl}phenyl)carbamate;
2-(3-oxopiperazin-1-yl)ethyl(3-{[1-(3,4-difluorophenyl)-4-oxo-1,4-dihydropyridazin-3-yl]methyl}phenyl)carbamate;
2-(4-methylpiperazin-1-yl)ethyl(3-({[1-(1-methyl-1H-pyrazol-4-yl)-4-oxo-1,4-dihydropyridazin-3-yl]methyl}phenyl)carbamate;
2-morpholin-4-ylethyl(3-{[1-(1-methyl-1H-pyrazol-4-yl)-4-oxo-1,4-dihydropyridazin-3-yl]methyl}phenyl)carbamate;
2-(1H-imidazol-1-yl)ethyl(3-{[1-(1-methyl-1H-pyrazol-4-yl)-4-oxo-1,4-dihydropyridazin-3-yl]methyl}phenyl)carbamate;
2-(2-oxopyrrolidin-1-yl)ethyl(3-{[1-(1-methyl-1H-pyrazol-4-yl)-4-oxo-1,4-dihydropyridazin-3-yl]methyl}phenyl)carbamate;
2-(1H-1,2,4-triazol-1-yl)ethyl(3-{[1-(1-methyl-1H-pyrazol-4-yl)-4-oxo-1,4-dihydropyridazin-3-yl]methyl}phenyl)carbamate;
2-(3-oxomorpholin-4-yl)ethyl(3-{[1-(1-methyl-1H-pyrazol-4-yl)-4-oxo-1,4-dihydropyridazin-3-yl]methyl}phenyl)carbamate;
3-(4-methylpiperazin-1-yl)propyl (3-{[1-(1-methyl-1H-pyrazol-4-yl)-4-oxo-1,4-dihydropyridazin-3-yl]methyl}phenyl)carbamate;
3-morpholin-4-ylpropyl (3-{[1-(1-methyl-1H-pyrazol-4-yl)-4-oxo-1,4-dihydropyridazin-3-yl]methyl}phenyl)carbamate;
cyclobutylmethyl(3-{[1-(1-methyl-1H-pyrazol-4-yl)-4-oxo-1,4-dihydropyridazin-3-yl]methyl}phenyl)carbamate;
cyclopentylmethyl(3-{[1-(1-methyl-1H-pyrazol-4-yl)-4-oxo-1,4-dihydropyridazin-3-yl]methyl}phenyl)carbamate;
cyclohexylmethyl(3-{[1-(1-methyl-1H-pyrazol-4-yl)-4-oxo-1,4-dihydropyridazin-3-yl]methyl}phenyl)carbamate;
tetrahydro-2H-pyran-4-ylmethyl(3-{[1-(1-methyl-1H-pyrazol-4-yl)-4-oxo-1,4-dihydropyridazin-3-yl]methyl}phenyl)carbamate
rac-tetrahydrofuran-3-ylmethyl(3-{[1-(1-methyl-1H-pyrazol-4-yl)-4-oxo-1,4-dihydropyridazin-3-yl]methyl}phenyl)carbamate;
(3-methyloxetan-3-yl)methyl(3-{[1-(1-methyl-1H-pyrazol-4-yl)-4-oxo-1,4-dihydropyridazin-3-yl]methyl}phenyl)carbamate;
2,2,2-trifluoroethyl(3-{[1-(1-methyl-1H-pyrazol-4-yl)-4-oxo-1,4-dihydropyridazin-3-yl]methyl}phenyl)carbamate;
3-(dimethylamino)-3-oxopropyl (3-{[1-(1-methyl-1H-pyrazol-4-yl)-4-oxo-1,4-dihydropyridazin-3-yl]methyl}phenyl)carbamate;
2-(dimethylamino)ethyl(3-{[4-oxo-1-(3,4,5-trifluorophenyl)-1,4-dihydropyridazin-3-yl]methyl}phenyl)carbamate;
2-(H-imidazol-1-yl)ethyl(3-{[4-oxo-1-(3,4,5-trifluorophenyl)-1,4-dihydropyridazin-3-yl]methyl}phenyl)carbamate;
3-(1H-pyrrol-1-yl)propyl (3-{[4-oxo-1-(3,4,5-trifluorophenyl)-1,4-dihydropyridazin-3-yl]methyl}phenyl)carbamate;
2-(2-oxopyrrolidin-1-yl)ethyl(3-{[4-oxo-1-(3,4,5-trifluorophenyl)-1,4-dihydropyridazin-3-yl]methyl}phenyl)carbamate;
2-[methyl(phenyl)amino]ethyl(3-{[4-oxo-1-(3,4,5-trifluorophenyl)-1,4-dihydropyridazin-3-yl]methyl}phenyl)carbamate;
3-(2-oxopyrrolidin-1-yl)propyl (3-{[4-oxo-1-(3,4,5-trifluorophenyl)-1,4-dihydropyridazin-3-yl]methyl}phenyl)carbamate;
2-(1H-1,2,4-triazol-1-yl)ethyl(3-{[4-oxo-1-(3,4,5-trifluorophenyl)-1,4-dihydropyridazin-3-yl]methyl}phenyl)carbamate;
3-(4-methylpiperidin-1-yl)propyl (3-{[4-oxo-1-(3,4,5-trifluorophenyl)-1,4-dihydropyridazin-3-yl]methyl}phenyl)carbamate;
3-pyrrolidin-1-ylpropyl (3-{[4-oxo-1-(3,4,5-trifluorophenyl)-1,4-dihydropyridazin-3-yl]methyl}phenyl)carbamate;
cyclobutylmethyl(3-{[4-oxo-1-(3,4,5-trifluorophenyl)-1,4-dihydropyridazin-3-yl]methyl}phenyl)carbamate;
cyclopentylmethyl(3-{[4-oxo-1-(3,4,5-trifluorophenyl)-1,4-dihydropyridazin-3-yl]methyl}phenyl)carbamate;
cyclohexylmethyl(3-{[4-oxo-1-(3,4,5-trifluorophenyl)-1,4-dihydropyridazin-3-yl]methyl}phenyl)carbamate;
tetrahydro-2H-pyran-4-ylmethyl(3-{[4-oxo-1-(3,4,5-trifluorophenyl)-1,4-dihydropyridazin-3-yl]methyl}phenyl)carbamate;
rac-tetrahydrofuran-3-ylmethyl(3-{[4-oxo-1-(3,4,5-trifluorophenyl)-1,4-dihydropyridazin-3-yl]methyl}phenyl)carbamate;
(3-methyloxetan-3-yl)methyl(3-{[4-oxo-1-(3,4,5-trifluorophenyl)-1,4-dihydropyridazin-3-yl]methyl}phenyl)carbamate;
3-(dimethylamino)-3-oxopropyl (3-{[4-oxo-1-(3,4,5-trifluorophenyl)-1,4-dihydropyridazin-3-yl]methyl}phenyl)carbamate;
rac-tetrahydrofuran-2-ylmethyl(3-{[1-(1-methyl-1H-pyrazol-4-yl)-4-oxo-1,4-dihydropyridazin-3-yl]methyl}phenyl)carbamate;
rac-tetrahydro-2H-pyran-2-ylmethyl(3-{[1-(1-methyl-1H-pyrazol-4-yl)-4-oxo-1,4-dihydropyridazin-3-yl]methyl}phenyl)carbamate;
3,3,3-trifluoropropyl (3-{[1-(1-methyl-1H-pyrazol-4-yl)-4-oxo-1,4-dihydropyridazin-3-yl]methyl}phenyl)carbamate;
2-(tetrahydro-2H-pyran-4-yl)ethyl (3-{[1-(1-methyl-1H-pyrazol-4-yl)-4-oxo-1,4-dihydropyridazin-3-yl]methyl}phenyl)carbamate;
3-(1,1-dioxidothiomorpholin-4-yl)propyl (3-{[1-(1-methyl-1H-pyrazol-4-yl)-4-oxo-1,4-dihydropyridazin-3-yl]methyl}phenyl)carbamate;
2-(1,1-dioxidothiomorpholin-4-yl)ethyl (3-{[1-(1-methyl-1H-pyrazol-4-yl)-4-oxo-1,4-dihydropyridazin-3-yl]methyl}phenyl)carbamate;
rac-1,4-dioxan-2-ylmethyl(3-{[t-(1-methyl-1H-pyrazol-4-yl)-4-oxo-1,4-dihydropyridazin-3-yl]methyl}phenyl)carbamate;
rac-tetrahydro-2H-pyran-3-ylmethyl(3-{[1-(1-methyl-1H-pyrazol-4-yl)-4-oxo-1,4-dihydropyridazin-3-yl]methyl}phenyl)carbamate;
rac-[1-(2,2,2-trifluoro-1-methylethyl)azetidin-3-yl]methyl(3-{[1-(1-methyl-1H-pyrazol-4-yl)-4-oxo-1,4-dihydropyridazin-3-yl]methyl}phenyl)carbamate;
3-(diethylamino)propyl (3-{[1-(1-methyl-1H-pyrazol-4-yl)-4-oxo-1,4-dihydropyridazin-3-yl]methyl}phenyl)carbamate;
4-hydroxybutyl (3-{[1-(1-methyl-1H-pyrazol-4-yl)-4-oxo-1,4-dihydropyridazin-3-yl]methyl}phenyl)carbamate;
rac-2-methylbutyl (3-{[1-(1-methyl-1H-pyrazol-4-yl)-4-oxo-1,4-dihydropyridazin-3-yl]methyl}phenyl)carbamate;
(2-methylcyclopropyl)methyl(3-{[1-(1-methyl-1H-pyrazol-4-yl)-4-oxo-1,4-dihydropyridazin-3-yl]methyl}phenyl)carbamate;
3-methoxypropyl (3-{[1-(1-methyl-1H-pyrazol-4-yl)-4-oxo-1,4-dihydropyridazin-3-yl]methyl}phenyl)carbamate;
2,2-difluoroethyl(3-{[(1-(1-methyl-1H-pyrazol-4-yl)-4-oxo-1,4-dihydropyridazin-3-yl]methyl}phenyl)carbamate;
2-(cyclohexyloxy)ethyl(3-{[1-(1-methyl-1H-pyrazol-4-yl)-4-oxo-1,4-dihydropyridazin-3-yl]methyl}phenyl)carbamate;
rac-oxetan-2-ylmethyl(3-{[1-(1-methyl-1H-pyrazol-4-yl)-4-oxo-1,4-dihydropyridazin-3-yl]methyl}phenyl)carbamate;
tetrahydro-2H-pyran-4-ylmethyl(3-{[1-(3-cyanophenyl)-4-oxo-1,4-dihydropyridazin-3-yl]methyl}phenyl)carbamate;
propyl (3-{[1-(3-cyanophenyl)-4-oxo-1,4-dihydropyridazin-3-yl]methyl}phenyl)carbamate;
rac-2-methoxybutyl (3-{[1-(3-cyanophenyl)-4-oxo-1,4-dihydropyridazin-3-yl]methyl}phenyl)carbamate;
2-(2-oxopyrrolidin-1-yl)ethyl(3-{[1-(3-cyanophenyl)-4-oxo-1,4-dihydropyridazin-3-yl]methyl}phenyl)carbamate;
rac-tetrahydrofuran-3-ylmethyl(3-{[1-(3-cyanophenyl)-4-oxo-1,4-dihydropyridazin-3-yl]methyl}phenyl)carbamate;
2-(3-oxomorpholin-4-yl)ethyl(3-{[1-(3-cyanophenyl)-4-oxo-1,4-dihydropyridazin-3-yl]methyl}phenyl)carbamate;
rac-[1-(2-methoxyethyl)pyrrolidin-3-yl]methyl(3-{[1-(3-cyanophenyl)-4-oxo-1,4-dihydropyridazin-3-yl]methyl}phenyl)carbamate;
2-(2,2,2-trifluoroethoxy)ethyl(3-{[1-(3-cyanophenyl)-4-oxo-1,4-dihydropyridazin-3-yl]methyl}phenyl)carbamate;
2-(1H-1,2,4-triazol-1-yl)ethyl(3-{[1-(3-cyanophenyl)-4-oxo-1,4-dihydropyridazin-3-yl]methyl}phenyl)carbamate;
3-(dimethylamino)-3-oxopropyl (3-{[1-(3-cyanophenyl)-4-oxo-1,4-dihydropyridazin-3-yl]methyl}phenyl)carbamate;
3-(dimethylamino)-3-oxopropyl (3-{[1-(3,5-difluorophenyl)-4-oxo-1,4-dihydropyridazin-3-yl]methyl}phenyl)carbamate;
propyl (3-{[1-(3,5-difluorophenyl)-4-oxo-1,4-dihydropyridazin-3-yl]methyl}phenyl)carbamate;
rac-2-methoxybutyl (3-{[1-(3,5-difluorophenyl)-4-oxo-1,4-dihydropyridazin-3-yl]methyl}phenyl)carbamate;
2-(2-oxopyrrolidin-1-yl)ethyl(3-{[1-(3,5-difluorophenyl)-4-oxo-1,4-dihydropyridazin-3-yl]methyl}phenyl)carbamate;
rac-tetrahydrofuran-3-ylmethyl(3-{[1-(3,5-difluorophenyl)-4-oxo-1,4-dihydropyridazin-3-yl]methyl}phenyl)carbamate;
rac-[1-(2-methoxyethyl)pyrrolidin-3-yl]methyl(3-{[1-(3,5-difluorophenyl)-4-oxo-1,4-dihydropyridazin-3-yl]methyl}phenyl)carbamate;
2-(2,2,2-trifluoroethoxy)ethyl (3-{[1-(3,5-difluorophenyl)-4-oxo-1,4-dihydropyridazin-3-yl]methyl}phenyl)carbamate;
2-(1,1-dioxidothiomorpholin-4-yl)ethyl(3-{[1-(3-cyanophenyl)-4-oxo-1,4-dihydropyridazin-3-yl]methyl}phenyl)carbamate;
2-morpholin-4-ylethyl(3-{[1-(3-cyanophenyl)-4-oxo-1,4-dihydropyridazin-3-yl]methyl}phenyl)carbamate;
2-(tetrahydro-2H-pyran-4-yl)ethyl(3-{[1-(3-cyanophenyl)-4-oxo-1,4-dihydropyridazin-3-yl]methyl}phenyl)carbamate;
2-(2-methoxyethoxy)ethyl(3-{[4-oxo-1-(3,4,5-trifluorophenyl)-1,4-dihydropyridazin-3-yl]methyl}phenyl)carbamate;
2-(2-methoxyethoxy)ethyl(3-{[1-(3-cyanophenyl)-4-oxo-1,4-dihydropyridazin-3-yl]methyl}phenyl)carbamate;
rac-1,4-dioxan-2-ylmethyl(3-{[1-(3-cyanophenyl)-4-oxo-1,4-dihydropyridazin-3-yl]methyl}phenyl)carbamate;
2-(2-methoxyethoxy)ethyl(3-{[1-(3,4-difluorophenyl)-4-oxo-1,4-dihydropyridazin-3-yl]methyl}phenyl)carbamate;
rac-1,4-dioxan-2-ylmethyl(3-{[1-(3,4-difluorophenyl)-4-oxo-1,4-dihydropyridazin-3-yl]methyl}phenyl)carbamate;
2-(3-oxopiperazin-1-yl)ethyl(3-{[1-(3,5-difluorophenyl)-4-oxo-1,4-dihydropyridazin-3-yl]methyl}phenyl)carbamate;
2-(3-oxomorpholin-4-yl)ethyl(3-{[1-(3,5-difluorophenyl)-4-oxo-1,4-dihydropyridazin-3-yl]methyl}phenyl)carbamate;
tetrahydro-2H-pyran-4-ylmethyl(3-{[1-(3,5-difluorophenyl)-4-oxo-1,4-dihydropyridazin-3-yl]methyl}-phenyl)carbamate;
rac-tetrahydro-2H-pyran-3-ylmethyl(3-{[1-(3,5-difluorophenyl)-4-oxo-1,4-dihydropyridazin-3-yl]methyl}phenyl)carbamate;
2-(2-methoxyethoxy)ethyl(3-{[1-(3,5-difluorophenyl)-4-oxo-1,4-dihydropyridazin-3-yl]methyl}phenyl)carbamate;
(2,2,6,6-tetramethyltetrahydro-2H-pyran-4-yl)methyl(3-{[1-(1-methyl-1H-pyrazol-4-yl)-4-oxo-1,4-dihydropyridazin-3-yl]methyl}phenyl)carbamate;
(2S)-2-[(2R or S)-2-methyl-5-oxopyrrolidin-1-yl]propyl (3-{[1-(1-methyl-1H-pyrazol-4-yl)-4-oxo-1,4-dihydropyridazin-3-yl]methyl}phenyl)carbamate;
rac-(2,2-dimethyltetrahydro-2H-pyran-4-yl)methyl(3-{[1-(1-methyl-1H-pyrazol-4-yl)-4-oxo-1,4-dihydropyridazin-3-yl]methyl}phenyl)carbamate;
rac-2-(4-hydroxy-2,2-dimethyltetrahydro-2H-pyran-4-yl)ethyl(3-{[1-(1-methyl-1H-pyrazol-4-yl)-4-oxo-1,4-dihydropyridazin-3-yl]methyl}phenyl)carbamate;
(4-fluorotetrahydro-2H-pyran-4-yl)methyl(3-{[1-(1-methyl-1H-pyrazol-4-yl)-4-oxo-1,4-dihydropyridazin-3-yl]methyl}phenyl)carbamate;
3-amino-2,2-difluoropropyl (3-{[1-(1-methyl-1H-pyrazol-4-yl)-4-oxo-1,4-dihydropyridazin-3-yl]methyl}phenyl)carbamate;
(2R)-pyrrolidin-2-ylmethyl(3-{[1-(1-methyl-1H-pyrazol-4-yl)-4-oxo-1,4-dihydropyridazin-3-yl]methyl}phenyl)carbamate;
(2S)-pyrrolidin-2-ylmethyl(3-{[1-(1-methyl-1H-pyrazol-4-yl)-4-oxo-1,4-dihydropyridazin-3-yl]methyl}phenyl)carbamate;
piperidin-4-ylmethyl(3-{[1-(1-methyl-1H-pyrazol-4-yl)-4-oxo-1,4-dihydropyridazin-3-yl]methyl}phenyl)carbamate;
piperidin-4-yl (3-{[1-(1-methyl-1H-pyrazol-4-yl)-4-oxo-1,4-dihydropyridazin-3-yl]methyl}phenyl)carbamate;
rac-2-amino-3,3,3-trifluoropropyl (3-{[1-(1-methyl-1H-pyrazol-4-yl)-4-oxo-1,4-dihydropyridazin-3-yl]methyl}phenyl)carbamate;
(4-fluoropiperidin-4-yl)methyl(3-{[1-(1-methyl-1H-pyrazol-4-yl)-4-oxo-1,4-dihydropyridazin-3-yl]methyl}phenyl)carbamate;
rac-3-amino-2-fluoropropyl (3-{[1-(1-methyl-1H-pyrazol-4-yl)-4-oxo-1,4-dihydropyridazin-3-yl]methyl}phenyl)carbamate;
2-(methylamino)ethyl(3-{[4-oxo-1-(3,4,5-trifluorophenyl)-1,4-dihydropyridazin-3-yl]methyl}phenyl)carbamate;
3-piperazin-1-ylpropyl (3-{[4-oxo-1-(3,4,5-trifluorophenyl)-1,4-dihydropyridazin-3-yl]methyl}phenyl)carbamate;
2-piperidin-4-ylethyl(3-{[1-(1-methyl-1H-pyrazol-4-yl)-4-oxo-1,4-dihydropyridazin-3-yl]methyl}phenyl)carbamate;
2-piperazin-1-ylethyl(3-{[1-(1-methyl-1H-pyrazol-4-yl)-4-oxo-1,4-dihydropyridazin-3-yl]methyl}phenyl)carbamate;
3-piperazin-1-ylpropyl (3-{[1-(1-methyl-1H-pyrazol-4-yl)-4-oxo-1,4-dihydropyridazin-3-yl]methyl}phenyl)carbamate;
azetidin-3-ylmethyl(3-{[1-(3-cyanophenyl)-4-oxo-1,4-dihydropyridazin-3-yl]methyl}phenyl)carbamate;
2-piperazin-1-ylethyl(3-{[1-(3,5-difluorophenyl)-4-oxo-1,4-dihydropyridazin-3-yl]methyl}phenyl)carbamate;
({[(3-{[1-(1-methyl-1H-pyrazol-4-yl)-4-oxo-1,4-dihydropyridazin-3-yl]methyl}phenyl)amino]carbonyl}oxy)acetic acid;
2-hydroxyethyl(3-{[1-(1-methyl-1H-pyrazol-4-yl)-4-oxo-1,4-dihydropyridazin-3-yl]methyl}phenyl)carbamate;
N-{3-[(4-Oxo-1-phenyl-1,4-dihydropyridazin-3-yl)methyl]phenyl}acetamide;
N-(3-{[1-(1-methyl-1H-pyrazol-4-yl)-4-oxo-1,4-dihydropyridazin-3-yl]methyl}phenyl)-2-phenylacetamide;
N-(3-{[1-(1-methyl-1H-pyrazol-4-yl)-4-oxo-1,4-dihydropyridazin-3-yl]methyl}phenyl)-3-phenylpropanamide;
N-(3-{[1-(1-methyl-1H-pyrazol-4-yl)-4-oxo-1,4-dihydropyridazin-3-yl]methyl}phenyl)butanamide;
N-(3-{[1-(1-methyl-1H-pyrazol-4-yl)-4-oxo-1,4-dihydropyridazin-3-yl]methyl}phenyl)propanamide;
1-(1-methyl-1H-pyrazol-4-yl)-3-[3-(2-oxopyrrolidin-1-yl)benzyl]pyridazin-4(1H)-one;
1-(1-methyl-1H-pyrazol-4-yl)-3-[3-(2-oxo-1,3-oxazolidin-3-yl)benzyl]pyridazin-4(1H)-one;
rac-2-fluoro-3-morpholin-4-ylpropyl (3-{[1-(1-methyl-1H-pyrazol-4-yl)-4-oxo-1,4-dihydropyridazin-3-yl]methyl}phenyl)carbamate;
rac-ethyl(3-{fluoro[1-(1-methyl-1H-pyrazol-4-yl)-4-oxo-1,4-dihydropyridazin-3-yl]methyl}phenyl)carbamate;
rac-ethyl{3-[fluoro(4-oxo-1-phenyl-1,4-dihydropyridazin-3-yl)methyl]phenyl}carbamate;
rac-ethyl{3-[[1-(4-chlorophenyl)-4-oxo-1,4-dihydropyridazin-3-yl](fluoro)methyl]phenyl}carbamate;
ethyl {3-[(4-oxo-1-phenyl-1,4-dihydropyridazin-3-yl)methyl]phenyl}carbamate;
ethyl[3-({1-[1-(2-hydroxyethyl)-1H-pyrazol-4-yl]-4-oxo-1,4-dihydropyridazin-3-yl}methyl)phenyl]carbamate;
propyl[3-({1-[1-(2-hydroxyethyl)-1H-pyrazol-4-yl]-4-oxo-1,4-dihydropyridazin-3-yl}methyl)phenyl]carbamate;
2-methylpropyl[3-({1-[1-(2-hydroxyethyl)-1H-pyrazol-4-yl]-4-oxo-1,4-dihydropyridazin-3-yl}methyl)phenyl]carbamate;
ethyl(3-{[1-(3-hydroxyphenyl)-4-oxo-1,4-dihydropyridazin-3-yl]methyl}phenyl)carbamate
ethyl[3-({1-[1-(2-aminoethyl)-1H-pyrazol-4-yl]-4-oxo-1,4-dihydropyridazin-3-yl}methyl)phenyl]carbamate;
2-methylpropyl[3-({1-[1-(2-aminoethyl)-1H-pyrazol-4-yl]-4-oxo-1,4-dihydropyridazin-3-yl}methyl)phenyl]carbamate;
ethyl(3-{[1-(3-methoxyphenyl)-4-oxo-1,4-dihydropyridazin-3-yl]methyl}phenyl)carbamate;
ethyl 3-((1-(3-ethoxyphenyl)-4-oxo-1,4-dihydropyridazin-3-yl)methyl)phenylcarbamate;
rac-ethyl{3-[1-(4-oxo-1-phenyl-1,4-dihydropyridazin-3-yl)ethyl]phenyl}carbamate;
2-methoxyethyl(3-{((1S or R)-1-[1-(1-methyl-1H-pyrazol-4-yl)-4-oxo-1,4-dihydropyridazin-3-yl]ethyl}phenyl)carbamate;
2-methoxyethyl(3-{(1R or S)-1-[1-(1-methyl-1H-pyrazol-4-yl)-4-oxo-1,4-dihydropyridazin-3-yl]ethyl}phenyl)carbamate;
rac-3-[[3-(5-ethoxypyrimidin-2-yl)phenyl](hydroxy)methyl]-1-(1-methyl-1H-pyrazol-4-yl)pyridazin-4(1H)-one;
3-[(S or R)-[3-(5-ethoxypyrimidin-2-yl)phenyl](fluoro)methyl]-1-(1-methyl-1H-pyrazol-4-yl)pyridazin-4(1H)-one;
3-[(R or S)-[3-(5-ethoxypyrimidin-2-yl)phenyl](fluoro)methyl]-1-(1-methyl-1H-pyrazol-4-yl)pyridazin-4(1H)-one;
rac-3-[[3-(5-ethoxypyrimidin-2-yl)phenyl](fluoro)methyl]-1-(1-methyl-1H-pyrazol-4-yl)pyridazin-4(1H)-one-d3;
rac-3-[[3-(5-ethoxypyrimidin-2-yl)phenyl](fluoro)methyl]-1-(1-methyl-1H-pyrazol-4-yl)pyridazin-4(1H)-one-d8;
rac-3-[[3-(5-ethoxypyrimidin-2-yl)phenyl](methoxy)methyl]-1-(1-methyl-1H-pyrazol-4-yl)pyridazin-4(1H)-one;
3-[3-(5-ethoxypyrimidin-2-yl)benzyl]-1-[1-(oxetan-3-yl)-1H-pyrazol-4-yl]pyridazin-4(1H)-one;
tert-butyl 3-(4-{3-[3-(5-ethoxypyrimidin-2-yl)benzyl]-4-oxopyridazin-1(4H)-yl}-1H-pyrazol-1-yl)azetidine-1-carboxylate;
2-(4-{3-[3-(5-ethoxypyrimidin-2-yl)benzyl]-4-oxopyridazin-1(4H)-yl}-1H-pyrazol-1-yl)-N,N-dimethylacetamide;
1-(1-azetidin-3-yl-1H-pyrazol-4-yl)-3-[3-(5-ethoxypyrimidin-2-yl)benzyl]pyridazin-4(1H)-one;
3-[3-(5-ethoxypyrimidin-2-yl)benzyl]-1-(1-piperidin-4-yl-1H-pyrazol-4-yl)pyridazin-4(1H)-one;
rac-4-[3-(5-ethoxypyrimidin-2-yl)phenyl]-4-[4-oxo-1-(1H-pyrazol-4-yl)-1,4-dihydropyridazin-3-yl]butanenitrile;
3-[3-(5-ethoxypyrimidin-2-yl)benzyl]-1-[1-(2-hydroxy-2-methylpropyl)-1H-pyrazol-4-yl]pyridazin-4(1H)-one;
2-methylpropyl {3-[(1-{1-[3-(dimethylamino)propyl]-1H-pyrazol-4-yl}-4-oxo-1,4-dihydropyridazin-3-yl)methyl]phenyl}carbamate;
3-[3-(5-ethoxypyrimidin-2-yl)benzyl]-1-[1-(1-methylazetidin-3-yl)-1H-pyrazol-4-yl]pyridazin-4(1H)-one;
1-phenyl-3-[3-(pyrimidin-2-yl)benzyl]pyridazin-4(1H)-one;
3-[3-(1-methyl-1H-1,2,4-triazol-3-yl)benzyl]-1-phenylpyridazin-4(1H)-one;
1-phenyl-3-(3-pyridin-2-ylbenzyl)pyridazin-4(1H)-one;
3-[3-(5-methyl-1H-imidazol-2-yl)benzyl]-1-(1-methyl-1H-pyrazol-4-yl)pyridazin-4(1H)-one;
3-[3-(1-methyl-1H-imidazol-4-yl)benzyl]-1-(1-methyl-1H-pyrazol-4-yl)pyridazin-4(1H)-one;
ethyl 2-(3-{[1-(1-methyl-1H-pyrazol-4-yl)-4-oxo-1,4-dihydropyridazin-3-yl]methyl}phenyl)-1,3-oxazole-4-carboxylate;
ethyl 2-(3-{[1-(1-methyl-1H-pyrazol-4-yl)-4-oxo-1,4-dihydropyridazin-3-yl]methyl}phenyl)-1,3-oxazole-5-carboxylate;
3-{3-[5-(hydroxymethyl)-1,3-thiazol-2-yl]benzyl}-1-(1-methyl-1H-pyrazol-4-yl)pyridazin-4(1H)-one;
3-[3-(5-ethoxypyridin-2-yl)benzyl]-1-(1-methyl-1H-pyrazol-4-yl)pyridazin-4(1H)-one;
rac-3-{1-[3-(4-ethoxypyrimidin-2-yl)phenyl]ethyl}-1-(1-methyl-1H-pyrazol-4-yl)pyridazin-4(1H)-one;
rac-3-(1-{3-[4-(2-methoxyethoxy)pyrimidin-2-yl]phenyl}ethyl)-1-(1-methyl-1H-pyrazol-4-yl)pyridazin-4(1H)-one;
3-{(1S or R)-1-[3-(1-ethyl-1H-1,2,4-triazol-3-yl)phenyl]ethyl}-1-(1-methyl-1H-pyrazol-4-yl)pyridazin-4(1H)-one;
3-{((1R or S)-1-[3-(1-ethyl-1H-1,2,4-triazol-3-yl)phenyl]ethyl}-1-(1-methyl-1H-pyrazol-4-yl)pyridazin-4(1H)-one;
1-(1-methyl-1H-pyrazol-4-yl)-3-{(S or R)-1-[3-(1-methyl-1H-1,2,4-triazol-3-yl)phenyl]ethyl}pyridazin-4(1H)-one;
1-(1-methyl-1H-pyrazol-4-yl)-3-{(1R or S)-1-[3-(1-methyl-1H-1,2,4-triazol-3-yl)phenyl]ethyl}pyridazin-4(1H)-one;
1-(1-methyl-1H-pyrazol-4-yl)-3-{(1S or R)-1-[3-(1-propyl-1H-1,2,4-triazol-3-yl)phenyl]ethyl}pyridazin-4(1H)-one;
1-(1-methyl-1H-pyrazol-4-yl)-3-{(R or S)-1-[3-(1-propyl-1H-1,2,4-triazol-3-yl)phenyl]ethyl}pyridazin-4(1H)-one;
1-(1-ethyl-1H-pyrazol-4-yl)-3-{(1S or R)-1-[3-(1-ethyl-1H-1,2,4-triazol-3-yl)phenyl]ethyl}pyridazin-4(1H)-one;
1-(1-ethyl-1H-pyrazol-4-yl)-3-{(1R or S)-1-[3-(1-ethyl-1H-1,2,4-triazol-3-yl)phenyl]ethyl}pyridazin-4(1H)-one;
1-(1-ethyl-1H-pyrazol-4-yl)-3-{(1S or R)-1-[3-(1-propyl-1H-1,2,4-triazol-3-yl)phenyl]ethyl}pyridazin-4(1H)-one;
1-(1-ethyl-1H-pyrazol-4-yl)-3-{(1R or S)-1-[3-(1-propyl-1H-1,2,4-triazol-3-yl)phenyl]ethyl}pyridazin-4(1H)-one;
3-[(1R or S)-1-{3-[4-(difluoromethyl)pyrimidin-2-yl]phenyl}ethyl]-1-(1-methyl-1H-pyrazol-4-yl)pyridazin-4(1H)-one;
1-(1-methyl-1H-pyrazol-4-yl)-3-{(1R or S)-1-[3-(4-methylpyrimidin-2-yl)phenyl]ethyl}pyridazin-4(1H)-one;
3-{(1R or S)-1-[3-(4-cyclopropyl-5-fluoropyrimidin-2-yl)phenyl]ethyl}-1-(1-methyl-1H-pyrazol-4-yl)pyridazin-4(1H)-one;
3-{(1R or S)-1-[3-(5-fluoro-4-methylpyrimidin-2-yl)phenyl]ethyl}-1-(1-methyl-1H-pyrazol-4-yl)pyridazin-4(1H)-one;
3-[(1R or S)-1-{3-[4-(2-hydroxypropan-2-yl)pyrimidin-2-yl]phenyl}ethyl]-1-(1-methyl-1H-pyrazol-4-yl)pyridazin-4(1H)-one;
3-[(1R or S)-1-(3-({4-[(2-methoxyethyl)amino]pyrimidin-2-yl}phenyl)ethyl]-1-(1-methyl-1H-pyrazol-4-yl)pyridazin-4(1H)-one;
2-methylpropyl {3-[(1-{1-[4-(dimethylamino)-4-oxobutyl]-1H-pyrazol-4-yl}-4-oxo-1,4-dihydropyridazin-3-yl)methyl]phenyl}carbamate;
2-methylpropyl {3-[(1-{1-[4-(methylamino)-4-oxobutyl]-1H-pyrazol-4-yl}-4-oxo-1,4-dihydropyridazin-3-yl)methyl]phenyl}carbamate;
2-methylpropyl[3-({1-[1-(4-morpholin-4-yl-4-oxobutyl)-1H-pyrazol-4-yl]-4-oxo-1,4-dihydropyridazin-3-yl}methyl)phenyl]carbamate;
2-methylpropyl. [3-({4-oxo-1-[1-(4-oxo-4-piperidin-1-ylbutyl)-1H-pyrazol-4-yl]-1,4-dihydropyridazin-3-yl}methyl)phenyl]carbamate;
2-methylpropyl[3-({4-oxo-1-[1-(4-oxo-4-pyrrolidin-1-ylbutyl)-1H-pyrazol-4-yl]-1,4-dihydropyridazin-3-yl}methyl)phenyl]carbamate;
2-methylpropyl {3-[(1-{1-[4-(oxetan-3-ylamino)-4-oxobutyl]-1H-pyrazol-4-yl}-4-oxo-1,4-dihydropyridazin-3-yl)methyl]phenyl}carbamate;
2-methylpropyl {3-[(1-{1-[3-(methylamino)-3-oxopropyl]-1H-pyrazol-4-yl}-4-oxo-1,4-dihydropyridazin-3-yl)methyl]phenyl}carbamate;
2-methylpropyl[3-({1-[1-(3-morpholin-4-yl-3-oxopropyl)-1H-pyrazol-4-yl]-4-oxo-1,4-dihydropyridazin-3-yl}methyl)phenyl]carbamate;
2-methylpropyl {3-[(1-{1-[3-(oxetan-3-ylamino)-3-oxopropyl]-1H-pyrazol-4-yl}-4-oxo-1,4-dihydropyridazin-3)-yl)methyl]phenyl}carbamate;
2-methylpropyl (3-{[1-(1-{4-[(2-hydroxy-2-methylpropyl)amino]-4-oxobutyl}-1H-pyrazol-4-yl)-4-oxo-1,4-dihydropyridazin-3-yl]methyl}phenyl)carbamate;
rac-2-methylpropyl (3-{[1-(1-{4-[(2-hydroxypropyl)amino]-4-oxobutyl}-1H-pyrazol-4-yl)-4-oxo-1,4-dihydropyridazin-3-yl]methyl}phenyl)carbamate;
1-(1-methyl-1H-pyrazol-4-yl)-3-{3-[1-(oxetan-3-yl)-1H-1,2,4-triazol-3-yl]benzyl}pyridazin-4(1H)-one;
3-[3-(1-ethyl-1H-1,2,4-triazol-3-yl)benzyl]-1-(1-methyl-1H-pyrazol-4-yl)pyridazin-4(1H)-one;
3-[3-(3-{[1-(1-methyl-1H-pyrazol-4-yl)-4-oxo-1,4-dihydropyridazin-3-yl]methyl}phenyl)-1H-1,2,4-triazol-1-yl]propanenitrile;
N,N-dimethyl-3-[3-(3-({[1-(1-methyl-1H-pyrazol-4-yl)-4-oxo-1,4-dihydropyridazin-3-yl]methyl}phenyl)-1-1H-1,2,4-triazol-1-yl]propanamide;
rac-1-(1-methyl-1H-pyrazol-4-yl)-3-(3-{1-[2-(tetrahydrofuran-2-yl)ethyl]-1H-1,2,4-triazol-3-yl}benzyl)pyridazin-4(1H)-one;
3-{3-[1-(2,2-difluoro-3-morpholin-4-ylpropyl)-1H-1,2,4-triazol-3-yl]benzyl}-1-(1-methyl-1H-pyrazol-4-yl)pyridazin-4(1H)-one;
3-(3-{1-[(3-methyloxetan-3-yl)methyl]-1H-1,2,4-triazol-3-yl}benzyl)-1-(1-methyl-1H-pyrazol-4-yl)pyridazin-4(1H)-one;
1-(1-methyl-1H-pyrazol-4-yl)-3-(3-{1-[2-(methylsulfonyl)ethyl]-1H-1,2,4-triazol-3-yl}benzyl)pyridazin-4(1H)-one;
1-(1-methyl-1H-pyrazol-4-yl)-3-{3-[1-(2-phenylethyl)-1H-1,2,4-triazol-3-yl]benzyl}pyridazin-4(1H)-one;
3-{3-[1-(2-ethoxyethyl)-1H-1,2,4-triazol-3-yl]benzyl}-1-(1-methyl-1H-pyrazol-4-yl)pyridazin-4(1-1)-one;
rac-1-(1-methyl-1H-pyrazol-4-yl)-3-(3-({1-[2-(tetrahydrofuran-3-yl)ethyl]-1H-1,2,4-triazol-3-yl}benzyl)pyridazin-4(1H)-one;
1-(1-methyl-1H-pyrazol-4-yl)-3-(3-{1-[2-(2-oxopyrrolidin-1-yl)ethyl]-1H-1,2,4-triazol-3-yl}benzyl)pyridazin-4(1H)-one;
3-{3-[1-(2-methoxy-2-methylpropyl)-1H-1,2,4-triazol-3-yl]benzyl}-1-(1-methyl-1H-pyrazol-4-yl)pyridazin-4(1H)-one;
3-(3-{1-[3-(5,5-dimethyl-1,3-dioxan-2-yl)propyl]-1H-1,2,4-triazol-3-yl}benzyl)-1-(1-methyl-1H-pyrazol-4-yl)pyridazin-4(1H)-one;
rac-1-(1-methyl-1H-pyrazol-4-yl)-3-(3-{1-[3-(tetrahydrofuran-2-yl)propyl]-1H-1,2,4-triazol-3-yl}benzyl)pyridazin-4(1H)-one;
rac-4-{3-[3-(5-ethoxypyrimidin-2-yl)benzyl]-4-oxopyridazin-1(4H)-yl}-2-fluoro-N-(2-hydroxyethyl)benzamide;
4-{3-[3-(5-ethoxypyrimidin-2-yl)benzyl]-4-oxopyridazin-1(4H)-yl}-2-fluoro-N-methylbenzamide;
4-{3-[3-(5-ethoxypyrimidin-2-yl)benzyl]-4-oxopyridazin-1(4H)-yl}-2-fluoro-N-(2-hydroxy-2-methylpropyl)benzamide;
3-(3-(5-ethoxypyrimidin-2-yl)benzyl)-1-(3-fluorophenyl)pyridazin-4(1H)-one;
3-{3-[3-(5-ethoxypyrimidin-2-yl)benzyl]-4-oxopyridazin-1(4H)-yl}-N,N-dimethylbenzamide;
2-methylpropyl[3-({4-oxo-1-[4-(pyridin-3-yl)phenyl]-1,4-dihydropyridazin-3-yl}methyl)phenyl]carbamate;
2-methylpropyl (3-{[4-oxo-1-(4-pyridin-4-ylphenyl)-1,4-dihydropyridazin-3-yl]methyl}phenyl)carbamate;
2-methylpropyl[3-({1-[3-(1-methyl-1H-pyrazol-4-yl)phenyl]-4-oxo-1,4-dihydropyridazin-3-yl}methyl)phenyl]carbamate;
2-methylpropyl (3-{[4-oxo-1-(3-pyridin-4-ylphenyl)-1,4-dihydropyridazin-3-yl]methyl}phenyl)carbamate;
2-methylpropyl[3-({4-oxo-1-[4-(1H-pyrazol-4-yl)phenyl-]-1,4-dihydropyridazin-3-yl}methyl)phenyl]carbamate;
2-methylpropyl[3-({1-[4-(1-methyl-1H-pyrazol-4-yl)phenyl]-4-oxo-1,4-dihydropyridazin-3-yl}methyl)phenyl]carbamate;
5-{3-[3-(5-methoxypyrimidin-2-yl)benzyl]-4-oxopyridazin-1(4H)-yl}pyridine-3-carbonitrile;
3-{3-[3-(1-ethyl-1H-1,2,4-triazol-3-yl)benzyl]-4-oxopyridazin-1(4H)-yl}benzamide;
3-{4-oxo-3-[3-(1-propyl-1H-1,2,4-triazol-3-yl)benzyl]pyridazin-1(4H)-yl}benzamide;
3-[3-[3-(5-ethoxypyrimidin-2-yl)benzyl]-4-oxopyridazin-1(4H)-yl]benzamide;
2-methoxyethyl[3-({1-[3-(aminocarbonyl)-5-fluorophenyl]-4-oxo-1,4-dihydropyridazin-3-yl}methyl)phenyl]carbamate;
rac-3-[3-{-[3-(5-ethoxypyrimidin-2-yl)phenyl]ethyl}-4-oxopyridazin-1(4H)-yl]benzamide;
3-[3-(5-ethyl-1,2,4-oxadiazole-3-yl)benzyl]-1-(1-methyl-1H-pyrazol-4-yl)pyridazin-4(1H)-one;
1-(1-methyl-1H-pyrazol-4-yl)-3-[3-(5-propyl-1,2,4-oxadiazol-3-yl)benzyl]pyridazin-4(1H)-one;
3-[3-(5-butyl-1,2,4-oxadiazol-3-yl)benzyl]-1-(1-methyl-1H-pyrazol-4-yl)pyridazin-4(1H)-one;
3-{3-[5-(2-methylpropyl)-1,2,4-oxadiazol-3-yl]benzyl}-1-(1-methyl-1H-pyrazol-4-yl)pyridazin-4(1H)-one;
1-(1-methyl-1H-pyrazol-4-yl)-3-{3-[5-(morpholin-4-ylmethyl)-1,2,4-oxadiazol-3-yl]benzyl}pyridazin-4(1H)-one;
3-{3-[5-(2-methoxyethyl)-1,2,4-oxadiazol-3-yl]benzyl}-1-(1-methyl-1H-pyrazol-4-yl)pyridazin-4(1H)-one;
1-(1-methyl-1H-pyrazol-4-yl)-3-{3-[5-(oxetan-3-ylmethyl)-1,2,4-oxadiazol-3-yl]benzyl}pyridazin-4(1H)-one;
1-(1-methyl-1H-pyrazol-4-yl)-3-{3-[5-(tetrahydro-2H-pyran-4-ylmethyl)-1,2,4-oxadiazol-3-yl]benzyl}pyridazin-4(1H)-one;
3-(3-{5-[(trans-4-hydro oxyl)oxy]pyrimidin-2-}benzyl)-1-(1-methyl-1H-pyrazol-4-yl)pyridazin-4(1H)-one;
3-(3-{5-[(cis-4-hydroxycyclohexyl)oxy]pyrimidin-2-yl}benzyl)-1-(1-methyl-1H-pyrazol-4-yl)pyridazin-4(1H)-one;
rac-3-{3-[5-(trans-4-fluoro-3-hydroxypiperidin-4-yl)pyrimidin-2-yl]benzyl}-1-(1-methyl-1H-pyrazol-4-yl)pyridazin-4(1H)-one;
3-{3-[5-(2-methoxyethoxy)pyrimidin-2-yl]benzyl}-5-methyl-1-(1-methyl-1H-pyrazol-4-yl)pyridazin-4(1H)-one;
5-fluoro-1-(5-fluoro-1-methyl-1H-pyrazol-4-yl)-3-{3-[5-(2-methoxyethoxy)pyrimidin-2-yl]benzyl}pyridazin-4(1H)-one;
3-[3-(5-ethoxypyrimidin-2-yl)benzyl]-5-fluoro-1-(5-fluoro-1-methyl-1H-pyrazol-4-yl)pyridazin-4(1H)-one;
rac-3-[fluoro(quinolin-6-yl)methyl]-1-(1-methyl-1H-pyrazol-4-yl)pyridazin-4(1H)-one;
1-(4-chlorophenyl)-3-(4-hydroxybenzyl)pyridazin-4(1H)-one;
3-[3-(5-ethoxypyrimidin-2-yl)phenoxy]-1-(1-methyl-1H-pyrazol-4-yl)pyridazin-4(1H)-one;
3-{[3-(5-ethoxypyrimidin-2-yl)phenyl]sulfanyl}-1-(1-methyl-1H-pyrazol-4-yl)pyridazin-4(1H)-one;
   or a pharmaceutically acceptable salt thereof.

In a class of the invention, specific compounds include, but are not limited to:
3-[3-(5-ethoxypyrimidin-2-yl)benzyl]-1-(1-methyl-1H-pyrazol-4-yl)pyridazin-4(1H)-one;
3-{3-[5-(2-methoxyethoxy)pyrimidin-2-yl]benzyl}-1-(1-methyl-1H-pyrazol-4-yl)pyridazin-4(1H)-one;
ethyl(3-{[1-(1-methyl-1H-pyrazol-4-yl)-4-oxo-1,4-dihydropyridazin-3-yl]methyl}phenyl)carbamate;
3-{3-[5-(2-hydroxy-2-methylpropoxy)pyrimidin-2-yl]benzyl}-1-(1-methyl-1H-pyrazol-4-yl)pyridazin-4(1H)-one;
tetrahydrofuran-2-ylmethyl(3-{[1-(1-methyl-1H-pyrazol-4-yl)-4-oxo-1,4-dihydropyridazin-3-yl]methyl}phenyl)carbamate;
tetrahydro-2H-pyran-3-ylmethyl(3-{[1-(1-methyl-1H-pyrazol-4-yl)-4-oxo-1,4-dihydropyridazin-3-yl]methyl}phenyl)carbamate;
3-methoxypropyl (3-{[1-(1-methyl-1H-pyrazol-4-yl)-4-oxo-1,4-dihydropyridazin-3-yl]methyl}phenyl)carbamate;
2-methoxyethyl(3-{[1-(1-methyl-1H-pyrazol-4-yl)-4-oxo-1,4-dihydropyridazin-3-yl]methyl}phenyl)carbamate;
propyl (3-{[1-(1-methyl-1H-pyrazol-4-yl)-4-oxo-1,4-dihydropyridazin-3-yl]methyl}phenyl)carbamate;
2-methoxyethyl(3-{[1-(3,4-difluorophenyl)-4-oxo-1,4-dihydropyridazin-3-yl]methyl}phenyl)carbamate;
tetrahydrofuran-3-ylmethyl(3-{[1-(1-methyl-1H-pyrazol-4-yl)-4-oxo-1,4-dihydropyridazin-3-yl]methyl}phenyl)carbamate;
1-(1-methyl-1H-pyrazol-4-yl)-3-{3-[5-(tetrahydrofuran-2-ylmethoxy)pyrimidin-2-yl]benzyl}pyridazin-4(1H)-one;
2-methylpropyl[3-({1-[1-(2-hydroxyethyl)-1H-pyrazol-4-yl]-4-oxo-1,4-dihydropyridazin-3-yl}methyl)phenyl]carbamate;
3-fluoro-5-[3-{3-[5-(2-hydroxy-2-methylpropoxy)pyrimidin-2-yl]benzyl}-4-oxopyridazin-1(4H)-yl]benzonitrile;
3-{(1R or S)-1-[3-(5-methoxypyrimidin-2-yl)phenyl]ethyl}-1-(1-methyl-1H-pyrazol-4-yl)pyridazin-4(1H)-one;
3-((1R or S)-1-{3-[5-(2-hydroxy-2-methylpropoxy)pyrimidin-2-yl]phenyl}ethyl)-1-(1-methyl-1H-pyrazol-4-yl)pyridazin-4(1H)-one;
   or a pharmaceutically acceptable salt or stereoisomer thereof.

The compounds of the present invention may have asymmetric centers, chiral axes, and chiral planes (as described in: E. L. Eliel and S. H. Wilen, Stereochemistry of Carbon Compounds, John Wiley & Sons, New York, 1994, pages 1119-1190), and occur as racemates, racemic mixtures, and as individual diastereomers, with all possible isomers and mixtures thereof, including optical isomers, all such stereoisomers being included in the present invention.

In addition, the compounds disclosed herein may exist as tautomers and both tautomeric forms are intended to be encompassed by the scope of the invention, even though only one tautomeric structure is depicted. In the compounds of generic Formula I, the atoms may exhibit their-natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula I. For example, different isotopic forms of hydrogen (H) include protium (1H) and deuterium (2H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within generic Formula I can be prepared without undue experimentation by conventional techniques well known to those skilled in the art or by processes analogous to those described in the Schemes and Examples herein using appropriate isotopically-enriched reagents and/or intermediates.

When any variable (e.g., R¹⁰) occurs more than one-time in any constituent, its definition on each occurrence is independent at every other occurrence. Also, combinations of substituents and variables are permissible only if such combinations result in stable compounds. Lines drawn into the ring systems from substituents represent that the indicated bond may be attached to any of the substitutable ring atoms. If the ring system is polycyclic, it is intended that the bond be attached to any of the suitable carbon atoms on the proximal ring only.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results. The phrase “optionally substituted with one or more substituents” should be taken to be equivalent to the phrase “optionally substituted with at least one substituent” and in such cases another embodiment will have from zero to three substituents.

As used herein, “alkyl” is intended to include both branched and straight-chain saturated aliphatic hydrocarbon groups having the specified number of carbon atoms. For example, C₁-C₁₀, as in “C₁-C₁₀ alkyl” is defined to include groups having 1, 2, 3, 4, 5, 6, 7, 8, 9 or 10 carbons in a linear or branched arrangement. For example, “C₁-C₁₀ alkyl” specifically includes methyl, ethyl, n-propyl, i-propyl, n-butyl, t-butyl, i-butyl, pentyl, hexyl, heptyl, octyl, nonyl, decyl, and so on. The term “cycloalkyl” means a monocyclic saturated aliphatic hydrocarbon group having the specified number of carbon atoms. For example, “cycloalkyl” includes cyclopropyl, methyl-cyclopropyl, 2,2-dimethyl-cyclobutyl, 2-ethyl-cyclopentyl, cyclohexyl, and so on. In an embodiment of the invention the term “cycloalkyl” includes the groups described immediately above and further includes monocyclic unsaturated aliphatic hydrocarbon groups. For example, “cycloalkyl” as defined in this embodiment includes cyclopropyl, methyl-cyclopropyl, 2,2-dimethyl-cyclobutyl, 2-ethyl-cyclopentyl, cyclohexyl, cyclopentenyl, cyclobutenyl and so on.

The term “haloalkyl” means an alkyl radical as defined above, unless otherwise specified, that is substituted with one to five, preferably one to three halogen. Representative examples include, but are not limited to trifluoromethyl, dichloroethyl, and the like.

“Alkoxy” represents either a cyclic or non-cyclic alkyl group of indicated number of carbon atoms attached through an oxygen bridge. “Alkoxy” therefore encompasses the definitions of alkyl and cycloalkyl above.

In certain instances, substituents may be defined with a range of carbons that includes zero, such as (C₀-C₆)alkylene-aryl. If aryl is taken to be phenyl, this definition-would include phenyl itself as well as —CH₂Ph, —CH₂CH₂Ph, CH(CH₃)CH₂CH(CH₃)Ph, and so on.

As used herein, “aryl” is intended to mean any stable monocyclic or bicyclic carbon ring of up to 7 atoms in each ring, wherein at least one ring is aromatic. Examples of such aryl elements include phenyl, naphthyl, tetrahydronaphthyl, indanyl and biphenyl. In cases where the aryl substituent is bicyclic and one ring is non-aromatic, it is understood that attachment is via the aromatic ring.

The term “heteroaryl,” as used herein, represents a stable monocyclic or bicyclic ring of up to 7 atoms in each ring, wherein at least one ring is aromatic and contains from 1 to 4 heteroatoms selected from the group consisting of O, N and S. Heteroaryl groups within the scope of this definition include but are not limited to: acridinyl, carbazolyl, cinnolinyl, quinoxalinyl, pyrrazolyl, indolyl, benzotriazolyl, furanyl, thienyl, benzothienyl, benzofuranyl, benzimidazolonyl, benzoxazolonyl, quinolinyl, isoquinolinyl, dihydroisoindolonyl, imidazopyridinyl, isoindolonyl, indazolyl, oxazolyl, oxadiazolyl, isoxazolyl, indolyl, pyrazinyl, pyridazinyl, pyridinyl, pyrimidinyl, pyrrolyl, tetrahydroquinoline. As with the definition of heterocycle below, “heteroaryl” is also understood to include the N-oxide derivative of any nitrogen-containing heteroaryl. In cases where the heteroaryl substituent is bicyclic and one ring is non-aromatic or contains no heteroatoms, it is understood that attachment is via the aromatic ring or via the heteroatom containing ring, respectively.

The term “heterocycle” or “heterocyclyl” as used herein is intended to mean a 3- to 10-membered aromatic or nonaromatic heterocycle containing from 1 to 4 heteroatoms selected from the group consisting of O, N and S, and includes bicyclic groups. For the purposes of this invention, the term “heterocyclic” is also considered to be synonymous with the terms “heterocycle” and “heterocyclyl” and is understood as also having the definitions set forth herein. “Heterocyclyl” therefore includes the above mentioned heteroaryls, as well as dihydro and tetrathydro analogs thereof. Further examples of “heterocyclyl” include, but are not limited to the following: azetidinyl, benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxooxazolidinyl, oxazolyl, oxazoline, oxopiperazinyl, oxopyrrolidinyl, oxomorpholinyl, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrahydrofuranyl, tetrahydrothiopyranyl, tetrahydroisoquinolinyl, tetrazolyl, tetrazolopyridyl, thiadiazolyl, thiazolyl, thienyl, triazolyl, 1,4-dioxanyl, hexahydroazepinyl, piperazinyl, piperidinyl, pyridin-2-onyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, dioxidothiomorpholinyl, methylenedioxybenzoyl, tetrahydrofuranyl, and tetrahydrothienyl, and N-oxides thereof. Attachment of a heterocyclyl substituent can occur via a carbon atom or via a heteroatom.

As appreciated by those of skill in the art, “halo” or “halogen” as used herein is intended to include chloro, fluoro, bromo and iodo.

The alkyl, cycloalkyl, aryl, heteroaryl and heterocyclyl substituents may be substituted or unsubstituted, unless specifically defined otherwise. For example, a (C₁-C₆)alkyl may be substituted with one, two or three substituents selected from OH, oxo, halogen, alkoxy, dialkylamino, or heterocyclyl, such as morpholinyl, piperidinyl, and so on. In this case, if one substituent is oxo and the other is OH, the following are included in the definition: —C═O)CH₂CH(OH)CH₃, —(C═O)OH, —CH₂(OH)CH₂CH(O), and so on.

Included in the instant invention is the free form of compounds of the instant invention, as well as the pharmaceutically acceptable salts and stereoisomers thereof. The term “free form” refers to the amine compounds in non-salt form. The encompassed pharmaceutically acceptable salts not only include the salts exemplified for the specific compounds described herein, but also all the typical pharmaceutically acceptable salts of the free form of compounds of the instant invention. The free form of the specific salt compounds described may be isolated using techniques known in the art. For example, the free form may be regenerated by treating the salt with a suitable dilute aqueous base solution such as dilute aqueous NaOH, potassium carbonate, ammonia and sodium bicarbonate. The free forms may differ from their respective salt forms somewhat in certain physical properties, such as solubility in polar solvents, but the acid and base salts are otherwise pharmaceutically equivalent to their respective free forms for purposes of the invention.

The pharmaceutically acceptable salts of the instant compounds can be synthesized from the compounds of this invention which contain a basic or acidic moiety by conventional chemical methods. Generally, the salts of the basic compounds are prepared either by ion exchange chromatography or by reacting the free base with stoichiometric amounts or with an excess of the desired salt-forming inorganic or organic acid in a suitable solvent or various combinations of solvents. Similarly, the salts of the acidic compounds are formed by reactions with the appropriate inorganic or organic base.

Thus, pharmaceutically acceptable salts of the compounds of this invention include the conventional non-toxic salts of the compounds of this invention as formed by reacting a basic instant compound with an inorganic or organic acid. For example, conventional non-toxic salts include those derived from inorganic acids such as hydrochloric, hydrobromic, sulfuric, sulfamic, phosphoric, nitric and the like, as well as salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxy-benzoic, fumaric, toluenesulfonic, methanesulfonic, ethane disulfonic, oxalic, isethionic, trifluoroacetic and the like.

When the compound of the present invention is acidic, suitable “pharmaceutically acceptable salts” refers to salts prepared form pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc and the like. Particularly preferred are the ammonium, calcium, magnesium, potassium and sodium salts. Salts derived from pharmaceutically acceptable organic non-toxic bases include salts of primary, secondary and tertiary amines, substituted amines including naturally occurring substituted amines, cyclic amines and basic ion exchange resins, such as arginine, betaine caffeine, choline, N,N¹-dibenzylethylenediamine, diethylamin, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like. When the compound of the present invention is acidic, the term “free form” refers to the compound in its non-salt form, such that the acidic functionality is still protonated.

The preparation of the pharmaceutically-acceptable salts described above and other typical pharmaceutically acceptable salts is more fully described by Berg et al., “Pharmaceutical Salts,” J. Pharm. Sci., 1977:66:1-19.

It will also be noted that the compounds of the present invention may potentially be internal salts or zwitterions, since under physiological conditions a deprotonated acidic moiety in the compound, such as a carboxyl group, may be anionic, and this electronic charge might then be balanced off internally against the cationic charge of a protonated or alkylated basic moiety, such as a quaternary nitrogen atom. An isolated compound having internally balance charges, and thus not associated with a intermolecular counterion, may also be considered the “free form” of a compound.

### Utilities

The compounds of the invention are useful to bind to and/or modulate the activity of a tyrosine kinase, in particular, a receptor tyrosine kinase. In an embodiment, the receptor tyrosine kinase is a member of the MET subfamily. In a further embodiment, the MET is human MET, although the activity of receptor tyrosine kinases from other organisms may also be modulated by the compounds of the present invention. In this context, modulate means either increasing or decreasing kinase activity of MET. In an embodiment, the compounds of the instant invention inhibit the kinase activity of MET.

The compounds of the invention find use in a variety of applications. As will be appreciated by those skilled in the art, the kinase activity of MET may be modulated in a variety of ways; that is, one can affect the phosphorylation/activation of MET either by modulating the initial phosphorylation of the protein or by modulating the autophosphorylation of the other active sites of the protein. Alternatively, the kinase activity of MET may be modulated by affecting the binding of a substrate of MET phosphorylation.

The compounds of the invention are used to treat or prevent cellular proliferation diseases. Disease states which can be treated by the methods and compositions provided herein include, but are not limited to, cancer (further discussed below), autoimmune disease, arthritis, graft rejection, inflammatory bowel disease, proliferation induced after medical procedures, including, but not limited to, surgery, angioplasty, and the like. It is appreciated that in some cases the cells may not be in a hyper- or hypoproliferation state (abnormal state) and still require treatment. Thus, in one embodiment, the invention herein includes application to cells or individuals which are afflicted or may eventually become afflicted with any one of these disorders or states.

The compounds, compositions and methods provided herein are particularly deemed useful for the treatment and prevention of cancer including solid tumors such as skin, breast, brain, cervical carcinomas, testicular carcinomas, etc. In an embodiment, the instant compounds are useful for treating cancer. In particular, cancers that may be treated by the compounds, compositions and methods of the invention include, but are not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor [nephroblastoma], lymphoma, leukemia,), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoidosteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord (neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma. Thus, the term “cancerous cell” as provided herein, includes a cell afflicted by any one of the above-identified conditions. In an embodiment of the invention, cancers that may be treated by the compounds, compositions and methods of the invention include, in addition to the cancers listed above: Lung: bronchogenic carcinoma (non-small cell lung); Gastrointestinal: rectal, colorectal and colon; Genitourinary tract: kidney (papillary renal cell carcinoma); and Skin: head and neck squamous cell carcinoma.

In another embodiment, the compounds of the instant invention are useful for treating or preventing cancer selected from: head and neck squamous cell carcinomas, histiocytic lymphoma, lung adenocarcinoma, small cell lung cancer, non-small cell lung cancer, pancreatic cancer, papillary renal cell carcinoma, liver cancer, gastric cancer, colon cancer, multiple myeloma, glioblastomas and breast carcinoma. In yet another embodiment, the compounds of the instant invention are useful for treating or preventing cancer selected from: histiocytic lymphoma, lung adenocarcinoma, small cell lung cancer, pancreatic cancer, liver cancer, gastric cancer, colon cancer, multiple myeloma, glioblastomas and breast carcinoma. In still another embodiment, the compounds of the instant invention are useful for treating cancer selected from: histiocytic lymphoma, lung adenocarcinoma, small cell lung cancers, pancreatic cancer, liver cancer, gastric cancer, colon cancer, multiple myeloma, glioblastomas and breast carcinoma.

In another embodiment, the compounds of the instant invention are useful for the prevention or modulation of the metastases of cancer cells and cancer. In particular, the compounds of the instant invention are useful to prevent or modulate the metastases of ovarian cancer, childhood hepatocellular carcinoma, metastatic head and neck squamous cell carcinomas, gastric cancers, breast cancer, colorectal cancer, cervical cancer, lung cancer, nasopharyngeal cancer, pancreatic cancer, glioblastoma and sarcomas.

The compounds of this invention may be administered to mammals, preferably humans, either alone or in combination with pharmaceutically acceptable carriers, excipients or diluents, in a pharmaceutical composition, according to standard pharmaceutical practice. The compounds can be administered orally or parenterally, including the intravenous, intramuscular, intraperitoneal, subcutaneous, rectal and topical routes of administration.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, microcrystalline cellulose, sodium crosscarmellose, corn starch, or alginic acid; binding agents, for example starch, gelatin, polyvinyl-pyrrolidone or acacia, and lubricating agents, for example, magnesium stearate, stearic acid or talc. The tablets may be uncoated or they may be coated by known techniques to mask the unpleasant taste of the drug or delay disintegration and absorption in the gastrointestinal tract and thereby provide a sustained action over a longer period. For example, a water soluble taste masking material such as hydroxypropyl-methylcellulose or hydroxypropylcellulose, or a time delay material such as ethyl cellulose, cellulose acetate butyrate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty-acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

The pharmaceutical compositions of the invention may also be in the form of an oil-in-water emulsions. The oily phase may be a vegetable oil, for example olive oil or arachis oil, or a mineral oil, for example liquid paraffin or mixtures of these. Suitable emulsifying agents may be naturally occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening, flavoring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

The pharmaceutical compositions may be in the form of a sterile injectable aqueous solutions. Among the acceptable vehicles and solvents that may be employed are water, Ringer's solution and isotonic sodium chloride solution.

The sterile injectable preparation may also be a sterile injectable oil-in-water microemulsion where the active ingredient is dissolved in the oily phase. For example, the active ingredient may be first dissolved in a mixture of soybean oil and lecithin. The oil solution then introduced into a water and glycerol mixture and processed to form a microemulation.

The injectable solutions or microemulsions may be introduced into a patient's blood stream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump.

The pharmaceutical compositions may be in the form of a sterile injectable aqueous or oleagenous suspension for intramuscular and subcutaneous administration. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, for example as a solution in 1,3-butane diol. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds of Formula I may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing the compound of Formula I are employed. (For purposes of this application, topical application shall include mouth washes and gargles.)

The compounds for the present invention can be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will, of course, be continuous rather than intermittent throughout the dosage regimen. Compounds of the present invention may also be delivered as a suppository employing bases such as cocoa butter, glycerinated gelatin, hydrogenated vegetable oils, mixtures of polyethylene glycols of various molecular weights and fatty acid esters of polyethylene glycol.

The dosage regimen utilizing the compounds of the instant invention can be selected in accordance with a variety of factors including type, species, age, weight, sex and the type of cancer being treated; the severity (i.e., stage) of the cancer to be treated; the route of administration; the renal and hepatic function of the patient; and the particular compound or salt thereof employed. An ordinarily skilled physician or veterinarian can readily determine and prescribe the effective amount of the drug required to treat, for example, to prevent, inhibit (fully or partially) or arrest the progress of the disease.

In one exemplary-application, a suitable amount of compound is administered to a mammal undergoing treatment for cancer. Administration occurs in an amount between about 0.1 mg/kg of body weight to about 60 mg/kg of body weight per day, preferably of between 0.5 mg/kg of body weight to about 40 mg/kg of body weight per day.

In a further example, compounds of the instant invention can be administered in a total daily dose of up to 1000 mg. Compounds of the instant invention can be administered once daily (QD), or divided into multiple daily doses such as twice daily (BID), and three times daily (TID). Compounds of the instant invention can be administered at a total daily dosage of up to 1000 mg, e.g., 200 mg, 300 mg, 400 mg, 600 mg, 800 mg or 1000 mg, which can be administered in one daily dose or can be divided into multiple daily doses as described above.

In addition, the administration can be continuous, i.e., every day, or intermittently. The terms “intermittent” or “intermittently” as used herein means stopping and starting at either regular or irregular intervals. For example, intermittent administration of a compound of the instant invention may be administration one to six days per week or it may mean administration in cycles (e.g., daily administration for two to eight consecutive weeks, then a rest period with no administration for up to one week) or it may mean administration on alternate days.

In addition, the compounds of the instant invention may be administered according to any of the schedules described above, consecutively for a few weeks, followed by a rest period. For example, the compounds of the instant invention may be administered according to any one of the schedules described above from two to eight weeks, followed by a rest period of one week, or twice daily at a dose of 100-500 mg for three to five days a week. In another particular embodiment, the compounds of the instant invention may be administered three times daily for two consecutive weeks, followed by one week of rest.

The instant compounds are also useful in combination with known therapeutic agents and anti-cancer agents. For example, instant compounds are useful in combination with known anti-cancer agents. Combinations of the presently disclosed compounds with other anti-cancer or chemotherapeutic agents are within the scope of the invention. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Such anti-cancer agents include, but are not limited to, the following: estrogen receptor modulators, androgen receptor modulators, retinoid receptor modulators, cytotoxic/cytostatic agents, antiproliferative agents, prenyl-protein transferase inhibitors, HMG-CoA reductase inhibitors and other angiogenesis inhibitors, inhibitors of cell proliferation and survival signaling, apoptosis inducing agents and agents that interfere with cell cycle checkpoints. The instant compounds are particularly useful when co-administered with radiation therapy.

In an embodiment, the instant compounds are also useful in combination with known anti-cancer agents including the following: estrogen receptor modulators, androgen receptor-modulators, retinoid receptor modulators, cytotoxic agents, antiproliferative agents, prenyl-protein transferase inhibitors, HMG-CoA reductase inhibitors, HIV protease-inhibitors, reverse transcriptase inhibitors, and other angiogenesis inhibitors.

“Estrogen receptor modulators” refers to compounds that interfere with or inhibit the binding of estrogen to the receptor, regardless of mechanism. Examples of estrogen receptor modulators include, but are not limited to, tamoxifen, raloxifene, idoxifene, LY353381, LY117081, toremifene, fulvestrant, 4-[7-(2,2-dimethyl-1-oxopropoxy-4-methyl-2-[4-[2-(1-piperidinyl)ethoxy]phenyl]-2H-1-benzopyran-3-yl]-phenyl-2,2-dimethylpropanoate, 4,4′-dihydroxybenzophenone-2,4-dinitrophenyl-hydrazone, and SH646.

“Androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

“Retinoid receptor modulators” refers to compounds which interfere or inhibit the binding of retinoids to the receptor, regardless of mechanism. Examples of such retinoid receptor modulators include bexarotene, tretinoin, 13-cis-retinoic acid, 9-cis-retinoic acid, α-difluoromethylornithine, ILX23-7553, trans-N-(4′-hydroxyphenyl) retinamide, and N-4-carboxyphenyl retinamide.

“Cytotoxic/cytostatic agents” refer to compounds which cause cell death or inhibit cell proliferation primarily by interfering directly with the cell's functioning or inhibit or interfere with cell mytosis, including alkylating-agents, tumor necrosis factors, intercalators, hypoxia-activatable compounds, microtubule inhibitors/microtubule-stabilizing agents, inhibitors of mitotic kinesins, inhibitors of histone deacetylase, inhibitors of kinases involved in mitotic progression, antimetabolites; biological response modifiers; hormonal/anti-hormonal therapeutic agents, haematopoietic growth factors, monoclonal antibody targeted therapeutic agents, topoisomerase inhibitors, proteasome inhibitors and ubiquitin ligase inhibitors.

Examples of cytotoxic agents include, but are not limited to, sertenef, cachectin, ifosfamide, tasonermin, lonidamine, carboplatin, altretamine, prednimustine, dibromodulcitol, ranimustine, fotemustine, nedaplatin, oxaliplatin, temozolomide, heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-aminedichloro(2-methyl-pyridine)platinum, benzylguanine, glufosfamide, GPX100, (trans, trans, trans)-bis-mu-(hexane-1,6-diamine)-mu-[diamine-platinum(II)]bis[diamine(chloro)platinum (II)]tetrachloride, diarizidinyispermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deamino-3′-morpholino-13-deoxo-10-hydroxycaminomycin, annamycin, galarubicin, elinafide, MEN10755, and 4-demethoxy-3-deamino-3-aziridinyl-4-methylsulphonyl-daunorubicin (see WO 00/50032).

An example of a hypoxia activatable compound is tirapazamine.

Examples of proteasome inhibitors include but are not limited to lactacystin and bortezomib.

Examples of microtubule inhibitors/microtubule-stabilising agents include paclitaxel, vindesine sulfate, 3′,4′-didehydro-4′-deoxy-8′-norvincaleukoblastine, docetaxol, rhizoxin, dolastatin, mivobulin isethionate, auristatin, cemadotin, RPR109881, BMS184476, vinflunine, cryptophycin, 2,3,4,5,6-pentafluoro-N-(3-fluoro-4-methoxyphenyl)benzene sulfonamide, anhydrovinblastine, N,N-dimethyl-L-valyl-L-valyl-N-methyl-L-valyl-L-prolyl-L-proline-t-butylamide, TDX258, the epothilones (see for example U.S. Pat. Nos. 6,284,781 and 6,288,237) and BMS188797.

Some examples of topoisomerase inhibitors are topotecan, hycaptamine; irinotecan, rubitecan, 6-ethoxypropionyl-3′,4′-O-exo-benzylidene-chartreusin, 9-methoxy-N,N-dimethyl-5-nitropyrazolo[3,4,5-k1]acridine-2-(6H) propanamine, 1-amino-9-ethyl-5-fluoro-2,3-dihydro-9-hydroxy-4-methyl-1H, 12H-benzo[de]pyrano[3′,4′:b,7]-indolizino[1,2b]quinoline-10,13(9H,15H)dione, lurtotecan, 7-[2-(N-isopropylamino)ethyl]-(20S)camptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a,5aB,8aa,9b)-9-[2-[N-[2-(dimethylamino)ethyl]-N-methylamino]ethyl]-5-[4-hydro0xy-3,5-dimethoxyphenyl]-5,5a,6,8,8a,9-hexohydrofuro(3′,4′:6,7)naphtho(2,3-d)-1,3-dioxol-6-one, 2,3-(methylenedioxy)-5-methyl-7-hydroxy-8-methoxybenzo[c]-phenanthridinium, 6,9-bis[(2-aminoethyl)amino]benzo[g]isoquinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2(diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2,1-c]quinolin-7-one, and dimesna.

Examples of inhibitors of mitotic kinesins, and in particular the human mitotic kinesin KSP, are described in PCT Publications WO 01/30768, WO 01/98278, WO 03/050,064, WO 03/050,122, WO 03/049,527, WO 03/049,679, WO 03/049,678, WO04/039774, WO03/079973, WO03/099211, WO03/105855, WO03/106417, WO04/037171, WO04/058148, WO04/058700, WO04/126699, WO05/018638, WO05/019206, WO05/019205, WO05/018547, WO05/017190, US2005/0176776. In an embodiment inhibitors of mitotic kinesins include, but are not limited to inhibitors of KSP, inhibitors of MKLP1, inhibitors of CENP-E, inhibitors of MCAK, inhibitors of Kifl4, inhibitors of Mphosph1 and inhibitors of Rab6-KIFL.

Examples of “histone deacetylase inhibitors” include, but are not limited to, SAHA, TSA, oxamflatin, PXD101, MG98, valproic acid and scriptaid. Further reference to other histone deacetylase inhibitors may be found in the following manuscript; Miller, T. A. et al. J. Med. Chem. 46(24):5097-5116 (2003).

“Inhibitors of kinases involved in mitotic progression” include, but are not limited to, inhibitors of aurora kinase, inhibitors of Polo-like kinases (PLK) (in particular inhibitors of PLK-1), inhibitors of bub-1 and inhibitors of bub-R1.

“Antiproliferative agents” includes antisense RNA and DNA oligonucleotides such as G3139, ODN698, RVASKRAS, GEM231, and INX3001, and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxycytidine, N-[5-(2,3-dihydro-benzofuryl)sulfonyl]-N′-(3,4-dichlorophenyl)urea, N6-[4-deoxy-4-[N2-[2(E),4(E)-tetradecadienoyl]glycylamino]-L-glycero-B-L-manno-heptopyranosyl]adenine, aplidine, ecteinascidin, troxacitabine, 4-[2-amino-4-oxo-4,6,7,8-tetrahydro-3H-pyrimidino[5,4-b][1,4]thiazin-6-yl-(S)-ethyl]-2,5-thienoyl-L-glutamic acid, aminopterin, 5-fluorouracil, alanosine, 11-acetyl-8-(carbamoyloxymethyl)-4-formyl-6-methoxy-14-oxa-1,11-diazatetracyclo(7.4.1.0.0)-tetradeca-2,4,6-trien-9-yl acetic acid ester, swainsonine, lometrexol, dexrazoxane, methioninase, 2′-cyano-2′-deoxy-N4-palmitoyl-1-B-D-arabino furanosyl cytosine and 3-aminopyridine-2-carboxaldehyde thiosemicarbazone.

Examples of monoclonal antibody targeted therapeutic agents include those therapeutic agents which have cytotoxic agents or radioisotopes attached to a cancer cell specific or target cell specific monoclonal antibody. Examples include Bexxar.

“HMG-CoA reductase inhibitors” refers to inhibitors of 3-hydroxy-3-methylglutaryl-CoA reductase. Examples of HMG-CoA reductase inhibitors that may be used include but are not iimited to lovastatin (MEVACOR®; see U.S. Pat. Nos. 4,231,938, 4,294,926 and 4,319,039), simvastatin (ZOCOR®; see U.S. Pat. Nos. 4,444,784, 4,820,850 and 4,916,239), pravastatin (PRAVACHOL®; see U.S. Pat. Nos. 4,346,227, 4,537,859, 4,410,629, 5,030,447 and 5,180,589), fluvastatin (LESCOL®; see U.S. Pat. Nos. 5,354,772, 4,911,165, 4,929,437, 5,189,164, 5,118,853, 5,290,946 and 5,356,896) and atorvastatin (LIPITOR®; see U.S. Pat. Nos. 5,273,995, 4,681,893, 5,489,691 and 5,342,952). The structural formulas of these and additional HMG-CoA reductase inhibitors that may be used in the instant methods are described at page 87 of M. Yalpani, “Cholesterol Lowering Drugs”, Chemistry & Industry, pp. 85-89 (5 Feb. 1996) and U.S. Pat. Nos. 4,782,084 and 4,885,314. The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity, and therefor the use of such salts, esters, open-acid and lactone forms is included within the scope of this invention.

“Prenyl-protein transferase inhibitor” refers to a compound which inhibits any one or any combination of the prenyl-protein transferase enzymes, including farnesyl-protein transferase (FPTase), geranylgeranyl-protein transferase type I (GGPTase-I), and geranylgeranyl-protein transferase type-II (GGPTase-II, also called Rab GGPTase).

Examples of prenyl-protein transferase inhibitors can be found in the following publications and patents: WO 96/30343, WO 97/18813, WO 97/21701, WO 97/23478, WO 97/38665, WO 98/28980, WO 98/29119, WO 95/32987, U.S. Pat. No. 5,420,245, U.S. Pat. No. 5,523,430, U.S. Pat. No. 5,532,359, U.S. Pat. No. 5,510,510, U.S. Pat. No. 5,589,485, U.S. Pat. No. 5,602,098, European Patent Publ. 0 618 221, European Patent Publ. 0 675 112, European Patent Publ. 0 604 181, European Patent Publ. 0 696 593, WO 94/19357, WO 95/08542, WO 95/11917, WO 95/12612, WO 95/12572, WO 95/10514, U.S. Pat. No. 5,661,152, WO 95/1-0515, WO 95/10516, WO 95/24612, WO 95/34535, WO 95/25086, WO 96/05529, WO 96/06138, WO 96/06193, WO 96/16443, WO 96/21701, WO 96/21456, WO 96/22278, WO 96/24611, WO 96/24612, WO 96/05168, WO 96/05169, WO 96/00736, U.S. Pat. No. 5,571,792, WO 96/17861, WO 96/33159, WO 96/34850, WO 96/34851, WO 96/30017, WO 96/30018, WO 96/30362, WO 96/30363, WO 96/31111, WO 96/31477, WO 96/31478, WO 96/31501, WO 97/00252, WO 97/03047, WO 97/03050, WO 97/04785, WO 97/02920, WO 97/17070, WO 97/23478, WO 97/26246, WO 97/30053, WO 97/44350, WO 98/02436, and U.S. Pat. No. 5,532,359. For an example of the role of a prenyl-protein transferase inhibitor on angiogenesis see European J. of Cancer, Vol. 35, No. 9, pp. 1394-1401 (1999).

“Angiogenesis inhibitors” refers to compounds that inhibit the formation of new blood vessels, regardless of mechanism. Examples of angiogenesis inhibitors include, but are not limited to, tyrosine kinase inhibitors, such as inhibitors of the tyrosine kinase receptors Flt-1 (VEGFR1) and Flk-1/KDR (VEGFR2), inhibitors of epidermal-derived, fibroblast-derived, or platelet derived growth factors, MMP (matrix metalloprotease) inhibitors, integrin blockers, interferon-α, interleukin-12, pentosan polysulfate, cyclooxygenase inhibitors, including nonsteroidal anti-inflammatories (NSAIDs) like aspirin and ibuprofen as well as selective cyclooxy-genase-2 inhibitors like celecoxib and rofecoxib (PNAS, Vol. 89, p. 7384 (1992); JNCI, Vol. 69, p. 475 (1982); Arch. Opthalmol., Vol. 108, p. 573 (1990); Anat. Rec., Vol. 238, p. 68 (1994); FEBS Letters, Vol. 372, p. 83 (1995); Clin, Orthop. Vol. 313, p. 76 (1995); J. Mol. Endocrinol., Vol. 16, p. 107 (1996); Jpn. J. Pharmacol., Vol. 75, p. 105 (1997); Cancer Res., Vol. 57, p. 1625 (1997); Cell, Vol. 93, p. 705 (1998); Intl. J. Mol. Med., Vol. 2, p. 715 (1998); J. Biol. Chem., Vol. 274, p. 9116 (1999)), steroidal anti-inflammatories (such as corticosteroids, mineralocorticoids, dexamethasone, prednisone, prednisolone, methylpred, betamethasone), carboxyamidotriazole, combretastatin A-4, squalamine, 6-O-chloroacetyl-carbonyl)-fumagillol, thalidomide, angiostatin, troponin-1, angiotensin II antagonists (see Fernandez et al., J. Lab. Clin. Med. 105:141-145 (1985)), and antibodies to VEGF (see, Nature Biotechnology, Vol. 17, pp. 963-968 (October 1999); Kim et al., Nature, 362, 841-844 (1993); WO 00/44777; and WO 00/61186).

Other therapeutic agents that modulate or inhibit angiogenesis and may also be used in combination with the compounds of the instant invention include agents that modulate or inhibit the coagulation and fibrinolysis systems (see review in Clin. Chem. La. Med. 38:679-692 (2000)). Examples of such agents that modulate or inhibit the coagulation and fibrinolysis pathways include, but are not limited to, heparin (see Thromb. Haemost. 80:10-23 (1998)), low molecular weight heparins and carboxypeptidase U inhibitors (also known as inhibitors of active thrombin activatable fibrinolysis inhibitor [TAFIa]) (see Thrombosis Res. 101:329-354 (2001)). TAFIa inhibitors have been described in PCT Publication WO 03/013,526 and U.S. Ser. No. 60/349,925 (filed Jan. 18, 2002).

“Agents that interfere with cell cycle checkpoints” refer to compounds that inhibit protein kinases that transduce cell cycle checkpoint signals, thereby sensitizing the cancer cell to DNA damaging agents. Such agents include inhibitors of ATR, ATM, the Chk1 and Chk2 kinases and cdk and cdc kinase inhibitors and are specifically exemplified by 7-hydroxystaurosporin, flavopiridol, CYC202 (Cyclacel) and BMS-387032.

“Agents that interfere with receptor tyrosine kinases (RTKs)” refer to compounds that inhibit RTKs and therefore mechanisms involved in oncogenesis and tumor progression. Such agents include inhibitors of c-Kit, Eph, PDGF, Flt3 and c-Met. Further agents include inhibitors of RTKs as described by Blume-Jensen and Hunter, Nature, 411:355-365, 2001.

“Inhibitors of cell-proliferation and survival signaling pathway” refer to pharmaceutical agents that inhibit cell-surface receptors and signal transduction cascades downstream of those surface receptors. Such agents include inhibitors of inhibitors of EGFR (for example gefitinib and erlotinib), inhibitors of ERB-2 (for example trastuzumab), inhibitors of IGFR, inhibitors of cytokine receptors, inhibitors of MET, inhibitors of PI3K (for example LY294002), serine/threonine kinases (including but not limited to inhibitors of Akt such as MK-2206 and those described in WO 02/083064, WO 02/083139, WO 02/083140, US 2004-0116432, WO 02/083138, US 2004-0102360, WO 03/086404, WO 03/086279, WO 03/086394, WO 03/084473, WO 03/086403, WO 2004/041162, WO 2004/096131, WO 2004/096129, WO 2004/096135, WO 2004/096130, WO 2005/100356, WO 2005/100344), inhibitors of Raf kinase (for example BAY-43-9006), inhibitors of MEK (for example CI-1040, AZD6244 and PD-098059) and inhibitors of mTOR (for example Ridaforolimus). Such agents include small molecule inhibitor compounds and antibody antagonists.

“Apoptosis inducing agents” include activators of TNF receptor family members (including the TRAIL receptors).

The invention also encompasses combinations with NSAID's which are selective COX-2 inhibitors. For purposes of this specification NSAID's which are selective inhibitors of COX-2 are defined as those which possess a specificity for inhibiting COX-2 over COX-1 of at least 100 fold as measured by the ratio of IC₅₀ for COX-2 over IC₅₀ for COX-1 evaluated by cell or microsomal assays. Such compounds include, but are not limited to those disclosed in U.S. Pat. No. 5,474,995, U.S. Pat. No. 5,861,419, U.S. Pat. No. 6,001,843, U.S. Pat. No. 6,020,343, U.S. Pat. No. 5,409,944, U.S. Pat. No. 5,436,265, U.S. Pat. No. 5,536,752, U.S. Pat. No. 5,550,142, U.S. Pat. No. 5,604,260, U.S. Pat. No. 5,698,584, U.S. Pat. No. 5,710,140, WO 94/15932, U.S. Pat. No. 5,344,991, U.S. Pat. No. 5,134,142; U.S. Pat. No. 5,380,738, U.S. Pat. No. 5,393,790, U.S. Pat. No. 5,466,823, U.S. Pat. No. 5,633,272, and U.S. Pat. No. 5,932,598, all of which are hereby incorporated by reference.

Inhibitors of COX-2 that are particularly useful in the instant method of treatment are: 3-phenyl-4-(4-(methylsulfonyl)phenyl)-2-(5H)-furanone; and 5-chloro-3-(4-methylsulfonyl)-phenyl-2-(2-methyl-5-pyridinyl)pyridine; or a pharmaceutically acceptable salt thereof.

Compounds that have been described as specific inhibitors of COX-2 and are therefore useful in the present invention include, but are not limited to: parecoxib, CELEBREX® and BEXTRA® or a pharmaceutically acceptable salt thereof.

Other examples of angiogenesis inhibitors include, but are not limited to, endostatin, ukrain, ranpirnase, IM862, 5-methoxy-4-[2-methyl-3-(3-methyl-2-butenyl)oxiranyl]-1-oxaspiro[2,5]oct-6-yl(chloroacetyl)carbamate, acetyldinanaline, 5-amino-1-[[3,5-dichloro-4-(4-chlorobenzoyl)-phenyl]methyl]-1H-1,2,3-triazole-4-carboxamide, CM101, squalamine, combretastatin, RPI4610, NX31838, sulfated mannopentaose phosphate, 7,7-(carbonyl-bis[imino-N-methyl-4,2-pyrrolocarbonylimino[N-methyl-4,2-pyrrole]-carbonylimino]-bis-(1,3-naphthalene disulfonate), and 3-[(2,4-dimethylpyrrol-5-yl)methylene]-2-indolinone (SU5416).

As used above, “integrin blockers” refers to compounds which selectively antagonize, inhibit or counteract binding of a physiological ligand to the αᵥβ₃ integrin, to compounds which selectively antagonize, inhibit or counteract binding of a physiological ligand to the αvβ5 integrin, to compounds which antagonize, inhibit or counteract binding of a physiological ligand to both the αᵥβ₃ integrin and the αᵥβ₅ integrin, and to compounds which antagonize, inhibit or counteract the activity of the particular integrin(s) expressed on capillary endothelial cells. The term also refers to antagonists of the αᵥβ₆, αᵥβ₈, α₁β₁, α₂β₁, α₅β₁, α₆β₁ and α₆β₄ integrins. The term also refers to antagonists of any combination of αᵥβ₃, αᵥβ₅, αᵥβ₆, αᵥβ₈, α₁β₁, α₂β₁, α₅β₁, α₆β₁ and α₆β₄ integrins.

Some specific examples of tyrosine kinase inhibitors include N-(trifluoromethylphenyl)-5-methylisoxazol-4-carboxamide, 3-[(2,4-dimethylpyrrol-5-yl)methylidenyl)indolin-2-one, 17-(allylamino)-17-demethoxygeldanamycin, 4-(3-chloro-4-fluorophenylamino)-7-methoxy-6-[3-(4-morpholinyl)propoxyl]quinazoline, N-(3-ethynylphenyl)-6,7-bis(2-methoxyethoxy)-4-quinazolinamine, BIBX1382,2,3,9,10,11,12-hexahydro-10-(hydroxymethyl)-10-hydroxy-9-methyl-9,12-epoxy-1H-diindolo[1,2,3-fg:3′,2′,1′-kl]pyrrolo[3,4-i][1,6]benzodiazocin-1-one, SH268, genistein, imatinib (STI571), CEP2563, 4-(3-chlorophenylamino)-5,6-dimethyl-7H-pyrrolo[2,3-d]pyrimidinemethane sulfonate, 4-(3-bromo-4-hydroxyphenyl)amino-6,7-dimethoxyquinazoline, 4-(4′-hydroxyphenyl)amino-6,7-dimethoxyquinazoline, SU6668, STI571A, N-4-chlorophenyl-4-(4-pyridylmethyl)-1-phthalazinamine, and EMD121974.

Combinations with compounds other than anti-cancer compounds are also encompassed in the instant methods. For example, combinations of the instantly claimed compounds with PPAR-γ (i.e., PPAR-gamma) agonists and PPAR-δ (i.e., PPAR-delta) agonists are useful in the treatment of certain malingnancies. PPAR-γ and PPAR-δ are the nuclear peroxisome proliferator-activated receptors γ and δ. The expression of PPAR-γ on endothelial cells and its involvement in angiogenesis has been reported in the literature (see J. Cardiovasc. Pharmacol 1998; 31:909-913; J. Biol. Chem. 1999; 274:9116-9121; Invest. Ophthalmol. Vis. Sci. 2000; 41:2309-2317). More recently, PPAR-γ agonists have been shown to inhibit the angiogenic response to VEGF in vitro; both troglitazone and rosiglitazone maleate inhibit the development of retinal neovascularization in mice. (Arch. Ophthamol. 2001; 119:709-717). Examples of PPAR-γ agonists and PPAR-γ/α agonists include, but are not limited to, thiazolidinediones (such as DRF2725, CS-0111, troglitazone, rosiglitazone, and pioglitazone), fenofibrate, gemfibrozil, clofibrate, GW2570, SB219994, AR-H039242, JTT-50-1, MCC-555, GW2331, GW409544, NN2344, KRP297, NP0110, DRF4158, NN622, GI262570, PNU182716, DRF552926, 2-[(5,7-dipropyl-3-trifluoromethyl-1,2-benzisoxazol-6-yl)oxy]-2-methylpropionic acid (disclosed in U.S. Ser. No. 09/782,856), and 2(R)-7-(3-(2-chloro-4-(4-fluorophenoxy)phenoxy)propoxy)-2-ethylchromane-2-carboxylic acid (disclosed in U.S. Ser. No. 60/235,708 and 60/244,697).

Another embodiment of the instant invention is the use of the presently disclosed compounds in combination with gene therapy for the treatment of cancer. For an overview of genetic strategies to treating cancer see Hall et al (Am J Hum Genet. 61:785-789, 1997) and Kufe et al (Cancer Medicine, 5th Ed, pp 876-889, B C Decker, Hamilton 2000). Gene therapy can be used to deliver any tumor suppressing gene. Examples of such genes include, but are not limited to, p53, which can be delivered via recombinant virus-mediated gene transfer (see U.S. Pat. No. 6,069,134, for example), a uPA/uPAR antagonist (“Adenovirus-Mediated Delivery of a uPA/uPAR Antagonist Suppresses Angiogenesis-Dependent Tumor Growth and Dissemination in Mice,” Gene Therapy, August 1998; 5(8):1105-13), and interferon gamma (J Immunol 2000; 164:217-222).

The compounds of the instant invention may also be administered in combination with an inhibitor of inherent multidrug resistance (MDR), in particular MDR associated with high levels of expression of transporter proteins. Such MDR inhibitors include inhibitors of p-glycoprotein (P-gp), such as LY335979, XR9576, OC144-093, R101922, VX853 and PSC833 (valspodar).

A compound of the present invention may be employed in conjunction with anti-emetic agents to treat nausea or emesis, including acute, delayed, late-phase, and anticipatory emesis, which may result from the use of a compound of the present invention, alone or with radiation therapy. For the prevention or treatment of emesis, a compound of the present invention may be used in conjunction with other anti-emetic agents, especially neurokinin-1 receptor antagonists, 5HT3 receptor antagonists, such as ondansetron, granisetron, tropisetron, and zatisetron, GABAB receptor agonists, such as baclofen, a corticosteroid such as Decadron (dexamethasone), Kenalog, Aristocort, Nasalide, Preferid, Benecorten or others such as disclosed in U.S. Pat. Nos. 2,789,118, 2,990,401, 3,048,581, 3,126,375, 3,929,768, 3,996,359, 3,928,326 and 3,749,712, an antidopaminergic, such as the phenothiazines (for example prochlorperazine, fluphenazine, thioridazine and mesoridazine), metoclopramide or dronabinol. In an embodiment, an anti-emesis agent selected front a neurokinin-1 receptor antagonist, a 5HT3 receptor antagonist and a corticosteroid is administered as an adjuvant for the treatment or prevention of emesis that may result upon administration of the instant compounds.

Neurokinin-1 receptor antagonists of use in conjunction with the compounds of the present invention are fully described, for example, in U.S. Pat. Nos. 5,162,339, 5,232,929, 5,242,930, 5,373,003, 5,387,595, 5,459,270, 5,494,926, 5,496,833, 5,637,699, 5,719,147; European Patent Publication Nos. EP 0 360 390, 0 394 989, 0 428 434, 0 429 366, 0 430 771, 0 436 334, 0.443 132, 0 482 539, 0 498 069, 0 499 313, 0 512 901, 0 512 902, 0 514 273, 0 514 274, 0 514 275, 0 514 276, 0 515 681, 0 517 589, 0520 555, 0 522 808, 0 528 495, 0 532 456, 0 533 280, 0 536 817, 0 545 478, 0 558 156, 0 577 394, 0 585 913, 0 590 152, 0 599 538, 0 610 793, 0 634 402, 0 686 629, 0 693 489, 0 694 535, 0 699 655, 0 699 674, 0 707 006, 0 708 101, 0 709 375, 0 709 376, 0 714 891, 0 723 959, 0 733 632 and 0 776 893; PCT International Patent Publication Nos. WO 90/05525, 90/05729, 91/09844, 91/18899, 92/01688, 92/06079, 92/12151, 92/15585, 92/17449, 92/20661, 92/20676, 92/21677, 92/22569, 93/00330, 93/00331, 93/01159, 93/01165, 93/01169, 93/01170, 93/06099, 93/09116, 93/10073, 93/14084, 93/14113, 93/18023, 93/19064, 93/21155, 93/21181, 93/23380, 93/24465, 94/00440, 94/01402, 94/02461, 94/02595, 94/03429, 94/03445, 94/04494, 94/04496, 94/05625, 94/07843, 94/08997, 94/10165, 94/10167, 94/10168, 94/10170, 94/11368, 94/13639, 94/13663, 94/14767, 94/15903, 94/19320, 94/19323, 94/20500, 94/26735, 94/26740, 94/29309, 95/02595, 95/04040, 95/04042, 95/06645, 95/07886, 95/07908, 95/08549, 95/11880, 95/14017, 95/15311, 95/16679, 95/17382, 95/18124, 95/18129, 95/19344, 95/20575, 95/21819, 95/22525, 95/23798, 95/26338, 95/28418, 95/30674, 95/30687, 95/33744, 96/05181, 96/05193, 96/05203, 96/06094, 96/07649, 96/10562, 96/16939, 96/18643, 96/20197, 96/21661, 96/29304, 96/29317, 96/29326, 96/29328, 96/31214, 96/32385, 96/37489, 97/01553, 97/01554, 97/03066, 97/08144, 97/14671, 97/17362, 97/18206, 97/19084, 97/19942 and 97/21702; and in British Patent Publication Nos. 2 266 529, 2 268 931, 2 269 170, 2 269 590, 2 271 774, 2 292 144, 2 293 168, 2 293 169, and 2 302 689. The preparation of such compounds is fully described in the aforementioned patents and publications, which are incorporated herein by reference.

In an embodiment, the neurokinin-1 receptor antagonist for use in conjunction with the compounds of the present invention is selected from: 2-(R)-(1-(R)-(3,5-bis(trifluoromethyl)-phenyl)ethoxy)-3-(S)-(4-fluorophenyl)-4-(3-(5-oxo-1H,4H-1,2,4-triazolo)methyl)morpholine, or a pharmaceutically acceptable salt thereof, which is described in U.S. Pat. No. 5,719,147.

A compound of the instant invention may also be useful for treating or preventing cancer, including bone cancer, in combination with bisphosphonates (understood to include bisphosphonates, diphosphonates, bisphosphonic acids and diphosphonic acids). Examples of bisphosphonates include but are not limited to: etidronate (Didronel) pamidronate (Aredia), alendronate (Fosamax), risedronate (Actonel), zoledronate (Zometa), ibandronate (Boniva), incadronate or cimadronate, clodronate, EB-1053, minodronate, neridronate, piridronate and tiludronate including any and all pharmaceutically acceptable salts, derivatives, hydrates and mixtures thereof.

A compound of the instant invention may also be administered with an agent useful in the treatment of anemia. Such an anemia treatment agent is, for example, a continuous eythropoiesis receptor activator (such as epoetin alfa).

A compound of the instant invention may also be administered with an agent useful in the treatment of neutropenia. Such a neutropenia treatment agent is, for example, a hematopoietic growth factor which regulates the production and function of neutrophils such as a human granulocyte colony stimulating factor, (G-CSF). Examples of a G-CSF include filgrastim.

A compound of the instant invention may also be administered with an immunologic-enhancing drug, such as levamisole, isoprinosine and Zadaxin.

A compound of the instant invention may also be useful for treating or preventing cancer, including bone cancer, in combination with bisphosphonates (understood to include bisphosphonates, diphosphonates, bisphosphonic acids and diphosphonic acids). Examples of bisphosphonates include but are not limited to: etidronate (Didronel), pamidronate (Aredia), alendronate (Fosamax), risedronate (Actonel), zoledronate (Zometa), ibandronate (Boniva), incadronate or cimadronate, clodronate, EB-1053, minodronate, neridronate, piridronate and tiludronate including any and all pharmaceutically acceptable salts, derivatives, hydrates and mixtures thereof.

A compound of the instant invention may also be useful for treating or preventing breast cancer in combination with aromatase inhibitors. Examples of aromatase inhibitors include but are not limited to: anastrozole, letrozole and exemestane.

A compound of the instant invention may also be useful for treating or preventing cancer in combination with siRNA therapeutics.

The compounds of the instant invention may also be administered in combination with γ-secretase inhibitors and/or inhibitors of NOTCH signaling. Such inhibitors include compounds described in WO 01/90084, WO 02/30912, WO 01/70677, WO 03/013506, WO 02/36555, WO 03/093252, WO 03/093264, WO 03/093251, WO 03/093253, WO 2004/039800, WO 2004/039370, WO 2005/030731, WO 2005/014553, U.S. Ser. No. 10/957,251, WO 2004/089911, WO 02/08-1435, WO 02/081433, WO 03/018543, WD 2004/031137, WO 2004/031139, WO 2004/031138, WO 2004/101538, WO 2004/101539 and WO 02/47671 (including LY-450139).

A compound of the instant invention may also be useful for treating or preventing cancer in combination with PARP inhibitors:

A compound of the instant invention may also be useful for treating cancer in combination with the following therapeutic agents: abarelix (Plenaxis Depot®); aldesleukin (Prokine®); Aldesleukin (Proleukin®); Alemtuzumabb (Campath®); alitretinoin (Panretin®); allopurinol (Zyloprim®); altretamine (Hexylen®); amifostine (Ethyol®); anastrozole (Arimidex®); arsenic trioxide (Trisenox®); asparaginase (Elspar®); azacitidine (Vidaza®); bevacuzimab (Avastin®); bexarotene capsules (Targretin®); bexarotene gel (Targretin®); bleomycin (Blenoxane®); bortezomib (Velcade®); busulfan intravenous (Busulfex®); busulfan oral (Myleran®); calusterone (Methosarb®); capecitabine (Xeloda®); carboplatin (Paraplatin®); carmustine (BCNU®, BiCNU®); carmustine (Gliadel®); carmustine with Polifeprosan 20 Implant (Gliadel Wafer®); celecoxib (Celebrex®); cetuximab (Erbitux®); chlorambucil (Leukeran®); cisplatin (Platinol®); cladribine (Leustatin®, 2-CdA®); clofarabine (Clolar®); cyclophosphamide (Cytoxan®, Neosar®); cyclophosphamide (Cytoxan Injection®); cyclophosphamide (Cytoxan Tablet®); cytarabine (Cytosar-U®); cytarabine liposomal (DepoCyt®); dacarbazine (DTIC-Dome®); dactinomycin, actinomycin D (Cosmegen®); Darbepoetin alfa (Aranesp®); daunorubicin liposomal (DanuoXome®); daunorubicin, daunomycin (Daunorubicin®); daunorubicin, daunomycin (Cerubidine®); Denileukin diftitox (Ontak®); dexrazoxane (Zinecard®); docetaxel (Taxotere®); doxorubicin (Adriamycin PFS®); doxorubicin (Adriamycin®, Rubex®); doxorubicin (Adriamycin PFS Injection®); doxorubicin liposomal (Doxil®); DROMOSTANOLONE PROPIONATE (DROMOSTANOLONE®); DROMOSTANOLONE PROPIONATE (MASTERONE INJECTION®); Elliott's B Solution (Elliott's B Solution®); epirubicin (Ellence®); Epoetin alfa (Epogen®); erlotinib (Tarceva®); estramustine (Emcyt®); etoposide phosphate (Etopophos®); etoposide, VP-16 (Vepesid®); exemestane (Aromasin®); Filgrastim (Neupogen®); floxuridine (intraarterial) (FUDR®); fludarabine (Fludara®); fluorouracil, 5-FU (Adrucil®); fulvestrant (Faslodex®); gefitinib (Iressa®); gemcitabine (Gemzar®); gemtuzumab ozogamicin (Mylotarg®); goserelin acetate (Zoladex Implant®); goserelin acetate (Zoladex®); histrelin acetate (Histrelin Implant®); hydroxyurea (Hydrea®); Ibritumomab Tiuxetan (Zevalin®); idarubicin (Idamycin®); ifosfamide (IFEX®); imatinib mesylate (Gleevec®); interferon alfa 2a (Roferon A®); Interferon alfa-2b (Intron A®); irinotecan (Camptosar®); lenalidomide (Revlimid®); letrozole (Femara®); leucovorin (Wellcovorin®, Leucovorin®); Leuprolide Acetate-(Eligard®); levamisole (Ergamisol®); lomustine, CCNU (CeeBU®); meclorethamine, nitrogen mustard (Mustargen®); megestrol acetate (Megace®); melphalan, L-PAM (Alkeran®); mercaptopurine, 6-MP (Purinethol®); mesna (Mesnex®); mesna (Mesnex Tabs®); methotrexate (Methotrexate®); methoxsalen (Uvadex®); mitomycin C (Mutamycin®); mitotane (Lysodren®); mitoxantrone-(Novantrone®); nandrolone phenpropionate (Durabolin-50®); nelarabine (Arranon®); Nofetumomab (Verluma®); Oprelvekin (Neumega®); oxaliplatin (Eloxatin®); paclitaxel (Paxene®); paclitaxel (Taxol®) paclitaxel protein-bound particles (Abraxane®); palifermin (Kepivance®); pamidronate (Aredia®); pegademase (Adagen (Pegademase Bovine)®); pegaspargase (Oncaspar®); Pegfilgrastim (Neulasta®); pemetrexed disodium (Alimta®); pentostatin (Nipent®); pipobroman (Vercyte®); plicamycin, mithramycin (Mithracin®); porfimer sodium (Photofrin®); procarbazine (Matulane®); quinacrine (Atabrine®); Rasburicase (Elitek®); Rituximab (Rituxan®); sargramostim (Leukine®); Sargramostim (Prokine®); sorafenib (Nexavar®); streptozocin (Zanosar®); sunitinib maleate (Sutent®); talc (Sclerosol®); tamoxifen (Nolvadex®); temozolomide (Temodar®); teniposide, VM-26 (Vumon®); testolactone (Teslac®); thioguanine, 6-TG (Thioguanine®); thiotepa (Thioplex®); topotecan (Hycamtin®); toremifene (Fareston®); Tositumomab (Bexxar®); Tositumomab/1-131 tositumomab (Bexxar®); Trastuzumab (Herceptin®); tretinoin, ATRA (Vesanoid®); Uracil Mustard (Uracil Mustard Capsules®); valrubicin (Valstar®); vinblastine (Velban®); vincristine (Oncovin®); vinorelbine (Navelbine®); and zoledronate (Zometa®).

Thus, the scope of the instant invention encompasses the use of the instantly claimed compounds in combination with a second compound selected from: an estrogen receptor modulator, an androgen receptor modulator, retinoid receptor modulator, a cytotoxic/cytostatic agent, an antiproliferative agent, a prenyl-protein transferase inhibitor, an HMG-CoA reductase-inhibitor, an HIV protease inhibitor, a reverse transcriptase inhibitor, an angiogenesis inhibitor, a PPAR-γ agonist, a PPAR-δ agonist, an inhibitor of inherent multidrug resistance, an anti-emetic agent, an agent useful in the treatment of anemia, an agent useful in the treatment of neutropenia, an immunologic-enhancing drug, an inhibitor of cell proliferation and survival signaling, an apoptosis inducing agent, a bisphosphonate, an aromatase inhibitor, an siRNA therapeutic γ-secretase inhibitors, agents that interfere with receptor tyrosine kinases (RTKs), an agent that interferes with a cell cycle checkpoint and any of the therapeutic agents listed above.

Any one or more of the specific dosages and dosage schedules of the compounds of the instant invention, may also be applicable to any one or more of the therapeutic agents to be used in the combination treatment (hereinafter referred to as the “second therapeutic agent”).

Moreover, the specific dosage and dosage schedule of this second therapeutic agent can further vary, and the optimal dose, dosing schedule and route of administration will be determined based upon the specific second therapeutic agent that is being used.

Of course, the route of administration of the compounds of the instant invention is independent of the route of administration of the second therapeutic agent. In an embodiment, the administration for a compound of the instant-invention is oral administration. In another embodiment, the administration for a compound of the instant invention is intravenous administration. Thus, in accordance with these embodiments, a compound of the instant invention is administered orally or intravenously, and the second therapeutic agent can be administered orally, parenterally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecaily, or in a slow release dosage form.

In addition, a compound of the instant invention and second therapeutic-agent may be administered by the same mode of administration, i.e., both agents administered e.g., orally, by IV. However, it is also within the scope of the present invention to administer a compound of the instant invention by one mode of administration, e.g., oral, and to administer the second therapeutic agent by another mode of administration, e.g., IV or any other ones of the administration modes described hereinabove.

The first treatment procedure, administration of a compound of the instant invention, can take place prior to the second treatment procedure, i.e., the second therapeutic agent, after the treatment with the second therapeutic agent, at the same time as the treatment with the second therapeutic agent, or a combination thereof. For example, a total treatment period can be decided for a compound of the instant invention. The second therapeutic agent can be administered prior to onset of treatment with a compound of the instant invention or following treatment with a compound of the instant invention. In addition, anti-cancer treatment can be administered during the period of administration of a compound of the instant invention but does not need to occur over the entire treatment period of a compound of the instant invention.

The term “administration” and variants thereof (e.g., “administering” a compound) in reference to a compound of the invention means introducing the compound or a prodrug of the compound into the system of the animal in need of treatment. When a compound of the invention or prodrug thereof is provided in combination with one or more other active agents (e.g., a cytotoxic agent, etc.), “administration” and its variants are each understood to include concurrent and sequential introduction of the compound or prodrug thereof and other agents.

As used herein, the term “composition” is intended to encompass a product comprising the specified ingredients in the specified amounts, as well as any product which results, directly or indirectly, from combination of the specified ingredients in the specified amounts.

The term “therapeutically effective amount” as used herein means that amount of active compound or pharmaceutical agent that elicits the biological or medicinal response in a tissue, system, animal or human that is being sought by a researcher, veterinarian, medical doctor or other clinician.

The term “treating cancer” or “treatment of cancer” refers to administration to a mammal afflicted with a cancerous condition and refers to an effect that alleviates the cancerous condition by killing the cancerous cells, but also to an effect that results in the inhibition of growth and/or metastasis of the cancer.

In an embodiment, the angiogenesis inhibitor to be used as the second compound is selected from a tyrosine kinase inhibitor, an inhibitor of epidermal-derived-growth factor, an inhibitor of fibroblast-derived growth factor, an inhibitor of platelet-derived growth factor, air MMP (matrix metalloprotease) inhibitor, an integrin blocker, interferon-α, interleukin-12, pentosan polysulfate, a cyclooxygenase inhibitor, carboxyamidotriazole, combretastatin A-4, squalamine, 6-O-chloroacetyl-carbonyl)-fumagillol, thalidomide, angiostatin, troponin-1, or an antibody to VEGF. In an embodiment, the estrogen receptor modulator is tamoxifen or raloxifene.

Also included in the scope of the claims is a method of treating cancer that comprises administering a therapeutically effective amount of a compound of Formula I in combination with radiation therapy and/or in combination with a compound selected from: an estrogen receptor modulator, an androgen receptor modulator, retinoid receptor-modulator, a cytotoxic/cytostatic agent, an antiproliferative agent, a prenyl-protein transferase inhibitor, an HMG-CoA reductase inhibitor, an HIV protease inhibitor, a reverse transcriptase inhibitor, an angiogenesis inhibitor, a PPAR-γ agonist, a PPAR-δ agonist, an inhibitor of inherent multidrug resistance, an anti-emetic agent, an agent useful in the treatment of anemia, an agent useful in the treatment of neutropenia, an immunologic-enhancing drug, an inhibitor of cell proliferation and survival signaling, an apoptosis inducing agent, a bisphosphonate, an aromatase inhibitor, an siRNA therapeutic and an agent that interferes with a cell cycle checkpoint.

And yet another embodiment of the invention is a method of treating cancer that comprises administering a therapeutically effective amount of a compound of Formula I in combination with paclitaxel or trastuzumab.

The invention further encompasses a method of treating or preventing cancer that comprises administering a therapeutically effective amount of a compound of Formula I in combination with a COX-2 inhibitor.

The instant invention also includes a pharmaceutical composition useful for treating or preventing cancer that comprises a therapeutically effective amount of a compound of Formula I and a compound selected from: an estrogen receptor modulator, an androgen receptor modulator, a retinoid receptor modulator, a cytotoxic/cytostatic agent, an antiproliferative agent, a prenyl-protein transferase inhibitor, an HMG-CoA reductase inhibitor, an HIV protease inhibitor, a reverse transcriptase inhibitor, an angiogenesis inhibitor, a PPAR-γ agonist, a PPAR-δ agonist; an inhibitor of cell proliferation and survival signaling, a bisphosphonate, an aromatase inhibitor, an siRNA therapeutic and an agent that interferes with a cell cycle checkpoint.

Further included within the scope of the invention is a method of treating or preventing a disease in which angiogenesis is implicated, which is comprised of administering to a mammal in need of such treatment a therapeutically effective amount of a compound of the present invention. Other inhibitors of MET may also be administered for this method of treatment. Ocular neovascular diseases, which may result in certain forms of blindness, are examples of conditions where much of the resulting tissue damage can be attributed to aberrant infiltration of blood vessels in the eye. The undesirable infiltration can be triggered by ischemic retinopathy, such as that resulting from diabetic retinopathy, retinopathy of prematurity, retinal vein occlusions, etc., or by degenerative diseases, such as the choroidal neovascularization observed in age-related macular degeneration. Inhibiting the growth of blood vessels by administration of the present compounds would therefore prevent the infiltration of blood vessels and prevent or treat diseases where angiogenesis is implicated, such as ocular diseases like retinal vascularization, diabetic retinopathy, age-related macular degeneration, and the like.

Routes of systemic administration of the compounds of the present invention described above may be utilized in the treatment of such ocular neovascular diseases. Other routes of ocular administration may also be employed, such as topical, periocular, intravitreal and the like. Intravitreal implants coated with a drug:polymer matrix may also be employed.

Ophthalmic pharmaceutical compositions that are adapted for topical administration to the eye may be in the form of solutions, suspensions, ointments, creams or as a solid insert. Ophthalmic formulations of this compound may contain from 0.01 ppm to 1% and especially 0.1 ppm to 1% of medicament. For a single dose, from between 0.01 to 5000 ng, preferably 0.1 to 500 ng, and especially 1 to 100 ng of the compound can be applied to the human eye. Formulations useful for intravitreal administration are similar to saline solutions described previously for intravenous administration.

These and other aspects of the invention will be apparent from the teachings contained herein.

### SCHEMES AND EXAMPLES

The compounds of this invention may be prepared by employing reactions as shown in the following schemes, in addition to other standard manipulations that are known in the literature or exemplified in the experimental procedures. The illustrative schemes below, therefore, are not limited by the compounds listed or by any particular substituents employed for illustrative purposes. Substituent numbering as shown in the schemes does not necessarily correlate to that used in the claims and often, for clarity, a single substituent is shown attached to the compound where multiple substituents are allowed under the definitions of the instant invention hereinabove.

Examples provided are intended to assist in a further understanding of the invention. Particular materials employed, species and conditions are intended to be illustrative of the invention and not limiting of the reasonable scope thereof.

The abbreviations used herein have the following tabulated meanings. Abbreviations not tabulated below have their meanings as commonly used unless specifically stated otherwise.

| | |
|---|---|
| Ac = | acetyl |
| Bn = | benzyl |
| Boc = | tert-butyl-carbamate |
| Boc₂O = | Boc-anhydride = di-tert-butyl dicarbonate |
| B₂Pin₂ = | bis(pinacolato)diboron |
| BPIN = | boronic acid, pinacol ester |
| CAMP = | cyclic adenosine-3′,5′-monophosphate |
| CAN = | ceric ammonium nitrate |
| CDI = | carbonyl diimidazole |
| Cs₂CO₃ = | cesium carbonate |
| DAST = | N,N-diethylaminosuflur trifluoride |
| DavePhos = | 2-dicyclohexylphosphino-2′-(N,N- |
| | dimethylamino)biphenyl |
| DBU = | 1,8-diazabicyclo[5.4.0]undec-7-ene |
| DCM = | Dichloromethane = methylene chloride |
| DIAD = | diisopropyl azodicarboxylate |
| DIBAL = | diisobutylaluminum hydride |
| DIPEA = | N-Ethyldiisopropylamine |
| DMA = | N,N-dimethylacetamide |
| DMAP = | 4-(dimethylamino)pyridine |
| DME = | dimethoxyethane |
| DMF = | N,N-dimethylformamide |
| DMFDMA = | 1,1-diethoxy-N,N-dimethylmethanamine |
| DPPA = | diphenylphosphoryl azide |
| EDC = | 3-(ethyliminomethyleneamino)-N,N-dimethyl- |
| | propan-1-amine |
| ESI = | electrospray ionization |
| Et₃N = | triethylamine |
| Et₂O = | diethyl ether |
| EtOAc = | ethyl acetate |
| GST = | glutathione transferase |
| HCl = | hydrochloric acid |
| HMDS = | hexamethyldisilazide |
| Hermann's | trans-di(μ-acetate)bis[o-(di-o- |
| catalyst = | tolylphosphino)benzyl]dipalladium(II) |
| HOBt = | 1-hydroxybenzotriazole |
| HPLC = | high performance liquid chromatography |
| hr = | hour |
| K₂CO₃ = | potassium carbonate |
| KOAc = | potassium acetate |
| KOH = | potassium hydroxide |
| K₃PO₄ = | potassium phosphate tribasic |
| Lawesson's | 2,4-bis(4-methoxyphenyl)-1,3,2,4- |
| reagent = | dithiadiphosphetane 2,4-disulfide |
| LiOH = | lithium hydroxide |
| LDA = | lithium diisopropylamide |
| LRMS = | low resolution mass spectrometry |
| mCPBA = | metachloroperbenzoic acid |
| MMPP = | monoperoxyphthalic acid |
| MPLC = | medium performance liquid chromatography |
| MPPM = | monoperoxyphthalic acid, magnesium salt 6H₂O |
| Ms = | methanesulfonyl = mesyl = SO₂Me |
| MsO = | methanesulfonate = mesylate |
| NaBH₄ = | sodium borohydride |
| Na₂CO₃ = | sodium carbonate |
| NaHCO₃ = | sodium bicarbonate |
| NaOH = | sodium hydroxide |
| n-BuLi = | n-butyl lithium |
| NH₄Cl = | ammonium chloride |
| μW = | microwave |
| NSAID = | non-steroidal anti-inflammatory drug |
| o-Tol = | ortho-tolyl |
| OXONE ® = | 2KHSO₅•KHSO₄•K₂SO₄ |
| PCC = | pyridinium chlorochromate |
| Pd₂(allyl)₂Cl₂ = | allylpalladium (II) chloride dimer |
| PDC = | pyridinium-dichromate |
| PdCl₂(dppf)•DCM = | 1,1′-bis(diphenylphosphino)ferrocene- |
| | palladium (II) dichloride dichloromethane |
| | complex |
| Pd₂(dba)₃ = | dipalladium (0) trisdibenzylideneacetone |
| PDE = | phosphodiesterase |
| Pd(OAc)₂ = | palladium (II) acetate |
| Pd(PPh₃)₄ = | tetrakis(triphenylphosphine)palladium (0) |
| Ph = | phenyl |
| Phe = | benzenediyl |
| PMB = | para-methoxybenzyl |
| Pye = | pyridinediyl |
| r.t. = | room temperature |
| rac- = | racemic |
| RuPhos = | [2′,6′-bis(propan-2-yloxy)biphenyl-2- |
| | yl](dicyclohexyl)phosphane |
| SAM = | aminosulfonyl or sulfonamide or SO₂NH₂ |
| SEM = | 2-(trimethylsilyl)ethoxymethoxy |
| SPA = | scintillation proximity assay |
| T3P = | 2-propanephosphonic anhydride |
| TBAF = | tetra-n-butylammonium fluoride |
| TBTU = | O-(benzotriazol-1-yl)-N,N,N′,N′-tetramethyl- |
| | uronium tetrafluoroborate |
| t-BuLi | t-butyl lithium |
| TBDMS = | tert-butyl(dimethyl)silyl |
| TEA = | triethylamine |
| Th = | 2- or 3-thienyl |
| THP = | tetrahydropyran |
| TFA = | trifluoroacetic acid |
| TFAA = | trifluoroacetic acid anhydride |
| THF = | tetrahydrofuran |
| Thi = | thiophenediyl |
| TLC = | thin layer chromatography |
| TMS-CN = | trimethylsilyl cyanide |
| TMSI = | trimethylsilyl iodide |
| Tz = | 1H (or 2H)-tetrazol-5-yl |
| XPhos = | 2-dicyclohexylphosphino-2′,4′,6′-triisopropyl- |
| | biphenyl |
| C₃H₅ = | allyl |

### Alkyl Group Abbreviations

| | |
|---|---|
| Me = | methyl |
| Et = | ethyl |
| n-Pr = | normal propyl |
| i-Pr = | isopropyl |
| n-Bu = | normal butyl |
| i-Bu = | isobutyl |
| s-Bu = | secondary butyl |
| t-Bu = | tertiary butyl |
| c-Pr = | cyclopropyl |
| c-Bu = | Cyclobutyl |
| c-Pen = | cyclopentyl |
| c-Hex = | cyclohexyl |

### Methods of Synthesis

Substituted aryl or heteroaryl amine I is reacted with sodium nitrite in the presence of aqueous hydrochloric acid as solvent at or around 5° C. to provide a diazonium intermediate that is further reacted with tert-butyl acetoacetate or benzyl acetoacetate in the presence of sodium acetate in a suitable solvent mixture such as ethanol/water at or around 5° C. to afford the corresponding diazo intermediate II. Diazo intermediate II is heated in DMFDMA as solvent at or around 100° C. to afford the corresponding substituted pyridazinone intermediate III (the product wherein R=Me can arise from transesterification). Substituted pyridazinone III (R═^{t}Bu) is treated with an acid such as TFA in a suitable solvent such as DCM to afford the corresponding carboxylic acid intermediate IV. Alternatively, substituted pyridazinone III (R=Me, Bn) is reacted with a nucleophile such as NaI in a solvent such as pyridine at or around 100° C. to afford the corresponding carboxylic acid intermediate IV. The acid IV is then reacted with isobutyl chloroformate in the presence of a suitable base such as N-methyl morpholine in an appropriate solvent such as DCM. The corresponding activated intermediate is then treated with a suitable reducing agent such as sodium borohydride in an appropriate cosolvent such as water at or around 0° C. to afford alcohol intermediate V. Alcohol V is then activated by treating with thionyl chloride in a solvent such as MeCN at or around ambient temperature to afford the corresponding chloride intermediate VI (X═Cl); alternatively, alcohol VI is treated with isobutyl chloroformate in the presence of a suitable base such as pyridine in a solvent such as DCM to afford mixed carbonate VI (X═OCO₂ⁱBu) (Scheme 1).

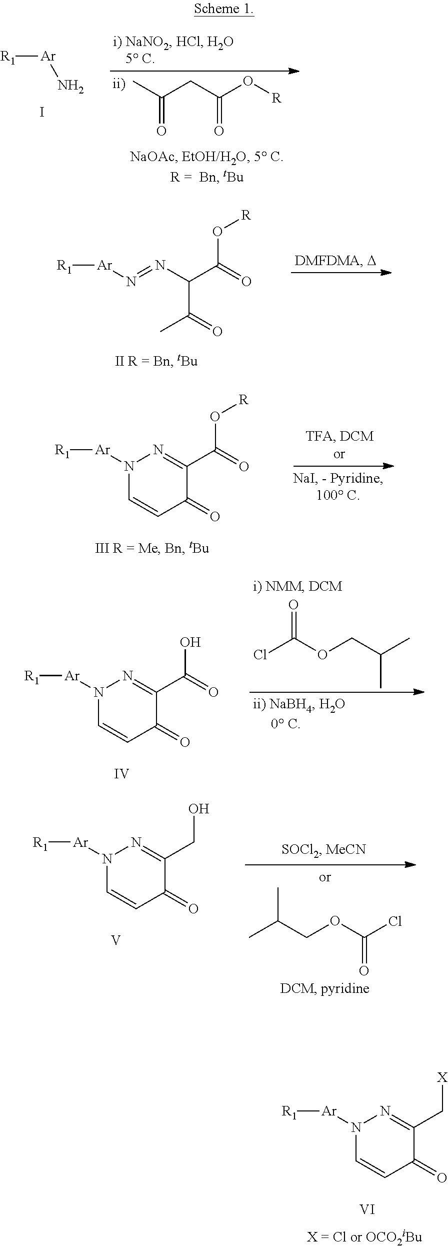

For alcohol intermediates V wherein Ar—R₁ constitutes an aryl bromide (i.e., R₁═Br), the bromide can be converted to a nitrile using zinc (II) cyanide and an appropriate catalyst such as Pd(PPh₃)₄ in a solvent such as DMF, at or around 120° C. under microwave irradiation. Product alcohol V (R₁═CN) is then reacted with thionyl chloride at or around ambient temperature in a suitable solvent such as MeCN to afford chloride intermediate VI (Scheme 2).

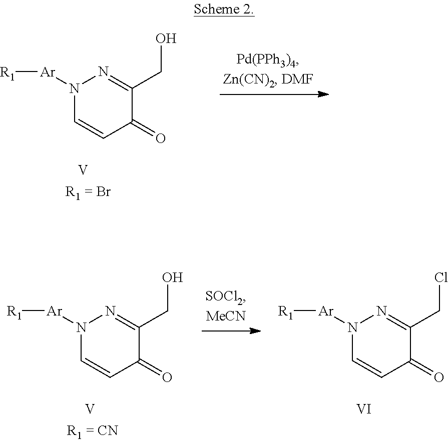

For a specific example wherein I is 1H-pyrazol-4-amine, treatment with sodium nitrite in the presence of aqueous hydrochloric acid as solvent at or around 5° C. provides a diazonium intermediate that is further reacted with tert-butyl acetoacetate in the presence of sodium acetate in a suitable solvent mixture such as ethanol/water at or around 5° C. to afford the corresponding diazo intermediate II. Diazo intermediate II is treated with DMFDMA (as solvent) at or around 90° C. to afford a mixture of tert-butyl ester substituted pyridazinone XIII and methyl ester substituted pyridazinone IX. These two intermediates can be separated by flash chromatography and independently subjected to subsequent transformations. Intermediate XIII (or IX) is reacted with an appropriately substituted alkyl halide in the presence of a base such as Cs₂CO₃ in a suitable solvent (e.g., DMF) under microwave irradiation at or around 110° C. to afford alkylated intermediate III. Alternatively, intermediate VIII (or IX) is reacted with an appropriately substituted alkyl halide in the presence of a base such as NaH in a suitable solvent (e.g., DMF) at or around r.t. to afford alkylated intermediate III. tert-Butyl ester intermediate III is treated with an acid such as TFA in a suitable solvent (e.g., DCM) at or around ambient temperature to afford carboxylic acid intermediate IV. Methyl ester intermediate III is treated with an appropriate base such as LiOH in a solvent system such as THF/water to afford carboxylic acid intermediate IV. The acid IV is then reacted with isobutyl chloroformate in the presence of a suitable base such as N-methyl morpholine in an appropriate solvent such as DCM. The corresponding activated intermediate is then treated with a suitable reducing agent such as sodium borohydride in an appropriate cosolvent such as water at or around 0° C. to afford alcohol intermediate V. Alcohol V is treated with thionyl chloride in a solvent such as MeCN at or around ambient temperature to afford the corresponding chloride intermediate VI (Scheme 3).

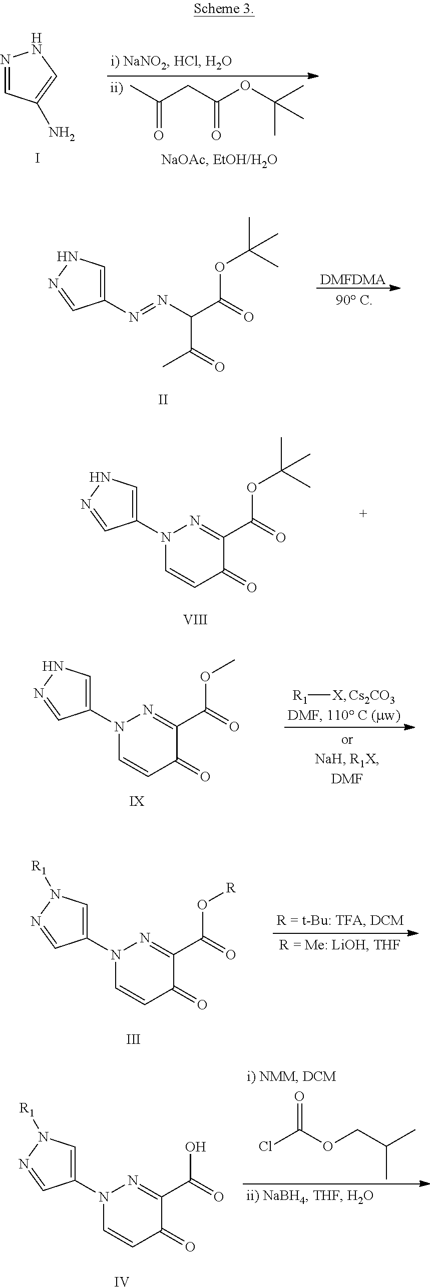

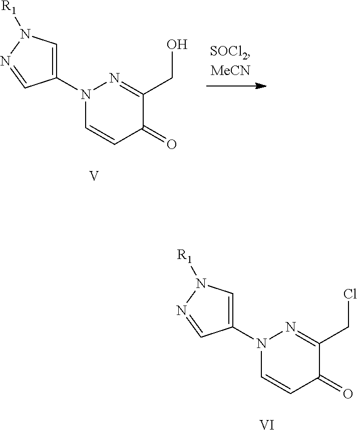

Chloride or mixed carbonate intermediate VI, synthesized according to Schemes 1-3, is reacted with a suitable boronic acid or ester under palladium catalyzed cross-coupling conditions using an appropriate catalyst such as Pd(PPh₃)₄, PdCl₂(dppf).DCM complex or Pd₂ (allyl)₂Cl₂ in the presence of a base such as Na₂CO₃ or K₃PO₄ and an appropriate solvent system such as DME/water or 2-methyl-THF/water at or around 100° C. to provide coupled product VII. Additional transformations to remove one or more protecting groups (such as benzyl, Boc, TBDMS or SEM) and/or hydrogenate an olefin may be performed as required (Scheme 4).

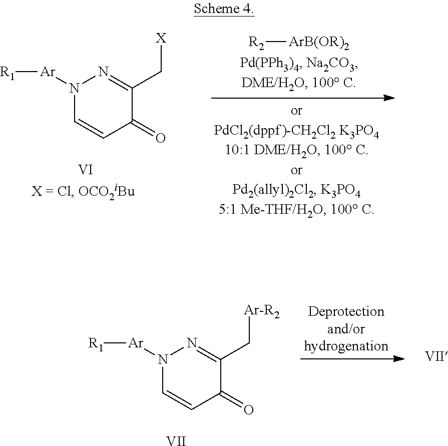

The appropriately substituted boronic esters utilized in the preceding Suzuki coupling reaction (i.e., conversion of VI to VII) may be prepared using the following methods (Boronic ester synthesis A-T):

### Boronic Ester-Synthesis Method A

3-(4,4,5,5-Tetramethyl-1,3,2-dioxaborolan-2-yl)aniline X is treated with an appropriately substituted chloroformate in the presence of a base such as DIPEA in a suitable solvent such as THF at or around 0° C. to afford boronic ester XI. Alternatively, aniline X is treated with disuccinyl carbonate in the presence of an appropriately substituted alcohol using a base such as TEA in a suitable solvent (e.g., MeCN) at or around ambient temperature to afford XI.

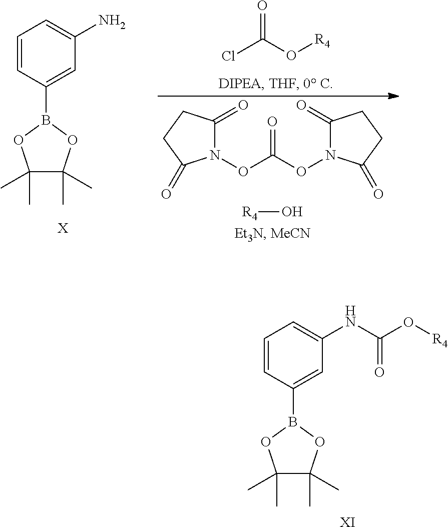

### Boronic Ester Synthesis Method B

Alternatively, commercially available aryl or biaryl halide XII is treated with bis(pinacolato)diboron under palladium catalysis using a palladium/ligand combination such as Pd₂(dba)₃/XPhos as described in Billingsley, K. L.; Barder, T. E.; Buchwald, S. L. Angew. Chem., Int. Ed. 2007, 46, 5359-5363 to afford boronic ester XII. Non-commercial biaryl halides XII may be synthesized by reacting (3-chlorophenyl)boronic acid (or corresponding boronic ester) with an aryl or heteroaryl halide under palladium catalysis (e.g., PdCl₂(dppf).DCM complex) using a base such as K₂CO₃ in a suitable solvent system such as 1,4-dioxane/water at or around 100° C.

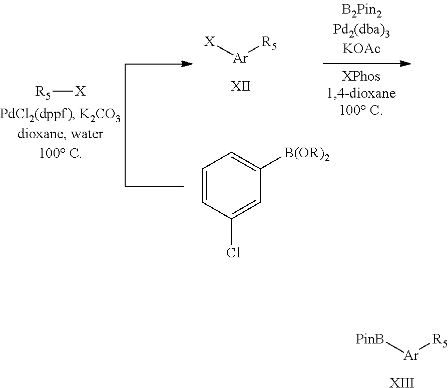

### Boronic Ester Synthesis-Method C

For a specific example wherein XIII is methyl 2-(3-chlorophenyl)pyrimidine-5-carboxylate, reaction with a suitable organometallic reagent such as an alkyl magnesium halide provides tertiary alcohol XIV. Intermediate XIV is then converted to the corresponding boronic ester XV in a similar manner as described above using a palladium/XPhos catalyst system.

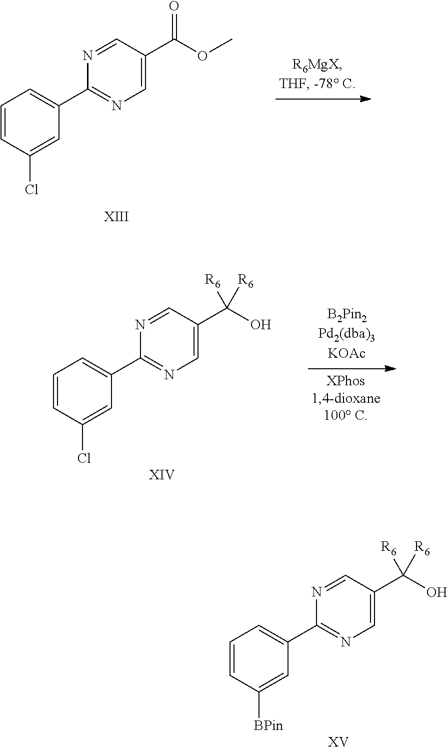

### Boronic Ester Synthesis Method D

In a further example wherein XIII is methyl-2-(3-chlorophenyl)pyrimidine-5-carboxylate, treatment with DIBAL-H at or around 0° C. provides hydroxymethyl intermediate XVI. XVI can be alkylated by reacting with an appropriately substituted alkyl halide in the presence of NaH (or another appropriate base) in a solvent such as DMF at or around ambient temperature or silylated with a silyl chloride such as TBDMS-Cl and base such as imidazole in a solvent such as DMF at or around 80° C. to provide ether or silyl ether XVII. Intermediate XVII is then converted to the corresponding boronic ester XVIII in a similar manner as described above using a palladium/XPhos catalyst-system.

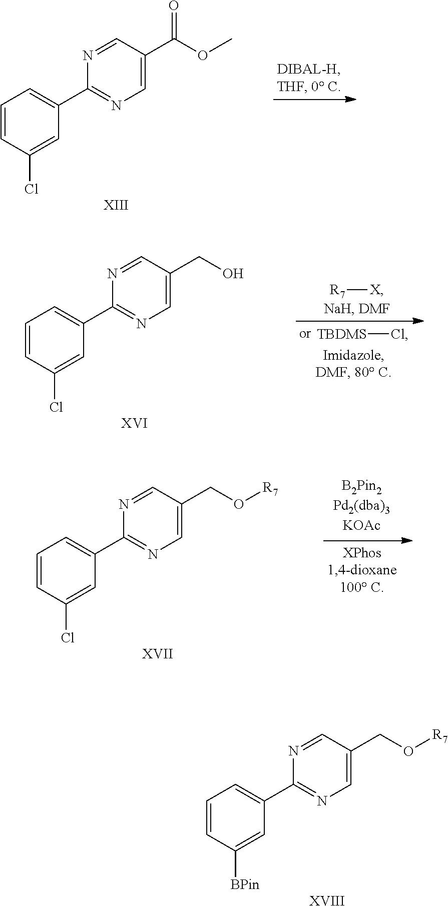

### Boronic Ester Synthesis Method E

Alternatively, hydroxymethyl intermediate XVI can be oxidized to aldehyde XIX using a suitable reagent such as Dess-Martin periodinane in a suitable solvent such as DCM, at or around ambient temperature. Reaction of XIX with a suitable organometallic reagent such as an alkyl magnesium halide provides secondary alcohol XX. XX can be alkylated by reacting with an appropriately substituted alkyl halide in the presence of NaH (or another appropriate base) in a solvent such as DMF at or around ambient temperature to provide ether XXI. Intermediate XXI is then converted to the corresponding boronic ester XXII in a similar manner as described above using a palladium/XPhos catalyst system;

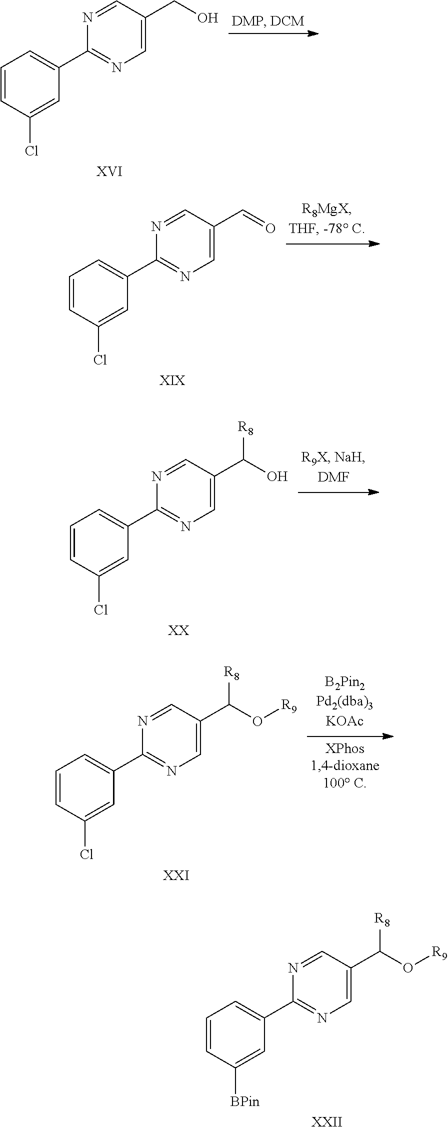

### Boronic Ester Synthesis Method F

Alternatively, reaction of methyl 2-chloropyrimidine-5-carboxylate XXIII with a suitable organometallic reagent such as an alkyl magnesium halide in a suitable solvent such as THF at or around −78° C. provides tertiary alcohol XXIV. XXIV can be alkylated by reacting with an appropriately substituted alkyl halide in the presence of NaH (or another appropriate base) in a solvent such as DMF at or around ambient temperature to provide ether XXV. Intermediate XXV is then reacted with (3-chlorophenyl)boronic acid (or the corresponding boronic ester) in the presence of a suitable palladium catalyst such as PdCl₂(dppf).DCM complex using a base such as Na₂CO₃ in an appropriate solvent system such as 1,4-dioxane/water at or around 100° C. to afford the biaryl intermediate XXVI. Intermediate XXVI is then converted to the corresponding boronic ester XXVII in a similar manner as described above using a palladium/XPhos catalyst system.

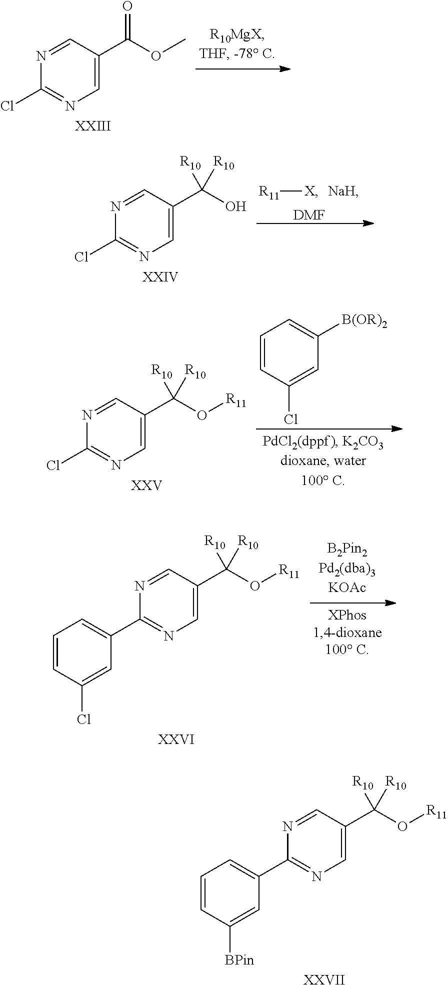

### Boronic Ester Synthesis Method G

Alternatively, 2-bromopyrimidine-5-amine XXVIII is reacted with Boc anhydride in a suitable solvent such as tert-BuOH at or around 60° C. to give Boc-protected aminopyrimidine XXIX. Intermediate XXIX is then reacted with (3-chlorophenyl)boronic acid (or the corresponding boronic ester) in the presence of a suitable palladium catalyst such as PdCl₂(dppf).DCM complex using a base such as K₂CO₃ in an appropriate solvent system such as 1,4-dioxane/water at or around 100° C. to afford the biaryl intermediate XXX. Intermediate XXX is then converted to the corresponding boronic ester XXXI in a similar manner as described above using a palladium/XPhos catalyst system.

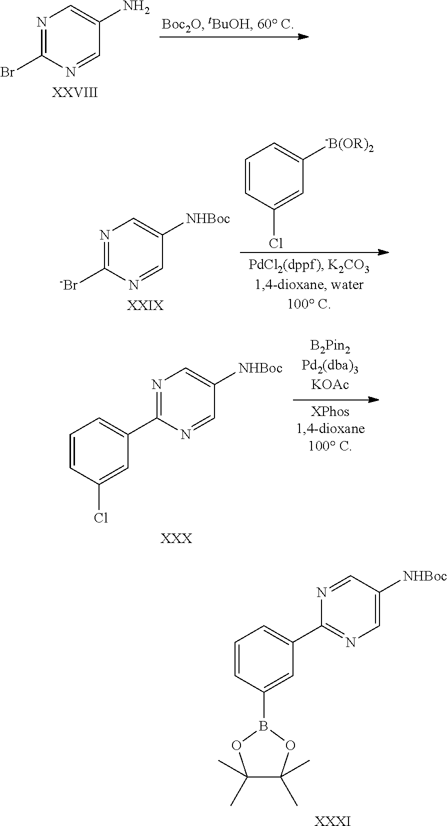

### Boronic Ester Synthesis Method H

Alternatively, 1-chloro-3-iodobenzene XXXII is reacted with an appropriately substituted amide or pyridone in the presence of copper (I) iodide and Cs₂CO₃ in a solvent such as DMSO at or around ambient temperature to afford arylamide XXXIII. XXXIII is converted to boronic ester XXXIV in a similar manner as described above using a palladium/XPhos catalyst system.

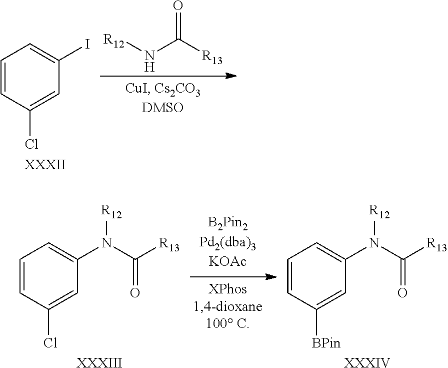

### Boronic Ester Synthesis Method I

Alternatively, 6-chloroquinolin-3-ol XXXV is reacted with an alkyl halide using a base such as NaH in a suitable solvent (e.g., DMF), or is reacted with a substituted alcohol using Mitsunobu conditions (e.g., PPh₃/DIAD in THF solvent) to afford ether XXXVI. Ether XXXVI is then converted to the corresponding boronic ester XXXVII in a similar manner as described above using a palladium/XPhos catalyst system.

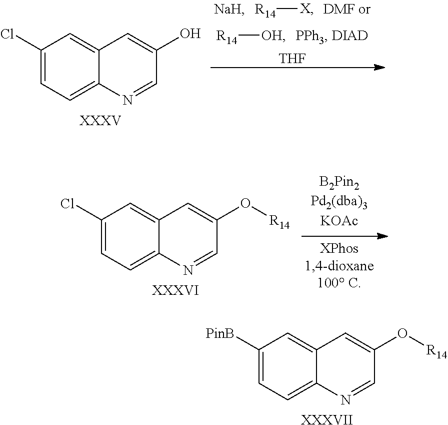

### Boronic Ester Synthesis Method J

Alternatively, 6-bromoquinolin-4-ol XXXVIII is treated with a chlorinating reagent such as POCl₃ at or around ambient temperature to afford 4-chloro-6-bromoquinoline XXXIX. XXXIX is reacted with a metal alkoxide in alcohol solvent at or around 100° C. under microwave irradiation to afford ether XL. XL is then converted to the corresponding boronic ester XLI in a similar manner as described above using a palladium/XPhos catalyst system.

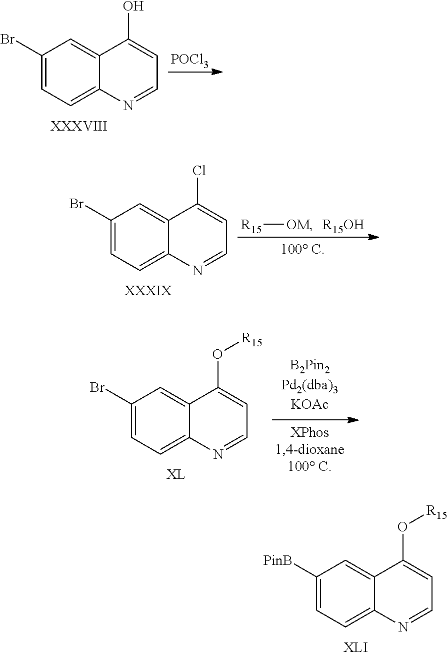

### Boronic Ester Synthesis Method K

Alternatively, 3-bromobenzoic acid XLII is treated with CDI in a solvent such as THF, followed by ammonia gas at <10° C. to give 3-bromobenzamide XLIII. Treatment of benzamide XLIII with DMFDMA at or around 90° C. provides intermediate XLIV. Subsequent cyclization with hydrazine acetate provides 1,2,4-triazole XLV. XLV is alkylated with an appropriately substituted alkyl halide in the presence of NaH (or another appropriate-base) in a solvent such as DMF at or around ambient temperature to afford XLVI. Intermediate XLVI is then converted to the corresponding boronic ester XLVII in a similar manner as described above using a palladium/XPhos catalyst system.

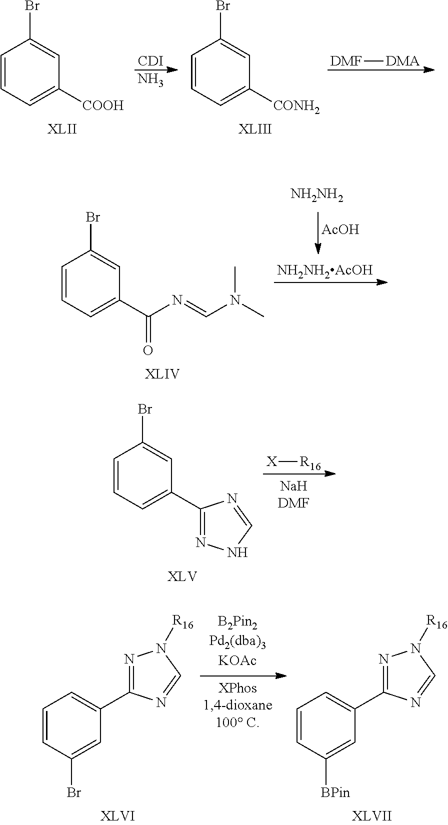

### Boronic Ester Synthesis Method L

Alternatively, 3-bromobenzoic acid XLII is treated with a hydrazide in the presence of a suitable amide coupling reagent system such as EDC/HOBt to give intermediate XLVIII. Treatment of XLVI-III with a suitable thionating reagent such as Lawesson's Reagent in a suitable solvent such as 1,4-dioxane at or around 100° C. provides thiadiazole XLIX. Intermediate XLIX is then converted to the corresponding boronic ester L in a similar manner as described above using a palladium/XPhos catalyst system.

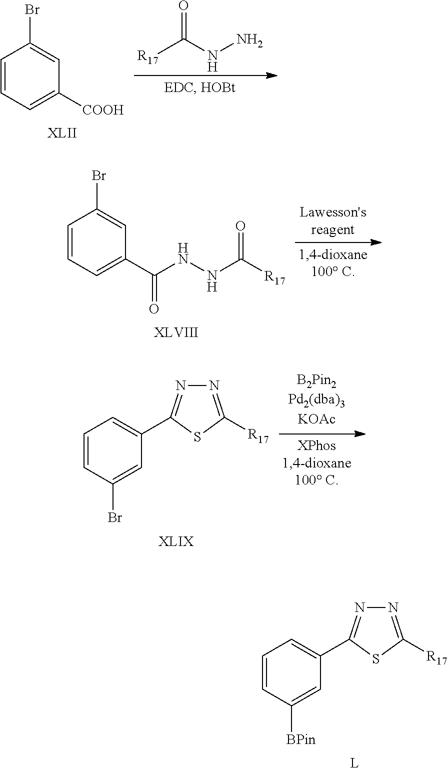

### Boronic Ester Synthesis Method M

Alternatively, 3-bromobenzenecarbothioamide LI is methylated with iodomethane in a suitable solvent such as acetone at or around 50° C. to give methyl 3-bromobenzenecarbimidothioate LII. Treatment of LII with a hydrazide in the presence of ammonium acetate at or around 100° C. provides triazole LIII. Treatment of LIII with SEM-Cl in the presence of a suitable base such as NaH in a suitable solvent such as DMF at or around 50° C. provides SEM-protected triazole LIV. Intermediate LIV is then converted to the corresponding boronic ester LV in a similar manner as described above using a palladium/XPhos catalyst system.

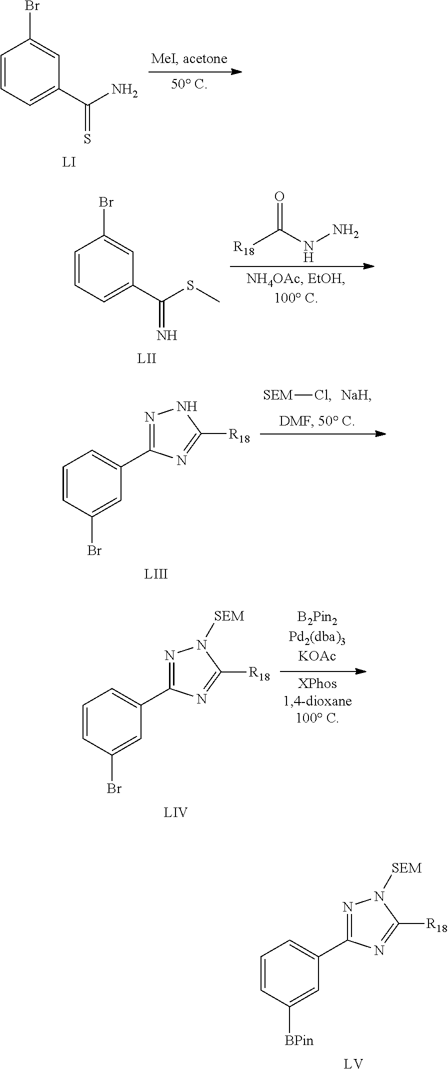

### Boronic Ester Synthesis Method N

Alternatively, 2-chloro-5-bromopyrimidine LVI is treated with n-BuLi at or around −70° C. in the presence of triisopropyl borate to afford boronic acid LVII. Treatment of LVII with a suitable oxidant such as sodium perborate provides 2-chloropyrimidin-5-ol LVIII. LVIII is then reacted with an appropriately substituted alkyl halide in the presence of K₂CO₃ (or another appropriate base) in a solvent such as DMF at or around ambient temperature to as high as 60° C. or is reacted with a substituted alcohol using Mitsunobu conditions (e.g., PPh₃/DIAD in THF solvent) to afford ether intermediate LIX. Intermediate LIX is then reacted with (3-chlorophenyl)boronic acid or a fluorinated (3-chlorophenyl)boronic acid (or the corresponding boronic ester) in the presence of a suitable palladium catalyst such as PdCl₂(dppf).DCM complex using a base such as Na₂CO₃ in an appropriate solvent system (e.g., 1,4-dioxane/water) at or around 100° C. to afford the biaryl intermediate LX. Intermediate LX is then converted to the corresponding boronic ester LXI in a similar manner as described above using a palladium/XPhos catalyst system. Alternatively, LX can be treated with isopropylmagnesium chloride lithium chloride complex in a suitable solvent such as THF at or around −15° C. The arylmagnesium chloride solution is then added to a suitable electrophile such as 2-methoxy-4,4,5,5-tetramethyl-1,3,2-dioxaborolane in a solvent such as THF at or around 0° C. to afford boronic ester LXI.

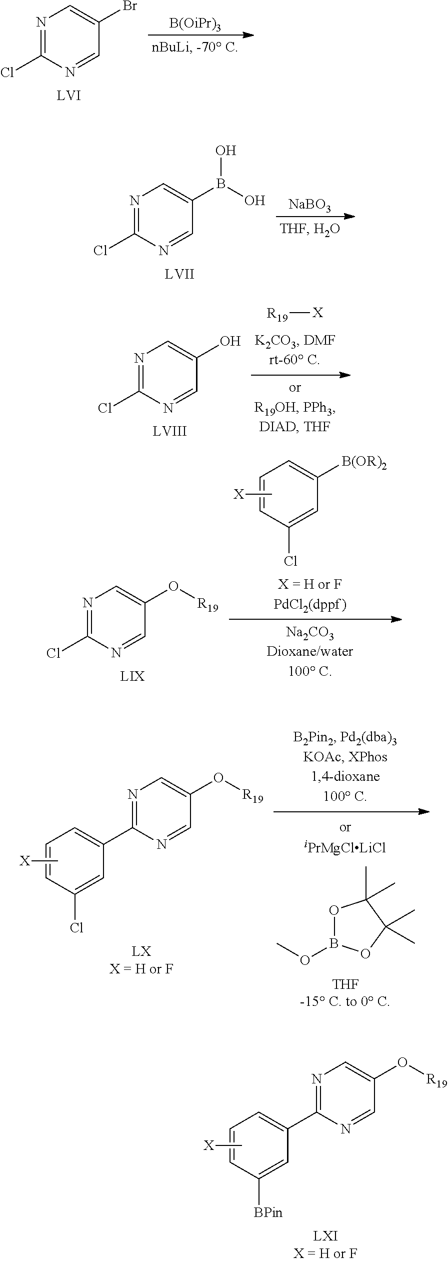

### Boronic Ester Synthesis Method O

Alternatively, biaryl chloride intermediate LX (X═H) is treated with an alkyl lithium followed by oxidative work-up (e.g., CAN) to provide biaryl intermediate LXII. Intermediate LXII is then converted to the corresponding boronic ester LXIII in a similar manner as described above using a palladium/XPhos catalyst system.

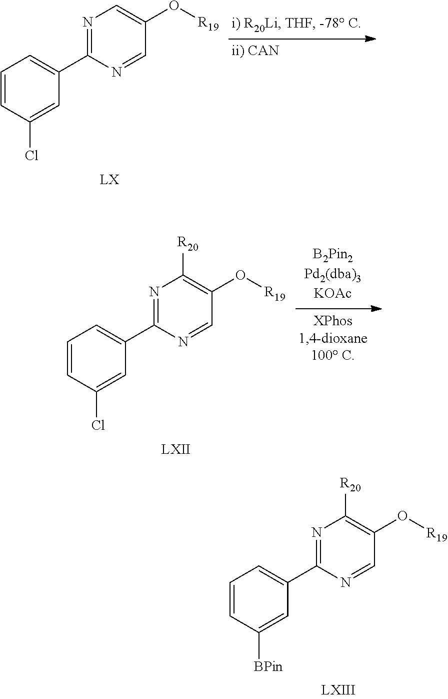

### Boronic Ester Synthesis Method P

For a specific example wherein biaryl chloride-LX is 2-(3-chlorophenyl)-5-(1,4-dioxaspiro[4.5]dec-8-yloxy)pyrimidine, treatment with a suitable acid such as 6 N HCl in a solvent such as THF at or around ambient temperature provides ketone intermediate LXIV. Reaction of LXIV with methylmagnesium bromide provides tertiary alcohol LXV. Intermediate LXV is then converted to the corresponding boronic ester LXVI in a similar manner as described above using a palladium/XPhos catalyst system.

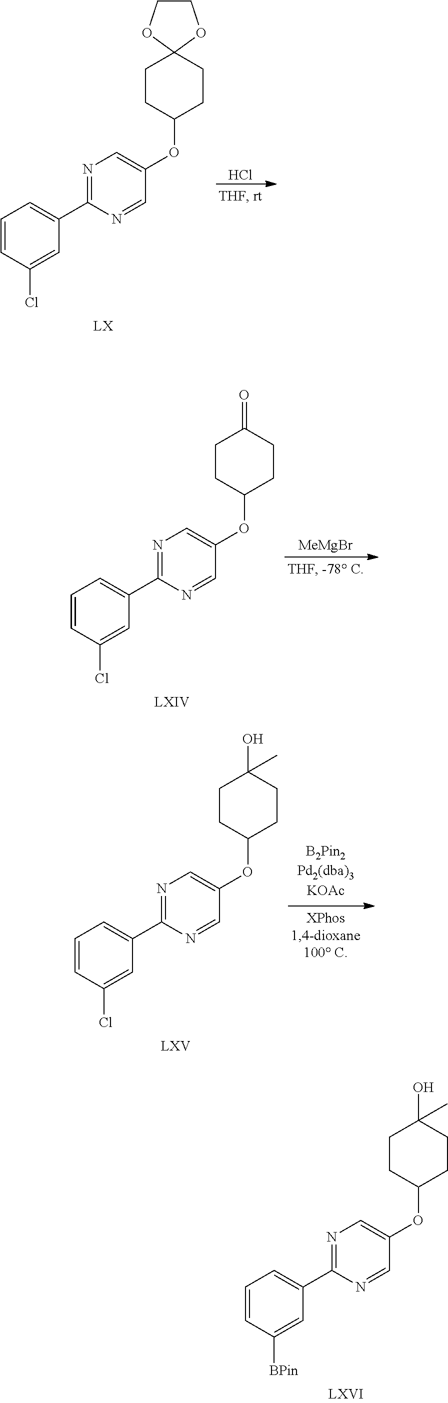

### Boronic Ester Synthesis Method Q

For a specific example wherein biaryl chloride LX is tert-butyl 4-({[2-(3-chlorophenyl)pyrimidin-5-yl]oxy}methyl)piperidine-1-carboxylate, treatment with a suitable hydride source such as DIBAL-H in a solvent such as THF at or around 0° C. up to ambient temperature provides methyl piperidine intermediate LXVII. Intermediate LXVII is then converted to the corresponding boronic ester LXVIII in a similar manner as described above using a palladium/XPhos catalyst system.

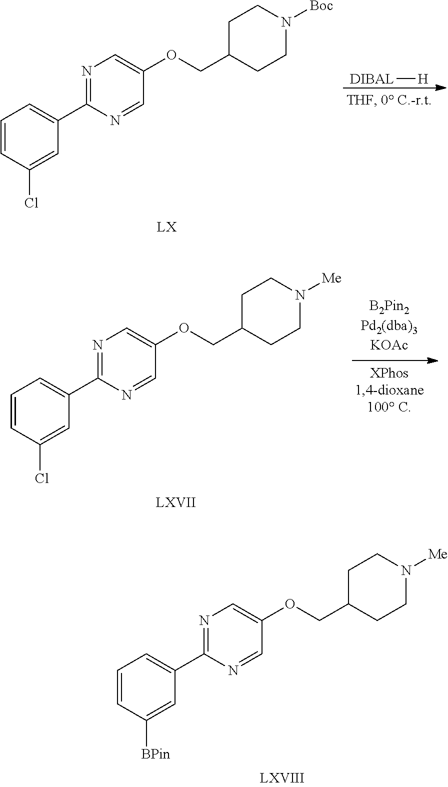

### Boronic Ester Synthesis Method R

Alternatively, 2-chloropyrimidin-5-ol LVII is reacted with an appropriately substituted epoxide in the presence of K₂CO₃ (or another appropriate base) in a solvent such as DMF at or around 50° C. to afford hydroxy intermediate LXIX. Intermediate LXIX is then reacted with (3-chlorophenyl)boronic (or the corresponding boronic ester) in the presence of a suitable palladium catalyst such as PdCl₂(dppf).DCM complex using a base such as Na₂CO₃ in an appropriate solvent system (e.g., 1,4-dioxane/water) at or around 100° C. to afford the biaryl intermediate LXX. Intermediate LXX is then converted to the corresponding boronic ester LXXI in a similar manner as described above using a palladium/XPhos catalyst system.

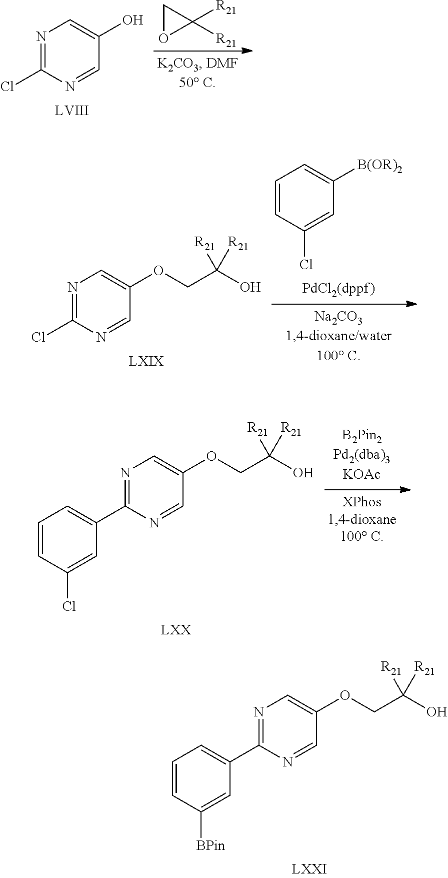

### Boronic Ester Synthesis Method S

Alternatively, 2-chloro-5-bromopyrimidine LVI is reacted with a boronic ester under Suzuki conditions using a suitable catalyst such as Pd(OAc)₂/SPhos or PdCl₂(dppf).DCM complex with a suitable base such as Cs₂CO₃ or K₃PO₄ in a suitable solvent system such as THF/H₂O or 1,4-dioxane/H₂O; or, LVI is reacted with an olefin under Heck conditions using a suitable catalyst such as Pd₂(dba)₃/P^{t} Bu₃.HBF₄ with a suitable base such as N-methyldicycl-ohexylamine in a suitable solvent system such as 1,4-dioxane, to provide functionalized 2-chloropyrimidine LXXI. Intermediate LXXII is then reacted with (3-chlorophenyl)boronic acid (or the corresponding boronic ester) in the presence of a suitable palladium catalyst such as PdCl₂(dppf).DCM complex using a base such as Na₂CO₃ in an appropriate solvent system (e.g., 1,4-dioxane/water) at or around 100° C. to afford the biaryl intermediate LXXIII. Intermediate LXXIII is then converted to the corresponding boronic ester LXXIV in a similar manner as described above using a palladium/XPhos catalyst system.

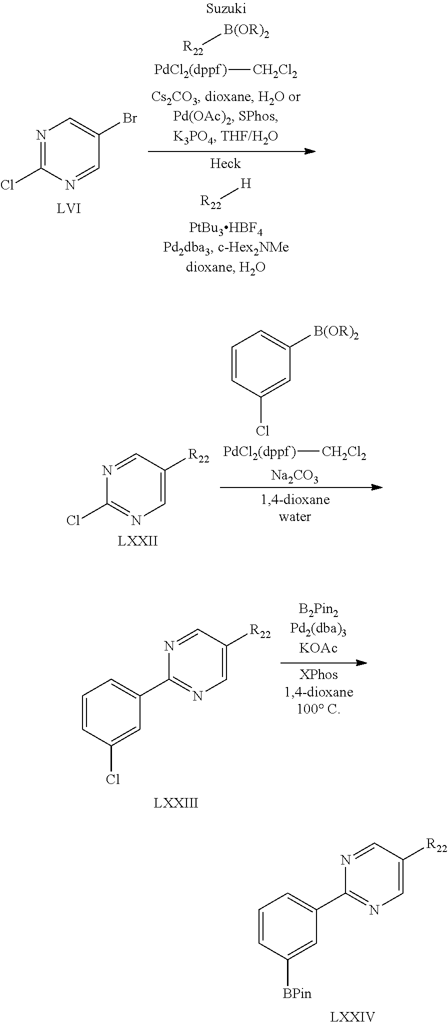

### Boronic Ester Synthesis Method T

Alternatively, 3-bromobenzonitrile LXXV is reacted with TMS-N₃ and dibutyltin oxide in a suitable solvent such as toluene at or around 90° C. to give tetrazole intermediate LXXVI. Treatment of LXXVI with iodomethane in the presence of a suitable base such as KOH in a suitable solvent such as acetone/H₂O at or around ambient temperature provides 2-methyl tetrazole LXXVII (according to the procedures described in WO 9527692). Intermediate LXXVII is then converted to the neopentylglycolato boronic ester LXXVIII using bis(neopentylglycolato)diboron in the presence of a suitable catalyst such as PdCl₂(dppf).DCM and suitable base such as KOAc in a suitable solvent such as 1,4-dioxane/DMSO at or around 90° C. (according to the procedure described in WO 03006464).

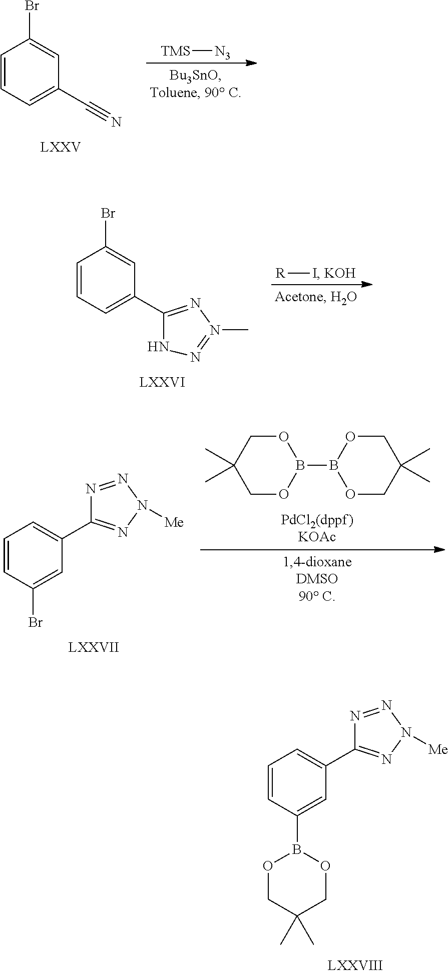

Substituted arylmethyl pyridazinone VII, LXXX, LXXXI, LXXXII, LXXXII, LXXXV, XCV, XCVI or XCVII (vide infra) can be alkylated using an appropriately substituted alkyl halide in the presence of a suitable base such as NaH in a solvent such as DMF or DMF/THF at or around ambient temperature to give LXXIX. An additional transformation to remove a protecting group (such as benzyl or TBDMS) may be performed as required. Separation of enantiomers A and B can be achieved (before or after deprotection) using preparative chiral supercritical fluid chromatography (Scheme 5).

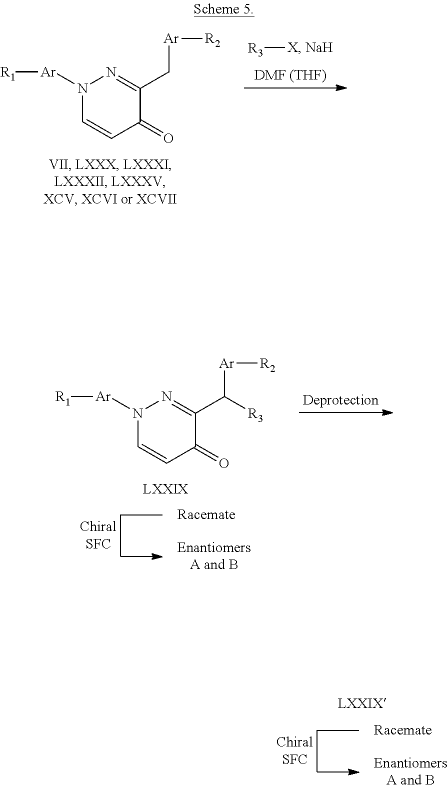

Hydroxypyrimidine intermediate VII′ (R₃═H) or LXXIX′ (R₃=Me, racemate or single enantiomers) is treated with an appropriately substituted epoxide in the presence of a suitable base (e.g., K₂CO₃) in a solvent such as DMF at or around 100° C. (microwave heating) to afford the corresponding alcohol LXXX.

Alternatively, hydroxypyrimidine intermediate VII′ or LXXIX′ are reacted with an appropriately substituted alkyl halide in the presence of a suitable base (e.g., K₂CO₃ or Cs₂CO₃) in a solvent such as DMF at or around r.t. to as high as 150° C. (conventional or microwave heating) with or without the use of tetrabutylammonium iodide as catalyst to afford LXXXI. An additional transformation to remove a protecting group (such as Boc) or saponify an ester may be performed as required.

Alternatively, hydroxypyrimidine intermediate VII′ or LXXIX′ can be difluoromethylated by reacting with 2-chloro-2,2-difluoro-1-phenylethanone in the presence of KOH in a solvent such as MeCN to give LXXXII (as described in J. Hu et al, J. Org. Chem., 20406, 71, 9845). Separation of individual stereoisomers can be achieved (before or after deprotection) using preparative chiral supercritical fluid chromatography (Scheme 6).

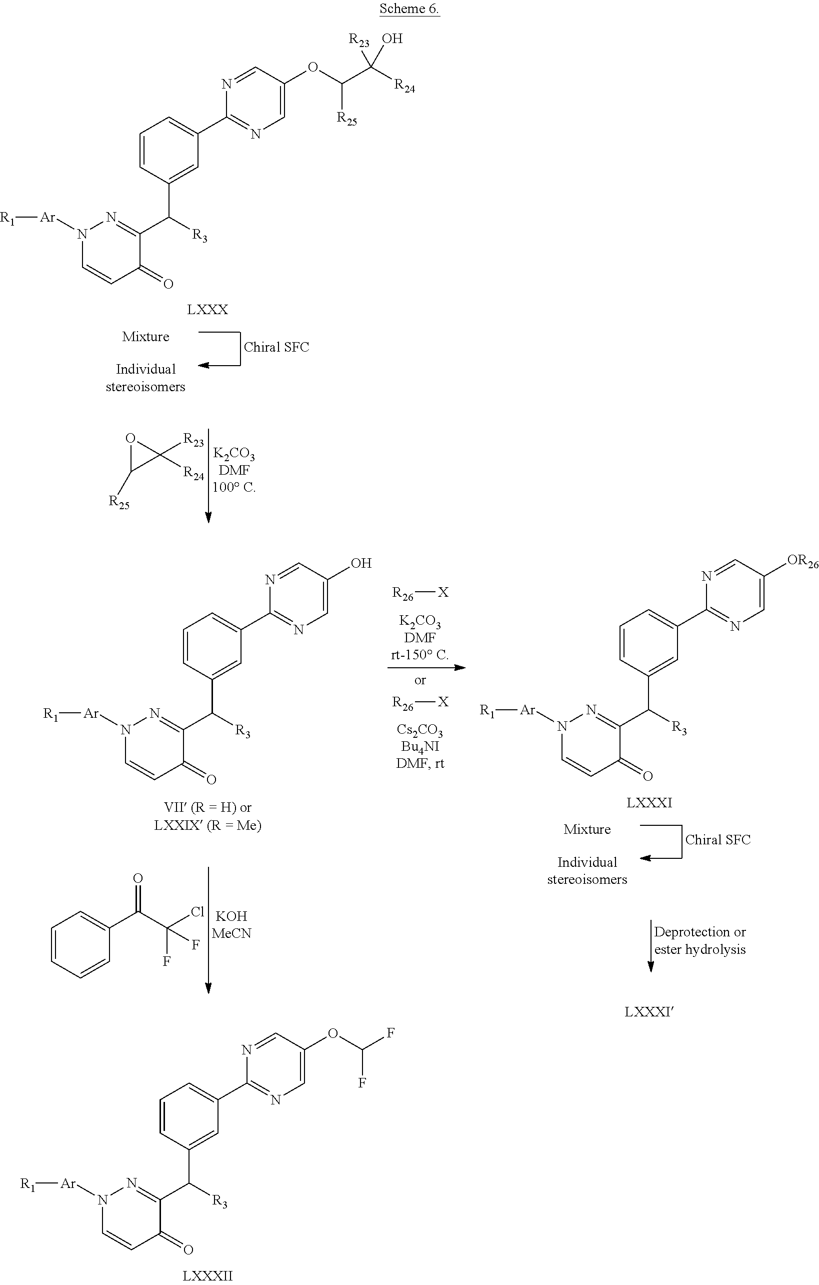

Hydroxy ethers LXXX and LXXXI (R₃═H) can be converted to the corresponding fluoro ethers LXXXIII by treatment with DAST in a suitable solvent such as DCM at or around 0° C. (Scheme 7).

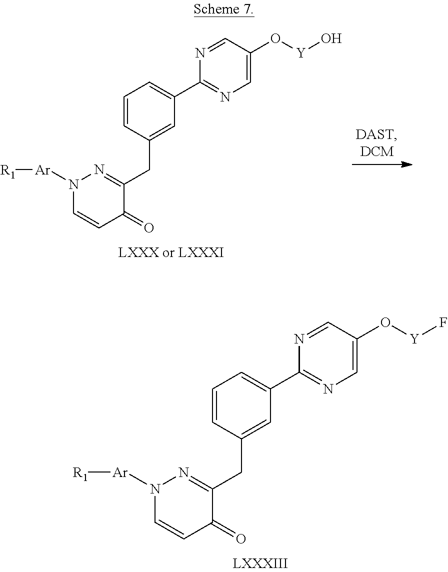

Alternatively, hydroxypyrimidine intermediate VII′ (R₃═H) or LXXIX′ (R₃=Me, single enantiomers) is treated with a triflating reagent such as 1,1,1-trifluoro-N-phenyl-N-[(trifluoromethyl)sulfonyl]methanesulfonamide in the presence of a base such as DIPEA in a solvent such as THF to provide triflate-LXXXIV. LXXXIV can be reacted with a substituted potassium trifluoroborate salt using a suitable catalyst system such as PdCl₂(dppf).DCM complex or Pd₂(dba)₃/XPhos in the presence of a suitable base such as Cs₂CO₃ in a suitable solvent system such as toluene/H₂O or THF/H₂O to give LXXXV. An additional transformation to remove a protecting group (such as Boc) or hydrogenate an olefin may be performed as required.

Alternatively, triflate LXXXIV can be reacted with a substituted boronic acid or ester using a suitable catalyst system such as Pd₂(dba)₃/RuPhos in the presence of a suitable base such as K₃PO₄ in a suitable solvent system such as 1,4-dioxane/H₂O to give LXXXVI.

Alternatively, triflate LXXXIV can be reacted with an alkylzinc iodide (generated from an alkyl iodide and zinc dust) using a suitable catalyst system such as Pd(OAc)₂/RuPhos in a suitable solvent system such as THF to give LXXXVII.

Alternatively, triflate LXXXIV can be reacted with a substituted amine in a suitable solvent system such as NMP at 200° C. under microwave irradiation to give LXXXVIII.

Alternatively, triflate LXXXIV is converted to the corresponding boronic acid LXXXIX in a similar manner as described above (for boronic ester synthesis Method B) using a palladium/XPhos catalyst system. Boronic acid LXXXIX is then reacted with an appropriately substituted-aryl halide under palladium catalyzed cross-coupling conditions using a catalyst/ligand system such as Pd2(dba)₃/XPhos in the presence of a suitable base such as CS₂CO₃ in a solvent such as 1,4-dioxane at or around 100° C. to afford biaryl XC (Scheme 8).

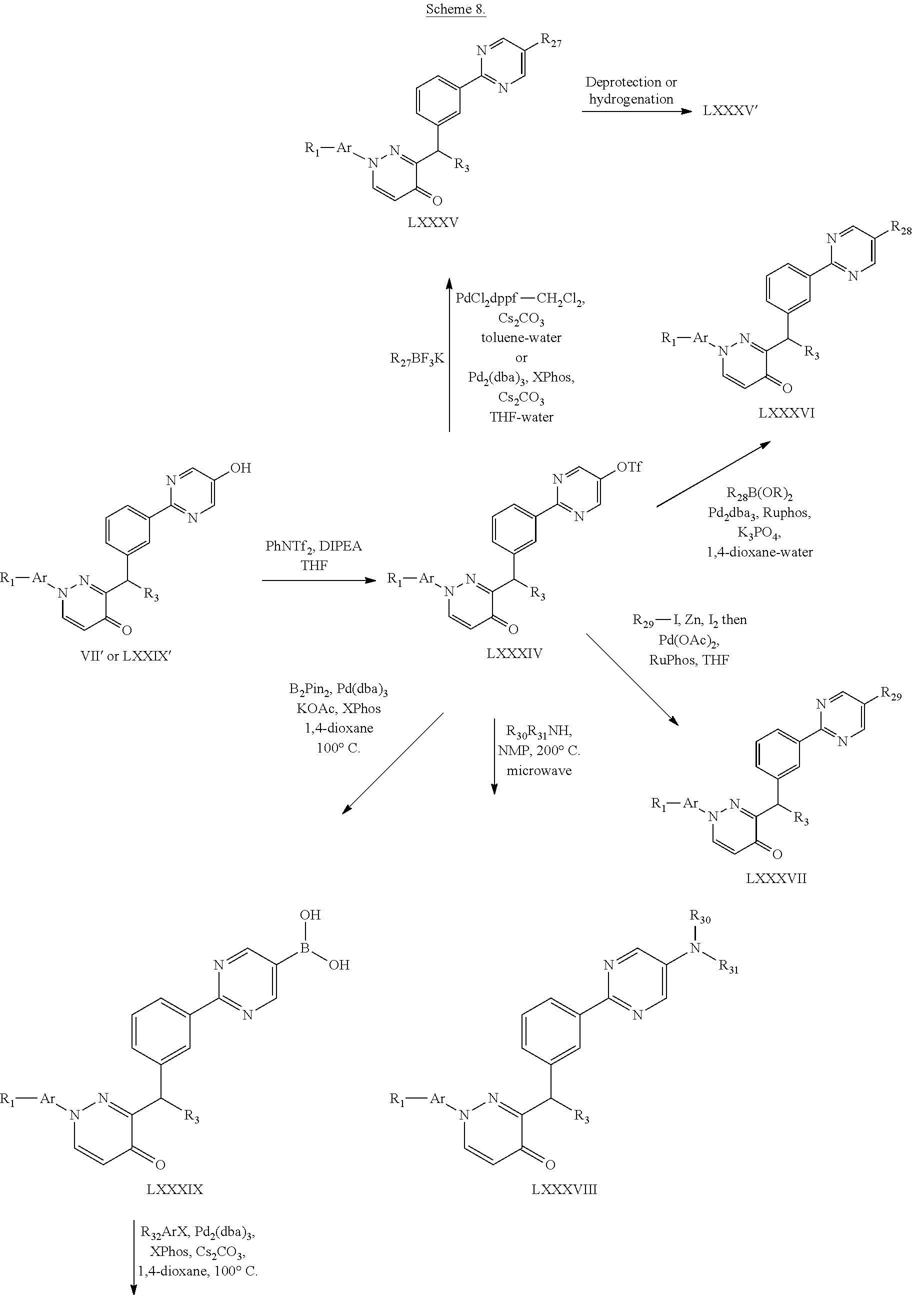

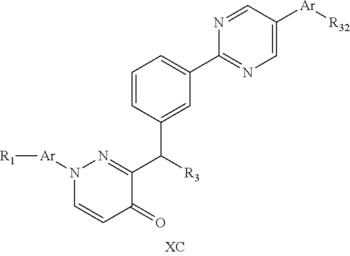

Boc-protected amino pyrimidine VII can be alkylated with a substituted alkyl halide in the presence of a suitable base such as Cs₂CO₃ in a suitable solvent such as DMF at or around 80° C. to give Boc-protected alkylamine XCI. Removal of the Boc group under acidic conditions (e.g., TFA/DCM) provides alkylamine XCII. XCII can be methylated using formaldehyde in the presence of a reducing agent such as NaBH₃CN in a suitable solvent such as AcOH/MeCN at or around 20° C. to give dialkylamine XCIH (Scheme 9).

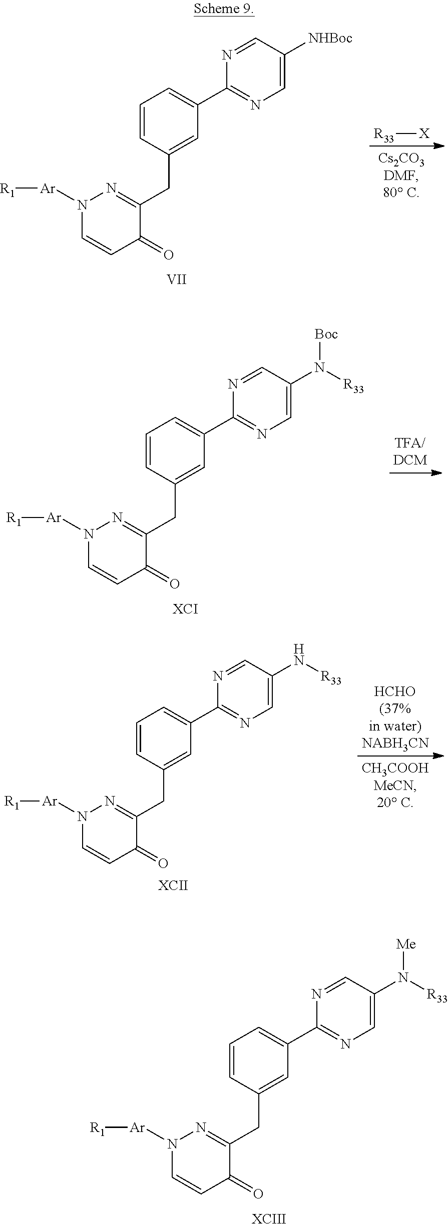

Amino pyrimidine VII′ is reacted with a carboxylic acid in the presence of a suitable amide coupling reagent such as T3P in the presence of a suitable base such as DIPEA in a solvent such as DMF to give amide XCIV.

Alternatively, amino pyrimidine VII′ is treated with tert-butyl nitrite in the presence of copper (I) bromide in a solvent such as MeCN to give bromo pyrimidine XCV. Bromide XCV can be further reacted with a substituted amine in the presence of a suitable catalyst system such as Pd₂(dba)₃/DavePhos in the presence of a suitable base such as sodium tert-butoxide in a solvent such as toluene to give substituted amino pyrimidine XCVI.

Alternatively, amino pyrimidine VII′ is treated with N′-[(1E)-(dimethylamino)methylene]-N,N-dimethylhydrazonoformamide in the presence of a suitable acid catalyst such as PTSA in a solvent such as toluene at or around 100° C. to give 1,3,4-triazole XCVII (Scheme 10).

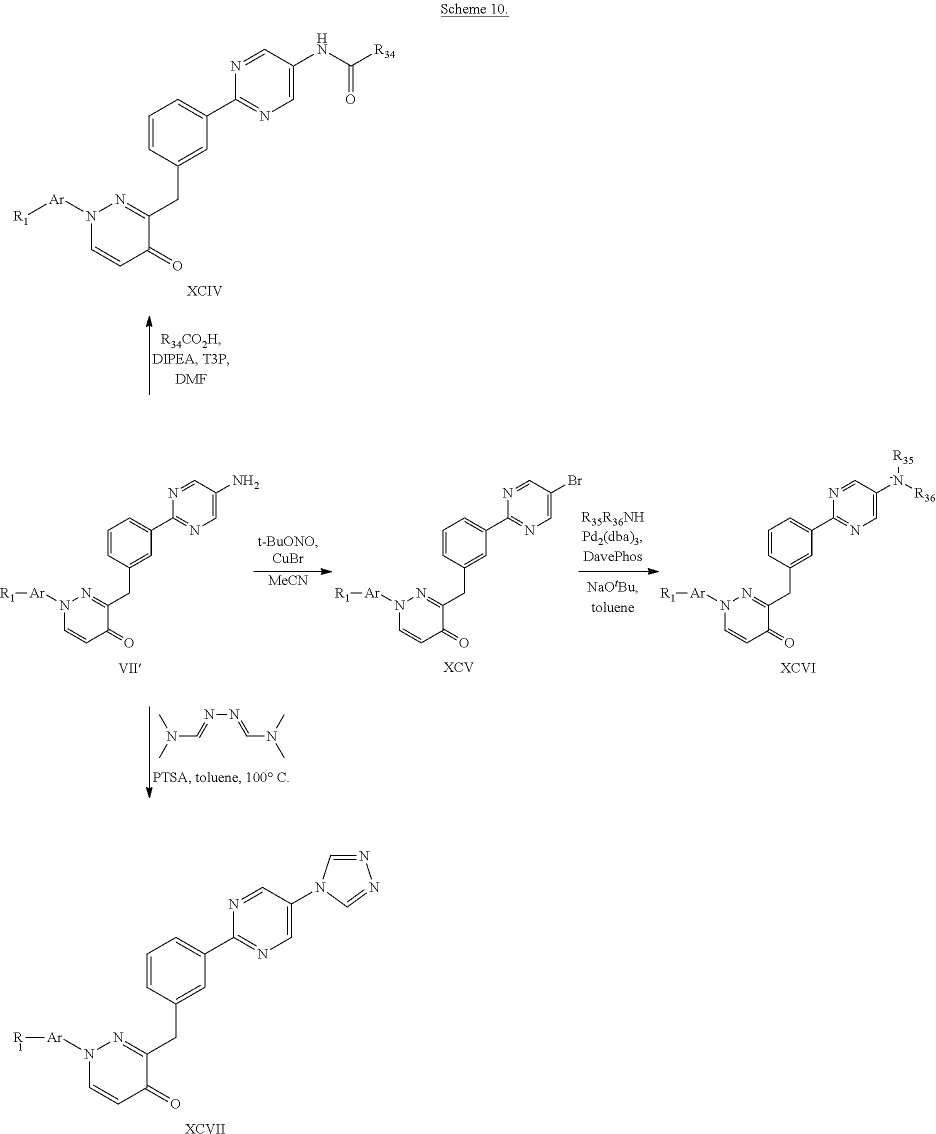

Pyrimidine ester VII is hydrolyzed under basic conditions (e.g., aqueous NaOH) in a suitable solvent such as MeOH under microwave irradiation to give carboxylic acid XCVIII. XCVIII is then reacted with a substituted amine in the presence of a suitable amide coupling reagent system such as Si-carbodiimide/HOBt in the presence of a suitable base such as DIPEA in a solvent such as DMF under microwave irradiation to give amide XCIX (Scheme 11).

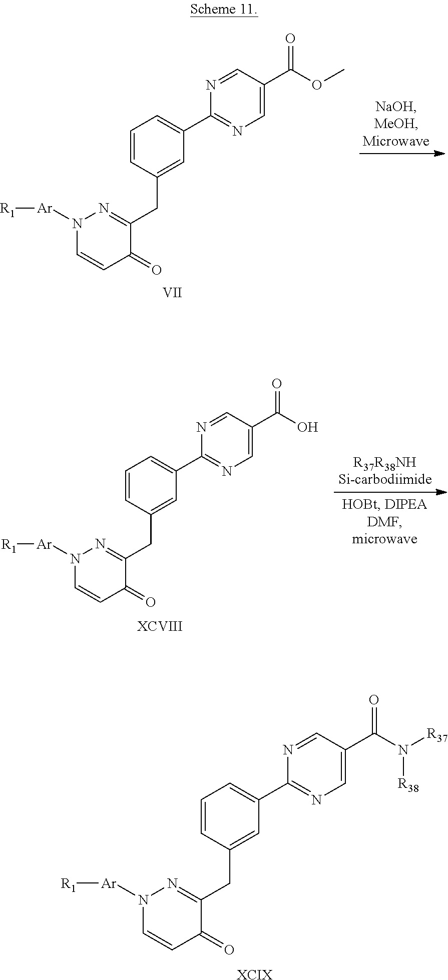

An appropriately substituted alcohol is reacted with disuccinyl carbonate in the presence of a suitable base such as Et₃N in a solvent system such as MeCN/DMSO at or around ambient temperature to afford a mixed carbonate intermediate that is then reacted with aniline intermediate VII′ to afford the corresponding carbamate C. Alternatively, aniline VII′ is reacted with an appropriately substituted chloroformate in the presence of a base such as DIPEA in a suitable solvent (e.g., DCM) at or around ambient temperature to afford the corresponding carbamate C. An additional transformation to remove a protecting group (such as Boc or TBDMS) or saponify an ester may be performed as required.

Alternatively, aniline intermediate VII′ is reacted with 2-chloroethyl chloridocarbonate in the presence of a suitable base such as potassium carbonate in a solvent such as acetonitrile at or around ambient temperature to afford the corresponding oxazolidinone CI.

Alternatively, aniline VII′ is reacted with 4-bromobutanoyl chloride in the presence of a suitable base (e.g., Et₃N) in an appropriate solvent system such as DCM/THF at or around ambient temperature to afford the corresponding lactam CII.

Alternatively, aniline VII′ is reacted with an appropriately substituted isocyanate in a solvent such as THF at or around ambient temperature to afford the corresponding urea CIII. Alternatively, the requisite isocyanate is prepared by reacting an appropriately substituted carboxylic acid with diphenylphosphoryl azide (DPPA) in the presence of a base such as DIPEA in a suitable solvent (e.g., THF) at or around 90° C.

Alternatively, aniline VIP is reacted with an appropriately substituted acid chloride in the presence of a suitable base such as DIPEA in a solvent such as THF at or around ambient temperature to afford amide CIV (Scheme 12).

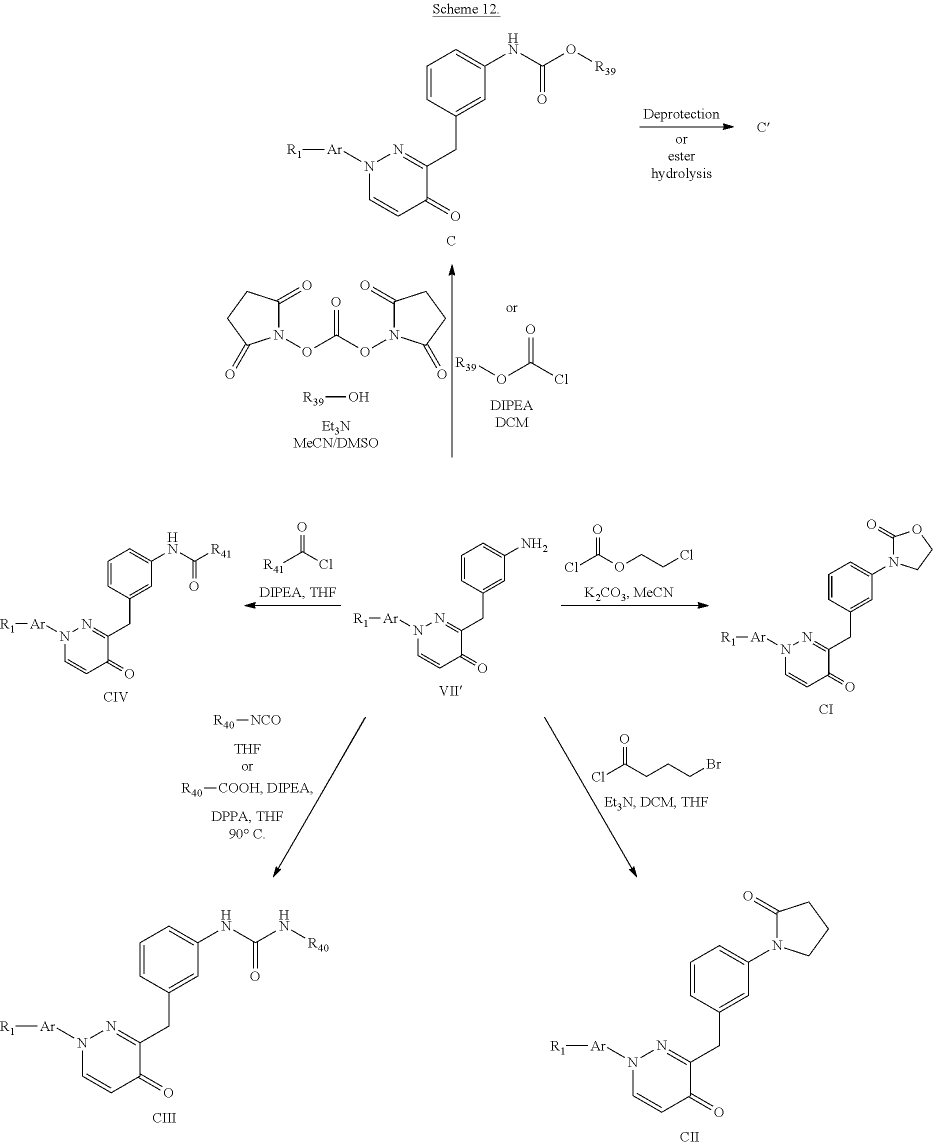

Alternatively, aniline VII′ is prepared by first reacting carboxylic acid intermediate IV with N-methoxymethanamine hydrochloride under amide coupling conditions such as EDC/HOBt using a base such as DIPEA in an appropriate solvent (e.g., DMF) at or around ambient temperature to afford Weinreb amide intermediate CV. Intermediate CV is then reacted with an appropriately substituted aryl Grignard reagent (e.g., {3-[bis(trimethylsilyl)amino]phenyl}magnesium chloride) in a solvent such as THF at or around −78° C. followed by an acidic quench (i.e., HCl) to afford the corresponding ketone CVI. Reduction of the ketone CVI under hydrogenation/hydrogenolysis conditions (i.e., H₂, balloon pressure or above; 10 wt % Pd(OH)₂/C catalyst) in a suitable solvent such as MeOH at or around 50° C. affords aniline VII′.

### Alternative Aniline Synthesis

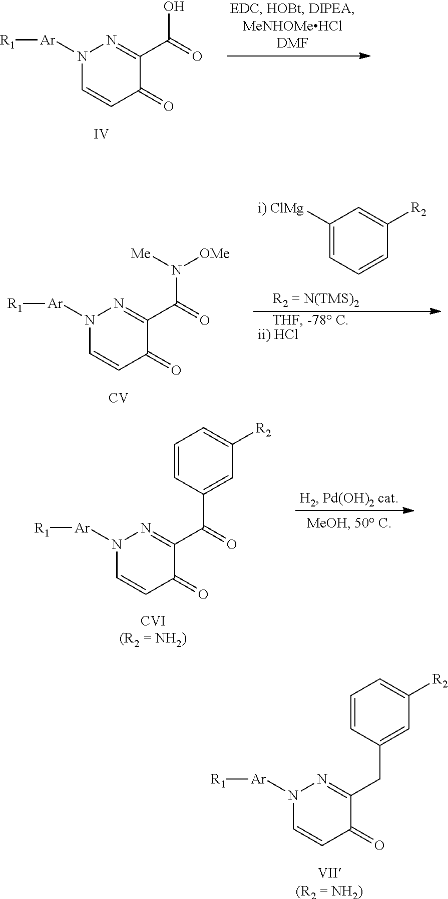

Mono or difluoro substituted amine hydrochloride C′ is reacted with 1,1′-oxybis(2-bromoethane) in the presence of a suitable base such as DIPEA in a solvent such as DMF at or around 65° C. to afford the corresponding substituted morpholine CVII (Scheme 13).

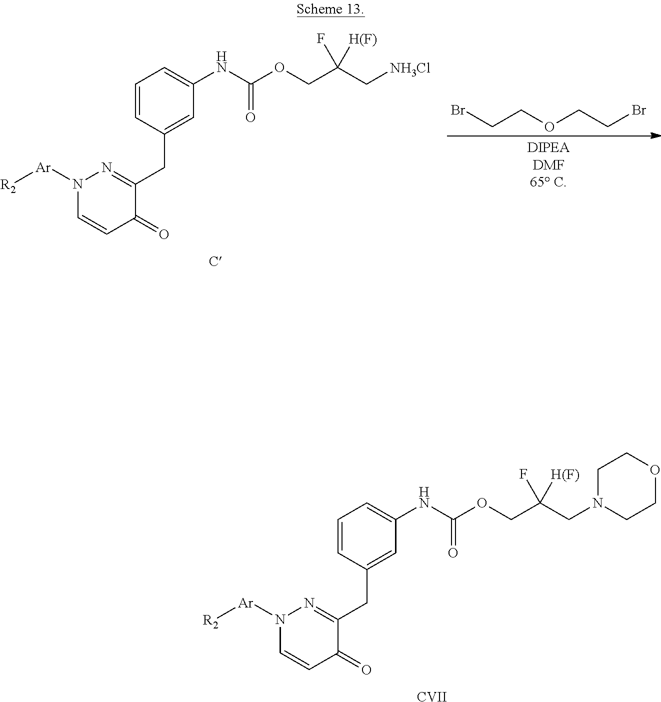

Aniline intermediate CVI is reacted with an appropriately substituted chloroformate in the presence of a suitable base such as DIPEA in a solvent such as THF at or around ambient temperature to afford benzoyl carbamate CVIII. Carbamate CVIII is treated with hydrogen (at balloon pressure or above) using an appropriate palladium catalyst such as 10 wt % Pd/C or Pd(OH)₂/C in a suitable solvent such as MeOH or MeOH/DMA to give benzyl carbamate CIX. Simultaneous removal of a benzyl protecting group may also be achieved.

Alternatively, CVIII is reacted with a reducing reagent such as sodium borohydride in a solvent such as MeOH at or around ambient temperature to afford alcohol intermediate CXI. Alternatively, aniline CVI is reduced using hydrogen at balloon pressure or above in the presence of a suitable palladium catalyst such as 10 wt % Pd/C in a solvent such as MeOH at or around ambient temperature to afford the corresponding alcohol CX. Alcohol CX is then treated with an appropriately substituted chloroformate in the presence of a suitable base such as DIPEA in a solvent such as THF at or around ambient temperature to afford intermediate alcohol CXI. Alcohol CXI is then treated with hydrogen (at balloon pressure or above) using an appropriate palladium catalyst such as 10 wt % Pd/C in a suitable solvent such as MeOH/DMA at or around ambient temperature to afford benzyl carbamate CIX.

Alternatively, alcohol CX is reacted with DAST in a solvent such as DCM at or around ambient temperature to afford the corresponding fluoro compound CXII (Scheme 14).

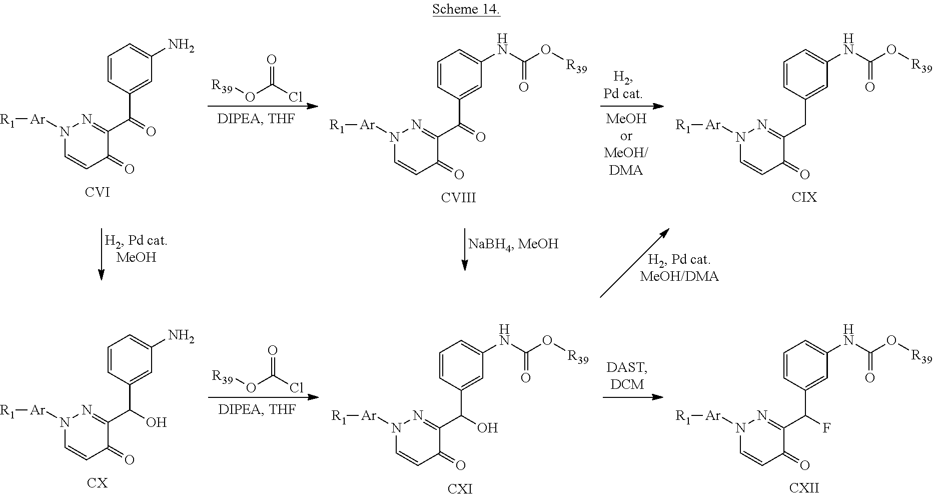

Alcohol CIX is reacted with DPPA under Mitsunobu conditions (e.g., PPh₃/DIAD) in a suitable solvent such as THF at or around 0° C. to ambient temperature to afford azide CXIII. Azide CXIII is then converted to amine CXIV using hydrogen at balloon pressure or above with an appropriate palladium catalyst (e.g., 10 wt % Pd/C) in a solvent such as EtOH at or around ambient temperature (Scheme 15).

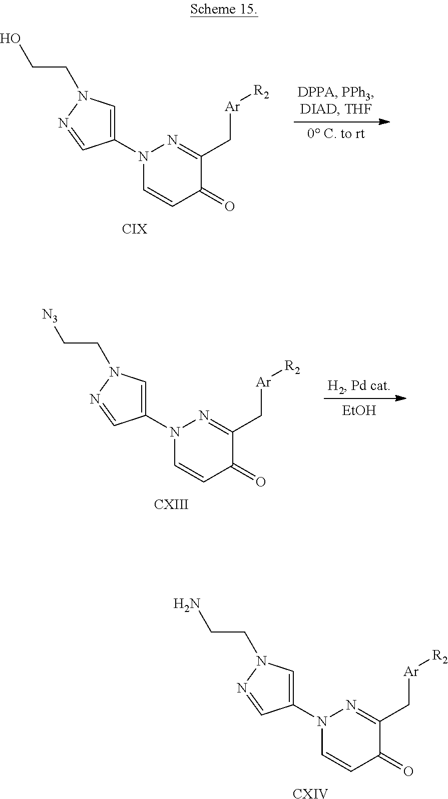

Phenol CIX is methylated with a reagent such as dimethylsulfate in the presence of a suitable base such as Cs₂CO₃ in a solvent such as DMF to afford CXV (where R₄₂=Me). Alternatively, CIX is reacted with an appropriately substituted alkyl halide using a base such as Cs₂CO₃ in a solvent such as DMF at or around 120° C. under microwave irradiation to afford CXV (Scheme 16).

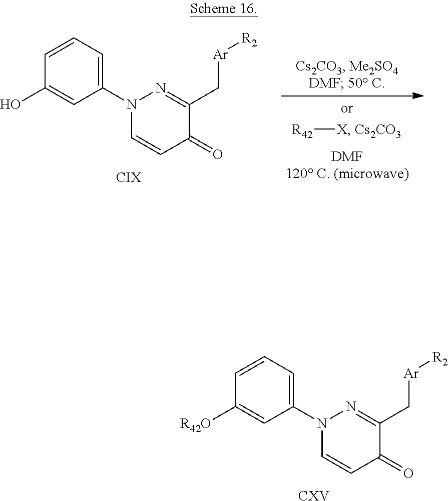

Weinreb amide intermediate CV is treated with methylmagnesium iodide in a solvent such as THF at or around −78° C. to afford methyl ketone CXVI. Ketone CXVI is then treated with an appropriately functionalized-aryl Grignard (i.e., {3-[bis(trimethylsilyl)amino]phenyl}magnesium chloride) in a suitable solvent such as THF at or around −78° C. Following an acidic quench (i.e., HCl) and treatment with TFA/DCM, intermediate CXVII is obtained. Intermediate CXVII is treated with an appropriately substituted chloroformate in the presence of a base such as DIPEA in a solvent such as THF at or around ambient temperature to afford carbamate CXVIII. In the case where R₁Ar is a 4-chlorophenyl moiety (i.e., R₁═Cl), intermediate CXVIII is treated with hydrogen at balloon pressure or above using an appropriate palladium catalyst such as 10 wt % Pd/C in suitable solvent system such as MeOH/DMA to afford the des-chloro intermediate CXVIII (i.e., R₁═H). Intermediate CXVIII is then treated with an acid such as TFA in a suitable solvent such as DCM at or around 50° C. to afford alkene intermediate CXIX. Alkene intermediate CXIX is reduced under hydrogenation conditions using hydrogen at balloon pressure or above in the presence of an appropriate palladium catalyst such as 100 wt % Pd/C in a solvent system such as MeOH/DMA at or around ambient temperature to afford CXX (Scheme 17).

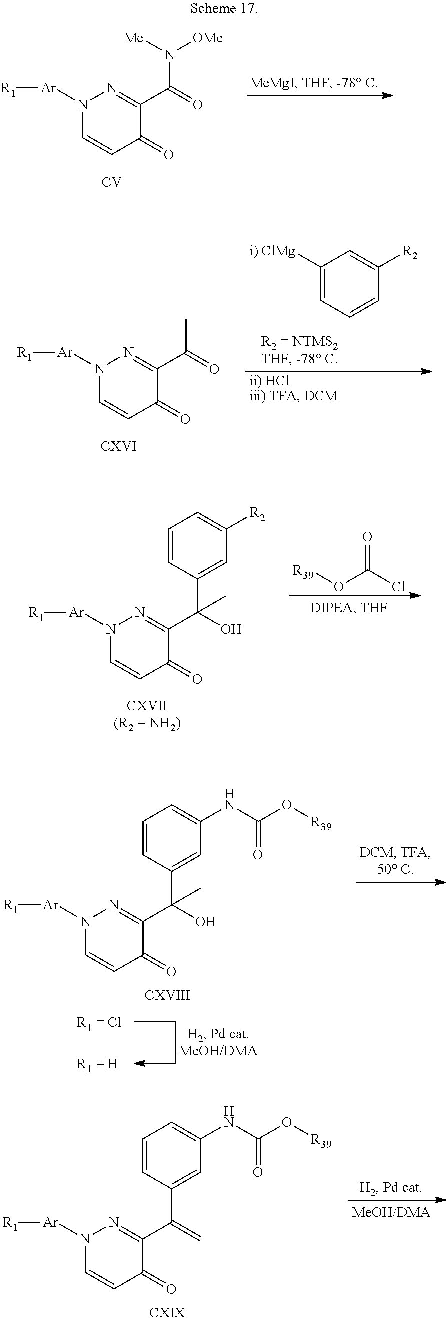

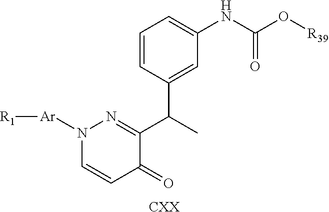

Nitro-intermediate LXXIX (R₂═NO₂) can be reduced under hydrogenation conditions using hydrogen at balloon pressure or above in the presence of an appropriate palladium catalyst such as Pt+V/C in a solvent system such as MeOH at or around ambient temperature to afford aniline CXXI (R₂═NH₂). Aniline CXXI is treated with an appropriately substituted chloroformate in the presence of a base such as DIPEA in a solvent such as THF at or around ambient temperature to afford carbamate CXXII. Separation of enantiomers A and B can be achieved using preparative chiral supercritical fluid chromatography (Scheme 18).

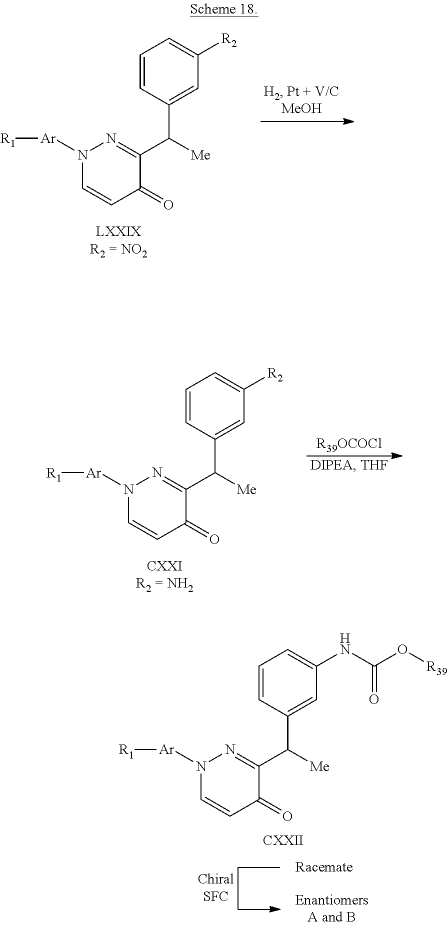

Weinreb amide CV is reacted with 3-chlorophenylmagnesium chloride in a suitable solvent such as THF at or around 0° C. to give aryl chloride intermediate CXXIII. Intermediate CXXIII is then converted to the corresponding boronic ester CXXIV in a similar manner as described above (for boronic ester synthesis Method B) using a palladium/XPhos catalyst system. Boronate CXXIV is reacted with an aryl halide in the presence of a suitable catalyst such as PdCl₂(dppf).DCM in the presence of a suitable base such as Na₂CO₃ in a solvent such as 1,4-dioxane at or around 100° C. to give biaryl ketone intermediate CXXV. Treatment of CXXV with a hydride source such as NaBH₄ in a solvent such as MeOH at or around ambient temperature gives hydroxyl intermediate CXXVI. Reaction of alcohol CXXVI with DAST in a solvent such as DCM at or around ambient temperature provides fluoro product CXXVII. Separation of enantiomers A and B can be achieved using preparative chiral supercritical fluid chromatography.

Alternatively, alcohol CXXVI can be alkylated with an appropriately substituted alkyl halide in the presence of a suitable base such as NaH in a solvent such as DMF at or around ambient temperature to provide alkoxy compound CXXVIII (Scheme 19).

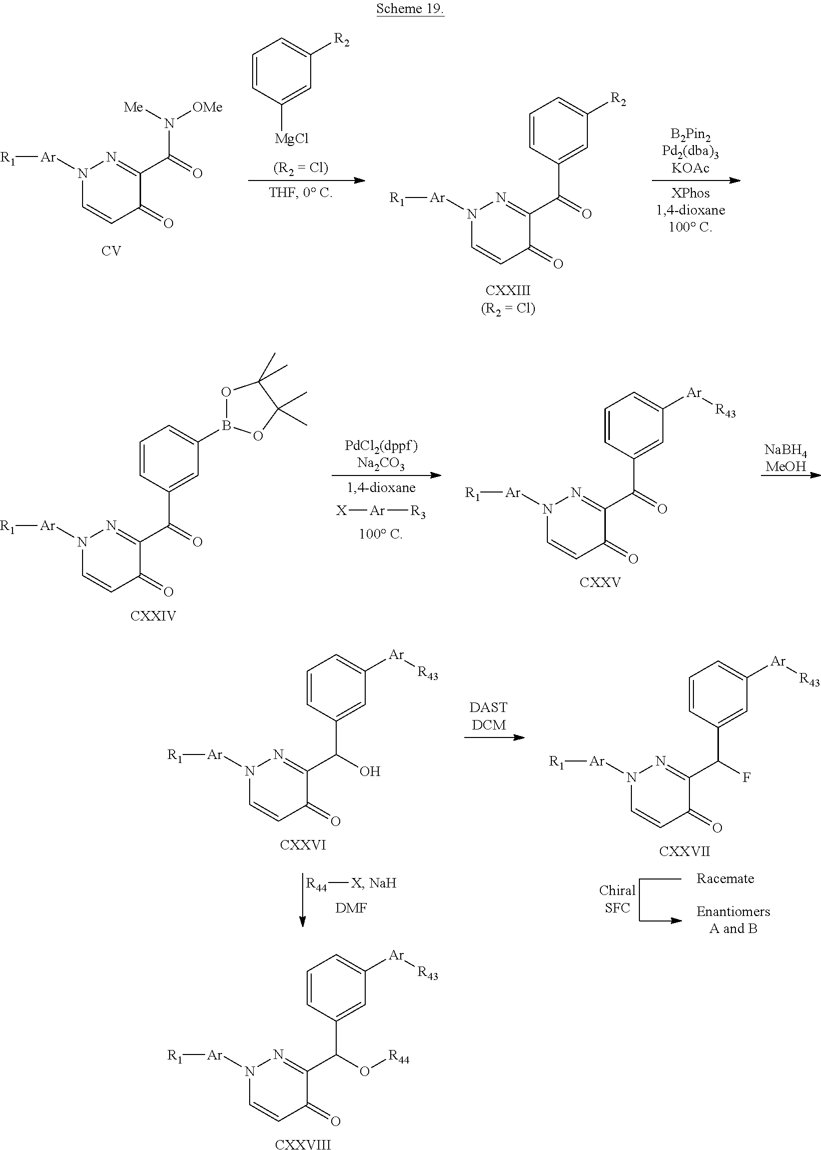

Unsubstituted pyrazole VII′ is reacted with an appropriately substituted alcohol under Mitsunobu conditions (e.g., DEAD, PPh₃) in a suitable solvent such as THF at or around ambient temperature to alkylated pyrazole CXXIX.

Alternatively, unsubstituted pyrazole VIP is reacted with an appropriately substituted alkyl halide in the presence of a suitable base such as Cs₂CO₃ in a solvent such as DMF at or around 150° C. under microwave irradiation to provide N-substituted product CXXIX, with an additional transformation to remove a protecting group (such as Boc) performed as required, or C-substituted product CXXX.

Alternatively, unsubstituted-pyrazole VIP is reacted with an appropriately substituted epoxide in the presence of a suitable base such as Cs₂CO₃ in a solvent such as DMF at or around 150° C. under microwave irradiation to afford alcohol CXXXI (Scheme 20).

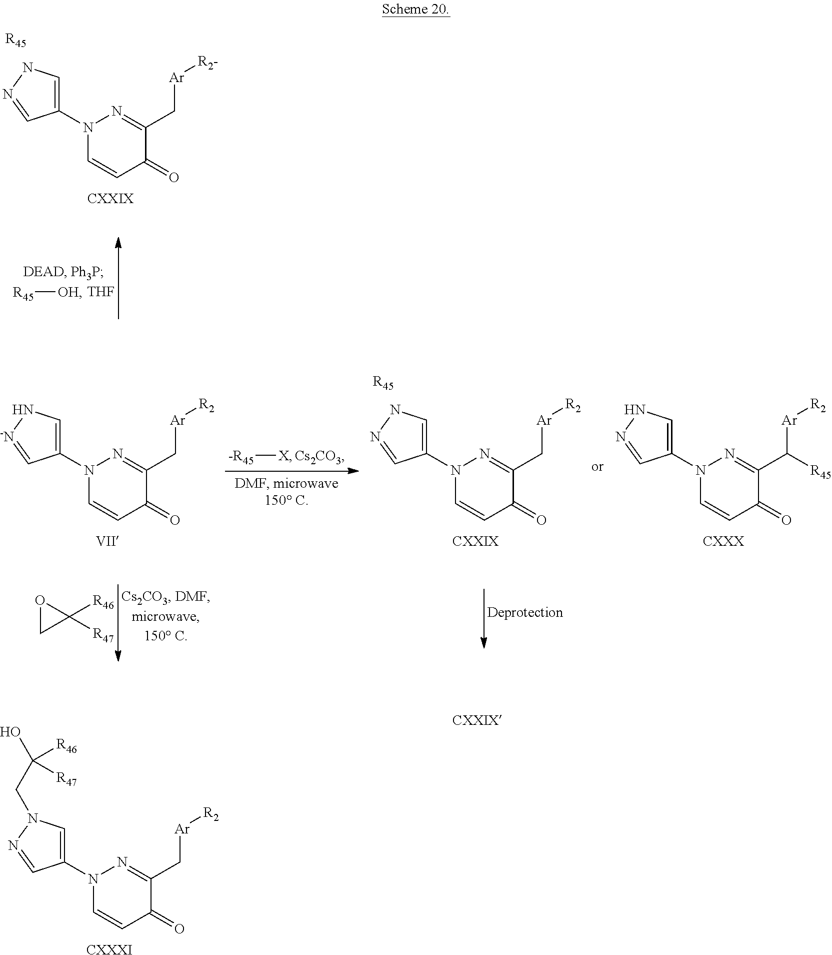

For a specific example wherein CXXIX′ contains an unsubstituted azetidine, N-methylation can be achieved by treating with iodomethane in the presence of a suitable base such as Cs₂CO₃ in a solvent such as DMF at or around ambient temperature to provide CXXXII (Scheme 21).

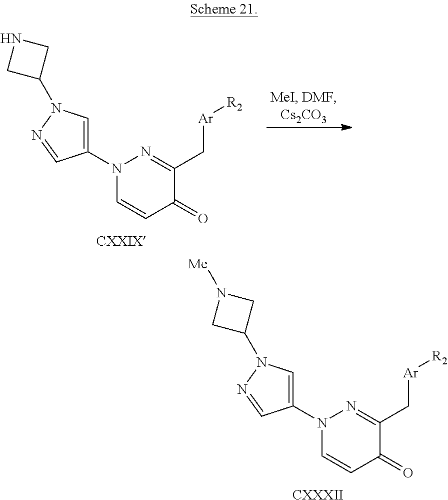

Weinreb amide CV is treated with [3-(benzyloxy)phenyl]magnesium bromide in a solvent such as THF at or around −78° C. to afford the corresponding ketone CXXXIII. Reduction of the ketone using a reducing agent such as sodium borohydride in a solvent such as MeOH at or around ambient temperature affords alcohol CXXXIV. Treatment of CXXXIV under hydrogenolysis conditions using hydrogen at balloon pressure or above and a suitable palladium catalyst such as 10 wt % Pd/C in a solvent such as MeOH at or around ambient temperature affords phenol CXXXV. Treatment of CXXXV with 1,1,1-trifluoro-N-phenyl-N-[(trifluoromethyl)sulfonyl]methanesulfonamide in the presence of a suitable base such as DIPEA in a solvent such as THF at or around ambient temperature affords aryl triflate intermediate CXXXVI. Triflate CXXXVI is then reacted with an appropriately substituted aryl stannane under palladium catalyzed cross-coupling conditions using a catalyst/ligand system such as Pd₂(dba)₃/X-Phos in the presence of additives such as CuI, CsF and LiCl in a solvent such as DMF at or around 100° C. to afford biaryl CXXXVII. Alternatively, aryl triflate CXXXVI is converted to the corresponding boronic ester C-XXVIII in a similar manner as described above (for boronic ester synthesis Method B) using a palladium/XPhos catalyst system. Boronic ester CXXXVIII is then reacted with an appropriately substituted aryl halide under palladium catalyzed cross-coupling conditions using a catalyst/ligand system such as Pd₂(dba)₃/XPhos in the presence of a suitable base such as Cs₂CO₃ in a solvent such as 1,4-dioxane at or around 100° C. to afford biaryl CXXXVII.

Alternatively, aryl chloride VI (R₂═Cl) is converted to the corresponding boronic ester CXXXVIII in a similar manner as described above (for boronic ester synthesis Method B) using a palladium/XPhos catalyst system. Reacting intermediate CXXXVII in the manner described above affords biaryl CXXXVII (Scheme 22).

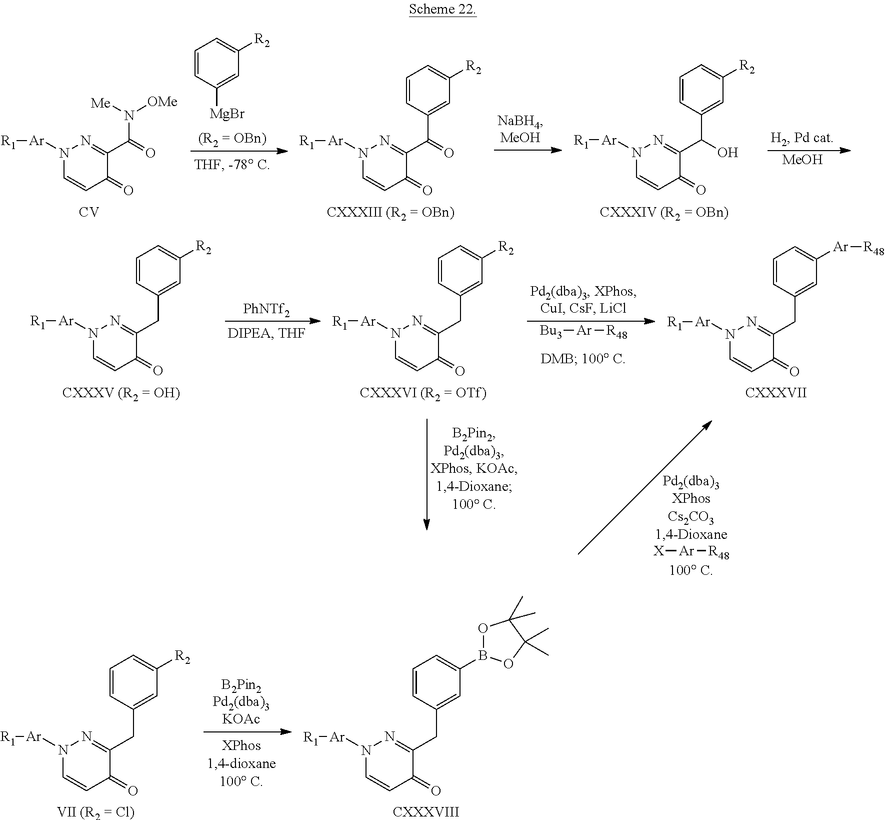

Aryl chloride LXXIX (R₂═Cl, racemate or single enantiomer) is converted to the corresponding boronic ester CXXXIX in a similar manner as described above (for boronic ester synthesis Method B) using a palladium/XPhos catalyst system. Boronic ester CXXXIX is then reacted with an appropriately substituted aryl halide under palladium catalyzed cross-coupling conditions using a catalyst/ligand system such as Pd₂(dba)₃/XPhos in the presence of a suitable base such as Cs₂CO₃ in a solvent such as 1,4-dioxane at or around 100° C. to afford biaryl CXL. For cases wherein racemic CXXXIX was used, separation of enantiomers A and B can be achieved using preparative chiral supercritical fluid chromatography. An additional transformation to remove a protecting group (such as Boc) may be performed as required (Scheme 23).

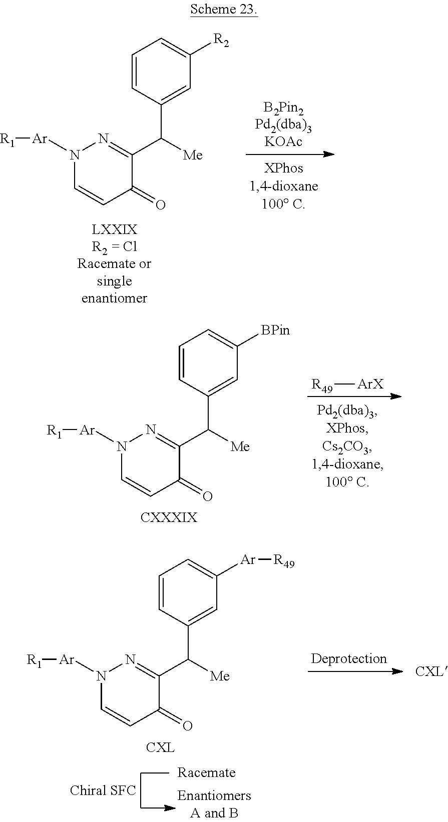

Carboxylic acid VII is treated with an appropriately substituted amine using a suitable amide coupling reagent such as TBTU in a solvent such as CHCl₃ at or around ambient temperature to afford amide CXLI (Scheme 24).

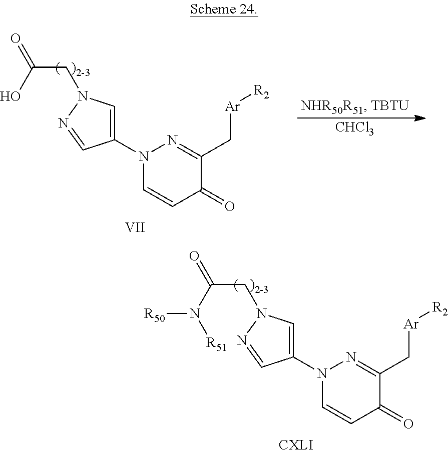

Unsubstituted 1,2,4-triazole VII′ is reacted with an appropriately substituted alkyl halide in the presence of a suitable base such as Cs₂CO₃ in a solvent such as DMF at or around 150° C. under microwave irradiation to provide alkyl triazole CXLII (Scheme 25).

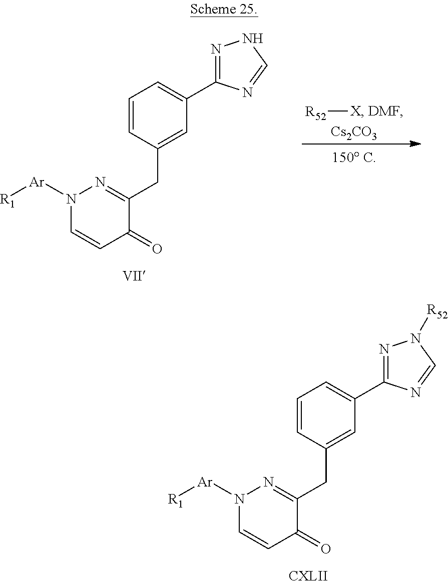

Aryl bromide VII (R₁═bromo) is treated with Zn(CN)₂ in the presence of an appropriate palladium catalyst (e.g., Pd(PPh₃)₄) in a suitable solvent such as DMF under microwave heating (at or around 120° C.) to afford the corresponding aryl nitrile CXLII.

Alternatively, aryl bromide VII (R₁=bromo) is reacted with an appropriately substituted boronic acid or ester using a suitable palladium catalyst such as Pd(PPh₃)₄ and base such as Na₂CO₃ in a solvent system such as DME/water under microwave irradiation (at or around 100° C.) to afford CXLIV.

Alternatively, aryl bromide VII (R₁=bromo) is reacted with Mo(CO)₆ and a substituted amine using an appropriate palladium catalyst (e.g., Hermann's catalyst, P^{t}Bu₃.HBF₄) in the presence of a suitable base such as DBU in a solvent such as 1,4-dioxane under microwave heating (at or around 140° C.) to afford amide CXLV or des-bromo CXLVI. Alternatively, aryl bromide VII (R₁=bromo) is reacted with CO and a substituted amine using an appropriate palladium catalyst system such as Pd(OAc)₂/dppp in a solvent such as DMF at or around 70° C. to afford amide CXLV (Scheme 26).

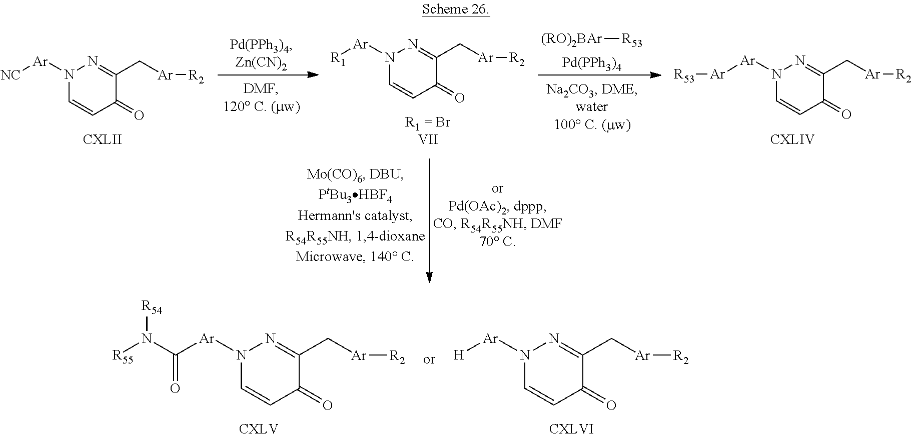

Aryl nitrile VII (R₁═CN, R₃═H) or LXXIX (R₁═CN, R₃=Me) is reacted with hydrogen peroxide in the presence of a suitable base such as K₂CO₃ in a solvent such as DMSO at or around 0° C. to ambient temperature to give primary amide CXLVII (Scheme 27).

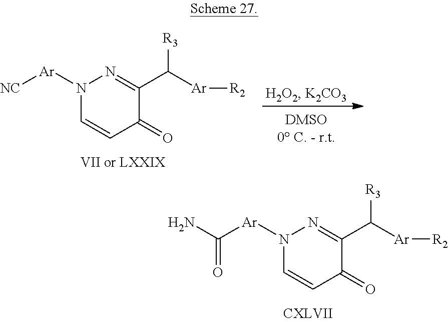

Aryl nitrile VII (R₂═CN) is reacted with hydroxylamine using an appropriate solvent system such as MeOH/EtOH at or around 80° C. to afford intermediate CXLVIII. Intermediate CXLVIII is then treated with an appropriately substituted carboxylic acid in the presence of a coupling reagent such as EDC in a solvent such as DMF at or around 50° C. Increasing the temperature to approximately 100° C. furnishes oxadiazole CXLIX (Scheme 28).

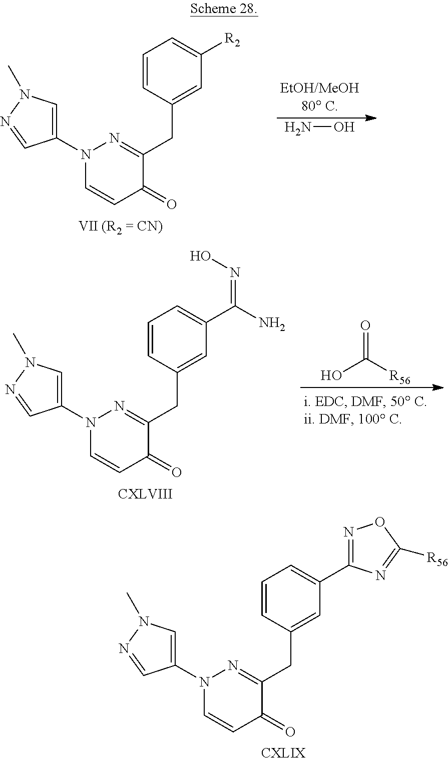

For a specific example wherein VII is 3-{3-[5-(1,4-dioxaspiro[4.5]dec-8-yloxy)pyrimidin-2-yl]benzyl}-1-(1-methyl-1H-pyrazol-4-yl)pyridazin-4(1H)-one, treatment with a suitable acid such as 6 NHCL in a solvent such as THF at or around ambient temperature gives cyclohexanone CL. Treatment of ketone CL with a suitable hydride source such as NaBH₄ in a solvent such as MeOH at or around 0° C. to ambient temperature gives cyclohexanol CLI. The cis/trans mixture can be purified by reverse phase preparative HPLC to give the cis and trans isomers (Scheme 29).

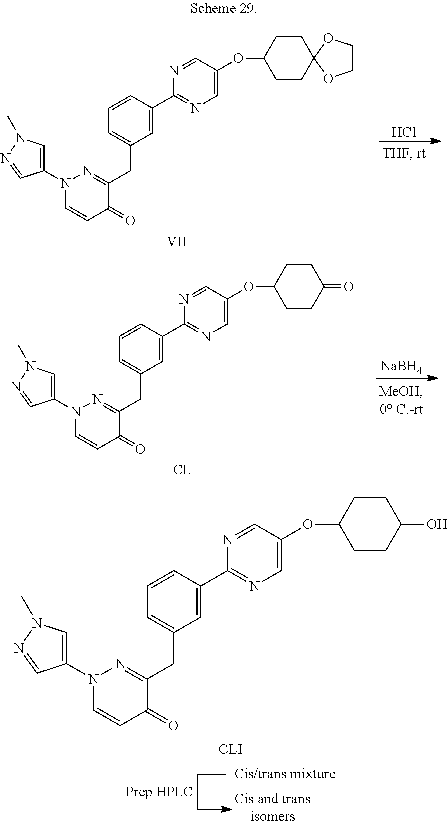

For a specific example wherein VII is tert-butyl 4-[2-(3-{[1-(1-methyl-1H-pyrazol-4-yl)-4-oxo-1,4-dihydropyridazin-3-yl]methyl}phenyl)pyrimidin-5-yl]-3,6-dihydropyridine-1(2H)-carboxylate, treatment with a suitable dihydroxylation catalyst system such as OsO₄/NMO in a solvent system such as THF/water at ambient temperature affords diol CLII. Diol CLII is then reacted with a suitable fluorinating reagent such as DAST in a solvent such as DCM at or around 0° C. to afford fluorohydrin CLIII. Subsequent removal of the Boc group using an appropriate acid source (e.g., HCl/dioxane) in a solvent such as MeOH at ambient temperature furnishes the HCl salt of amine CLIV (Scheme 30).

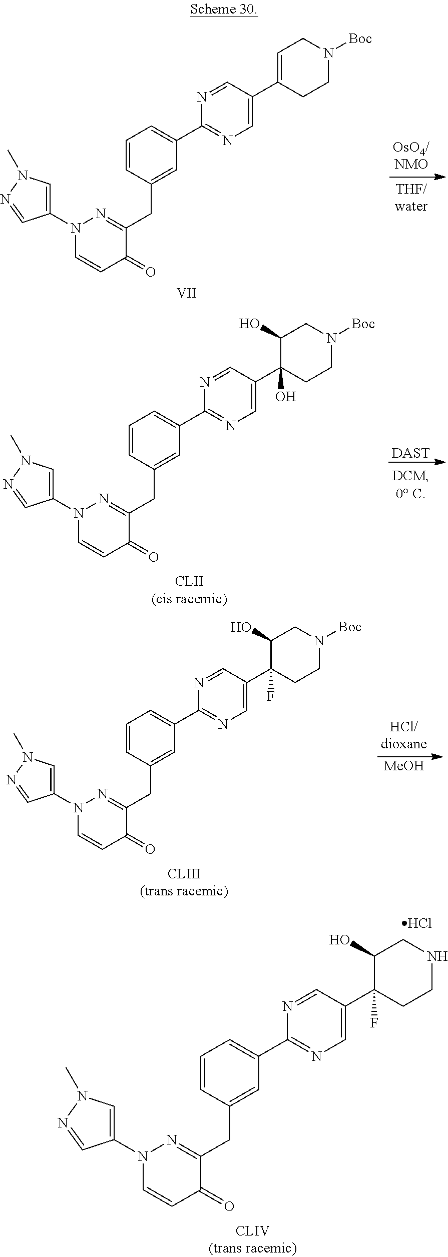

For a specific example wherein VII is 3-{3-[5-(2-methoxyethoxy)pyrimidin-2-yl]benzyl}-1-(1-methyl-1H-pyrazol-4-yl)pyridazin-4(1)-one, treatment with an iodinating reagent such as bis(pyridine)iodonium tetrafluoroborate in the presence of a suitable acid such as triflic acid in a solvent such as DCM at or around ambient temperature provides iodo pyridazinone CLV. Coupling of iodide CLV with potassium methyltrifluoroborate in the presence of a suitable catalyst such as PdCl₂(dppf).DCM and base such as Cs₂CO₃ in a solvent such as THF/H₂O at or around 100° C. provides methyl pyridazinone CLVI (Scheme 31).

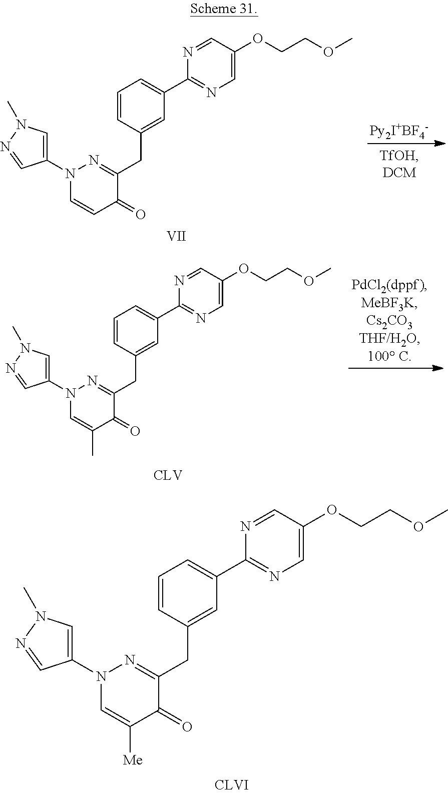

Treatment of VII (wherein Ar—R₁ is 1-methylpyrazole and R₂ is an alkoxy pyrimidine) with Selectfluor in a solvent such as MeCN at or around 150° C. under microwave irradiation provides difluoro product CLVII (Scheme 32).

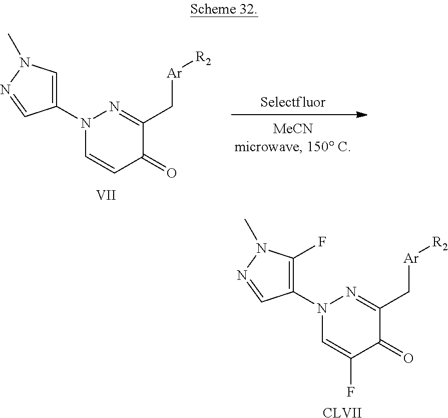

Reaction of aryl carboxylic acid CLVIII with N-methoxymethanamine hydrochloride under amide coupling conditions such as EDC/HOBt using a base such as DIPEA in a solvent such as DMF at or around ambient temperature affords Weinreb amide intermediate CLIX. Treatment of CLIX with methylmagnesium bromide in a solvent such as THF at or around −78° C. gives methyl ketone CLX. Condensation of CLX with EtOAc using an appropriate base such as LiHMDS in a solvent such as THF affords diketone CLXI. Diketone CLXI is then reacted with an aryl diazonium chloride solution (generated by the treatment of an arylamine I with sodium nitrite in aqueous HCl at or around 5° C.) in the presence of a suitable base such as NaOAc in a solvent such as EtO/H₂O to give diazene CLXII. CLXII is heated in DMFDMA as solvent to afford cyclized pyridazinone CLXIII. Treatment of ketone CLXIII with a suitable hydride source such as NaBH₄ in a solvent such as MeOH at or around ambient temperature provides alcohol CLXIV. Treatment of alcohol CLXIV with a fluorinating reagent such as DAST in a solvent such as DCM provides fluoro product CLXV Scheme 33).

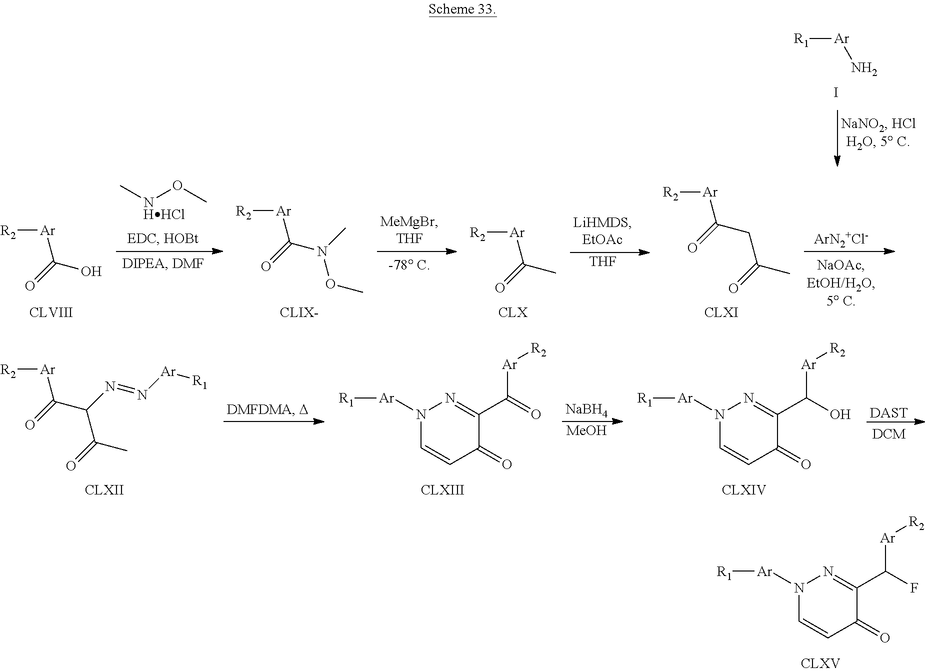

Weinreb amide intermediate CV is reacted with an appropriately substituted aryl Grignard reagent (i.e., [4-(tetrahydro-2H-pyran-2-yloxy)phenyl]magnesium bromide) in a suitable solvent such as THF at or around −78° C. to afford ketone intermediate CLXVI. The THP group of CLXVI is removed under acidic conditions (such as HCl in 1,4-dioxane at or around ambient temperature) to afford phenol CLXVII. Phenol intermediate CLXVII is treated with Et₃SiH in the presence of HF-pyridine in a solvent system such as DCM/TFA at or around-60° C. to afford CLXVIII (Scheme 34).

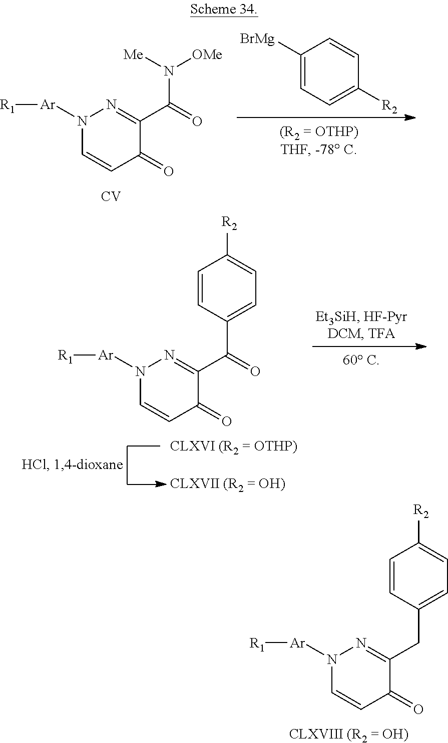

Pyridazinone carboxylic acid IV is treated with DPPA in the presence of a suitable base such as Et₃N in tert-BuOH to give Boc-protected amine CLXIX. Removal of the Boc group under acidic conditions (such as HCl in 1,4-dioxane) affords amino pyridazinone CLXX. Treatment of CLXX with sodium nitrite in concentrated HCl at or around 0° C. provides chloro pyridazinone intermediate CLXXI. Reaction of CLXXI with a phenol (generated by treating boronic ester LXI with hydrogen peroxide and sodium hydroxide in THF) in the presence of a suitable base such as K₂CO₃ in a solvent such as DMF at or around 100° C. gives ether product CLXXII (Scheme 35).

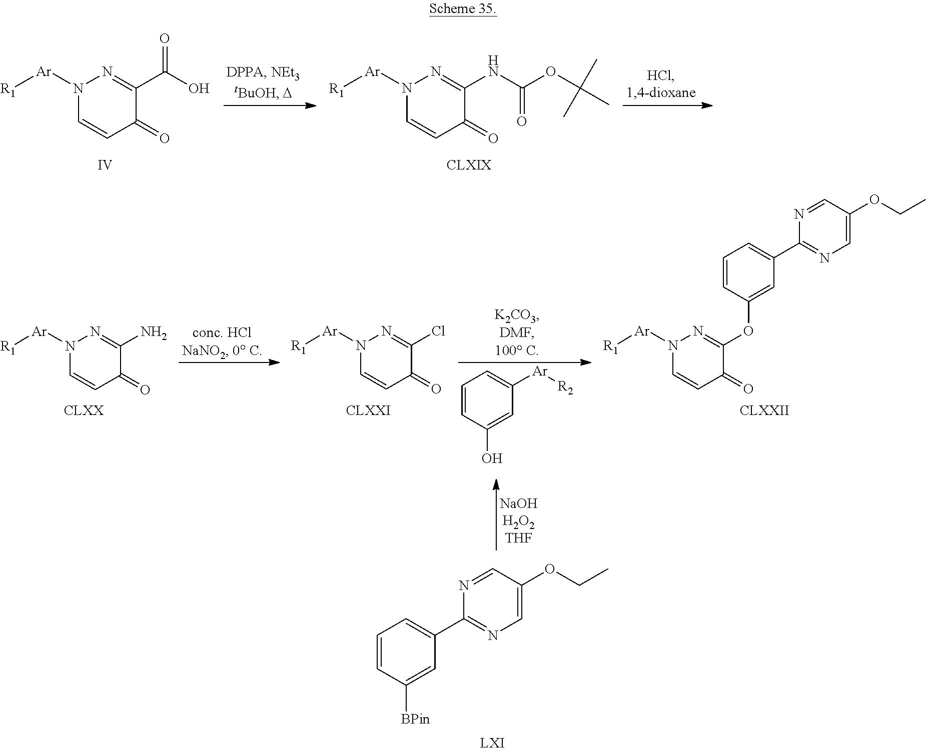

Biaryl chloride LX is hydroxylated with KOH in the presence of a suitable catalyst system such as Pd₂(dba)₃/Me₄^{t}BuXPhos in a solvent such as 1,4-dioxane/H₂O at or around 100° C. Treatment of the product phenol in situ with dimethylcarbamothioic chloride provides thiocarbamate CLXXIII. Rearrangement of the thiocarbamate at or around 250° C. under microwave irradiation in a solvent such as NMP followed by hydrolysis with a base such as aqueous NaOH at or around 100° C. provides a solution of the corresponding thiophenol. Addition of chloro pyridazinone CLXXI and continued heating at or around 50° C. provides thioether CLXXIV (Scheme 36).

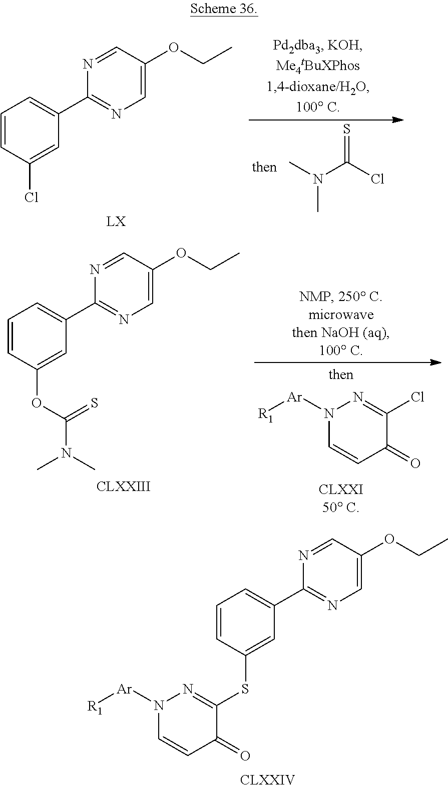

The invention will now be illustrated in the following non-limiting Examples in which, unless otherwise stated:
All the end products of the formula I were analyzed by NMR, LCMS.
Intermediates were analyzed by NMR and/or TLC and/or LCMS.
Most compounds were purified by flash chromatography on silica gel (including MPLC), reverse phase preparative HPLC, recrystallization and/or swish (suspension in a solvent followed by filtration of the solid). Mixtures of stereoisomers were separated by chiral HPLC or chiral SFC. The course of the reactions were followed by thin layer chromatography (TLC) and/or LCMS and reaction times are given for illustration only.

### Scheme 1

### Intermediate #1

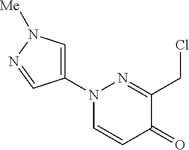

### 3-(Chloromethyl)-1-(1-methyl-1H-pyrazol-4-yl)pyridazin-4(1H)-one

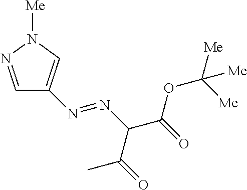

### Step 1. tert-Butyl 2=[(1-methyl-1H-pyrazol-4-yl)diazenyl]-3-oxobutanoate

1-Methyl-1H-pyrazol-4-amine (17.0 g, 175 mmol) was dissolved in concentrated HCl (50 mL)/water (260 mL) and cooled to 0° C. A solution of sodium nitrite (12.7 g, 184 mmol) in water (180 mL) was added dropwise while maintaining the internal temperature at <4° C. On complete addition, the mixture was stirred at 0° C. for 20 minutes. The resulting diazonium chloride solution was added dropwise to a solution of tert-butyl acetoacetate (29.0 mL, 175 mmol) and sodium acetate (1-87g, 2280 mmol) in water (220 mL)/ethanol (220 mL) at 0° C. The resulting mixture was stirred at 0° C. for 15 minutes. Saturated NaHCO₃ was added and the products extracted into EtOAc (3×). The combined organic extracts were dried over Na₂SO₄ and concentrated in vacuo to give tert-butyl 2-[(1-methyl-1H-pyrazol-4-yl)diazenyl]-3-oxobutanoate as a red oil.

LRMS (ESI) calc'd for C12H19N4O3 [M+H]⁺: 267. Found: 267.

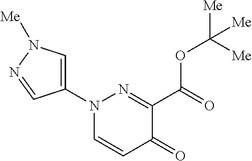

### Step 2. tert-Butyl 1-(1-methyl-1H-pyrazol-4-yl)-4-oxo-1,4-dihydropyridazine-3-carboxylate

tert-Butyl 2-[(1-methyl-1H-pyrazol-4-yl)diazenyl]-3-oxobutanoate (47.0 g, 176 mmol) was stirred in refluxing DMFDMA (350 mL) for 1 hour. Room temperature was attained before cooling the reaction mixture in the freezer overnight. The solvent was decanted off, Et₂O was added and the red solid collected by filtration and washed with Et₂O followed by water to give tert-butyl 1-(1-methyl-1H-pyrazol-4-yl)-4-oxo-1,4-dihydropyridazine-3-carboxylate as a pink solid.

LRMS (ESI) calc'd for C13H17N4O3 [M+H]⁺: 277. Found: 277.

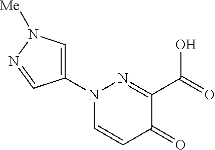

### Step 3. 1-(1-Methyl-1H-pyrazol-4-yl)-4-oxo-1,4-dihydropyridazine-3-carboxylic acid

tert-Butyl 1-(1-methyl-1H-pyrazol-4-yl)-4-oxo-1,4-dihydropyridazine-3-carboxylate (35.1 g, 127 mmol) was stirred in DCM (580 mL)/TFA (58 mL) at r.t. for 2 hours. The solvent was removed in vacuo and the residue triturated in Et₂O to give 1-(1-methyl-1H-pyrazol-4-yl)-4-oxo-1,4-dihydropyridazine-3-carboxylic acid as a pink solid.

LRMS (ESI) calc'd for C9H9N4O3 [M+H]⁺: 221. Found: 221.

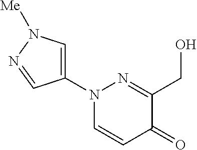

### Step 4. 3-Hydroxymethyl)-1-methyl-1H-pyrazol-4-yl)pyridazin-4(1H)-one

1-(1-Methyl-1H-pyrazol-4-yl)-4-oxo-1,4-dihydropyridazine-3-carboxylic acid (27.4 g, 125 mmol) was taken up in THF (1250 mL) and cooled to 0° C. Isobutyl chloroformate (19.6 mL, 1.49 mmol) was added, followed by N-methylmorpholine (16.4 mL, 149 mmol) and the resulting mixture stirred at 0° C. for 1 hour. A solution of sodium borohydride (14.1 g, 374 mmol) in water (75 mL) was prepared and immediately added to the reaction mixture at such a rate so as to avoid bubbling over. After 1 hour at 0° C., additional water was added and the solvent removed in vacuo while loading onto silica. Purification of the residue by flash chromatography (MPLC, 0-10% MeOH-DCM) gave 3-(hydroxymethyl)-1-(1-methyl-1H-pyrazol-4-yl)pyridazin-4(1H)-one as a yellow solid.

LRMS (ESI)-calc'd for C9H11N4O2 [M+H]⁺: 207. Found: 207.

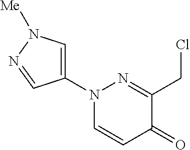

### Step 5. 3-(Chloromethyl)-1-(1-methyl-1H-pyrazol-4-yl)pyridazin-4(1H)-one

3-(Hydroxymethyl)-1-(1-methyl-1H-pyrazol-4-yl)pyridazin-4(1H)-one (20.2 g, 98.0 mmol) was taken up in MeCN (980 mL). Thionyl chloride (35.7 mL, 489 mmol) was added dropwise and the resulting mixture stirred at r.t. for 3 hours. The reaction mixture was dry loaded onto silica and the residue purified by flash chromatography (MPLC, 0-10% MeOH-DCM). The isolated product was taken up in 10% MeOH-DCM and washed with saturated NaHCO₃. The organic phase was dried over MgSO₄, filtered and concentrated in vacuo. The residue was triturated in Et₂O to give 3-(chloromethyl)-1-(1-methyl-1H-pyrazol-4-yl)pyridazin-4(1H)-one as a beige solid.

LRMS (ESI) calc'd for C9H10ClN4O [M+H]⁺: 225. Found: 225.

The following intermediates were prepared according to Scheme 1-following similar procedures described for Intermediate #1, which can be achieved by those of ordinary skill in the art of organic synthesis.

| | | | Exact Mass |
|---|---|---|---|
| Intermediate | Structure | IUPAC Name | [M + H]⁺ |
| 2 | 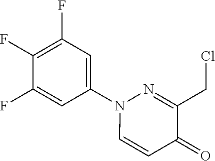 | 3-(chloromethyl)-1-(3,4,5- trifluorophenyl)pyridazin- 4(1H)-one | Calc'd 275, found 275 |
| 3 | 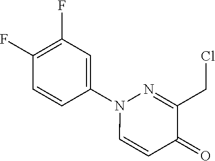 | 3-(chloromethyl)-1-(3,4- difluorophenyl)pyridazin- 4(1H)-one | Calc'd 257, found 257 |
| 4 | 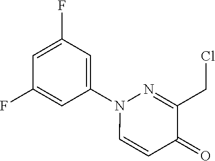 | 3-(chloromethyl)-1-(3,5- difluorophenyl)pyridazin- 4(1H)-one | Calc'd 257, found 257 |
| 5 | 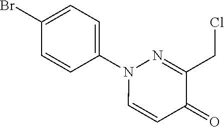 | 1-(4-bromophenyl)-3- (chloromethyl)pyridazin- 4(1H)-one | Calc'd 300, found 300 |
| 6 | 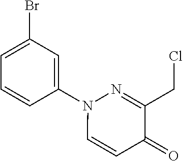 | 1-(3-bromophenyl)-3- (chloromethyl)pyridazin- 4(1H)-one | Calc'd 300, found 300 |
| 7 | 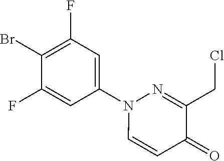 | 1-(4-bromo-3,5- difluorophenyl)-3- (chloromethyl)pyridazin- 4(1H)-one | Calc'd 336, found 336 |
| 8 | 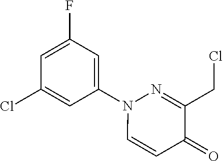 | 1-(3-chloro-5- fluorophenyl)-3- (chloromethyl)pyridazin- 4(1H)-one | Calc'd 273, found 273 |
| 9 | 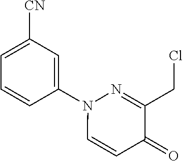 | 3-[3-(chloromethyl)-4- oxopyridazin-1(4H)- yl]benzonitrile | Calc'd 246, found 246 |
| 10 | 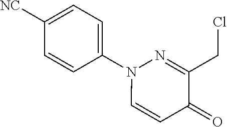 | 4-[3-(chloromethyl)-4- oxopyridazin-1(4H)- yl]benzonitrile | Calc'd 246, found 246 |
| 11 | 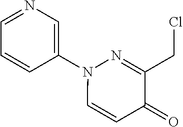 | 3-(chloromethyl)-1- (pyridin-3-yl)pyridazin- 4(1H)-one | Calc'd 222, Found 222 |
| 12 | 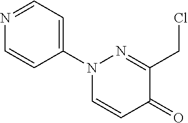 | 3-(chloromethyl)-1- (pyridin-4-yl)pyridazin- 4(1H)-one | Calc'd 222, Found 222 |
| 13 | 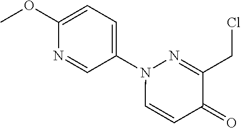 | 3-(chloromethyl)-1-(6- methoxypyridin-3- yl)pyridazin-4(1H)-one | Calc'd 252, Found 252 |
| 14 | 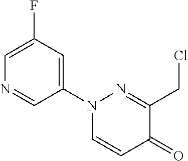 | 3-(chloromethyl)-1-(5- fluoropyridin-3- yl)pyridazin-4(1H)-one | Calc'd 240, Found 240 |
| 15 | 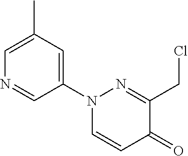 | 3-(chloromethyl)-1-(5- methylpyridin-3- yl)pyridazin-4(1H)-one | Calc'd 235, Found 235 |
| 16 | 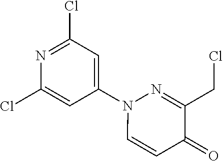 | 3-(chloromethyl)-1-(2,6- dichloropyridin-4- yl)pyridazin-4(1H)-one | Calc'd 290, Found 290 |
| 17 | 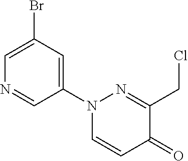 | 1-(5-bromopyridin-3-yl)- 3- (chloromethyl)pyridazin- 4(1H)-one | Calc'd 300, Found 300 |
| 18 | 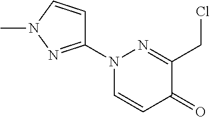 | 3-(chloromethyl)-1-(1- methyl-1H-pyrazol-3- yl)pyridazin-4(1H)-one | Calc'd 225, Found 225 |
| 19 | 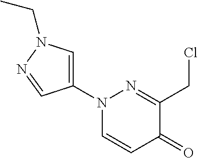 | 3-(chloromethyl)-1-(1- ethyl-1H-pyrazol-4- yl)pyridazin-4(1H)-one | Calc'd 239, Found 239 |
| 20 | 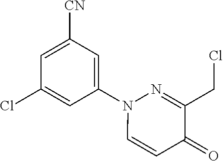 | 3-chloro-5-[3- (chloromethyl)-4- oxopyridazin-1(4H)- yl]benzonitrile | Calc'd 280, Found 280 |
| 21 | 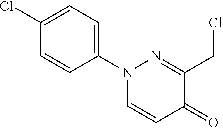 | 3-(chloromethyl)-1-(4- chlorophenyl)pyridazin- 4(1H)-one | Calc'd 255, Found 255 |
| 22 | 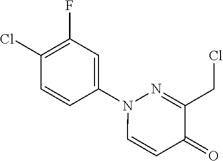 | 1-(4-chloro-3- fluorophenyl)-3- (chloromethyl)pyridazin- 4(1H)-one | Calc'd 273, Found 273 |
| 23 | 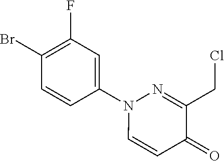 | 1-(4-bromo-3- fluorophenyl)-3- (chloromethyl)pyridazin- 4(1H)-one | Calc'd 319, Found 319 |

### Scheme 1

### Intermediate #24

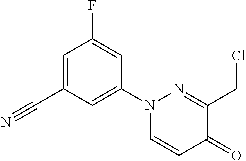

### 3-[3-(Chloromethyl)-4-oxopyridazin-1(4H)-yl]-5-fluorobenzonitrile

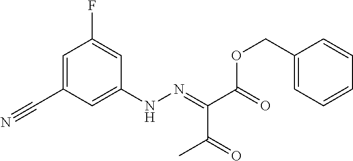

### Step 1. Benzyl (2E)-2-[2-(3-cyano-5-fluorophenyl)hydrazinylidene]-3-oxobutanoate

3-Amino-5-fluorobenzonitrile (4.93 g, 36.2 mmol) was dissolved in concentrated HCl (11 mL) and water (50 mL) and cooled to 0° C. A solution of sodium nitrite (2.66 g, 38.6 mmol) in water (37 mL) was added dropwise at such a rate to maintain the internal temperature below 4° C. (additional ice was added directly to the reaction mixture to facilitate cooling). On complete addition, the reaction mixture was stirred at 0° C. for 75 minutes. Meanwhile, a separate flask was charged with ethanol (42 mL), saturated aqueous sodium acetate solution (42 mL) and benzyl acetoacetate (6.5 mL, 38 mmol) and the mixture was cooled to 0° C. The diazonium chloride solution was slowly added to the benzyl acetoacetate solution, rinsing with EtOH, and the mixture stirred at 0° C. for 1 hr. The resulting precipitate was collected by filtration and dried to obtain an orange solid that was purified by flash chromatography (MPLC, 0-100% EtOAc-hexanes) to obtain benzyl (2E)-2-[2-(3-cyano-5-fluorophenyl)hydrazinylidene]-3-oxobutanoate.

LRMS (ESI) calc'd for C18H15FN3O3 [M+H]⁺: 340. Found: 340.

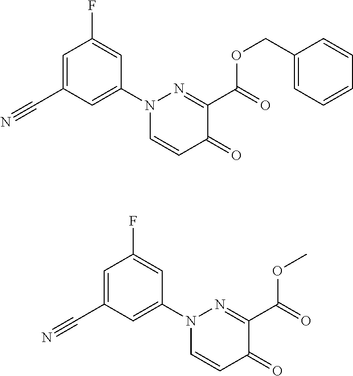

### Step 2. Benzyl 1-(3-cyano-5-fluorophenyl)-4-oxo-1,4-dihydropyridazine-3-carboxylate and methyl 1-(3-cyano-5-fluorophenyl-4-oxo-1,4-dihydropyridazine-3-carboxylate (mixture)

Benzyl (2E)-2-[2-(3-cyano-5-fluorophenyl)hydrazinylidene]-3-oxobutanoate (7.31 g, 21.5 mmol) was taken up in DMFDMA (75 mL) and heated to 75° C. for 5 hr. The mixture was cooled to r.t. and poured into water. The precipitate was collected by filtration, taken up in DCM, dried over-magnesium sulfate, filtered and concentrated in vacuo. Purification of the residue by flash chromatography (MPLC, 0-20% MeOH-DCM) gave a 3:5 mixture of benzyl 1-(3-cyano-5-fluorophenyl)-4-oxo-1,4-dihydropyridazine-3-carboxylate and methyl 1-(3-cyano-5-fluorophenyl)-4-oxo-1,4-dihydropyridazine-3-carboxylate.

LRMS (ESI) calc'd for C19H13FN3O3 [M+H]⁺: 350. Found: 350.

LRMS (ESI) calc'd for C13H8FN3O3 [M+H]⁺: 274. Found: 274.

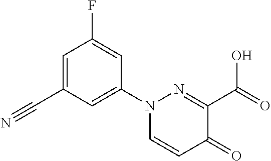

### Step 3. 1-(3-Cyano-5-fluorophenyl)-4-oxo-1,4-dihydropyridazine-3-carboxylic acid

A pressure flask was charged with the 3:5 mixture of benzyl 1-(3-cyano-5-fluorophenyl)-4-oxo-1,4-dihydropyridazine-3-carboxylate and methyl 1-(3-cyano-5-fluorophenyl)-4-oxo-1,4-dihydropyridazine-3-carboxylate (2.71 g, 8.99 mmol), sodium iodide (2.0 g, 13 mmol) and pyridine (15 mL). The flask was sealed and the reaction heated to 100° C. for 20 hours. The reaction mixture was cooled to r.t. and filtered. The precipitate was washed with hexanes before suspending in 1 N HCl. After stirring, the solid was collected by filtration, washed with water and dried to obtain 1-(3-cyano-5-fluorophenyl)-4-oxo-1,4-dihydropyridazine-3-carboxylic acid.

LRMS (ESI) calc'd for C12H7FN3O [M+H]⁺: 260. Found: 260.

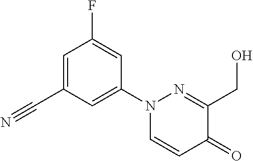

### Step 4. 3-Fluoro-5-[3-(hydroxymethyl)-4-oxopyridazin-1(4H)-yl]benzonitrile

An oven-dried, nitrogen-cooled 250 mL round bottom flask was charged with 1-(3-cyano-5-fluorophenyl)-4-oxo-1,4-dihydropyridazine-3-carboxylic acid-(1.67 g, 6.44 mmol), sealed under a nitrogen atmosphere and THF (50 mL). The mixture was cooled to 0° C. and isobutyl chloroformate (0.93 mL, 7.1 mmol) was added, followed by dropwise addition of N-methylmorpholine (0.85 mL, 7.7 mmol). On complete addition, the reaction mixture was stirred at 0° C. for 1 hour. A freshly prepared solution of sodium borohydride (0.74 g, 20 mmol) in water (5 mL) (the septum was removed prior to addition to prevent gas buildup) was added dropwise and the resulting mixture stirred at r.t. for 1 hour. Water was added and the THF removed in vacuo, at which point a precipitate formed. This precipitate was collected by filtration, washed with water and dried to obtain 3-fluoro-5-[3-(hydroxymethyl)-4-oxopyridazin-1(4H)-yl]benzonitrile.

LRMS (ESI) calc'd for C12H9FN3O2 [M+H]⁺: 246. Found: 246.

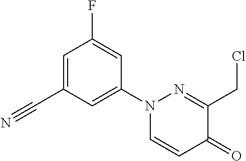

### Step 5. 3-[3-(Chloromethyl)-4-oxopyridazin-1(4H)-yl]-5-fluorobenzonitrile

Thionyl chloride (1.0 mL, 14 mmol) was added to a stirring mixture of 3-fluoro-5-[3-(hydroxymethyl)-4-oxopyridazin-1(4H)-yl]benzonitrile (0.81 g, 3.3 mmol) in MeCN (15 mL). The resulting mixture was stirred at r.t. for 2.5 hours before concentrating in vacuo. Ethyl acetate was added and the solution washed with saturated NaHCO₃ (2×) and brine, dried over MgSO₄, filtered and concentrated in vacuo to obtain 3-[3-(chloromethyl)-4-oxopyridazin-1(4H)-yl]-5-fluorobenzonitrile.

LRMS (ESI) calc'd for Cl2H8ClFN3O [M+H]⁺: 264. Found: 264.

### Scheme 2

### Intermediate #25

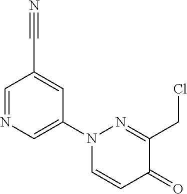

### 5-[3-(Chloromethyl)-4-oxopyridazin-1(4H)-yl]pyridine-3-carbonitrile

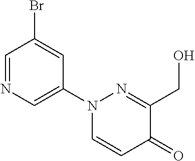

### Steps 1-4. 1-(5-Bromopyridin-3-yl)-3-(hydroxymethyl)pyridazin-4(1H)-one

1-(5-Bromopyridin-3-yl)-3-(hydroxymethyl)pyridazin-4(1H)-one was prepared from 5-bromopyridin-3-amine according to the procedures described for 3-(hydroxymethyl)-1-(1-methyl-1H-pyrazol-4-yl)pyridazin-4(1H)-one (Scheme 1, Intermediate #1 Steps 1-4).

LRMS (ESI) calc'd for Cl0H9BrN3O2 [M+H]⁺: 282. Found: 282.

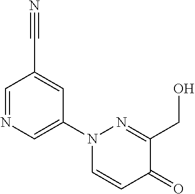

### Step 5. 5-[3-(Hydroxymethyl)-4-oxopyridazin-1 (4H)-yl]pyridine-3-carbonitrile

A 20 mL microwave vial was charged with 1-(5-bromopyridin-3-yl)-3-(hydroxymethyl)pyridazin-4(1H)-one (0.97 g, 3.4 mmol), tetrakis(triphenylphosphine) palladium (0) (0.40 g, 0.35 mmol) and zinc cyanide (0.50 g, 4.26 mmol) at which point DMF (10 mL) was added and the reaction mixture was degassed with a stream of nitrogen gas. The vial was capped with a septa and heated to 12° C. for 30 min under microwave irradiation. The reaction mixture was filtered through celite and eluted with EtOAc, then the filtrate was concentrated in vacuo and purified flash chromatography (MPLC, 0-20% MeOH/DCM) to obtain 5-[3-(hydroxymethyl)-4-oxopyridazin-1 (4H)-yl]pyridine-3-carbonitrile.

LRMS (ESI) calc'd for C11H8N4O2 [M+H]⁺: 229. Found: 229.

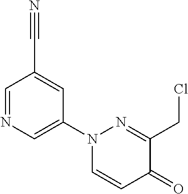

### Step 6. 5-[3-(Chloromethyl)-4-oxopyridazin-1(4H)-yl]pyridine-3-carbonitrile

To a stirring mixture of 5-[3-(hydroxymethyl)-4-oxopyridazin-1(4H)-yl]pyridine-3-carbonitrile (468 mg, 2.05 mmol) in acetonitrile (10 mL) was added-thionyl chloride (0.75 mL, mmol). The reaction mixture was allowed to stir at room temperature for 3 hours and then concentrated onto silica gel in vacuo. The residue was purified by flash chromatography (MPLC, 0-20% MeOH/DCM) to obtain 5-[3-(chloromethyl)-4-oxopyridazin-1(4H)-yl]pyridine-3-carbonitrile as a yellow solid.

LRMS (ESI) calc'd for C11H7ClN4O [M+H]⁺: 247. Found: 247.

The following intermediate was prepared from 1-(4-bromo-3-fluorophenyl)-3-(hydroxymethyl)pyridazin-4(1H)-one (prepared from 4-bromo-3-fluoroaniline according to Scheme 1) according to Scheme 2 following similar procedures described for Intermediate #25, which can be achieved by those of ordinary skill in the art of organic synthesis.

| | | | Exact |
|---|---|---|---|
| Inter- | | | Mass |
| medi- | | | [M + |
| ate | Structure | IUPAC Name | H]⁺ |
| 26 | 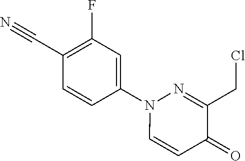 | 4-[3- (chloromethyl)- 4- oxopyridazin- 1(4H)-yl]-2- fluorobenzo- nitrile | Calc'd 264, found 264 |

### Scheme 3

### Intermediate #27

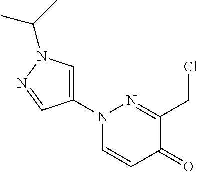

### 3-(Chloromethyl)-1-[1-(propan-2-yl)-1H-pyrazol-4-yl]pyridazin-4(1H)-one

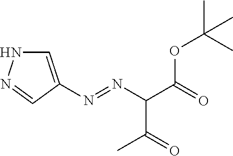

### Step 1. tert-Butyl 3-oxo-2-[1H-pyrazol-4-yldiazenyl]butanoate

1H-Pyrazol-4-amine (10.4 g, 125 mmol) was dissolved in concentrated HCl (36.8 mL)/water (186 mL) and cooled to 0° C. A solution of sodium nitrite (9.05 g, 131 mmol) in water (122 mL) was added dropwise while maintaining the internal temperature below 4° C. On complete addition, the mixture was stirred at 0° C. for 30 min. The resulting diazonium chloride solution was added via a pipette to a solution of tert-butyl acetoacetate (21.8 mL, 131 mmol) and sodium acetate (124 g, 1.51 mol) in water (122 mL) and EtOH (122 mL) at 0° C. The resulting mixture was stirred between 0-15° C. for two hours. The solid was filtered and dried in vacuo to afford tert-butyl 3-oxo-2-[1H-pyrazol-4-yldiazenyl]butanoate as a yellow-brownish powder.

LRMS (ESI)-calc'd for C11H17N4O3 [M+H]⁺: 253. Found: 253.

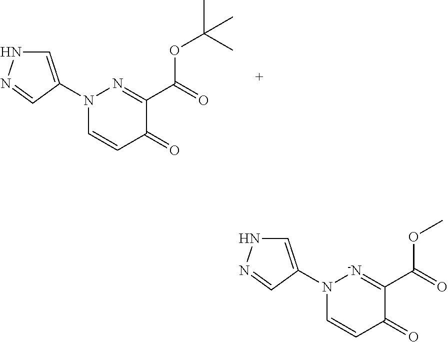

### Step 2. tert-Butyl 4-oxo-1-1H-pyrazol-4-yl)-1,4-dihydropyridazine-3-carboxylate and methyl-1-(1H-pyrazol-4-yl)-4-oxo-1,4-dihydropyridazine-3-carboxylate

tert-Butyl 3-oxo-2-[1H-pyrazol-4-yldiazenyl]butanoate (29.8 g, 118 mmol) was stirred in DMFDMA (236 mL) at 90° C. for 45 minutes. After cooling to room temperature, the solvent was removed in vacuo. The mixture was separated by flash chromatography (MPLC, 0-15% MeOH-DCM) to afford tert-butyl-1-(1H-pyrazol-4-yl)-4-oxo-1,4-dihydropyridazine-3-carboxylate and methyl-1-(1H-pyrazol-4-yl)-4-oxo-1,4-dihydropyridazine-3-carboxylate.

LRMS (ESI) calc'd for C12H15N4O3 [M+H]⁺: 263. Found: 263.

LRMS (ESI) calc'd for C9H9N4O3 [M+H]⁺: 221. Found: 221.

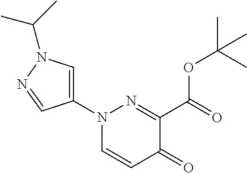

### Step 3. tert-Butyl 4-oxo-1-[1-(propan-2-yl)-1H-pyrazol-4-yl]-1,4-dihydropyridazine-3-carboxylate

To a solution of tert-butyl-1-(1H-pyrazol-4-yl)-4-oxo-1,4-dihydropyridazine-3-carboxylate (600 mg, 2.29 mmol) in DMF (7.6 mL) was added Cs₂CO₃ (894 mg, 2.75 mmol) and 2-iodopropane (0.275 mL, 2.75 mmol) in a microwave vial under nitrogen. The mixture was allowed to react in the microwave for 45 min at 110° C. Upon completion, the reaction mixture was diluted with EtOAc, washed with water and the aqueous phase was extracted with EtOAc (3×). The organic fractions were concentrated in vacuo and purified by flash chromatography (MPLC, 0-15% MeOH-DCM) to afford tert-butyl 4-oxo-1-[1-(propan-2-yl)-1H-pyrazol-4-yl]-1,4-dihydropyridazine-3-carboxylate.

LRMS (ESI) calc'd for C15H20N4O3 [M+H]⁺: 305. Found: 305.

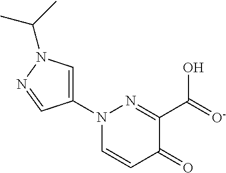

### Step 4. 4-Oxo-1-[1-(propan-2-yl)-1H-pyrazol-4-yl]-1,4-dihydropyridazine-3-carboxylic acid

To a solution of tert-butyl 4-oxo-1-[1-(propan-2-yl)-1H-pyrazol-4-yl]-1,4-dihydropyridazine-3-carboxylate (492 mg, 1.62 mmol) in DCM (7.3 mL) was added TFA (734 μL) and the mixture was allowed to stir at room temperature. After 2 hrs, the reaction mixture was concentrated in vacuo. To the residue was added diethyl ether and the slurry was stirred for minutes. The product was filtered and used in the next step without further purification.

LRMS (ESI) calc'd for C11H13N4O3 [M+H]⁺: 249. Found: 249.

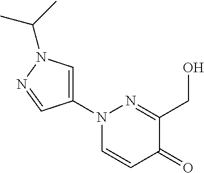

### Step 5. 3-(Hydroxymethyl)-1-[1-(propan-2-yl)-1H-pyrazol-4-yl]pyridazin-4(1H)-one

To 4-oxo-1-[1-(propan-2-yl)-1H-pyrazol-4-yl]-1,4-dihydropyridazine-3-carboxylic acid (324 mg, 1.31 mmol) in THF (6.5 mL), was added isobutyl chloroformate (206 L, 1.57 mmol) and N-methylmorpholine (172 μL, 1.57 mmol) and the mixture was allowed to stir for 20 min. Subsequently NaBH₄ (148 mg, 3.92 mmol) in water (651 μL) was added and stirred for 30 min. Upon complete conversion, the reaction was quenched with water and the solvent removed in vacuo while loading onto silica. Purification of the residue by flash chromatography (MPLC, 0-15% MeOH-DCM) afforded 3-(hydroxymethyl)-1-[1-(propan-2-yl)-1H-pyrazol-4-yl]pyridazin-4(1H)-one as a yellow oil.

LRMS (ESI) calc'd for C11H15N4O2 [M+H]⁺: 235. Found: 235.

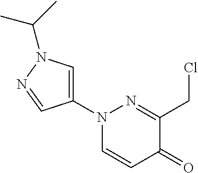

### Step 6. 3-(Chloromethyl)-1-[1-(propan-2-yl)-1H-pyrazol-4-yl]pyridazin-4(1H)-one

To 3-(hydroxymethyl)-1-[1-(propan-2-yl)-1H-pyrazol-4-yl]pyridazin-4(1H)-one (233 mg, 0.995 mmol) in MeCN (9.9 mL) was added thionyl chloride (363 μL, 4.97 mmol) and the mixture allowed to stir at r.t. After 1.5 hrs the reaction mixture was concentrated in vacuo while loading onto silica. Purification of the residue by flash chromatography (MPLC, 0-12% MeOH-DCM) to provide ethyl [3-({4-oxo-1-[1-(propan-2-yl)-1H-pyrazol-4-yl]-1,4-dihydropyridazin-3-yl}methyl)phenyl]carbamate.

LRMS (ESI) calc'd for C11H14ClN4O [M+H]⁺: 253. Found: 253.

The following intermediates were prepared according to Scheme 3 following similar procedures described for Intermediate #27, which can be achieved by those of ordinary skill in the art of organic synthesis.

| | | | Exact Mass |
|---|---|---|---|
| Intermediate | Structure | IUPAC Name | [M + H]⁺ |
| 28 | 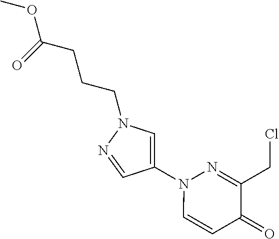 | methyl 4-{4-[3- (chloromethyl)-4- oxopyridazin-1(4H)-yl]- 1H-pyrazol-1- yl}butanoate | Calc'd 311, found 311 |
| 29 | 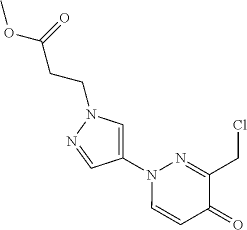 | methyl 3-{4-[3- (chloromethyl)-4- oxopyridazin-1(4H)-yl]- 1H-pyrazol-1- yl}propanoate | Calc'd 297, found 297 |
| 30 | 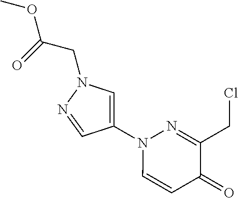 | methyl {4-[3- (chloromethyl)-4- oxopyridazin-1(4H)-yl]- 1H-pyrazol-1-yl}acetate | Calc'd 283, found 283 |
| 31 | 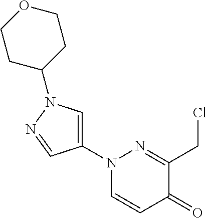 | 3-(chloromethyl)-1-[1- (tetrahydro-2H-pyran-4- yl)-1H-pyrazol-4- yl]pyridazin-4(1H)-one | Calc'd 295, found 295 |
| 32 | 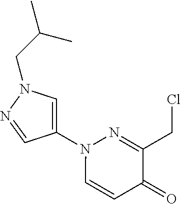 | 3-(chloromethyl)-1-(1- isobutyl-1H-pyrazol-4- yl]pyridazin-4(1H)-one | Calc'd 267, found 267 |
| 33 | 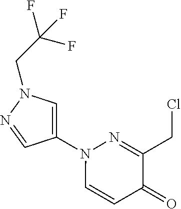 | 3-(chloromethyl)-1-[1- (2,2,2-trifluoroethyl)- 1H-pyrazol-4- yl]pyridazin-4(1H)-one | Calc'd 293, found 293 |
| 34 | 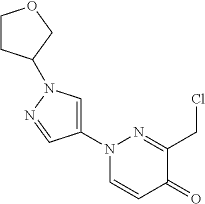 | rac-3-(chloromethyl)-1- [1-(tetrahydrofuran-3- yl)-1H-pyrazol-4- yl]pyridazin-4(1H)-one | Calc'd 281, found 281 |
| 35 | 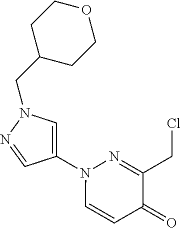 | 3-(chloromethyl)-1-[1- (tetrahydro-2H-pyran-4- ylmethyl)-1H-pyrazol-4- yl]pyridazin-4(1H)-one | Calc'd 309, found 309 |
| 36 | 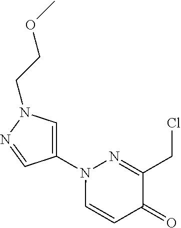 | 3-(chloromethyl)-1-[1- (2-methoxyethyl)-1H- pyrazol-4-yl]pyridazin- 4(1H)-one | Calc'd 269, Found 269 |
| 37 | 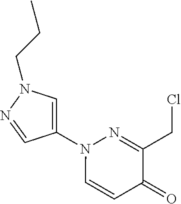 | 3-(chloromethyl)-1-(1- propyl-1H-pyrazol-4- yl]pyridazin-4(1H)-one | Calc'd 253, Found 253 |
| 38 | 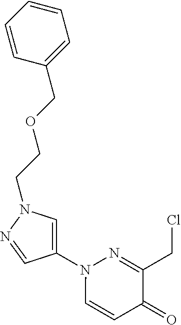 | 1-{1-[2- (benzyloxy)ethyl]-1H- pyrazol-4-yl}-3- (chloromethyl)pyridazin- 4(1H)-one | Calc'd 345, Found 345 |

### Intermediate #39

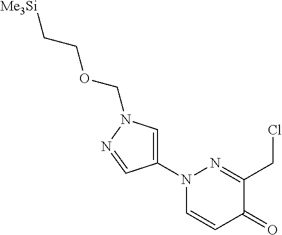

### 3-(Chloromethyl)-1-(1-{[2-(trimethylsilyl)ethoxy]methyl}-1H-pyrazol-4-yl)pyridazin-4(1H)-one

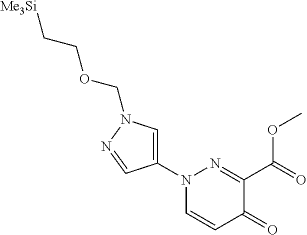

### Step 1. Methyl 4-oxo-1-(1-{[2-(trimethylsilyl)ethoxy]methyl}-1H-pyrazol-4-yl)-1,4-dihydropyridazine-3-carboxylate

To a solution of methyl 4-oxo-1-(1H-pyrazol-4-yl)-1,4-dihydropyridazine-3-carboxylate (Intermediate #27 Step 2, 4g, 18.1 mmol) in DMF (60.6 mL) was added NaH (60 wt %, 0.87 g, 21.8 mmol). The reaction was stirred for 30 minutes at room temperature and SEM-C1 (3.85 mL, 21.8 mmol) was added dropwise. The reaction mixture was stirred for 14 hrs. Upon complete conversion, the reaction was quenched with water, the aqueous phase was extracted with EtOAc (X4), the combined organic phases were dried over Na₂SO₄ and the solvent was removed in vacuo. The residue was purified by flash chromatography (MPLC, 0-10% MeOH-DCM) to provide methyl 4-oxo-1-(1-{[2-(trimethylsilyl)ethoxy]methyl}-1H-pyrazol-4-yl)-1,4-dihydropyridazine-3-carboxylate.

LRMS (ESI) calc'd for C15H23N4O4Si [M+H]⁺: 351. Found: 351.

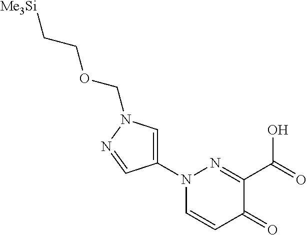

### Step 2. 4-Oxo-1-(1-{[2-(trimethylsilyl)ethoxy]methyl}-1H-pyrazol-4-yl)-1,4-dihydropyridazine-3-carboxylic acid

Methyl 4-exo-1-(1-{[2-(trimethylsilyl)ethoxy]methyl}-ii-pyrazol-4-yl)-1,4-dihydropyridazine-3-carboxylate (3.62 g, 10.3 mmol) and LiOH (1M in water, 13.9 mL, 13.9 mmol) were stirred in THF (5.7 mL) and MeOH (0.57 mL) at room temperature for 2 hrs. 1 N HCl was added and the pH was adjusted to 7. The aqueous phase was extracted with EtOAc (X4), the combined organic phases were washed with brine, dried over Na₂SO₄, filtered and concentrated in vacuo to give 4-oxo-1-(1H-pyrazol-4-yl)-1,4-dihydropyridazine-3-carboxylic acid.

LRMS (ESI) calc'd for C14H21N4O4Si [M+H]⁺: 337. Found: 337.

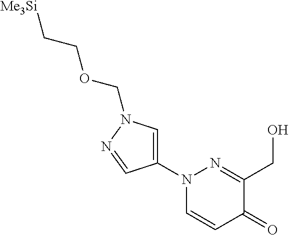

### Step 3. 3-(Hydroxymethyl)-1-(1-{2-(trimethylsilylethoxy)methyl}-1H-pyrazol-4-yl)pyridazin-4(1H)-one

To a solution of 4-oxo-1-(1H-pyrazol-4-yl)-1,4-dihydropyridazine-3-carboxylic acid (500 mg, 1.48 mmol) in THF (14.9 mL) was added isobutyl chloroformate (0.23 mL, 1.78 mmol) followed by N-methylmorpholine (0.19 mL, 1.78 mmol) at 0° C. and the reaction mixture was stirred for 30 min. To the reaction mixture was added NaBH₄ (169 mg, 4.46 mmol) in water (0.95 mL) and the reaction was stirred for an additional hour. Water was added, the reaction mixture was extracted with EtOAc (4×), the combined organic phases were dried over Na₂SO₄, the solvent was removed in vacuo and the residue was purified by flash chromatography (MPLC, 0-20% MeOH-DCM) to provide 3-(hydroxymethyl)-1-(1-{[2-(trimethylsilyl)ethoxy]methyl}-1H-pyrazol-4-yl)pyridazin-4(1H)-one.

LRMS (ESI) calc'd for C14H23N4O3Si [M+H]⁺: 323. Found: 323.

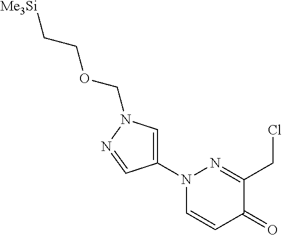

### Step 4. 3-(Chloromethyl)-1-(1-{[2-(trimethylsilyl)ethoxy]methyl}-1H-pyrazol-4-yl)pyridazin-4(1H)-one

To a solution of 3-(hydroxymethyl)-1-(1-{[2-(trimethylsilyl)ethoxy]methyl}-1H-pyrazol-4-yl)pyridazin-4(1H)-one (376 mg, 1.16 mmol) in MeCN (11.7 mL) was added K₂CO₃ (483 mg, 3.50 mmol) followed by the dropwise addition of SOCl₂ (0.25 mL, 3.50 mmol). Upon complete conversion, saturated aqueous NaHCO₃ was added, the aqueous phase was extracted with EtOAc (4×), the combined-organic phases were dried over Na₂SO₄, the solvent was removed in vacuo and the residue was purified by flash chromatography (MPLC, 0-15% MeOH-DCM) to provide 3-(chloromethyl)-1-(1-{[2-(trimethylsilyl)ethoxy]methyl}-1H-pyrazol-4-yl)pyridazin-4(1H)-one.

LRMS (ESI) calc'd for C14H22ClN4O2Si [M+H]⁺: 341. Found: 341.

### Boronic Ester Synthesis Method A

### Intermediate #40

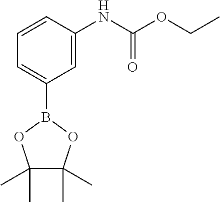

### Ethyl [3-(4,4,5,5-tetramethyl-1,3,2-dioxaborolan-2-yl)phenyl]carbamate

### Step 1. Ethyl [3-(4,4,5,5-tetramethyl-3,2-dioxaborolan-2-yl phenyl]carbamate

To a 50 mL round bottom flask was added 3-(4,4,5,5-tetramethyl-1,3,2-dioxaborolan-2-yl)aniline (8.00 g, 35.4 mmol) and DIPEA (7.4 mL, 43 mmol) in THF (30 mL). The mixture was cooled to 0° C. Ethyl chloroformate (7.42 mL, 42.5 mmol) was added slowly, and the reaction was stirred overnight while warming to room temperature. The reaction mixture was diluted with DCM and washed saturated NaHCO₃. The organic layer was concentrated in vacuo and dried on the lyophilizer to afford ethyl [3-(4,4,5,5-tetramethyl-1,3,2-dioxaborolan-2-yl)phenyl]carbamate a white solid.

The following intermediates were prepared according to Method A following similar procedures described for Intermediate #40, which can be achieved by those of ordinary skill in the art of organic synthesis.

| | | | Exact Mass |
|---|---|---|---|
| Intermediate | Structure | IUPAC Name | [M + H]+ |
| 41 | 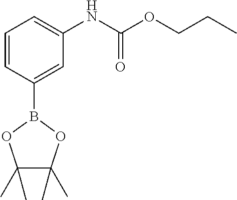 | Propyl [3-(4,4,5,5- tetramethyl-1,3,2- dioxaborolan-2- yl)phenyl]carbamate | Calc'd 306 found 306 |
| 42 | 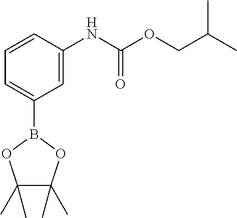 | isobutyl [3-(4,4,5,5- tetramethyl-1,3,2- dioxaborolan-2- yl)phenyl]carbamate | Calc'd 320 found 320 |
| 43 | 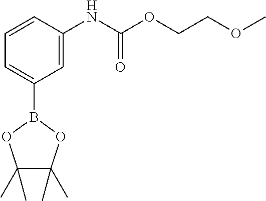 | 2-methoxyethyl [3- (4,4,5,5-tetramethyl- 1,3,2-dioxaborolan- 2- yl)phenyl]carbamate | Calc'd 322 found 322 |

### Boronic Ester Synthesis Method A

### Intermediate #44

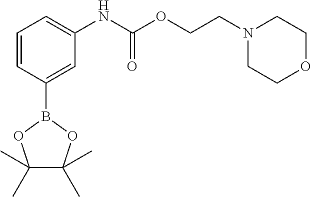

### 2-(Morpholin-4-yl)ethyl [3-(4,4,5,5-tetramethyl-1,3,2-dioxaborolan-2-yl)phenyl]carbamate

### Step 1. 2-(Morpholin-4-yl)ethyl [3-(4,4,5,5-tetramethyl-1,3,2-dioxaborolan-2-yl)phenyl]carbamate

N-(2-Hydroxyethyl)morpholine (29.9 g, 228 mmol) was dissolved in MeCN (45 mL). Separately, N,N-disuccinimidyl carbonate (58.5 g, 228 mmol) and TEA (63 mL, 460 mmol) were combined in MeCN (234 mL) and stirred for 20 min. The resulting slurry was then poured into the alcohol solution and MeCN was added until it became a dark solution. A slow exotherm to 29° C. was observed. After 4 hr, a solution of 3-(4,4,5,5-tetramethyl-1,3,2-dioxaborolan-2-yl)aniline in MeCN (190 mL) was added and the reaction was aged overnight. The reaction was then concentrated and purified on silica (0-15% MeOH/DCM). The semi-pure material (87.0 g) was dissolved in THF (400 mL) and cooled to 8° C. followed by the addition of K₂CO₃ (90 mL 10% aqueous, 1 equiv), EtOAc (850 mL) and water (300 mL). After the separation, the aqueous layer was extracted with EtOAc and the combined extracts were washed with water, dried over MgSO₄, filtered and concentrated to 31.0 g oil. As it solidified, it was slurried in DCM, and heptane (400 mL) was added. The volume was then concentrated to 250 mL and the slurry was filtered and washed with heptane to afford the product as a beige powder.

LRMS (ESI) calc'd for (C19H29BN2O5) [M+H]⁺: 377. Found: 377.

The following intermediate was prepared according to Method A following a similar procedure described for Intermediate #44, which can be achieved by those of ordinary skill in the art of organic synthesis.

| | | | Exact |
|---|---|---|---|
| | | | Mass |
| Intermediate | Structure | IUPAC Name | [M + H]+ |
| 45 | 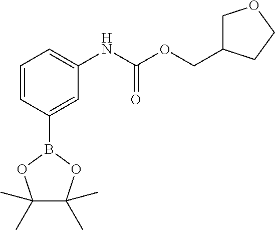 | rac-tetrahydrofuran- 3-ylmethyl [3- (4,4,5,5-tetramethyl- 1,3,2-dioxaborolan- 2- yl)phenyl]carbamate | Calc'd 348, Found 348 |

### Boronic Ester Synthesis Method B

### Intermediate #46

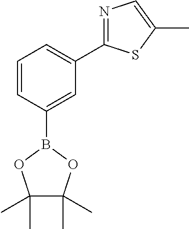

### 5-Methyl-2-[3-(4,4,5,5-tetramethyl-1,3,2-dioxaborolan-2-yl)phenyl]-1,3-thiazole

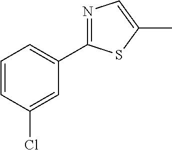

### Step 1. 2-(3-Chlorophenyl)-5-methyl-13-thiazole

To a 20 mL microwave vial was added (3-chlorophenyl)boronic acid (512 mg, 3.28 mmol), 2-bromo-5-methyl-1,3-thiazole (0.49 mL, 2.7 mmol), K₂CO₃ (754 mg, 5.46 mmol), dioxane (9 mL), PdCl₂(dppf).DCM (100 mg, 0.136 mmol), and water (1 mL). The vial was sealed and stirred at 50° C. overnight. The crude product was dissolved in EtOAc, filtered through Celite and concentrated in vacuo. The residue was absorbed onto silica and purified by flash chromatography (MPLC, 30-100% EtOAc-Hexanes) to provide 2-(3-chlorophenyl)-5-methyl-1,3-thiazole.

LRMS (ESI) calc'd for C10H9ClNS [M+H]⁺: 210. Found: 210.

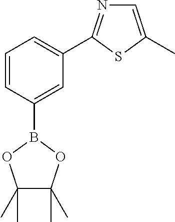

### Step 2. 5-Methyl-2-[3-(4,4,5,5-tetramethyl-1,3,2-dioxaborolan-2-yl)phenyl]-1,3-thiazole

To a 5 mL vial was added bis(pinacolato)diboron (452 mg, 1.78 mmol), KOAc (350 mg, 3.56 mmol), XPhos (57 mg, 0.12 mmol), and 2-(3-chlorophenyl)-5-methyl-1,3-thiazole-(249 mg, 1.19 mmol) in dioxane (6 mL). Pd₂(dba)₃ (27 mg, 0.030 mmol) was added last and the vial was purged with nitrogen for 5 minutes. The vial was sealed and heated to 100° C. overnight. The crude reaction mixture was filtered through Celite and the solvent removed in vacuo while loading onto silica. Purification of the residue by flash chromatography (MPLC, 0-50% EtOAc-Hexanes) gave 5-methyl-2-[3-(4,4,5,5-tetramethyl-1,3,2-dioxaborolan-2-yl)phenyl]-1,3-thiazole.

LRMS (ESI) calc'd for C16H21BNO2S [M+H]⁺: 302. Found: 302.

### Boronic Ester Synthesis Method RB

### Intermediate #47

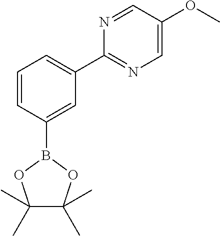

### 5-Methoxy-2-[3-(4,4,5,5-tetramethyl-1,3,2-dioxaborolan-2-yl)phenyl]pyrimidine

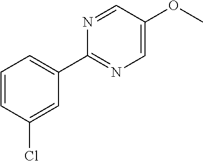

### Step 1. 2-(3-Chlorophenyl)-5-methoxypyrimidine

2-Chloro-5-methoxypyrimidine (0.96 g, 6.62 mmol), PdCl₂(dppf).DCM adduct (0.54 g, 0.66 mmol), Na₂CO₃ (6.62 mL, 13.24 mmol) and 3-chlorophenylboronic acid (1.55 g, 9.93 mmol) were taken up in 1,4-dioxane (9.6 mL). The flask was evacuated and back-filled with N₂ (X3) before stirring at 100° C. for 75 minutes. Room temperature was attained, saturated NH₄Cl was added and the products extracted into EtOAc (X2). The combined organic extracts were washed with brine, dried over MgSO₄, filtered through Celite and concentrated in vacuo. Purification of the residue by flash chromatography (MPLC, 1-20% EtOAc-hexanes) gave 2-(3-chlorophenyl)-5-methoxypyrimidine as a white solid.

LRMS (ESI) calc'd for C11H10ClN20 [M+H]⁺: 221. Found: 221.

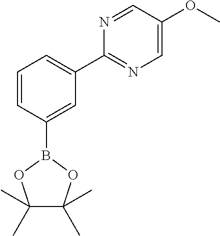

### Step 2. 5-Methoxy-2-[3-(4,4,5,5-tetramethyl-1,3,2-dioxaborolan-2-yl)phenyl]pyrimidine

2-(3-Chlorophenyl)-5-methoxypyrimidine (1.29 g, 5.85 mmol), Pd₂(dba)₃ (0.107 g, 0.117 mmol), X-Phos (0.223 g, 0.468 mmol), bis(pinacolato)diboron (1.78 g, 7.02 mmol) and KOAc (1.15 g, 11.7 mmol) were taken up in 1,4-dioxane (13 mL). The flask was evacuated and back-filled with N₂ (X3) before heating to 100° C. for 4 hours. Room temperature was attained, saturated NH₄Cl was added and the products extracted into EtOAc (X3). The combined organic extracts were washed with brine, dried over Na₂SO₄, filtered through Celite and concentrated in vacuo. The residue was triturated in hexanes to give 5-methoxy-2-[3-(4,4,5,5-tetramethyl-1,3,2-dioxaborolan-2-yl)phenyl]pyrimidine as an off-white solid.

LRMS calc'd for C17H22BN2O3 [M+H]⁺: 313. Found: 313

The following intermediates were prepared according to Method B following similar procedures described for Intermediates #46 and 47, which can be achieved by those of ordinary skill in the art of organic synthesis.

| | | | Exact |
|---|---|---|---|
| Inter- | | | Mass |
| medi- | | IUPAC | [M + |
| ate | Structure | Name | H]+ |
| 48 | 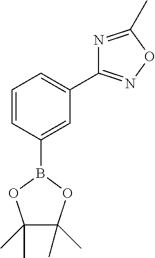 | 5-methyl-3- [3-(4,4,5,5- tetramethyl- 1,3,2- dioxaborolan- 2-yl)phenyl]- 1,2,4- oxadiazole | Calc'd 287, found 287 |
| 49 | 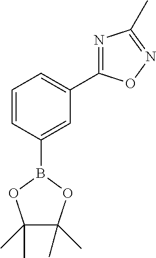 | 3-methyl-5- [3-(4,4,5,5- tetramethyl- 1,3,2- dioxaborolan- 2-yl)phenyl]- 1,2,4- oxadiazole | Calc'd 287, found 287 |
| 50 | 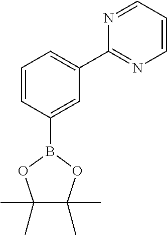 | 2-[3-(4,4,5,5- tetramethyl- 1,3,2- dioxaborolan- 2-yl)phenyl] pyrimidine | Calc'd 283, found 283 |
| 51 | 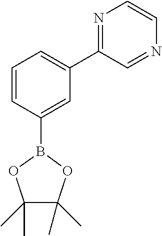 | 2-[3-(4,4,5,5- tetramethyl- 1,3,2- dioxaborolan- 2-yl)phenyl] pyrazine | Calc'd 283, found 283 |
| 52 | 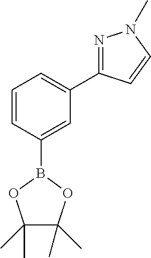 | 1-methyl-3- [3-(4,4,5,5- tetramethyl- 1,3,2- dioxaborolan- 2-yl)phenyl]- 1H-pyazole | Calc'd 285, found 285 |
| 53 | 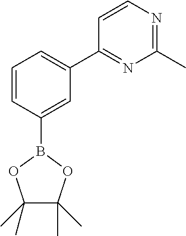 | 2-methyl-4- [3-(4,4,5,5- tetramethyl- 1,3,2- dioxaborolan- 2-yl)phenyl] pyrimidine | Calc'd 297, found 297 |
| 54 | 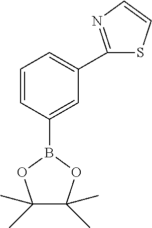 | 2-[3-(4,4,5,5- tetramethyl- 1,3,2- dioxaborolan- 2-yl)phenyl]- 1,3-thiazole | Calc'd 288, Found 288 |
| 55 | 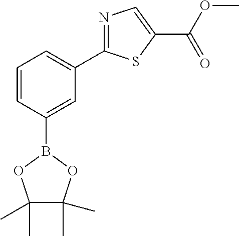 | Methyl-2-[3- (4,4,5,5- tetramethyl- 1,3,2- dioxaborolan- 2-yl)phenyl]- 1,3-thiazole- 5-carboxylate | Calc'd 346, Found 346 |

### Boronic Ester Synthesis Method B

### Intermediate #56

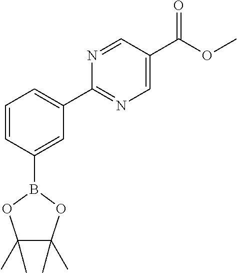

### Methyl 2-[3-(4,4,5,5-tetramethyl-1,3,2-dioxaborolan-2-yl)phenyl]pyrimidine-5-carboxylate

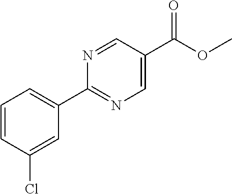

### Step 1. Methyl 2-(3-chlorophenyl)pyrimidine-5-carboxylate

To a 75 mL pressure vial was added sodium carbonate (1.23 g, 11.59 mmol), triphenylphosphine (300 mg, 1.159 mmol), palladium (II) acetate (52 mg, 0.232 mmol), methyl 2-chloropyrimidine-5-carboxylate (1.0 g, 5.79 mmol), (3-chlorophenyl)boronic acid (1.36 g, 8.69 mmol), and 1,4-dioxane (30 mL). The reaction vial was sealed and heated to 100° C. for 5 hours. Saturated NH₄Cl was added and the products extracted into EtOAc. The combined organics were concentrated in vacuo and the residue purified by flash chromatography (MPLC, 0-20% EtOAc-hexanes) to give methyl 2-(3-chlorophenyl)pyrimidine-5-carboxylate.

LRMS (ESI) calc'd for C12H10N2O2 [M+H]⁺: 249. Found: 249.

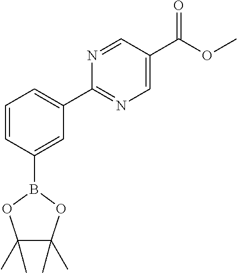

### Step 2. Methyl 2-[3-(4,4,5,5-tetramethyl-1,3,2-dioxaborolan-2-yl)phenyl]pyrimidine-5-carboxylate

Methyl 2-[3-(4,4,5,5-tetramethyl-1,3,2-dioxaborolan-2-yl)phenyl]pyrimidine-5-carboxylate was prepared from methyl. 2-(3-chlorophenyl)pyrimidine-5-carboxylate according to the procedure described for 5-ethoxy-2-[3-(4,4,5,5-tetramethyl-1,3,2-dioxaborolan-2-yl)phenyl]pyrimidine (Intermediate #86 Step 5).

LRMS calc'd for C18H22BN2O4 [M+H]⁺: 341. Found: 341

### Boronic Ester Synthesis Method B

### Intermediate #57

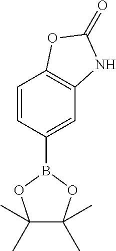

### 5-(4,4,5,5-Tetramethyl-1,3,2-dioxaborolan-2-yl)-1,3-benzoxazol-2(3H)-one

### Step 1. 5-(4,4,5,5-Tetramethyl-1,3,2-dioxaborolan-2-yl-1,3-benzoxazol-2(3H)-one

5-(4,4,5,5-Tetramethyl-1,3,2-dioxaborolan-2-yl)-1,3-benzoxazol-2(3H)-one was prepared from 5-bromo-1,3-benzoxazol-2(3H)-one according to the procedure described for 5-methyl-2-[3-(4,4,5,5-tetramethyl-1,3,2-dioxaborolan-2-yl)phenyl]-1,3-thiazole (Intermediate #46, Step 2).

LRMS (ESI) calc'd for C13H17BNO4 [M+H]⁺: 262. Found: 262.

The following intermediate was prepared according to Method B following similar procedures described for Intermediate #57, which can be achieved by those of ordinary skill in the art of organic synthesis.

| | | IUPAC | Exact Mass |
|---|---|---|---|
| Intermediate | Structure | Name | [M + H]+ |
| 58 | 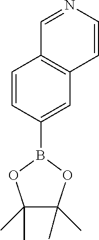 | 6-(4,4,5,5- tetramethyl- 1,3,2- dioxaborolan- 2- yl)isoquinoline | Calc'd 174, found 174 (boronic acid) |

### Boronic Ester Synthesis Method C

### Intermediate #59

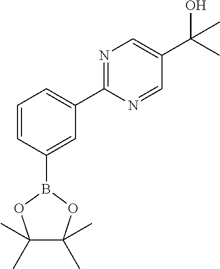

### 2-{2-[3-(4,4,5,5-Tetramethyl-1,3,2-dioxaborolan-2-yl)phenyl]pyrimidin-5-yl}propan-2-ol

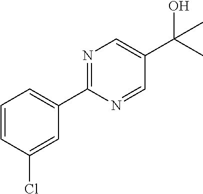

### Step 1. 2-[2-(3-Chlorophenyl)pyrimidin-5-yl]propan-2-ol

To an oven dried microwave vial was added methyl 2-(3-chlorophenyl)pyrimidine-5-carboxylate (Intermediate #56 Step 1, 0.665 g, 2.67 mmol) and THF (2.7 mL). The mixture was cooled to 0° C. and methyl magnesium bromide (3 M in THF, 2 mL, 5.88 mmol) was added. The reaction was allowed to warm to room temperature over the course of 3.5 hours, while stirring. Additional methylmagnesium bromide (3 M in THF, 668 μL, 2.005 mmol) was added and the reaction stirred for 20 hours. Water was added and the products extracted into ethyl acetate. The combined organics were concentrated in vacuo and the residue purified by flash chromatography (MPLC, 0-100% EtOAc-hexanes) to give 2-[2-(3-chlorophenyl)pyrimidin-5-yl]propan-2-ol.

LRMS (ESI) calc'd for C13H14ClN2O [M+H]⁺: 249. Found: 249.

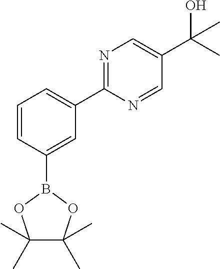

### Step 2. 2-{2-[3-(4,4,5,5-Tetramethyl-1,3,2-dioxaborolan-2-yl)phenyl]pyrimidin-5-yl}propan-2-ol

2-{2-[3-(4,4,5,5-Tetramethyl-1,3,2-dioxaborolan-2-yl)phenyl]pyrimidin-5-yl}propan-2-ol was prepared from 2-[2-(3-chlorophenyl)pyrimidin-5-yl]propan-2-ol according to the procedure described for 5-ethoxy-2-[3-(4,4,5,5-tetramethyl-1,3,2-dioxaborolan-2-yl)phenyl]pyrimidine (Intermediate #86 Step 5).

LRMS (ESI) calc'd for C19H26BN2O3 [M+H]⁺: 341. Found: 341.

### Boronic Ester Synthesis Method D

### Intermediate #60

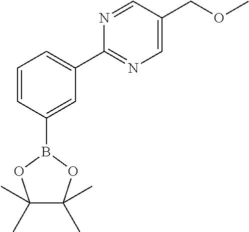

### 5-(Methoxymethyl)-2-[3-(4,4,5,5-tetramethyl-1,3,2-dioxaborolan-2-yl)phenyl]pyrimidine

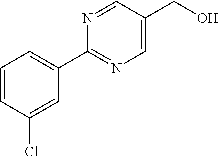

### Step 1. [2-(3-Chlorophenyl)pyrimidin-5-yl]methanol

To a 20 mL microwave vial equipped with a stir bar was added methyl 2-(3-chlorophenyl)pyrimidine-5-carboxylate (Intermediate #56 Step 1, 668 mg, 2.69 mmol) and THF (9 mL). The reaction mixture was cooled to 0° C. and DIBAL-H (3.5 mL, 3.51 mmol) was added dropwise. The reaction mixture was stirred at 0° C. for 2 hours. Additional DIBAL-H (2.7 mL) was added and the reaction was stirred for 3 hours. 2 N HCl was added followed by NaHCO₃ and the products extracted into ethyl acetate. The combined organics were concentrated in vacuo to give [2-(3-chlorophenyl)pyrimidin-5-yl]methanol.

LRMS (ESI) calc'd for C11H10ClN2O [M+H]⁺: 221. Found: 221.

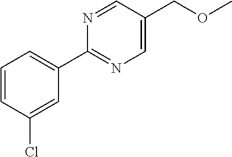

### Step 2. 2-(3-Chlorophenyl)-5-(methoxymethyl)pyrimidine

To a microwave vial was added [2-(3-chlorophenyl)pyrimidin-5-yl]methanol (193 mg, 0.875 mmol), DMF (96 mL) and iodomethane (55 μL, 0.875 mmol). The reaction mixture was stirred for 5 minutes, followed by the addition of sodium hydride (60 wt %, 25 mg, 1.05 mmol). The reaction was stirred for one hour. Additional iodomethane (55 μL, 0.875 mmol) was added and the reaction was stirred for one-hour. Water was added and the products extracted into EtOAc. The combined organics were concentrated in vacuo and the residue purified by flash chromatography (MPLC, 0-20% EtOAc-hexanes) to give 2-(3-chlorophenyl)-5-(methoxymethyl)pyrimidine.

LRMS (ESI) calc'd for C12H12ClN2O [M+H]⁺: 235. Found: 235.

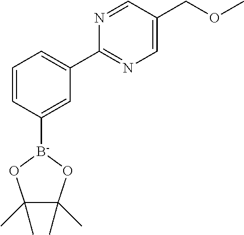

### Step 3. 5-(Methoxymethyl)-2-[3-(4,4,5,5-tetramethyl-1,3,2-dioxaborolan-2-yl)phenyl]pyrimidine

5-(Methoxymethyl)-2-[3-(4,4,5,5-tetramethyl-1,3,2-dioxaborolan-2-yl)phenyl]pyrimidine was prepared from 2-(3-chlorophenyl)-5-(methoxymethyl)pyrimidine according to the procedure described for 5-ethoxy-2-[3-(4,4,5,5-tetramethyl-1,3,2-dioxaborolan-2-yl)phenyl]pyrimidine (Intermediate #86 Step 5).

LRMS (ESI) calc'd for C18H24BN2O3 [M+H]⁺: 327. Found: 327.

The following intermediate was prepared according to Method D following similar procedures described for Intermediate #60, which can be achieved by those of ordinary skill in the art of organic synthesis.

| | | | Exact |
|---|---|---|---|
| | | | Mass |
| Intermediate | Structure | IUPAC Name | [M + H]+ |
| 61 | 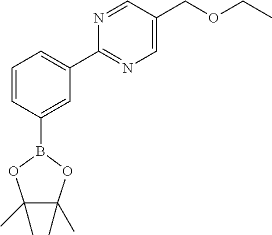 | 5-(ethoxymethyl)-2- [3-(4,4,5,5- tetramethyl-1,3,2- dioxaborolan-2- yl)phenyl]pyrimidine | Calc'd 341, found 341 |

### Boronic Ester Synthesis Method D

### Intermediate #62

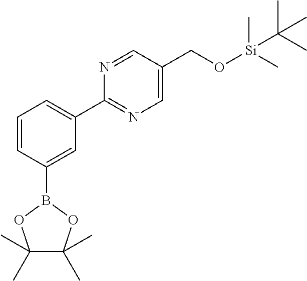

### 5-({[tert-Butyl(dim ethyl)silyl]oxy}methyl)-2-[3-(4,4,5,5-tetramethyl-1,3,2-dioxaborolan-2-yl)phenyl]pyrimidine

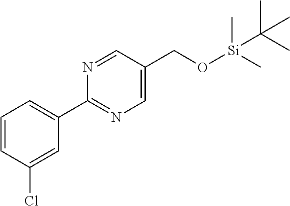

### Step 1. 5-({[tert-Butyl(dimethyl)silyl]oxy}methyl)-2-(3-chlorophenyl)pyrimidine

To a 5 mL microwave vial equipped with a stir bar was added [2-(3-chlorophenyl)pyrimidin-5-yl]methanol (340 mg, 1.5 mmol), imidazole (315 mg, 4.6 mmol), DMF (5 mL) and TBS-C1 (0.8 mL, 4.6 mmol). The vial was sealed and heated to 80° C. for 20 hours. The crude reaction mixture was concentrated in vacuo and carried on to the next step.

LRMS (ESI) calc'd for C17H24ClN2OSi [M+H]⁺: 335. Found: 335.

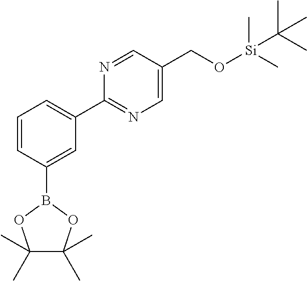

### Step 2. 5-({[tert-Butyl(dimethyl silyl]oxy}methyl-2-[3-(4,4,5,5-tetramethyl-1,3,2-dioxaborolan-2-yl)phenyl]pyrimidine

5-({[tert-Butyl(dimethyl)silyl]oxy}methyl)-2-[3-(4,4,5,5-tetramethyl-1,3,2-dioxaborolan-2-yl)phenyl]pyrimidine was prepared from 5-({[tert-butyl(dimethyl)silyl]oxy}methyl)-2-(3-chlorophenyl)pyrimidine according to the procedure described for 5-ethoxy-2-[3-(4,4,5,5-tetramethyl-1,3,2-dioxaborolan-2-yl)phenyl]pyrimidine (Intermediate #86 Step 5).

LRMS (ESI) calc'd for C23H36BN2O3Si [M+H]⁺: 427. Found: 427.

### Boronic Ester Synthesis Method E

### Intermediate #63

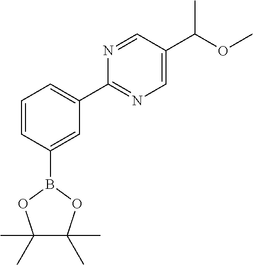

### rac-5-(1-Methoxyethyl)-2-[3-(4,4,5,5-tetramethyl-1,3,2-dioxaborolan-2-yl)phenyl]pyrimidine

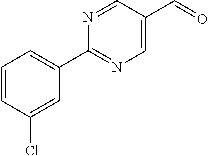

### Step 1. 2-(3-Chlorophenyl)pyrimidine-5-carbaldehyde

To a microwave vial equipped with a stir bar was added [2-(3-chlorophenyl)pyrimidin-5-yl]methanol (Intermediate #60 Step 1, 204 mg, 0.924 mmol) and DCM (4.6 mL). The reaction mixture was cooled to 0° C. Dess-Martin periodinane (431 mg, 1.02 mmol) was added and the reaction mixture was warmed to room temperature while stirring for 2.5 hours. Saturated sodium bicarbonate was added and the products extracted into EtOAc. The combined organics were concentrated in vacuo while loading onto silica. The residue was purified by flash chromatography (MPLC, 0-15% MeOH-DCM) to give 2-(3-chlorophenyl)pyrimidine-5-carbaldehyde.

LRMS (ESI) calc'd for C11H8ClN2O [M+H]⁺: 219. Found: 219.

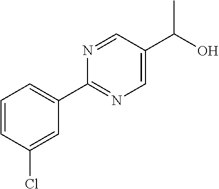

### Step 2. rac-1-[2-(3-Chlorophenyl)pyrimidin-5-yl]ethanol

To a microwave vial equipped with a stir bar was added 2-(3-chlorophenyl)pyrimidine-5-carbaldehyde (53 mg, 0.241 mmol) and THF (2.4 mL). The reaction mixture was cooled to −78° C. Methylmagnesium bromide (3 M in THF, 80 μL, 0.241 mmol) was added dropwise and the reaction was stirred for 15 minutes. The crude reaction mixture was concentrated in vacuo to give rac-1-[2-(3-chlorophenyl)pyrimidin-5-yl]ethanol that was used in the next step without purification.

LRMS (ESI) calc'd for C12H12ClN2O [M+H]⁺: 235. Found: 235.

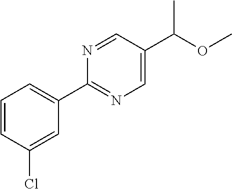

### Step 3. rac-2(3-Chlorophenyl)-5-(1-methoxyethyl)pyrimidine

To a microwave vial equipped with a stir bar was added rac-1-[2-(3-chlorophenyl)pyrimidin-5-yl]ethanol (60 mg, 0.26 mmol), iodomethane (18 L, 0.23 mmol) and DMF (2.6 mL) at room temperature. Sodium hydride (60 wt %, 11 mg; 0.28 mmol) was added last and the reaction was stirred for 1 hour at r.t. Additional iodomethane (16 μL, 0.26 mmol) and sodium hydride (60 wt %, 10 mg, 0.25 mmol) were added and the reaction stirred for two hours. Additional iodomethane (24 μL, 0.38 mmol) and sodium hydride (60 wt %, 15 mg, 0.38 mmol) were added and the reaction was stirred for 35 minutes. Additional iodomethane (32 μL, 0.51 mmol) and sodium hydride (60 wt %, 20 mg, 0.51 mmol) were added and the reaction was stirred for 2 hours. Saturated NaHCO₃ was added and the products extracted into EtOAc. The combined organics were concentrated in vacuo to give rac-2-(3-chlorophenyl)-5-(1-methoxyethyl)pyrimidine that was used in the next step without purification.

LRMS (ESI) calc'd for C13H14ClN2O [M+H]⁺: 249. Found: 249.

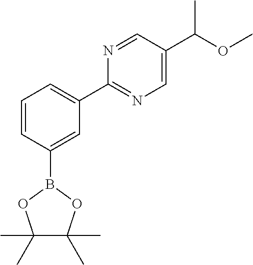

### Step 4. rac-5-(1-Methoxyethyl)-2-[3-(4,4,5-tetramethyl-1,3,2-dioxaborolan-2-yl)phenyl]pyrimidine

rac-5-(1-Methoxyethyl)-2-[3-(4,4,5,5-tetramethyl-1,3,2-dioxaborolan-2-yl)phenyl]pyrimidine was prepared from rac-2-(3-chlorophenyl)-5-(1-methoxyethyl)pyrimidine according to the procedure described for 5-ethoxy-2-[3-(4,4,5,5-tetramethyl-1,3,2-dioxaborolan-2-yl)phenyl]pyrimidine (Intermediate #86 Step 5).

LRMS (ESI) calc'd for C19H26BN2O3 [M+H]⁺: 341. Found: 341.

### Boronic Ester Synthesis Method F

### Intermediate #64

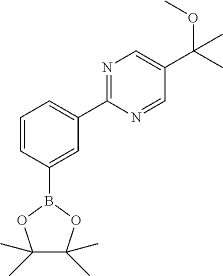

### 5-(1-Methoxy-1-methylethyl)-2-[3-(4,4,5,5-tetramethyl-1,3,2-dioxaborolan-2-yl)phenyl]pyrimidine

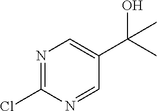

### Step 1 2-(2-Chloropyrimidin-5-yl)propan-2-ol

To an oven dried microwave vial equipped with a stir bar was added methyl 2-chloropyrimidine-5-carboxylate (516 mg, 2.99 mmol) and THF (30 mL). The reaction was cooled to 0° C. Methylmagnesium bromide (3 M in THF, 2.2 mL, 6.58 mmol) was added dropwise and the reaction was stirred for 3 hours. Saturated NaHCO₃ was added and the products extracted into ethyl acetate. The combined organics were concentrated in vacuo to give 2-(2-chloropyrimidin-5-yl)propan-2-ol.

LRMS (ESI) calc'd for C7H10ClN2O [M+H]⁺: 173. Found: 173.

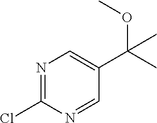

### Step 2. 2-Chloro-5-(1-methoxy-1-methylethyl)pyrimidine

2-(2-Chloropyrimidin-5-yl)propan-2-ol (132 mg, 0.765 mmol) and iodomethane (240 μL, 3.83 mmol) were taken up in DMF (5.1 mL). Sodium hydride (60 wt %, 37 mg, 0.918 mmol) was added and the reaction was stirred for 30 minutes. Water was added and the products extracted into ethyl acetate. The combined organics were concentrated in vacuo to give 2-chloro-5-(1-methoxy-1-methylethyl)pyrimidine.

LRMS (ESI) calc'd for C8H2ClN2O [M+H]⁺: 187. Found: 187.

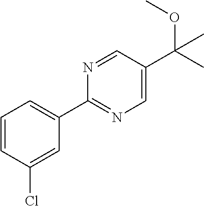

### Step 3. 2-(3-Chlorophenyl-5-(1-methoxy-1-methylethyl)pyrimidine

2-(3-Chlorophenyl)-5-(1-methoxy-1-methylethyl)pyrimidine was prepared from 2-chloro-5-(1-methoxy-1-methylethyl)pyrimidine according to the procedure described for 2-(3-chlorophenyl)-5-ethoxypyrimidine (Intermediate #86, Step 4).

LRMS (ESI) calc'd for C14H16ClN2O [M+H]⁺:263. Found: 263.

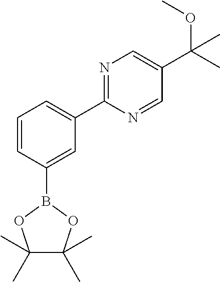

### Step 4. 5-(1-Methoxy-1-methylethyl)-2-[3-(4,4,5,5-tetramethyl-1,3,2-dioxaborolan-2-yl)phenyl]pyrimidine

5-(1-Methoxy-1-methylethyl)-2-[3-(4,4,5,5-tetramethyl-1,3,2-dioxaborolan-2-yl)phenyl]pyrimidine was prepared from 2-(3-chlorophenyl)-5-(1-methoxy-1-methylethyl)pyrimidine according to the procedure described for 5-ethoxy-2-[3-(4,4,5,5-tetramethyl-1,3,2-dioxaborolan-2-yl)phenyl]pyrimidine (Intermediate #86 Step 5).

LRMS (ESI) calc'd for C20H28BN2O3 [M+H]⁺: 355. Found: 355.

The following intermediate was prepared according to Method F following similar procedures described for Intermediate #64, which can be achieved by those of ordinary skill in the art of organic synthesis.

| Inter- | | | |
|---|---|---|---|
| medi- | | | Exact Mass |
| ate | Structure | IUPAC Name | [M + H]+ |
| 65 | 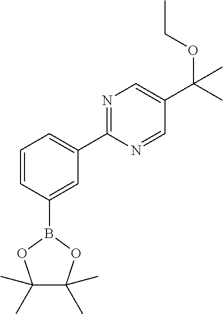 | 5-(1-ethoxy- 1- methylethyl)- 2-[3-(4,4,5,5- tetramethyl- 1,3,2- dioxaborolan- 2-yl)phenyl] pyrimidine | Calc'd 369, Found 369 |

### Boronic Ester Synthesis Method G

### Intermediate #66

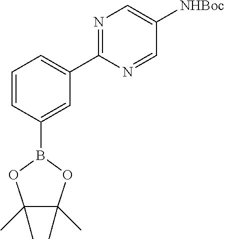

### tert-Butyl {2-[3-(4,4,5,5-tetramethyl-1,3,2-dioxaborolan-2-yl)phenyl]pyrimidin-5-yl}carbamate

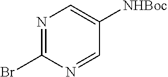

### Step 1. tert-Buty (2-bromopyrimidin-5-yl)carbamate

To a solution of 2-bromopyrimidin-5-amine (3.0 g, 23 mmol) in tert-BuOH (46 mL) was added Boc₂O (8.0 mL, 34 mmol). The reaction was stirred at 60° C. for two days, after which additional Boc₂O (8.0 mL, 34 mmol) was added and the reaction was kept at 60° C. for two days. Upon completion, the solvent was evaporated in vacuo and the residue was purified by flash chromatography (MPLC, 10-100% EtOAc-hexanes) to give tert-butyl (2-bromopyrimidin-5-yl)carbamate.

LRMS (ESI) calc'd for C9H13BrN3O2 [M+H]⁺: 274. Found: 274.

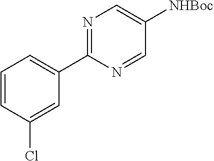

### Step 2. tert-Butyl [2-(3-chlorophenyl)pyrimidin-5-yl]carbamate

tert-Butyl [2-(3-chlorophenyl)pyrimidin-5-yl]carbamate was prepared from tert-butyl (2-bromopyrimidin-5-yl)carbamate according to the procedure described for 2-(3-chlorophenyl)-5-ethoxypyrimidine (Intermediate #86 Step 4).

LRMS (ESI) calc'd for C15H17ClN3O2 [M+H]⁺: 306. Found: 306.

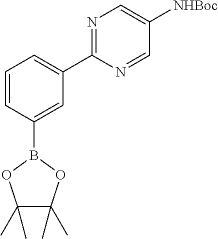

### Step 3. tert-Butyl {2-[3-(4,4,5,5-tetramethyl-1,3,2-dioxaborolan-2-yl)phenyl]pyrimidin-5-yl}carbamate

tert-Butyl {2-[3-(4,4,5,5-tetramethyl-1,3,2-dioxaborolan-2-yl)phenyl]pyrimidin-5-yl}carbamate was prepared as described for 5-ethoxy-2-[3-(4,4,5,5-tetramethyl-1,3,2-dioxaborolan-2-yl)phenyl]pyrimidine (Intermediate #86 Step 5).

LRMS (ESI) calc'd for C21H29N3BO4 [M+H]⁺: 398. Found: 398.

### Boronic Ester Synthesis Method H

### Intermediate #67

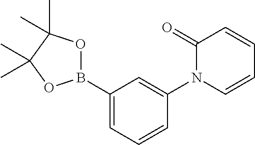

### 1-[3-(4,4,5,5-Tetramethyl-1,3,2-dioxaborolan-2-yl)phenyl]pyridin-2(1H)-one

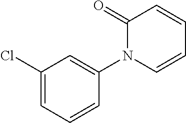

### Step 1. 1-(3-Chlorophenyl)pyridin-2(1H)-one

1-Chloro-3-iodobenzene (100 mg, 0.419 mmol), pyridin-2-ol (48 mg, 0.50 mmol), ethyl 2-cyclohexanonecarboxylate (14 mg, 0.084 mmol), copper(I) iodide (16 mg, 0.084 mmol), and Cs₂CO₃ (287 mg, 0.881 mmol) were combined in a 5 mL microwave vial. The vial was evacuated (X3) with N₂ gas before adding DMSO (4.5 mL). The mixture was allowed to stir at r.t. for 2 days. The mixture was filtered through Celite, eluting with MeOH/DCM, and loaded directly onto silica gel. Purification by flash chromatography (MPLC, 0-25% EtOAc-Hexanes) afforded 1-(3-chlorophenyl)pyridin-2(1H)-one.

LRMS (ESI) calc'd for C11H9ClNO [M+H]⁺: 206. Found: 206.

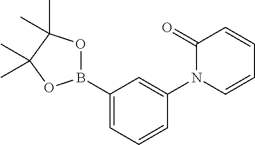

### Step 2. 1-[3-(4,5,5,5-Tetramethyl-1,3,2-dioxaborolan-2-yl)phenyl]pyridin-2(1H)-one

1-(3-Chlorophenyl)pyridin-2(1H)-one (115 mg, 0.559 mmol), B₂Pin₂ (213 mg, 0.839 mmol), Pd₂(dba)₃ (10 mg, 0.011 mmol), XPhos (0.045 mmol), and KOAc (165 mg; 1.68 mmol) were combined in a 5 mL microwave vial. The vial was evacuated and back-filled with N₂ gas (X3) before adding 1,4-dioxane (4.5 mL). The mixture was allowed to stir at 100° C. for 3 hours. The mixture was filtered through Celite, concentrated, and purified by flash chromatography (MPLC, 0-20% EtOAc-hexanes) to afford 1-[3-(4,4,5,5-tetramethyl-1,3,2-dioxaborolan-2-yl)phenyl]pyridin-2(1H)-one.

LRMS (ESI) calc'd for C17H20BNO3 [M+H]⁺: 298. Found: 298.

The following intermediate was prepared according to Method H following similar procedures described for Intermediate #67, which can be achieved by those of ordinary skill in the art of organic synthesis.

| Inter- | | | |
|---|---|---|---|
| medi- | | | Exact Mass |
| ate | Structure | IUPAC Name | [M + H]+ |
| 68 | 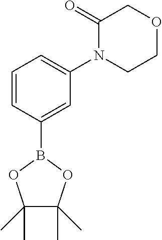 | 4-[3-(4,4,5,5- tetramethyl-1,3,2- dioxaborolan-2- yl)phenyl]morpholin- 3-one | Calc'd 304, found 304 |

### Boronic Ester Synthesis Method I

### Intermediate #69

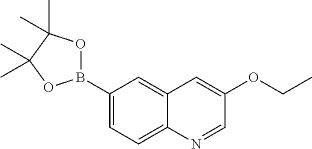

### 3-Ethoxy-6-(4,4,5,5-tetramethyl-1,3,2-dioxaborolan-2-yl)quinoline

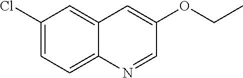

### Step 1. 6-Chloro-3-ethoxyquinoline

6-Chloroquinolin-3-ol (0.15 g, 0.84 mmol) was taken up in THF (4 mL) and NaH (0.037 g, 0.92 mmol) was added. After stirring for 15 minutes, iodoethane (0.074 mL, 0.92 mmol) was added and the resulting mixture stirred at r.t. for 1 hour. DMF (1 mL) was added to aid solubility and stirring at r.t. continued overnight. The solvent was removed in vacuo while loading onto silica and the residue purified by flash chromatography (MPLC, 12-100% EtOAc-Hexanes) to give 6-chloro-3-ethoxyquinoline as a white solid.

LRMS (ESI) calc'd for C11H11ClNO [M+H]⁺: 208, 210. Found: 208, 210.

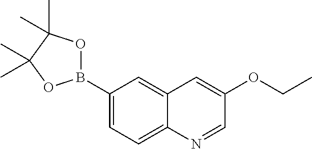

### Step 2. 3-Ethoxy-6-(4,4,5,5-tetramethyl-1,3,2-dioxaborolan-2-yl)quinoline

6-Chloro-3-ethoxyquinoline (122 mg, 0.588 mmol), bis(pinacolato)diboron (224 mg, 0.881 mmol), Pd₂(dba)₃ (10 mg, 0.012 mmol), XPhos (22 mg, 0.047 mmol) and KOAc (173 mg, 1.76 mmol) were stirred in 1,4-dioxane (3 mL) at 100° C. for 3 hours. After cooling to r.t. the solvent was removed in vacuo while loading onto silica. Purification of the residue by flash chromatography (MPLC, 6-50% EtOAc-Hexanes) gave 3-ethoxy-6-(4,4,5,5-tetramethyl-1,3,2-dioxaborolan-2-yl)quinoline as a pale yellow solid.

LRMS (ESI) calc'd for C17H23BNO3 [M+H]⁺: 300. Found: 300.

The following intermediates were prepared according to Method I following similar procedures described for Intermediate #69, which can be achieved by those of ordinary skill in the art of organic synthesis.

| | | | Exact Mass |
|---|---|---|---|
| Intermediate | Structure | IUPAC Name | [M + H]+ |
| 70 | 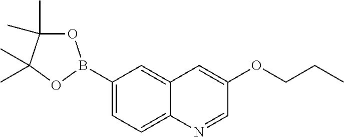 | 3-propoxy-6- (4,4,5,5- tetramethyl- 1,3,2- dioxaborolan-2- yl)quinoline | Calc'd 314, found 314 |
| 71 | 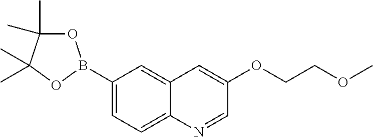 | 3-(2- methoxyethoxy)- 6-(4,4,5,5- tetramethyl- 1,3,2- dioxaborolan-2- yl)quinoline | Calc'd 248 (boronic acid), found 248 (boronic acid), |
| 72 | 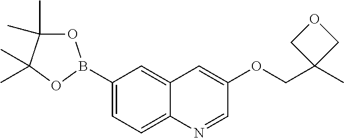 | 3-[(3- methyloxetan-3- yl)methoxy]-6- (4,4,5,5- tetramethyl- 1,3,2- dioxaborolan-2- yl)quinoline | Calc'd 274 (boronic acid), found 274 (boronic acid), |
| 73 | 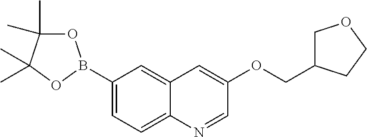 | rac-3- (tetrahydrofuran- 3-ylmethoxy)-6- (4,4,5,5- tetramethyl- 1,3,2- dioxaborolan-2- yl)quinoline | Calc'd 356, found 356 |

### Boronic Ester Synthesis Method I

### Intermediate #74

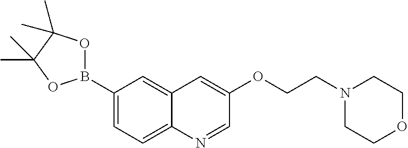

### 3-[2-(Morpholin-4-yl)ethoxy]-6-(4,4,5,5-tetramethyl-1,3,2-dioxaborolan-2-yl)quinoline

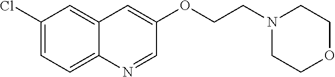

### Step 1. 6-Chloro-3-(2-morpholin-4-ylethoxy)quinoline

6-Chloroquinolin-3-ol (0.15 g, 0.84 mmol), N-(2-hydroxyethyl)morpholine-(0.153 mL, 1.25 mmol), triphenylphosphine (0.329 g, 1.25 mmol) and DIAD (0.24 mL, 1.3 mmol) were stirred in THF (4 mL) at r.t. for 3 hours. The solvent was removed in vacuo while loading onto silica. Purification of the residue by flash chromatography (MPLC, 0-10% MeOH-DCM) followed by flash chromatography (MPLC, 0-10% MeOH-EtOAc) gave 6-chloro-3-(2-morpholin-4-ylethoxy)quinoline as a white solid.

LRMS (ESI) calc'd for C15H18ClN2O2 [M+H]⁺: 293. Found: 293.

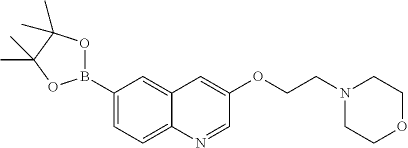

### Step 2. 3-[2-(Morpholin-4-yl)ethoxy]-6-(4,4,5,5-tetramethyl-1,3,2-dioxaborolan-2-yl)quinoline

3-[2-(Morpholin-4-yl)ethoxy]-6-(4,4,5,5-tetramethyl-1,3,2-dioxaborolan-2-yl)quinoline was prepared from 6-chloro-3-(2-morpholin-4-ylethoxy)quinoline according to the procedure described for 3-ethoxy-6-(4,4,5,5,5-tetramethyl-1,3,2-dioxaborolan-2-yl)quinoline (Intermediate #69 Step 2).

LRMS (ESI) calc'd for (C21H30BN2O4) [M+H]⁺: 385. Found: 385.

### Boronic Ester Synthesis Method J

### Intermediate #75

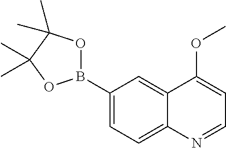

### 4-Methoxy-6-(4,4,5,5-tetramethyl-1,3,2-dioxaborolan-2-yl)quinoline

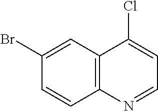

### Step 1. 4-Chloro-5-bromoquinoline

A 20 mL scintillation vial was charged with 6-bromoquinolin-4-ol (0.98 g, 4.4 mmol) and phosphorus (V) oxychloride (10 mL). The reaction mixture was stirred for 19 hours at ambient temperature and carefully poured into a mixture of saturated aqueous sodium bicarbonate solution and ice, extracted into ethyl acetate (3×), washed with brine, dried over magnesium sulfate, filtered and concentrated in vacuo. The residue was purified by flash chromatography (MPLC, 0-50% EtOAc-hexanes) to give 4-chloro-5-bromoquinoline.

LRMS (ESI) calc'd for C9H6BrClN [M+H]⁺: 242. Found: 242.

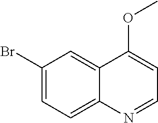

### Step 2. 6-Bromo-4-methoxyquinoline

A 5 mL microwave vial was charged with 4-chloro-6-bromoquinoline (0.15 g, 0.62 mmol) and a 25 wt % solution of sodium methoxide in methanol (2.0 mL, 8.8 mmol). The vial was sealed and heated to 100° C. for 60 minutes under microwave irradiation (Biotage, Initiator). After cooling, the solvent was removed in vacuo, the residue washed with water, filtered and dried via lyophilization to obtain 6-bromo-4-methoxyquinoline.

LRMS (ESI) calc'd for C10H9BrNO [M+H]⁺: 238. Found: 238.

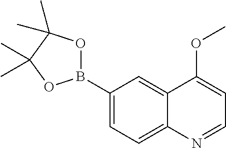

### Step 3. 4-Methoxy-6-(4,4,5,5-tetramethyl-1,3,2-dioxaborolan-2-yl)quinoline

4-methoxy-6-(4,4,5,5-tetramethyl-1,3,2-dioxaborolan-2-yl)quinoline was prepared from 6-bromo-4-methoxyquinoline according to the procedure described for 5-methyl-2-[3-(4,4,5,5-tetramethyl-1,3,2-dioxaborolan-2-yl)phenyl]-1,3-thiazole (Intermediate #39 Step 2).

LRMS (ESI) calc'd for C16H21BNO3 [M+H]⁺: 286. Found: 286.

### Boronic Ester Synthesis Method K

### Intermediate #76

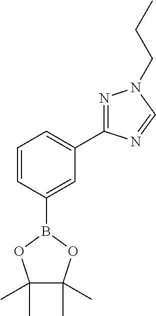

### 1-Propyl-3-[-(4,4,5,5-tetramethyl-1,3,2-dioxaborolan-2-yl)phenyl]-1-1H-1,2,4-triazole

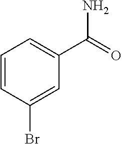

### Step 1. 3-Bromobenzamide

Into a 5000-mL 4-necked round-bottom flask purged and maintained with an inert atmosphere of nitrogen was placed a solution of 3-bromobenzoic acid (100 g, 497 mmol) in tetrahydrofuran (2000 mL), followed by the addition of N,N-carbonyldiimidazole (120.9 g, 746 mmol) in several batches at 0° C. The resulting solution was stirred overnight at room temperature, then NH₃ (g) was bubbled slowly into the reaction mixture at <10° C. for about 6 hours. The resulting mixture was concentrated under vacuum. The residue was dissolved in DCM (2 L) then washed with 5% HCl (2×1000 mL) and aqueous sodium carbonate (3×1000 mL). The organic phase was dried over anhydrous sodium sulfate and concentrated under vacuum to give 3-bromobenzamide as a white solid.

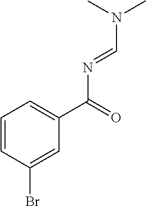

### Step 2. 3-Bromo-N-[(1E)-(dimethylamino)methylene]benzamide

Into a 2000-mL 3-necked round-bottom flask was placed a solution of 3-bromobenzamide (78 g, 390 mmol) in DMFDMA (800 mL). The resulting solution was stirred for 30 min at 90° C. in an oil bath. The resulting mixture was cooled and concentrated in vacuo to give 3-bromo-N-[(1E)-(dimethylamino)methylene]benzamide as a white solid.

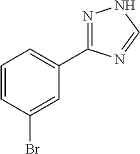

### Step 3. 3-(3-Bromophenyl)-1H-1,2,4-triazole

Into a 2000-mL 3-necked round-bottom flask was placed acetic acid (125 g, 2.08 mol), then added hydrazine hydrate (120 g, 2.40 mol) dropwise with stirring at <10° C. The resulting solution was stirred for 30 min at room temperature, then concentrated in vacuo. The residue was washed with hexane (1×1000 mL) and dried to give hydrazine acetate as a white solid. Into a 100-mL 3-necked round-bottom flask were placed a solution of 3-bromo-N-[(1E)-(dimethylamino)methylene]benzamide (78 g, 306 mmol) in acetic acid (400 mL) and hydrazine acetate (141 g, 1.53 mol). The resulting solution was stirred for 30 min at 95° C. in an oil bath. The resulting mixture was cooled and concentrated in vacuo to remove most of the acetic acid. The residue was diluted with 400 mL of ethyl acetate, and then washed with 2×400 mL of water and 3×400 mL of sodium bicarbonate solution. The organic layer was dried over anhydrous sodium sulfate and concentrated in vacuo to give 3-(3=bromophenyl)-1H-1,2,4-triazole.

LRMS (ESI) calc'd for C8H7BrN3 [M+H]⁺: 224. Found: 224.

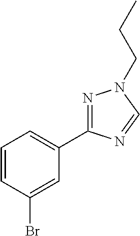

### Step 4. 3-(3-Bromophenyl)-1-propyl-1H-1,2,4-triazole

NaH (60 wt %, 27 mg, 1.1 mmol) was added portionwise to a reaction vessel containing 3-(3-bromophenyl)-1H-1,2,4-triazole (200 mg, 0.893 mmol) in DMF (45 mL). The mixture was allowed to stir at r.t. for 20 minutes followed by the addition 1-iodopropane (0.109 mL, 1.12 mmol). The reaction was stirred overnight at room temperature. Water was added and the products extracted into EtOAc (3×). The combined organic extracts were washed with brine, dried over Na₂SO₄, filtered and concentrated in vacuo. Purification by flash chromatography (MPLC, 0-20% EtOAc-hexanes) gave 3-(3-bromophenyl)-1-propyl-1H-1,2,4-triazole.

LRMS (ESI) calc'd for C11H13BrN3 [M+H]⁺: 266. Found: 266.

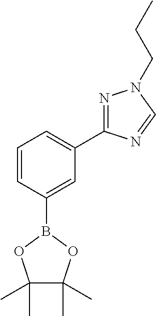

### Step 5. 1-Propyl-3-[-(4,4,5,5-tetramethyl-1,3,2-dioxaborolan-2-yl)phenyl]-1-1H-1,2,4-triazole

3-(3-Bromophenyl)-1-propyl-1H-1,2,4-triazole (134 mg, 0.503 mmol), bis(pinacolato)diboron (192 mg, 0.755 mmol), Pd₂(dba)₃ (9 mg, 10 μmol), XPhos (19 mg, 0.040 mmol), and KOAc (148 mg, 1.51 mmol) were combined in a 5 mL microwave vial. The vial was evacuated and back-filled with N₂ gas (3×) before adding 1,4-dioxane (4.5 mL). The reaction was allowed to stir at 100° C. for 2 hours. The mixture was filtered through Celite and concentrated in vacuo. Purification of the residue by flash chromatography (MPLC, 0-20% EtOAc-hexanes) gave 1-propyl-3-[-(4,4,5,5-tetramethyl-1,3,2-dioxaborolan-2-yl)phenyl]-1-1H-1,2,4-triazole as a white solid.

LRMS (ESI) calc'd for C17H25BN3O2 [M+H]⁺: 314. Found: 314.

The following intermediates were prepared according to Method K following similar procedures described for Intermediate #76, which can be achieved by those of ordinary skill in the art of organic synthesis.

| | | | Exact |
|---|---|---|---|
| | | | Mass |
| Intermediate | Structure | IUPAC Name | [M + H]+ |
| 77 | 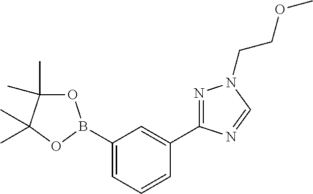 | 1-(2-methoxyethyl)-3-[3- (4,4,5,5-tetramethyl-1,3,2- dioxaborolan-2-yl)phenyl]-1H- 1,2,4-triazole | Calc'd 330, found 330 |
| 78 | 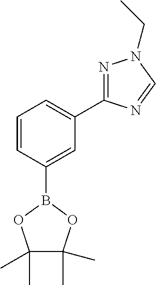 | 1-ethyl-3-[3-(4,4,5,5- tetramethyl-1,3,2- dioxaborolan-2-yl)phenyl]-1H- 1,2,4-triazole | Calc'd 300, found 300 |
| 79 | 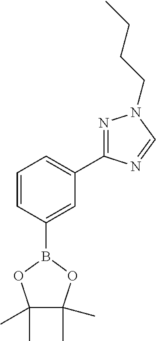 | 1-butyl-3-[3-(4,4,5,5- tetramethyl-1,3,2- dioxaborolan-2-yl)phenyl]-1H- 1,2,4-triazole | Calc'd 328, found 328 |
| 80 | 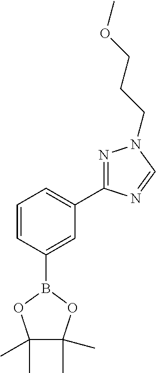 | 1-(3-methoxypropyl)-3-[3- (4,4,5,5-tetramethyl-1,3,2- dioxaborolan-2-yl)phenyl]-1H- 1,2,4-triazole | Calc'd 344, found 344 |
| 81 | 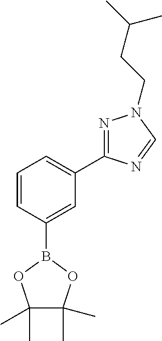 | 1-(3-methylbutyl)-3-[3- (4,4,5,5-tetramethyl-1,3,2- dioxaborolan-2-yl)phenyl]-1H- 1,2,4-triazole | Calc'd 342, found 342 |
| 82 | 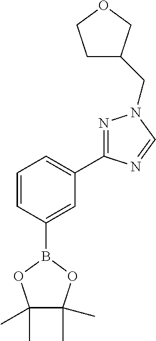 | rac-1-(tetrahydrofuran-3- ylmethyl)-3-[3-(4,4,5,5- tetramethyl-1,3,2- dioxaborolan-2-yl)phenyl]-1H- 1,2,4-triazole | Calc'd 356, found 356 |
| 83 | 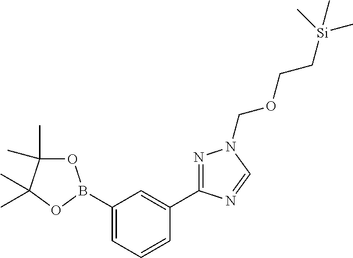 | 3-[3-(4,4,5,5-tetramethyl-1,3,2- dioxaborolan-2-yl)phenyl]-1- {[2- (trimethylsilyl)ethoxy]methyl}- 1H-1,2,4-triazole | Calc'd 402, found 402 |

### Boronic Ester Synthesis Method L

### Intermediate #84

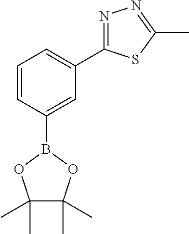

### 2-Methyl-5-[3-(4,4,5,5-tetramethyl-1,3,2-dioxaborolan-2-yl)phenyl]-1,3,4-thiadiazole

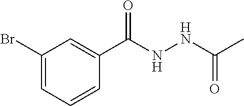

### Step-1. N′-Acetyl-3-bromobenzohydrazide

3-Bromobenzoic acid (525 mg, 2.61 mmol), acetohydrazide (387 mg, 5.22 mmol), EDC (751 mg, 3.92 mmol) and HOBt (600 mg, 3.92 mmol) were combined in DMF (8 mL). The mixture was allowed to stir at 50° C. for 3 hours. The reaction mixture was cooled and diluted with EtOAc (100 mL) and water (75 mL). The organic layer was collected and washed with saturated NaHCO₃ (50 mL). The layers were separated and the organic layer was washed with brine, dried over Na₂SO₄, filtered and allowed to sit. A precipitate formed and was filtered. The resulting filter cake was washed with EtOAc (50 mL) to give N-acetyl-3-bromobenzohydrazide.

LRMS (ESI) calc'd for C9H10BrN2O2 [M+H]⁺: 257. found 257.

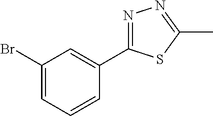

### Step 2. 2-(3-Bromophenyl)-5-methyl-1,3,4-thiadiazole

Lawesson's Reagent (944 mg, 2.334 mmol) was added to N-acetyl-3-bromobenzohydrazide (800 mg, 3.11 mmol) in 1,4-dioxane (10 mL). The mixture was allowed to stir at 100° C. for 1 hour. After cooling to room temperature, silica gel was added and the reaction mixture was concentrated in vacuo. Purification by flash chromatography (MPLC, 0-20% EtOAc-hexanes) gave 2-(3-bromophenyl)-5-methyl-1,3,4-thiadiazole.

LRMS (ESI) calc'd for C9H8BrN2S [M+H]⁺: 255. found 255.

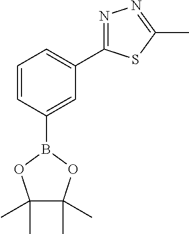

### Step 3. 2-Methyl-5-[3-(4,4,5,5-tetramethyl-1,3,2-dioxaborolan-2-yl)phenyl]-1,3,4-thiadiazole

2-Methyl-5-[3-(4,4,5,5-tetramethyl-1,3,2-dioxaborolan-2-yl)phenyl]-1,3,4-thiadiazole was prepared from 2-(3-bromophenyl)-5-methyl-1,3,4-thiadiazole according to the procedure described for 5-methyl-2-[3-(4,4,5,5-tetramethyl-1,3,2-dioxaborolan-2-yl)phenyl]-1,3-thiazole (Intermediate #46 Step 2).

LRMS (ESI) calc'd for C15H20BN2O2S [M+H]⁺: 303. found 303.

### Boronic Ester Synthesis Method M

### Intermediate #85

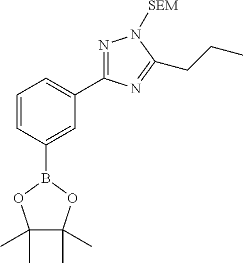

### 5-Propyl-3-[3-(4,4,5,5-tetramethyl-1,3,2-dioxaborolan-2-yl)phenyl]-1-{[2-(trimethylsilyl)ethoxy]methyl}-1H-1,2,4-triazole

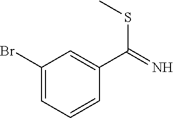

### Step 1. Methyl 3-bromobenzenecarbimidothioate

Iodomethane (0.434 mL, 6.94 mmol) was added to 3-bromobenzenecarbothioamide (1 g, 4.63 mmol) in acetone (4.5 mL) under N₂ gas. The mixture was allowed to stir at ambient temperature for 2 hours. The mixture was filtered, eluting with diethyl ether and the solid was collected to give methyl 3-bromobenzenecarbimidothioate.

LRMS (ESI) calc'd for C8H9BrNS [M+H]⁺: 230. found 230.

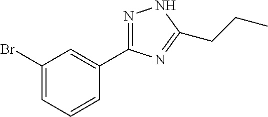

### Step 2. 3-(3-Bromophenyl)-5-propyl-1H-1,2,4-triazole

Butyric acid hydrazide (98 mg, 0.956 mmol) and ammonium acetate (80 mg, 1.043 mmol) were added to methyl 3-bromobenzenecarbimidothioate (200 mg, 0.869 mmol) in EtOH (2 mL). The mixture was allowed to stir at 100° C. for 18 hours. The reaction was cooled, filtered through Celite and concentrated in vacuo. Purification by flash chromatography (MPLC, 10-35% EtOAc-hexanes) gave 3-(3-bromophenyl)-5-propyl-1H-1,2,4-triazole.

LRMS (ESI) calc'd for C11H13BrN3 [M+H]⁺: 266. found 266.

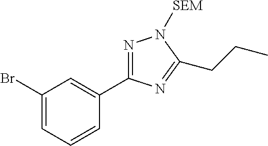

### Step 3. 3-(3-Bromophenyl)-5-propyl-1-{[2-(trimethylsilyl)ethoxy]methyl}-1H-1,2,4-triazole

NaH (60 wt %, 39 mg, 0.986 mmol) was added portionwise to 3-(3-bromophenyl)-5-propyl-1H-1,2,4-triazole (210 mg, 0.789 mmol) in DMF (8 mL). The mixture was allowed to stir at ambient temperature for 1 hour before adding SEM-Cl (0.175 mL, 0.986 mmol). The reaction was allowed to stir at 50° C. for 7 days. Saturated NH₄Cl (50 mL) was added and the products extracted into EtOAc (3×). The combined organic extracts were washed with brine, dried over Na₂SO₄ and concentrated in vacuo. Purification by flash chromatography (MPLC, 0-35% EtOAc-hexanes) gave 3-(3-bromophenyl)-5-propyl-1-{[2-(trimethylsilyl)ethoxy]methyl}-1H-1,2,4-triazole.

LRMS (ESI) calc'd for C17H27BrN30Si [M+H]⁺: 396. found 396.

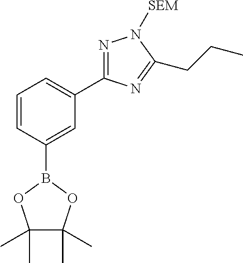

### Step 4. 5-Propyl-3-[3-(4,4,5,5-tetramethyl-1,3,2-dioxaborolan-2-yl)phenyl]-1-{[2-(trimethylsilyl)ethoxy]methyl}-1H-1,2,4-triazole

5-Propyl-3-[3-(4,4,5,5-tetramethyl-1,3,2-dioxaborolan-2-yl)phenyl]-1-{[2-(trimethylsilyl)ethoxy]methyl}-1H-1,2,4-triazole was prepared from 3-(3-bromophenyl)-5-propyl-1-{[2-(trimethylsilyl)ethoxy]methyl}-1H-1,2,4-triazole according to the procedure described for 5-methyl-2-[3-(4,4,5,5-tetramethyl-1,3,2-dioxaborolan-2-yl)phenyl]-1,3-thiazole (Intermediate #46 Step 2).

LRMS (ESI) calc'd for C23H39BN3O3Si [M+H]⁺: 444. found 444.

### Boronic Ester Synthesis Method N

### Intermediate #86

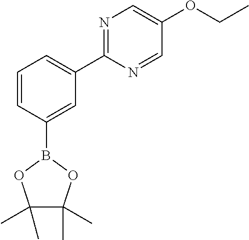

### 5-Ethoxy-2-[3-(4,4,5,5-tetramethyl-1,3,2-dioxaborolan-2-yl)phenyl]pyrimidine

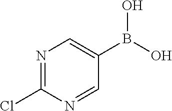

### Step 1. (2-Chloropyrimidin-5-yl)boronic acid

A 5.0 L three necked round bottom flask was charged with 5-bromo-2-chloropyrimidine (200 g, 1034 mmol). THF (900 mL) and toluene (900 mL) were added, followed by triisopropyl borate (294 mL, 1241 mmol). The solution was cooled using a dry ice/acetone bath to −70° C. n-Butyl lithium (496 mL, 1241 mmol) was added dropwise via addition funnel over 1.5 hrs, maintaining the internal temperature at −69° C. to −71° C., forming a clear yellow solution. The reaction mixture was warmed slowly to −45° C. for 1 h-2h, giving a red solution. The reaction was quenched slowly with saturated NH₄Cl (700 mL) at −5° C. or below, resulting in significant precipitate formation. Water (500 mL) was added to dissolve the white-solid. The aqueous phase was separated and acidified with 2 N HCl (−700 mL) to pH ˜1. EtOAc (1.5 L) was added and the biphasic mixture was stirred at r.t. to dissolve all the solid. The aqueous phase was extracted with further portions of EtOAc (2×500 mL). NaCl was added to the aqueous phase until no more would dissolve, then extracted with THF (2×500 mL). All the organic phases were combined and dried over MgSO₄, filtered and concentrated to obtain (2-chloropyrimidin-5-yl)boronic acid as an orange solid.

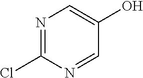

### Step 2. 2-Chloropyrimidin-5-ol

A 2.0 L round bottom flask was charged with (2-chloropyrimidin-5-yl)boronic acid (40.0 g, 253 mmol), THF (440 mL) and water (440 mL). Solid sodium perborate tetrahydrate (117 g, 758 mmol) was added in one portion and the resulting suspension stirred at r.t. for 18 hours. Note: after 10 minutes of solid sodium perborate tetrahydrate addition a small exotherm occurred, from 28° C. to 34° C. over 30 min. The reaction was quenched with saturated NH₄Cl (250 mL) and EtOAc (250 mL) was added. A 10% solution of sodium bisulfite (1 L) was added to the mixture portionwise at 0° C. until no more peroxide was detected by KI-starch paper. (Note: Exotherm occurred during sodium bisulfite addition). The aqueous phase was separated and extracted with further portions of EtOAc (2×250 mL). Solid NaCl was added to the aqueous-phase until no more dissolved then extracted with THF (2×250 mL). All the organic phases were combined and dried-over MgSO₄, filtered and concentrated to obtain a yellow solid. The product-solid was suspended in toluene/heptane (1:1 ratio, 800 mL) and the mixture heated to 50° C. The mixture was cooled to r.t. and the solid collected by filtration and washed with toluene/heptane (1:1 ratio, 250 mL) followed by heptane (150 mL). The product solid was suspended in 15% toluene/DCM (100 mL) at r.t., filtered and washed twice with toluene/DCM (1:1 ratio, 2×50-mL) then with 100% DCM (100 mL) to give 2-chloropyrimidin-5-ol.

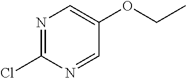

### Step 3. 2-Chloro-5-ethoxypyrimidine

2-Chloropyrimidin-5-ol (13.0 g, 100 mmol) was dissolved in DMF (130 mL) (solution) and K₂CO₃ (27.5 g, 199 mmol) was added (suspension), followed by iodoethane (16.1 mL, 199 mmol). The reaction mixture was stirred at 50° C. for 4 hr and subsequently cooled to ambient temperature and stirred overnight. The reaction mixture was partitioned between EtOAc (650 ml) and 10% aqueous NaCl (650 mL). The organic layer was washed with 10% aqueous NaCl (650 mL). The first aqueous layer was extracted with EtOAc (325 mL). The combined organic layers were dried over Na₂SO₄, filtered, and concentrated in vacuo. The crude mixture was diluted with DCM=to a final volume of 40 mL and purified by flash chromatography (MPLC, 5-40% EtOAc-Hexanes) to provide 2-chloro-5-ethoxypyrimidine as a white solid.

LRMS (ESI) calc'd for C6H8ClNO2 [M+H]⁺: 159. Found: 159.

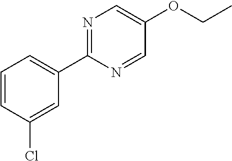

### Step 4. 2-(3-Chlorophenyl)-5-ethoxypyrimidine

2-Chloro-5-ethoxypyrimidine (8.00 g, 50.4 mmol), 3-chlorophenylboronic acid (11.8 g, 76.0 mmol), and PdCl₂(dppf).DCM adduct (4.12 g, 5.04 mmol) were added to a 500 mL round bottom flask, followed by dioxane (80 mL) and 2M Na₂CO₃ (50 mL, 101 mmol). The reaction was purged with argon (subsurface bubbling) for 15 min. A reflux condenser was attached, and the reaction mixture was heated at 100° C. under nitrogen for 14 hrs. The reaction mixture was cooled and diluted with EtOAc (400 mL) and 5% aqueous NH₄Cl (400 mL). The mixture was stirred for 10 min. The biphasic mixture was filtered through Celite and rinsed with EtOAc (2×200 mL). The filtrate was diluted with of 5% aqueous NH₄Cl (400 mL) and the layers were separated. The aqueous layer was extracted with EtOAc (400 mL). The combined organics were dried over Na₂SO₄, filtered and concentrated in vacuo while loading onto silica. The residue was purified by flash chromatography (MPLC, 2-15% EtOAc hexanes) to give 2-(3-chlorophenyl)-5-ethoxypyrimidine as a white solid.

LRMS (ESI) calc'd for Cl2H12ClNO2 [M+H]⁺: 235. Found: 235.

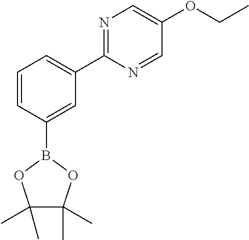

### Step 5. 5-Ethoxy-2-[3-(4,4,5,5-tetramethyl-1,3,2-dioxaborolan-2-yl)phenyl]pyrimidine

XPhos (6.18 g, 12.95 mmol) and Pd2(dba)₃ (2.97 g, 3.24 mmol) were added to a 2 L round bottom flask, followed by de-gassed 1,4-dioxane (446 mL). 2-(3-Chlorophenyl)-5-ethoxypyrimidine (38 g, 162 mmol), bis(pinacolato)diboron (53.5 g, 210 mmol) and KOAc (31.8 g, 324 mmol) were added. The flask was evacuated and back-filled with N₂ before stirring at 95° C. for 6 hours and at r.t. for 18 hours. The reaction mixture was diluted with water and EtOAc. The biphasic mixture was filtered through Celite and the layers separated. The aqueous portion was extracted wuth EtOAc. The combined organic extracts were washed with brine, dried and filtered. 25 wt % Darco was added and the suspension stirred for 1.5 hours. The charcoal was removed by filtering through Celite and the filtrate was concentrated in vacuo. The crude solid was taken-up in hexanes and stirred at 45° C. for 45 minutes. The product solid was collected by filtration, with further crops obtained from the filtrate and combined to give 5-ethoxy-2-[3-(4,4,4,5,5-tetramethyl-1,3,2-dioxaborolan-2-yl)phenyl]pyrimidine.

LRMS (ESI) calc'd for Cl8H23BN2O3 [M+H]⁺: 327. Found: 327.

### Boronic Ester Synthesis Method N

### Intermediate #87

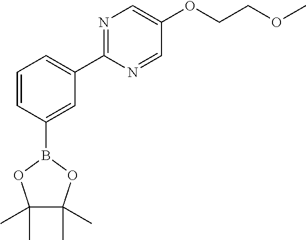

### 5-(2-Methoxyethoxy)-2-[3-(4,4,5,5-tetramethyl-1,3,2-dioxaborolan-2-yl)phenyl]pyrimidine

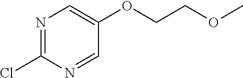

### Step 1. 2-Chloro-5-(2-methoxyethoxy)pyrimidine

To a stirred solution of 2-chloropyrimidin-5-ol (Intermediate #86 Step 2, 17.5 g, 134 mmol) in DMF (90 mL was added-2-bromoethyl methyl ether (17.6 mL, 187 mmol) followed by K₂CO₃ (24.0 g, 174 mmol). The resulting suspension was heated at 60° C. for 18 hr. In order to drive the reaction to completion, additional 2-bromoethyl methyl ether (16.0 mL, 170 mmol) and K₂CO₃ (18.5 g, 134 mmol) were added, and the heating was continued for approximately 8 hr. The reaction mixture was cooled and partitioned between 10% aqueous sodium chloride (250 mL) and EtOAc (500 mL). The layers were separated and the organic layer was washed with 10% aqueous sodium chloride (250 mL). The first aqueous layer was extracted with EtOAc (250 mL). The second aqueous layer was salted with solid NaCl, and extracted with EtOAc (250 mL). The combined organic layers were dried over Na₂SO₄, filtered, and concentrated to a crude oil that was taken up into a minimal amount of DCM and purified by silica gel chromatography to afford 2-chloro-5-(2-methoxyethoxy)pyrimidine as a white solid.

LRMS (ESI) calc'd for C7H10ClN2O2 [M+H]⁺: 189. found 189.

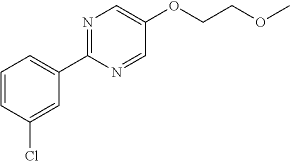

### Step 2. 2-(3-Chlorophenyl)-5-(2-methoxyethoxy)pyrimidine

2-Chloro-5-(2-methoxyethoxy)pyrimidine (15.1 g, 80.0 mmol), (3-chlorophenyl)boronic acid (18.8 g, 120 mmol), and PdCl₂(dppf).DCM (6.55 g, 8.02 mmol) were combined in a 1-liter, 3-neck round bottom flask equipped with a nitrogen inlet, reflux condenser, and mechanical stirrer. To this solid mixture was added dioxane (150 mL) followed by a 2M solution of Na₂CO₃ in water (80 mL, 160 mmol). The resulting mixture was purged with argon (subsurface bubbling) for 15 minutes. The reaction mixture was heated to 100° C. and stirred under nitrogen for approximately 1.5 hr. The reaction mixture was cooled and diluted with EtOAc (400 mL) and 5% aqueous NH₄Cl (400 mL). The resulting biphasic mixture was stirred for approximately 10 min. The biphasic mixture was filtered through Celite and, rinsed with EtOAc (2×200 mL). The filtrate was transferred to a separatory funnel, and the layers were separated. A black solid formed at the phase separation, and this was removed by filtration through a pad of Celite. The layers were separated, and the organic layer was dried over Na₂SO₄, filtered, and concentrated to a crude oil. The resulting oil was taken up into DCM, and purified by column chromatography on silica gel (EtOAc/Hexanes gradient; 2-30%) to afford 2-(3-chlorophenyl)-5-(2-methoxyethoxy)pyrimidine as a white solid.

LRMS (ESI) calc'd for C13H14ClN2O2 [M+H]⁺: 265. found 2.65.

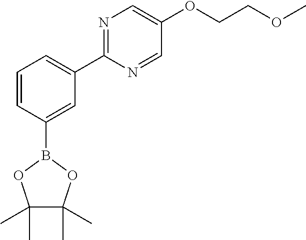

### Step 3. 5-(2-Methoxyethoxy)-2-[3-(4,4,5,5-tetramethyl-1,3,2-dioxaborolan-2-yl)phenyl]pyrimidine

2-(3-Chlorophenyl)-5-(2-methoxyethoxy)pyrimidine (18.0 g, 68.0 mmol), bis(pinacolato)diboron (20.7 g, 82.0 mmol), XPhos (2.59 g, 5.44 mmol), Pd₂(dba)₃ (1.25 g, 1.36 mmol), and KOAc (13.4 g, 136 mmol) were combined in a 1-liter, 3-neck round bottom flask equipped with a mechanical stirrer, reflux condenser, and nitrogen inlet. To the combined solid mixture was added dioxane (180 mL). The resulting suspension was purged with argon (subsurface bubbling) for 15 min. The reaction mixture was heated to 100° C. and stirred under nitrogen for approximately 3.5 hr. The reaction mixture was cooled and diluted with EtOAc (280 mL) and water (70 mL). The resulting mixture was filtered through Celite, rinsing the filter cake with EtOAc (2×50 mL). The filtrate was washed with 75% aqueous sodium bicarbonate (140 mL), followed by 10% aqueous sodium chloride (140 mL). The organic layer was dried over sodium sulfate, filtered, and concentrated in vacuo to a crude solid that was triturated with hexanes (280 mL). The suspension was filtered, rinsing the filter cake with hexanes (2×50 mL) to afford 5-(2-methoxyethoxy)-2-[3-(4,4,5,5-tetramethyl-1,3,2-dioxaborolan-2-yl)phenyl]pyrimidine as white solid.

LRMS (ESI) calc'd for C19H26BN2O4 [M+H]⁺: 357. found 357.

The following intermediates were prepared according to Method N following similar procedures described for Intermediates #86 and 87, which can be achieved by those of ordinary skill in the art of organic synthesis.

| | | | Exact Mass |
|---|---|---|---|
| Intermediate | Structure | IUPAC Name | [M + H]+ |
| 88 | 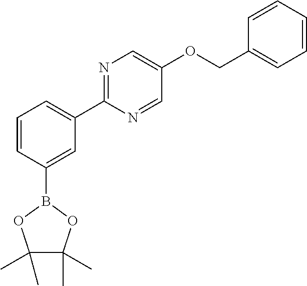 | 5-(benzyloxy)-2-[3- (4,4,5,5-tetramethyl- 1,3,2-dioxaborolan-2- yl)phenyl]pyrimidine | Calc'd 389, Found 389 |
| 89 | 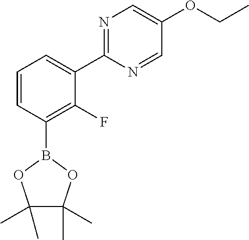 | 5-ethoxy-2-[2-fluoro- 3-(4,4,5,5- tetramethyl-1,3,2- dioxaborolan-2- yl)phenyl]pyrimidine | Calc'd 263, Found 263 (boronic acid) |
| 90 | 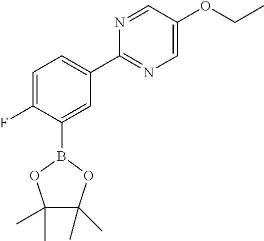 | 5-ethoxy-2-[4-fluoro- 3-(4,4,5,5- tetramethyl-1,3,2- dioxaborolan-2- yl)phenyl]pyrimidine | Calc'd 345, Found 345 |
| 91 | 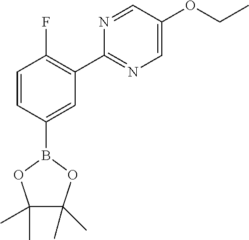 | 5-ethoxy-2-[2-fluoro- 5-(4,4,5,5- tetramethyl-1,3,2- dioxaborolan-2- yl)phenyl]pyrimidine | Calc'd 345, Found 345 |
| 92 | 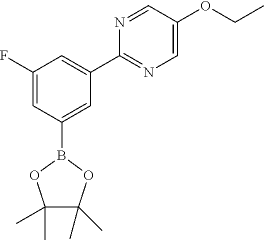 | 5-ethoxy-2-[3-fluoro- 5-(4,4,5,5- tetramethyl-1,3,2- dioxaborolan-2- yl)phenyl]pyrimidine | Calc'd 345, Found 345 |
| 93 | 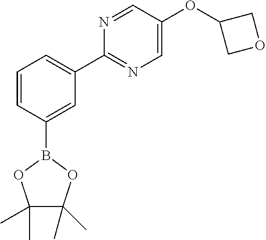 | 5-(oxetan-3-yloxy)-2- [3-(4,4,5,5- tetramethyl-1,3,2- dioxaborolan-2- yl)phenyl]pyrimidine | Calc'd 355, Found 355 |

### Boronic Ester Synthesis Method N

### Intermediate #94

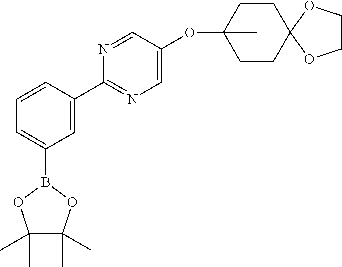

### 5-(1,4-Dioxaspiro[4.5]dec-8-yloxy)-2-[3-(4,4,5,5-tetramethyl-1,3,2-dioxaborolan-2-yl)phenyl]pyrimidine

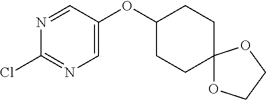

### Step 1,2-Chloro-5-(1,4-dioxaspiro[4.5]dec-8-yloxy)pyrimidine

2-Chloropyrimidin-5-ol (Intermediate #86 Step 2, 200 mg, 1.5 mmol) was dissolved in THF (5 mL) and triphenylphosphine (600 mg, 23 mmol) and 1,4-dioxa-spiro[4.5]decan-8-ol (365 mg, 2.30 mmol) was added, followed by DIAD (0.45 mL, 2.3 mmol). The reaction mixture was stirred at ambient temperature overnight. The reaction mixture was diluted with EtOAc, washed with saturated NaHCO₃, and the aqueous phase was extracted with EtOAc. The combined organic extracts were dried over Na₂SO₄, filtered, dry loaded onto silica gel and the crude residue was purified by flash chromatography-(MPLC, 2-20% DCM-hexane followed by 5-60% EtOAc-hexane) to give a crude residue which was further purified by reverse phase preparative HPLC (0-80% MeCN—H₂O, 0.05% TFA). Fractions containing the pure compound were collected and the free base was liberated by an EtOAc extraction and sat. NaHCO₃ wash to give 2-chloro-5-(1,4-dioxaspiro[4.5]dec-8-yloxy)pyrimidine as a solid.

LRMS (ESI) calc'd for C12H16ClN2O3 [M+H]⁺: 271. Found: 271.

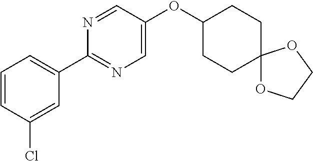

### Step 2. 2-(3-Chlorophenyl)-5-(1,4-dioxaspiro[4,5]dec-8-yloxy)pyrimidine

2-(3-Chlorophenyl)-5-(1,4-dioxaspiro[4.5]dec-8-yloxy)pyrimidine was prepared from 2-chloro-5-(1,4-dioxaspiro[4.5]dec-8-yloxy)pyrimidine according to the procedure described for 2-(3-chlorophenyl)-5-ethoxypyrimidine (Intermediate #86 Step 4).

LRMS (ESI) calc'd for C18H20ClN2O3 [M+H]⁺: 347. Found: 347.

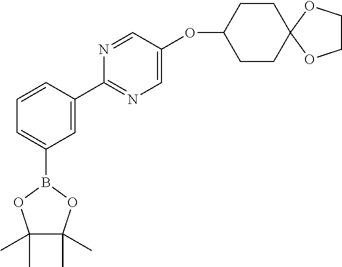

### Step 3. 5-(1,4-Dioxaspiro 4.51 dec-8-yloxy)-2-[3-(4,4,5,5-tetramethyl-1,3,2-dioxaborolan-2-yl)phenyl]pyrimidine

5-(1,4-Dioxaspiro[4.5]dec-8-yloxy)-2-[3-(4,4,5,5-tetramethyl-1,3,2-dioxaborolan-2-yl)phenyl]pyrimidine was prepared from 2-(3-chlorophenyl)-5-(1,4-dioxaspiro[4.5]dec-8-yloxy)pyrimidine according to the procedure described for 5-ethoxy-2-[3-(4,4,5,5-tetramethyl-1,3,2-dioxaborolan-2-yl)phenyl]pyrimidine (Intermediate #86 Step 5).

LRMS (ESI)-calc'd for C24H32BN2O5 [M+H]⁺: 439. Found: 439.

### Boronic Ester Synthesis Method O

### Intermediate #95

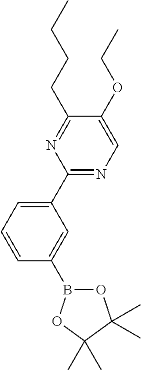

### 4-Butyl-5-ethoxy-2-[3-(4,4,5,5-tetramethyl-1,3,2-dioxaborolan-2-yl)phenyl]pyrimidine

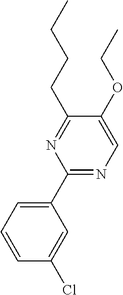

### Step 1. 4-Buty-2-(3-chlorophenyl)-5-ethoxypyrimidine

To a solution of 2-(3-chlorophenyl)-5-ethoxypyrimidine (Intermediate #86 Step 4, 300 mg, 1.28 mmol) was taken up in THF (6.5 mL) and cooled to −78° C., forming a thick suspension. n-BuLi (2.5 M in hexanes, 1.02 mL, 2.56 mmol) was added dropwise and the mixture stirred for 1 hour. MeOH (2.4 mL) was added and the mixture was warmed to r.t. A solution of CAN in H₂O (2 mL) was added and the reaction was allowed to stir overnight at r.t. Saturated NH₄Cl was added and the products extracted into EtOAc (X2). The combined organic layers were washed with brine, dried over Na₂SO₄, filtered and concentrated in vacuo. The residue was purified by flash chromatography (MPLC, 20-80% EtOAc-hexanes) to afford 4-butyl-2-(3-chlorophenyl)-5-ethoxypyrimidine.

LRMS (ESI) calc'd for C16H20ClN2O [M+H]⁺: 291. Found: 291.

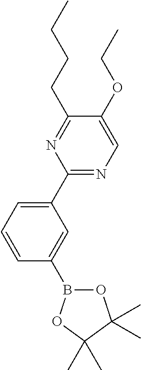

### Step 2. 4-Butyl-5-ethoxy-2-[3-(4,4,5,5-tetramethyl-1,32-dioxaborolan-2-yl)phenyl]pyrimidine

4-Butyl-2-(3-chlorophenyl)-5-ethoxypyrimidine (500 mg, 0.51 mmol), bis(pinacolato)diboron (157 mg, 0.619 mmol), Pd₂(dba)₃ (9.5 mg, 0.010 mmol), XPhos (20 mg, 0.041 mmol), and KOAc (101 mg, 1.03 mmol) were added to a microwave vial followed by the addition of dioxane (1.8 mL). The resulting suspension was purged with argon (subsurface bubbling) for 10 min. The vial was stirred at 100° C. for approximately 4 hrs. The reaction mixture was cooled, then poured into 10 mL of EtOAc. The reaction mixture was stirred at ambient for 10 min, filtered through Celite and rinsed with EtOAc (2×10 mL) to remove the salts. The filtrate was concentrated to afford 4-butyl-5-ethoxy-2-[3-(4,4,5,5-tetramethyl-1,3,2-dioxaborolan-2-yl)phenyl]pyrimidine as a crude orange oil that solidified on high vacuum.

LRMS (ESI) calc'd for C22H32BN2O3 [M+H]⁺: 383. Found: 383.

The following intermediates were prepared according to Method O following similar procedures described for Intermediate #95, which can be achieved by those of ordinary skill in the art of organic synthesis.

| Inter- | | | |
|---|---|---|---|
| medi- | | | Exact Mass |
| ate | Structure | IUPAC Name | [M + H]+ |
| 96 | 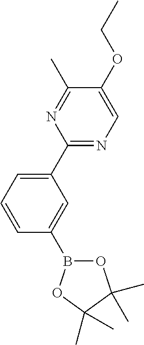 | 5-ethoxy-4-methyl- 2-[3-(4,4,5,5- tetramethyl-1,3,2- dioxaborolan-2- yl)phenyl]pyrimidine | Calc'd 341, Found 341 |
| 97 | 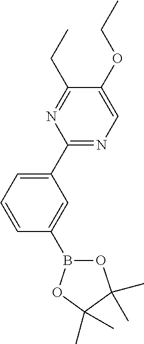 | 5-ethoxy-4-ethyl-2- [3-(4,4,5,5- tetramethyl-1,3,2- dioxaborolan-2- yl)phenyl]pyrimidine | Calc'd 273, Found 273 (boronic acid) |

### Boronic Ester Synthesis Method P

### Intermediate #98

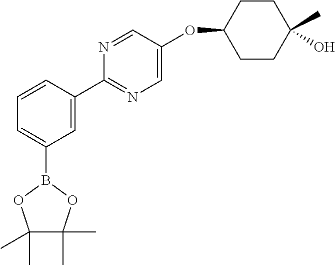

### trans-1-Methyl-4-({2-[3-(4,4,5,5-tetramethyl-1,3,2-dioxaborolan-2-yl)phenyl]pyrimidin-5-yl}oxy)cyclohexanol

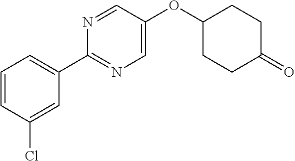

### Step 1. 4-{[2-(3-Chlorophenyl)pyrimidin-5-yl]oxy}cyclohexanone

2-Chloro-5-(1,4-dioxaspiro[4.5]dec-8-yloxy)pyrimidine (Intermediate #94 Step 2, 515 mg, 1.5 mmol) was dissolved in THF (10 mL) and 6 N HCl (5 mL, 30 mmol) was added. The reaction mixture was stirred at r.t. for 1 hr. The reaction mixture was diluted with EtOAc, washed with saturated NaHCO₃, and the aqueous phase was extracted with EtOAc. The combined organic extracts were dried over Na₂SO₄, filtered, dry loaded onto silica gel and the crude residue was purified by flash chromatography (MPLC, 5-50% EtOAc-hexane) to give 4-{[2-(3-chlorophenyl)pyrimidin-5-yl]oxy}cyclohexanone as a white solid.

LRMS (ESI) calc'd for C16H16ClN2O2 [M+H]⁺: 303. Found: 303.

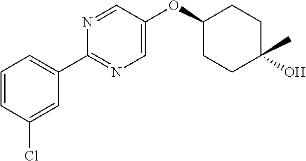

### Step 2. trans-4-{[2-(3-Chlorophenyl)pyrimidin-5-yl]oxy}-1-methylcyclohexanol

4-{[2-(3-Chlorophenyl)pyrimidin-5-yl]oxy}cyclohexanone (300 mg, 1.0 mmol) was dissolved in THF (5 mL) and cooled to −78° C. Methylmagnesium bromide (3 M in diethyl ether, 0.5 mL, 1.50 mmol) was added slowly. The reaction mixture was stirred for 1.5 hrs. The reaction mixture was diluted with EtOAc, washed with saturated NH₄Cl and the aqueous phase was extracted with EtOAc. The combined organic extracts were dried-over Na₂SO₄, filtered, dry loaded onto silica gel and the crude residue was purified by flash chromatography (MPLC, 5-50% EtOAc-hexane) to give trans-4-{[2-(3-chlorophenyl)pyrimidin-5-yl]oxy}-1-methylcyclohexanol as a gum.

LRMS (ESI) calc'd for C17H20ClN2O2 [M+H]⁺: 319. Found: 319.

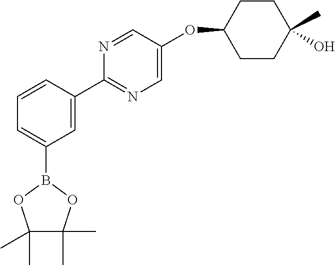

### Step 3. trans-1-Methyl-4-({2-[3-(4,4,5,5-tetramethyl-1,3,2-dioxaborolan-2-yl)phenyl]pyrimidin-5-yl}oxy)cyclohexanol

trans-1-Methyl-4-({2-[3-(4,4,5,5-tetramethyl-1,3,2-dioxaborolan-2-yl)phenyl]pyrimidin-5-yl}oxy)cyclohexanol was prepared from trans-4-{[2-(3-chlorophenyl)pyrimidin-5-yl]oxy}-1-methylcyclohexanol according to the procedure described for 5-ethoxy-2-[3-(4,4,5,5-tetramethyl-1,3,2-dioxaborolan-2-yl)phenyl]pyrimidine (Intermediate #86 Step 5).

LRMS (ESI) calc'd for C23H32BN2O4 [M+H]⁺: 411. Found: 411.

### Boronic Ester Synthesis Method Q

### Intermediate #99

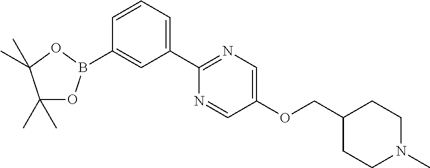

### 5-[(1-Methylpiperidin-4-yl)methoxy]-2-[3-(4,4,5,5-tetramethyl-1,3,2-dioxaborolan-2-yl)phenyl]pyrimidine

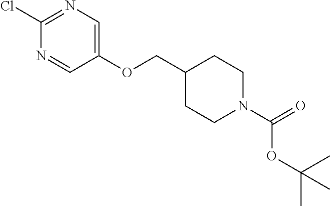

### Step 1. tert-Butyl 4-{[(2-chloropyrimidin-5-yl)oxy]methyl}piperidine-1-carboxylate

To a pressure vial equipped with a stir bar was added tert-butyl 4-(bromomethyl)piperidine-1-carboxylate (4.95 g, 17.8 mmol), 2-chloropyrimidin-5-ol (Intermediate #86 Step 2, 2.3 g, 17.8 mmol) and DMF (59 mL). Sodium hydride (60 wt %, 0.47 g, 19.6 mmol) was added and the vial was sealed and heated to 60° C. for 24 hours. The crude reaction mixture was diluted with ethyl acetate and filtered through a column pre-packed with Celite. The filtrate was concentrated in vacuo and the residue purified by flash chromatography (MPLC, 0-50% EtOAc-hexanes) to give tert-butyl 4-{[(2-chloropyrimidin-5-yl)oxy]methyl}piperidine-1-carboxylate.

LRMS (ESI) calc'd for C11H15ClN3O3 [M+H]⁺: 272. Found: 272 (carbamic acid).

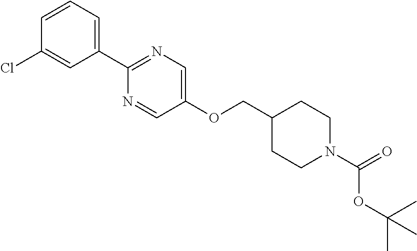

### Step 2. tert-Butyl 4-({[2-(3-chlorophenyl)pyrimidin-5-yl]oxy}methyl)piperidine-1-carboxylate

To a microwave vial equipped with a stir bar was added tert-butyl 4-{[(2-chloropyrimidin-5-yl)oxy]methyl}piperidine-1-carboxylate (602 mg, 1.84 mmol), potassium carbonate (508 mg, 3.67 mmol), (3-chlorophenyl)boronic acid (431 mg, 2.75 mmol), bis(triphenylphosphine)palladium(II) chloride (26 mg, 0.037 mmol) and EtOH (6.1 mL)/toluene (3.1 mL). The vial was purged with nitrogen for 5 minutes and was then sealed and heated in the microwave to 80° C. for 30 minutes. The crude reaction mixture was filtered through Celite and the filtrate was concentrated in vacuo. The residue was purified by flash chromatography (MPLC, 0-50% EtOAc-hexanes) to give tert-butyl 4-({[2-(3-chlorophenyl)pyrimidin-5-yl]oxy}methyl)piperidine-1-carboxylate.

LRMS (ESI) calc'd for C17H19ClN3O3 [M+H]⁺: 348. Found: 348.

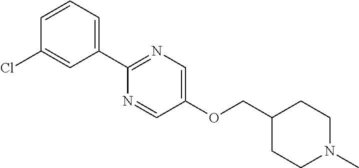

### Step 3. 2-(3-Chlorophenyl)-5-(1-methylpiperidin-4-yl)methoxyl pyrimidine

To a round bottom flask equipped with a stir bar was added tert-butyl 4-({[2-(3-chlorophenyl)pyrimidin-5-yl]oxy}methyl)piperidine-1-carboxylate (574 mg, 1.42 mmol) and THF (7.1 mL). The reaction mixture was cooled to 0° C. DIBAL-H (1 M in THF, 4.3 mL, 4.3 mmol) was added dropwise and the reaction was warmed to room temperature and stirred for 22 hours. Additional DIBAL-H (1 M in THF, 4.3 mL, 4.3 mmol) was added and the reaction reached full conversion after 35 minutes. The reaction mixture was cooled to 0° C. and saturated sodium sulfate decahydrate was added in excess to quench the DIBAL-H. The crude reaction mixture was filtered and the filtrate concentrated in vacuo while loading onto silica. The residue was purified by flash chromatography (MPLC, 0-15% MeOH-DCM) to give 2-(3-chlorophenyl)-5-[(1-methylpiperidin-4-yl)methoxy]pyrimidine.

LRMS (ESI) calc'd for C17H21ClN30 [M+H]⁺: 318. Found: 318.

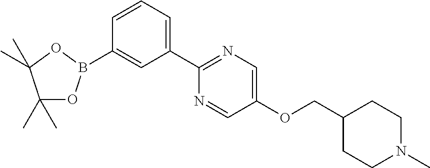

### Step 4. 5-[(1-Methylpiperidin-4-yl)methoxy]-2-[3-(4,4,5,5-tetramethyl-1,3,2-dioxaborolan-2-yl)phenyl]pyrimidine

5-[(1-Methylpiperidin-4-yl)methoxy]-2-[3-(4,4,5,5-tetramethyl-1,3,2-dioxaborolan-2-yl)phenyl]pyrimidine was prepared from 2-(3-chlorophenyl)-5-[(1-methylpiperidin-4-yl)methoxy]pyrimidine according to the procedure described for 5-ethoxy-2-[3-(4,4,5,5-tetramethyl-1,3,2-dioxaborolan-2-yl)phenyl]pyrimidine (Intermediate #86 Step 5).

LRMS (ESI) calc'd for C23H33BN3O3 [M+H]⁺: 410. Found: 410.

### Boronic Ester Synthesis Method R

### Intermediate #100

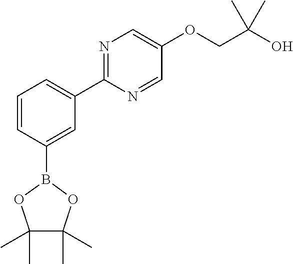

### 2-Methyl-1-({2-[3-(4,4,5,5-tetramethyl-1,3,2-dioxaborolan-2-yl)phenyl]pyrimidin-5-yl}oxy)propan-2-ol

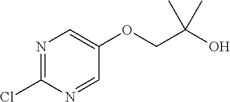

### Step 1. 1-[(2-Chloropyrimidin-5-yl)oxy]-2-methylpropan-2-ol

2-Chloropyrimidin-5-ol (Intermediate #86 Step 2, 5.0 g, 38.3 mmol), K₂CO₃ (10.6 g, 77 mmol) and 2,2-dimethyloxirane (6.81 mL, 77 mmol) were stirred in DMF (50 mL) at 50° C. for 4 hours followed by 65° C. for 3 days. Water was added, followed by saturated NH₄Cl and EtOAc. The resulting emulsion was filtered through Celite. The organic phase was separated and the aqueous portion extracted again with EtOAc. The combined organic extracts were washed with brine, dried over MgSO₄ and concentrated in vacuo. Purification of the residue by flash chromatography (MPLC, 12-100% EtOAc-hexanes) gave 1-[(2-chloropyrimidin-5-yl)oxy]-2-methylpropan-2-ol as a white solid.

LRMS (ESI) calc'd for C8H12ClN2O2 [M+H]⁺: 203. found 203.

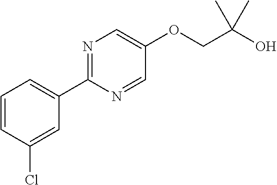

### Step 2. 1-[2-(3-Chlorophenyl)pyrimidin-5-yl)oxy]-2-methylpropan-2-ol

1-{[2-(3-Chlorophenyl)pyrimidin-5-yl]oxy}-2-methylpropan-2-ol was prepared from 1-[(2-chloropyrimidin-5-yl)oxy]-2-methylpropan-2-ol according to the procedure described for 2-(3-chlorophenyl)-5-ethoxypyrimidine (Intermediate #86, Step 4).

LRMS (ESI) calc'd for C14H16ClN2O2 [M+H]⁺: 279. found 279.

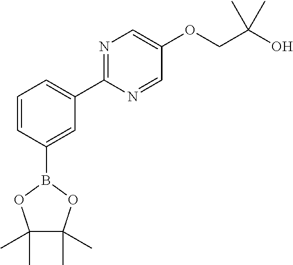

### Step 3. 2-Methyl-1-({2-[3-(4,4,5,5-tetramethyl-1,3,2-dioxaborolan-2-yl)phenyl]pyrimidin-5-yl}oxy)propan-2-ol

2-Methyl-1-({2-[3-(4,4,5,5-tetramethyl-1,3,2-dioxaborolan-2-yl)phenyl]pyrimidin-5-yl}oxy)propan-2-ol was prepared from 1-{[2-(3-chlorophenyl)pyrimidin-5-yl]oxy}-2-methylpropan-2-ol according to the procedure described for 5-ethoxy-2-[3-(4,4,5,5-tetramethyl-1,3,2-dioxaborolan-2-yl)phenyl]pyrimidine (Intermediate #86 Step 5).

LRMS (ESI) calc'd for C20H28BN2O4 [M+H]⁺: 371. found-371.

### Boronic Ester Synthesis Method S

### Intermediate #101

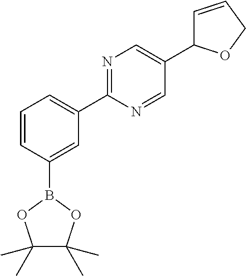

### 5-(2,5-Dihydrofuran-2-yl)-2-[3-(4,4,5,5-tetramethyl-1,3,2-dioxaborolan-2-yl)phenyl]pyrimidine

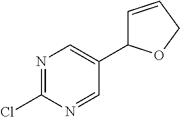

### Step 1. 2-Chloro-5-(2,5-dihydrofuran-2-yl)pyrimidine

To a stirred suspension of 5-bromo-2-chloropyrimidine (0.500 g, 2.58 mmol), tri-t-butylphosphoniumtetrafluoroborate (0.075g, 0.26 mmol) and Pd₂(dba)₃ (0.118 g, 0.129 mmol) in 1,4-dioxane (5 mL) was added 2,3-dihydrofuran (0.1-96 mL, 2.58 mmol) and N-methyldicyclohexylamine (1.11 mL, 5.17 mmol). The reaction mixture was purged with argon (subsurface bubbling) for 5 min. The reaction was heated to 80° C. and stirred for 30 min. The reaction mixture was cooled, diluted with EtOAc and silica gel was added. The resulting mixture was concentrated to a crude solid that was purified by flash chromatography (MPLC, 2-20% EtOAc-hexanes) to afford 2-chloro-5-(2,5-dihydrofuran-2-yl)pyrimidine.

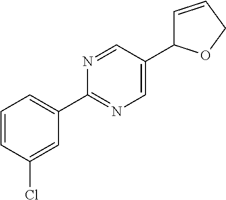

### Step 2. 2-(3-Chlorophenyl)-5-(2,5-dihydrofuran-2-yl)pyrimidine

2-(3-Chlorophenyl)-5-(2,5-dihydrofuran-2-yl)pyrimidine was prepared from 2-chloro-5-(2,5-dihydrofuran-2-yl)pyrimidine according to the procedure described for 2-(3-chlorophenyl)-5-ethoxypyrimidine (Intermediate #86, Step 4).

LRMS (ESI) calc'd for C14H12ClN2O [M+H]⁺: 259. Found: 259.

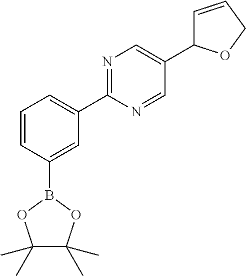

### Step 3. 5-(2,5-Dihydrofuran-2-yl)-2-[3-(4,4,5,5-tetramethyl-1,3,2-dioxaborolan-2-yl)phenyl]pyrimidine

5-(2,5-Dihydrofuran-2-yl)-2-[3-(4,4,5,5-tetramethyl-1,3,2-dioxaborolan-2-yl)phenyl]pyrimidine was prepared from 2-(3-chlorophenyl)-5-(2,5-dihydrofuran-2-yl)pyrimidine according to the procedure described for 5-ethoxy-2-[3-(4,4,5,5-tetramethyl-1,3,2-dioxaborolan-2-yl)phenyl]pyrimidine (Intermediate #86 Step 5).

LC/MS (ESI) calc'd for C20H24BN2O3 [M+H]⁺, 351. found 351.

### Boronic Ester Synthesis Method S

### Intermediate #102

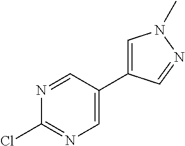

### Step 1. 2-Chloro-5-(1-methyl-1H-pyrazol-4-yl)pyrimidine

A microwave vial was charged with 5-bromo-2-chloropyrimidine (4.0 g, 20.0 mmol), 1-methyl-4-(4,4,5,5-tetramethyl-1,3,2-dioxaborolan-2-yl)-1H-pyrazole (4.3 g, 20.0 mmol), Cs₂CO₃ (20.2 g, 62.0 mmol), PdCl₂(dppf).DCM (0.84 g, 1.0 mmol), 1,4-dioxane (86 mL) and water (17 mL). The reaction mixture was degassed by bubbling N₂ through before heating to 100° C. for 24 hours. Room temperature was attained and the reaction mixture-filtered through Celite. The filtrate was evaporated onto silica and the residue purified by flash chromatography (MPLC, 20-100% EtOAc-hexanes, followed by 0-20% MeOH-DCM) to give 2-chloro-5-(1-methyl-1H-pyrazol-4-yl)pyrimidine.

LRMS (ESI) calc'd for C8H8ClN4 [M+H]⁺: 195. Found: 195.

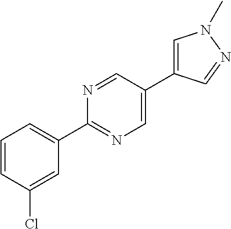

### Step 2. 2-(3-Chlorophenyl)-5-(1-methyl-1H-pyrazol-4-yl)pyrimidine

2-(3-Chlorophenyl)-5-(1-methyl-1H-pyrazol-4-yl)pyrimidine was prepared from 2-chloro-5-(1-methyl-1H-pyrazol-4-yl)pyrimidine according to the procedure described for 2-(3-chlorophenyl)-5-ethoxypyriminde (Intermediate #86, Step 4).

LRMS (ESI) calc'd for C14H12ClN4 [M+H]⁺: 271. Found: 271.

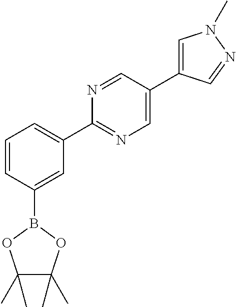

### Step 3. 5-(1-Methyl-1H-pyrazol-4-yl)-2-[3-(4,4,5,5-tetramethyl-1,3,2-dioxaborolan-2-yl)phenyl]pyrimidine

5-(1-Methyl-1H-pyrazol-4-yl)-2-[3-(4,4,5,5-tetramethyl-1,3,2-dioxaborolan-2-yl)phenyl]pyrimidine was prepared from 2-(3-chlorophenyl)-5-(1-methyl-1H-pyrazol-4-yl)pyrimidine according to the procedure described for 5-ethoxy-2-[3-(4,4,5,5-tetramethyl-1,3,2-dioxaborolan-2-yl)phenyl]pyrimidine (Intermediate #86 Step 5).

LRMS (ESI) calc'd for C20H24N4BO2 [M+H]⁺: 363. Found: 363.

### Boronic Ester Synthesis Method S

### Intermediate #103

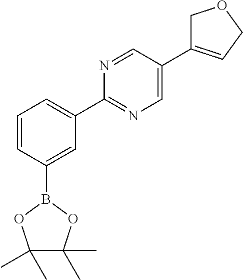

### 5-(2,5-Dihydrofuran-3-yl)-2-[3-(4,4,5,5-tetramethyl-1,3,2-dioxaborolan-2-yl)phenyl]pyrimidine

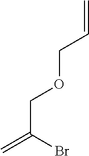

### Step 1. 2-Bromo-3-(prop-2-en-1-yloxy)prop-1-ene

2-Bromoprop-2-en-1-ol (10.0 g, 73.0 mmol) and 3-bromoprop-1-ene (12.4 mL, 146 mmol) were added to a flask followed by DMF (200 mL). The mixture was cooled to 0° C. and NaH (60 wt %, 3.2 g, 80.0 mmol) was added cautiously. After total addition of NaH, the cooling bath was removed and the reaction was allowed to stir at r.t. for 15 minutes. The reaction was quenched with 5% ammonium chloride and extracted with Et₂O (x2). The combined organic extracts were dried over MgSO₄, filtered and concentrated to a crude oil. The oil was taken up in a minimal amount of DCM and was purified by flash chromatography (MPLC, 2-20% EtOAc-hexanes) to afford 2-bromo-3-(prop-2-en-1-yloxy)prop-1-ene as a colorless oil.

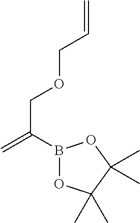

### Step 2. 4,4,5,5-Tetramethyl-2-[3-(prop-2-en-1-yloxy)prop-1-en-2-yl]-1,3,2-dioxaborolane

To a 25 mL round bottom flask was added 2-bromo-3-(prop-2-en-1-yloxy)prop-1-ene (500 mg, 2.82 mmol) and Et₂O (5.4 mL) under nitrogen. The mixture was cooled to −78° C. before t-BuLi (1.7 M, 2.49 mL, 4.24 mmol) was added dropwise. After 30 min, 2-isopropoxy-4,4,5,5-tetramethyl-1,3,2-dioxaborolane (1.15 mL, 5.65 mmol) was added by syringe. The reaction was allowed to stir at −78° C. for about 1 hr. The cooling bath was removed and the reaction was stirred at r.t. for another hour. A viscous white solution formed and the reaction was then quenched by the addition of water and additional Et₂O. The aqueous phase was adjusted to a pH of 7 with 2N HCl. The layers were separated, and the aqueous solution was extracted with additional Et₂O. The combined organic extracts were washed with brine, dried over MgSO₄, filtered and concentrated in vacuo. The residual oil was purified by flash chromatography (MPLC, 2-20% EtOAc-hexanes) to afford 4,4,5,5-tetramethyl-2-[3-(prop-2-en-1-yloxy)prop-1-en-2-yl]-1,3,2-dioxaborolane as a colorless oil.

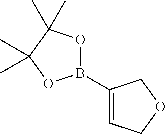

### Step-3. 2-(2,5-Dihydrofuran-3-yl)-4,4,5,5-tetramethyl-1,3,2-dioxaborolane

2-(2,5-Dihydrofuran-3-yl)-4,4,5,5-tetramethyl-1,3,2-dioxaborolane was prepared according to the method described by Renaud, J.; Ouellet, S., J. Am. Chem. Soc., 1998, 120, 7995: A solution of 4,4,5,5-tetramethyl-2-[3-(prop-2-en-1-yloxy)prop-1-en-2-yl]-1,3,2-dioxaborolane (220 mg, 0.982 mmol) in DCM (19 mL) was degassed with N₂ for 5 minutes. Grubbs II catalyst (42 mg, 0.049 mmol) was added to the reaction and N₂ was bubbled through the reaction for another 5 minutes. The reaction was then stirred for 18 hours at r.t. The reaction was filtered through Celite and washed with DCM. The filtrate was concentrated in vacuo and the residue was then redissolved in DCM (20 mL). Scavenger Siliabond DMT (Silicycle, 9g, 4.91 mmol) was added and reaction was allowed to stir for 18 hours. The mixture was filtered through Celite and washed with DCM. The filtrate was concentrated in vacuo to afford 2-(2,5-dihydrofuran-3-yl)-4,4,5,5-tetramethyl-1,3,2-dioxaborolane.

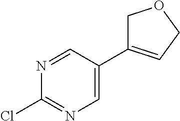

### Step 4. 2-Chloro-5-(2,5-dihydrofuran-3-yl)pyrimidine

To a vial was added 5-bromo-2-chloropyrimidine (100 mg, 0.517 mmol), 2-(2,5-dihydrofuran-3-yl)-4,4,5,5-tetramethyl-1,3,2-dioxaborolane (122 mg, 0.620 mmol), S-Phos (21 mg, 0.057 mmol), K₃PO₄ (329 mg, 1.55 mmol) and PdOAc₂ (5.8 mg, 0.026 mmol). THF (2.3 mL) and water (0.3 mL) were added and nitrogen was bubbled through the mixture for about 5 minutes. The reaction was stirred at 60° C. for about 3 hrs. Saturated NH₄Cl was added and the products extracted into ethyl acetate. The organic layer was dried and filtered. The filtrate was concentrated in vacuo and the residue purified by flash chromatography (MPLC, 40% EtOAc-hexanes) to afford 2-chloro-5-(2,5-dihydrofuran-3-yl)pyrimidine.

LRMS (ESI) calc'd for C8H8ClN20 [M+H]⁺: 183. Found: 183.

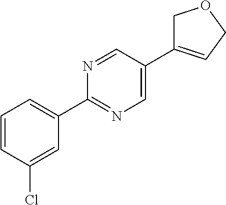

### Step 5. 2-(3-Chlorophenyl)-5-(2,5-dihydrofuran-3-yl)pyrimidine

2-(3-Chlorophenyl)-5-(2,5-dihydrofuran-3-yl)pyrimidine was prepared from 2-chloro-5-(2,5-dihydrofuran-3-yl)pyrimidine according to the procedure described for 2-(3-chlorophenyl)-5-ethoxypyriminde (Intermediate #86, Step 4).

LRMS (ESI) calc'd for C14H12ClN20 [M+H]⁺: 259. Found: 259.

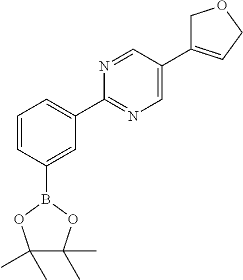

### Step 6. 5-(2,5-Dihydrofuran-3-yl)-2-[3-(4,4,5,5-tetramethyl-1,3,2-dioxaborolan-2-yl)phenyl]pyrimidine

5-(2,5-Dihydrofuran-3-yl)-2-[3-(4,4,5,5-tetramethyl-1,3,2-dioxaborolan-2-yl)phenyl]pyrimidine was prepared from 2-(3-chlorophenyl)-5-(2,5-dihydrofuran-3-yl)pyrimidine according to the procedure described for 5-ethoxy-2-[3-(4,4,5,5-tetramethyl-1,3,2-dioxaborolan-2-yl)phenyl]pyrimidine (Intermediate #86 Step 5).

LRMS (ESI) calc'd for C20H24BN2O3 [M+H]⁺: 351. Found: 351.

### Boronic Ester Synthesis Method S

### Intermediate #104

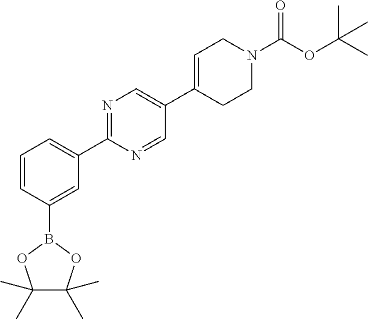

### tert-Butyl 4-{2-[3-(4,4,5,5-tetramethyl-1,3,2-dioxaborolan-2-yl)phenyl]pyrimidin-5-yl}-3,6-dihydropyridine-1(2H)-carboxylate

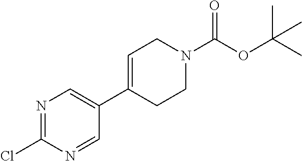

### Step 1. tert-Butyl 4-(2-chloropyrimidin-5-yl)-3,6-dihydropyridine-1(2H)-carboxylate

A stirred suspension of tert-butyl 4-(4,4,5,5-tetramethyl-1,3,2-dioxaborolan-2-yl)-3,6-dihydropyridine-1(2H)-carboxylate (1.92 g, 6.20 mmol), 5-bromo-2-chloropyrimidine (1.00 g, 5.17 mmol), PdCl₂(dppf).DCM adduct (0.211 g, 0.258 mmol), and cesium carbonate (3.37 g, 10.3 mmol) in 1,4-dioxane (13 mL) and water (2.6 mL) was purged with argon (subsurface bubbling) for 5 min. The reaction mixture was stirred at 60° C. for 2.5 h. The reaction mixture was cooled, diluted with EtOAc, and filtered through Celite. Silica gel was added to the filtrate, and the resulting mixture was concentrated to a crude solid that was purified by flash chromatography (MPLC, 5-40% EtOAc-hexanes) to afford tert-butyl 4-(2-chloropyrimidin-5-yl)-3,6-dihydropyridine-1(2H)-carboxylate as an oil that became a white solid on high vacuum.

LRMS (ESI) calc'd for C14H19ClN3O2 [M+H]⁺, 296. found 296.

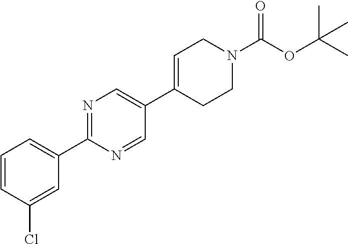

### Step 2. tert-Butyl 4-[2-(3-chlorophenyl)pyrimidin-5-yl]-3,6-dihydropyridine-1(2H)-carboxylate

tert-Butyl 4-[2-(3-chlorophenyl)pyrimidin-5-yl]-3,6-dihydropyridine-1(2H)-carboxylate was prepared from tert-butyl 4-(2-chloropyrimidin-5-yl)-3,6-dihydropyridine-1(2H)-carboxylate according to the procedure described for 2-(3-chlorophenyl)-5-ethoxypyrimidine (Intermediate #86, Step 4).

LRMS (ESI) calc'd for C20H23ClN3O2 [M+H]⁺, 372. found 372.

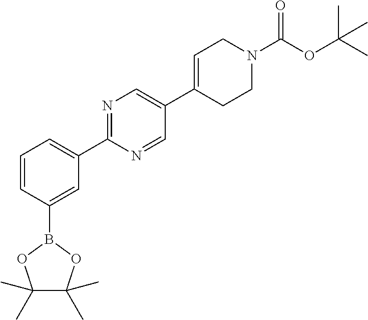

### Step 3. tert-Butyl 4-{2-[3-(4,4,5,5-tetramethyl-1,3,2-dioxaborolan-2-yl)phenyl]pyrimidin-5-yl}-3,6-dihydropyridine-1(2H)-carboxylate

tert-Butyl 4-{2-[3-(4,4,5,5-tetramethyl-1,3,2-dioxaborolan-2-yl)phenyl]pyrimidin-5-yl}-3,6-dihydropyridine-1(2H)-carboxylate was prepared from tert-butyl 4-[2-(3-chlorophenyl)pyrimidin-5-yl]-3,6-dihydropyridine-1(2H)-carboxylate according to the procedure described for 5-ethoxy-2-[3-(4,4,5,5-tetramethyl-1,3,2-dioxaborolan-2-yl)phenyl]pyrimidine (Intermediate #86 Step 5).

LRMS (ESI) calc'd for C26H35BN3O4 [M+H]⁺, 464. found 464.

### Boronic Ester Synthesis Method T

### Intermediate #105

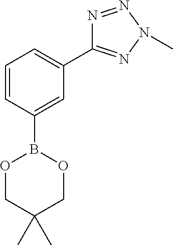

### 5-[3-(5,5-Dimethyl-1,3,2-dioxaborinan-2-yl)phenyl]-2-methyl-2H-tetrazole

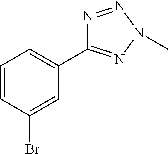

### Step 1. 5-(3-Bromophenyl)-2-methyl-2H-tetrazole

5-(3-Bromophenyl)-2-methyl-2H-tetrazole was prepared from 3-bromobenzonitrile according to the procedures described in WO9527692.

### Step 2. 5-(3-(5,5-Dimethyl-1,3,2-dioxaborinan-2-yl)phenyl-2-methyl-2H-tetrazole

5-[3-(5,5-Dimethyl-1,3,2-dioxaborinan-2-yl)phenyl]-2-methyl-2H-tetrazole was prepared from 5-(3-bromophenyl)-2-methyl-2H-tetrazole according to the procedure described in WO03006464.

### Scheme 4

### Example #1

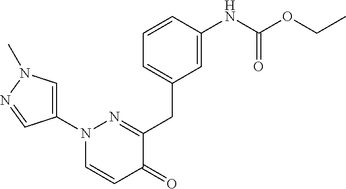

### Ethyl(3-{[1-(1-methyl-1H-pyrazol-4-yl)-4-oxo-1,4-dihydropyridazin-3-yl]methyl}phenyl)carbamate

### Step 1. Ethyl(3-{[1-(1-methyl-1H-pyrazol-4-yl)-4-oxo-1,4-dihydropyridazin-3-yl]methyl}phenyl)carbamate

Ethyl [3-(4,4,5,5-tetramethyl-1,3,2-dioxaborolan-2-yl)phenyl]carbamate (Intermediate #40, 8.88 g, 30.5 mmol); 3-(chloromethyl)-1-(1-methyl-1H-pyrazol-4-yl)pyridazin-4(1H)-one (Intermediate #1, 6.85 g, 30.5 mmol), Na₂CO₃ (6.46 g, 61.0 mmol) and PdCl₂(dppf).DCM (1.25 g, 1.53 mmol) were added to a 3-necked flask and evacuated of air and filled with N₂ (3×). 1,4-Dioxane (122 mL) and water (68 mL) were added and the reaction mixture was degassed by purging with N₂. The reaction mixture was heated to 90° C. for 1 hr. The product was filtered off. The mother liquor was extracted with EtOAc, washed with water and brine, dried, filtered and the solvent was removed in vacuo. The filter cake was washed with EtOAc to yield additional product. The products were combined, EtOAc was added and the organic phase was washed with water. The solvent was evaporated. The product was dissolved in hot MeCN containing 5% water. 20 wt % Darco was added and the suspension stirred for 45 min. The solution was filtered through Celite and the solvent was removed in vacuo to provide ethyl (3-{[1-(1-methyl-1H-pyrazol-4-yl)-4-oxo-1,4-dihydropyridazin-3-yl]methyl}phenyl)carbamate.

LRMS (ESI) calc'd for (C18H19N5O3) [M+H]⁺: 354. Found: 354.

### Scheme 4

### Example #2

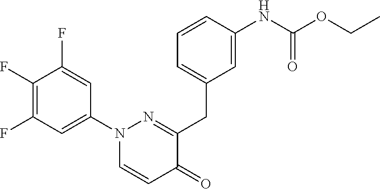

### Ethyl(3-{[4-oxo-1-(3,4,5-trifluorophenyl)-1,4-dihydropyridazin-3-yl]methyl}phenyl)carbamate

### Step 1. Ethyl(3-{[4-oxo-1-(3,4,5-trifluorophenyl)-1,4-dihydropyridazin-3-yl]methyl}phenyl)carbamate

Na₂CO₃ (116 mg, 1.09 mmol) was dissolved in water (0.5 mL) and was added to a solution of 3-(chloromethyl)-1-(3,4,5-trifluorophenyl)pyridazin-4(1H)-one (Intermediate #2, 100 mg, 0.364 mmol) and ethyl [3-(4,4,5,5-tetramethyl-1,3,2-dioxaborolan-2-yl)phenyl]carbamate (Intermediate #40, 159 mg, 0.546 mmol) in DME (1.0 mL) in a pressure tube. Nitrogen was bubbled through the solution for 2 min. Pd(PPh₃)₄ (21 mg, 0.018 mmol) was added, the tube was sealed, and heated to 90° C. Upon completion, the reaction mixture was cooled and diluted with EtOAc. The solution was sequentially washed with saturated NaHCO₃, brine, dried over Na₂SO₄, filtered and concentrated in vacuo. The crude product was purified by reverse phase preparative HPLC (20-100% MeCN—H₂O, 0.05% TFA). Pure fractions were dried down and neutralized with sat. NaHCO₃ solution. The product was extracted with EtOAc, washed with brine, dried with Na₂SO₄, and concentrated in vacuo to afford ethyl(3-{[4-oxo-1-(3,4,5-trifluorophenyl)-1,4-dihydropyridazin-3-yl]methyl}phenyl)carbamate as a solid.

LRMS (ESI) calc'd for C20H17F3N3O3 [M+H]⁺: 405. Found: 405.

### Scheme 4

### Example #3

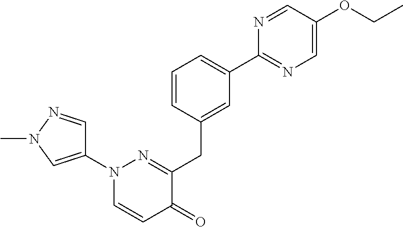

### 3-[3-(5-Ethoxypyrimidin-2-yl)benzyl]-1-(1-methyl-1H pyrazol-4-yl)pyridazin-4(1H)-one

### Step 1. 3-[3-(5-Ethoxypyrimidin-2-yl)benzyl]-1-(1-methyl-1H-pyrazol-4-yl)pyridazin-4(1H)-one

Allylpalldium chloride dimer (0.42 g, 1.16 mmol), DavePhos (1.82 g, 4.63 mmol), water (103 mL) and 2-methyl-THF (516 mL) were added to a flask. The mixture was sparged with N₂ for 20 minutes then 3-(chloromethyl)-1-(1-methyl-1H-pyrazol-4-yl)pyridazin-4(1H)-one (Intermediate #1, 26g, 116 mmol), 5-ethoxy-2-[3-(4,4,5,5-tetramethyl-1,3,2-dioxaborolan-2-yl)phenyl]pyrimidine (Intermediate #86, 37.8 g, 116 mmol) and K₃PO₄ (49.1 g, 231 mmol) were added. The resulting mixture was heated to reflux for 18 hours. The reaction mixture was then cooled to ˜40° C. and diluted with EtOAc and 2-methyl-THF. The suspension was filtered through Celite; eluting with EtOAc. The layers were separated and the aqueous portion extracted with EtOAc. The combined organic extracts were washed with brine. The Celite filter cake was rinsed with DCM until HPLC sampling indicated that all product had been retrieved. The combined DCM portions were washed with brine and combined with the previous organic layer. The volume of the combined solution was reduced to ˜3 L of solvent before drying over MgSO₄ and filtering. To this solution was added 50 wt % KB-G Darco charcoal and the suspension stirred for 3 hours. The charcoal was removed by filtering through Celite and the solvent switched to ˜1.5 L of DCM. The solvent was removed in vacuo while loading onto 175g of silica gel and the residue was purified by flash chromatography (0-7% MeOH-DCM). Pure fractions were collected and reduced to ˜300 mL DCM. EtOAc (1 L) was added and the volume reduced to ˜300 mL and the process repeated twice more. The resulting precipitate was collected by filtration and washed with hexanes. The product-solid was taken up in DCM before concentrating in vacuo while loading onto 150g silica gel and the residue was purified by flash chromatography (0-7% MeOH-DCM). The resulting product was dissolved in 1:1 DCM:THF (1200 mL), 100 wt % Darco G-60 was added and the suspension was stirred for 3 hours. The charcoal was removed by filtering through Celite and the volume-reduced to ˜1 L of solvent. The mixture was solvent switched into EtOAc (1 L×3). The volume was reduced to ˜200 mL before adding hexanes (600 mL) dropwise to the slurry. The product solid was collected by filtration and dried to give 3-[3-(5-ethoxypyrimidin-2-yl)benzyl]-1-(1-methyl-1H-pyrazol-4-yl)pyridazin-4(1H)-one as a white-solid.

LRMS (ESI) calc'd for C21H21N6O2 [M+H]⁺: 389. Found: 389.

### Scheme 4

### Example #4

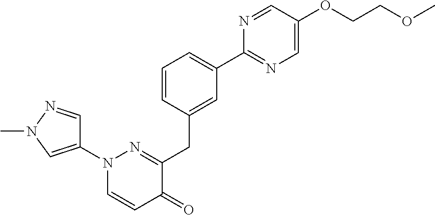

### 3-{3-[5-(2-Methoxyethoxy)pyrimidin-2-yl]benzyl}-1-(1-methyl-1H-pyrazol-4-yl)pyridazin-4(1H1)-one

### Step 1. 3-{3-[5-(2-Methoxyethoxy)pyrimidin-2-yl]benzyl}-1-(1-methyl-1H-pyrazol-4-yl)pyridazin-4(1H)-one

3-(Chloromethyl)-1-(1-methyl-1H-pyrazol-4-yl)pyridazin-4(1H)-one (Intermediate #1, 11.1 g, 49.5 mmol), 5-(2-methoxyethoxy)-2-[3-(4,4,5,5-tetramethyl-1,3,2-dioxaborolan-2-yl)phenyl]pyrimidine (Intermediate #87, 21.1 g, 59.3 mmol), PdCl₂(dppf).DCM (0.81 g, 0.99 mmol) and K₃PO₄ (31.5 g, 148 mmol) were combined in a 1-liter, 3-neck round-bottom flask equipped with a mechanical stirrer, reflux condenser, and nitrogen inlet. To this solid mixture was added DME (225 mL) followed by water (22.5 mL). The resulting reaction mixture was purged with argon (subsurface bubbling) for 15 min, then was heated to 100° C. and stirred for approximately 30 minutes. The reaction was cooled, and diluted with EtOAc (500 mL) and 5% aqueous NH₄Cl (500 mL). After stirring the biphasic mixture for approximately 5 min, the mixture was filtered through Celite and rinsed with EtOAc (2×100 mL). The layers were separated, and the aqueous layer was extracted with EtOAc (3×200 mL). The combined organic layers were dried over sodium sulfate, filtered and concentrated to a crude solid. The solid was dissolved in DCM, and was concentrated to a final volume of approximately 65 mL. Hexanes (65 mL) was added slowly, and an oily precipitate formed. Additional DCM (10 mL) was added and a stirrable solid began to form. Additional hexanes (65 mL) was added to achieve a final solvent ratio of approximately 2:1 hexanes:DCM. The solids were filtered, rinsing the filter cake with the same solvent mixture (2×50 mL) to afford 3-{3-[5=(2-methoxyethoxy)pyrimidin-2-yl]benzyl}-1-(1-methyl-1H-pyrazol-4-yl)pyridazin-4(1H)-one as an off-white solid.

LRMS (ESI) calc'd for C22H23N6O3 [M+H]⁺: 419. Found: 419.

### Scheme 4

### Example #5

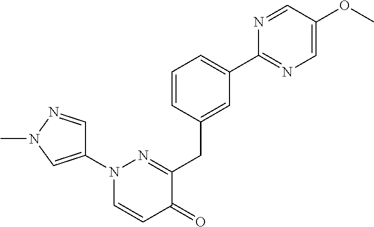

### 3-[3-(5-Methoxypyrimidin-2-yl)benzyl]-1-(1-methyl-1H-pyrazol-4-yl)pyridazin-4(1H)-one

### Step 1. 3-[3-(5-Methoxypyrimidin-2-yl)benzyl]-1-(1-methyl-1H-pyrazol-4-yl)pyridazin-4(1H)-one

3-(Chloromethyl)-1-(1-methyl-1H-pyrazol-4-yl)pyridazin-4(1H)-one (Intermediate #1, 0.655 g, 2.92 mmol), 5-methoxy-2-[3-(4,4,5,5-tetramethyl-1,3,2-dioxaborolan-2-yl)phenyl]pyrimidine (Intermediate #47, 1.00 g, 3.21 mmol), K₃PO₄ (1.86 g, 8.75 mmol) and PdCl₂(dppf).DCM adduct (0.05 g, 0.058 mmol) were taken up in degassed 10:1 DME/water (28 mL) in a 100 mL round bottom-flask. The flask was evacuated and back-filled with N₂ (X3) and the reaction mixture stirred at 100° C. for 70 minutes. Room temperature was attained, saturated NH₄Cl was added and the products extracted into EtOAc (X2). The combined organic extracts were washed with brine, dried over MgSO₄, filtered through Celite and concentrated in vacuo while loading onto silica. Purification of the residue by flash chromatography (MPLC, 0-15% MeOH-EtOAc) gave 3-[3-(5-methoxypyrimidin-2-yl)benzyl]-1-(1-methyl-1H-pyrazol-4-yl)pyridazin-4(1H)-one as a beige solid.

LRMS (ESI) calc'd for C20H19N6O2 [M+H]⁺: 375. Found: 375.

### Scheme 4

### Example #6

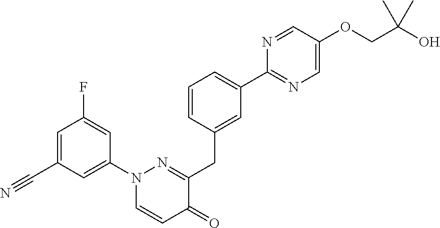

### 3-Fluoro-5-[3-{3-[5-(2-hydroxy-2-methylpropoxy)pyrimidin-2-yl]benzyl}-4-oxopyridazin-1(4H)-yl]-benzonitrile

### Step 1. 3-Fluoro-5-[3-{3-[5-(2-hydroxy-2-methylpropoxy)pyrimidin-2-yl]benzyl}-4-oxopyridazin-1(4H)-yl]benzonitrile

2-Methyl-1-({2-[3-(4,4,5,5-tetramethyl-1,3,2-dioxaborolan-2-yl)phenyl]pyrimidin-5-yl}oxy)propan-2-ol (Intermediate #100, 115 mg, 0.31 mmol), 3-[3-(chloromethyl)-4-oxopyridazin-1(4H)-yl]-5-fluorobenzonitrile (Intermediate #24, 68 mg, 0.258 mmol), K₃PO₄ (164 mg, 0.774 mmol) and PdCl₂(dppf).DCM adduct (4.2 mg, 5.2 μmol) were taken up in de-gassed 10:1 DME:H₂O (2.5 mL) in a 20 mL microwave vial. The vial was evacuated and back-filled with N₂ (X3) and the reaction mixture stirred at 100° C. for 60 minutes. Room temperature was attained and the reaction mixture filtered through Celite, eluting with MeOH. The solvent was removed in vacuo and the residue purified by flash chromatography (MPLC, 0-10% MeOH-EtOAc) followed by flash chromatography (MPLC, 0-5% MeOH-EtOAc) to give product containing an unknown impurity. This product was dissolved in 2 mL of DMSO and purified by mass-triggered reverse phase preparative HPLC. The combined product fractions were neutralized with saturated NaHCO₃ and the product extracted into EtOAc. The organic extract was dried over-MgSO₄ and concentrated in vacuo to give 3-fluoro-5-[3-{3-[5-(2-hydroxy-2-methylpropoxy)pyrimidin-2-yl]benzyl}-4-oxopyridazin-1 (4H)-yl]benzonitrile as a pale yellow solid.

LRMS (ESI) calc'd for C26H23FN5O3 [M+H]⁺: 472. Found: 472.

The following examples were prepared according to Scheme 4 following similar procedures described for Examples #1-6, using intermediates #1-27, 31-38 and #40-61, 63-82, 84, 86-103, 105. (and commercial aryl boronates), which can be achieved by those of ordinary skill in the art of organic synthesis.

| | | | Exact |
|---|---|---|---|
| | | IUPAC | Mass |
| Example | Structure | Name | [M + H]⁺ |
| 7 | 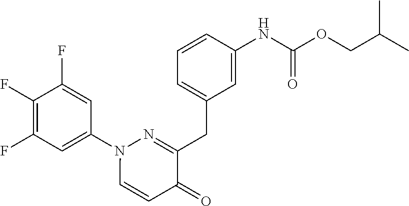 | 2-methylpropyl (3-{[4-oxo-1- (3,4,5-trifluorophenyl)-1,4- dihydropyridazin-3- yl]methyl}phenyl)carbamate | Calc'd 432, found 432 |
| 8 | 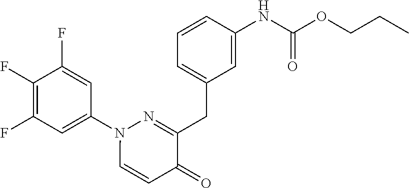 | propyl (3-{[4-oxo-1-(3,4,5- trifluorophenyl)-1,4- dihydropyridazin-3- yl]methyl}phenyl)carbamate | Calc'd 418, found 418 |
| 9 | 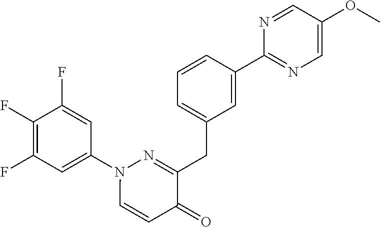 | 3-[3-(5-methoxypyrimidin-2- yl)benzyl]-1-(3,4,5- trifluorophenyl)pyridazin-4(1H)- one | Calc'd 425, found 425 |
| 10 | 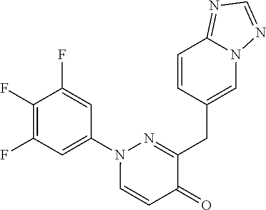 | 3-([1,2,4]triazolo[1,5-a]pyridin- 6-ylmethyl)-1-(3,4,5- trifluorophenyl)pyridazin-4(1H)- one | Calc'd 358, found 358 |
| 11 | 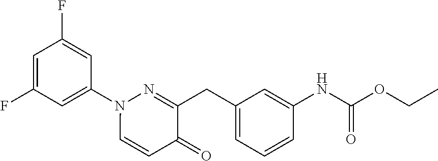 | ethyl (3-{[1-(3,5-difluorophenyl)- 4-oxo-1,4-dihydropyridazin-3- yl]methyl}phenyl)carbamate | Calc'd 386, found 386 |
| 12 | 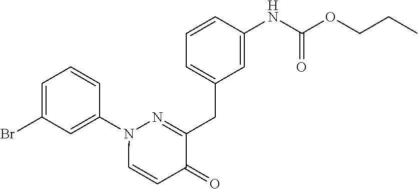 | propyl (3-{[1-(3-bromophenyl)- 4-oxo-1,4-dihydropyridazin-3- yl]methyl}phenyl)carbamate | Calc'd 442, found 442 |
| 13 | 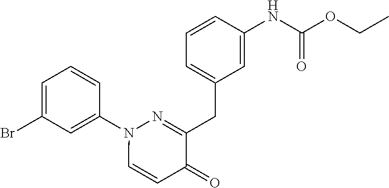 | ethyl (3-{[1-(3-bromophenyl)-4- oxo-1,4-dihydropyridazin-3- yl]methyl}phenyl)carbamate | Calc'd 428, found 428 |
| 14 | 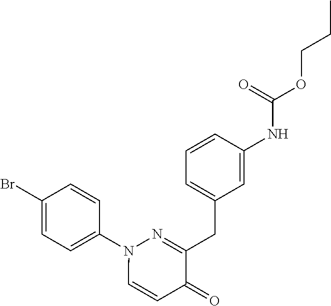 | propyl (3-{[1-(4-bromophenyl)- 4-oxo-1,4-dihydropyridazin-3- yl]methyl}phenyl)carbamate | Calc'd 442, found 442 |
| 15 | 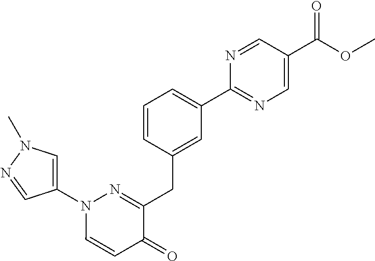 | methyl 2-(3-{[1-(1-methyl-1H- pyrazol-4-yl)-4-oxo-1,4- dihydropyridazin-3- yl]methyl}phenyl)pyrimidine-5- carboxylate | Calc'd 403, found 403 |
| 16 | 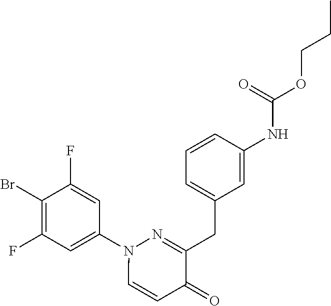 | propyl (3-{[1-(4-bromo-3,5- difluorophenyl)-4-oxo-1,4- dihydropyridazin-3- yl]methyl}phenyl)carbamate | Calc'd 478, found 478 |
| 17 | 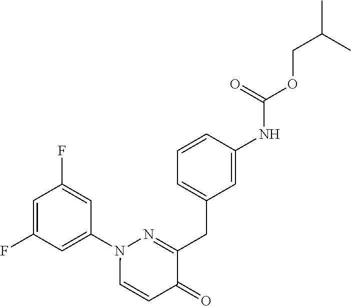 | 2-methylpropyl (3-{[1-(3,5- difluorophenyl)-4-oxo-1,4- dihydropyridazin-3- yl]methyl}phenyl)carbamate | Calc'd 414, found 414 |
| 18 | 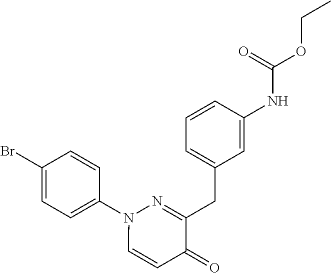 | ethyl (3-{[1-(4-bromophenyl)-4- oxo-1,4-dihydropyridazin-3- yl]methyl}phenyl)carbamate | Calc'd 428, found 428 |
| 19 | 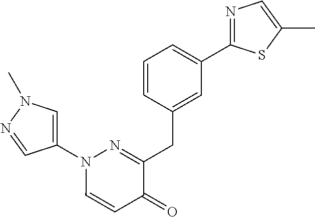 | 1-(1-methyl-1H-pyrazol-4-yl)-3- [3-(5-methyl-1,3-thiazol-2- yl)benzyl]pyridazin-4(1H)-one | Calc'd 364, found 364 |
| 20 | 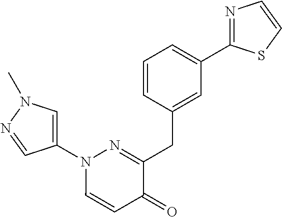 | 1-(1-methyl-1H-pyrazol-4-yl)-3- [3-(1,3-thiazol-2- yl)benzyl]pyridazin-4(1H)-one | Calc'd 350, found 350 |
| 21 | 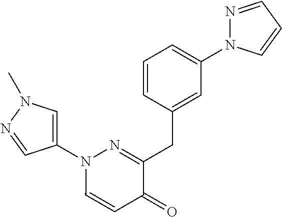 | 1-(1-methyl-1H-pyrazol-4-yl)-3- [3-(1H-pyrazol-1- yl)benzyl]pyridazin-4(1H)-one | Calc'd 333, found 333 |
| 22 | 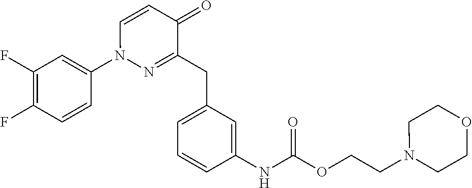 | 2-morpholin-4-ylethyl (3-{[1- (3,4-difluorophenyl)-4-oxo-1,4- dihydropyridazin-3- yl]methyl}phenyl)carbamate | Calc'd 471, found 471 |
| 23 | 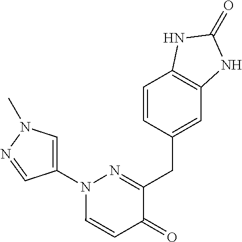 | 5-{[1-(1-methyl-1H-pyrazol-4- yl)-4-oxo-1,4-dihydropyridazin- 3-yl]methyl}-1,3-dihydro-2H- benzimidazol-2-one | Calc'd 323, found 323 |
| 24 | 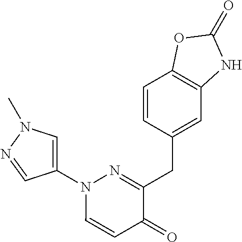 | 5-{[1-(1-methyl-1H-pyrazol-4- yl)-4-oxo-1,4-dihydropyridazin- 3-yl]methyl}-1,3-benzoxazol- 2(3H)-one | Calc'd 324, found 324 |
| 25 | 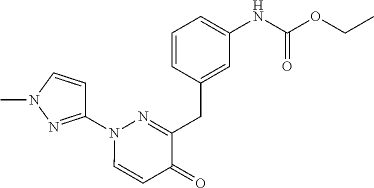 | ethyl (3-{[1-(1-methyl-1H- pyrazol-3-yl)-4-oxo-1,4- dihydropyridazin-3- yl]methyl}phenyl)carbamate | Calc'd 354, found 354 |
| 26 | 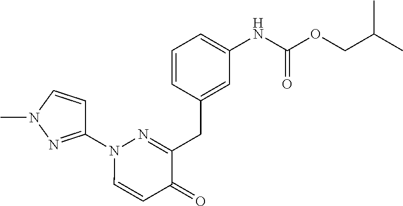 | 2-methylpropyl (3-{[1-(1-methyl- 1H-pyrazol-3-yl)-4-oxo-1,4- dihydropyridazin-3- yl]methyl}phenyl)carbamate | Calc'd 382, found 382 |
| 27 | 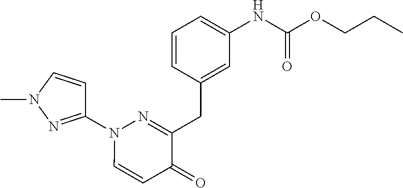 | propyl (3-{[1-(1-methyl-1H- pyrazol-3-yl)-4-oxo-1,4- dihydropyridazin-3- yl]methyl}phenyl)carbamate | Calc'd 368, found 368 |
| 28 | 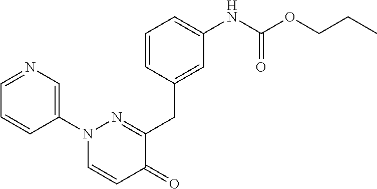 | propyl {3-[(4-oxo-1-pyridin-3-yl- 1,4-dihydropyridazin-3- yl)methyl]phenyl}carbamate | Calc'd 365, found 365 |
| 29 | 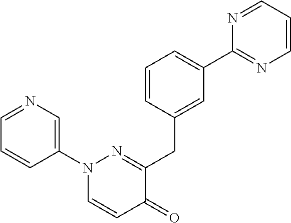 | 1-pyridin-3-yl-3-(3-pyrimidin-2- ylbenzyl)pyridazin-4(1H)-one | Calc'd 342, found 342 |
| 30 | 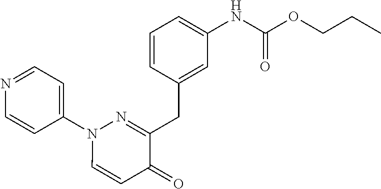 | propyl {3-[(4-oxo-1-pyridin-4-yl- 1,4-dihydropyridazin-3- yl)methyl]phenyl}carbamate | Calc'd 365, found 365 |
| 31 | 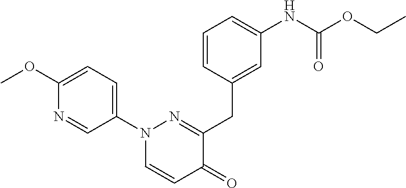 | ethyl (3-{[1-(6-methoxypyridin- 3-yl)-4-oxo-1,4- dihydropyridazin-3- yl]methyl}phenyl)carbamate | Calc'd 381, found 381 |
| 32 | 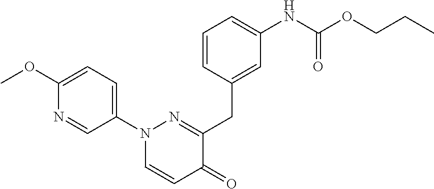 | propyl (3-{[1-(6-methoxypyridin- 3-yl)-4-oxo-1,4- dihydropyridazin-3- yl]methyl}phenyl)carbamate | Calc'd 395, found 395 |
| 33 | 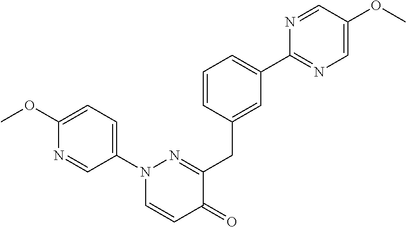 | 1-(6-methoxypyridin-3-yl)-3-[3- (5-methoxypyrimidin-2- yl)benzyl]pyridazin-4(1H)-one | Calc'd 402, found 402 |
| 34 | 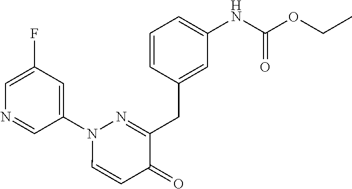 | ethyl (3-{[1-(5-fluoropyridin-3- yl)-4-oxo-1,4-dihydropyridazin- 3-yl]methyl}phenyl)carbamate | Calc'd 369, found 369 |
| 35 | 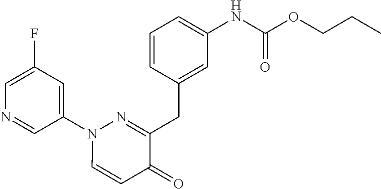 | propyl (3-{[1-(5-fluoropyridin-3- yl)-4-oxo-1,4-dihydropyridazin- 3-yl]methyl}phenyl)carbamate | Calc'd 383, found 383 |
| 36 | 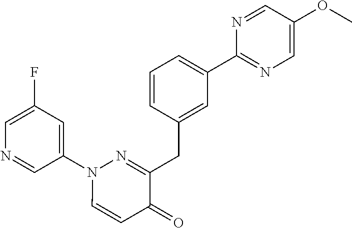 | 1-(5-fluoropyridin-3-yl)-3-[3-(5- methoxypyrimidin-2- yl)benzyl]pyridazin-4(1H)-one | Calc'd 390, found 390 |
| 37 | 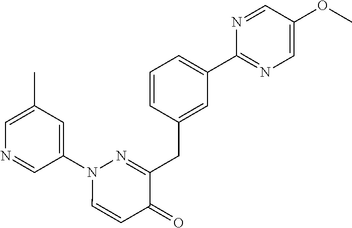 | 3-[3-(5-methoxypyrimidin-2- yl)benzyl]-1-(5-methylpyridin-3- yl)pyridazin-4(1H)-one | Calc'd 386, found 386 |
| 38 | 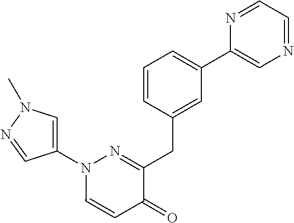 | 1-(1-methyl-1H-pyrazol-4-yl)-3- [3-(pyrazin-2- yl)benzyl]pyridazin-4(1H)-one | Calc'd 345, found |
| 39 | 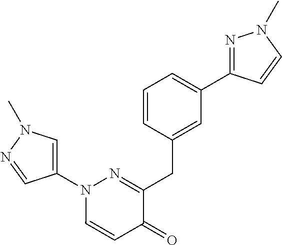 | 1-(1-methyl-1H-pyrazol-4-yl)-3- [3-(1-methyl-1H-pyrazol-3- yl)benzyl]pyridazin-4(1H)-one | Calc'd 347, found 347 |
| 40 | 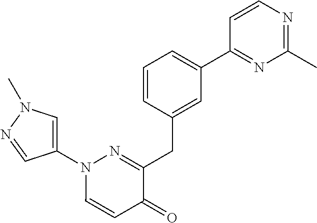 | 1-(1-methyl-1H-pyrazol-4-yl)-3- [3-(2-methylpyrimidin-4- yl)benzyl]pyridazin-4(1H)-one | Calc'd 359, found 359 |
| 41 | 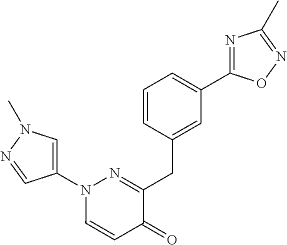 | 3-[3-(3-methyl-1,2,4-oxadiazol- 5-yl)benzyl]-1-(1-methyl-1H- pyrazol-4-yl)pyridazin-4(1H)-one | Calc'd 349, found 349 |
| 42 | 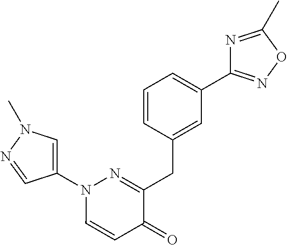 | 3-[3-(5-methyl-1,2,4-oxadiazol- 3-yl)benzyl]-1-(1-methyl-1H- pyrazol-4-yl)pyridazin-4(1H)-one | Calc'd 349, found 349 |
| 43 | 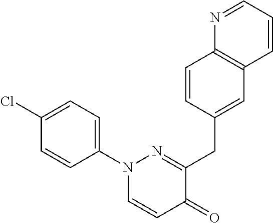 | 1-(4-chlorophenyl)-3-(quinolin-6- ylmethyl)pyridazin-4(1H)-one | Calc'd 348, found 348 |
| 44 | 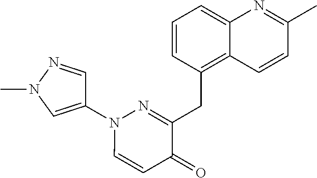 | 1-(1-methyl-1H-pyrazol-4-yl)-3- [(2-methylquinolin-5- yl)methyl]pyridazin-4(1H)-one | Calc'd 332, found 332 |
| 45 | 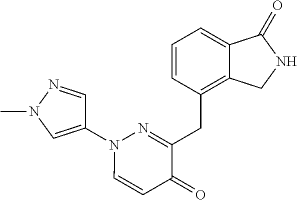 | 4-{[1-(1-methyl-1H-pyrazol-4- yl)-4-oxo-1,4-dihydropyridazin- 3-yl]methyl}-2,3-dihydro-1H- isoindol-1-one | Calc'd 322, found 322 |
| 46 | 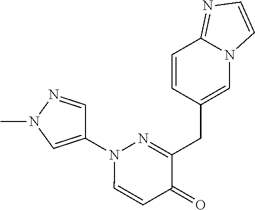 | 3-(imidazo[1,2-a]pyridin-6- ylmethyl)-1-(1-methyl-1H- pyrazol-4-yl)pyridazin-4(1H)-one | Calc'd 307, found 307 |
| 47 | 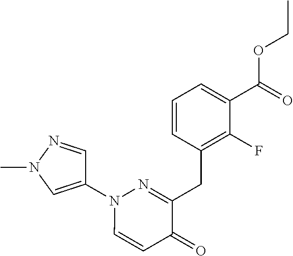 | ethyl 2-fluoro-3-{[1-(1-methyl- 1H-pyrazol-4-yl)-4-oxo-1,4- dihydropyridazin-3- yl]methyl}benzoate | Calc'd 357, found 357 |
| 48 | 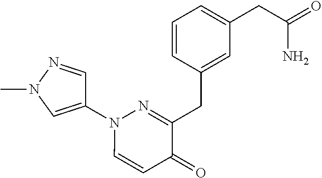 | 2-(3-{[1-(1-methyl-1H-pyrazol- 4-yl)-4-oxo-1,4- dihydropyridazin-3- yl]methyl}phenyl)acetamide | Calc'd 324, found 324 |
| 49 | 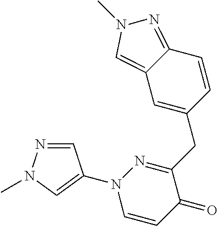 | 3-[(2-methyl-2H-indazol-5- yl)methyl]-1-(1-methyl-1H- pyrazol-4-yl)pyridazin-4(1H)-one | Calc'd 321, found 321 |
| 50 | 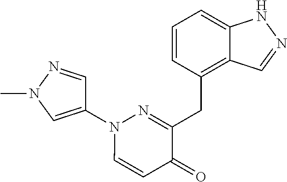 | 3-(1H-indazol-4-ylmethyl)-1-(1- methyl-1H-pyrazol-4- yl)pyridazin-4(1H)-one | Calc'd 307, found 307 |
| 51 | 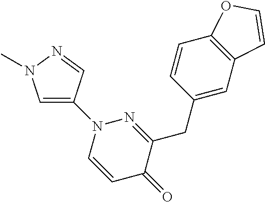 | 3-(1-benzofuran-5-ylmethyl)-1- (1-methyl-1H-pyrazol-4- yl)pyridazin-4(1H)-one | Calc'd 307, found 307 |
| 52 | 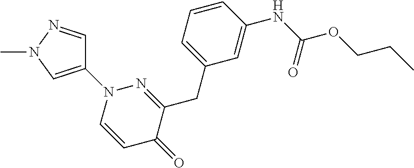 | propyl (3-{[1-(1-methyl-1H- pyrazol-4-yl)-4-oxo-1,4- dihydropyridazin-3- yl]methyl}phenyl)carbamate | Calc'd 368, found 368 |
| 53 | 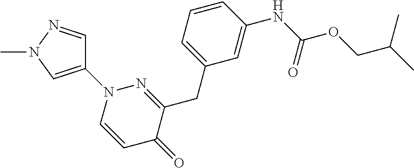 | 2-methylpropyl (3-{[1-(1-methyl- 1H-pyrazol-4-yl)-4-oxo-1,4- dihydropyridazin-3- yl]methyl}phenyl)carbamate | Calc'd 382, found 382 |
| 54 | 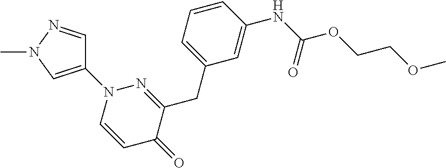 | 2-methoxyethyl (3-{[1-(1- methyl-1H-pyrazol-4-yl)-4-oxo- 1,4-dihydropyridazin-3- yl]methyl}phenyl)carbamate | Calc'd 384, found 384 |
| 55 | 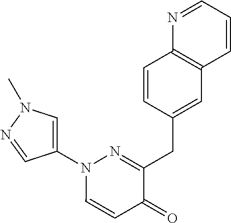 | 1-(1-methyl-1H-pyrazol-4-yl)-3- (quinolin-6-ylmethyl)pyridazin- 4(1H)-one | Calc'd 318, found 318 |
| 56 | 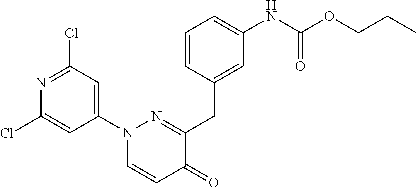 | propyl (3-{[1-(2,6- dichloropyridin-4-yl)-4-oxo-1,4- dihydropyridazin-3- yl]methyl}phenyl)carbamate | Calc'd 433, found 433 |
| 57 | 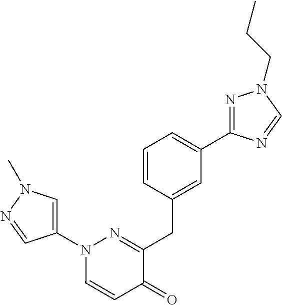 | 1-(1-methyl-1H-pyrazol-4-yl)-3- [3-(1-propyl-1H-1,2,4-traizol-3- yl)benzyl]pyridazin-4(1H)-one | Calc'd 376, found 376 |
| 58 | 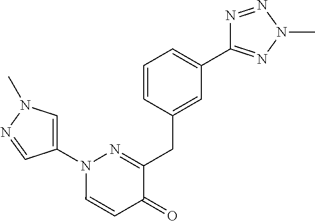 | 1-(1-methyl-1H-pyrazol-4-yl)-3- [3-(2-methyl-2H-tetrazol-5- yl)benzyl]pyridazin-4(1H)-one | Calc'd 349, found 349 |
| 59 | 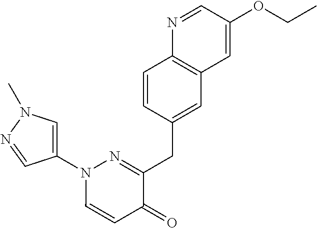 | 3-[(3-ethoxyquinolin-6- yl)methyl]-1-(1-methyl-1H- pyrazol-4-yl)pyridazin-4(1H)-one | Calc'd 362, found 362 |
| 60 | 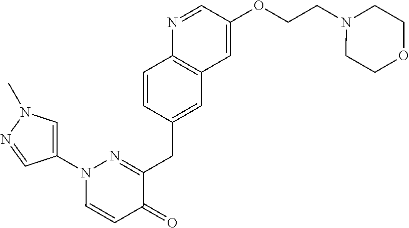 | 1-(1-methyl-1H-pyrazol-4-yl)-3- {[3-(2-morpholin-4- ylethoxy)quinolin-6- yl]methyl}pyridazin-4(1H)-one | Calc'd 447, found 447 |
| 61 | 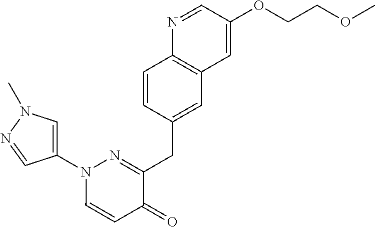 | 3-{[3-(2- methoxyethoxy)quinolin-6- yl]methyl}-1-(1-methyl-1H- pyrazol-4-yl)pyridazin-4(1H)-one | Calc'd 392, found 392 |
| 62 | 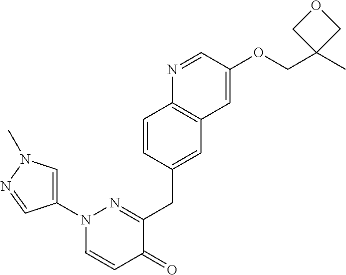 | 3-({3-[(3-methyloxetan-3- yl)methoxy]quinolin-6- yl}methyl)-1-(1-methyl-1H- pyrazol-4-yl)pyridazin-4(1H)-one | Calc'd 418, found 418 |
| 63 | 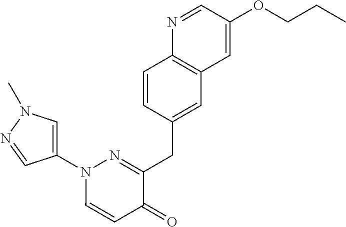 | 1-(1-methyl-1H-pyrazol-4-yl)-3- [(3-propoxyquinolin-6- yl)methyl]pyridazin-4(1H)-one | Calc'd 376, found 376 |
| 64 | 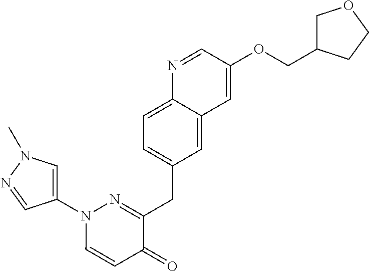 | rac-1-(1-methyl-1H-pyrazol-4- yl)-3-{[3-(tetrahydrofuran-3- ylmethoxy)quinolin-6- yl]methyl}pyridazin-4(1H)-one | Calc'd 418, found 418 |
| 65 | 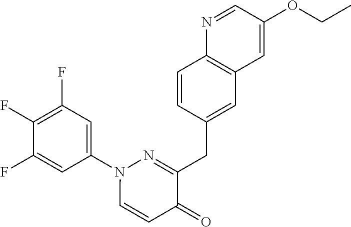 | 3-[(3-ethoxyquinolin-6- yl)methyl]-1-(3,4,5- trifluorophenyl)pyridazin-4(1H)- one | Calc'd 412, found 412 |
| 66 | 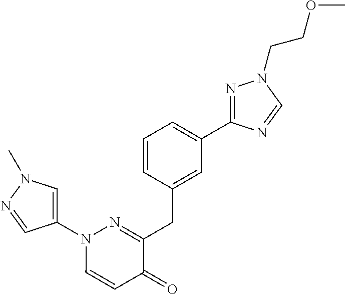 | 3-{3-[1-(2-methoxyethyl)-1H- 1,2,4-triazol-3-yl]benzyl}-1-(1- methyl-1H-pyrazol-4- yl)pyridazin-4(1H)-one | Calc'd 392, found 392 |
| 67 | 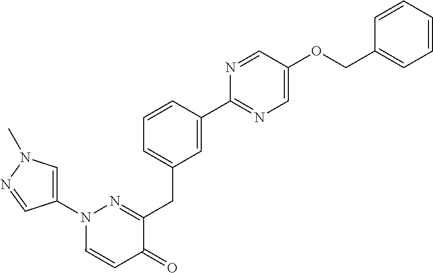 | 3-{3-[5-(benzyloxy)pyrimidin-2- yl]benzyl}-1-(1-methyl-1H- pyrazol-4-yl)pyridazin-4(1H)-one | Calc'd 451, found 451 |
| 68 | 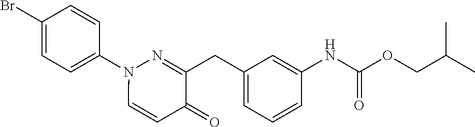 | 2-methylpropyl (3-{[1-(4- bromophenyl)-4-oxo-1,4- dihydropyridazin-3- yl]methyl}phenyl)carbamate | Calc'd 456, found 456 |
| 69 | 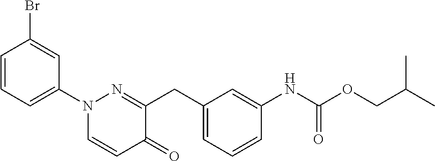 | 2-methylpropyl (3-{[1-(3- bromophenyl)-4-oxo-1,4- dihydropyridazin-3- yl]methyl}phenyl)carbamate | Calc'd 456, found 456 |
| 70 | 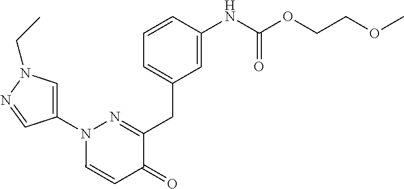 | 2-methoxyethyl (3-{[1-(1-ethyl- 1H-pyrazol-4-yl)-4-oxo-1,4- dihydropyridazin-3- yl]methyl}phenyl)carbamate | Calc'd 398, found 398 |
| 71 | 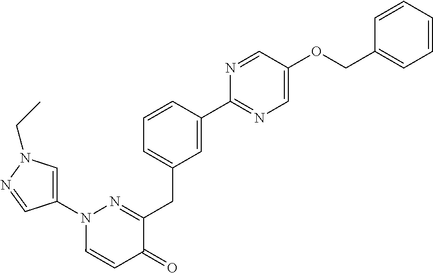 | 3-{3-[5-(benzyloxy)pyrimidin-2- yl]benzyl}-1-(1-ethyl-1H- pyrazol-4-yl)pyridazin-4(1H)-one | Calc'd 465, found 465 |
| 72 | 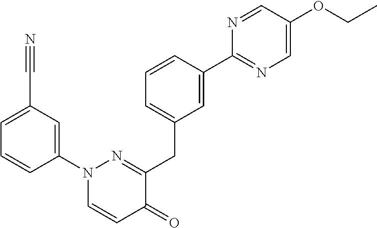 | 3-[3-[3-(5-ethoxypyrimidin-2- yl)benzyl]-4-oxopyridazin-1(4H)- yl]benzonitrile | Calc'd 410, found 410 |
| 73 | 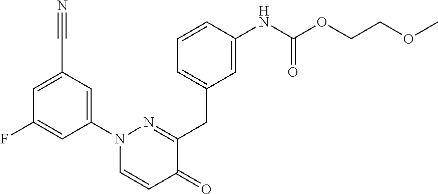 | 2-methoxyethyl (3-{[1-(3-cyano- 5-fluorophenyl)-4-oxo-1,4- dihydropyridazin-3- yl]methyl}phenyl)carbamate | Calc'd 423, found 423 |
| 74 | 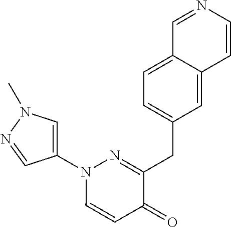 | 3-(isoquinolin-6-ylmethyl)-1-(1- methyl-1H-pyrazol-4- yl)pyridazin-4(1H)-one | Calc'd 318, found 318 |
| 75 | 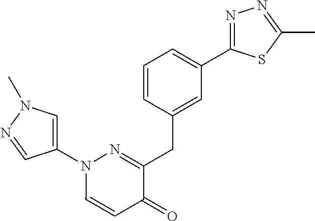 | 1-(1-methyl-1H-pyrazol-4-yl)-3- [3-(5-methyl-1,3,4-thiadiazol-2- yl)benzyl]pyridazin-4(1H)-one | Calc'd 365, found 365 |
| 76 | 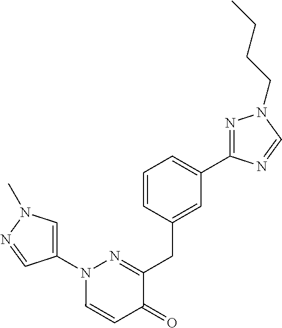 | 3-[3-(1-butyl-1H-1,2,4-triazol-3- yl)benzyl]-1-(1-methyl-1H- pyrazol-4-yl)pyridazin-4(1H)-one | Calc'd 390, found 390 |
| 77 | 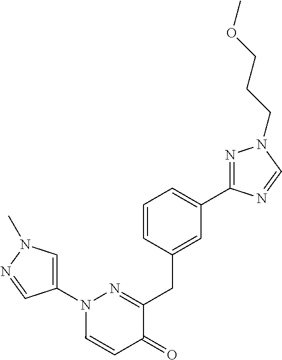 | 3-{3-[1-(3-methoxypropyl)-1H- 1,2,4-triazol-3-yl]benzyl}-1-(1- methyl-1H-pyrazol-4- yl)pyridazin-4(1H)-one | Calc'd 406, found 406 |
| 78 | 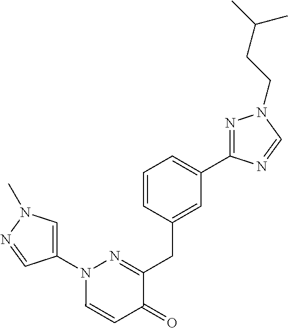 | 3-{3-[1-(3-methylbutyl)-1H- 1,2,4-triazol-3-yl]benzyl}-1-(1- methyl-1H-pyrazol-4- yl)pyridazin-4(1H)-one | Calc'd 404, found 404 |
| 79 | 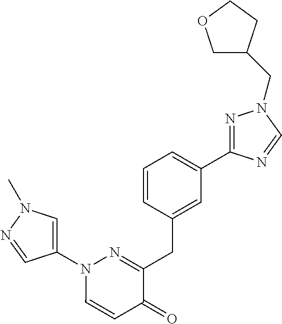 | rac-1-(1-methyl-1H-pyrazol-4- yl)-3-{3-[1-(tetrahydrofuran-3- ylmethyl)-1H-1,2,4-triazol-3- yl]benzyl}pyridazin-4(1H)-one | Calc'd 418, found 418 |
| 80 | 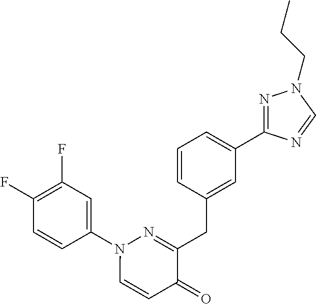 | 1-(3,4-difluorophenyl)-3-[3-(1- propyl-1H-1,2,4-triazol-3- yl)benzyl]pyridazin-4(1H)-one | Calc'd 408, found 408 |
| 81 | 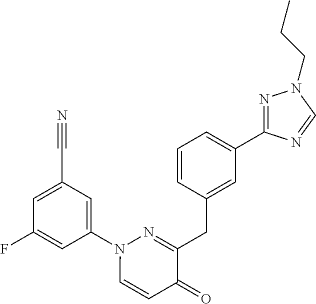 | 3-fluoro-5-{4-oxo-3-[3-(1- propyl-1H-1,2,4-triazol-3- yl)benzyl]pyridazin-1(4H)- yl}benzonitrile | Calc'd 415, found 415 |
| 82 | 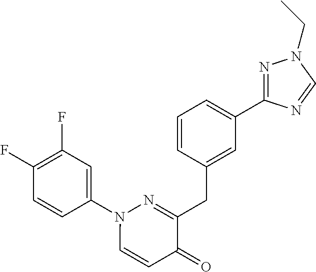 | 1-(3,4-difluorophenyl)-3-[3-(1- ethyl-1H-1,2,4-triazol-3- yl)benzyl]pyridazin-4(1H)-one | Calc'd 394, found 394 |
| 83 | 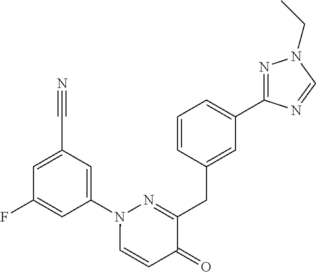 | 3-{3-[3-(1-ethyl-1H-1,2,4-triazol- 3-yl)benzyl]-4-oxopyridazin- 1(4H)-yl}-5-fluorobenzonitrile | Calc'd 401, found 401 |
| 84 | 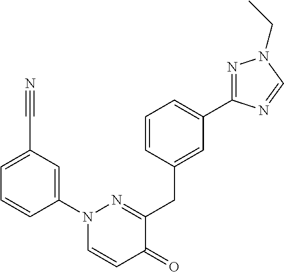 | 3-{3-[3-(1-ethyl-1H-1,2,4-triazol- 3-yl)benzyl]-4-oxopyridazin- 1(4H)-yl}benzonitrile | Calc'd 383, found 383 |
| 85 | 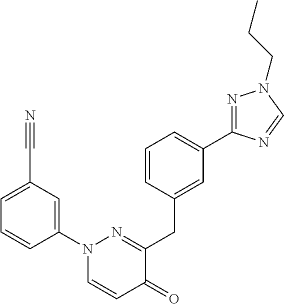 | 3-{4-oxo-3-[3-(1-propyl-1H- 1,2,4-triazol-3- yl)benzyl]pyridazin-1(4H)- yl}benzonitrile | Calc'd 397, found 397 |
| 86 | 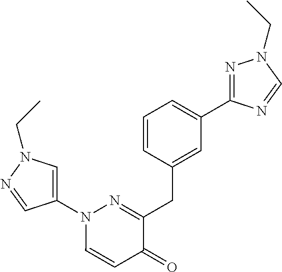 | 1-(1-ethyl-1H-pyrazol-4-yl)-3-[3- (1-ethyl-1H-1,2,4-triazol-3- yl)benzyl]pyridazin-4(1H)-one | Calc'd 376, found 376 |
| 87 | 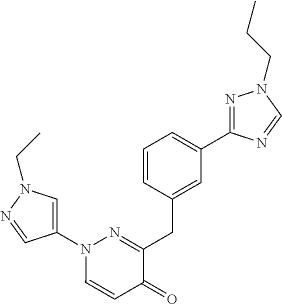 | 1-(1-ethyl-1H-pyrazol-4-yl)-3-[3- (1-propyl-1H-1,2,4-triazol-3- yl)benzyl]pyridazin-4(1H)-one | Calc'd 390, found 390 |
| 88 | 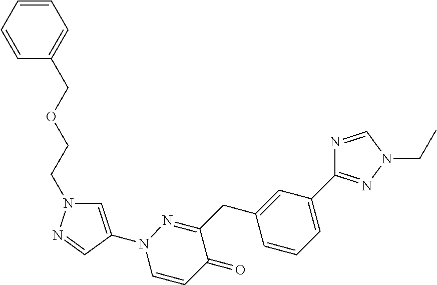 | 1-{1-[2-(benzyloxy)ethyl]-1H- pyrazol-4-yl}-3-[3-(1-ethyl-1H- 1,2,4-triazol-3- yl)benzyl]pyridazin-4(1H)-one | Calc'd 482, found 482 |
| 89 | 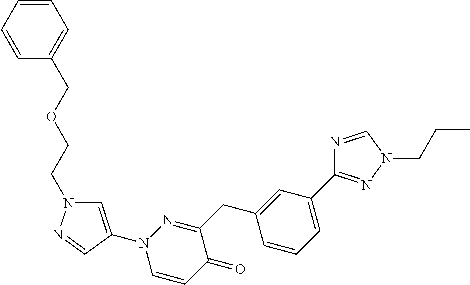 | 1-{1-[2-(benzyloxy)ethyl]-1H- pyrazol-4-yl}-3-[3-(1-propyl-1H- 1,2,4-triazol-3- yl)benzyl]pyridazin-4(1H)-one | Calc'd 496, found 496 |
| 90 | 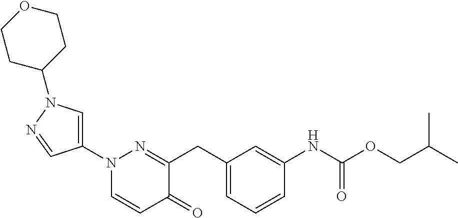 | 2-methylpropyl [3-({4-oxo-1-[1- (tetrahydro-2H-pyran-4-yl)-1H- pyrazol-4-yl]-1,4- dihydropyridazin-3- yl}methyl)phenyl]carbamate | Calc'd 452, found 452 |
| 91 | 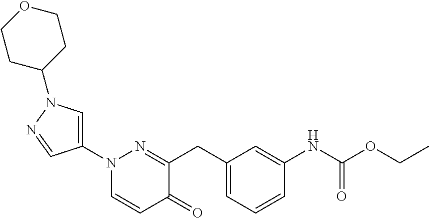 | ethyl [3-({4-oxo-1-[1- (tetrahydro-2H-pyran-4-yl)-1H- pyrazol-4-yl]-1,4- dihydropyridazin-3- yl}methyl)phenyl]carbamate | Calc'd 424, found 424 |
| 92 | 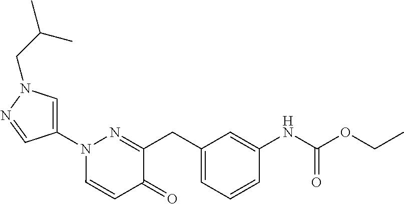 | ethyl [3-({1-[1-(2-methylpropyl)- 1H-pyrazol-4-yl]-4-oxo-1,4- dihydropyridazin-3- yl}methyl)phenyl]carbamate | Calc'd 396, found 396 |
| 93 | 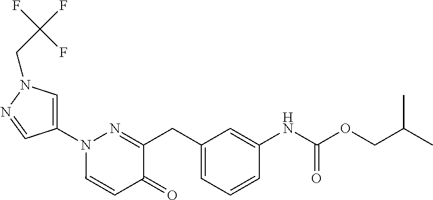 | 2-methylpropyl [3-({4-oxo-1-[1- (2,2,2-trifluoroethyl)-1H-pyrazol- 4-yl]-1,4-dihydropyridazin-3- yl}methyl)phenyl]carbamate | Calc'd 450, found 450 |
| 94 | 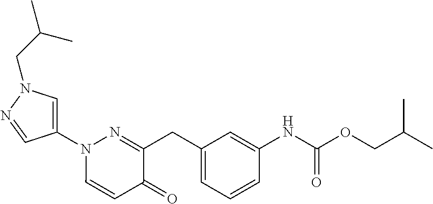 | 2-methylpropyl [3-({1-[1-(2- methylpropyl)-1H-pyrazol-4-yl]- 4-oxo-1,4-dihydropyridazin-3- yl}methyl)phenyl]carbamate | Calc'd 424, found 424 |
| 95 | 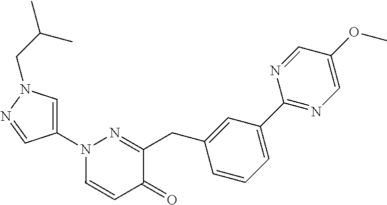 | 3-[3-(5-methoxypyrimidin-2- yl)benzyl]-1-[1-(2- methylpropyl)-1H-pyrazol-4- yl]pyridazin-4(1H)-one | Calc'd 417, found 417 |
| 96 | 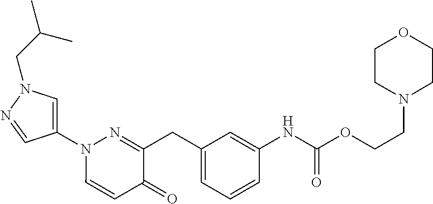 | 2-morpholin-4-ylethyl [3-({1-[1- (2-methylpropyl)-1H-pyrazol-4- yl]-4-oxo-1,4-dihydropyridazin- 3-yl}methyl)phenyl]carbamate | Calc'd 481, found 481 |
| 97 | 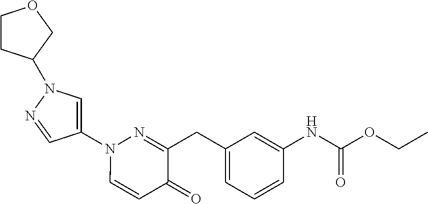 | rac-ethyl [3-({4-oxo-1-[1- (tetrahydrofuran-3-yl)-1H- pyrazol-4-yl]-1,4- dihydropyridazin-3- yl}methyl)phenyl]carbamate | Calc'd 410, found 410 |
| 98 | 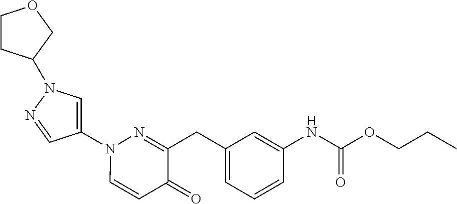 | rac-propyl [3-({4-oxo-1-[1- (tetrahydrofuran-3-yl)-1H- pyrazol-4-yl]-1,4- dihydropyridazin-3- yl}methyl)phenyl]carbamate | Calc'd 424, found 424 |
| 99 | 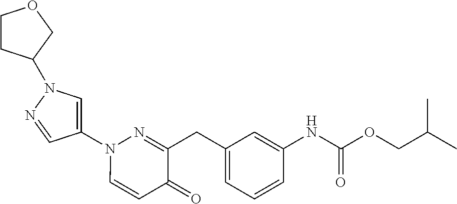 | rac-2-methylpropyl [3-({4-oxo-1- [1-(tetrahydrofuran-3-yl)-1H- pyrazol-4-yl]-1,4- dihydropyridazin-3- yl}methyl)phenyl]carbamate | Calc'd 438, found 438 |
| 100 | 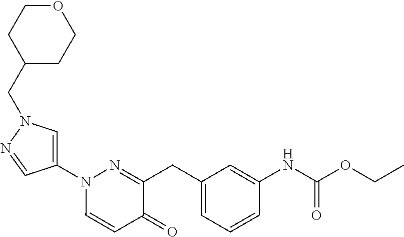 | ethyl [3-({4-oxo-1-[1- (tetrahydro-2H-pyran-4- ylmethyl)-1H-pyrazol-4-yl]-1,4- dihydropyridazin-3- yl}methyl)phenyl]carbamate | Calc'd 438, found 438 |
| 101 | 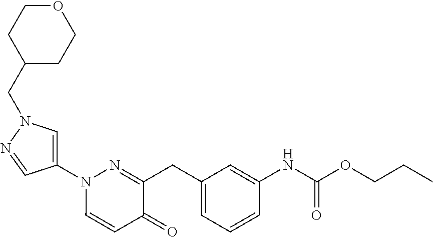 | propyl [3-({4-oxo-1-[1- (tetrahydro-2H-pyran-4- ylmethyl)-1H-pyrazol-4-yl]-1,4- dihydropyridazin-3- yl}methyl)phenyl]carbamate | Calc'd 452, found 452 |
| 102 | 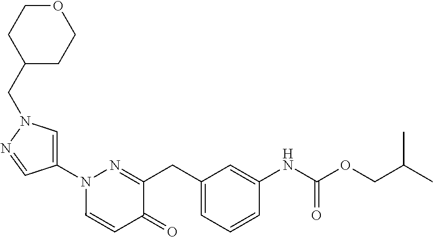 | 2-methylpropyl [3-({4-oxo-1-[1- (tetrahydro-2H-pyran-4- ylmethyl)-1H-pyrazol-4-yl]-1,4- dihydropyridazin-3- yl}methyl)phenyl]carbamate | Calc'd 466, found 466 |
| 103 | 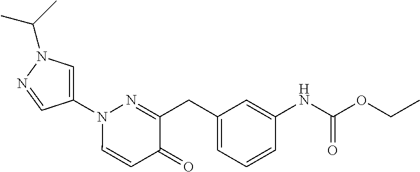 | ethyl [3-({1-[1-(1-methylethyl)- 1H-pyrazol-4-yl]-4-oxo-1,4- dihydropyridazin-3- yl}methyl)phenyl]carbamate | Calc'd 382, found 382 |
| 104 | 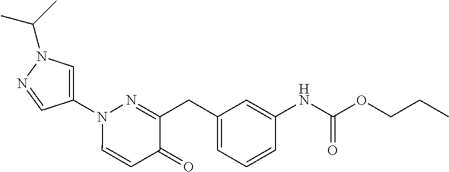 | propyl [3-({1-[1-(1-methylethyl)- 1H-pyrazol-4-yl]-4-oxo-1,4- dihydropyridazin-3- yl}methyl)phenyl]carbamate | Calc'd 396, found 396 |
| 105 | 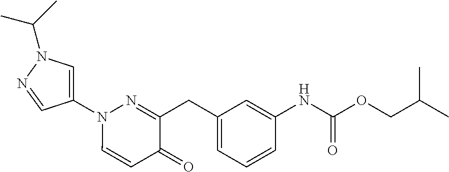 | 2-methylpropyl [3-({1-[1-(1- methylethyl)-1H-pyrazol-4-yl]-4- oxo-1,4-dihydropyridazin-3- yl}methyl)phenyl]carbamate | Calc'd 410, found 410 |
| 106 | 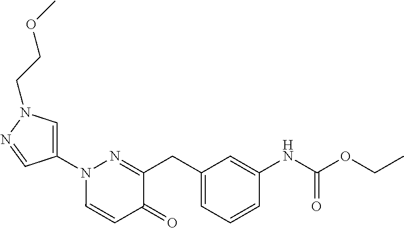 | ethyl [3-({1-[1-(2-methoxyethyl)- 1H-pyrazol-4-yl]-4-oxo-1,4- dihydropyridazin-3- yl}methyl)phenyl]carbamate | Calc'd 398, found 398 |
| 107 | 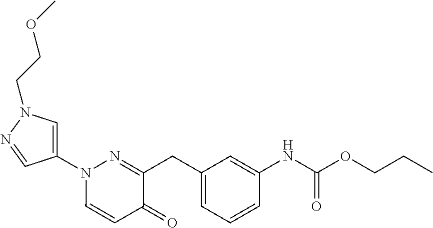 | propyl [3-({1-[1-(2- methoxyethyl)-1H-pyrazol-4-yl]- 4-oxo-1,4-dihydropyridazin-3- yl}methyl)phenyl]carbamate | Calc'd 412, found 412 |
| 108 | 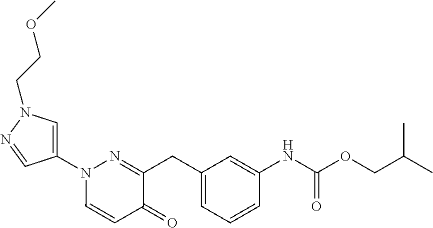 | 2-methylpropyl [3-({1-[1-(2- methoxyethyl)-1H-pyrazol-4-yl]- 4-oxo-1,4-dihydropyridazin-3- yl}methyl)phenyl]carbamate | Calc'd 426, found 426 |
| 109 | 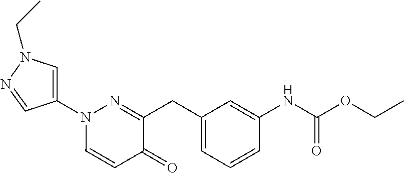 | ethyl (3-{[1-(1-ethyl-1H-pyrazol- 4-yl)-4-oxo-1,4- dihydropyridazin-3- yl]methyl}phenyl)carbamate | Calc'd 368, found 368 |
| 110 | 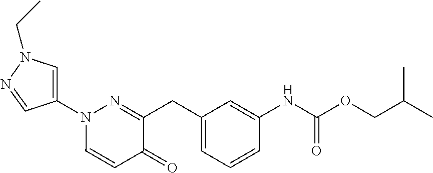 | 2-methylpropyl (3-{[1-(1-ethyl- 1H-pyrazol-4-yl)-4-oxo-1,4- dihydropyridazin-3- yl]methyl}phenyl)carbamate | Calc'd 396, found 396 |
| 111 | 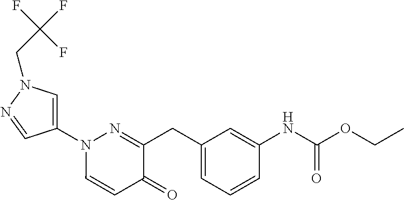 | ethyl [3-({4-oxo-1-[1-(2,2,2- trifluoroethyl)-1H-pyrazol-4-yl]- 1,4-dihydropyridazin-3- yl}methyl)phenyl]carbamate | Calc'd 422, found 422 |
| 112 | 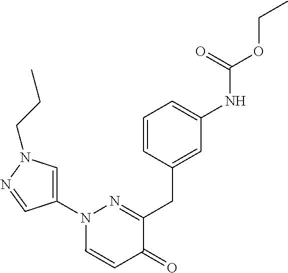 | ethyl (3-{[4-oxo-1-(1-propyl-1H- pyrazol-4-yl)-1,4- dihydropyridazin-3- yl]methyl}phenyl)carbamate | Calc'd 382, found 382 |
| 113 | 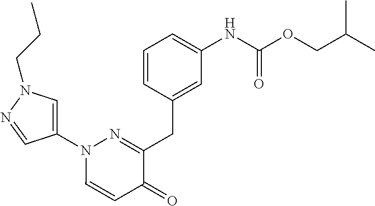 | 2-methylpropyl (3-{[4-oxo-1-(1- propyl-1H-pyrazol-4-yl)-1,4- dihydropyridazin-3- yl]methyl}phenyl)carbamate | Calc'd 410, found 410 |
| 114 | 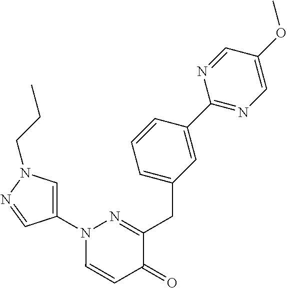 | 3-[3-(5-methoxypyrimidin-2- yl)benzyl]-1-(1-propyl-1H- pyrazol-4-yl)pyridazin-4(1H)-one | Calc'd 403, found 403 |
| 115 | 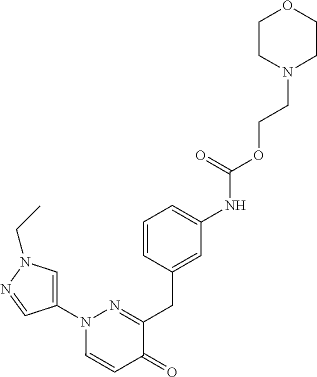 | 2-morpholin-4-ylethyl (3-{[1-(1- ethyl-1H-pyrazol-4-yl)-4-oxo- 1,4-dihydropyridazin-3- yl]methyl}phenyl)carbamate | Calc'd 453, found 453 |
| 116 | 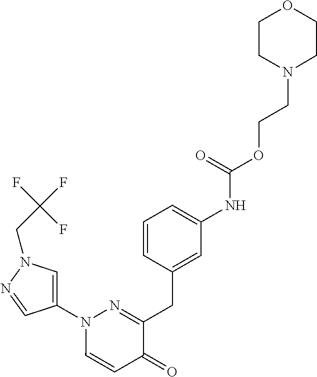 | 2-morpholin-4-ylethyl [3-({4- oxo-1-[1-(2,2,2-trifluoroethyl)- 1H-pyrazol-4-yl]-1,4- dihydropyridazin-3- yl}methyl)phenyl]carbamate | Calc'd 507, found 507 |
| 117 | 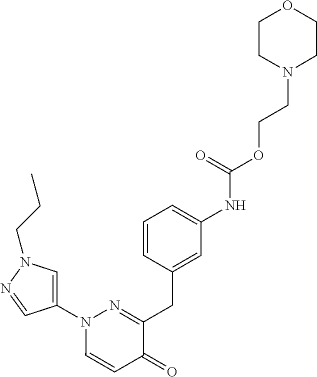 | 2-morpholin-4-ylethyl (3-{[4- oxo-1-(1-propyl-1H-pyrazol-4- yl)-1,4-dihydropyridazin-3- yl]methyl}phenyl)carbamate | Calc'd 467, found 467 |
| 118 | 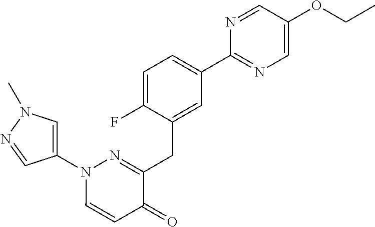 | 3-[5-(5-ethoxypyrimidin-2-yl)-2- fluorobenzyl]-1-(1-methyl-1H- pyrazol-4-yl)pyridazin-4(1H)-one | Calc'd 407, found 407 |
| 119 | 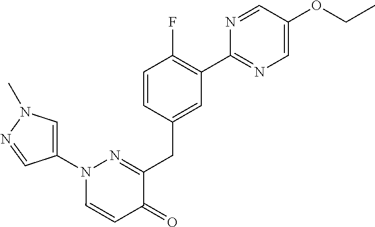 | 3-[3-(5-ethoxypyrimidin-2-yl)-4- fluorobenzyl]-1-(1-methyl-1H- pyrazol-4-yl)pyridazin-4(1H)-one | Calc'd 407, found 407 |
| 120 | 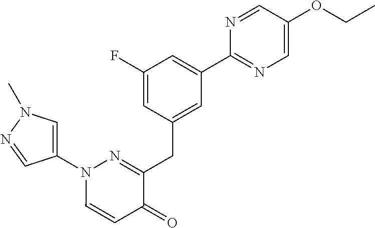 | 3-[3-(5-ethoxypyrimidin-2-yl)-5- fluorobenzyl]-1-(1-methyl-1H- pyrazol-4-yl)pyridazin-4(1H)-one | Calc'd 407, found 407 |
| 121 | 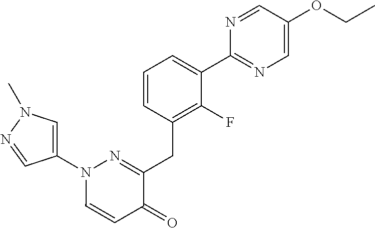 | 3-[3-(5-ethoxypyrimidin-2-yl)-2- fluorobenzyl]-1-(1-methyl-1H- pyrazol-4-yl)pyridazin-4(1H)-one | Calc'd 407, found 407 |
| 122 | 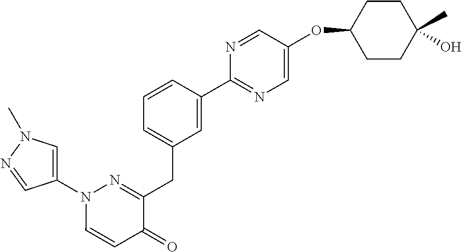 | 3-(3-{5-[(trans-4-hydroxy-4- methylcyclohexyl)oxy]pyrimidin- 2-yl}benzyl)-1-(1-methyl-1H- pyrazol-4-yl)pyridazin-4(1H)-one | Calc'd 473, found 473 |
| 123 | 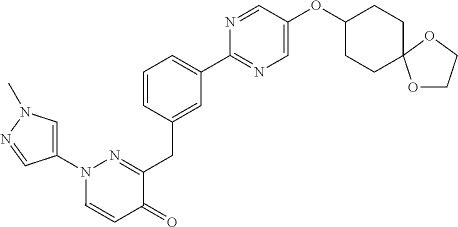 | 3-{3-[5-(1,4-dioxaspiro[4.5]dec- 8-yloxy)pyrimidin-2-yl]benzyl}- 1-(1-methyl-1H-pyrazol-4- yl)pyridazin-4(1H)-one | Calc'd 501, found 501 |
| 124 | 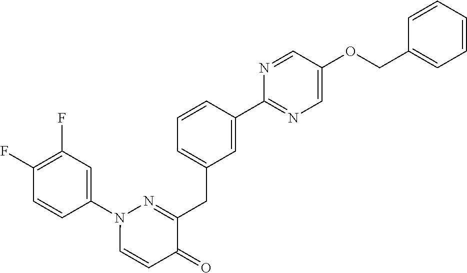 | 3-{3-[5-(benzyloxy)pyrimidin-2- yl]benzyl}-1-(3,4- difluorophenyl)pyridazin-4(1H)- one | Calc'd 483, found 483 |
| 125 | 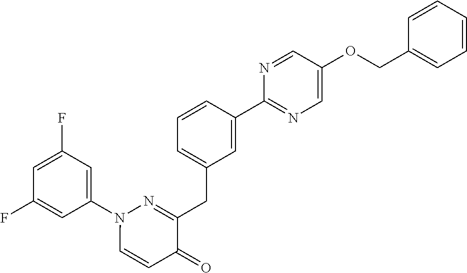 | 3-{3-[5-(benzyloxy)pyrimidin-2- yl]benzyl}-1-(3,5- difluorophenyl)pyridazin-4(1H)- one | Calc'd 483, found 483 |
| 126 | 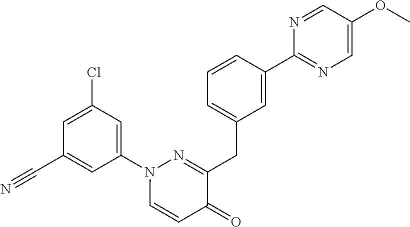 | 3-chloro-5-{3-[3-(5- methoxypyrimidin-2-yl)benzyl]- 4-oxopyridazin-1(4H)- yl}benzonitrile | Calc'd 430, found 430 |
| 127 | 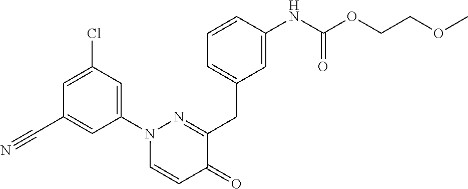 | 2-methoxyethyl (3-{[1-(3-chloro- 5-cyanophenyl)-4-oxo-1,4- dihydropyridazin-3- yl]methyl}phenyl)carbamate | Calc'd 439, found 439 |
| 128 | 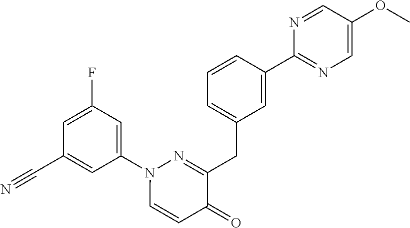 | 3-fluoro-5-{3-[3-(5- methoxypyrimidin-2-yl)benzyl]- 4-oxopyridazin-1(4H)- yl}benzonitrile | Calc'd 414, found 414 |
| 129 | 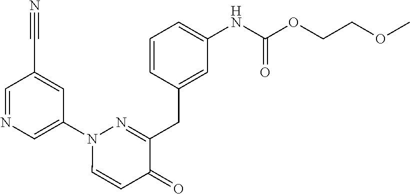 | 2-methoxyethyl (3-{[1-(5- cyanopyridin-3-yl)-4-oxo-1,4- dihydropyridazin-3- yl]methyl}phenyl)carbamate | Calc'd 406, found 406 |
| 130 | 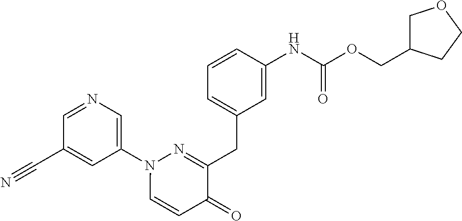 | rac-tetrahydrofuran-3-ylmethyl (3-{[1-(5-cyanopyridin-3-yl)-4- oxo-1,4-dihydropyridazin-3- yl]methyl}phenyl)carbamate | Calc'd 432, found 432 |
| 131 | 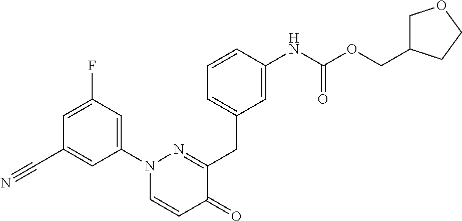 | rac-tetrahydrofuran-3-ylmethyl (3-{[1-(3-cyano-5-fluorophenyl)- 4-oxo-1,4-dihydropyridazin-3- yl]methyl}phenyl)carbamate | Calc'd 449, found 449 |
| 132 | 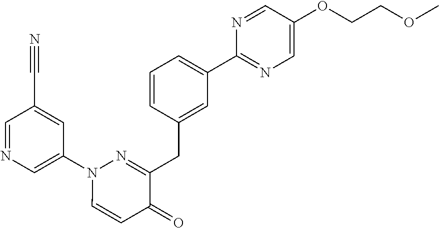 | 5-[3-{3-[5-(2- methoxyethoxy)pyrimidin-2- yl]benzyl}-4-oxopyridazin- 1(4H)-yl]pyridine-3-carbonitrile | Calc'd 441, found 441 |
| 133 | 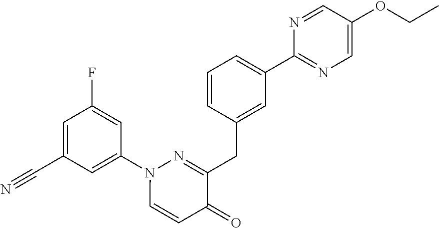 | 3-{3-[3-(5-ethoxypyrimidin-2- yl)benzyl]-4-oxopyridazin-1(4H)- yl}-5-fluorobenzonitrile | Calc'd 428, found 428 |
| 134 | 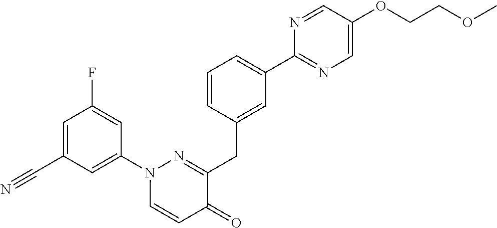 | 3-fluoro-5-[3-{3-[5-(2- methoxyethoxy)pyrimidin-2- yl]benzyl}-4-oxopyridazin- 1(4H)-yl]benzonitrile | Calc'd 458, found 458 |
| 135 | 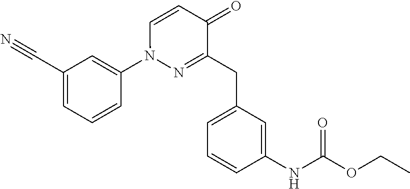 | ethyl (3-{[1-(3-cyanophenyl)-4- oxo-1,4-dihydropyridazin-3- yl]methyl}phenyl)carbamate | Calc'd 375, found 375 |
| 136 | 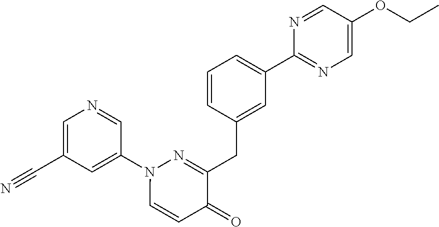 | 5-{3-[3-(5-ethoxypyrimidin-2- yl)benzyl]-4-oxopyridazin-1(4H)- yl}pyridine-3-carbonitrile | Calc'd 411, found 411 |
| 137 | 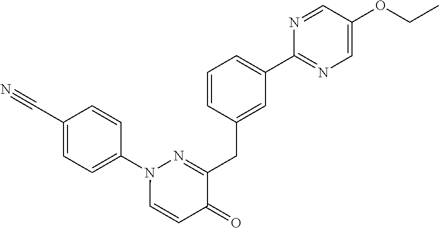 | 4-{3-[3-(5-ethoxypyrimidin-2- yl)benzyl]-4-oxopyridazin-1(4H)- yl}benzonitrile | Calc'd 410, found 410 |
| 138 | 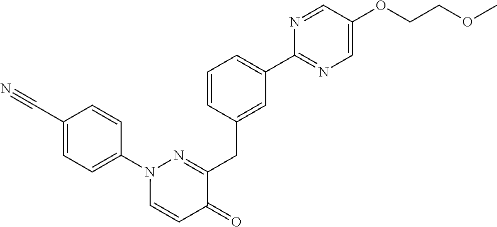 | 4-[3-{3-[5-(2- methoxyethoxy)pyrimidin-2- yl]benzyl}-4-oxopyridazin- 1(4H)-yl]benzonitrile | Calc'd 440, found 440 |
| 139 | 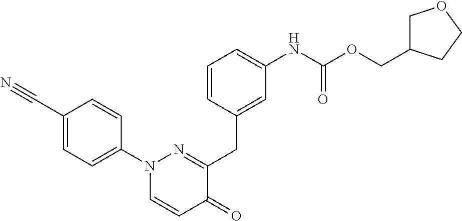 | rac-tetrahydrofuran-3-ylmethyl (3-{[1-(4-cyanophenyl)-4-oxo- 1,4-dihydropyridazin-3- yl]methyl}phenyl)carbamate | Calc'd 431, found 432 |
| 140 | 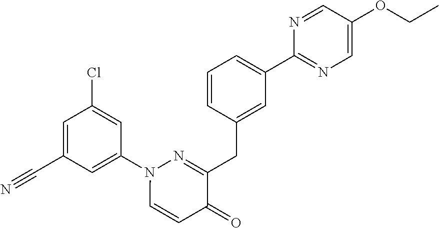 | 3-chloro-5-{3-[3-(5- ethoxypyrimidin-2-yl)benzyl]-4- oxopyridazin-1(4H)- yl}benzonitrile | Calc'd 444, found 444 |
| 141 | 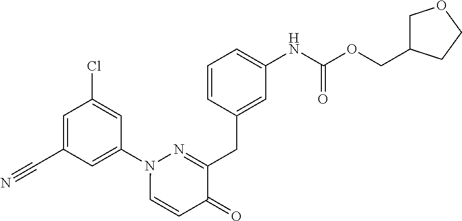 | rac-tetrahydrofuran-3-ylmethyl (3-{[1-(3-chloro-5-cyanophenyl)- 4-oxo-1,4-dihydropyridazin-3- yl]methyl}phenyl)carbamate | Calc'd 465, found 465 |
| 142 | 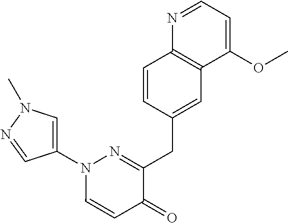 | 3-[(4-methoxyquinolin-6- yl)methyl]-1-(1-methyl-1H- pyrazol-4-yl)pyridazin-4(1H)-one | Calc'd 348, found 348 |
| 143 | 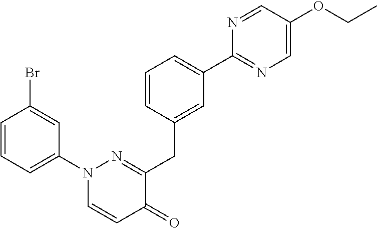 | 1-(3-bromophenyl)-3-[3-(5- ethoxypyrimidin-2- yl)benzyl]pyridazin-4(1H)-one | Calc'd 463, found 463 |
| 144 | 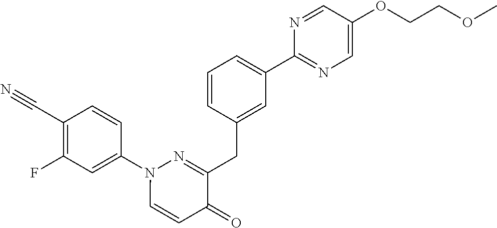 | 2-fluoro-4-[3-{3-[5-(2- methoxyethoxy)pyrimidin-2- yl]benzyl}-4-oxopyridazin- 1(4H)-yl]benzonitrile | Calc'd 458, found 458 |
| 145 | 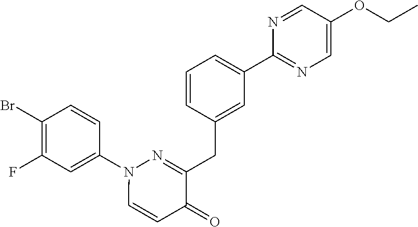 | 1-(4-bromo-3-fluorophenyl)-3-[3- (5-ethoxypyrimidin-2- yl)benzyl]pyridazin-4(1H)-one | Calc'd 481, found 481 |
| 146 | 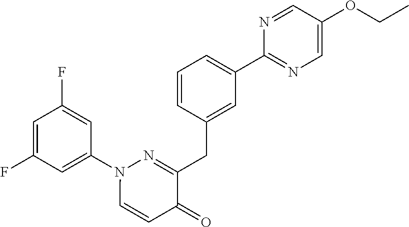 | 1-(3,5-difluorophenyl)-3-[3-(5- ethoxypyrimidin-2- yl)benzyl]pyridazin-4(1H)-one | Calc'd 421, found 421 |
| 147 | 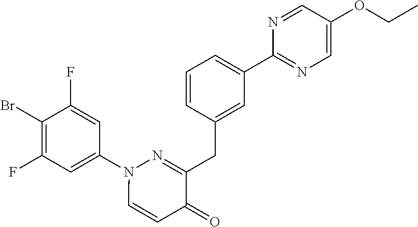 | 1-(4-bromo-3,5-difluorophenyl)- 3-[3-(5-ethoxypyrimidin-2- yl)benzyl]pyridazin-4(1H)-one | Calc'd 499, found 499 |
| 148 | 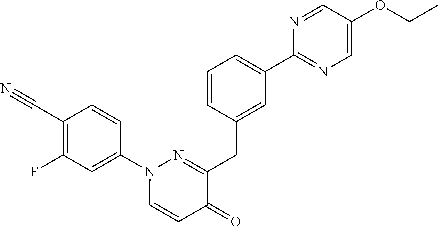 | 4-{3-[3-(5-ethoxypyrimidin-2- yl)benzyl]-4-oxopyridazin-1(4H)- yl}-2-fluorobenzonitrile | Calc'd 428, found 428 |
| 149 | 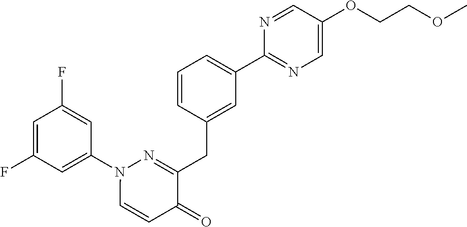 | 1-(3,5-difluorophenyl)-3-{3-[5- (2-methoxyethoxy)pyrimidin-2- yl]benzyl}pyridazin-4(1H)-one | Calc'd 451, found 451 |
| 150 | 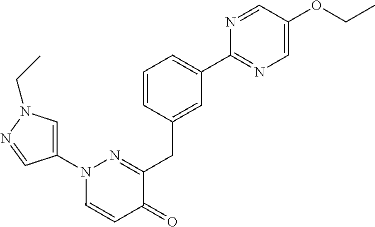 | 3-[3-(5-ethoxypyrimidin-2- yl)benzyl]-1-(1-ethyl-1H- pyrazol-4-yl)pyridazin-4(1H)-one | Calc'd 403, found 403 |
| 151 | 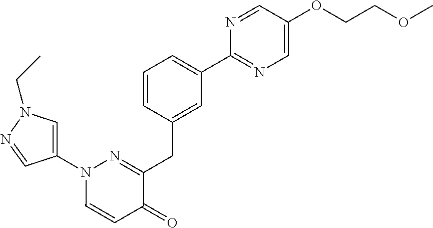 | 1-(1-ethyl-1H-pyrazol-4-yl)-3- {3-[5-(2- methoxyethoxy)pyrimidin-2- yl]benzyl}pyridazin-4(1H)-one | Calc'd 433, found 433 |
| 152 | 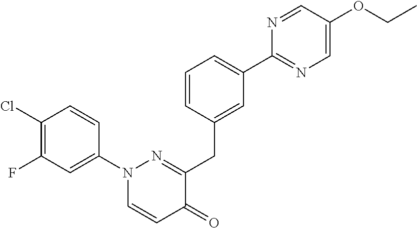 | 1-(4-chloro-3-fluorophenyl)-3-[3- (5-ethoxypyrimidin-2- yl)benzyl]pyridazin-4(1H)-one | Calc'd 437, found 437 |
| 153 | 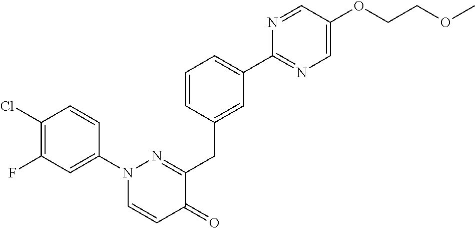 | 1-(4-chloro-3-fluorophenyl)-3- {3-[5-(2- methoxyethoxy)pyrimidin-2- yl]benzyl}pyridazin-4(1H)-one | Calc'd 467, found 467 |
| 154 | 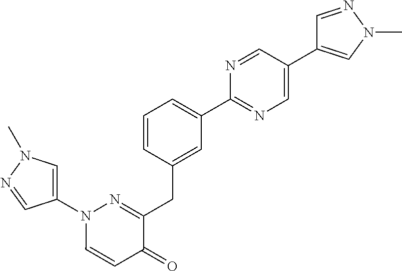 | 1-(1-methyl-1H-pyrazol-4-yl)-3- {3-[5-(1-methyl-1H-pyrazol-4- yl)pyrimidin-2- yl]benzyl}pyridazin-4(1H)-one | Calc'd 415, found 415 |
| 155 | 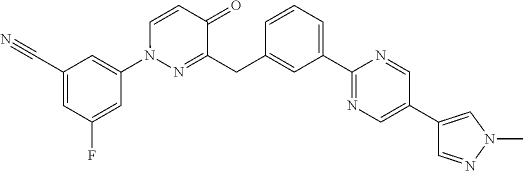 | 3-fluoro-5-[3-{3-[5-(1-methyl- 1H-pyrazol-4-yl)pyrimidin-2- yl]benzyl}-4-oxopyridazin- 1(4H)-yl]benzonitrile | Calc'd 464, found 464 |
| 156 | 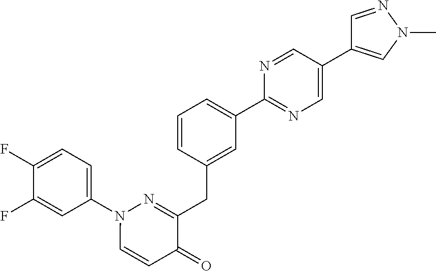 | 1-(3,4-difluorophenyl)-3-{3-[5- (1-methyl-1H-pyrazol-4- yl)pyrimidin-2- yl]benzyl}pyridazin-4(1H)-one | Calc'd 457, found 457 |
| 157 | 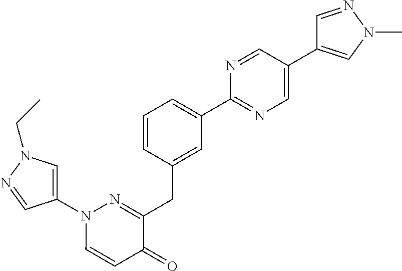 | 1-(1-ethyl-1H-pyrazol-4-yl)-3- {3-[5-(1-methyl-1H-pyrazol-4- yl)pyrimidin-2- yl]benzyl}pyridazin-4(1H)-one | Calc'd 439, found 439 |
| 158 | 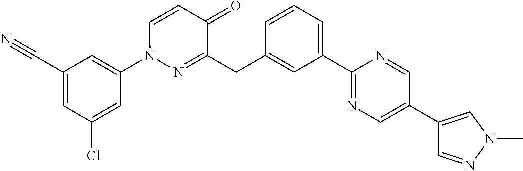 | 3-chloro-5-[3-{3-[5-(1-methyl- 1H-pyrazol-4-yl)pyrimidin-2- yl]benzyl}-4-oxopyridazin- 1(4H)-yl]benzonitrile | Calc'd 480, found 480 |
| 159 | 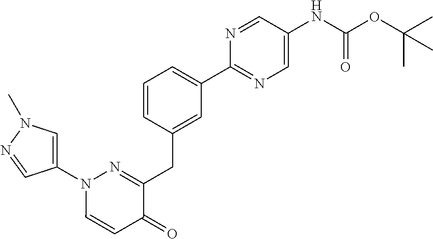 | tert-butyl [2-(3-{[1-(1-methyl- 1H-pyrazol-4-yl)-4-oxo-1,4- dihydropyridazin-3- yl]methyl}phenyl)pyrimidin-5- yl]carbamate | Calc'd 460, found 460 |
| 160 | 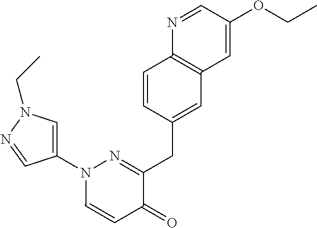 | 3-[(3-ethoxyquinolin-6- yl)methyl]-1-(1-ethyl-1H- pyrazol-4-yl)pyridazin-4(1H)-one | Calc'd 376, found 376 |
| 161 | 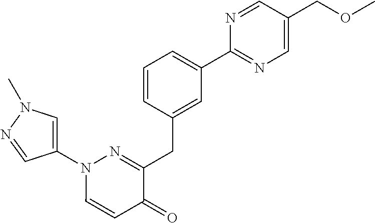 | 3-{3-[5- (methoxymethyl)pyrimidin-2- yl]benzyl}-1-(1-methyl-1H- pyrazol-4-yl)pyridazin-4(1H)-one | Calc'd 389, found 389 |
| 162 | 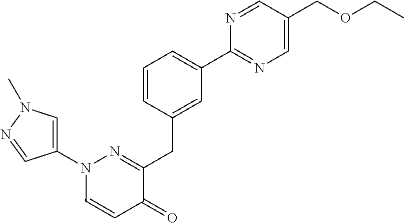 | 3-{3-[5- (ethoxymethyl)pyrimidin-2- yl]benzyl}-1-(1-methyl-1H- pyrazol-4-yl)pyridazin-4(1H)-one | Calc'd 403, found 403 |
| 163 | 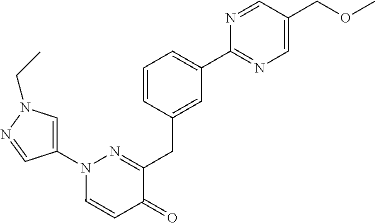 | 1-(1-ethyl-1H-pyrazol-4-yl)-3- {3-[5- (methoxymethyl)pyrimidin-2- yl]benzyl}pyridazin-4(1H)-one | Calc'd 403, found 403 |
| 164 | 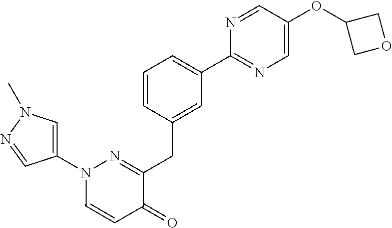 | 1-(1-methyl-1H-pyrazol-4-yl)-3- {3-[5-(oxetan-3-yloxy)pyrimidin- 2-yl]benzyl}pyridazin-4(1H)-one | Calc'd 417, found 417 |
| 165 | 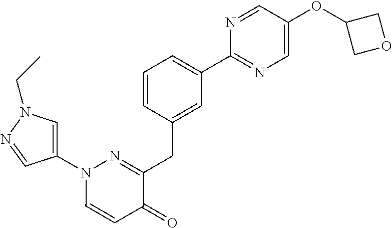 | 1-(1-ethyl-1H-pyrazol-4-yl)-3- {3-[5-(oxetan-3-yloxy)pyrimidin- 2-yl]benzyl}pyridazin-4(1H)-one | Calc'd 431, found 431 |
| 166 | 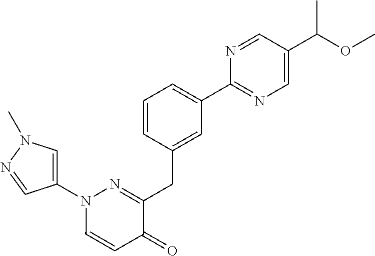 | rac-3-{3-[5-(1-methoxyethyl) pyrimidin-2-yl]benzyl}-1-(1- methyl-1H-pyrazol-4- yl)pyridazin-4(1H)-one | Calc'd 403, found 403 |
| 167 | 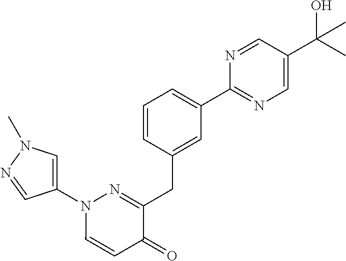 | 3-{3-[5-(1-hydroxy-1- methylethyl)pyrimidin-2- yl]benzyl}-1-(1-methyl-1H- pyrazol-4-yl)pyridazin-4(1H)-one | Calc'd 403, found 403 |
| 168 | 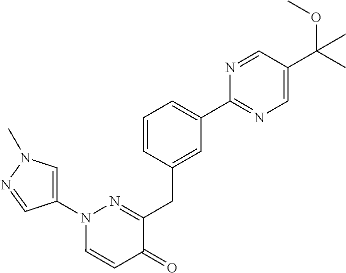 | 3-{3-[5-(1-methoxy-1- methylethyl)pyrimidin-2- yl]benzyl}-1-(1-methyl-1H- pyrazol-4-yl)pyridazin-4(1H)-one | Calc'd 417, found 417 |
| 169 | 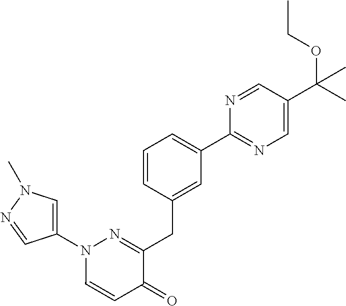 | 3-{3-[5-(1-ethoxy-1- methylethyl)pyrimidin-2- yl]benzyl}-1-(1-methyl-1H- pyrazol-4-yl)pyridazin-4(1H)-one | Calc'd 431, found 431 |
| 170 | 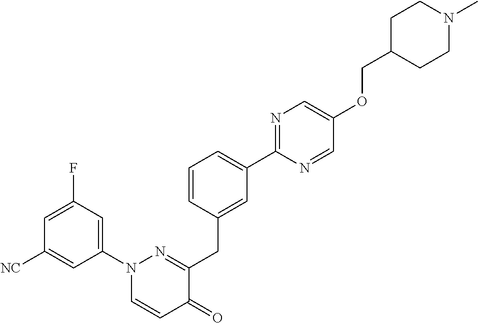 | 3-fluoro-5-[3-(3-{5-[(1- methylpiperidin-4- yl)methoxy]pyrimidin-2- yl}benzyl)-4-oxopyridazin- 1(4H)-yl]benzonitrile | Calc'd 511, found 511 |
| 171 | 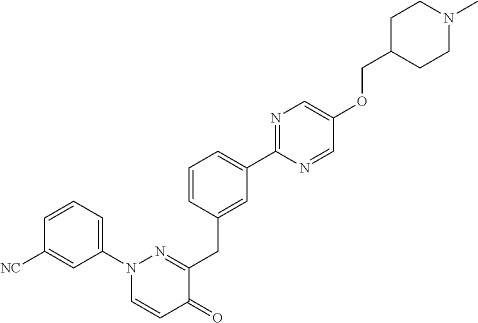 | 3-[3-(3-{5-[(1-methylpiperidin-4- yl)methoxy]pyrimidin-2- yl}benzyl)-4-oxopyridazin- 1(4H)-yl]benzonitrile | Calc'd 493, found 493 |
| 172 | 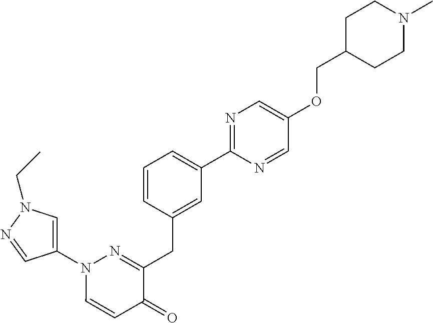 | 1-(1-ethyl-1H-pyrazol-4-yl)-3-(3- {5-[(1-methylpiperidin-4- yl)methoxy]pyrimidin-2- yl}benzyl)pyridazin-4(1H)-one | Calc'd 486, found 486 |
| 173 | 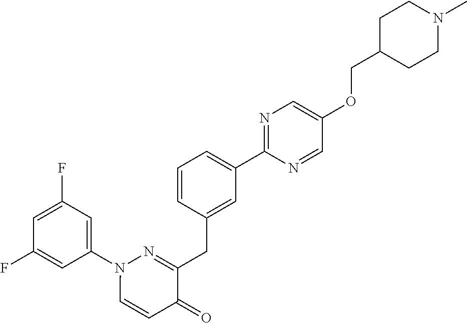 | 1-(3,5-difluorophenyl)-3-(3-{5- [(1-methylpiperidin-4- yl)methoxy]pyrimidin-2- yl}benzyl)pyridazin-4(1H)-one | Calc'd 504, found 504 |
| 174 | 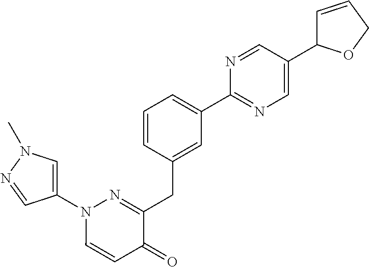 | rac-3-{3-[5-(2,5-Dihydrofuran-2- yl)pyrimidin-2-yl]benzyl}-1-(1- methyl-1H-pyrazol-4- yl)pyridazin-4(1H)-one | Calc'd 413, found 413 |
| 175 | 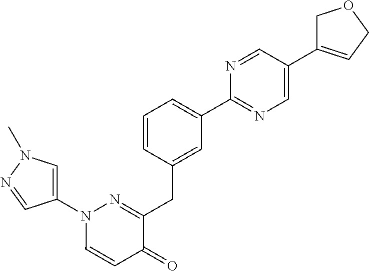 | 3-{3-[5-(2,5-dihydrofuran-3- yl)pyrimidin-2-yl]benzyl}-1-(1- methyl-1H-pyrazol-4- yl)pyridazin-4(1H)-one | Calc'd 413, found 413 |
| 176 | 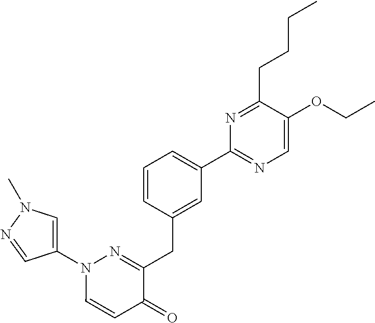 | 3-[3-(4-butyl-5-ethoxypyrimidin- 2-yl)benzyl]-1-(1-methyl-1H- pyrazol-4-yl)pyridazin-4(1H)-one | Calc'd 445, found 445 |
| 177 | 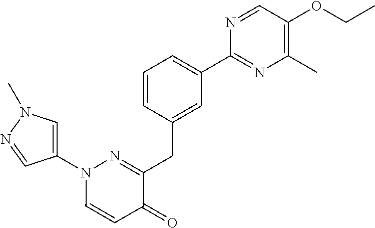 | 3-[3-(5-ethoxy-4- methylpyrimidin-2-yl)benzyl]-1- (1-methyl-1H-pyrazol-4- yl)pyridazin-4(1H)-one | Calc'd 403, found 403 |
| 178 | 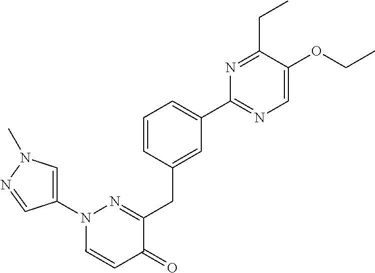 | 3-[3-(5-ethoxy-4-ethylpyrimidin- 2-yl)benzyl]-1-(1-methyl-1H- pyrazol-4-yl)pyridazin-4(1H)-one | Calc'd 417, found 417 |

The following Intermediates were prepared from Intermediates #1-4, 8-10, 19-20, 38-39 and #42-43, 62, 83, 85-86, 88, 104 (and commercial aryl boronates) according to Scheme 4 following similar procedures described for Examples #1-6, which can be achieved by those of ordinary skill in the art of organic synthesis.

| | | | Exact |
|---|---|---|---|
| | | IUPAC | Mass |
| Intermediate | Structure | Name | [M + H]⁺ |
| 106 | 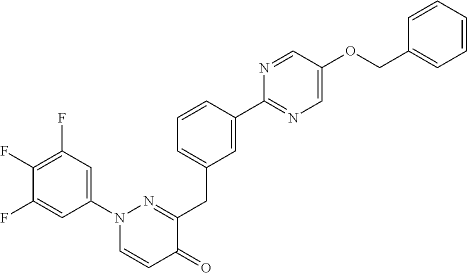 | 3-{3-[5- (benzyloxy) pyrimidin-2-yl] benzyl}-1-(3,4,5- trifluorophenyl) pyridazin- 4(1H)-one | Calc′d 501, found 501 |
| 107 | 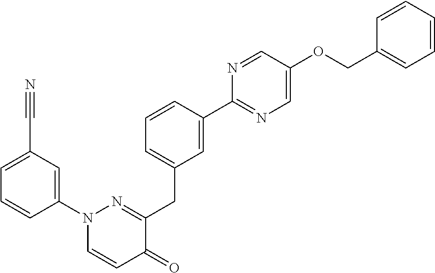 | 3-[3-{3-[5- (benzyloxy) pyrimidin-2- yl]benzyl}-4- oxopyridazin- 1(4H)-yl] benzonitrile | nd |
| 108 | 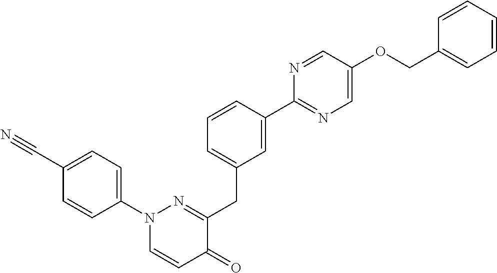 | 4-[3-{3-[5- (benzyloxy) pyrimidin-2- yl]benzyl}-4- oxopyridazin- 1(4H)-yl] benzonitrile | Calc′d 472, found 472 |
| 109 | 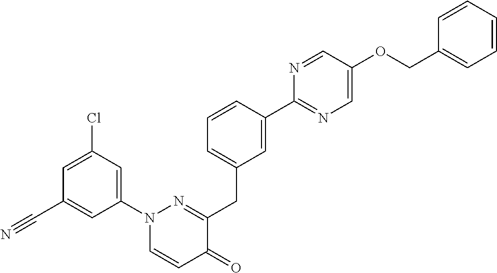 | 3-[3-{3-[5- (benzyloxy) pyrimidin-2- yl]benzyl}-4- oxopyridazin- 1(4H-yl]-5- chlorobenzonitrile | Calc′d 506, found 506 |
| 110 | 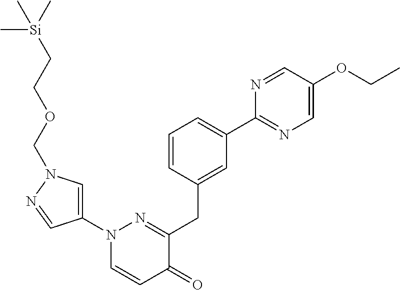 | 3-[3-(5- ethoxypyrimidin- 2-yl)benzyl]- 1-(1-{[2- (trimethylsilyl) ethoxy]methyl}- 1H-pyrazol-4- yl)pyridazin- 4(1H)-one | Calc′d 505, found 505 |
| 111 | 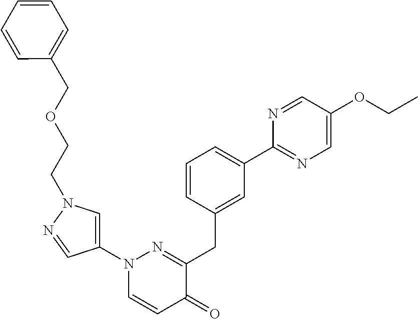 | 1-{1-[2- (benzyloxy) ethyl]-1H- pyrazol-4-yl}- 3-[3-(5- ethoxypyrimidin- 2-yl)benzyl] pyridazin- 4(1H)-one | Calc′d 509, found 509 |
| 112 | 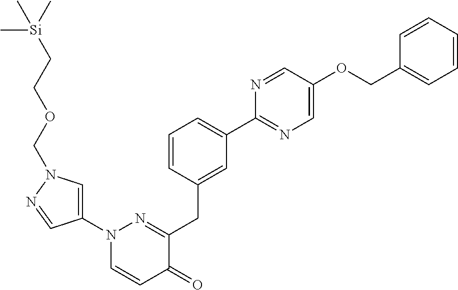 | 3-{3-[5- (benzyloxy) pyrimidin-2- yl]benzyl}- 1-(1-{[2- (trimethylsilyl) ethoxy]methyl}- 1H-pyrazol-4- yl)pyridazin- 4(1H)-one | Calc′d 567, found 567 |
| 113 | 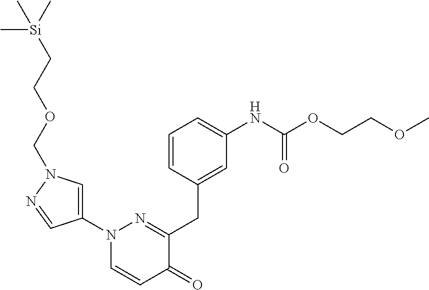 | 2-methoxyethyl (3-{[4-oxo- 1-(1-{[2- (trimethylsilyl) ethoxy]methyl}- 1H-pyrazol- 4-yl)-1,4- dihydropyridazin- 3-yl]methyl} phenyl)carbamate | Calc′d 500, found 500 |
| 114 | 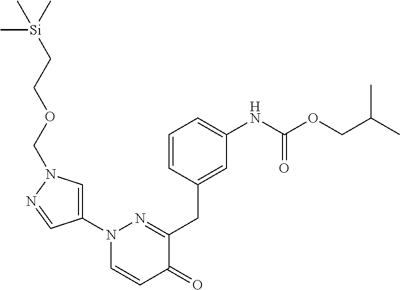 | 2-methylpropyl (3-{[4-oxo- 1-(1-{[2- (trimethylsilyl) ethoxy]methyl}- 1H-pyrazol- 4-yl)-1,4- dihydropyridazin- 3-yl]methyl} phenyl)carbamate | Calc′d 498, found 498 |
| 115 | 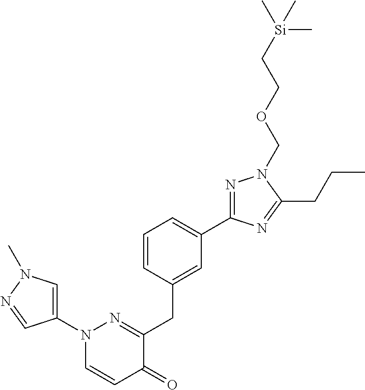 | 1-(1-methyl- 1H-pyrazol-4- yl)-3-[3-(5- propyl-1-{[2- (trimethylsilyl) ethoxy]methyl}- 1H-1,2,4-triazol- 3-yl)benzyl] pyridazin-4 (1H)-one | Calc′d 506, found 506 |
| 116 | 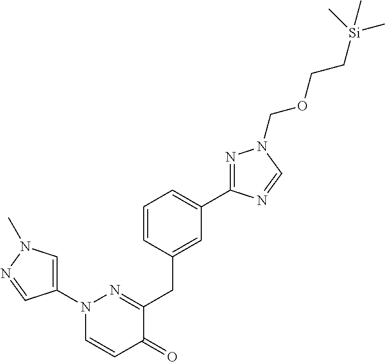 | 1-(1-methyl- 1H-pyrazol-4- yl)-3-[3-(1-{[2- (trimethylsilyl) ethoxy]methyl}- 1H-1,2,4-triazol- 3-yl)benzyl] pyridazin- 4(1H)-one | Calc′d 464, found 464 |
| 117 | 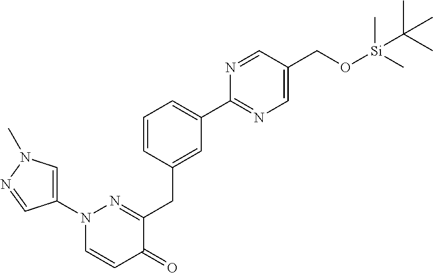 | 3-{3-[5-({[tert- butyl(dimethyl) silyl]oxy}methyl) pyrimidin- 2-yl]benzyl}- 1-(1-methyl- 1H-pyrazol-4- yl)pyridazin- 4(1H)-one | Calc′d 489, found 489 |
| 118 | 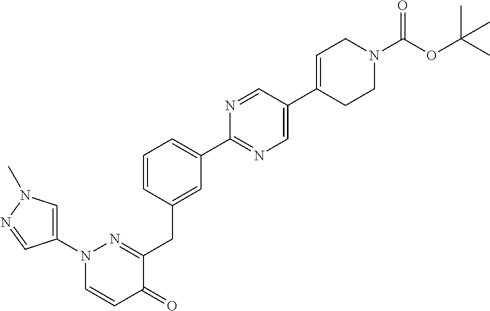 | tert-butyl 4- [2-(3-{[1-(1- methyl-1H- pyrazol-4-yl)- 4-oxo-1,4- dihydropyridazin- 3-yl]methyl} phenyl)pyrimidin- 5-yl]-3,6- dihydropyridine- 1(2H)-carboxylate | Calc′d 526, found 526 |
| 119 | 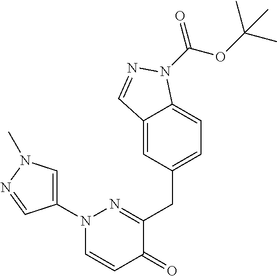 | tert-butyl 5-{[1- (1-methyl-1H- pyrazol-4-yl)- 4-oxo-1,4- dihydropyridazin- 3-yl]methyl}- 1H-indazole-1- carboxylate | Calc′d 407, found 407 |
| 120 | 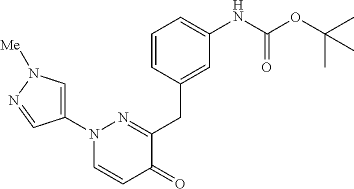 | tert-butyl (3-{[1- (1-methyl-1H- pyrazol-4-yl)- 4-oxo-1,4- dihydropyridazin- 3-yl]methyl} phenyl)carbamate | Calc′d 382, found 382 |
| 121 | 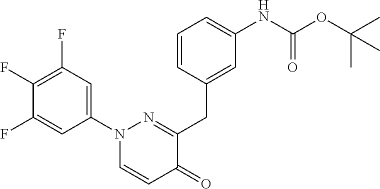 | tert-butyl (3-{[4- oxo-1-(3,4,5- trifluorophenyl)- 1,4- dihydropyridazin- 3-yl]methyl} phenyl)carbamate | Calc′d 432, found 432 |
| 122 | 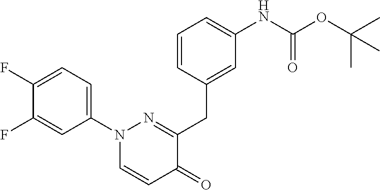 | tert-butyl (3- {[1-(3,4- difluorophenyl)- 4-oxo-1,4- dihydropyridazin- 3-yl]methyl} phenyl)carbamate | Calc′d 414, found 414 |
| 123 | 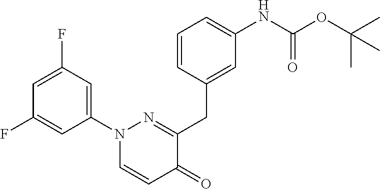 | tert-butyl (3- {[1-(3,5- difluorophenyl)- 4-oxo-1,4- dihydropyridazin- 3-yl]methyl} phenyl)carbamate | Calc′d 414, found 414 |
| 124 | 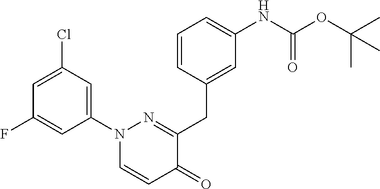 | tert-butyl (3- {[1-(3-chloro- 5-fluorophenyl)- 4-oxo-1,4- dihydropyridazin- 3-yl]methyl} phenyl)carbamate | Calc′d 430, found 430 |
| 125 | 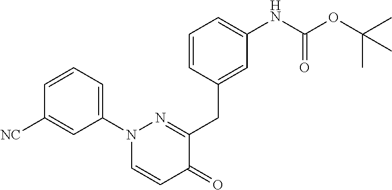 | tert-butyl (3- {[1-(3- cyanophenyl)- 4-oxo-1,4- dihydropyridazin- 3-yl]methyl} phenyl)carbamate | Calc′d 403, found 403 |
| 126 | 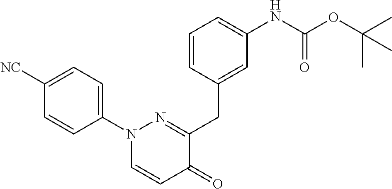 | tert-butyl (3- {[1-(4- cyanophenyl)- 4-oxo-1,4- dihydropyridazin- 3-yl]methyl} phenyl)carbamate | Calc′d 403, found 403 |
| 127 | 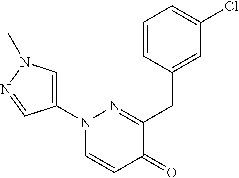 | 3-(3- chlorobenzyl)- 1-(1-methyl- 1H-pyrazol-4- yl)pyridazin- 4(1H)-one | Calc′d 301, found 301 |
| 128 | 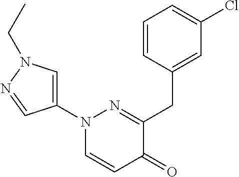 | 3-(3- chlorobenzyl)- 1-(1-ethyl-1H- pyrazol-4-yl) pyridazin- 4(1H)-one | Calc′d 315, found 315 |
| 129 | 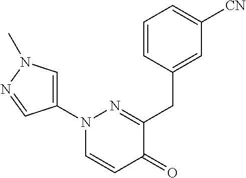 | 3-{[1-(1- methyl-1H- pyrazol-4-yl)- 4-oxo-1,4- dihydropyridazin- 3-yl]methyl} benzonitrile | Calc′d 292, found 292 |
| 130 | 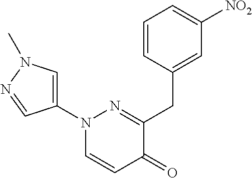 | 1-(1-methyl- 1H-pyrazol- 4-yl)-3-(3- nitrobenzyl) pyridazin- 4(1H)-one | Calc′d 312, found 312 |
| 131 | 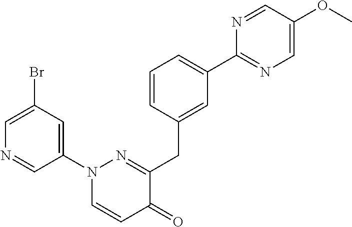 | 1-(5- bromopyridin- 3-yl)-3-[3-(5- methoxypyrimidin- 2-yl)benzyl] pyridazin- 4(1H)-one | Calc′d 450, found 450 |

### Scheme 4

### Examples #179 and 180

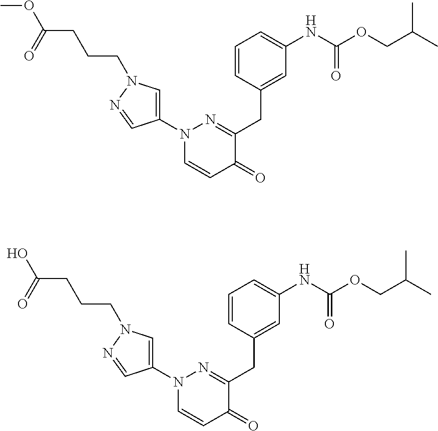

### Methyl 4-{4-[3-(3-{[(2-Methylpropoxy)carbonyl]amino}benzyl)-4-oxopyridazin-1(4H)-yl]-1H-pyrazol-1-yl}butanoate and 4-{4-[3-(3-{[(2-methylpropoxy)carbonyl]amino}benzyl)-4-oxopyridazin-1(4H)-yl]-1H-pyrazol-1-yl}butanoic acid

### Step 1. Methyl 4-{4-[3-(3-{[(2-methylpropoxy)carbonyl]amino}benzyl)-4-oxopyridazin-1(4H)-yl]-1H-pyrazol-1-yl}butanoate and 4-{4-[3-(3-{[(2-methylpropoxy)carbonyl]amino}benzyl)-4-oxopyridazin-1(4H)-yl]-1H-pyrazol-1-yl}butanoic acid

A mixture of methyl 4-{4-[3-(3-{[(2-methylpropoxy)carbonyl]amino}benzyl)-4-oxopyridazin-1(4H)-yl]-1H-pyrazol-1-yl}butanoate and 4-{4-[3-(3-{[(2-Methylpropoxy)carbonyl]amino}benzyl)-4-oxopyridazin-1(4H)-yl]-1H-pyrazol-1-yl}butanoic acid was prepared from methyl 4-{4-[3-(chloromethyl)-4-oxopyridazin-1(4H)-yl]-1H-pyrazol-1-yl}butanoate (Intermediate #28) and isobutyl [3-(4,4,5,5-tetramethyl-1,3,2-dioxaborolan-2-yl)phenyl]carbamate (Intermediate #42) according to the procedure described for ethyl(3-{[4-oxo-1-(3,4,5-trifluorophenyl)-1,4-dihydropyridazin-3-yl]methyl}phenyl)carbamate (Example #2). The products were separated by diluting the reaction mixture with EtOAc and basifying with NaHCO₃ (pH 10). The layers were separated, the organic phase was dried over Na₂SO₄, concentrated in vacuo and the residue purified by flash chromatography (MPLC MeOH-DCM) to afford methyl 4-{4-[3-(3-{[(2-methylpropoxy)carbonyl]amino}benzyl)-4-oxopyridazin-1(4H)-yl]-1H-pyrazol-1-yl}butanoate (Example #179). The aqueous layer was acidified with 2N HCl (pH 4), extracted with 3:1 chloroform/IPA (3×) and the combined organic phases were concentrated in vacuo to afford 4-{4-[3-(3-{[(2-methylpropoxy)carbonyl]amino}benzyl)-4-oxopyridazin-1(4H)-yl]-1H-pyrazol-1-yl}butanoic acid (Example #180).

### Example #179

LRMS (ESI) calc'd for C24H30N5O5 [M+H]⁺: 468. Found: 468.

### Example #180

LRMS-(ESI) calc'd for C23H28N5O5 [M+H]⁺: 454. Found: 454.

The following examples were prepared from Intermediates #29-30 and Intermediate #42 according to Scheme 4 following a similar procedure described for Example #180, which can be achieved by those of ordinary skill in the art of organic synthesis.

| | | | Exact |
|---|---|---|---|
| | | | Mass |
| Example | Structure | IUPAC Name | [M + H]+ |
| 181 | 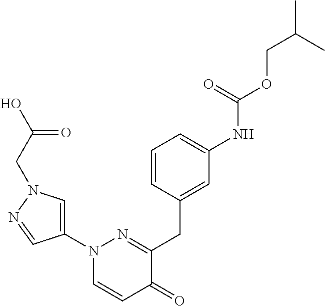 | {4-[3-(3-{[(2- methylpropoxy) carbonyl]amino}benzyl)- 4-oxopyridazin- 1(4H)-yl]-1H- pyrazol-1-yl}acetic acid | Calc'd 426, found 426 |
| 182 | 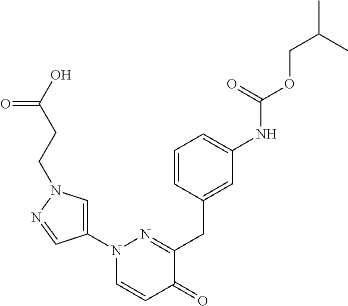 | 3-{4-[3-(3-{[(2- methylpropoxy) carbonyl]amino}benzyl)- 4-oxopyridazin- 1(4H)-yl]-1H- pyrazol-1- yl}propanoic acid | Calc'd 440, found 440 |

### Scheme 4

### Example #183

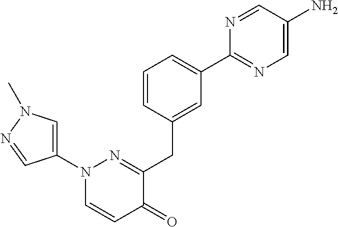

### 3-[3-(5-Aminopyrimidin-2-yl)benzyl]-1-(1-methyl-1H-pyrazol-4-yl)pyridazin-4(1H)-one

### Step 1. 3-[3-(5-Aminopyrimidin-2-yl)benzyl]-(1-methyl-1H-pyrazol-4-ylpyridazin-4(1H)-one

tert-Butyl [2-(3-{[1-(1-methyl-1H-pyrazol-4-yl)-4-oxo-1,4-dihydropyridazin-3-yl]methyl}phenyl)pyrimidin-5-yl]carbamate (Example #159, 910 mg, 1.98 mmol) was dissolved in DCM (990 μL), TFA (990 μL) was added and the reaction mixture was stirred for 4-hours. The solvent was removed in vacuo, aqueous NaHCO₃ was added and the products extracted into 4:1 DCM-MeOH (3×). The combined organic extracts were concentrated in vacuo to provide 3-[3-(5-aminopyrimidin-2-yl)benzyl]-1-(1-methyl-1H-pyrazol-4-yl)pyridazin-4(1H)-one.

LRMS (ESI) calc'd for C19H18N7O [M+H]⁺: 460. Found: 460.

### Scheme 4

### Example #184

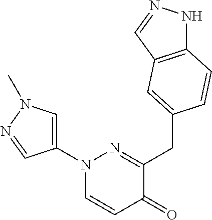

### 3-(1H-Indazol-5-ylmethyl)-1-(1-methyl-1H-pyrazol-4-yl)pyridazin-4(1H)-one

### Step 1. 3-(1H-Indazol-5-ylmethyl)-1-(1-methyl-1H-pyrazol-4-yl)pyridazin-4(1H-one

tert-Butyl 5-{[1-(1-methyl-1H-pyrazol-4-yl)-4-oxo-1,4-dihydropyridazin-3-yl]methyl}-1H-indazole-1-carboxylate (Intermediate #119, 77 mg, 0.19 mmol) was dissolved in DCM (0.5 mL) then TFA (0.5 mL) was added. The reaction mixture stirred at r.t. for 30 min, then concentrated in vacuo, diluted with MeCN (2 mL) and water (1 mL) and purified by reverse phase preparative HPLC (2-50% MeCN—H₂O, 0.05% TFA). The fractions containing the pure product were filtered through a PL-HCO₃ cartridge (Stratospheres™, 0.9 mmol) and lyophilized to afford 3-(1H-indazol-5-ylmethyl)-1-(1-methyl-1H-pyrazol-4-yl)pyridazin-4(1H)-one as a white solid.

LRMS (ESI) calc'd for C16H15N6O [M+H]⁺: 307. Found: 307.

### Scheme 4

### Intermediate #132

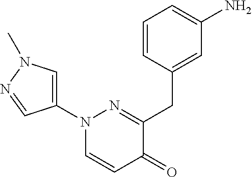

### 3-(3-Aminobenzyl)-1-(1-methyl-1H-pyrazol-4-yl)pyridazin-4(1H)-one

### Step 1. 3-(3-Aminobenzyl)-1-(1-methyl-1H-pyrazol-4-yl)pyridazin-4(1H)-one

tert-Butyl (3-{[1-(1-methyl-1H-pyrazol-4-yl)-4-oxo-1,4-dihydropyridazin-3-yl]methyl}phenyl)carbamate (Intermediate #120, 2.35 g, 6.15 mmol) was stirred in DCM (60 mL)/TFA (6 mL) at r.t. overnight. The solvent was removed in vacuo and the residue purified by flash chromatography (MPLC, [0-15% (1% NH₄OH-MeOH)-DCM]). The residue from the combined product fractions was partitioned between saturated NaHCO₃ and MeOH-DCM. The aqueous phase was extracted with further portions of MeOH-DCM (2×). The combined organic extracts were dried over MgSO₄, filtered and concentrated in vacuo to give 3-(3-aminobenzyl)-1-(1-methyl-1H-pyrazol-4-yl)pyridazin-4(1H)-one as a white solid.

LRMS (ESI) calc'd for C15H16N5O [M+H]⁺: 282. Found: 282.

The following intermediates were prepared from Intermediates #121-126 according to Scheme 4 following similar procedures described for Intermediate #132, which can be achieved by those of ordinary skill in the art of organic synthesis.

| | | | Exact |
|---|---|---|---|
| | | | Mass |
| Intermediate | Structure | IUPAC Name | [M+ H]+ |
| 133 | 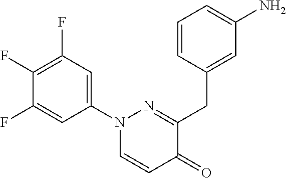 | 3-(3-aminobenzyl)-1- (3,4,5-trifluorophenyl) pyridazin-4(1H)-one | Calc'd 332, found 332 |
| 134 | 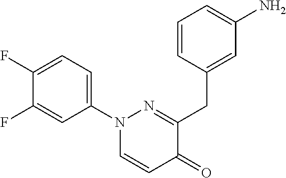 | 3-(3-aminobenzyl)-1- (3,4-difluorophenyl) pyridazin-4(1H)-one | Calc'd 314, found 314 |
| 135 | 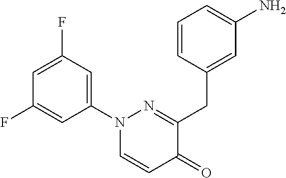 | 3-(3-aminobenzyl)-1- (3,5-difluorophenyl) pyridazin-4(1H)-one | Calc'd 314, found 314 |
| 136 | 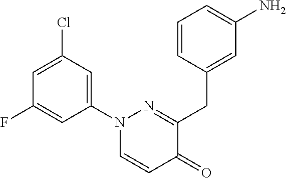 | 3-(3-aminobenzyl)-1- (3-chloro-5-fluoro- phenyl)pyridazin- 4(1H)-one | Calc'd 330, found 330 |
| 137 | 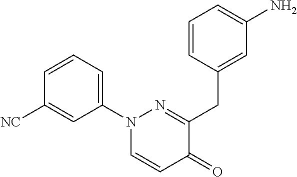 | 3-[3-(3-aminobenzyl)- 4-oxopyridazin-1(4H)- yl]benzonitrile | Calc'd 303, found 303 |
| 138 | 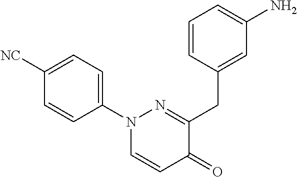 | 4-[3-(3-aminobenzyl)-4- oxopyridazin-1(4H)- yl]benzonitrile | Calc'd 303, found 303 |

### Scheme 4

### Example #185

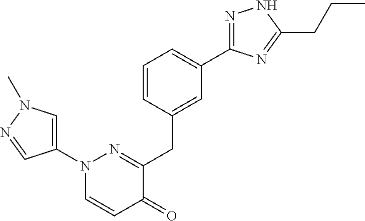

### 1-(1-Methyl-1H-pyrazol-4-yl)-3-[3-(5-propyl-1H-1,2,4-triazol-3-yl)benzyl]pyridazin-4(1H)-one

### Step 1. 1-(1-Methyl-1H-pyrazol-4-yl)-3-[3-(5-propyl-1H-1,2,4-triazol-3-yl)benzyl]pyridazin-4(1H)-one

1-(1-Methyl-1H-pyrazol-4-yl)-3-[3-(5-propyl-1-{[2-(trimethylsilyl)ethoxy]methyl}-1H-1,2,4-triazol-3-yl)benzyl]pyridazin-4(1H)-one (Intermediate #115, 30-mg, 0.059 mmol) was dissolved in EtOH (1.5 mL) and 2 N HCl (1.5 mL) was added. The reaction mixture was stirred at 80° C. overnight. Room temperature was attained and the solvent removed in vacuo to give the HCl salt of 1-(1-methyl-1H-pyrazol-4-yl)-3-[3-(5-propyl-1H-1,2,4-triazol-3-yl)benzyl]pyridazin-4(1H)-one.

LRMS (ESI) calc'd for C20H23ClN7O [M+H]⁺: 376. found 376.

### Scheme 4

### Example #186

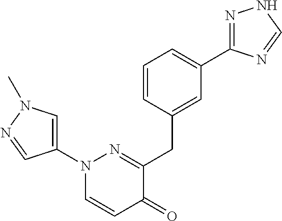

### 1-(1-Methyl-1H-pyrazol-4-yl)-3-[3-(1H-1,2,4-triazol-3-yl)benzyl]pyridazin-4(1H)-one

### Step 1. 1-(1-Methyl-1H-pyrazol-4-yl)-3-[3-(1H-1,2,4-triazol-3-yl)benzyl]pyridazin-4(1H)-one

1-(1-Methyl-1H-pyrazol-4-yl)-3-[3-(1-{[2-(trimethylsilyl)ethoxy]methyl}-1H-1,2,4-triazol-3-yl)benzyl]pyridazin-4(1H)-one (Intermediate #116, 35 mg, 0.075 mmol) was stirred in EtOH (0.4 mL)/2 N HCl (0.4 mL) at 80° C. for 4 hours. Room temperature was attained and the solvent removed in vacuo. The residue was purified by reverse phase preparative HPLC (10-75% MeCN—H₂O, 0.05% TFA) to give the TFA salt of 1-(1-methyl-1H-pyrazol-4-yl)-3-[3-(1H-1,2,4-triazol-3-yl)benzyl]pyridazin-4(1H)-one as an orange solid.

LRMS (ESI) calc'd for C17H16N7O [M+H]⁺: 334. found 334.

### Scheme 4

### Example #187

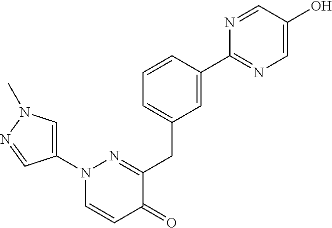

### 3-[3-(5-Hydroxypyrimidin-2-yl)benzyl]-1-(1-methyl-1H-pyrazol-4-yl)pyridazin-4(1H)-one

### Step 1. 3-[3-(5-Hydroxypyrimidin-2-yl)benzyl]-1-(1-methyl-1H-pyrazol-4-yl)pyridazin-4(1H)-one

To Pd/C (10 wt %; 12 mg) under a nitrogen atmosphere was added a solution of 3-{3-[5-(benzyloxy)pyrimidin-2-yl]benzyl}-1-(1-methyl-1H-pyrazol-4-yl)pyridazin-4(1H)-one (Example #67, 253 mg, 0.56 mmol) in EtOH (4 mL) and DCM (2 mL). The reaction-mixture was stirred overnight under H₂ (1 atm). Subsequently, additional Pd/C (10 wt %) was added and the reaction was stirred at room temperature for an additional 5 hours under H₂ (1 atm). Upon completion, the reaction mixture was filtered through Celite and the solvents were removed in vacuo to provide 3-[3-(5-hydroxypyrimidin-2-yl)benzyl]-1-(1-methyl-1H-pyrazol-4-yl)pyridazin-4(1H)-one.

LRMS (APCI) calc'd for C19H17N6O2 [M+H]⁺: 361. Found: 361.

The following intermediates were prepared from Example #71 and Intermediates #106-107 according to Scheme 4 following a similar procedure described for Example #187, which can be achieved by those of ordinary skill in the art of organ c synthesis.

| | | | Exact |
|---|---|---|---|
| | | | Mass |
| Intermediate | Structure | IUPAC Name | [M + H]+ |
| 139 | 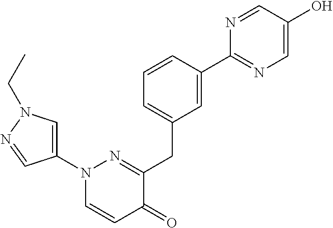 | 1-(1-ethyl-1H-pyrazol-4- yl)-3-[3-(5- hydroxypyrimidin-2- yl)benzyl]pyridazin-4(1H)- one | Calc'd 375, found 375 |
| 140 | 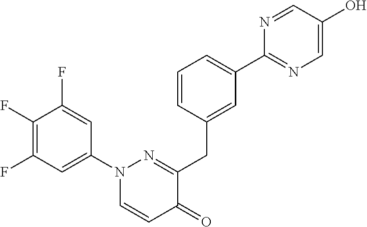 | 3-[3-(5-hydroxypyrimidin- 2-yl)benzyl]-1-(3,4,5- trifluorophenyl)pridazin- 4(1H)-one | Calc'd 411, found 411 |
| 141 | 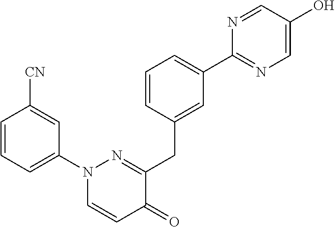 | 3-{3-[3-(5- hydroxypyrimidin-2- yl)benzyl]-4-oxopyridazin- 1(4H)-yl}benzonitrile | Calc'd 382, found 382 |

### Scheme 4

### Example #188

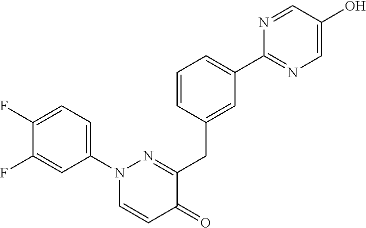

### 1-(3,4-Difluorophenyl)-3-[3-(5-hydroxypyrimidin-2-yl)benzyl]pyridazin-4(1H)-one

### Step 1. 1-(3,4-Difluorophenyl)-3-[3-(5-hydroxypyrimidin-2-yl)benzyl]pyridazin-4(1H)-one

To a solution of 3-{3-[5-(benzyloxy)pyrimidin-2-yl]benzyl}-1-(3,4-difluorophenyl)pyridazin-4(1H)-one (Example #124, 1.05 g, 2.1 mmol) in DCM (21 mL) was added boron tribromide (1M in DCM, 3.0 mL, 3.0 mmol) and the reaction was stirred for 1 hour. Upon completion MeOH was added, the solvent was evaporated in vacuo and the residue was purified by flash chromatography (MPLC, 0-20% MeOH-DCM) to give 1-(3,4-difluorophenyl)-3-[3-(5-hydroxypyrimidin-2-yl)benzyl]pyridazin-4(1H)-one.

LRMS (ESI) calc'd for C21H15F2N4O2 [M+H]⁺: 393. Found: 393.

The following example was prepared from Example #125 according to Scheme 4 following a similar procedure described for Example #188, which can be achieved by those of ordinary skill in the art of organic synthesis.

| | | | Exact |
|---|---|---|---|
| | | | Mass |
| Example | Structure | IUPAC Name | [M + H]+ |
| 189 | 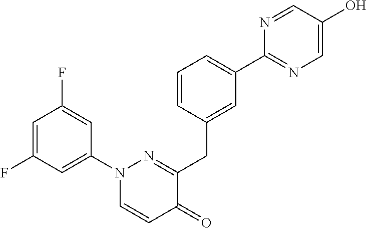 | 1-(3,5-difluorophenyl)-3- [3-(5-hydroxypyrimidin-2- yl)benzyl]pyridazin-4(1H)- one | Calc'd 393, found 393 |

The following intermediates were prepared from Intermediates #108-109 according to Scheme 4 following a similar procedure described for Example #188, which can be achieved by those of ordinary skill in the art of organic synthesis.

| | | | Exact |
|---|---|---|---|
| | | | Mass |
| Intermediate | Structure | IUPAC Name | [M + H]+ |
| 142 | 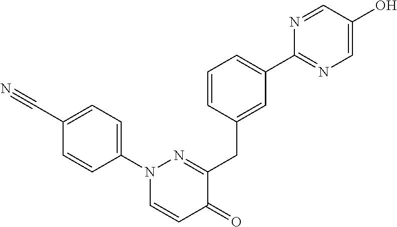 | 4-[3-[3-(5- hydroxypyrimidin-2- yl)benzyl]-4- oxopyridazin-1(4H)- yl]benzonitrile | Calc'd 382, found 382 |
| 143 | 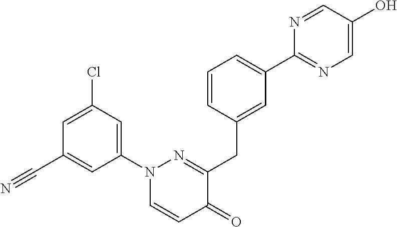 | 3-chloro-5-[3-[3-(5- hydroxypyrimidin-2- yl)benzyl]-4- oxopyridazin-1(4H)- yl]benzonitrile | Calc'd 416, found 416 |

### Scheme 4

### Example #190

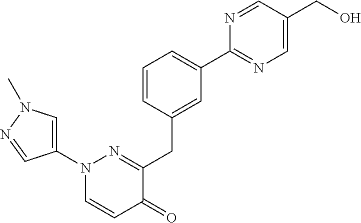

### 3-{3-[5-(Hydroxymethyl)pyrimidin-2-yl]benzyl}-1-(1-methyl-1H-pyrazol-4-yl)pyridazin-4(1H)-one

### Step 1. 3-{3-[5-(Hydroxymethyl)pyrimidin-2-yl]benzyl}-1-(1-methyl-1-pyrazol-4-yl)pyridazin-4(1H)-one

To a 5 mL microwave vial equipped with a stir bar was added. 3-{3-[5-({[tert-butyl(dimethyl)silyl]oxy}methyl)pyrimidin-2-yl]benzyl}-1-(1-methyl-1H-pyrazol-4-yl)pyridazin-4(1H)-one (Intermediate #117, 214 mg, 0.44 mmol) and TBAF (1.0 M in THF, 0.9 mL, 0.9 mmol). The reaction was stirred at room temperature for 1.5 hours. Saturated NaHCO₃ was added and the products extracted into EtOAc. The combined organics were concentrated in vacuo. The residue was purified by flash chromatography (MPLC, 0-15% MeOH-DCM) to give 3-{3-[5-(hydroxymethyl)pyrimidin-2-yl]benzyl}-1-(1-methyl-1H-pyrazol-4-yl)pyridazin-4(1H)-one.

LRMS (ESI) calc'd for C20H19N6O2 [M+H]⁺: 375. Found: 375.

### Scheme 4

### Example #191

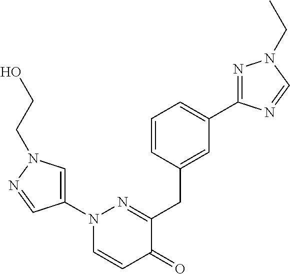

### 3-[3-(1-Ethyl-1H-1,2,4-triazol-3-yl)benzyl]-1-[1-(2-hydroxyethyl)-1H-pyrazol-4-yl]pyridazin-4(1H)-one

### Step 1. 3-[3-(1-Ethyl-1H-1,2,4-triazol-3-yl)benzyl]-1-[1-(2-hydroxyethyl)-1H-pyrazol-4-yl]pyridazin-4(1H)-one

1-{1-[2-(Benzyloxy)ethyl]-1H-pyrazol-4-yl}-3-[3-(1-ethyl-1H-1,2,4-triazol-3-yl)benzyl]pyridazin-4(1H)-one (Example #88, 95 mg, 0.198 mmol) was taken up in DCM (5 mL) and cooled to 0° C. while stirring under N₂ gas. BBr₃ (1 M in DCM, 0.99 mL, 0.99 mmol) was added drop-wise to the reaction flask. The mixture was removed from the ice bath and 15, allowed to stir for 2 hours, eventually reaching ambient temperature. Water was added dropwise followed by saturated Na₂CO₃ (10 mL) to quench and the products were extracted into MeOH/DCM (1:10 mixture, 3×). The combined organic extracts were washed with brine, dried over Na₂SO₄, filtered and concentrated in vacuo. Purification of the residue by flash chromatography (MPLC, 0-20%, MeOH-EtOAc) gave 3-[3-(1-ethyl-1H-1,2,4-triazol-3-yl)benzyl]-1-[1-(2-hydroxyethyl)-1H-pyrazol-4-yl]pyridazin-4(1H)-one.

LRMS (ESI) calc'd for C20H22N7O2 [M+H]⁺: 392. found 392.

The following example was prepared from Example #89 according to Scheme 4 following a similar procedure described for Example #191, which can be achieved by those of ordinary skill in the art of organic chemistry.

| | | | Exact |
|---|---|---|---|
| | | | Mass |
| Example | Structure | IUPAC Name | [M + H]+ |
| 192 | 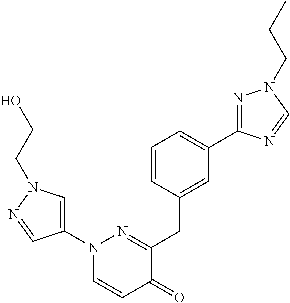 | 1-[1-(2- hydroxyethyl)- 1H-pyrazol-4-yl]- 3-[3-(1-propyl- 1H-1,2,4-triazol-3- yl)benzyl]pyridazin- 4(1H)-one | Calc'd 406, found 406 |

### Scheme 4

### Example #193

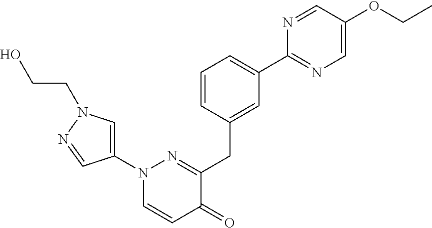

### 3-[3-(5-ethoxypyrimidin-2-yl)benzyl]-1-[1-(2-hydroxyethyl)-1H-pyrazol-4-yl]pyridazin-4(1H)-one

### Step 1. 3-[3-(5-Ethoxypyrimidin-2-yl)benzyl]-1-[1-(2-hydroxyethyl)-1H-pyrazol-4-yl]pyridazin-4(1H)-one

1-{1-[2-(benzyloxy)ethyl]-1H-pyrazol-4-yl}-3-[3-(5-ethoxypyrimidin-2-yl)benzyl]pyridazin-4(1H)-one (Intermediate #111, 42 mg, 0.083 mmol), ammonium formate (41.7 mg, 0.661 mmol) and Pd/C (10 wt %, 105 mg, 0.099 mmol) were taken up in acetone (4.2 mL) in a 5 mL microwave vial. The reaction was stirred at 60° C. for 18 hours. Additional ammonium formate (41.7 mg, 0.661 mmol) was added and stirring at 60° C. continued for 24 hours. The catalyst was removed by filtering through Celite and the filtrate concentrated in vacuo. Purification of the residue by reverse phase preparative HPLC (20-80% MeCN—H₂O, 0.1% TFA) gave 3-[3-(5-ethoxypyrimidin-2-yl)benzyl]-1-[1-(2-hydroxyethyl)-1H-pyrazol-4-yl]pyridazin-4(1H)-one as a pale yellow solid.

LRMS (ESI) calc'd for C22H23N6O3 [M+H]⁺: 419. found 419.

### Scheme 4

### Example #194

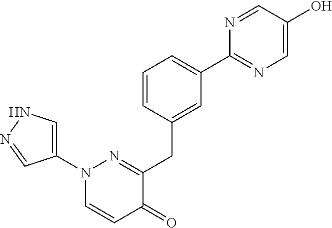

### 3-[3-(5-Hydroxypyrimidin-2-yl)benzyl]-1-(1H-pyrazol-4-yl)pyridazin-4(1H)-one

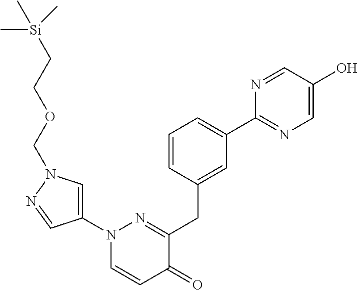

### Step 1. 3-[3-(5-Hydroxypyrimidin-2-yl)benzyl]-1-(1-{[2-(trimethylsilyl)ethoxy]methyl}-1H-pyrazol-4-yl)pyridazin-4(1H)-one

To a microwave vial was added 3-{3-[5-(benzyloxy)pyrimidin-2-yl]benzyl}-(1-{[2-(trimethylsilyl)ethoxy]methyl}-1H-pyrazol-4-yl)pyridazin-4(1H)-one (Intermediate #112, 23 mg, 0.041 mmol) and Pd/C (10 wt %, 13 mg, 0.012 mmol). The vial was sealed under N₂ and EtOH (370 L) and DCM (370 μL) were syringed in. The vial was stirred under a balloon of H₂ for 18 hours. The crude reaction mixture was diluted with 10 mL of a 1:1 solution of DCM:EtOH. The crude was filtered through a column pre-packed with Celite. The resulting organics were concentrated in vacuo and the material was carried on without further purification.

LRMS (ESI) calc'd for C24H29N6O3Si [M+H]⁺: 477. Found: 477.

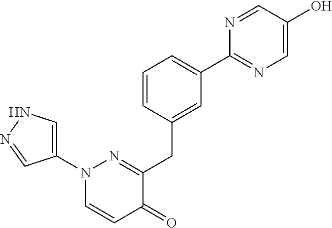

### Step 2. 3-[3-(5-Hydroxypyrimidin-2-yl)benzyl]-1-(1H-pyrazol-4-yl)pyridazin-4(1H)-one

To a 2 mL microwave-vial equipped with a stir bar was added 3-[3-(5-hydroxypyrimidin-2-yl)benzyl]-1-(1-{[2-(trimethylsilyl)ethoxy]methyl}-1H-pyrazol-4-yl)pyridazin-4(1H)-one 20 mg, 0.042 mmol) and 1 N HCl (420 μL, 0.420 mmol). The vial was sealed and the reaction mixture was heated to 50° C. for one hour. The crude reaction mixture was diluted with ethyl acetate and concentrated in vacuo. The residue was purified by flash chromatography (MPLC, 0-15% MeOH-DCM) to give 3-[3-(5-hydroxypyrimidin-2-yl)benzyl]-1-(1H-pyrazol-4-yl)pyridazin-4(1H)-one.

LRMS (ESI) calc'd for C18H15N6O2 [M+H]⁺: 347. Found: 347.

The following examples were prepared from Intermediates #110, 113-114 according to Scheme 4 following a similar procedure described for Example #194 Step 2, which can be achieved by those of ordinary skill in the art of organic synthesis.

| | | | Exact |
|---|---|---|---|
| | | UPAC | Mass |
| Example | Structure | Name | [M + H]⁺ |
| 195 | 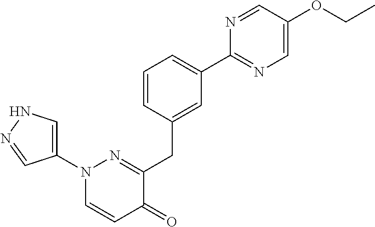 | 3-[3-(5- ethoxyprimidin-2- yl)benzyl]-1- (1H-pyrazol-4- yl)pyridazin- 4(1H)-one | Calc'd 375, found 375 |
| 196 | 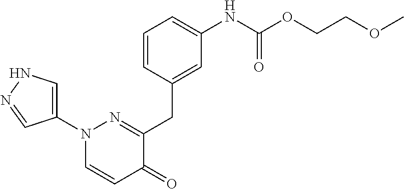 | 2-methoxyethyl (3-{[4-oxo-1- (1H-pyrazol- 4-yl)-1,4- dihydropyridazin-3- yl]methyl}phenyl) carbamate | Calc'd 370, found 370 |
| 197 | 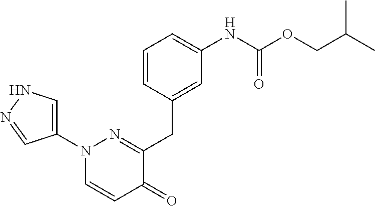 | isobutyl (3- {[4-oxo-1- (1H-pyrazol- 4-yl)-1,4- dihydropyridazin-3- yl]methyl}phenyl) carbamate | Calc'd 368, found 368 |

### Scheme 4

### Example #198

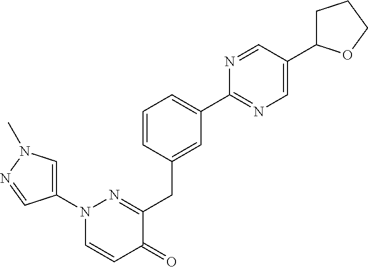

### Step 1. rac-1-(1-Methyl-1H-pyrazol-4-yl)-3-{3-[5-(tetrahydrofuran-2-yl)pyrimidin-2-yl]benzyl}pyridazin-4(1H)-one

rac-3-{3-[5-(2,5-Dihydrofuran-2-yl)pyrimidin-2-yl]benzyl}-1-(1-methyl-1H-pyrazol-4-yl)pyridazin-4(1H)-one (Example #174, 97 mg, 0.24 mmol) and 5% Pd/C (Type A470129-5 59.15% water; Johnson Matthey, 10 mg) were added to a vial, and placed under nitrogen. Ethanol (1 mL) was added, followed by ethyl acetate (1 mL). The resulting suspension was stirred under a hydrogen atmosphere (balloon pressure) at ambient temperature overnight. The reaction mixture was diluted with EtOAc and filtered through Celite, rinsing with EtOAc. Silica gel was added to the filtrate and the resulting mixture was concentrated to dryness. The resulting solid was purified by flash chromatography (MPLC, 10% MeOH-EtOAc) to afford rac-1-(1-methyl-1H-pyrazol-4-yl)-3-{3-[5-(tetrahydrofuran-2-yl)pyrimidin-2-yl]benzyl}pyridazin-4(1H)-one as a white foam.

LRMS (ESI) calc'd for C23H23N6O2 [M+H]⁺, 415. found 415.

The following example was prepared from Example #175 according to Scheme 4 following a similar procedure described for Example #198, which can be achieved by those of ordinary skill in the art of organic synthesis.

| | | | Exact |
|---|---|---|---|
| | | IUPAC | Mass |
| Example | Structure | Name | [M + H]⁺ |
| 199 | 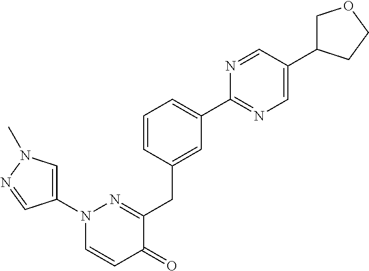 | rac-1-(1- methyl-1H- pyrazol-4-yl)- 3-{3-[5- (tetrahydrofuran- 3-yl)pyrimidin-2- yl]benzyl} pyridazin-4(1H)- one | Calc'd 415, found 415 |

### Scheme 4

### Example #200

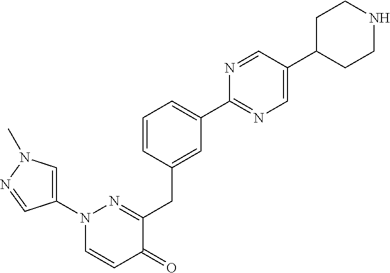

### 1-(1-Methyl-1H-pyrazol-4-yl)-3-{3-[5-(piperidin-4-yl)pyrimidin-2-yl]benzyl}pyridazin-4(1H)-one

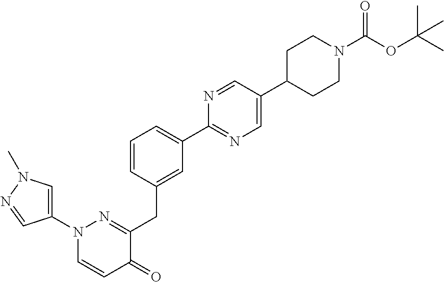

### Step 1. tert-Butyl 4-[2-(3-{[1-1-methyl-1H-pyrazol-4-yl)-4-oxo-1,4-dihydropyridazin-3-yl]methyl}phenyl)pyrimidin-5-yl]piperidine-1-carboxylate

ter-t-Butyl 4-[2-(3-{[1-(4-methyl-1H-pyrazol-4-yl)-4-oxo-1,4-dihydropyridazin-3-yl]methyl}phenyl)pyrimidin-5-yl]-3,6-dihydropyridine-1(2H)-carboxylate (Intermediate #118, 50 mg, 0.095 mmol) and 5% Pd/C (Type A470129-5 59.15% water; Johnson-Matthey) (5 mg) were added to a vial, then placed under nitrogen. Ethanol (1 mL) was added, followed by MeOH (1 mL). The resulting suspension was stirred at ambient temperature under hydrogen (balloon pressure) for 15 h. In order to drive the reaction to completion, additional MeOH (1 mL) was added and the reaction mixture was heated to 5.0° C. for 3 hours. The reaction mixture was cooled, diluted with MeOH and filtered through Celite. The filtrate was concentrated to afford an oil that was taken up in DCM and hexanes. Concentration afforded tert-butyl 4-[2-(3-{[1-(1-methyl-1H-pyrazol-4-yl)-4-oxo-1,4-dihydropyridazin-3-yl]methyl}phenyl)pyrimidin-5-yl]piperidine-1-carboxylate as an off-white solid.

LRMS (ESI) calc'd for C29H34N7O3 [M+H]⁺, 528. found 528.

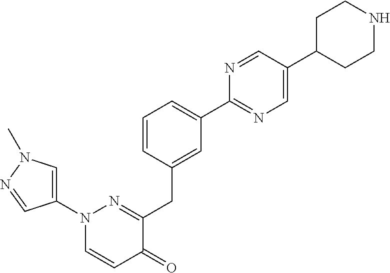

### Step 2. 1-(1-methyl-1H-pyrazol-4-yl)-3-{3-[5-(piperidin-4-yl)pyrimidin-2-yl]benzyl}pyridazin-4(1H)-one

To a stirred solution of tert-butyl 4-[2-(3-{[1-(1-methyl-1H-pyrazol-4-yl)-4-oxo-1,4-dihydropyridazin-3-yl]methyl}phenyl)pyrimidin-5-yl]piperidine-1-carboxylate (50 mg, 0.095 mmol) in MeOH (1 mL) was added 4N HCl in dioxane (1 mL). The resulting suspension was stirred at room temperature for 30 min. The reaction mixture was diluted with MeOH and concentrated to afford a residue that was dissolved in MeCN/water, and filtered through a PL-HCO₃ cartridge (Stratospheres™, 0.9-mmol). The filtrate was lyophilized to afford 1-(1-methyl-1H-pyrazol-4-yl)-3-{3-[5-(piperidin-4-yl)pyrimidin-2-yl]benzyl}pyridazin-4(1H)-one as a tan solid.

LRMS (ESI) calc'd for C24H26N7O [M+H]⁺, 428.2. found 428.

### Scheme 5

### Examples #201 and 202

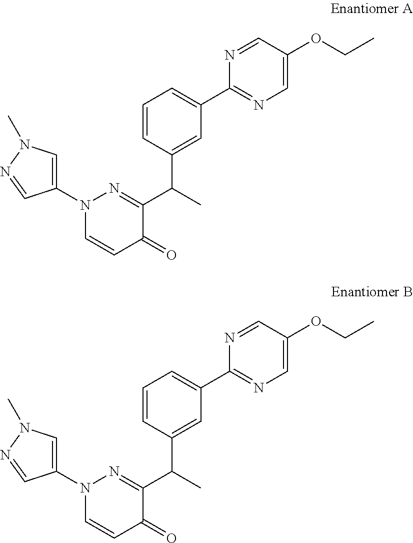

### 3-{(1S or R)-1-[3-(5-Ethoxypyrimidin-2-yl)phenyl]ethyl}-1-(1-methyl-1H-pyrazol-4-yl)pyridazin-4(1H)-one (Enantiomer A) and 3-{(1R or S)-1-[3-(5-ethoxypyrimidin-2-yl)phenyl]ethyl}-1-(1-methyl-1H-pyrazol-4-yl)pyridazin-4(1H)-one (Enantiomer B)

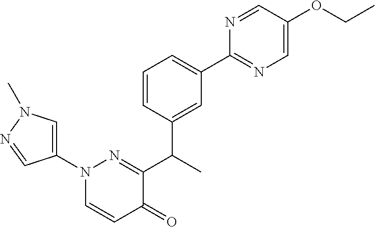

### Step 1. rac-3-{1-[3-(5-Ethoxypyrimidin-2-yl)phenyl]ethyl}-1-(1-methyl-1H-pyrazol-4-yl)pyridazin-4(1H)-one

3-[3-(5-Ethoxypyrimidin-2-yl)benzyl]-1-(1-methyl-1H-pyrazol-4-yl)pyridazin-4(1H)-one (Example #3, 150 mg, 0.37 mmol) was dissolved in DMF (2 mL) and iodomethane (0.07 mL, 1.16 mmol) was added. The reaction mixture was cooled to 00° C. and NaH (60 wt %, 28 mg, 1.16 mmol) was added. The reaction mixture was stirred at 0° C. gradually warming to r.t. for 6 hrs. The reaction mixture was diluted with EtOAc, washed with saturated NaHCO₃ and the aqueous phase was extracted with further portions of EtOAc. The combined organic extracts were dried over Na₂SO₄, filtered and concentrated in vacuo while loading onto silica. The crude residue was purified by flash chromatography (MPLC, 0-10% EtOAc-MeOH) to give rac-3-{1-[3-(5-ethoxypyrimidin-2-yl)phenyl]ethyl}-1-(1-methyl-1H-pyrazol-4-yl)pyridazin-4(1H)-one as a white foam.

LRMS (ESI) calc'd for C22H23N6O2 [M+H]⁺: 403. Found: 403.

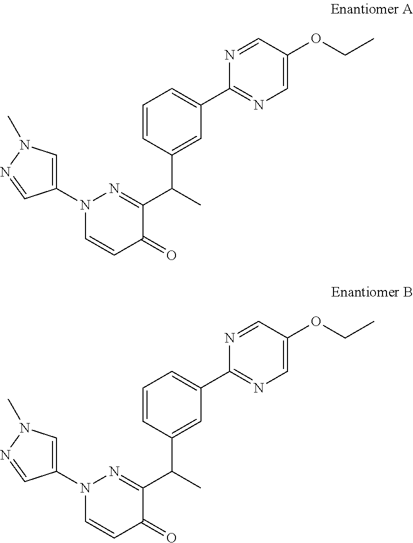

### Step 2. 3-{(1S or R)-1-[3-(5-Ethoxypyrimidin-2-yl)phenyl]ethyl}-1-(1-methyl-1H-pyrazol-4-yl)pyridazin-4(1H)-one (Enantiomer A) and 3-{(1R or S)-1-[3-(5-ethoxypyrimidin-2-yl)phenyl]ethyl}-1-(1-methyl-1H-pyrazol-4-yl)pyridazin-4(1H)-one (Enantiomer B)

rac-3-{1-[3-(5-Ethoxypyrimidin-2-yl)phenyl]ethyl}-1-(1-methyl-1H-pyrazol-4-yl)pyridazin-4(1H)-one (85 mg, 0.21 mmol) was resolved by SFC (Berger Multigram II SFC, column: Chiral Technology AS-H 2.1×25 cm, 5 μM, mobile phase: 35% methanol/65% CO₂₍₁₎, flow rate: 70 mL/min, 6 min run time) to give 3-{(1S or R)-1-[3-(5-ethoxypyrimidin-2-yl)phenyl]ethyl}-1-(1-methyl-1H-pyrazol-4-yl)pyridazin-4(1H)-one (Enantiomer A, Example #201) as a white foam and 3-{(1R or S)-1-[3-(5-ethoxypyrimidin-2-yl)phenyl]ethyl}-1-(1-methyl-1H-pyrazol-4-yl)pyridazin-4(1H)-one (Enantiomer B, Example #202) as a white foam.

### Example #201

LRMS (ESI)-calc'd for C22H23N6O2 [M+H]⁺: 403. Found: 403.

### Example #202

LRMS (ESI) calc'd for C22H23N6O2 [M+H]⁺: 403. Found: 403.

### Scheme 5

### Examples #203 and 204

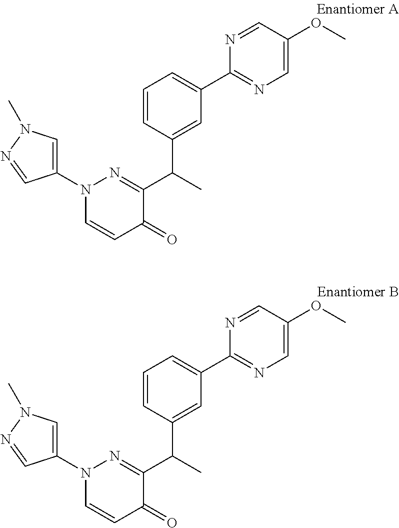

### 3-{(1S or R)-1-[3-(5-Methoxypyrimidin-2-yl)phenyl]ethyl}-1-(1-methyl-1H-pyrazol-4-yl)pyridazin-4(1H)-one (Enantiomer A) and 3-{(1R or S)-1-[3-(5-methoxypyrimidin-2-yl)phenyl]ethyl}-1-(1-methyl-1H-pyrazol-4-yl)pyridazin-4(1H)-one (Enantiomer B)

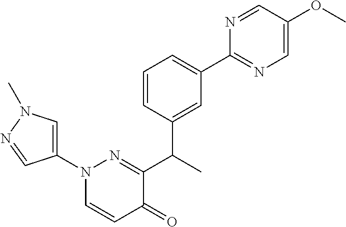

### Step 1. rac-3-{1-[3-(5-Methoxypyrimidin-2-yl)phenyl]ethyl}-1-(1-methyl-1H-pyrazol-4-yl)pyridazin-4(1H)-one

3-[3-(5-Methoxypyrimidin-2-yl)benzyl]-1-(1-methyl-1H-pyrazol-4-yl)pyridazin-4(1H)-one (Example #5, 416 mg, 1.11 mmol) and iodomethane (0.208 mL, 3.33 mmol) were taken up in DMF (5.5 mL)/THF (5.5 mL). Sodium hydride (60 wt %, 133 mg, 3.33 mmol) was added and the resulting mixture stirred at room temperature for 3 hours. Saturated NH₄Cl was added and the products extracted into EtOAc (X2). The combined organic extracts were washed with brine, dried over Na₂SO₄, filtered and concentrated in vacuo. Purification of the residue by flash chromatography (MPLC, 0-15% MeOH-EtOAc) gave rac-3-{1-[3-(5-methoxypyrimidin-2-yl)phenyl]ethyl}-1-(1-methyl-1H-pyrazol-4-yl)pyridazin-4(1H)-one as an off-white solid.

LRMS (ESI) calc'd for C21H21N6O2 [M+H]⁺: 389. Found: 389.

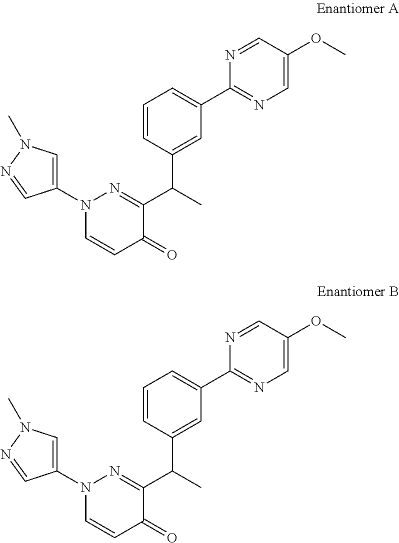

### Step 2. 3-{(1S or R)-1-[3-(5-Methoxypyrimidin-2-yl)phenyl]ethyl}-1-(1-methyl-1H-pyrazol-4-yl)pyridazin-4(1H)-one (Enantiomer A) and 3-{(1R or S)-1-[3-(5-methoxypyrimidin-2-yl)phenyl]ethyl}-1-(1-methyl-1H-pyrazol-4-yl)pyridazin-4(1H)-one (Enantiomer B)

rac-3-{1-[3-(5-Methoxypyrimidin-2-yl)phenyl]ethyl}-1-(1-methyl-1H-pyrazol-4-yl)pyridazin-4(1H)-one (378 mg, 0.97 mmol) was resolved by SFC (Berger Multigram II SFC, column: Chiral Technology AS-H 2.1×25 cm, 5 uM, mobile phase: 40% methanol/60% CO_{2(I)}, flow rate: 60 mL/min, 8 min run time) to give 3-((1S or R)-1-[3-(5-methoxypyrimidin-2-yl)phenyl]ethyl)-1-(1-methyl-1H-pyrazol-4-yl)pyridazin-4(1H)-one (Enantiomer A, Example #203) and 3-{(1R or S)-1-[3-(5-methoxypyrimidin-2-yl)phenyl]ethyl}-1-(1-methyl-1H-pyrazol-4-yl)pyridazin-4(1H)-one (Enantiomer B, Example #204).

### Example #203

LRMS (ESI) calc'd for C21H21N6O2 [M+H]⁺: 389. Found: 389.

### Example #204

LRMS (ESI) calc'd for C21H21N6O2 [M+H]⁺: 389. Found: 389.

The following examples were prepared from Examples #3-5, 71-72, 150-151, 154, 156-157, 160-165, 277, 386, 391, 407, 465 and 472-473 according to Scheme 5 following similar procedures described for Examples #201-204, which can be achieved by those of ordinary skill in the art of organic synthesis.

| | | | Exact |
|---|---|---|---|
| | | | Mass |
| Example | Structure | IUPAC Name | [M + H]+ |
| 205 | 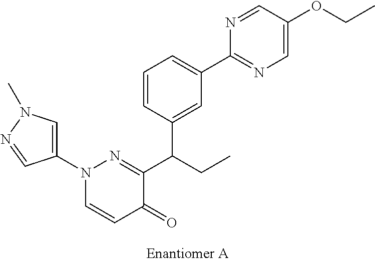 | 3-{(1S or R)-1-[3-(5- ethoxypyrimidin-2- yl)phenyl]propyl}- 1-(1-methyl-1H- pyrazol-4- yl)pyridazin- 4(1H)-one | Calc'd 417, found 417 |
| 206 | 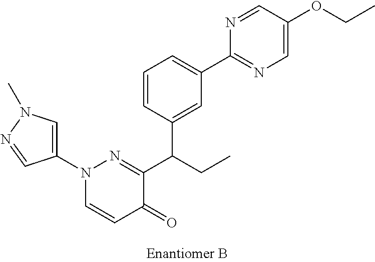 | 3-{(1R or S)-1-[3-(5- ethoxypyrimidin-2- yl)phenyl]propyl}- 1-(1-methyl-1H- pyrazol-4- yl)pyridazin- 4(1H)-one | Calc'd 417, found 417 |
| 207 | 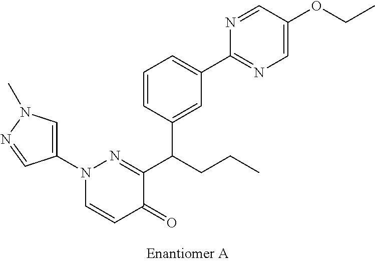 | 3-{(1S or R)-1-[3-(5- ethoxypyrimidin-2- yl)phenyl]butyl}- 1-(1-methyl-1H- pyrazol-4- yl)pyridazin- 4(1H)-one | Calc'd 431, found 431 |
| 208 | 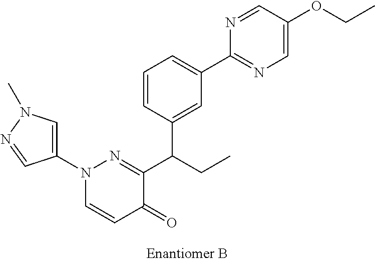 | 3-{(1R or S)-1-[3-(5- ethoxypyrimidin-2- yl)phenyl]butyl}- 1-(1-methyl-1H- pyrazol-4- yl)pyridazin- 4(1H)-one | Calc'd 431, found 431 |
| 209 | 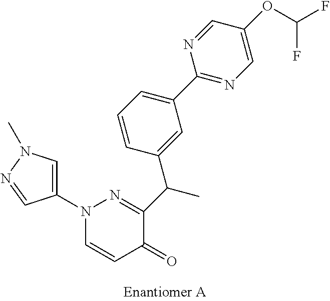 | 3-[(1S or R)-1-{3-[5- (difluoromethoxy) pyrimidin-2- yl]phenyl}ethyl]- 1-(1-methyl-1H- pyrazol-4- yl)pyridazin- 4(1H)-one | Calc'd 425, found 425 |
| 210 | 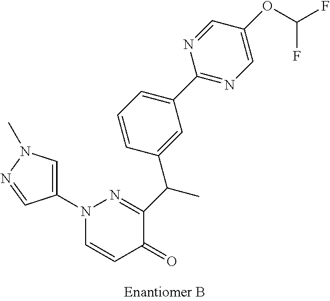 | 3-[(1R or S)-1-{3-[5- (difluoromethoxy) pyrimidin-2- yl]phenyl}ethyl]- 1-(1-methyl-1H- pyrazol-4- yl)pyridazin- 4(1H)-one | Calc'd 425, found 425 |
| 211 | 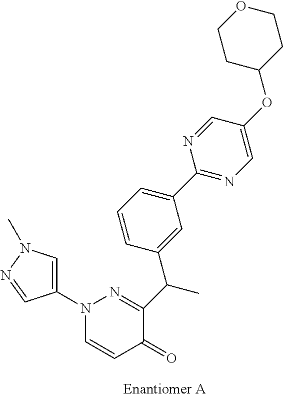 | 1-(1-methyl-1H- pyrazol-4-yl)-3- [(1S or R)-1-{3- [5-(tetrahydro- 2H-pyran-4- yloxy)pyrimidin-2- yl]phenyl}ethyl] pyridazin-4(1H)- one | Calc'd 459, found 459 |
| 212 | 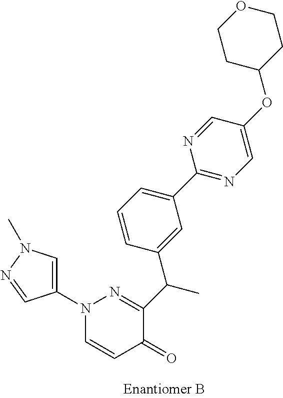 | 1-(1-methyl-1H- pyrazol-4-yl)-3- [(1R or S)-1-{3- [5-(tetrahydro- 2H-pyran-4- yloxy)pyrimidin-2- yl]phenyl}ethyl] pyridazin-4(1H)- one | Calc'd 459, found 459 |
| 213 | 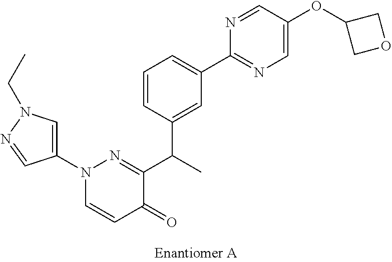 | 1-(1-ethyl-1H- pyrazol-4-yl)-3- ((1S or R)-1-{3- [5-(oxetan-3- yloxy)pyrimidin-2- yl]phenyl}ethyl) pyridazin-4(1H)- one | Calc'd 445, found 455 |
| 214 | 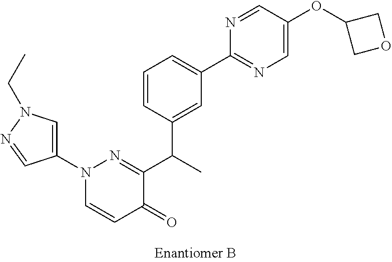 | 1-(1-ethyl-1H- pyrazol-4-yl)-3- ((1R or S)-1-{3- [5-(oxetan-3- yloxy)pyrimidin-2- yl]phenyl}ethyl) pyridazin-4(1H)- one | Calc'd 445, found 445 |
| 215 | 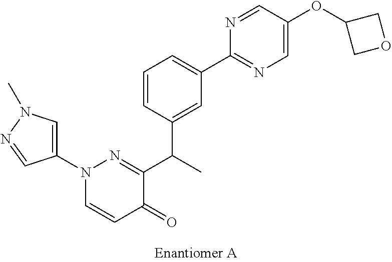 | 1-(1-methyl-1H- pyrazol-4-yl)-3- ((1S or R)-1-{3- [5-(oxetan-3- yloxy)pyrimidin-2- yl]phenyl}ethyl) pyridazin-4(1H)- one | Calc'd 431, found 431 |
| 216 | 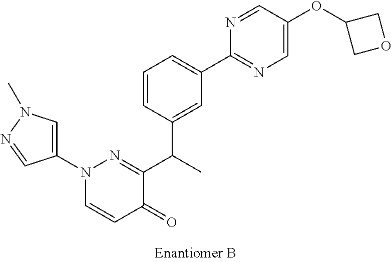 | 1-(1-methyl-1H- pyrazol-4-yl)-3- ((1R or S)-1-{3- [5-(oxetan-3- yloxy)pyrimidin-2- yl]phenyl}ethyl) pyridazin-4(1H)- one | Calc'd 431, found 431 |
| 217 | 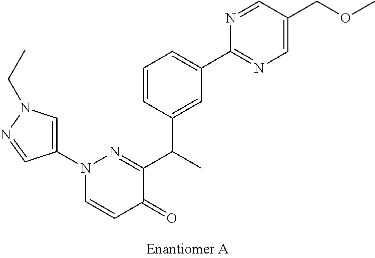 | 1-(1-ethyl-1H- pyrazol-4-yl)-3- ((1S or R)-1-{3-[5- (methoxymethyl) pyrimidin-2- yl]phenyl}ethyl) pyridazin-4(1H)- one | Calc'd 417, found 417 |
| 218 | 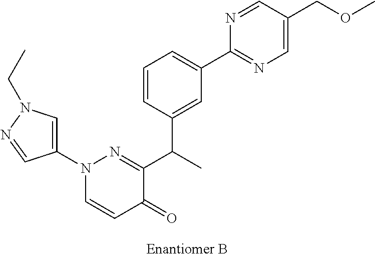 | 1-(1-ethyl-1H- pyrazol-4-yl)-3- ((1R or S)-1-{3-[5- (methoxymethyl) pyrimidin-2- yl]phenyl}ethyl) pyridazin-4(1H)- one | Calc'd 417, found 417 |
| 219 | 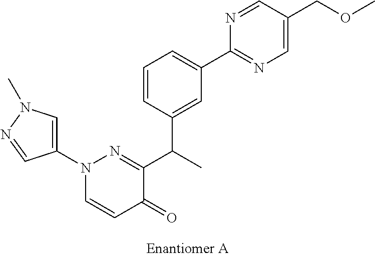 | 3-((1S or R)-1-{3-[5- (methoxymethyl) pyrimidin-2- yl]phenyl}ethyl)- 1-(1-methyl-1H- pyrazol-4- yl)pyridazin- 4(1H)-one | Calc'd 403, found 403 |
| 220 | 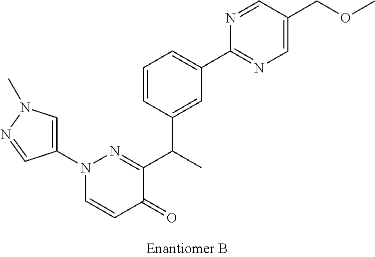 | 3-((1R or S)-1-{3-[5- (methoxymethyl) pyrimidin-2- yl]phenyl}ethyl)- 1-(1-methyl-1H- pyrazol-4- yl)pyridazin- 4(1H)-one | Calc'd 403, found 403 |
| 221 | 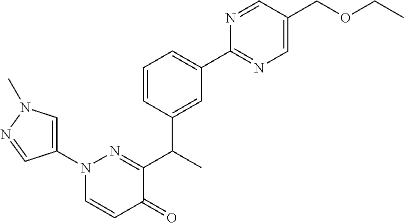 | rac-3-(1-{3-[5- (ethoxymethyl) pyrimidin-2- yl]phenyl}ethyl)- 1-(1-methyl-1H- pyrazol-4- yl)pyridazin- 4(1H)-one | Calc'd 417, found 417 |
| 222 | 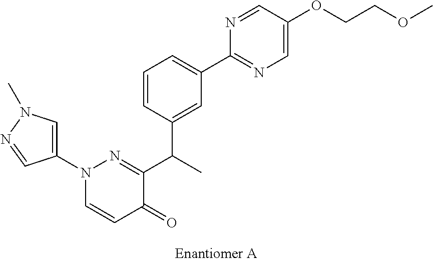 | 3-((1S or R)-1-{3-[5-(2- methoxyethoxy) pyrimidin-2- yl]phenyl}ethyl)- 1-(1-methyl-1H- pyrazol-4- yl)pyridazin- 4(1H)-one | Calc'd 433, found 433 |
| 223 | 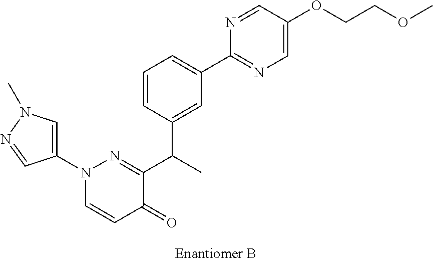 | 3-((1R or S)-1-{3-[5-(2- methoxyethoxy) pyrimidin-2- yl]phenyl}ethyl)- 1-(1-methyl-1H- pyrazol-4- yl)pyridazin- 4(1H)-one | Calc'd 433, found 433 |
| 224 | 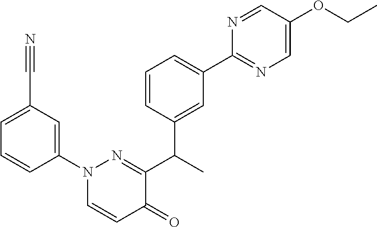 | rac-3-[3-{1-[3-(5- ethoxypyrimidin-2- yl)phenyl]ethyl}- 4-oxopyridazin- 1(4H)- yl]benzonitrile | Calc'd 424, found 424 |
| 225 | 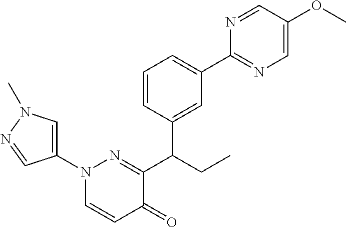 | rac-3-{1-[3-(5- methoxypyrimidin-2- yl)phenyl]propyl}- 1-(1-methyl-1H- pyrazol-4- yl)pyridazin- 4(1H)-one | Calc'd 403, found 403 |
| 226 | 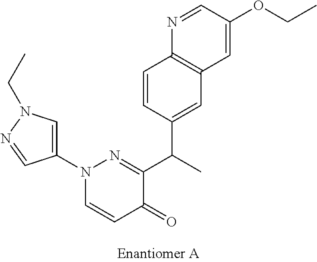 | 3-[(1S or R)-1-(3- ethoxyquinolin-6- yl)ethyl]-1-(1- ethyl-1H-pyrazol- 4-yl)pyridazin- 4(1H)-one | Calc'd 390, found 390 |
| 227 | 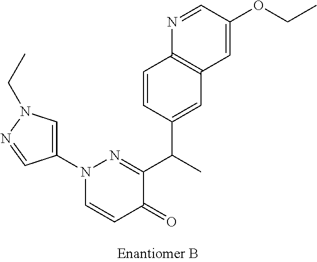 | 3-[(1R or S)-1-(3- ethoxyquinolin-6- yl)ethyl]-1-(1- ethyl-1H-pyrazol- 4-yl)pyridazin- 4(1H)-one | Calc'd 390, found 390 |
| 228 | 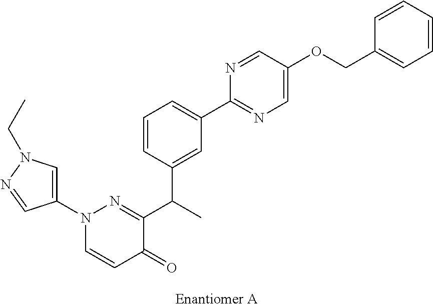 | 3-((1S or R)-1-{3-[5- (benzyloxy) pyrimidin-2- yl]phenyl}ethyl)- 1-(1-ethyl-1H- pyrazol-4- yl)pyridazin- 4(1H)-one | Calc'd 479, found 479 |
| 229 | 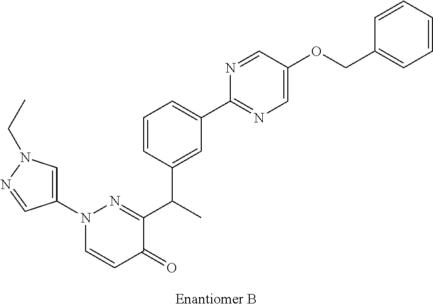 | 3-((1R or S)-1-{3-[5- (benzyloxy)pyrimidin-2- yl]phenyl}ethyl)- 1-(1-ethyl-1H- pyrazol-4- yl)pyridazin- 4(1H)-one | Calc'd 479, found 479 |
| 230 | 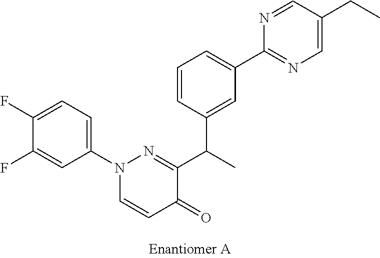 | 1-(3,4- difluorophenyl)- 3-{(1S or R)-1-[3-(5- ethylpyrimidin-2- yl)phenyl]ethyl} pyridazin-4(1H)- one | Calc'd 419, found 419 |
| 231 | 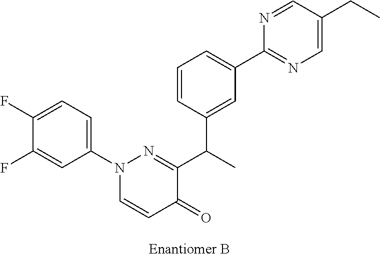 | 1-(3,4- difluorophenyl)- 3-{(1R or S)-1-[3-(5- ethylpyrimidin-2- yl)phenyl]ethyl} pyridazin-4(1H)- one | Calc'd 419, found 419 |
| 232 | 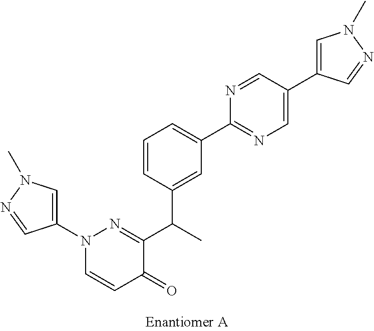 | 1-(1-methyl-1H- pyrazol-4-yl)-3- [(1S or R)-1-{3- [5-(1-methyl-1H- pyrazol-4- yl)pyrimidin-2- yl]phenyl}ethyl] pyridazin-4(1H)- one | Calc'd 439, found 439 |
| 233 | 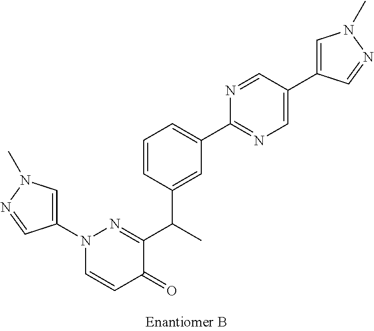 | 1-(1-methyl-1H- pyrazol-4-yl)-3- [(1R or S)-1-{3- [5-(1-methyl-1H- pyrazol-4- yl)pyrimidin-2- yl]phenyl}ethyl] pyridazin-4(1H)- one | Calc'd 439, found 439 |
| 234 | 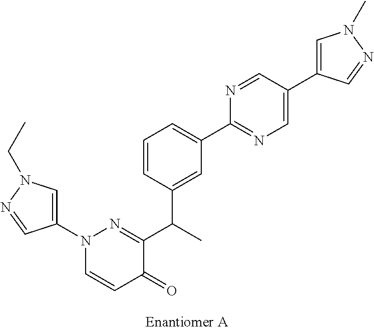 | 1-(1-ethyl-1H- pyrazol-4-yl)-3- [(1S or R)-1-{3- [5-(1-methyl-1H- pyrazol-4- yl)pyrimidin-2- yl]phenyl}ethyl] pyridazin-4(1H)- one | Calc'd 453, found 453 |
| 235 | 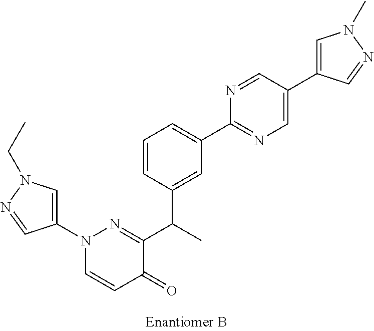 | 1-(1-ethyl-1H- pyrazol-4-yl)-3- [(1R or S)-1-{3- [5-(1-methyl-1H- pyrazol-4- yl)pyrimidin-2- yl]phenyl}ethyl] pyridazin-4(1H)- one | Calc'd 453, found 453 |
| 236 | 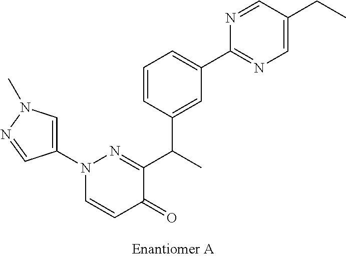 | 3-{(1S or R)-1-[3-(5- ethylpyrimidin-2- yl)phenyl]ethyl}- 1-(1-methyl-1H- pyrazol-4- yl)pyridazin- 4(1H)-one | Calc'd 387, found 387 |
| 237 | 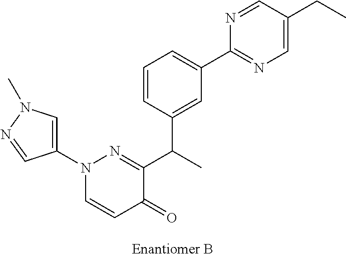 | 3-{(1R or S)-1-[3-(5- ethylpyrimidin-2- yl)phenyl]ethyl}- 1-(1-methyl-1H- pyrazol-4- yl)pyridazin- 4(1H)-one | Calc'd 387, found 387 |
| 238 | 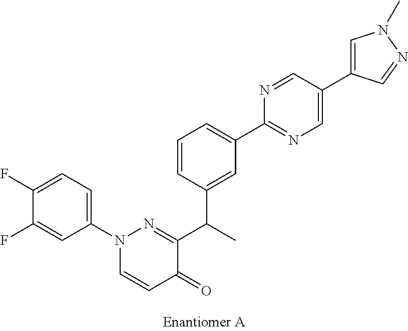 | 1-(3,4- difluorophenyl)- 3-[(1S or R)-1-{3- [5-(1-methyl-1H- pyrazol-4- yl)pyrimidin-2- yl]phenyl}ethyl] pyridazin-4(1H)- one | Calc'd 471, found 471 |
| 239 | 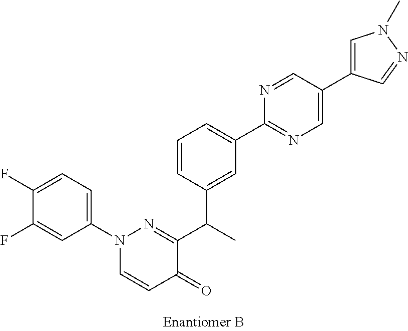 | 1-(3,4- difluorophenyl)- 3-[(1R or S)-1-{3- [5-(1-methyl-1H- pyrazol-4- yl)pyrimidin-2- yl]phenyl}ethyl] pyridazin-4(1H)- one | Calc'd 471, found 471 |
| 240 | 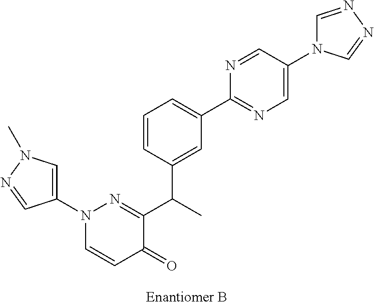 | 1-(1-methyl-1H- pyrazol-4-yl)-3- [(1R or S)-1-{3- [5-(4H-1,2,4- triazol-4- yl)pyrimidin-2- yl]phenyl}ethyl] pyridazin-4(1H)- one | Calc'd 426, found 426 |
| 241 | 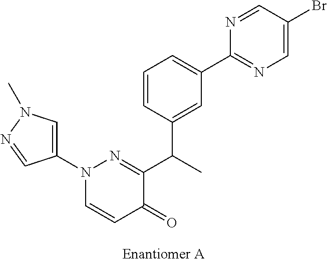 | 3-{(1S or R)-1-[3-(5- bromopyrimidin-2- yl)pheny]ethyl)}- 1-(1-methyl-1H- pyrazol-4- yl)pyridazin- 4(1H)-one | Calc'd 437, found 437 |
| 242 | 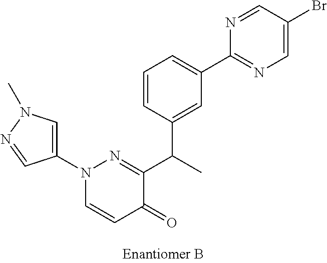 | 3-{(1R or S)-1-[3-(5- bromopyrimidin-2- yl)phenyl]ethyl}- 1-(1-methyl-1H- pyrazol-4- yl)pyridazin- 4(1H)-one | Calc'd 437, found 437 |
| 243 | 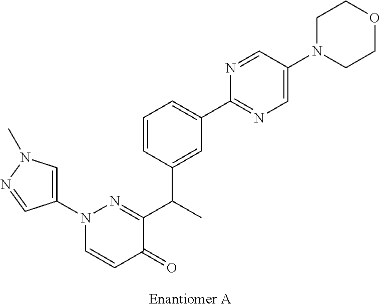 | 1,1-methyl-1H- pyrazol-4-yl)-3- {(1S or R)-1-[3- (5-morpholin-4- ylpyrimidin-2- yl)phenyl]ethyl} pyridazin-4(1H) one | Calc'd 444, found 444 |
| 244 | 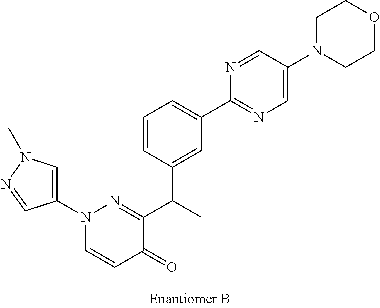 | 1-(1-methyl-1H- pyrazol-4-yl)-3- {(1R or S)-1-[3- (5-morpholin-4- ylpyrimidin-2- yl)phenyl]ethyl} pyridazin-4(1H)- one | Calc'd 444, found 444 |
| 245 | 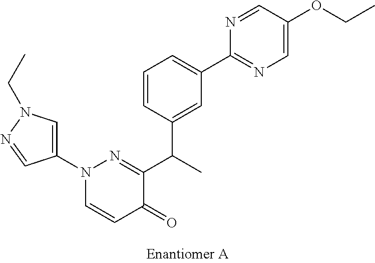 | 3-{(1S or R)-1-[3-(5- ethoxypyrimidin-2- yl)phenyl]ethyl}- 1-(1-ethyl-1H- pyrazol-4- yl)pyridazin- 4(1H)-one | Calc'd 417, found 417 |
| 246 | 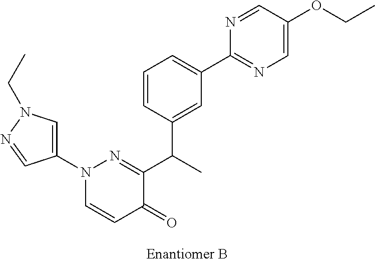 | 3-{(1R or S)-1-[3-(5- ethoxypyrimidin-2- yl)phenyl]ethyl}- 1-(1-ethyl-1H- pyrazol-4- yl)pyridazin- 4(1H)-one | Calc'd 417, found 417 |
| 247 | 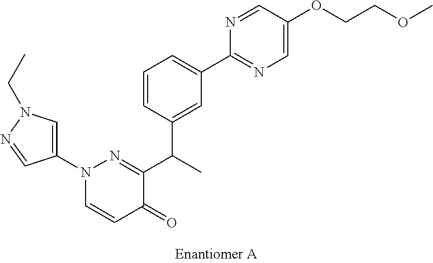 | 1-(1-ethyl-1H- pyrazol-4-yl)-3- ((1S or R)-1-{3-[5-(2- methoxyethoxy) primidin-2- yl]phenyl}ethyl) pyridazin-4(1H)- one | Calc'd 447, found 447 |
| 248 | 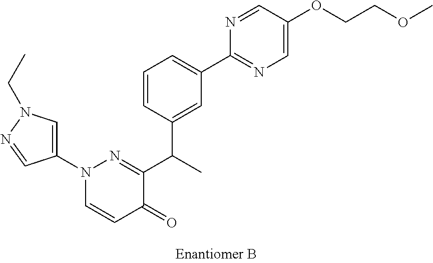 | 1-(1-ethyl-1H- pyrazol-4-yl)-3- ((1R or S)-1-{3-[5-(2- methoxyethoxy) pyrimidin-2- yl]phenyl}ethyl) pyridazin-4(1H)- one | Calc'd 447, found 447 |

The following intermediates were prepared from Intermediates #127-128 and 130 according to Scheme 5 following a similar procedure described for Examples #201-202 Step 1, which can be achieved by those of ordinary skill in the art of organic synthesis.

| | | | Exact |
|---|---|---|---|
| | | | Mass |
| Intermediate | Structure | IUPAC Name | [M + H]+ |
| 144 | 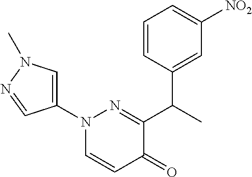 | rac-1-(1-methyl- 1H-pyrazol-4-yl)- 3-[1-(3- nitrophenyl)ethyl-] pyridazin-4(1H)- one | Calc'd 326, found 326 |
| 145 | 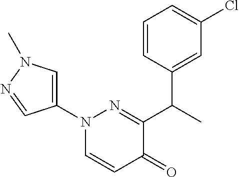 | rac-3-[1-(3- chlorophenyl)ethyl]- 1-(1-methyl- 1H-pyrazol-4- yl)pyridazin- 4(1H)-one | Calc'd 315, found 315 |
| 146 | 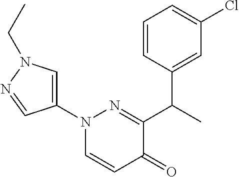 | rac-3-[1-(3- chlorophenyl)ethyl]- 1-(1-ethyl-1H- pyrazol-4- yl)pyridazin- 4(1H)-one | Calc'd 329, found 329 |

### Scheme #5

### Examples #249 and 250

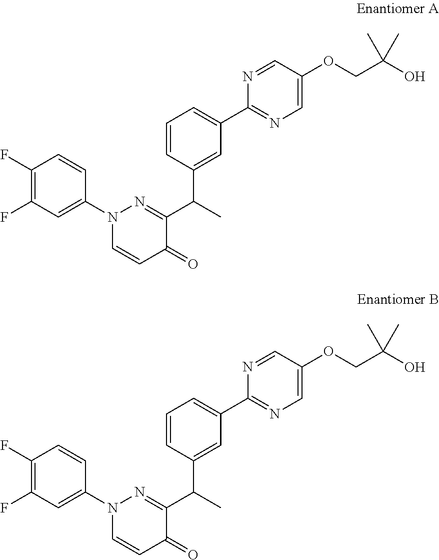

### 1-(3,4-Difluorophenyl)-3-[(1S or R)-1-{3-[5-(2-hydroxy-2-methylpropoxy)pyrimidin-2-yl]phenyl}ethyl]pyridazin-4(1H)-one (Enantiomer A) and 1-(3,4-difluorophenyl)-3-[(1R or S)-1-{3-[5-(2-hydroxy-2-methylpropoxy)pyrimidin-2-yl]phenyl}ethyl]pyridazin-4(1H)-one (Enantiomer B)

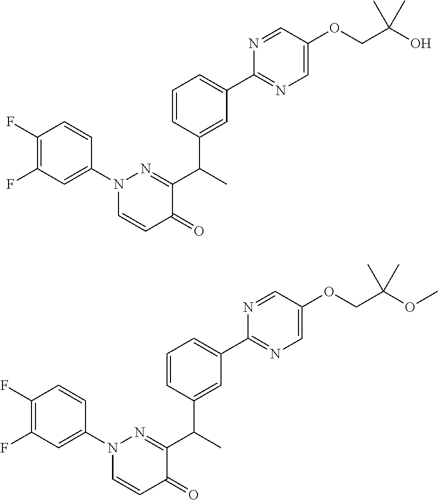

### Step 1. 1-(3,4-Difluorophenyl)-3-(1-{3-[5-(2-hydroxy-2-methylpropoxy)pyrimidin-2-yl]phenyl}ethyl)pyridazin-4(1H)-one and 1-(3,4-difluorophenyl)-3-(1-{3-[5-(2-methoxy-2-methylpropoxy)pyrimidin-2-yl]phenyl}ethyl)pyridazin-4(1H)-one

To a solution of 1-(3,4-difluorophenyl)-3-{3-[5-(2-hydroxy-2-methylpropoxy)pyrimidin-2-yl]benzyl}pyridazin-4(1H)-one (Example #360, 405 mg, 0.872 mmol) in DMF (4 mL) at 0° C. was added iodomethane (0.082 mL, 1.30 mmol) followed by NaH (60 wt %, 52 mg, 1.30 mmol). The reaction mixture was stirred at 0° C. for 30 minutes. Subsequently, saturated NaHCO₃ was added and the products extracted into EtOAc (3×). The combined organic extracts were dried-over Na₂SO₄, filtered and concentrated in vacuo. Purification of the residue by flash chromatography (MPLC, 0-15% MeOH-DCM) gave rac-1-(3,4-difluorophenyl)-3-(1-{3-[5-(2-hydroxy-2-methylpropoxy)pyrimidin-2-yl]phenyl}ethyl)pyridazin-4(1H)-one and rac-1-(3,4-di-fluorophenyl)-3-(1-{3-[5-(2-methoxy-2-methylpropoxy)pyrimidin-2-yl]phenyl}ethyl)pyridazin-4(1H)-one.

LRMS (ESI) calc'd for C26H25F2N4O3 [M+H]⁺: 479. Found: 479.

LRMS (ESI) calc'd for C27H27F2N4O3 [M+H]⁺: 493. Found: 493.

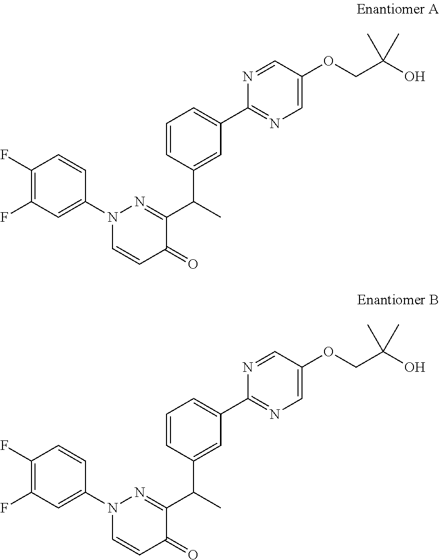

### Step 2. 1-(3,4-Difluorophenyl)-3-[(1S or R)-1-{3-[5-(2-hydroxy-2-methylpropoxy)pyrimidin-2-yl]phenyl}ethyl]pyridazin-4(1H)-one (Enantiomer A) and 1-(3,4-difluorophenyl)-3-[(1R or S)-1-{3-[5-(2-hydroxy-2-methylpropoxy)pyrimidin-2-yl]phenyl}ethyl]pyridazin-4(1H)-one (Enantiomer B)

rac-1-(3,4-Difluorophenyl)-3-(1-{3-[5-(2-hydroxy-2-methylpropoxy)pyrimidin-2-yl]phenyl}ethyl)pyridazin-4(1H)-one (200 mg, 0.418 mmol) was resolved by SFC (Berger Multigram II SFC, column: Chiral Technology AD-H 2.1×25 cm, 5 μM, mobile phase: 45% methanol/55% CO_{2(I)}, flow rate: 70 mL/min, 5.5 min run time) to give 1-(3,4-difluorophenyl)-3-[(1S or R)-1-{3-[5-(2-hydroxy-2-methylpropoxy)pyrimidin-2-yl]phenyl}ethyl]pyridazin-4(1H)-one (Enantiomer A, Example #249) and 1-(3,4-difluorophenyl)-3-[(1R or S)-1-{3-[5-(2-hydroxy-2-methylpropoxy)pyrimidin-2-yl]phenyl}ethyl]pyridazin-4(1H)-one (Enantiomer B, Example #250).

### Example #249

LRMS (ESI) calc'd for C26H25F2N4O3 [M+H]⁺: 479. Found: 479.

### Example #250

LRMS (ESI) calc'd for C26H25F2N4O3 [M+H]⁺: 479. Found: 479.

### Scheme #5

### Examples #251 and 252

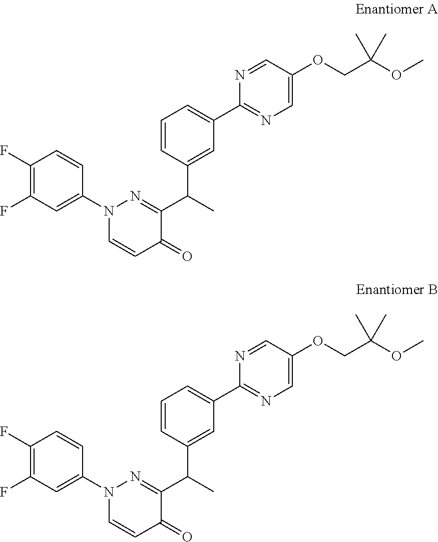

### 1-(3,4-Difluorophenyl)-3-[(1S or R)-1-{3-[5-(2-methoxy-2-methylpropoxy)pyrimidin-2-yl]phenyl}ethyl]pyridazin-4(1H)-one (Enantiomer A) and 1-(3,4-difluorophenyl)-3-[(1R or S)-1-{3-[5-(2-methoxy-2-methylpropoxy)pyrimidin-2-yl]phenyl}ethyl]pyridazin-4(1H)-one (Enantiomer B)

### Step 1. 1-(3,4-Difluorophenyl)-3-[(1S or R)-1-{3-[5-(2-methoxy-2-methylpropoxy)pyrimidin-2-yl]phenyl}ethyl]pyridazin-4(1H)-one (Enantiomer A) and 1-(3,4-difluorophenyl)-3-[(1R or S)-1-{3-[5-(2-methoxy-2-methylpropoxy)pyrimidin-2-yl]phenyl}ethyl]pyridazin-4(1H)-one (Enantiomer B)

rac-1-(3,4-Difluorophenyl)-3-(1-{3-[5-(2-methoxy-2-methylpropoxy)pyrimidin-2-yl]phenyl}ethyl)pyridazin-4(1H)-one (Examples #249-250 Step 1, 196 mg, 0.398 mmol) was resolved by SFC (Berger Multigram II SFC, column: Chiral Technology AD-H 2.1×25 cm, 5 μM, mobile phase: 45% methanol/55% CO_{2(I)}, flow rate: 70 mL/min, 5.5 min run time) to give 1-(3,4-difluorophenyl)-3-[(1S or R)-1-{3-[5-(2-methoxy-2-methylpropoxy)pyrimidin-2-yl]phenyl}ethyl]pyridazin-4(1H)-one (Enantiomer A, Example #251) and 1-(3,4-difluorophenyl)-3-[(1R or S)-1-{3-[5-(2-methoxy-2-methylpropoxy)pyrimidin-2-yl]phenyl}ethyl]pyridazin-4(1H)-one (Enantiomer B, Example #252).

### Example #251

LRMS (ESI) calc'd for C27H27F2N4O3 [M+H]⁺: 493. Found: 493.

### Example #252

LRMS (ESI) calc'd for C27H27F2N4O3 [M+H]⁺: 493. Found: 493.

### Scheme 5

### Example #253

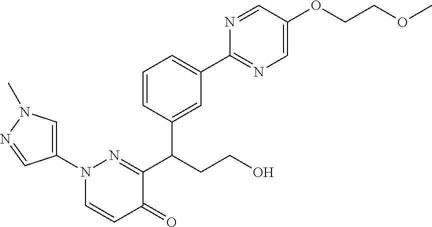

### rac-3-(3-Hydroxy-1-{3-[5-(2-methoxyethoxy)pyrimidin-2-yl]phenyl}propyl)-1-(1-methyl-1H-pyrazol-4-yl)pyridazin-4(1H)-one

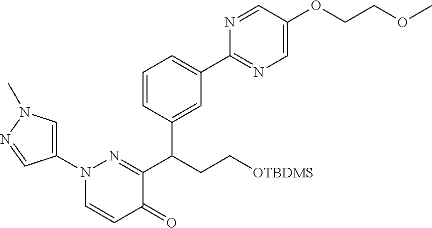

### Step 1. rac-3-{1-[3-(5-Ethoxypyrimidin-2-yl)phenyl]ethyl}-1-(1-methyl-1H-pyrazol-4-yl)pyridazin-4(1H)-one

3-{3-[5-(2-Methoxyethoxy)pyrimidin-2-yl]benzyl}-1-(1-methyl-1H-pyrazol-4-yl)pyridazin-4(1H)-one (Example #4, 131 mg, 0.313 mmol) and (2-bromoethoxy)-tert-butyldimethylsilane (0.20 mL, 0.939 mmol) were taken up in DMF (0.8 mL)/THF (0.8 mL). Sodium hydride (60 wt %, 38 mg, 0.94 mmol) was added and the resulting mixture stirred at room temperature for 3 hours. Saturated NH₄Cl was added and the products extracted into EtOAc (X2). The combined organic extracts were washed with brine, dried over Na₂SO₄, filtered and concentrated in vacuo. Purification of the residue by flash chromatography (MPLC, 0-15% MeOH-EtOAc) gave rac-3-(3-{[tert-butyl(dimethyl)silyl]oxy}-1-{3-[5-(2-methoxyethoxy)pyrimidin-2-yl]phenyl}propyl)-1-(1-methyl-1H-pyrazol-4-yl)pyridazin-4(1H)-one as a yellow gum.

LRMS (ESI) calc'd for C30H41N6O4Si [M+H]⁺: 577. Found: 577.

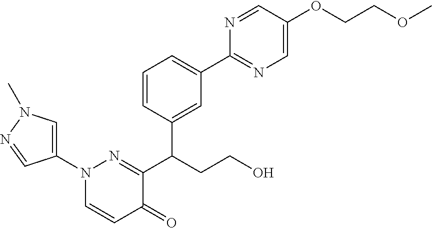

### Step 2. rac-3-(3-Hydroxy-1-{3-[5-(2-methoxyethoxy)pyrimidin-2-yl]phenyl}propyl)-1-(1-methyl-1H-pyrazol-4-yl)pyridazin-4(1H-one

rac-3-(3-{[tert-Butyl(dimethyl)silyl]oxy}-1-{3-[5-(2-methoxyethoxy)pyrimidin-2-yl]phenyl}propyl)-1-(1-methyl-1H-pyrazol-4-yl)pyridazin-4(1H)-one (109 mg, 0.189 mmol) was stirred in 1% conc. HCl in EtOH (1.9 mL) at room temperature for 3 hours. The solvent was removed in vacuo, saturated NaHCO₃ was added and the products extracted into 10% MeOH-DCM (X3). The combined organic extracts were dried over Na₂SO₄, filtered and concentrated in vacuo. Purification of the residue by flash chromatography (MPLC, 0-15% MeOH-EtOAc) gave rac-3-(3-hydroxy-1-{3-[5-(2-methoxyethoxy)pyrimidin-2-yl]phenyl}propyl)-1-(1-methyl-1H-pyrazol-4-yl)pyridazin-4(1H)-one as a white solid.

LRMS (ESI) calc'd for C24H27N6O4 [M+H]⁺: 463. Found: 463.

### Scheme 5

### Examples #254

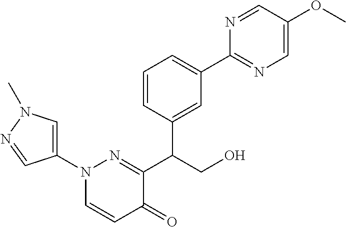

### rac-3-{2-Hydroxy-1-[3-(5-methoxypyrimidin-2-yl)phenyl]ethyl}-1-(1-methyl-1H-pyrazol-4-yl)pyridazin-4(1H)-one

### Step 1. rac-3-{2-Hydroxy-1-[3-(5-methoxypyrimidin-2-yl)phenyl]ethyl}-1-(1-methyl-1H-pyrazol-4-yl)pyridazin-4(1H)-one

3-[3-(5-Methoxypyrimidin-2-yl)benzyl]-1-(1-methyl-1H-pyrazol-4-yl)pyridazin-4(1H)-one (Example #5, 118 mg, 0.315 mmol) and SEM-C (0.112 mL, 0.630 mmol) were taken up in DMF (1.5 mL)/THF (1.5 mL). Sodium hydride (60 wt %, 38 mg, 0.95 mmol) was added and the resulting mixture stirred at room temperature for 3 hours. Saturated NH₄Cl was added and the products extracted into EtOAc (x2). The combined organic extracts were washed with brine, dried over Na₂SO₄, filtered and concentrated in vacuo. Purification of the residue by flash chromatography (MPLC, 0-10% MeOH-DCM) gave rac-3-{2-hydroxy-1-[3-(5-methoxypyrimidin-2-yl)phenyl]ethyl}-1-(1-methyl-1H-pyrazol-4-yl)pyridazin-4(1H)-one as a colorless gum.

LRMS (ESI) calc'd for C21H21N6O3 [M+H]⁺: 405. Found: 405.

### Scheme 5

### Examples #255 and 256

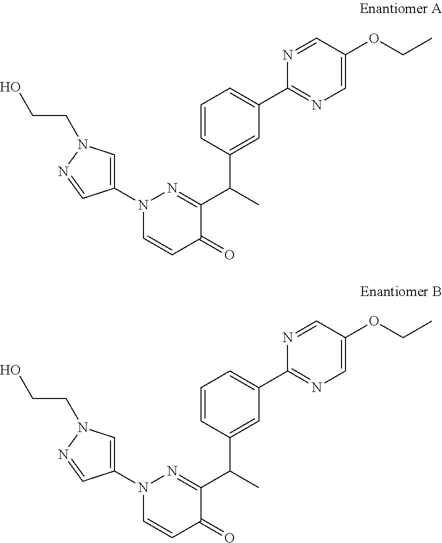

### 3-{(1S or R)-1-[3-(5-Ethoxypyrimidin-2-yl)phenyl]ethyl}-1-[1-(2-hydroxyethyl)-1H-pyrazol-4-yl]pyridazin-4(1H)-one and 3-{(1R or S)-1-[3-(5-ethoxypyrimidin-2-yl)phenyl]ethyl}-1-[1-(2-hydroxyethyl)-1H-pyrazol-4-yl]pyridazin-4(1H)-one

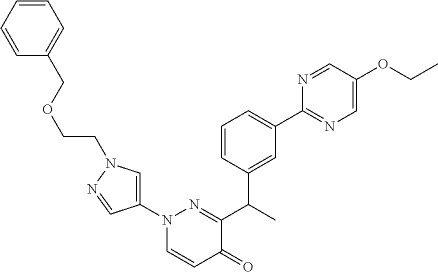

### Step-1. rac-1-{1-[2-(Benzyloxy)ethyl]-1H-pyrazol-4-yl}-3-{1-[3-(5-ethoxypyrimidin-2-yl)phenyl]ethyl}pyridazin-4(1H)-one

rac-1-{1-[2-(Benzyloxy)ethyl]-1H-pyrazol-4-yl}-3-{1-[3-(5-ethoxypyrimidin-2-yl)phenyl]ethyl}pyridazin-4(1H)-one was prepared from 1-{1-[2-(benzyloxy)ethyl]-1H-pyrazol-4-yl}-3-[3-(5-ethoxypyrimidin-2-yl)benzyl]pyridazin-4(1H)-one (Intermediate #111) according to the procedure described for rac-3-{1-[3-(5-ethoxypyrimidin-2-yl)phenyl]ethyl}-1-(1-methyl-1H-pyrazol-4-yl)pyridazin-4(1H)-one (Examples #201-202 Step 1).

LRMS (ESI) calc'd for C30H31N6O3 [M+H]⁺: 523. found 523.

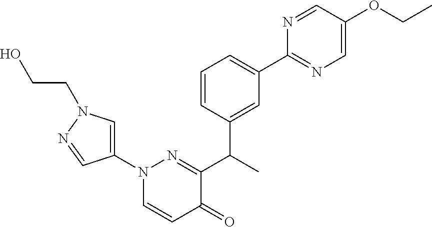

### Step 2. rac-3-{1-[3-(5-Ethoxypyrimidin-2-yl)phenyl]ethyl}-1-[1-(2-hydroxyethyl)-1H-pyrazol-4-yl]pyridazin-4(1H)-one

rac-3-{1-[3-(5-Ethoxypyrimidin-2-yl)phenyl]ethyl}-1-[1-(2-hydroxyethyl)-1H-pyrazol-4-yl]pyridazin-4(1H)-one was prepared was from rac-1-{1-[2-(benzyloxy)ethyl]-1H-pyrazol-4-yl}-3-{1-[3-(5-ethoxypyrimidin-2-yl)phenyl]ethyl}pyridazin-4(1H)-one (86 mg, 0.165 mmol) according to the procedure described for 3-[3-(5-ethoxypyrimidin-2-yl)benzyl]-1-[1-(2-hydroxyethyl)-1H-pyrazol-4-yl]pyridazin-4(1H)-one (Example #193).

LRMS (ESI) calc'd for C23H25N6O3 [M+H]⁺: 433. found 433.

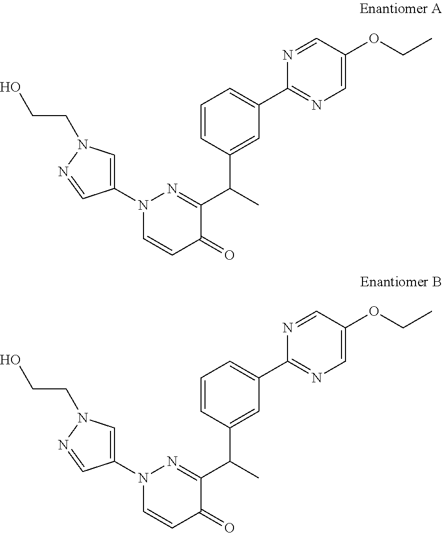

### Step 3. 3-{(1S or R)-1-[3-(5-Ethoxypyrimidin-2-yl)phenyl]ethyl}-1-[1-(2-hydroxyethyl)-1H-pyrazol-4-yl]pyridazin-4(1H)-one (Enantiomer A) and 3-{(1R or S)-1-[3-(5-ethoxypyrimidin-2-yl)phenyl]ethyl}-1-[1-(2-hydroxyethyl)-1H-pyrazol-4-yl]pyridazin-4(1H)-one (Enantiomer B)

rac-3-{1-[3-(5-Ethoxypyrimidin-2-yl)phenyl]ethyl}-1-[1-(2-hydroxyethyl)-1H-pyrazol-4-yl]pyridazin-4(1H)-one (30 mg, 0.069 mmol) was resolved by SFC (Berger Multigram II SFC, column: Chiral Technology AS-H 2.1×25 cm, 5 μM, mobile phase: 40% methanol/60% CO_{2(I)}, flow rate: 70 mL/min, 4.5 min. run time) to give 3-{(1S or R)-1-[3-(5-ethoxypyrimidin-2-yl)phenyl]ethyl}-1-[1-(2-hydroxyethyl)-1 H-pyrazol-4-yl]pyridazin-4(1H)-one (Enantiomer A, Example #255) and 3-{(1R or S)-1-[3-(5-ethoxypyrimidin-2-yl)phenyl]ethyl}-1-[1-(2-hydroxyethyl)-1H-pyrazol-4-yl]pyridazin-4(1H)-one (Enantiomer B, Example #256).

### Example #255

LRMS (ESI) calc'd for C23H25N6O3 [M+H]⁺: 433. found 433.

### Example #256

LRMS (ESI) calc'd for C23H25N6O3 [M+H]⁺: 433. found 433.

### Scheme 5

### Examples #257 and 258

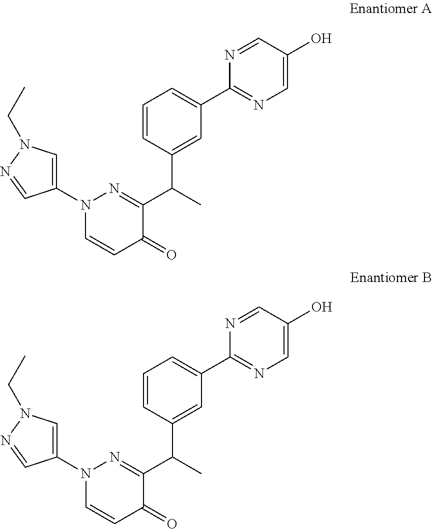

### 1-(1-Ethyl-1H-pyrazol-4-yl)-3-({(1S or R)-1-[3-(5-hydroxypyrimidin-2-yl)phenyl]ethyl}pyridazin-4(1H)-one (Enantiomer A) and 1-(1-ethyl-1H-pyrazol-4-yl)-3-{(1R or S)-1-[3-(5-Hydroxypyrimidin-2-yl)phenyl]ethyl}pyridazin-4(1H)-one (Enantiomer B)

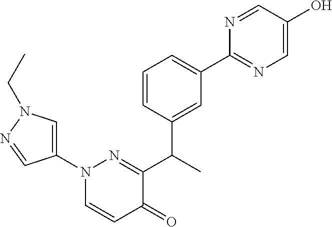

### Step 1. rac-1-(1-Ethyl-1H-pyrazol-4-yl)-3-{1-[3-(5-hydroxypyrimidin-2-yl)phenyl]ethyl}pyridazin-4(1H)-one

rac-3-(1-{3-[5-(Benzyloxy)pyrimidin-2-yl]phenyl}ethyl)-1-(1-ethyl-1H-pyrazol-4-yl)pyridazin-4(1H)-one (racemic mixture of Examples #228 and 229, 4.70 g, 9.82 mmol) was stirred in 33% HBr in AcOH (47 mL) at room temperature overnight. Most of the solvent was removed in vacuo and the residual oil neutralized with 1 N NaOH. The products were extracted into EtOAc (x2) followed by 10% MeOH-DCM (x3). The majority of the product remained in the aqueous phase. The aqueous phase was concentrated in vacuo and the residue suspended in 10% MeOH-DCM. After stirring for 30 minutes, the insoluble material was removed by filtering through Celite and the filtrate concentrated in vacuo. The residue was taken up in hot EtOAc with a minimum volume of MeOH and the mixture filtered once more through Celite. The filtrate was concentrated in vacuo and the residue purified by flash chromatography (MPLC, 0-20% MeOH-DCM) to give rac-1-(1-ethyl-1H-pyrazol-4-yl)-3-{1-[3-(5-hydroxypyrimidin-2-yl)phenyl]ethyl}pyridazin-4(1H)-one as an off-white solid.

LRMS (ESI) calc'd for C21H21N6O2 [M+H]⁺: 389. Found: 389.

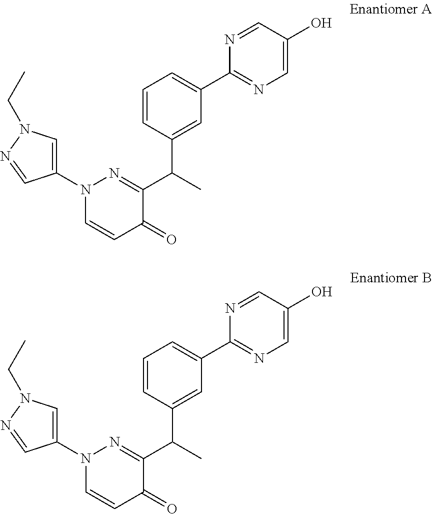

### Step 2. 1-(1-Ethyl-1H-pyrazol-4-yl)-3-{(1S or R)-1-[3-(5-hydroxypyrimidin-2-yl)phenyl]ethyl}pyridazin-4(1H)-one (Enantiomer A) and 1-(1-ethyl-1H-pyrazol-4-yl)-3-{(1R or S)-1-[3-(5-hydroxypyrimidin-2-yl)phenyl]ethyl}pyridazin-4(1H)-one (Enantiomer B)

rac-1-(1-Ethyl-1H-pyrazol-4-yl)-3-{1-[3-(5-hydroxypyrimidin-2-yl)phenyl]ethyl}pyridazin-4(1H)-one (2.4 g, 6.18 mmol) was resolved by SFC (column: ChiralPak IA 5×25 cm, mobile phase: 2-8% methanol/72% CO_{2(I)}, flow rate: 240 mL/min) to give 1-(1-ethyl-1H-pyrazol-4-yl)-3-{(1S or R)-1-[3-(5-hydroxypyrimidin-2-yl)phenyl]ethyl}pyridazin-4(1H)-one (Enantiomer A, Example #257) and 1-(1-ethyl-1H-pyrazol-4-yl)-3-{(R or S)-1-[3-(5-hydroxypyrimidin-2-yl)phenyl]ethyl}pyridazin-4(1H)-one (Enantiomer B, Example #258).

### Example #257

LRMS (ESI) calc'd for C21-H21N6O2 [M+H]⁺: 389. Found: 389.

### Example #258

LRMS (ESI) calc'd for C21H21N6O2 [M+H]⁺: 389. Found: 389.

### Scheme 5

### Intermediate #147

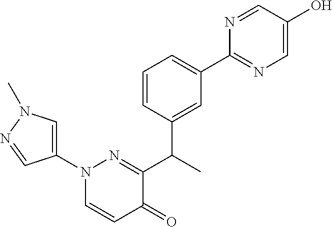

### rac-3-{1-[3-(5-Hydroxypyrimidin-2-yl)phenyl]ethyl}-1-(1-methyl-1H-pyrazol-4-yl)pyridazin-4(1H)-one

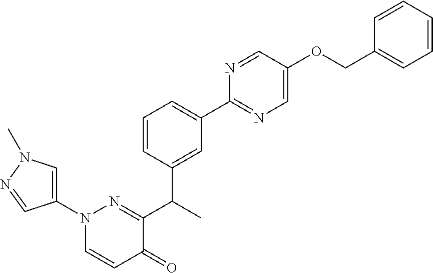

### Step 1. rac-3-(1-{3-[5-(benzyloxy)pyrimidin-2-yl]phenyl}ethyl)-1-(1-methyl-1H-pyrazol-4-yl)pyridazin-4(1H)-one

rac-3-(1-{3-[5-(Benzyloxy)pyrimidin-2-yl]phenyl}ethyl)-1-(1-methyl-1H-pyrazol-4-yl)pyridazin-4(1H)-one was prepared from 3-{3-[5-(benzyloxy)pyrimidin-2-yl]benzyl}-1-(1-methyl-1H-pyrazol-4-yl)pyridazin-4(1H)-one (Example #67) according to the procedure described for rac-3-{1-[3-(5-ethoxypyrimidin-2-yl)phenyl]ethyl}-1-(1-methyl-1H-pyrazol-4-yl)pyridazin-4(1H)-one (Examples #201-202 Step 1).

LRMS (ESI) calc'd for C27H25N6O2 [M+H]⁺: 465. Found: 465.

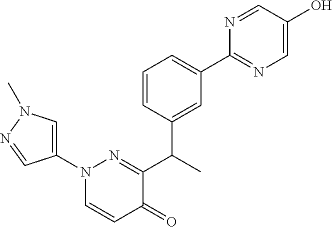

### Step 2. rac-3-{1-[3-(5-Hydroxypyrimidin-2-yl)phenyl]ethyl}-1-(1-methyl-1H-pyrazol-4-yl)pyridazin-4(1H)-one

rac-3-(1-{3-[5-(benzyloxy)pyrimidin-2-yl]phenyl}ethyl)-1-(1-methyl-1H-pyrazol-4-yl)pyridazin-4(1H)-one (960 mg, 2.07 mmol) was dissolved in EtOH (20 mL) and DCM (20 mL) and purged with nitrogen. Pd/C (5 wt %, 100 mg) was added and the reaction was stirred for 18 hours under H₂ (50 psi). The reaction mixture was filtered through Celite, rinsed with EtOH and DCM, and the solvent evaporated in vacuo to afford 3-{1-[3-(5-hydroxypyrimidin-2-yl)phenyl]ethyl}-1-(1-methyl-1H-pyrazol-4-yl)pyridazin-4(1H)-one as a yellow solid.

LRMS (ESI) calc'd for C20H19N6O2 [M+H]⁺: 375. Found: 375.

### Scheme 5

### Intermediates #148 and 149

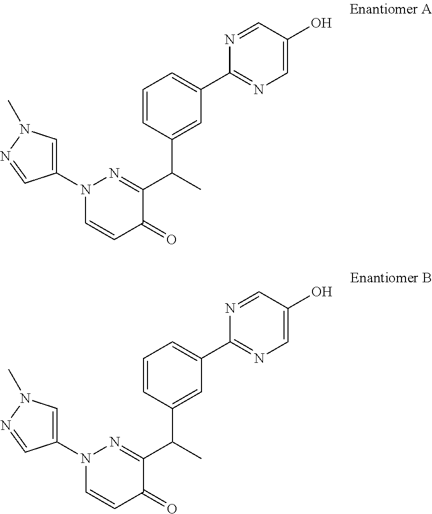

### 3-{(1S or R)-1-[3-(5-Hydroxypyrimidin-2-yl)phenyl]ethyl}-1-(1-methyl-1H-pyrazol-4-yl)pyridazin-4(1H)-one (Enantiomer A) and 3-{(1R or S)-1-[3-(5-hydroxypyrimidin-2-yl)phenyl]ethyl}-1-(1-methyl-1-pyrazol-4-yl)pyridazin-4(1H)-one (Enantiomer B)

### Step 1. 3-{(1S or R)-1-[3-(5-Hydroxypyrimidin-2-yl)phenyl]ethyl}-1-(1-methyl-1H-pyrazol-4-yl)pyridazin-4(1H)-one (Enantiomer A) and 3-{(1R or S)-1-[3-(5-hydroxypyrimidin-2-yl)phenyl]ethyl}-1-(1-methyl-1H-pyrazol-4-yl)pyridazin-4(1H)-one (Enantiomer B)

rac-3-{1-[3-(5-Hydroxypyrimidin-2-yl)phenyl]ethyl}-1-(1-methyl-1H-pyrazol-4-yl)pyridazin-4(1H)-one (Intermediate #147, 1.84 g, 4.92 mmol) was resolved by SFC (Berger Multigram II SFC, column: Chiral Technology AD-H 2.1×25 cm, 5 μM, mobile phase: 25% methanol/75% CO_{2(I)}, flow rate: 70 mL/min, 9 min run time) to give 3-{(1Ser R)-1-[3-(5-hydroxypyrimidin-2-yl)phenyl]ethyl}-1-(1-methyl-1H-pyrazol-4-yl)pyridazin-4(1H)-one (Enantiomer A, Intermediate #148) and 3-{(1R or S)-1-[3-(5-hydroxypyrimidin-2-yl)phenyl]ethyl}-1-(1-methyl-1H-pyrazol-4-yl)pyridazin-4(1H)-one (Enantiomer B, Intermediate #149).

### Intermediate #148

LRMS (ESI) calc'd for C20H19N6O2 [M+H]⁺: 375. Found: 375.

### Intermediate #149

LRMS (ESI) calc'd for C20H19N6O2 [M+H]⁺: 375. Found: 375.

### Scheme 6

### Example #259

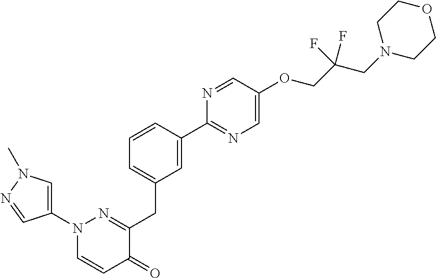

### 3-(3-{5-[2,2-Difluoro-3-(morpholin-4-yl)propoxy]pyrimidin-2-yl}benzyl)-1-(1-methyl-1H-pyrazol-4-yl)pyridazin-4(1H)-one

### Step 1. 3-(3-{5-[2,2-Difluoro-3-(morpholin-4-yl)propoxy]pyrimidin-2-yl}benzyl)-1-(1-methyl-1H-pyrazol-4-yl)pyridazin-4(1H)-one

3-[3-(5-Hydroxypyrimidin-2-yl)benzyl]-1-(1-methyl-1H-pyrazol-4-yl)pyridazin-4(1H)-one (Example #187, 50 mg, 0.14 mmol), 4-(2,2-difluoro-3-iodopropyl)morpholine (Intermediate #181, 81 mg, 0.28 mmol) and K₂CO₃ (38 mg, 0.28 mmol) were taken up in DMF (1 mL) and heated to 10° C. for 30-minutes under microwave irradiation (Biotage, Initiator). The reaction mixture was diluted with EtOAc and washed with brine. The organic layer was dried over Na₂SO₄, filtered and concentrated in vacuo while loading onto, silica. Purification of the residue by flash chromatography (MPLC, 0-10% MeOH-EtOAc) gave 3-(3-{5-[2,2-difluoro-3-(morpholin-4-yl)propoxy]pyrimidin-2-yl}benzyl)-1-(1-methyl-1H-pyrazol-4-yl)pyridazin-4(1H)-one as a white solid.

LRMS (ESI) calc'd for C26H28F2N7O3 [M+H]⁺: 524. Found: 524.

The following examples were prepared from Examples #187-189, 257-258 and Intermediates #139-141 according to Scheme 6 following a similar procedure described for Example #259, which can be achieved by those of ordinary skill in the art of organic synthesis. Note: Examples #296-299 were prepared from Example #258 and the racemic alkyl halide to generate a mixture of two diastereoisomers.

| | | | Exact |
|---|---|---|---|
| Exam- | | | Mass |
| ple | Structure | IUPAC Name | [M + H]⁺ |
| 260 | 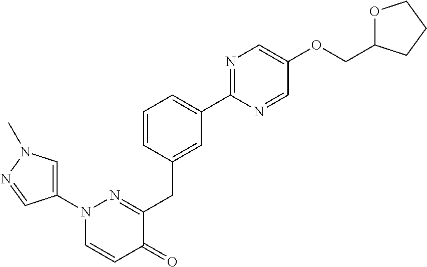 | rac-1-(1- methyl-1H- pyrazol-4-yl)-3-{3-[5- (tetrahydrofuran-2- ylmethoxy)pyrimidin-2- yl]benzyl}pyridazin- 4(1H)-one | Calc'd 445, found 445 |
| 261 | 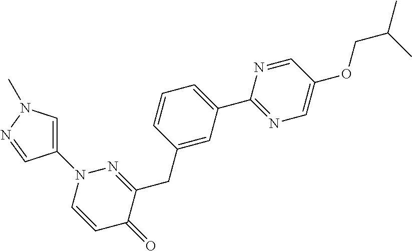 | 3-{3-[5-(2- methylpropoxy) pyrimidin-2- yl]benzyl}-1-(1- methyl-1H- pyrazol-4- yl)pyridazin- 4(1H)-one | Calc'd 417, found 417 |
| 262 | 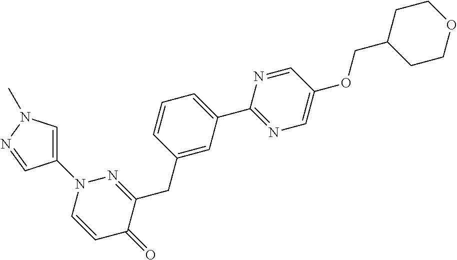 | 1-(1-methyl- 1H-pyrazol-4- yl)-3-{3-[5- (tetrahydro-2H- pyran-4- ylmethoxy)pyrimidin-2- yl]benzyl}pyridazin- 4(1H)-one | Calc'd 459, found 459 |
| 263 | 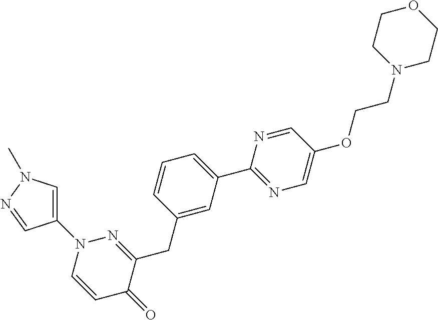 | 1-(1-methyl- 1H-pyrazol-4- yl)-3-{3-[5-(2- morpholin-4- ylethoxy)pyrimidin-2- yl]benzyl}pyridazin- 4(1H)-one | Calc'd 474, found 474 |
| 264 | 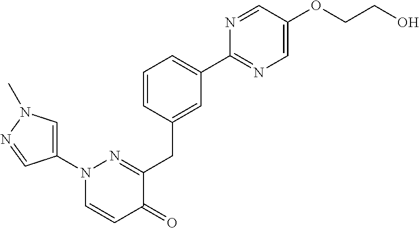 | 3-{3-[5-(2- hydroxyethoxy) pyrimidin-2- yl]benzyl}-1-(1- methyl-1H- pyrazol-4- yl)pyridazin- 4(1H)-one | Calc'd 405, found 405 |
| 265 | 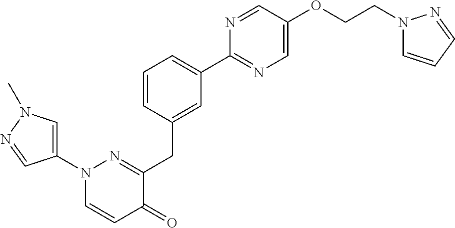 | 1-(1-methyl- 1H-pyrazol-4- yl)-3-(3-{5-[2- (1H-pyrazol-1- yl)ethoxy]pyrimidin-2- yl}benzyl)pyridazin- 4(1H)-one | Calc'd 455, found 455 |
| 266 | 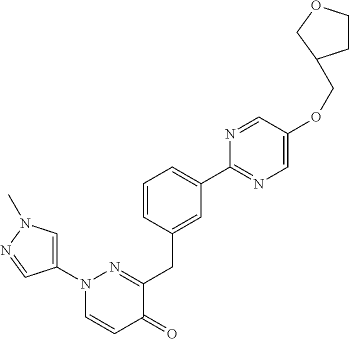 | rac-1-(1- methyl-1H- pyrazol-4-yl)-3- {3-[5- (tetrahydrofuran-3- ylmethoxy)pyrimidin-2- yl]benzyl}pyridazin- 4(1H)-one | Calc'd 445, found 445 |
| 267 | 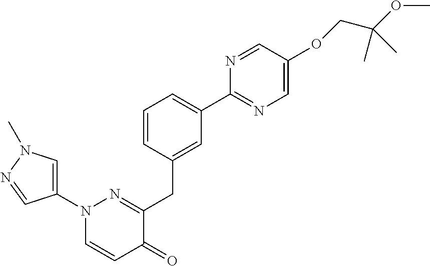 | 3-{3-[5-(2- methoxy-2- methylpropoxy) pyrimidin-2- yl]benzyl}-1-(1- methyl-1H- pyrazol-4- yl)pyridazin- 4(1H)-one | Calc'd 447, found 447 |
| 268 | 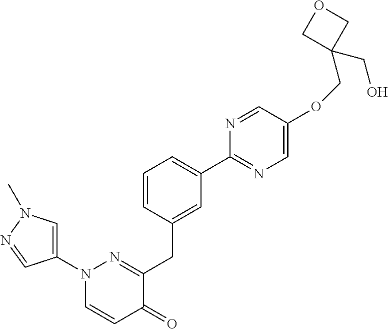 | 3-[3-(5-{[3- (hydroxymethyl) oxetan-3- yl]methoxy} pyrimidin-2- yl)benzyl]-1-(1- methyl-1H- pyrazol-4- yl)pyridazin- 4(1H)-one | Calc'd 461, found 461 |
| 269 | 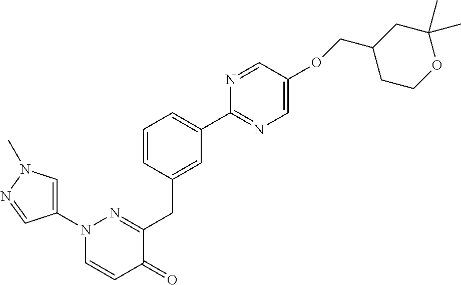 | rac-3-(3-{5-[(2,2- dimethyltetrahydro- 2H-pyran-4- yl)methoxy] pyrimidin-2- yl}benzyl)-1-(1- methyl-1H- pyrazol-4- yl)pyridazin- 4(1H)-one | Calc'd 487, found 487 |
| 270 | 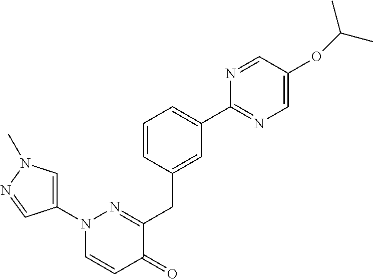 | 3-{3-[5-(1- methylethoxy) pyrimidin-2- yl]benzyl}-1-(1- methyl-1H- pyrazol-4- yl)pyridazin- 4(1H)-one | Calc'd 403, found 403 |
| 271 | 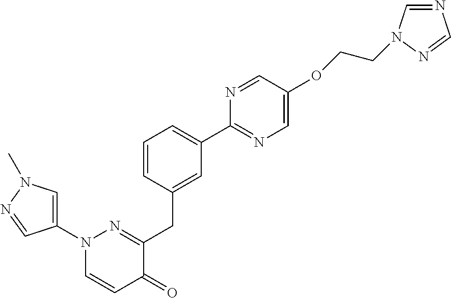 | 1-(1-methyl- 1H-pyrazol-4- yl)-3-(3-{5-[2- (1H-1,2,4- triazol-1- yl)ethoxy]pyrimidin-2- yl}benzyl)pyridazin- 4(1H)-one | Calc'd 456, found 456 |
| 272 | 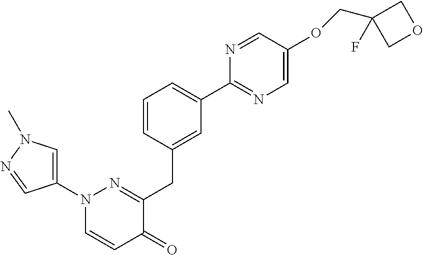 | 3-(3-{5-[(3- fluorooxetan-3- yl)methoxy]pyrimidin-2- yl}benzyl)-1-(1- methyl-1H- pyrazol-4- yl)pyridazin- 4(1H)-one | Calc'd 449, found 449 |
| 273 | 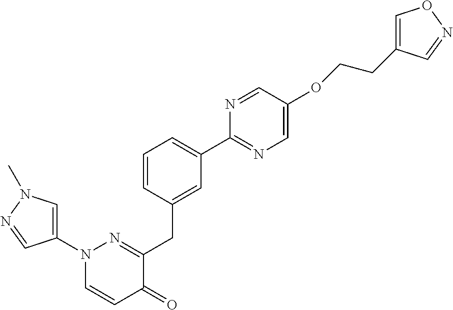 | 3-{3-[5-(2- isoxazol-4- ylethoxy)pyrimidin-2- yl]benzyl}-1-(1- methyl-1H- pyrazol-4- yl)pyridazin- 4(1H)-one | Calc'd 456, found 456 |
| 274 | 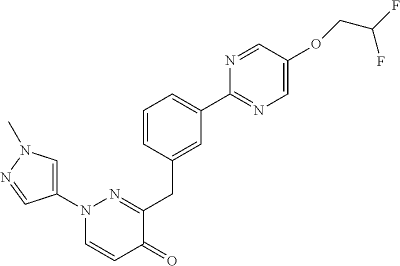 | 3-{3-[5-(2,2- difluoroethoxy) pyrimidin-2- yl]benzyl}-1-(1- methyl-1H- pyrazol-4- yl)pyridazin- 4(1H)-one | Calc'd 425, found 425 |
| 275 | 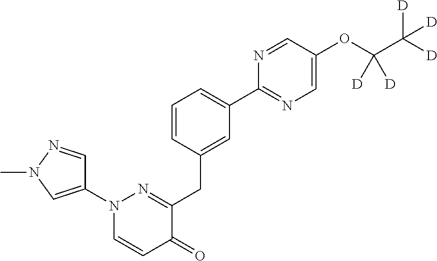 | 3-[3-(5- ethoxypyrimidin- 2-yl)benzyl]- 1-(1-methyl- 1H-pyrazol-4- yl)pyridazin- 4(1H)-one-d5 | Calc'd 394, found 394 |
| 276 | 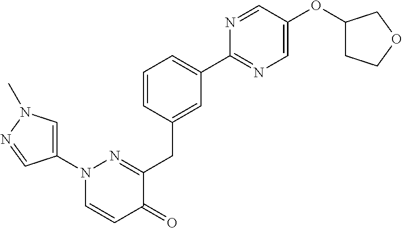 | rac-1-(1- methyl-1H- pyrazol-4-yl)-3- {3-[5- (tetrahydrofuran-3- yloxy)pyrimidin-2- yl]benzyl}pyridazin- 4(1H)-one | Calc'd 431, found 431 |
| 277 | 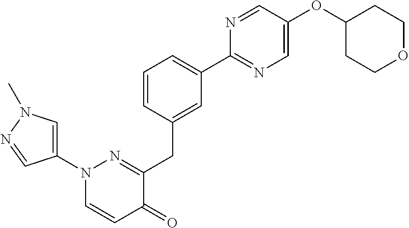 | 1-(1-methyl- 1H-pyrazol-4- yl)-3-{3-[5- (tetrahydro-2H- pyran-4- yloxy)pyrimidin-2- yl]benzyl}pyridazin- 4(1H)-one | Calc'd 445, found 445 |
| 278 | 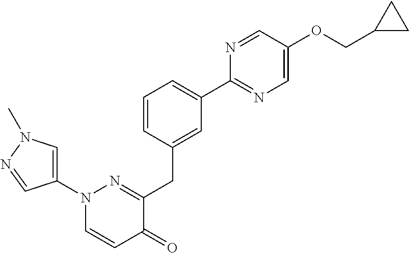 | 3-{3-[5- (cyclopropylmethoxy) pyrimidin- 2-yl]benzyl}-1- (1-methyl-1H- pyrazol-4- yl)pyridazin- 4(1H)-one | Calc'd 415, found 415 |
| 279 | 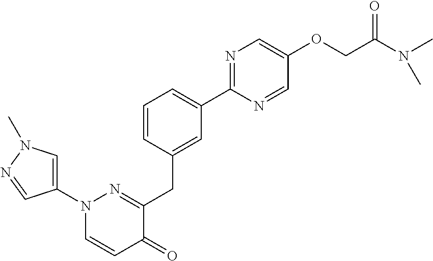 | N,N-dimethyl- 2-{[2-(3-{[1-(1- methyl-1H- pyrazol-4-yl)-4- oxo-1,4- dihydropyridazin-3- yl]methyl}phenyl) pyrimidin-5- yl]oxy}acetamide | Calc'd 446, found 446 |
| 280 | 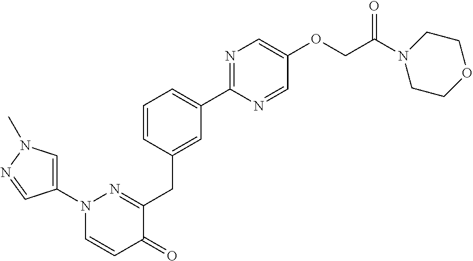 | 1-(1-methyl- 1H-pyrazol-4- yl)-3-{3-[5-(2- morpholin-4-yl-2- oxoethoxy)pyrimidin-2- yl]benzyl}pyridazin- 4(1H)-one | Calc'd 488, found 488 |
| 281 | 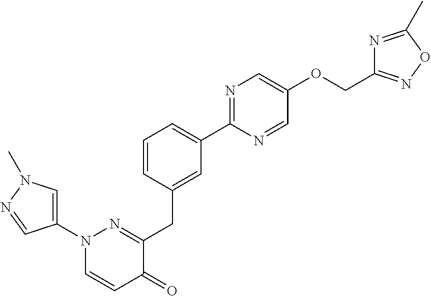 | 3-(3-{5-[(5- methyl-1,2,4- oxadiazol-3- yl)methoxy]pyrimidin-2- yl}benzyl)-1-(1- methyl-1H- pyrazol-4- yl)pridazin- 4(1H)-one | Calc'd 457, found 457 |
| 282 | 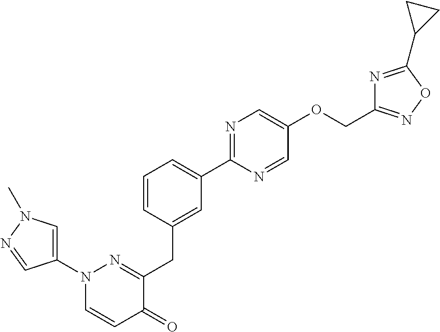 | 3-(3-{5-[(5- cyclopropyl- 1,2,4-oxadiazol-3- yl)methoxy]pyrimidin-2- yl}benzyl)-1-(1- methyl-1H- pyrazol-4- yl)pyridazin- 4(1H)-one | Calc'd 483, found 483 |
| 283 | 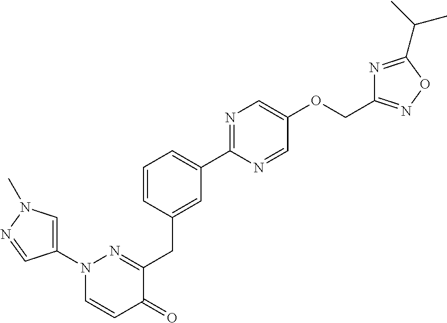 | 3-[3-(5-{[5-(1- methylethyl)- 1,2,4-oxadiazol-3- yl]methoxy} pyrimidin-2- yl)benzyl]-1-(1- methyl-1H- pyrazol-4- yl)pyridazin- 4(1H)-one | Calc'd 485, found 485 |
| 284 | 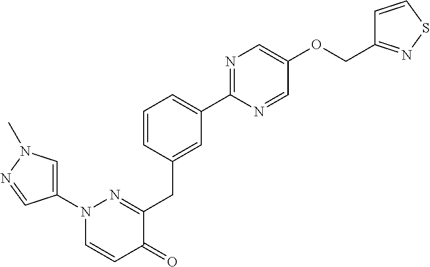 | 3-{3-[5- (isothiazol-3- ylmethoxy) pyrimidin-2- yl]benzyl}-1-(1- methyl-1H- pyrazol-4- yl)pyridazin- 4(1H)-one | Calc'd 458, found 458 |
| 285 | 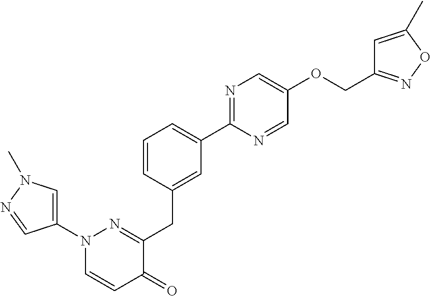 | 3-(3-{5-[(5- methylisoxazol-3- yl)methoxy]pyrimidin-2- yl}benzyl)-1-(1- methyl-1H- pyrazol-4- yl)pyridazin- 4(1H)-one | Calc'd 456, found 456 |
| 286 | 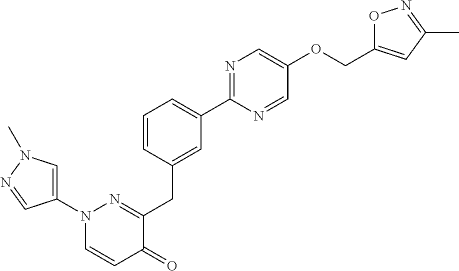 | 3-(3-{5-[(3- methylisoxazol-5- yl)methoxy]pyrimidin-2- yl}benzyl)-1-(1- methyl-1H- pyrazol-4- yl)pyridazin- 4(1H)-one | Calc'd 456, found 456 |
| 287 | 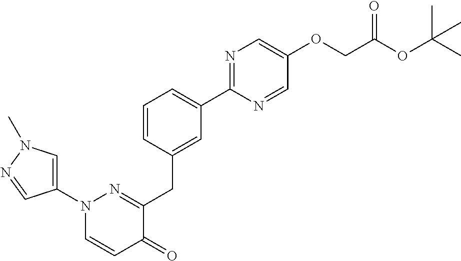 | tert-butyl [2-(3- {[1-(1-methyl- 1H-pyrazol-4- yl)-4-oxo-1,4- dihydropyridazin-3- yl]methyl}phenyl) pyrimidin-5- yl]oxyacetate | Calc'd 475, found 475 |
| 288 | 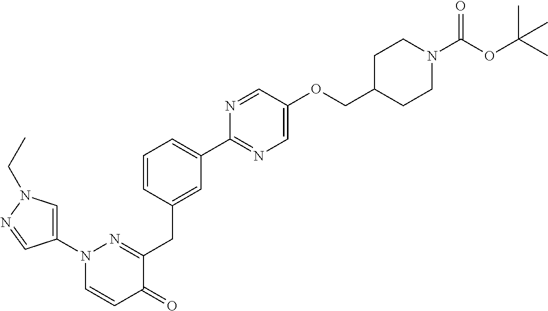 | tert-butyl 4- ({[2-(3-{[1-(1- ethyl-1H- pyrazol-4-yl)-4- oxo-1,4- dihydropyridazin-3- yl]methyl}phenyl) pyrimidin-5- yl]oxy}methyl) piperidine-1- carboxylate | Calc'd 572, found 572 |
| 289 | 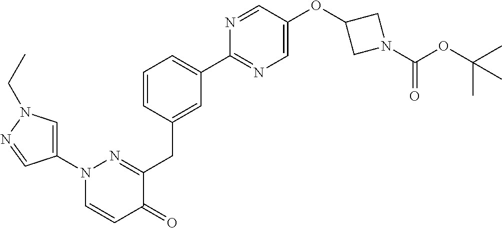 | tert-butyl 3-{[2- (3-{[1-(1-ethyl- 1H-pyrazol-4- yl)-4-oxo-1,4- dihydropyridazin-3- yl]methyl}phenyl) pyrimidin-5- yl]oxy}azetidine- 1-carboxylate | Calc'd 530, found 530 |
| 290 | 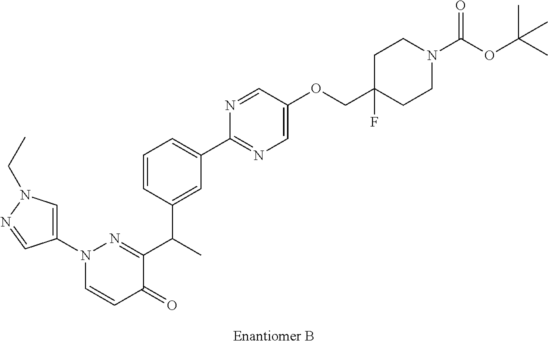 | tert-butyl 4- ({[2-(3-{(1R or S)-1-[1-(1- ethyl-1H pyrazol-4-yl)-4- oxo-1,4- dihydropyridazin-3- yl]ethyl}phenyl) pyrimidin-5- yl]oxy}methyl)-4- fluoropiperidine- 1-carboxylate | Calc'd 590, found 590 |
| 291 | 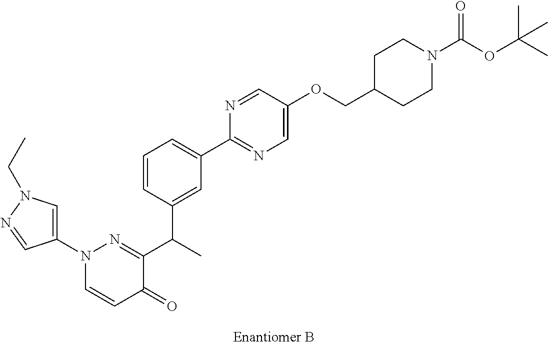 | tert-butyl 4- ({[2-(3-{(1R or S)-1-[1-(1- ethyl-1H- pyrazol-4-yl)-4- oxo-1,4- dihydropyridazin-3- yl]ethyl}phenyl) pyrimidin-5- yl]oxy}methyl) piperidine-1- carboxylate | Calc'd 604, found 604 |
| 292 | 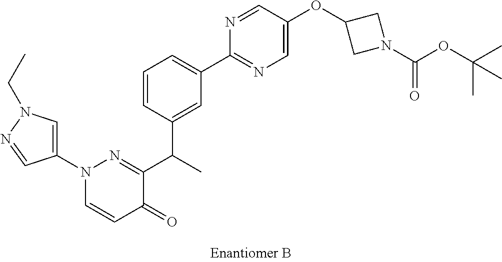 | tert-butyl 3-{[2- (3-{(1R or S)-1- [1-(1-ethyl-1H- pyrazol-4-yl)-4- oxo-1,4- dihydropyridazin-3- yl]ethyl}phenyl) pyrimidin-5- yl]oxy}azetidine- 1-carboxylate | Calc'd 544, found 544 |
| 293 | 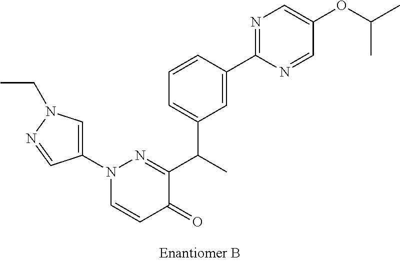 | 1-(1-ethyl-1H- pyrazol-4-yl)-3- [(1R or S)-1-{3-[5-(1- methylethoxy) pyrimidin-2- yl]phenyl}ethyl] pyridazin- 4(1H)-one | Calc'd 431, found 431 |
| 294 | 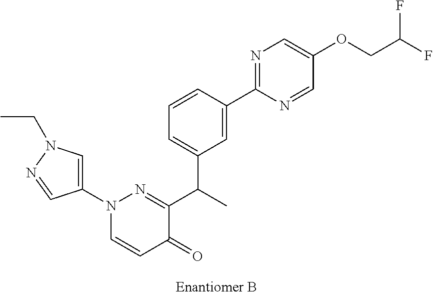 | 3-[(1R or S)-1- {3-[5-(2,2- difluoroethoxy) pyrimidin-2- yl]phenyl}ethyl]- 1-(1-ethyl-1H- pyrazol-4- yl)pyridazin- 4(1H)-one | Calc'd 453, found 453 |
| 295 | 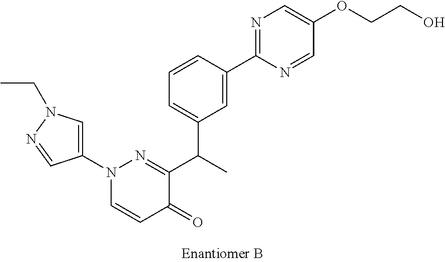 | 1-(1-ethyl-1H- pyrazol-4-yl)-3- [(1R or S)-1-{3-[5-(2- hydroxyethoxy) pyrimidin-2- yl]phenyl}ethyl] pyridazin- 4(1H)-one | Calc'd 433, found 433 |
| 296 | 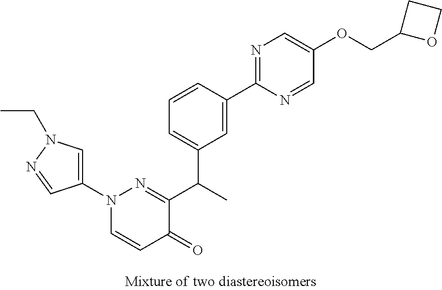 | 1-(1-ethyl-1H- pyrazol-4-yl)-3- [(1R or S)-1-{3- [5-(oxetan-2- ylmethoxy)primidin-2- yl]phenyl}ethyl] pyridazin- 4(1H)-one | Calc'd 459, found 459 |
| 297 | 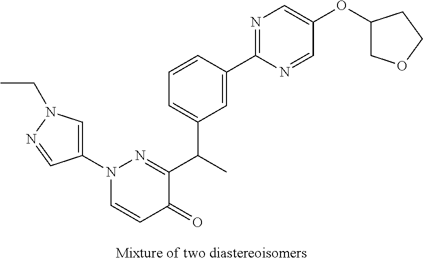 | 1-(1-ethyl-1H- pyrazol-4-yl)-3- [(1R or S)-1-{3-[5- (tetrahydrofuran-3- yloxy)pyrimidin-2- yl]phenyl}ethyl] pyridazin- 4(1H)-one | Calc'd 459, found 459 |
| 298 | 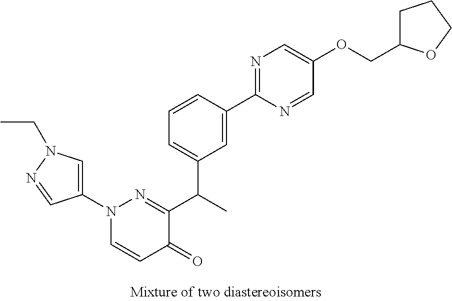 | 1-(1-ethyl-1H- pyrazol-4-yl)-3- [(1R or S)-1-{3-[5- (tetrahydrofuran-2- ylmethoxy)pyrimidin-2- yl]phenyl}ethyl] pyridazin- 4(1H)-one | Calc'd 473, found 473 |
| 299 | 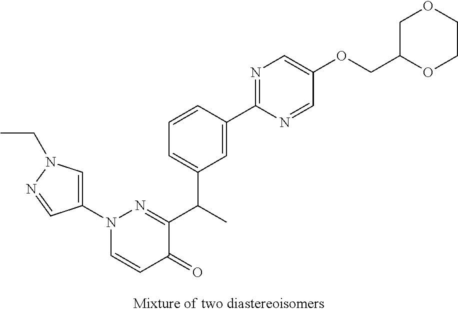 | 3-[(1R or S)-1- {3-[5-(1,4- dioxan-2- ylmethoxy)pyrimidin-2- yl]phenyl}ethyl]- 1-(1-ethyl-1H- pyrazol-4- yl)pyriazin- 4(1H)-one | Calc'd 489, found 489 |
| 300 | 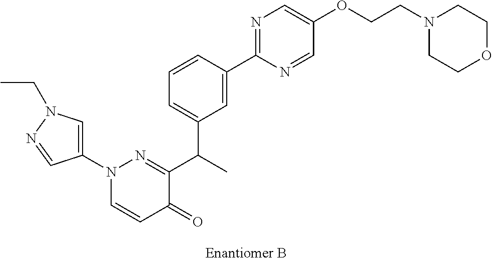 | 1-(1-ethyl-1H- pyrazol-4-yl)-3- [(1R or S)-1-{3-[5-(2- morpholin-4- ylethoxy)pyrimidin-2- yl]phenyl}ethyl] pyridazin- 4(1H)-one | Calc'd 502, found 502 |
| 301 | 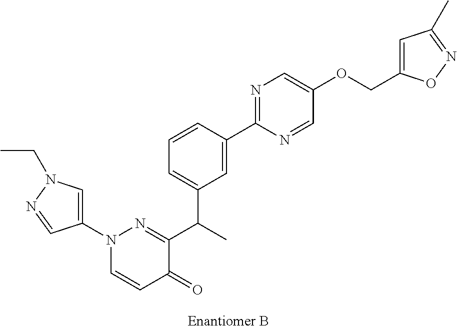 | 1-(1-ethyl-1H- pyrazol-4-yl)-3- [(1R or S)-1-(3- {5-[(3- methylisoxazol-5- yl)methoxy]pyrimidin-2- yl}phenyl)ethyl] pyridazin- 4(1H)-one | Calc'd 484, found 484 |
| 302 | 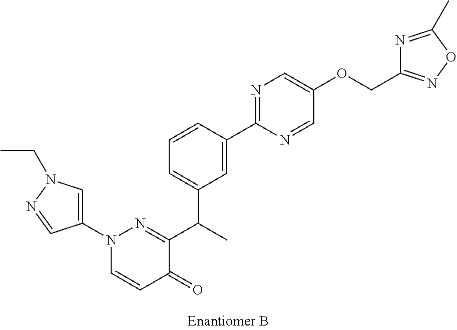 | 1-(1-ethyl-1H- pyrazol-4-yl)-3- [(1R or S)-1-(3- {5-[(5-methyl- 1,2,4-oxadiazol-3- yl)methoxy]pyrimidin-2- yl}phenyl)ethyl] pyridazin- 4(1H)-one | Calc'd 485, found 485 |
| 303 | 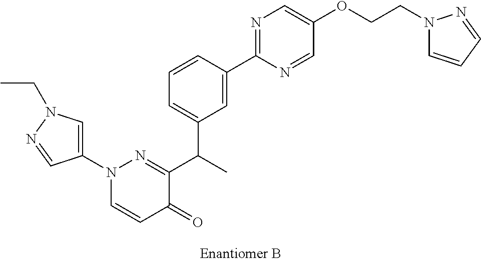 | 1-(1-ethyl-1H- pyrazol-4-yl)-3- [(1R or S)-1-(3- {5-[2-(1H- pyrazol-1- yl)ethoxy]pyrimidin-2- yl}phenyl)ethyl] pyridazin- 4(1H)-one | Calc'd 483, found 483 |
| 304 | 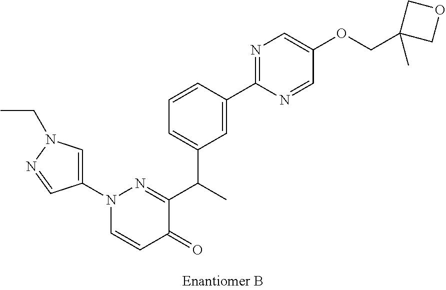 | 1-(1-ethyl-1H- pyrazol-4-yl)-3- [(1R or S)-1-(3- {5-[(3- methyloxetan-3- yl)methoxy]pyrimidin-2- yl}phenyl)ethyl] pyridazin- 4(1H)-one | Calc'd 473, found 473 |
| 305 | 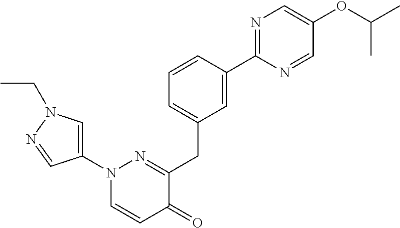 | 1-(1-ethyl-1H- pyrazol-4-yl)-3-[3-(5- isopropoxypyrimidin-2- yl)benzyl]pyridazin- 4(1H)-one | Calc'd 417, found 417 |
| 306 | 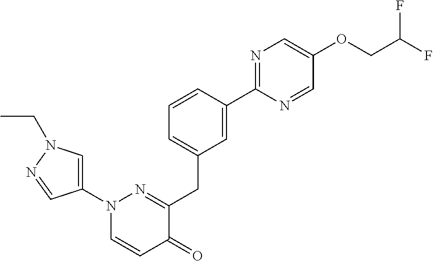 | 3-{3-[5-(2,2- difluoroethoxy) pyrimidin-2- yl]benzyl}-1-(1- ethyl-1H- pyrazol-4- yl)pyridazin- 4(1H)-one | Calc'd 439, found 439 |
| 307 | 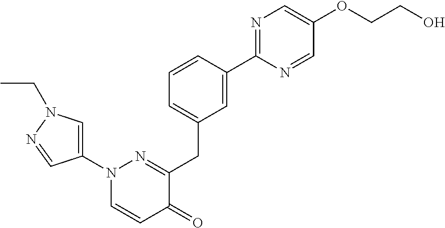 | 1-(1-ethyl-1H- pyrazol-4-yl)-3- {3-[5-(2- hydroxyethoxy) pyrimidin-2- yl]benzyl}pyridazin- 4(1H)-one | Calc'd 419, found 419 |
| 308 | 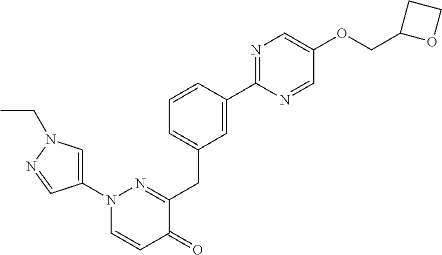 | rac-1-(1-ethyl- 1H-pyrazol-4- yl)-3-{3-[5- (oxetan-2- ylmethoxy)pyrimidin-2- yl]benzyl}pyridazin- 4(1H)-one | Calc'd 445, found 445 |
| 309 | 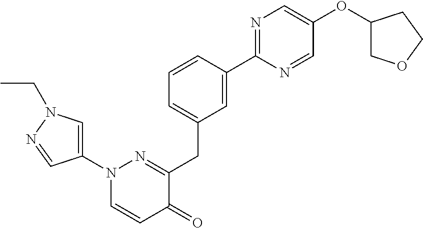 | rac-1-(1-ethyl- 1H-pyrazol-4- yl)-3-{3-[5- (tetrahydrofuran-3- yloxy)pyrimidin-2- yl]benzyl}pyridazin- 4(1H)-one | Calc'd 445, found 445 |
| 310 | 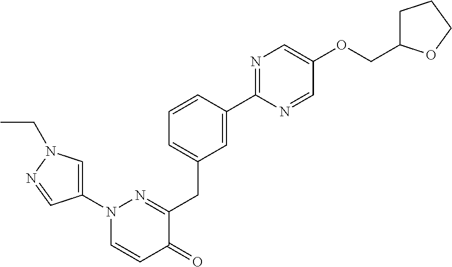 | rac-1-(1-ethyl- 1H-pyrazol-4- yl)-3-{3-[5- (tetrahydrofaran-2- ylmethoxy)pyrimidin-2- yl]benzyl}pyridazin- 4(1H)-one | Calc'd 459, found 459 |
| 311 | 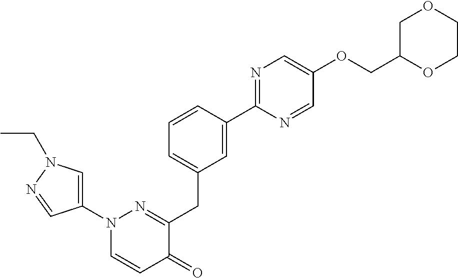 | rac-3-{3-[5- (1,4-dioxan-2- ylmethoxy)pyrimidin-2- yl]benzyl}-1-(1- ethyl-1H- pyrazol-4- yl)pyridazin- 4(1H)-one | Calc'd 475, found 475 |
| 312 | 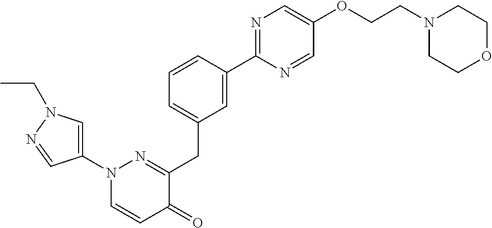 | 1-(1-ethyl-1H- pyrazol-4-yl)-3- {3-[5-(2- morpholin-4- ylethoxy)pyrimidin-2- yl]benzyl}pyridazin- 4(1H)-one | Calc'd 488, found 488 |
| 313 | 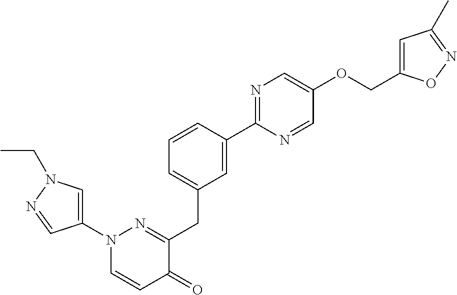 | 1-(1-ethyl-1H- pyrazol-4-yl)-3- (3-{5-[(3- methylisoxazol-5- yl)methoxy]primidin-2- yl}benzyl)pyridazin- 4(1H)-one | Calc'd 470, found 470 |
| 314 | 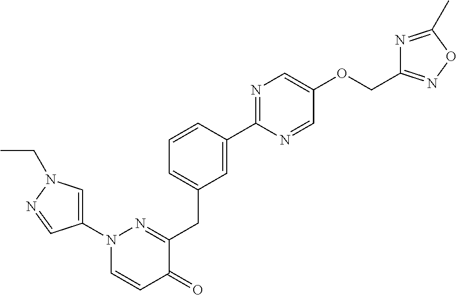 | 1-(1-ethyl-1H- pyrazol-4-yl)-3- (3-{5-[(5- methyl-1,2,4- oxadiazol-3- yl)methoxy]pyrimidin-2- yl}benzyl)pyridazin- 4(1H)-one | Calc'd 471, found 471 |
| 315 | 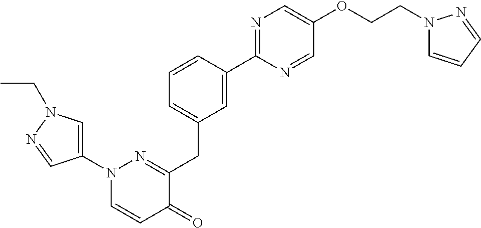 | 1-(1-ethyl-1H- pyrazol-4-yl)-3- (3-{5-[2-(1H- pyrazol-1- yl)ethoxy]pyrimidin-2- yl}benzyl)pyridazin- 4(1H)-one | Calc'd 469, found 469 |
| 316 | 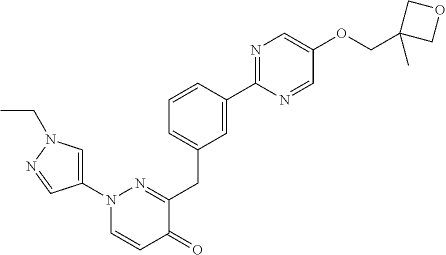 | 1-(1-ethyl-1H- pyrazol-4-yl)-3- (3-{5-[(3- methyloxetan-3- yl)methoxy]pyrimidin-2- yl}benzyl)pyridazin- 4(1H)-one | Calc'd 459, found 459 |
| 317 | 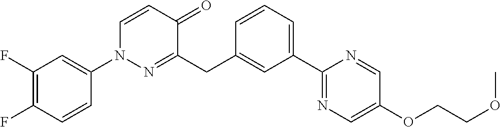 | 1-(3,4- difluorophenyl)- 3-{3-[5-(2- methoxyethoxy) pyrimidin-2- yl]benzyl}pyridazin- 4(1H)-one | Calc'd 451, found 451 |
| 318 | 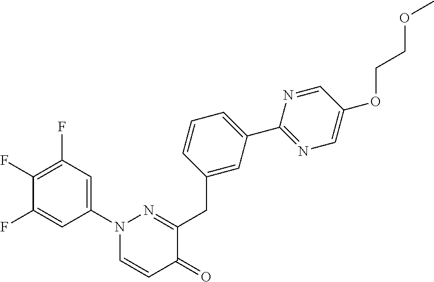 | 3-{3-[5-(2- methoxyethoxy) pyrimidin-2- yl]benzyl}-1- (3,4,5- trifluorophenyl) pyridazin- 4(1H)-one | Calc'd 469, found 469 |
| 319 | 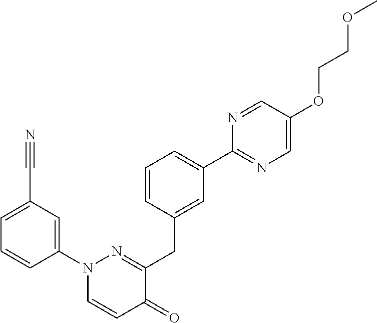 | 3-[3-{3-[5-(2- methoxyethoxy) pyrimidin-2- yl]benzyl}-4- oxopyridazin- 1(4H)- yl]benzonitrile | Calc'd 440, found 440 |
| 320 | 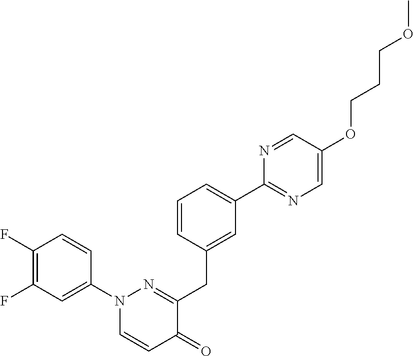 | 1-(3,4- difluorophenyl)- 3-{3-[5-(3- methoxypropoxy) pyrimidin-2- yl]benzyl}pyridazin- 4(1H)-one | Calc'd 465, found 465 |
| 321 | 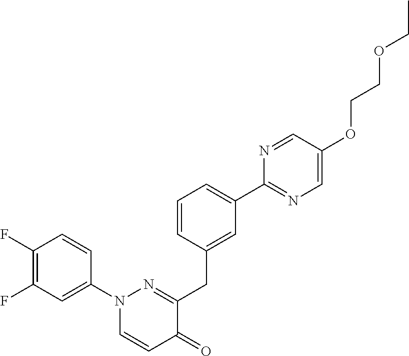 | 1-(3,4- difluorophenyl)- 3-{3-[5-(2- ethoxyethoxy) pyrimidin-2- yl]benzyl}pyridazin- 4(1H)-one | Calc'd 465, found 465 |
| 322 | 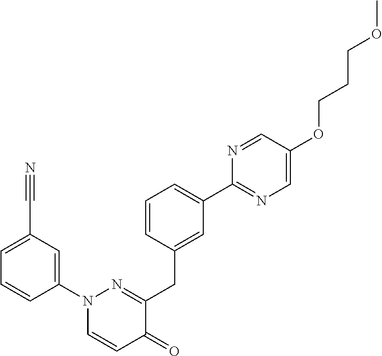 | 3-[3-{3-[5-(3- methoxypropoxy) pyrimidin-2- yl]benzyl}-4- oxopyridazin- 1(4H)- yl]benzonitrile | Calc'd 454, found 454 |
| 323 | 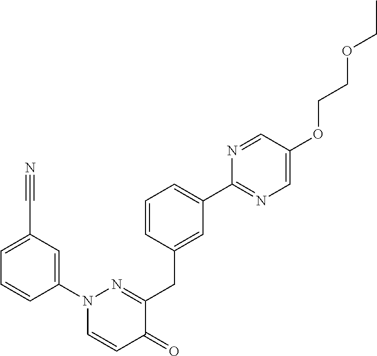 | 3-[3-{3-[5-(2- ethoxyethoxy) pyrimidin-2- yl]benzyl}-4- oxopyridazin- 1(4H)- yl]benzonitrile | Calc'd 454, found 454 |
| 324 | 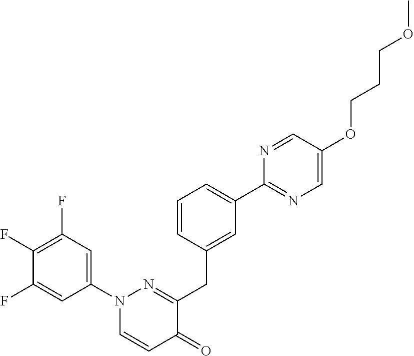 | 3-{3-[5-(3- methoxypropoxy) pyrimidin-2- yl]benzyl}-1- (3,4,5- trifluorophenyl) pyridazin- 4(1H)-one | Calc'd 483, found 483 |
| 325 | 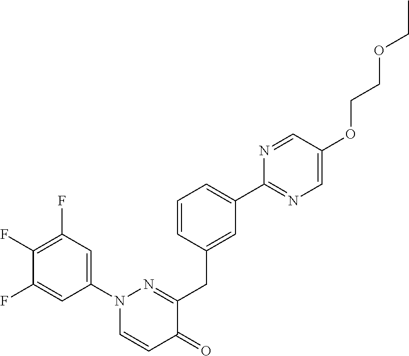 | 3-{3-[5-(2- ethoxyethoxy) pyrimidin-2- yl]benzyl}-1- (3,4,5- trifluorophenyl) pyridazin- 4(1H)-one | Calc'd 483, found 483 |
| 326 | 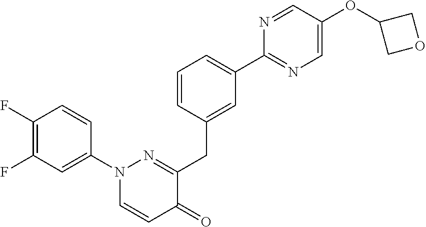 | 1-(3,4- difluorophenyl)- 3-{3-[5- (oxetan-3- yloxy)pyrimidin-2- yl]benzyl}pyridazin- 4(1H)-one | Calc'd 449, found 449 |
| 327 | 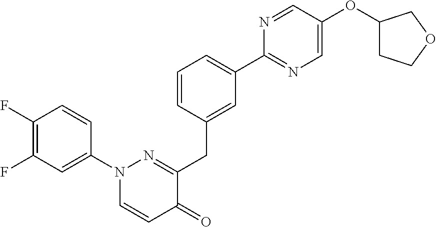 | rac-1-(3,4- difluorophenyl)- 3-{3-[5- (tetrahydrofuran-3- yloxy)pyrimidin-2- yl]benzyl}pyridazin- 4(1H)-one | Calc'd 463, found 463 |
| 328 | 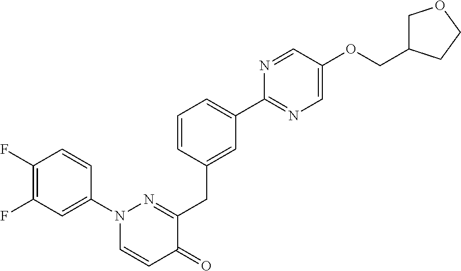 | rac-1-(3,4- difluorophenyl)- 3-{3-[5- (tetrahydrofuran-3- ylmethoxy)pyrimidin-2- yl]benzyl}pyridazin- 4(1H)-one | Calc'd 477, found 477 |
| 329 | 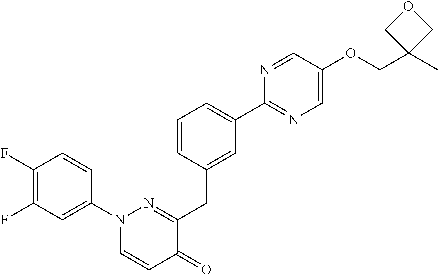 | 1-(3,4- difluorophenyl)- 3-(3-{5-[(3- methyloxetan-3- yl)methoxy]pyrimidin-2- yl}benzyl)pyridazin- 4(1H)-one | Calc'd 477, found 477 |
| 330 | 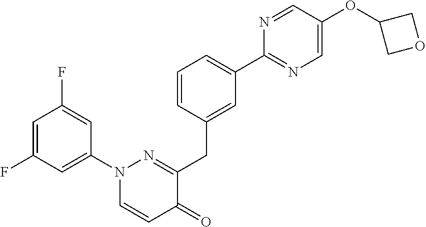 | 1-3,5- difluorophenyl)- 3-{3-[5- (oxetan-3- yloxy)pyrimidin-2- yl]benzyl}pyridazin- 4(1H)-one | Calc'd 449, found 449 |
| 331 | 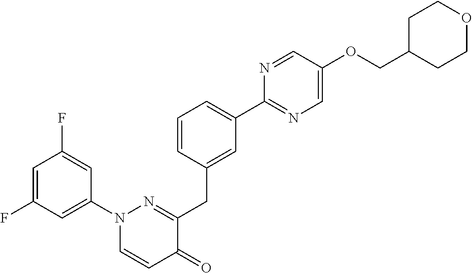 | 1-(3,5- difluorophenyl)- 3-{3-[5- (tetrahydro-2H- pyran-4- ylmethoxy)pyrimidin-2- yl]benzyl}pyridazin- 4(1H)-one | Calc'd 491, found 491 |
| 332 | 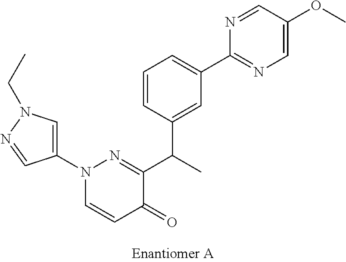 | 1-(1-ethyl-1H- pyrazol-4-yl)-3- {(1S or R)-1-[3-(5- methoxypyrimidin-2- yl)phenyl]ethyl} pyridazin- 4(1H)-one | Calc'd 403, found 403 |
| 333 | 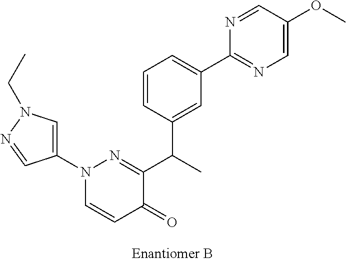 | 1-(1-ethyl-1H- pyrazol-4-yl)-3- {(1R or S)-1-[3-(5- methoxypyrimidin-2- yl)phenyl]ethyl} pyridazin- 4(1H)-one | Calc'd 403, found 403 |

The following intermediate was prepared from Intermediate #139 and Intermediate #183 according to Scheme 6 following a similar procedure described for Example #259, which can be achieved by those of ordinary skill in the art of organic synthesis.

| | | | Exact |
|---|---|---|---|
| | | | Mass |
| Intermediate | Structure | IUPAC Name | [M + H]⁺ |
| 150 | 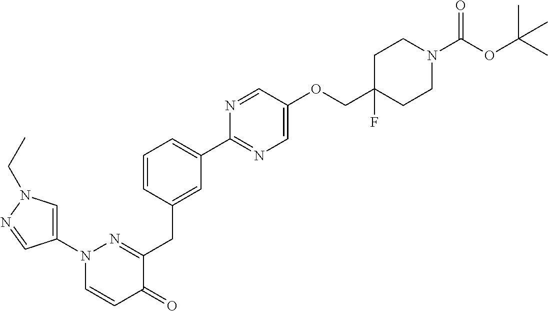 | tert-butyl 4- ({[2-(3-{[1-(1- ethyl-1H- pyrazol-4-yl)-4- oxo-1,4- dihydropyridazin- 3- yl]methyl}phen- yl)pyrimidin-5- yl]oxy}methyl)- 4- fluoropiperidine- 1-carboxylate | Calc'd 590, found 590 |

Procedures for the preparation of the alkyl halides (Intermediates #181 and 184-187) and mesylate (Intermediate #183) used in the synthesis of Examples #259, 269, 271, 273 and 290 and Intermediate #150 are shown below.

### Scheme 6

### Example #334

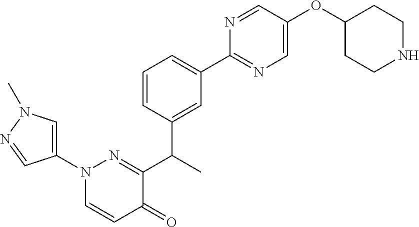

### rac-1-(1-Methyl-1H-pyrazol-4-yl)-3-(1-{3-[5-(piperidin-4-yloxy)pyrimidin-2-yl]phenyl}ethyl)pyridazin-4(1H)-one

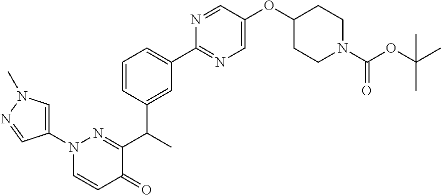

### Step 1. rac-tert-Butyl 4-{[2-(3-{1-[1-(1-methyl-1H-pyrazol-4-yl)-4-oxo-1,4-dihydropyridazin-3-yl]ethyl}phenyl)pyrimidin-5-yl]oxy}piperidine-1-carboxylate

rac-3-{1-[3-(5-Hydroxypyrimidin-2-yl)phenyl]ethyl}-1-(1-methyl-1H-pyrazol-4-yl)pyridazin-4(1H)-one (Intermediate #147, 50 mg, 0.134 mmol) and tert-butyl 4-[(methylsulfonyl)oxy]piperidine-1-carboxylate (75 mg, 0.27 mmol) were dissolved in DMF (1 mL) and cooled to 0° C. NaI (60 wt %, 8.0 mg, 0.2 mmol) was added and the reaction mixture was stirred at r.t. for 18 hours before heating to 60° C. for 24 hrs. The mixture was diluted with EtOAc and washed with saturated NaHCO₃ and brine. The organic layer was dried over Na₂SO₄, filtered and concentrated in vacuo while loading onto silica. Purification of the residue by flash chromatography (MPLC, 0-10% MeOH-EtOAc) gave rac-tert-butyl 4-{[2-(3-{1-[1-(1-methyl-1H-pyrazol-4-yl)-4-oxo-1,4-dihydropyridazin-3-yl]ethyl}phenyl)pyrimidin-5-yl]oxy}piperidine-1-carboxylate as a yellow foam.

LRMS (ESI) calc'd for C30H36N7O4 [M+H]⁺: 558. Found: 558.

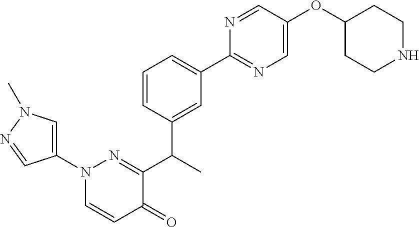

### Step 2. rac-1-(1-Methyl-1H-pyrazol-4-yl)-3-(1-{3-[5-(piperidin-4-yloxy)pyrimidin-2-yl]phenyl}ethyl)pyridazin-4(1H)-one

rac-tert-Butyl 4-{[2-(3-{1-[1-(1-methyl-1H-pyrazol-4-yl)-4-oxo-1,4-dihydropyridazin-3-yl]ethyl}phenyl)pyrimidin-5-yl]oxy}piperidine-1-carboxylate (40 mg, 0.07 mmol) was dissolved in DCM (0.5 mL) and TFA (0.5 mL) was added. The reaction mixture was stirred at r.t. for 30 min. The solvent was removed in vacuo and purified by mass-triggered reverse phase preparative HPLC. Fractions containing the pure compound were collected and the free base was liberated using PL-HCO₃ cartridges (Stratospheres™, 0.9 mmol). The filtrate was concentrated in vacuo to afford rac-1-(1-methyl-1H-pyrazol-4-yl)-3-(1-{3-[5-(piperidin-4-yloxy)pyrimidin-2-yl]phenyl}ethyl)pyridazin-4(1H)-one as a colorless foam.

LRMS (ESI) calc'd for C25H28N7O2 [M+H]⁺: 457. Found: 457.

The following examples were prepared from Examples #288-292 and Intermediate #150 according to Scheme 6 following a similar procedure described for Example 336 Step 2, which can be achieved by those of ordinary skill in the art of organic synthesis.

| | | | Exact |
|---|---|---|---|
| | | IUPAC | Mass |
| Example | Structure | Name | [M + H]⁺ |
| 335 | 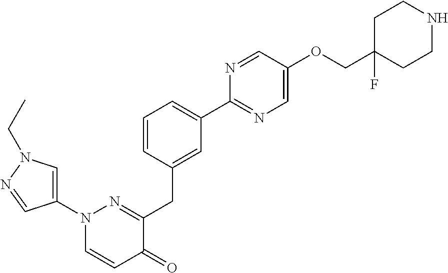 | 1-(1-ethyl-1H- pyrazol-4-yl)- 3-(3-{5-[(4- fluoropiperidin- 4- yl)methoxy] pyrimidin-2- yl}benzyl)pyri- dazin-4(1H)- one | Calc'd 490, found 490 |
| 336 | 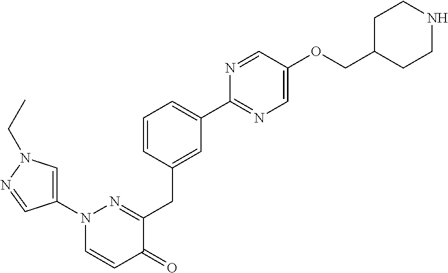 | 1-(1-ethyl-1H- pyrazol-4-yl)- 3-{3-[5- (piperidin-4- ylmethoxy) pyrimidin-2- yl]benzyl}pyri- dazin-4(1H)- one | Calc'd 472, found 472 |
| 337 | 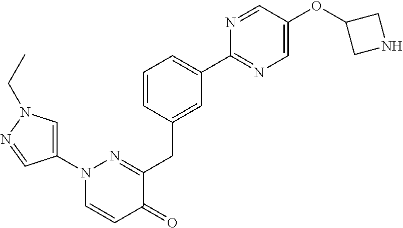 | 3-{3-[5- (azetidin-3- yloxy)pyrimi- din-2- yl]benzyl}-1- (1-ethyl-1H- pyrazol-4- yl)pyridazin- 4(1H)-one | Calc'd 430, found 430 |
| 338 | 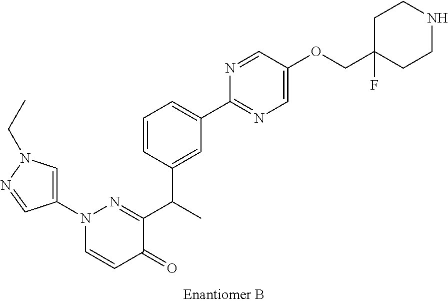 | 1-(1-ethyl-1H- pyrazol-4-yl)- 3-[(1R or S)- 1-(3-{5-[(4- fluoropiperidin- 4- yl)methoxy] pyrimidin-2- yl}phenyl)eth- yl]pyridazin- 4(1H)-one | Calc'd 504, found 504 |
| 339 | 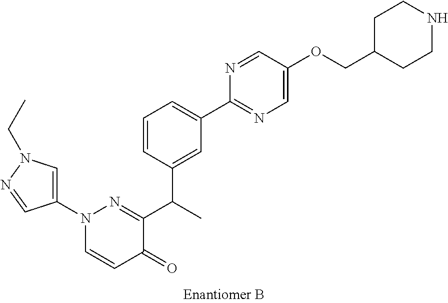 | 1-(1-ethyl-1H- pyrazol-4-yl)- 3-((1R or S)- 1-{3-[5- (piperidin-4- ylmethoxy) pyrimidin-2- yl]phenyl}eth- yl)pyridazin- 4(1H)-one | Calc'd 486, found 486 |
| 340 | 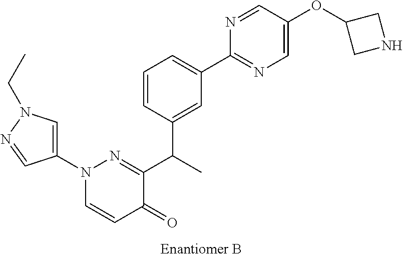 | 3-((1R or S)- 1-{3-[5- (azetidin-3- yloxy)pyrimi- din-2- yl]phenyl}eth- yl)-1-(1-ethyl- 1H-pyrazol-4- yl)pyridazin- 4(1H)-one | Calc'd 444, found 444 |

### Scheme 6

### Example #341

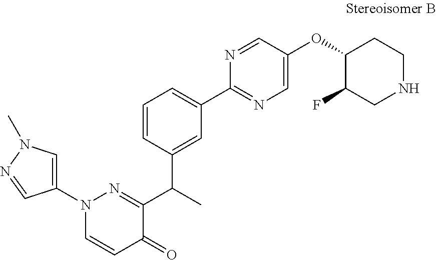

### 3-{(1R or S)-1-[3-(5-{[(3R or S,4R or S)-3-Fluoropiperidin-4-yl]oxy}pyrimidin-2-yl)phenyl]ethyl}-1-(1-methyl-1H-pyrazol-4-yl)pyridazin-4(1H)-one (Stereoisomer B)

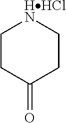

### Step 1. Piperidin-4-one hydrochloride

To a solution of tert-butyl 4-oxopiperidine-1-carboxylate (2300 g, 11.6 mol) in 1,4-dioxane was added a solution of HCl (g)/1,4-dioxane (4 L, 10 mol/L) slowly at 0° C. After the addition, the reaction mixture was stirred for 4 h and TLC (EtOAc/petroleum ether=1:5) showed the reaction was complete. The solvent was removed in vacuo to afford piperidin-4-one hydrochloride as a brown solid.

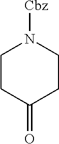

### Step 2. Benzyl 4-oxopiperidine-1-carboxylate

To a stirred solution of piperidin-4-one hydrochloride (1567 g, 11.6 mol) and triethylamine (1400 g, 13.87 mol) in dichloromethane (12 L) was added benzyl chloroformate (1965 g, 11.55 mol) dropwise at 0° C. After the addition, the reaction mixture was allowed to warm to room temperature and stirred overnight. TLC (EtOAc/petroleum ether=1:5) showed the reaction was complete. The mixture was washed with water (3 L) and brine (1 L), dried over sodium sulfate and concentrated in vacuo to give benzyl 4-oxopiperidine-1-carboxylate as a colorless oil.

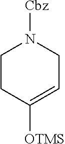

### Step 3. Benzyl 4-[(trimethylsilyl)oxy]-3,6-dihydropyridine-1(2H)-carboxylate

To a stirred solution of benzyl 4-oxopiperidine-1-carboxylate (300 g, 1.29 mol) and TMSCl (280 g, 2.58 mol) in DMF (1600 mL) was added triethylamine (457 g, 4.52 mol) at room temperature. After the addition, the reaction mixture was slowly heated to 85° C. and stirred overnight. TLC (EtOAc/Petroleum ether=1:10) showed that the starting material was consumed completely. The reaction mixture was poured into aqueous NaHCO₃ (5% w/v) at 0° C. with vigorous stirring and extracted with petroleum ether (3 L, 1 L). The organic layer was dried over sodium sulfate and concentrated to afford benzyl 4-[(trimethylsilyl)oxy]-3,6-dihydropyridine-1(2H)-carboxylate as a brown oil.

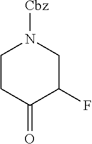

### Step 4. Benzyl 3-fluoro-4-oxopiperidine-1-carboxylate

To a solution of benzyl 4-[(trimethylsilyl)oxy]-3,6-dihydropyridine-1(2H)-carboxylate (800 g, 2.61 mol) in acetonitrile was added Selectfluor™ (932.8 g, 2.63 mol) in small portions under nitrogen at 0° C. After the addition, the reaction mixture was naturally warmed to room temperature and stirred overnight. TLC (EtOAc/petroleum ether=1:1) indicated the reaction was complete, and the solvent was removed in vacuo. The residue was triturated with ethyl acetate (5 L, 3 L) for 30 min and filtered. The filtrate was concentrated and the syrup was purified by column chromatography (EtOAc/petroleum ether=1:10 to 2:3) to afford benzyl 3-fluoro-4-oxopiperidine-1-carboxylate.

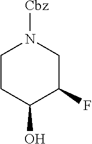

### Step 5. rac-Benzyl cis-3-fluoro-4-hydroxypiperidine-1-carboxylate

To a solution of benzyl 3-fluoro-4-oxopiperidine-1-carboxylate (200 g, 0.797 mol) in methanol (1 L) was added NaBH₄ (24.4 g, 0.636 mol) in portions at 0° C. After the addition, the mixture was stirred for 4 h and then quenched with water (200 mL). The mixture was concentrated, and then ethyl acetate and water were added to the residue. The organic layer was separated, dried over sodium sulfate and concentrated. The residue was purified by flash chromatography (EtOAc/petroleum ether=1:6) to give rac-benzyl cis-3-fluoro-4-hydroxypiperidine-1-carboxylate.

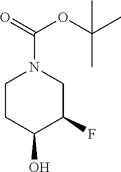

### Step 6. rac-tert-Butyl cis-3-fluoro-4-hydroxypiperidine-1-carboxylate

A mixture of rac-benzyl cis-3-fluoro-4-hydroxypiperidine-1-carboxylate (92 g, 0.363 mol), Boc₂O (83.2 g, 0.382 mol) and methanol (1 L) was stirred in the presence of Pd(OH)₂/C under hydrogen (35 psi) at room temperature. The reaction was monitored by TLC (EtOAc/petroleum ether=1:2). Upon completion, the mixture was filtered and the filtrate was concentrated in vacuo to give a white solid, which was re-crystallized from petroleum ether/methanol to afford rac-tert-butyl cis-3-fluoro-4-hydroxypiperidine-1-carboxylate as a white solid.

LRMS (ESI) calc'd for C10H19FNO3 [M+H]⁺: 219. Found: 219.

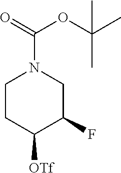

### Step 7. rac-tert-Butyl cis-3-fluoro-4-{[(trifluoromethyl)sulfonyl]oxy}piperidine-1-carboxylate

rac-tert-Butyl cis-3-fluoro-4-hydroxypiperidine-1-carboxylate (500 mg, 2.28 mmol) was dissolved in DCM (10 mL) and pyridine (0.46 mL, 5.70 mmol) was added. The reaction mixture was cooled to −20° C. and trifluoromethanesulfonic anhydride (0.58 mL, 3.42 mmol) was added. The reaction mixture was stirred for 30 min. The reaction mixture was diluted with DCM and washed with sat. NaHCO₃. The organic layer was dried over Na₂SO₄, filtered and concentrated in vacuo while loading onto silica. Purification of the residue by flash chromatography (MPLC, 2-20% EtOAc-hexane) gave rac-tert-butyl cis-3-fluoro-4-{[(trifluoromethyl)sulfonyl]oxy}piperidine-1-carboxylate as a white gum.

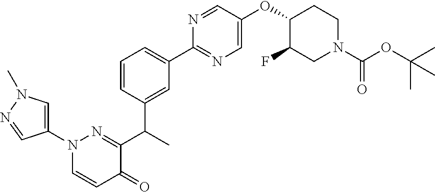

### Step 8. tert-Butyl trans-3-fluoro-4-[2-(3-{1-{1-(1-methyl-1H-pyrazol-4-yl)-4-oxo-1,4-dihydropyridazin-3-yl]ethyl}phenyl)pyrimidin-5-yl]oxy}piperidine-1-carboxylate (mixture of 4 stereoisomers)

rac-3-{1-[3-(5-Hydroxypyrimidin-2-yl)phenyl]ethyl}-1-(1-methyl-1H-pyrazol-4-yl)pyridazin-4(1H)-one (Intermediate #147, 88 mg, 0.235 mmol) and rac-tert-butyl cis-3-fluoro-4-{[(trifluoromethyl)sulfonyl]oxy}piperidine-1-carboxylate (140 mg, 0.470 mmol) were dissolved in DMF (1 mL) and cooled to 0° C. Sodium hydride (60 wt %, 14 mg, 0.35 mmol) was then added and the red reaction mixture warmed to r.t and stirred for 18 hours. The reaction mixture was diluted with EtOAc then washed with saturated NaHCO₃ and brine. The organic layer was dried over Na₂SO₄, filtered and concentrated in vacuo while loading onto silica. Purification of the residue by flash chromatography (MPLC, 0-10% MeOH-EtOAc) gave tert-butyl trans-3-fluoro-4-{[2-(3-{1-[1-(1-methyl-1H-pyrazol-4-yl)-4-oxo-1,4-dihydropyridazin-3-yl]ethyl}phenyl)pyrimidin-5-yl]oxy}piperidine-1-carboxylate (mixture of 4 stereoisomers) as a yellow foam.

LRMS (ESI) calc'd for C30H35FN7O4 [M+H]⁺: 576. Found: 576.

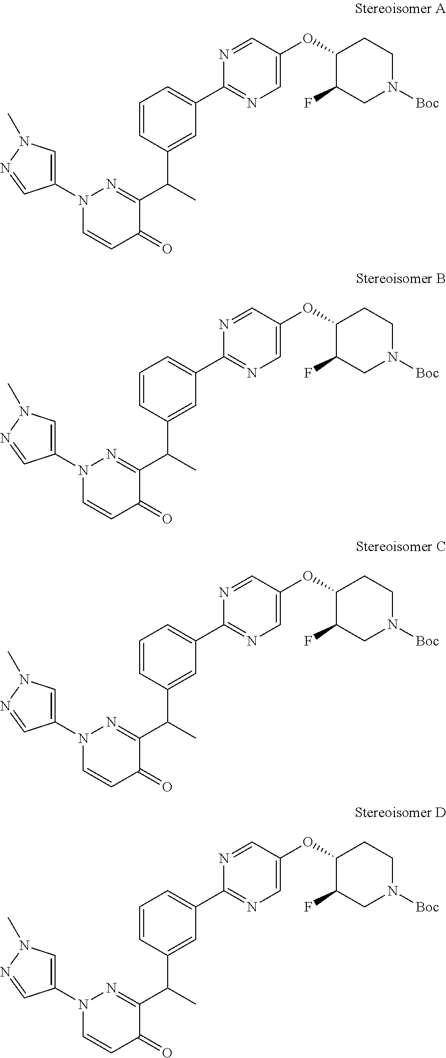

### Step 9. tert-Butyl (3R or S,4R or S)-3-fluoro-4-{[2-(3-{(1R or S)-1-[1-(1-methyl-1H-pyrazol-4-yl)-4-oxo-1,4-dihydropyridazin-3-yl]ethyl}phenyl)pyrimidin-5-yl]oxy}piperidine-1-carboxylate (Stereoisomer A), tert-butyl (3R or S,4R or S)-3-fluoro-4-{[2-(3-{(1R or S)-1-[1-(1-methyl-1H-pyrazol-4-yl)-4-oxo-1,4-dihydropyridazin-3-yl]ethyl}phenyl)pyrimidin-5-yl]oxy}piperidine-1-carboxylate (Stereoisomer B), tert-butyl (3R or S,4R or S)-3-fluoro-4-{[2-(3-{(1R or S)-1-[1-(1-methyl-1H-pyrazol-4-yl)-4-oxo-1,4-dihydropyridazin-3-yl]ethyl}phenyl)pyrimidin-5-yl]oxy}piperidine-1-carboxylate (Stereoisomer C) and tert-butyl (3R or S,4R or S)-3-fluoro-4-{[2-(3-{(1R or S)-1-[1-(1-methyl-1H-pyrazol-4-yl)-4-oxo-1,4-dihydropyridazin-3-yl]ethyl}phenyl)pyrimidin-5-yl]oxy}piperidine-1-carboxylate (Stereoisomer D)

The four stereoisomers of tert-butyl trans-3-fluoro-4-{[2-(3-{1-[1-(1-methyl-1H-pyrazol-4-yl)-4-oxo-1,4-dihydropyridazin-3-yl]ethyl}phenyl)pyrimidin-5-yl]oxy}piperidine-1-carboxylate (119 mg, 0.393 mmol) were separated by SFC (Berger Multigram II SFC, column: Chiral Technology OJ-H 2.1×25 cm, 5 μM, mobile phase: 20% MeOH/80% CO_{2(I)}, flow rate: 70 mL/min, 11 min run time) to give tert-butyl (3R or S,4R or S)-3-fluoro-4-{[2-(3-{(1R or S)-1-[1-(1-methyl-1H-pyrazol-4-yl)-4-oxo-1,4-dihydropyridazin-3-yl]ethyl}phenyl)pyrimidin-5-yl]oxy}piperidine-1-carboxylate (Stereoisomer A), tert-butyl (3R or S,4R or S)-3-fluoro-4-{[2-(3-{(1R or S)-1-[1-(1-methyl-1H-pyrazol-4-yl)-4-oxo-1,4-dihydropyridazin-3-yl]ethyl}phenyl)pyrimidin-5-yl]oxy}piperidine-1-carboxylate (Stereoisomer B), tert-butyl (3R or S,4R or S)-3-fluoro-4-{[2-(3-{(1R or S)-1-[1-(1-methyl-1H-pyrazol-4-yl)-4-oxo-1,4-dihydropyridazin-3-yl]ethyl}phenyl)pyrimidin-5-yl]oxy}piperidine-1-carboxylate (Stereoisomer C) and tert-butyl (3R or S,4R or S)-3-fluoro-4-{[2-(3-{(1R or S)-1-[1-(1-methyl-1H-pyrazol-4-yl)-4-oxo-1,4-dihydropyridazin-3-yl]ethyl}phenyl)pyrimidin-5-yl]oxy}piperidine-1-carboxylate (Stereoisomer D).

Stereoisomer A: LRMS (ESI) calc'd for C30H35FN7O4 [M+H]⁺: 576. Found: 576.

Stereoisomer B: LRMS (ESI) calc'd for C30H35FN7O4 [M+H]⁺: 576. Found: 576.

Stereoisomer C: LRMS (ESI)-calc'd for C30H35FN7O4 [M+H]⁺: 576. Found: 576.

Stereoisomer D: LRMS (ESI) calc'd for C30H35FN7O4 [M+H]⁺: 576. Found: 576.

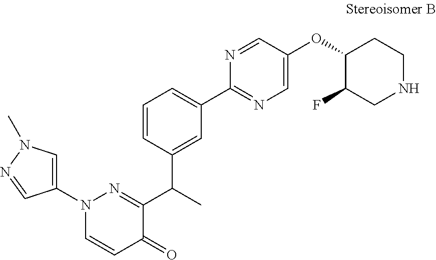

### Step 10. 3-{(1R or S)-1-[3-(5-{[3R or S,4R or S)-3-Fluoropiperidin-4-yl]oxy}pyrimidin-2-yl)phenyl]ethyl}-1-(1-methyl-1H-pyrazol-4-yl)pyridazin-4(1H-one (Stereoisomer B)

3-{(1R or S)-1-[3-(5-{[(3R or S,4R or S)-3-Fluoropiperidin-4-yl]oxy}pyrimidin-2-yl)phenyl]ethyl}-1-(1-methyl-1H-pyrazol-4-yl)pyridazin-4(1H)-one (Stereoisomer B) was prepared from tert-butyl (3R or S,4R or S)-3-fluoro-4-{[2-(3-{(R or S)-1-[1-(1-methyl-1H-pyrazol-4-yl)-4-oxo-1,4-dihydropyridazin-3-yl]ethyl}phenyl)pyrimidin-5-yl]oxy}piperidine-1-carboxylate (Stereoisomer B) according to the procedure described for rac-1-(1-methyl-1H-pyrazol-4-yl)-3-(1-{3-[5-(piperidin-4-yloxy)pyrimidin-2-yl]phenyl}ethyl)pyridazin-4(1H)-one (Example #334 Step 2).

LRMS (ESI) calc'd for C25H27FN7O4 [M+H]⁺: 476. Found: 476.

The following examples were prepared from Example #341 Step 9 Stereoisomers C and D according to Scheme 6 following a similar procedure described for Example #334 Step 2, which can be achieved by those of ordinary skill in the art of organic synthesis.

| | | | Exact |
|---|---|---|---|
| | | IUPAC | Mass |
| Example | Structure | Name | [M + H]⁺ |
| 342 | 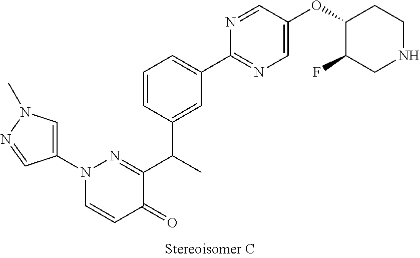 | 3-{(1R or S)- 1-[3-(5-{[(3R or S, 4R or S)- 3- fluoropiperidin- 4- yl]oxy}pyrimi- din-2- yl)phenyl]ethyl}- 1-(1- methyl-1H- pyrazol-4- yl)pyridazin- 4(1H)-one | Calc'd 476, found 476 |
| 343 | 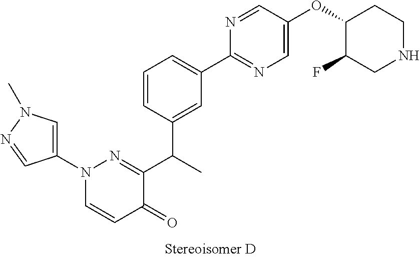 | 3-{(1R or S)- 1-[3-(5-{[(3R or S, 4R or S)- 3- fluoropiperidin- 4- yl]oxy}pyrimi- din-2- yl)phenyl]ethyl}- 1-(1- methyl-1H- pyrazol-4- yl)pyridazin- 4(1H)-one | Calc'd 476, found 476 |

### Scheme 6

### Example #344

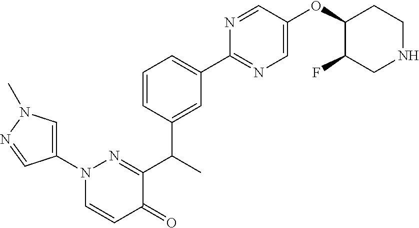

### 3-[1-(3-{5-[cis-3-Fluoropiperidin-4-yl)oxy]pyrimidin-2-yl}phenyl)ethyl]-1-(1-methyl-1H-pyrazol-4-yl)pyridazin-4(1H)-one (mixture of 4 stereoisomers)

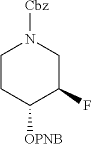

### Step 1. rac-Benzyl trans-3-fluoro-4-[(4-nitrobenzyl)oxy]piperidine-1-carboxylate

To a solution of triphenylphosphine. (120.1 g, 0.458 mol) in THF (2 L) was added DIAD (92.6 g, 0.458 mol) with stirring under nitrogen at 0° C. The resulting suspension was stirred for 40 min and then a solution of rac-benzyl cis-3-fluoro-4-hydroxypiperidine-1-carboxylate (Example #341 Step 5, 58g, 0.229 mol) and 4-nitrobenzoic acid (45.9 g, 0.275 mol) in THF was added slowly over 1.5 h. The resulting orange solution was allowed to warm to room temperature and stirred for about 48 h. The mixture was concentrated under reduced pressure, and the residual oil was purified by column chromatography (EtOAc:petroleum ether=1:40) to afford rac-benzyl trans-3-fluoro-4-[(4-nitrobenzyl)oxy]piperidine-1-carboxylate as an off-white solid.

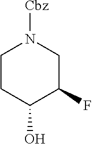

### Step 2. rac-Benzyl trans-3-fluoro-4-hydroxypiperidine-1-carboxylate

To a solution of rac-benzyl trans-3-fluoro-4-[(4-nitrobenzyl)oxy]piperidine-1-carboxylate (180 g, 0.45 mol) in THF/H₂O (1500 mL) was added LiOH (39 g, 0.9 mol) in portions at 0° C. After the addition, the reaction mixture was allowed to warm to room temperature and stirred overnight. TLC (EtOAc:petroleum ether=1:2) showed the starting material was consumed completely. The reaction mixture was extracted with EtOAc (500 mL x3). The combined organic layers were washed with brine (800 mL), dried over anhydrous Na₂SO₄ and purified by flash chromatography (EtOAc:petroleum ether=1:20) to afford rac-benzyl trans-3-fluoro-4-hydroxypiperidine-1-carboxylate as a white solid.

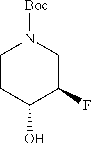

### Step 3. rac-tert-Butyl trans-3-fluoro-4-hydroxypiperidine-1-carboxylate

rac-tert-Butyl trans-3-fluoro-4-hydroxypiperidine-1-carboxylate was prepared from rac-benzyl trans-3-fluoro-4-hydroxypiperidine-1-carboxylate according to the procedure described for rac-tert-butyl cis-3-fluoro-4-hydroxypiperidine-1-carboxylate (Example #341 Step 6).

LRMS (ESI) calc'd for C10H19FNO3 [M+H]⁺: 219. Found: 219.

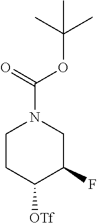

### Step 4. rac-tert-Butyl trans-3-fluoro-4-{[(trifluoromethyl)sulfonyl]oxy}piperidine-1-carboxylate

rac-tert-Butyl trans-3-fluoro-4-{[(trifluoromethyl)sulfonyl]oxy}piperidine-1-carboxylate was prepared from rac-tert-butyl trans-3-fluoro-4-hydroxypiperidine-1-carboxylate according to the procedure described for rac-tert-butyl cis-3-fluoro-4-{[(trifluoromethyl)sulfonyl]oxy}piperidine-1-carboxylate (Example #341 Step 7).

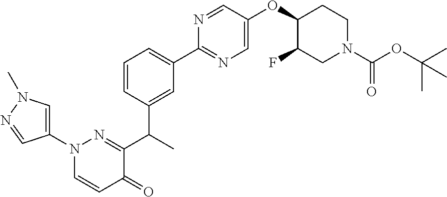

### Step 5. tert-Butyl cis-3-fluoro-4-{[2-(3-{1-[1-(1-methyl-1H-pyrazol-4-yl)-4-oxo-1,4-dihydropyridazin-3-yl]ethyl}phenyl)pyrimidin-5-yl]oxy}piperidine-1-carboxylate (mixture of 4 stereoisomers)

tert-Butyl cis-3-fluoro-4-{[2-(3-{1-[1-(1-methyl-1H-pyrazol-4-yl)-4-oxo-1,4-dihydropyridazin-3-yl]ethyl}phenyl)pyrimidin-5-yl]oxy}piperidine-1-carboxylate (mixture of 4 stereoisomers) was prepared from rac-3-({1-[3-(5-hydroxypyrimidin-2-yl)phenyl]ethyl}-1-(1-methyl-1H-pyrazol-4-yl)pyridazin-4(1H)-one (Intermediate #147) and rac-tert-butyl trans-3-fluoro-4-{[(trifluoromethyl)sulfonyl]oxy}piperidine-1-carboxylate according to the procedure described for tert-butyl trans-3-fluoro-4-{[2-(3-{1-[1-(1-methyl-1H-pyrazol-4-yl)-4-oxo-1,4-dihydropyridazin-3-yl]ethyl}phenyl)pyrimidin-5-yl]oxy}piperidine-1-carboxylate (mixture of 4 stereoisomers, Example #341 Step 8).

LRMS (ESI) calc'd for C25H27FN7O4 [M+H]⁺: 476. Found: 476.

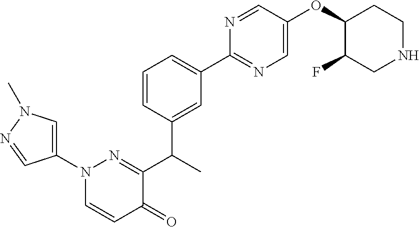

### Step 6. 3-[1-(3-{5-[(cis-3-Fluoropiperidin-4-yl)oxy]pyrimidin-2-yl}phenyl)ethyl]-1-(1-methyl-1H-pyrazol-4-yl)pyridazin-4(1H)-one (mixture of 4 stereoisomers)

3-[1-(3-{5-[(cis-3-Fluoropiperidin-4-yl)oxy]pyrimidin-2-yl}phenyl)ethyl]-1-(1-methyl-1H-pyrazol-4-yl)pyridazin-4(1H)-one (mixture of 4 stereoisomers) was prepared from tert-butyl cis-3-fluoro-4-{[2-(3-{1-[1-(1-methyl-1H-pyrazol-4-yl)-4-oxo-1,4-dihydropyridazin-3-yl]ethyl}phenyl)pyrimidin-5-yl]oxy}piperidine-1-carboxylate (mixture of 4 stereoisomers) according to the procedure described for rac-1-(1-methyl-1H-pyrazol-4-yl)-3-(1-{3-[5-(piperidin-4-yloxy).pyrimidin-2-yl]phenyl}ethyl)pyridazin-4(1H)-one (Example #334 Step 2):

LRMS (ESI) calc'd for C25H27FN7O4 [M+H]⁺: 476. Found: 476.

### Scheme 6

### Examples #345 and #346

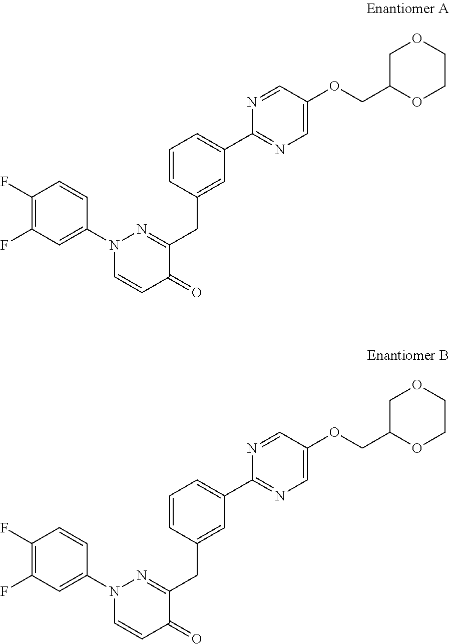

### 1-(3,4-Difluorophenyl)-3-(3-{5-[(2R or S)-1,4-dioxan-2-ylmethoxy]pyrimidin-2-yl}benzyl)pyridazin-4(1H)-one (Enantiomer A) and 1-(3,4-difluorophenyl)-3-(3-{5-[(2S or R-1,4-dioxan-2-ylmethoxy]pyrimidin-2-yl}benzyl)pyridazin-4(1H)-one (Enantiomer B)

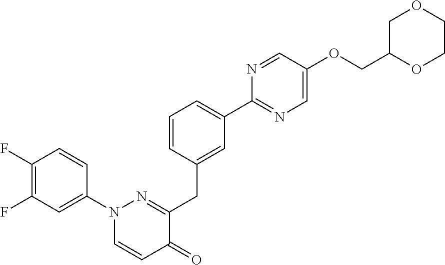

### Step 1. rac-1-(3,4-Difluorophenyl)-3-{3-[5-(1,4-dioxan-2-ylmethoxy)pyrimidin-2-yl]benzyl}pyridazin-4(1H)-one

rac-1-(3,4-Difluorophenyl)-3-{3-[5-(1,4-dioxan-2-ylmethoxy)pyrimidin-2-yl]benzyl}pyridazin-4(1H)-one was prepared from 1-(3,4-difluorophenyl)-3-[3-(5-hydroxypyrimidin-2-yl)benzyl]pyridazin-4(1H)-one (Example #188) and rac-2-(iodomethyl)-1,4-dioxane (Intermediate #182) according to the procedure described for 3-(3-{5-[2,2-difluoro-3-(morpholin-4-yl)propoxy]pyrimidin-2-yl}benzyl)-1-(1-methyl-1H-pyrazol-4-yl)pyridazin-4(1H)-one (Example #259).

LRMS (ESI) calc'd for C26H23F2N4O4 [M+H]⁺: 493. Found: 493.

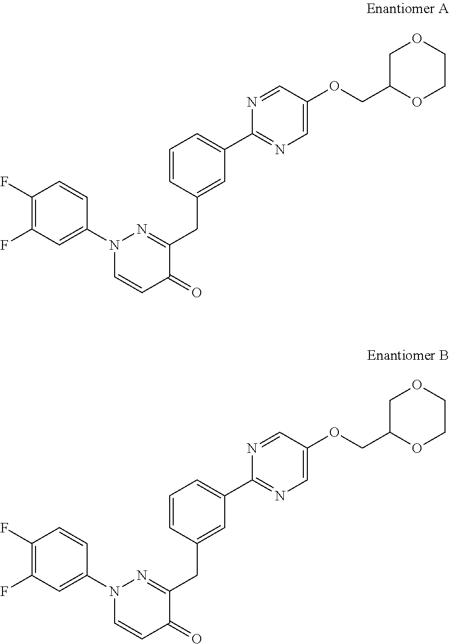

### Step 2. 1-(3,4-Difluorophenyl)-3-(3-{5-[(2R or S)-1,4-dioxan-2-ylmethoxy]pyrimidin-2-yl}benzyl)pyridazin-4(1H)-one (Enantiomer A) and 1-(3,4-difluorophenyl)-3-(3-{5-[(2S or R-1,4-dioxan-2-ylmethoxy]pyrimidin-2-yl}benzyl)pyridazin-4(1H)-one (Enantiomer B)

rac-1-(3,4-Difluorophenyl)-3-{3-[5-(1,4-dioxan-2-ylmethoxy)pyrimidin-2-yl]benzyl}pyridazin-4(1H)-one (93 mg, 0.19 mmol) was resolved by SFC (Berger Multigram II SFC, column: Chiral Technology OJ-H 2.1×25 cm, 5 μM, mobile phase: 30% methanol/70% CO_{2(I)}, flow rate: 70 mL/min, 12 min run time) to give 1-(3,4-difluorophenyl)-3-(3-{5-[(2R or S)-1,4-dioxan-2-ylmethoxy]pyrimidin-2-yl}benzyl)pyridazin-4(1H)-one (Enantiomer A, Example #345) and 1-(3,4-difluorophenyl)-3-(3-{5-[(2S or R-1,4-dioxan-2-ylmethoxy]pyrimidin-2-yl}benzyl)pyridazin-4(1H)-one (Enantiomer B, Example #346).

### Example #345

LRMS (ESI) calc'd for C26H23F2N4O4 [M+H]⁺: 493. Found: 493.

### Example #346

LRMS (ESI) calc'd for C26H23F2N4O4 [M+H]⁺: 493. Found: 493.

The following examples were prepared from Examples #187-188 according to Scheme 6 following similar procedures described for Examples #345-346, which can be achieved by those of ordinary skill in the art of organic synthesis.

| | | | Exact |
|---|---|---|---|
| | | | Mass |
| Example | Structure | IUPAC Name | [M + H]⁺ |
| 347 | 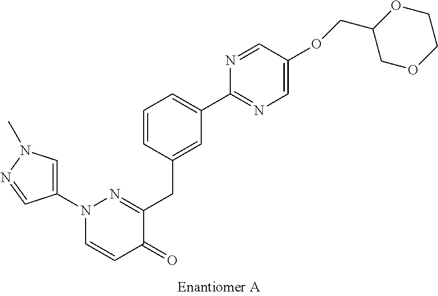 | 3-(3-{5-[(2R or- S)-1,4-dioxan- 2- ylmethoxy] pyrimidin-2- yl}benzyl)-1-(1- methyl-1H- pyrazol-4- yl)pyridazin- 4(1H)-one | Calc'd 461, found 461 |
| 348 | 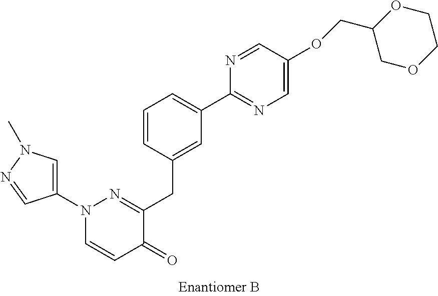 | 3-(3-{5-[(2S or R)-1,4-dioxan- 2- ylmethoxy] pyrimidin-2- yl}benzyl)-1-(1- methyl-1H- pyrazol-4- yl)pyridazin- 4(1H)-one | Calc'd 461, found 461 |
| 349 | 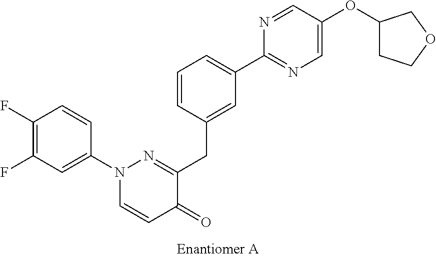 | 1-(3,4- difluorophenyl)- 3-(3-{5-[(3R or S)- tetrahydrofuran- 3- yloxy]pyrimidin- 2- yl}benzyl)pyridazin- 4(1H)-one | Calc'd 463, found 463 |
| 350 | 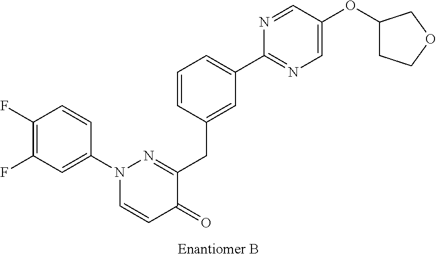 | 1-(3,4- difluorophenyl)- 3-(3-{5-[(3S or R)- tetrahydrofuran- 3- yloxy]pyrimidin- 2- yl}benzyl)pyridazin- 4(1H)-one | Calc'd 463, found 463 |
| 351 | 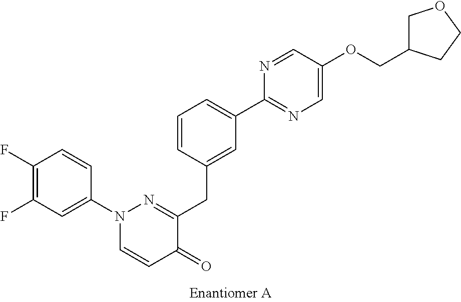 | 1-(3,4- difluorophenyl)- 3-(3-{5-[(3R or S)- tetrahydrofuran- 3- ylmethoxy] pyrimidin-2- yl}benzyl)pyridazin- 4(1H)-one | Calc'd 477, found 477 |
| 352 | 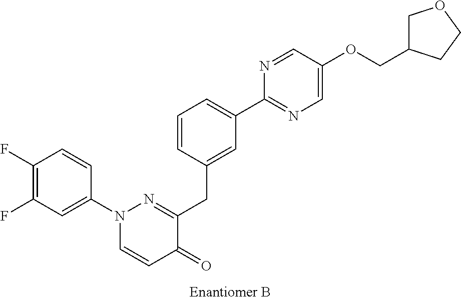 | 1-(3,4- difluorophenyl)- 3-(3-{5-[(3S or R)- tetrahydrofuran- 3- ylmethoxy] pyrimidin-2- yl}benzyl)pyridazin- 4(1H)-one | Calc'd 477, found 477 |

Procedures for the preparation of rac-2-(iodomethyl)-1,4-dioxane (Intermediate #182) used in the synthesis of Examples #345-348 are shown below.

### Scheme 6

### Example #353

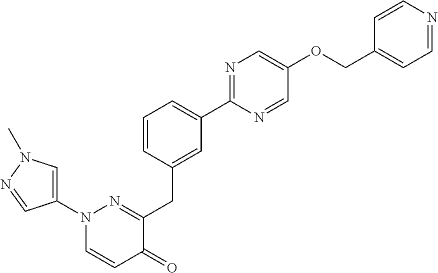

### 1-(1-Methyl-1H-pyrazol-4-yl)-3-{3-[5(pyridin-4-ylmethoxy)pyrimidin-2-yl]benzyl}pyridazin-4(1H)-one

### Step 1. 1-(1-Methyl-1H-pyrazol-4-yl)-3-{3-[5-(pyridin-4-ylmethoxy)pyrimidin-2-yl]benzyl}pyridazin-4(1H)-one

To a solution of 3-[3-(5-hydroxypyrimidin-2-yl)benzyl]-1-(1-methyl-1H-pyrazol-4-yl)pyridazin-4(1H)-one (Example #187, 50 mg, 0.14 mmol) in DMSO (2 mL) was added 4-(bromomethyl)pyridine hydrobromide (42 mg, 0.166 mmol), cesium carbonate (181 mg, 0.156 mmol) and tetrabutylammoniumiodide (5 mg, 0.014 mmol). The reaction was stirred at r.t. for 18 hours. The reaction was filtered and purified by reverse phase preparative HPLC (0-80% MeCN—H₂O, 0.05% TFA) and the free base liberated using PL-HCO₃ cartridges (Stratospheres™, 0.9 mmol) to afford 1-(1-methyl-1H-pyrazol-4-yl)-3-{3-[5-(pyridin-4-ylmethoxy)pyrimidin-2-yl]benzyl}pyridazin-4(1H)-one.

LRMS (ESI) calc'd for C25H22N7O2 [M+H]⁺: 452. Found: 452.

The following examples were prepared according to the Scheme 6 following a similar procedure described for Example #353, which can be achieved by those of ordinary skill in the art of organic synthesis.

| | | | Exact |
|---|---|---|---|
| | | | Mass |
| Example | Structure | IUPAC Name | [M + H]⁺ |
| 354 | 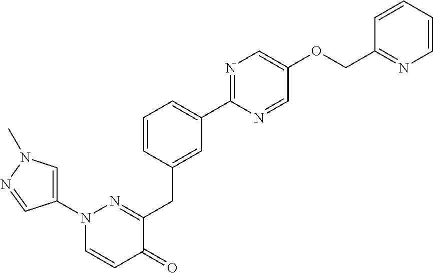 | 1-(1-methyl- 1H-pyrazol-4- yl)-3-{3-[5- (pyridin-2- ylmethoxy) pyrimidin-2- yl]benzyl}pyridazin- 4(1H)-one | Calc'd 452, found 452 |
| 355 | 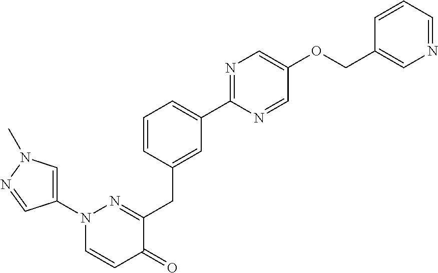 | 1-(1-methyl- 1H-pyrazol-4- yl)-3-{3-[5- (pyridin-3- ylmethoxy) pyrimidin-2- yl]benzyl}pyridazin- 4(1H)-one | Calc'd 452, found 452 |
| 356 | 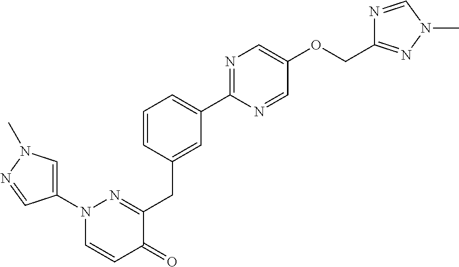 | 1-(1-methyl- 1H-pyrazol-4- yl)-3-(3-{5-[(1- methyl-1H- 1,2,4-triazol-3- yl)methoxy] pyrimidin-2- yl}benzyl)pyridazin- 4(1H)-one | Calc'd 456, found 456 |

### Scheme 6

### Example #357

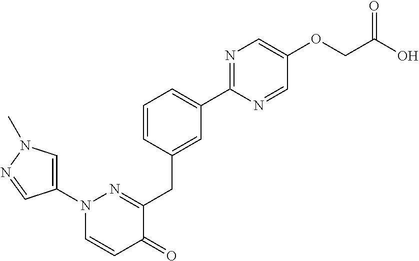

### [2-(3-{[1-(1-Methyl-1H-pyrazol-4-yl)-4-oxo-1,4-dihydropyridazin-3-yl]methyl}phenyl)pyrimidin-5-yl]oxyacetic acid

### Step 1. [2-(3-{[1-(1-Methyl-1H-pyrazol-4-yl)-4-oxo-1,4-dihydropyridazin-3-yl]methyl}phenyl)pyrimidin-5-yl]oxyacetic acid

To a solution of tert-butyl [2-(3-{[1-(1-methyl-1H-pyrazol-4-yl)-4-oxo-1,4-dihydropyridazin-3-yl]methyl}phenyl)pyrimidin-5-yl]oxyacetate (Example #287, 500 mg, 1.054 mmol) in DCM (10 mL) was added TFA (10 mL, 130 mmol). The reaction was stirred at r.t. for 18-hours. The solvent was removed in vacuo and the residue dissolved in MeOH (5 mL). The free base was liberated by eluting through a PL-HCO₃ cartridge (Stratospheres™, 0.9 mmol) and the filtrate concentrated in vacuo to give [2-(3-({[1-(1-methyl-1H-pyrazol-4-yl)-4-oxo-1,4-dihydropyridazin-3-yl]methyl}phenyl)pyrimidin-5-yl]oxyacetic acid as a white solid

LRMS (ESI) calc'd for C21H19N6O4 [M+H]⁺: 419. Found: 419.

### Scheme 6

### Example #358

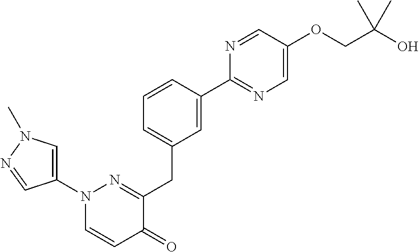

### 3-{3-[5-(2-Hydroxy-2-methylpropoxy)pyrimidin-2-yl]benzyl}-1-(1-methyl-1H-pyrazol-4-yl)pyridazin-4(1H)-one

### Step 1. 3-{3-[5-(2-Hydroxy-2-methylpropoxy)pyrimidin-2-yl]benzyl}-1-(1-methyl-1H-pyrazol-4-yl)pyridazin-4(1H-one

To a solution of 3-[3-(5-hydroxypyrimidin-2-yl)benzyl]-1-(1-methyl-1H-pyrazol-4-yl)pyridazin-4(1H)-one (Example #187, 50 mg, 0.14 mmol) in DMF (2 mL) was added K₂CO₃ (58 mg, 0.42 mmol), isobutylene oxide (62 μL, 0.69 mmol) and the reaction mixture was heated to 100° C. for 15 minutes under microwave irradiation (Biotage, Initiator). The reaction mixture was filtered and purified by reverse-phase preparative HPLC (0-80% MeCN—H₂O, 0.05% TFA). Fractions containing the pure compound were collected and the free base was liberated using PL-HCO₃ cartridges (Stratospheres™, 0.9 mmol). The filtrate was frozen and freeze dried to afford 3-{3-[5-(2-hydroxy-2-methylpropoxy)pyrimidin-2-yl]benzyl}-1-(1-methyl-1H-pyrazol-4-yl)pyridazin-4(1H)-one.

LRMS (ESI) calc'd for C23H25F3N6O3 [M+H]⁺: 433. Found: 433.

The following examples were prepared from Examples #187-189 and Intermediates #139-143 according to Scheme 6 following a similar procedure described for Example #358, which can be achieved by those of ordinary skill in the art of organic synthesis

| | | | Exact |
|---|---|---|---|
| | | | Mass |
| Example | Structure | IUPAC Name | [M + H]⁺ |
| 359 | 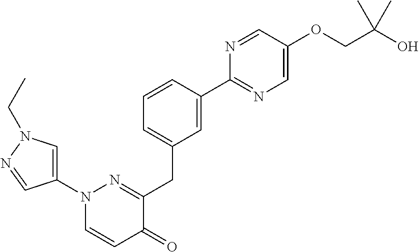 | 1-(1-ethyl-1H- pyrazol-4-yl)-3- {3-[5-(2- hydroxy-2- methylpropoxy) pyrimidin-2- yl]benzyl}pyridazin- 4(1H)-one | Calc'd 447, found 447 |
| 360 | 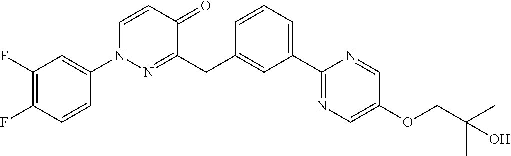 | 1-(3,4- difluorophenyl)- 3-{3-[5-(2- hydroxy-2- methylpropoxy) pyrimidin-2- yl]benzyl}pyridazin- 4(1H)-one | Calc'd 465, found 465 |
| 361 | 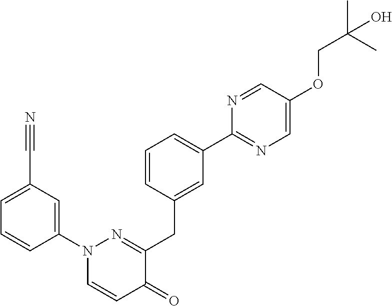 | 3-[3-{3-[5-(2- hydroxy-2- methylpropoxy) pyrimidin-2- yl]benzyl}-4- oxopyridazin- 1(4H)- yl]benzonitrile | Calc'd 454, found 454 |
| 362 | 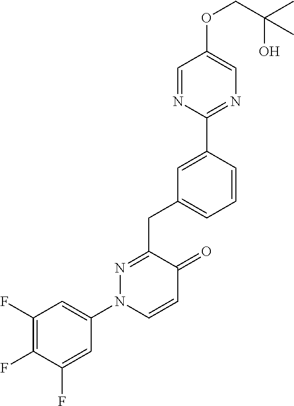 | 3-{3-[5-(2- hydroxy-2- methylpropoxy) pyrimidin-2- yl]benzyl}-1- (3,4,5- trifluorophenyl) pyridazin- 4(1H)-one | Calc'd 483, found 483 |
| 363 | 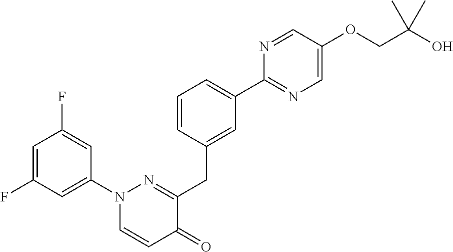 | 1-(3,5- difluorophenyl)- 3-{3-[5-(2- hydroxy-2- methylpropoxy) pyrimidin-2- yl]benzyl}pyridazin- 4(1H)-one | Calc'd 465, found 465 |
| 364 | 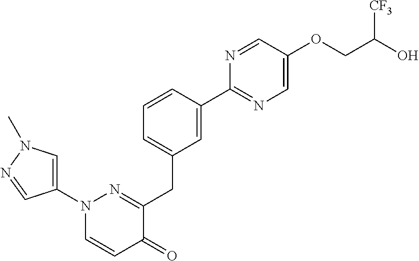 | rac-1-(1- methyl-1H- pyrazol-4-yl)-3- {3-[5-(3,3,3- trifluoro-2- hydroxypropoxy) pyrimidin-2- yl]benzyl}pyridazin- 4(1H)-one | Calc'd 473, found 473 |
| 365 | 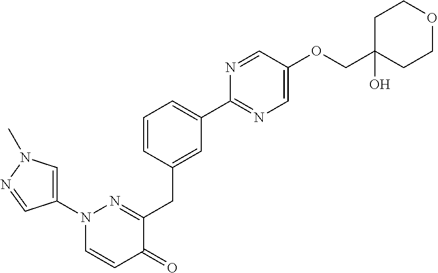 | 3-(3-{5-[(4- hydroxytetrahydro- 2H-pyran-4- yl)methoxy] pyrimidin-2- yl}benzyl)-1-(1- methyl-1H- pyrazol-4- yl)pyridazin- 4(1H)-one | Calc'd 475, found 475 |
| 366 | 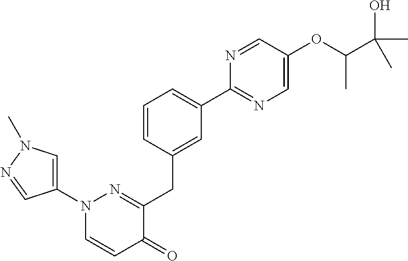 | rac-3-{3-[5-(2- hydroxy-1,2- dimethylpropoxy) pyrimidin-2- yl]benzyl}-1-(1- methyl-1H- pyrazol-4- yl)pyridazin- 4(1H)-one | Calc'd 447, found 447 |
| 367 | 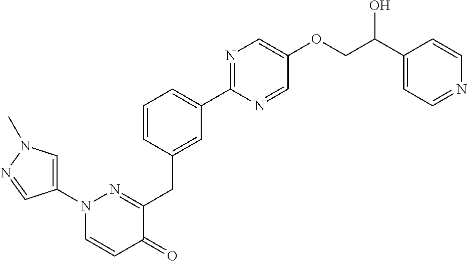 | rac-3-(3-{5-[2- hydroxy-2- (pyridin-4- yl)ethoxy] pyrimidin-2- yl}benzyl)-1-(1- methyl-1H- pyrazol-4- yl)pyridazin- 4(1H)-one | Calc'd 482, found 482 |
| 368 | 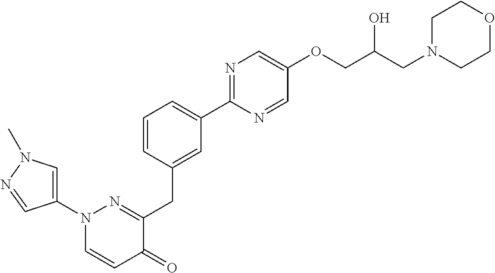 | rac-3-{3-[5-(2- hydroxy-3- morpholin-4- ylpropoxy) pyrimidin-2- yl]benzyl}-1-(1- methyl-1H- pyrazol-4- yl)pyridazin- 4(1H)-one | Calc'd 504, found 504 |
| 369 | 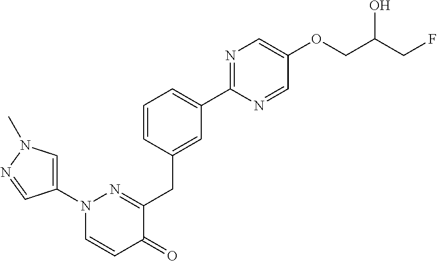 | rac-3-{3-[5-(3- fluoro-2- hydroxypropoxy) pyrimidin-2- yl]benzyl}-1-(1- methyl-1H- pyrazol-4- yl)pyridazin- 4(1H)-one | Calc'd 437, found 437 |
| 370 | 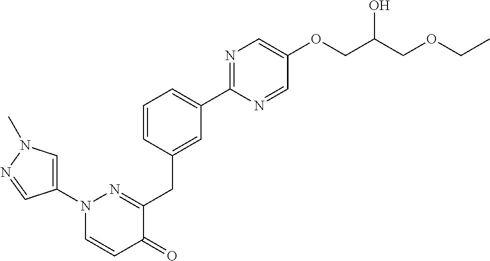 | rac-3-{3-[5-(3- ethoxy-2- hydroxypropoxy) pyrimidin-2- yl]benzyl}-1-(1- methyl-1H- pyrazol-4- yl)pyridazin- 4(1H)-one | Calc'd 463, found 463 |
| 371 | 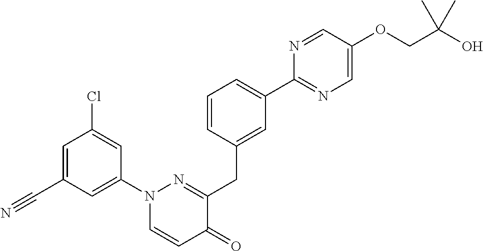 | 3-chloro-5-[3- {3-[5-(2- hydroxy-2- methylpropoxy) pyrimidin-2- yl]benzyl}-4- oxopyridazin- 1(4H)- yl]benzonitrile | Calc'd 488, found 488 |
| 372 | 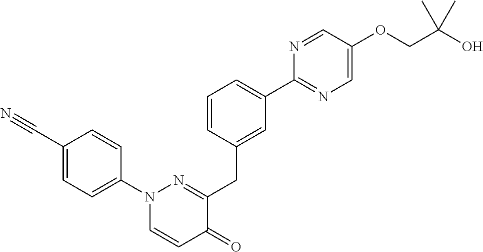 | 4-[3-{3-[5-(2- hydroxy-2- methylpropoxy) pyrimidin-2- yl]benzyl}-4- oxopyridazin- 1(4H)- yl]benzonitrile | Calc'd 454, found 454 |
| 373 | 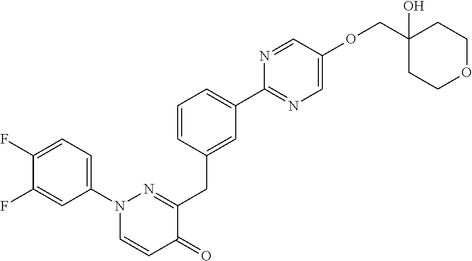 | 1-(3,4- difluorophenyl)- 3-(3-{5-[(4- hydroxytetrahydro- 2H-pyran-4- yl)methoxy] pyrimidin-2- yl}benzyl)pyridazin- 4(1H)-one | Calc'd 507, found 507 |

Procedures for the preparation of the epoxides (Intermediates #191-192) used in the synthesis of Examples #365, 367 and 373 are shown below.

### Scheme 6

### Examples #374 and #375

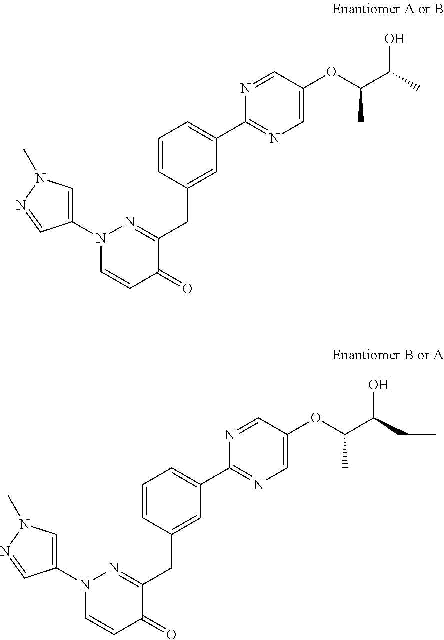

### 3-[3-(5-{[(1R,2R)-2-Hydroxy-1-methylpropyl]oxy}pyrimidin-2-yl)benzyl]-1-(1-methyl-1H-pyrazol-4-yl)pyridazin-4(1H)-one (Enantiomer A or B) and 3-[3-(5-{[(1S,2S)-2-hydroxy-1-methylpropyl]oxy}pyrimidin-2-yl)benzyl]-1-(1-methyl-1H-pyrazol-4-yl)pyridazin-4(1H)-one (Enantiomer B or A)

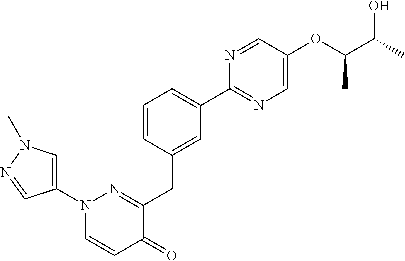

### Step 1. rac-3-{3-[5-(2-Hydroxy-1-methylpropoxy)pyrimidin-2-yl]benzyl}-1-(1-methyl-1H-pyrazol-4-yl)pyridazin-4(1H-one

To a solution of 3-[3-(5-hydroxypyrimidin-2-yl)benzyl]-1-(1-methyl-1H-pyrazol-4-yl)pyridazin-4(1H)-one (Example #187, 50 mg, 0.14 mmol) in DMF (1 mL) was added K₂CO₃ (38.4 mg, 0.277 mmol) and cis-2,3-epoxybutane (0.06 mL, 0.694 mmol). The reaction mixture was heated to 150° C. for 30 minutes under microwave irradiation (Biotage, Initiator). Additional K₂CO₃ (19 mg, 0.138 mmol) and cis-2,3-epoxybutane (0.036 mL, 0.417 mmol) were added and the reaction mixture was heated to 150° C. for 30 minutes-under microwave irradiation (Biotage, Initiator). Brine was added and the products extracted into EtOAc. The organic phase was dried over Na₂SO₄, filtered and concentrated in vacuo. Purification of the residue by flash chromatography (MPLC, 0-10% MeOH-EtOAc) gave rac-3-{3-[5-(2-hydroxy-1-methylpropoxy)pyrimidin-2-yl]benzyl}-1-(1-methyl-1H-pyrazol-4-yl)pyridazin-4(1H)-one as a white solid.

LRMS (ESI) calc'd for C23H25N6O3 [M+H]⁺: 433. Found: 433.

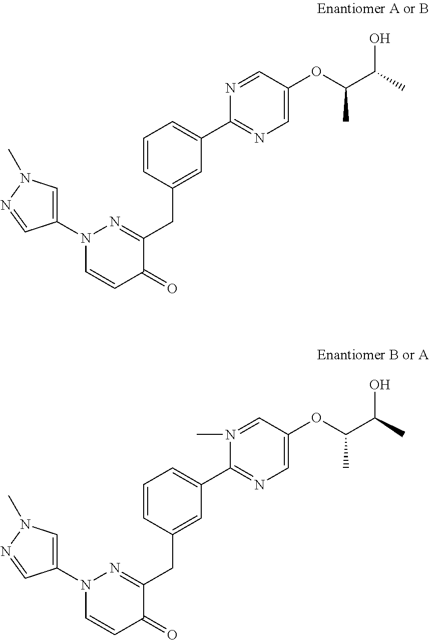

### Step 2. 3-[3-(5-{[(1R,2R)-2-Hydroxy-1-methylpropyl]oxy}pyrimidin-2-yl)benzyl]-1-(1-methyl-1H-pyrazol-4-yl)pyridazin-4(1H)-one (Enantiomer A or B) and 3-[3-(5-{[(1S,2S)-2-hydroxy-1-methylpropyl]oxy}pyrimidin-2-yl)benzyl]-1-(1-methyl-1H-pyrazol-4-ylpyridazin-4(1H-one (Enantiomer B or A)

rac-3-{3-[5-(2-Hydroxy-1-methylpropoxy)pyrimidin-2-yl]benzyl}-1-(1-methyl-1H-pyrazol-4-yl)pyridazin-4(1H)-one (20 mg, 0.046 mmol) was resolved by SFC (Berger Multigram II SFC, column: Chiral Technology AD-H 2.1×25 cm, 5 μM, mobile phase: 45% 2-propanol/55% CO₂ (1H), flow rate: 60 mL/min, 6.5 min run time) to give 3-[3-(5-{[(1R,2R)-2-hydroxy-1-methylpropyl]oxy}pyrimidin-2-yl)benzyl]-1-(1-methyl-1H-pyrazol-4-yl)pyridazin-4(1H)-one (Enantiomer A or B, Example #374) and 3-[3-(5-{[(1S,2S)-2-hydroxy-1-methylpropyl]oxy}pyrimidin-2-yl)benzyl]-1-(1-methyl-1H-pyrazol-4-yl)pyridazin-4(1H)-one (Enantiomer B or A, Example #375).

### Example #374

LRMS (ESI) calc'd for C23H25N6O3 [M+H]⁺: 433. Found: 433.

### Example #375

LRMS (ESI) calc'd for C23H25N6O3 [M+H]⁺: 433. Found: 433.

The following examples were prepared according to Scheme 6 following similar procedures described for Examples #374-375 which can be achieved by those of ordinary skill in the art of organic synthesis.

| | | | Exact |
|---|---|---|---|
| | | | Mass |
| Example | Structure | IUPAC Name | [M + H]⁺ |
| 376 | 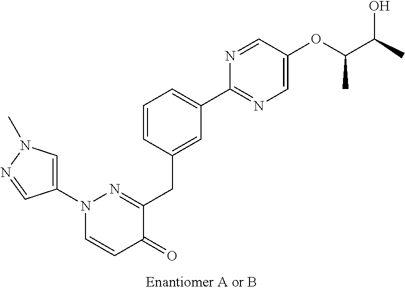 | 3-[3-(5- {[(1R,2S)-2- hydroxy-1- methylpropyl] oxy}pyrimidin-2- yl)benzyl]-1-(1- methyl-1H- pyrazol-4- yl)pyridazin- 4(1H)-one | Calc'd 433, found 433 |
| 377 | 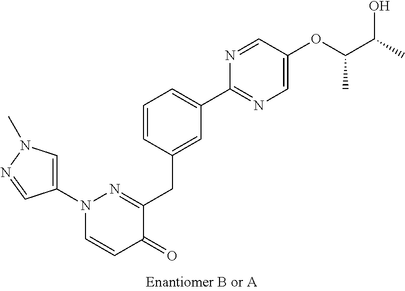 | 3-[3-(5- {[(1S,2R)-2- hydroxy-1- methylpropyl] oxy}pyrimidin-2- yl)benzyl]-1-(1- methyl-1H- pyrazol-4- yl)pyridazin- 4(1H)-one | Calc'd 433, found 433 |
| 378 | 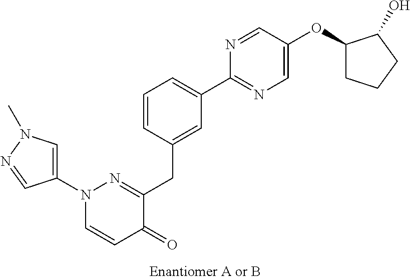 | 3-[3-(5- {[(1R,2R)-2- hydroxycyclo- pentyl]oxy}pyrimi- din-2- yl)benzyl]-1-(1- methyl-1H- pyrazol-4- yl)pyridazin- 4(1H)-one | Calc'd 445, found 445 |
| 379 | 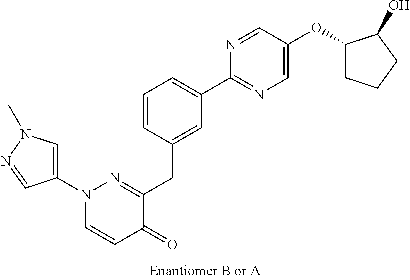 | 3-[3-(5- {[(1S,2S)-2- hydroxycyclo- pentyl]oxy}pyrimi- din-2- yl)benzyl]-1-(1- methyl-1H- pyrazol-4- yl)pyridazin- 4(1H)-one | Calc'd 445, found 445 |

### Scheme 6

### Examples #380 and 381

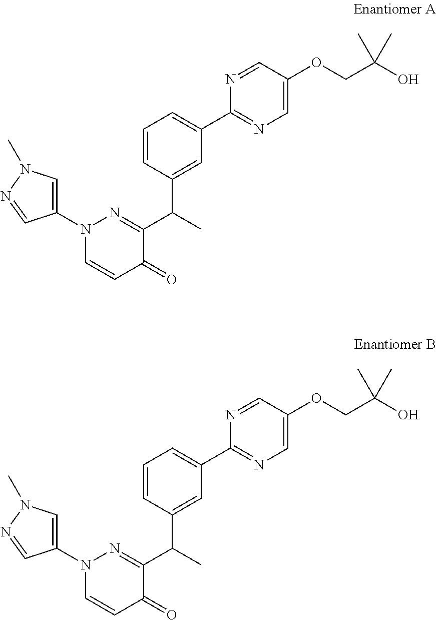

### 3-((1S or R)-1-{3-[5-(2-Hydroxy-2-methylpropoxy)pyrimidin-2-yl]phenyl}ethyl)-1-(1-methyl-1H-pyrazol-4-yl)pyridazin-4(1H)-one (Enantiomer A) and 3-((1R or S)-1-{3-[5-(2-hydroxy-2-methylpropoxy)pyrimidin-2-yl]phenyl}ethyl)-1-(1-methyl-1H-pyrazol-4-yl)pyridazin-4(1H)-one (Enantiomer B)

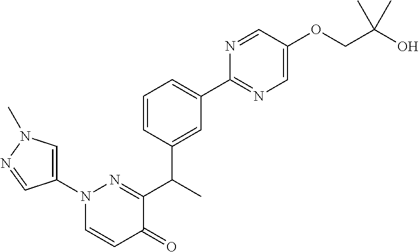

### Step 1. rac-3-1-{3-[5-(2-Hydroxy-2-methylpropoxy)pyrimidin-2-yl]-phenyl}ethyl)-1-(1-methyl-1H-pyrazol-4-yl)pyridazin-4(1H)-one

rac-3-{1-[3-(5-Hydroxypyrimidin-2-yl)phenyl]ethyl}-1-(1-methyl-1H-pyrazol-4-yl)pyridazin-4(1H)-one (Intermediate #147, 300 mg, 0.801 mmol), K₂CO₃ (221 mg, 1.603 mmol) and isobutylene oxide (0.356 mL, 4.01 mmol) were taken up in DMF (6 mL). The reaction was heated to 150° C. fox 30 minutes under microwave irradiation (Biotage; Initiator). Saturated NH₄Cl was added and the products extracted into EtOAc. The organic phase was dried over Na₂SO₄, filtered and concentrated in vacuo while loading onto silica. Purification of the residue by flash chromatography (MPLC, 0-10% MeOH-EtOAc) gave rac-3-(1-{3-[5-(2-hydroxy-2-methylpropoxy)pyrimidin-2-yl]phenyl}ethyl)-1-(1-methyl-1H-pyrazol-4-yl)pyridazin-4(1H)-one.

LRMS (ESI) calc'd for C24H27N6O3 [M+H]⁺: 447. Found: 447.

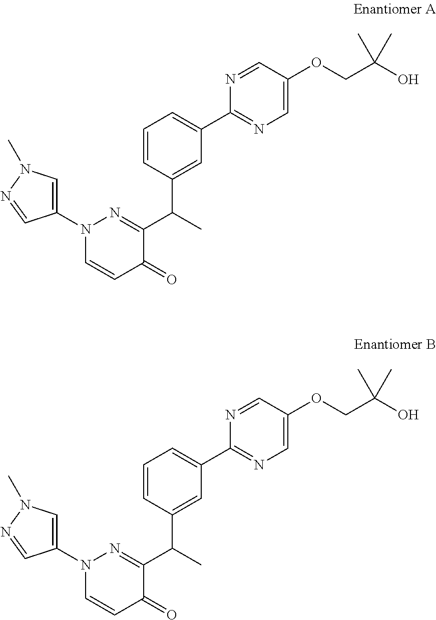

### Step 2. 3-((1S or R)-1-{3-[5-(2-Hydroxy-2-methylpropoxy)pyrimidin-2-yl]phenyl}ethyl)-1-(1-methyl-1H-pyrazol-4-yl)pyridazin-4(1H)-one (Enantiomer A) and 3-((1R or S)-1-{3-[5-(2-hydroxy-2-methylpropoxy)pyrimidin-2-yl]phenyl}ethyl)-1-(1-methyl-1H-pyrazol-4-yl)pyridazin-4(1H)-one (Enantiomer B)

rac-3-(1-{3-[5-(2-Hydroxy-2-methylpropoxy)pyrimidin-2-yl]phenyl}ethyl)-1-(1-methyl-1H-pyrazol-4-yl)pyridazin-4(1H)-one (285 mg, 0.64 mmol) was resolved by SFC (Berger Multigram II SFC, column: Chiral Technology AD-H 2.1×25 cm, 5 μM, mobile phase: 45% MeOH/55% CO₂₍ₗ₎, flow rate: 70 mL/min, 6 min run time) to give 3-((1S or R)-1-{3-[5-(2-hydroxy-2-methylpropoxy)pyrimidin-2-yl]phenyl}ethyl)-1-(1-methyl-1H-pyrazol-4-yl)pyridazin-4(1H)-one (Enantiomer A, Example #380) and 3-((1R or S)-1-{3-[5-(2-hydroxy-2-methylpropoxy)pyrimidin-2-yl]phenyl}ethyl)-1-(1-methyl-1H-pyrazol-4-yl)pyridazin-4(1H)-one (Enantiomer B, Example #381).

### Example #380

LRMS (ESI) calc'd for C24H27N6O3 [M+H]⁺: 447. Found: 447.

### Example #381

LRMS (ESI) calc'd for C24H27N6O3 [M+H]⁺: 447. Found: 447.

The following examples were prepared from Intermediate #147 and 2,2,3-trimethyloxirane according to Scheme 6 following similar procedures described for Examples #380-381 which can be achieved by those of ordinary skill in the art of organic synthesis.

| | | | Exact |
|---|---|---|---|
| | | | Mass |
| Example | Structure | IUPAC Name | [M + H]⁺ |
| 382 | 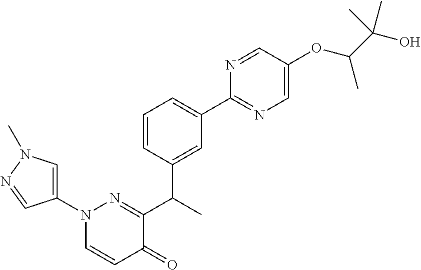 | 3-{(1R or S)-1-[3-(5- {[(1R or S)-2-hydroxy- 1,2- dimethylpropyl]oxy} pyrimidin-2- yl)phenyl]ethyl}-1-(1- methyl-1H-pyrazol-4- yl)pyridazin-4(1H)- one | Calc'd 461, found 461 |
| | Stereoisomer A | | |
| 383 | 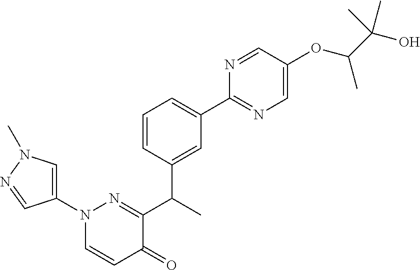 | 3-{(1R or S)-1-[3-(5- {[(1R or S)-2-hydroxy- 1,2- dimethylpropyl]oxy} pyrimidin-2- yl)phenyl]ethyl}-1-(1- methyl-1H-pyrazol-4- yl)pyridazin-4(1H)- one | Calc'd 461, found 461 |
| | Stereoisomer B | | |
| 384 | 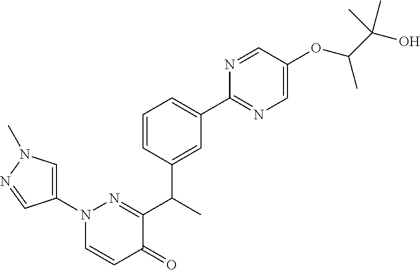 | 3-{(1R or S)-1-[3-(5- {[(1R or S)-2-hydroxy- 1,2- dimethylpropyl]oxy} pyrimidin-2- yl)phenyl]ethyl}-1-(1- methyl-1H-pyrazol-4- yl)pyridazin-4(1H)- one | Calc'd 461, found 461 |
| | Stereoisomer C | | |
| 385 | 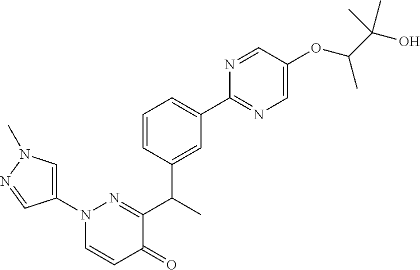 | 3-{(1R or S)-1-[3-(5- {[(1R or S)-2-hydroxy- 1,2- dimethylpropyl]oxy} pyrimidin-2- yl)phenyl]ethyl}-1-(1- methyl-1H-pyrazol-4- yl)pyridazin-4(1H)- one | Calc'd 461, found 461 |
| | Stereoisomer D | | |

### Scheme 6

### Example #386

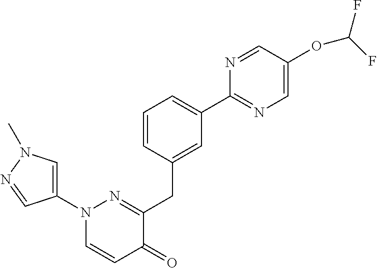

### 3-{3-[5-(Difluoromethoxy)pyrimidin-2-yl]benzyl}-1-(1-methyl-1H-pyrazol-4-yl)pyridazin-4(1H)-one

### Step 1. 3-{3-[5-(Difluoromethoxy)pyrimidin-2-yl]benzyl}-1-(1-methyl-1H-pyrazol-4-yl)pyridazin-4(1H)-one

3-[3-(5-Hydroxypyrimidin-2-yl)benzyl]-1-(1-methyl-1H-pyrazol-4-yl)pyridazin-4(1H)-one (Example #187, 30 mg, 0.083 mmol), KOH (30 wt % in H₂O, 0.33 mL, 1.75 mmol) and MeCN (0.35 mL) were added to a microwave vial and cooled to −78° C. 2-Chloro-2,2,difluoroacetophenone (64 mg, 0.333 mmol) was added, the vial was sealed and the reaction heated to 80° C. for 1 hour. Brine was added and the products extracted into EtOAc. The organic phase was dried over Na₂SO₄, filtered and concentrated in vacuo. Purification of the residue by flash-chromatography (MPLC, 0-10% MeOH-EtOAc) gave 3-{3-[5-(difluoromethoxy)pyrimidin. 2-yl]benzyl}-1-(1-methyl-1H-pyrazol-4-yl)pyridazin-4(1H)-one as a-tan gum.

LRMS (ESI) calc'd for C20H17F2N6O2 [M+H]⁺: 411. Found: 411.

### Scheme 7

### Example #387

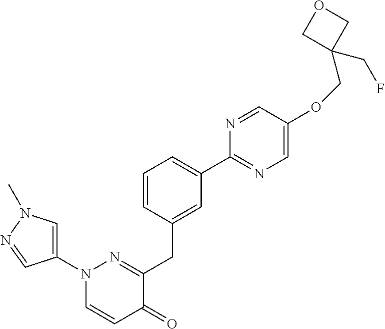

### 3-[3-(5-{[3-(Fluoromethyl)oxetan-3-yl]methoxy}pyrimidin-2-yl)benzyl]-1-(1-methyl-1H-pyrazol-4-yl)pyridazin-4(1H)-one

### Step 1. 3-[3-(5-{[3-(Fluoromethyl)oxetan-3-yl]methoxy}pyrimidin-2-yl)benzyl]-1-(1-methyl-1H-pyrazol-4-yl)pyridazin-4(1H)-one

[3-(5-{[3-(Hydroxymethyl)oxetan-3-yl]methoxy}pyrimidin-2-yl)benzyl]-1-(1-methyl-1H-pyrazol-4-yl)pyridazin-4(1H)-one (Example #268, 25 mg, 0.05 mmol) was dissolved in DCM (0.5 mL) and cooled to 0° C. DAST (0.015 mL, 0.08 mmol) was added dropwise and the reaction mixture was stirred for 1 hr. The reaction was quenched with NaHCO₃ at 0° C. until bubbling ceased. The mixture was then diluted with DCM and washed with saturated NaHCO₃. The organic layer was dried over Na₂SO₄, filtered and concentrated in vacuo. The crude residue was purified by reverse phase preparative HPLC (15-70% MeCN—H₂O, 0.05% TFA). Fractions containing the pure compound were collected and the free base was liberated using PL-HCO₃ cartridges (Stratospheres™, 0.9 mmol). The filtrate was frozen and freeze dried to afford 3-[3-(5-{[3-(fluoromethyl)oxetan-3-yl]methoxy}pyrimidin-2-yl)benzyl]-1-(1-methyl-1H-pyrazol-4-yl)pyridazin-4(1H)-one as a white solid.

LRMS (ESI) calc'd for C24H24FN6O3 [M+H]⁺: 463. Found: 463.

The following examples were prepared from Examples #365, 367 and a racemic mixture of Examples #378 and 379 according to Scheme 7 following similar procedures described for Example #387 which can be achieved by those of ordinary skill in the art of organic synthesis.

| | | IUPAC | Exact Mass |
|---|---|---|---|
| Example | Structure | Name | [M + H]+ |
| 388 | 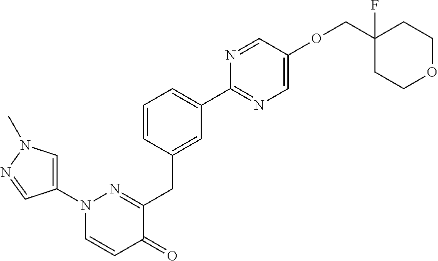 | 3-(3-{5-[(4- fluorotetrahydro- 2H-pyran-4- yl)methoxy] pyrimidin-2- yl}benzyl)-1- (1-methyl-1H- pyrazol-4- yl)pyridazin- 4(1H)-one | Calc'd 477, found 477 |
| 389 | 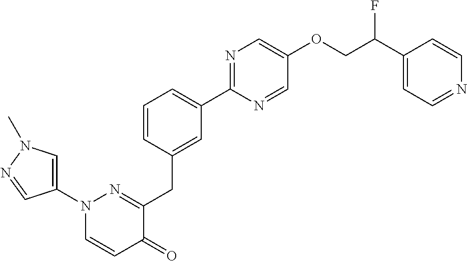 | rac-3-(3-{5-[2- fluoro-2- (pyridin-4- yl)ethoxy] pyrimidin-2- yl}benzyl)-1- (1-methyl-1H- pyrazol-4- yl)pyridazin- 4(1H)-one | Calc'd 484, found 484 |
| 390 | 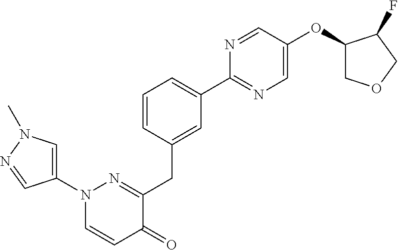 | rac-3-(3-{5- [(cis-4- fluorotetra- hydrofuran-3- yl}oxy] pyrimidin-2- yl}benzyl)-1- (1-methyl-1H- pyrazol-4- yl)pyridazin- 4(1H)-one | Calc'd 449, found 449 |
| | Cis, racemic | | |

### Scheme 8

### Intermediate #151

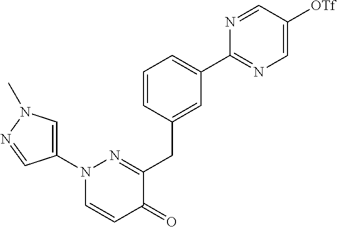

### 2-(3-{[1-(1-Methyl-1H-pyrazol-4-yl)-4-oxo-1,4-dihydropyridazin-3-yl]methyl}phenyl)pyrimidin-5-yl trifluoromethanesulfonate

### Step 1. 2-(3-{[1-(1-Methyl-1H-pyrazol-4-yl)-4-oxo-1,4-dihydropyridazin-3-yl]methyl}phenyl)pyrimidin-5-yl trifluoromethanesulfonate

To a solution of 3-[3-(5-hydroxypyrimidin-2-yl)benzyl]-1-(1-methyl-1H-pyrazol-4-yl)pyridazin-4(1H)-one (Example #187, 7.36 g, 20.4 mmol) in THF (102 mL) was added N-phenyl triflimide (8.03 g, 22.4 mmol) and DIPEA (4.28 mL, 24.5 mmol): The reaction mixture was stirred for 16 hr at room r.t., quenched with water and extracted with EtOAc (3×). The combined organic extracts were dried, the solvent was evaporated and the residue was purified by flash chromatography (MPLC, 0-20% MeOH-DCM) to provide 2-(3-{[1-(1-methyl-1H-5-pyrazol-4-yl)-4-oxo-1,4-dihydropyridazin-3-yl]methyl}phenyl)pyrimidin-5-yl trifluoromethanesulfonate.

LRMS (ESI) calc'd for C20H16F3N6O4S [M+H]⁺: 493. Found: 493.

The following intermediates were prepared from Example #188 and Intermediates #148-149 according to Scheme 8 following a similar procedure described for Intermediate #151, which can be achieved by those of ordinary skill in the art of organic synthesis.

| | | | Exact |
|---|---|---|---|
| | | | Mass |
| Intermediate | Structure | IUPAC Name | [M + H]+ |
| 152 | 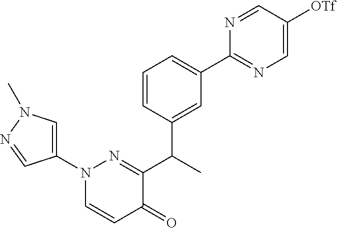 | 2-(3-{(1S or R)-1-[1-(1- methyl-1H-pyrazol-4-yl)-4- oxo-1,4-dihydropyridazin-3- yl]ethyl}phenyl)pyrimidin-5- yl trifluoromethanesulfonate | Calc'd 507, found 507 |
| | Enantiomer A | | |
| 153 | 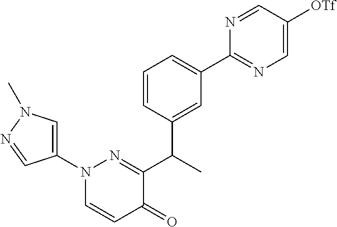 | 2-(3-{(1R or S)-1-[1-(1- methyl-1H-pyrazol-4-yl)-4- oxo-1,4-dihydropyridazin-3- yl]ethyl}phenyl)pyrimidin-5- yl trifluoromethanesulfonate | Calc'd 507, found 507 |
| | Enantiomer B | | |
| 154 | 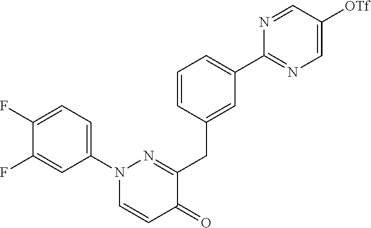 | 2-(3-{[1-(3,4- difluorophenyl)-4-oxo-1,4- dihydropyridazin-3- yl]methyl}phenyl)pyrimidin- 5-yl trifluoromethanesulfonate | Calc'd 525, found 525 |

### Scheme 8

### Example #391

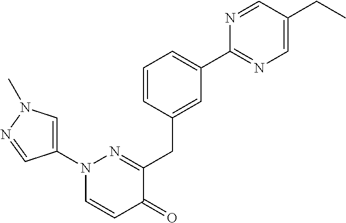

### 3-[3-(5-Ethylpyrimidin-2-yl)benzyl]-1-(1-methyl-1H-pyrazol-4-yl)pyridazin-4(1H)-one

### Step 1. 3-[3-(5-Ethylpyrimidin-2-yl)benzyl]-(1-methyl-1H-pyrazol-4-yl)pyridazin-4(1H)-one

A microwave vial was charged with 2-(3-{[1-(1-methyl-1H-pyrazol-4-yl)-4-oxo-1,4-dihydropyridazin-3-yl]methyl}phenyl)pyrimidin-5-yl trifluoromethanesulfonate (Intermediate #151, 440 mg, 0.89 mmol), potassium ethyltrifluoroborate (170 mg, 1.25 mmol), Cs₂CO₃ (873 mg, 2.68 mmol), PdCl₂(dppf).DCM (36.5 mg, 0.045 mmol), and toluene (2.6 mL) and water (894 μL) was added. The reaction-mixture was degassed by bubbling through N₂, and heated to 100° C. for 24 hours. The reaction mixture was filtered through Celite, washed with EtOAc, concentrated, and the residue was purified by flash chromatography (MPLC, 20-100% EtOAc-hexanes followed by 0-20% MeOH-DCM) to afford 3-[3-(5-ethylpyrimidin-2-yl)benzyl]-1-(1-methyl-1H-pyrazol-4-yl)pyridazin-4(1H)-one.

LRMS (ESI) calc'd for C21H21N6O [M+H]⁺: 373. Found: 373.

The following examples were prepared from Intermediates #151-154 according to Scheme 8 following similar procedures described for Example #391, which can be achieved by those of ordinary skill in the art of organic synthesis.

| | | IUPAC | Exact Mass |
|---|---|---|---|
| Example | Structure | Name | [M + H]+ |
| 392 | 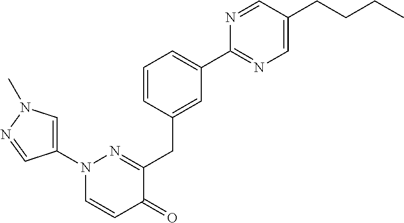 | 3-[3-(5- butylpyrimidin- 2- yl)benzyl]-1- (1-methyl-1H- pyrazol-4- yl)pyridazin- 4(1H)-one | Calc'd 401, found 401 |
| 393 | 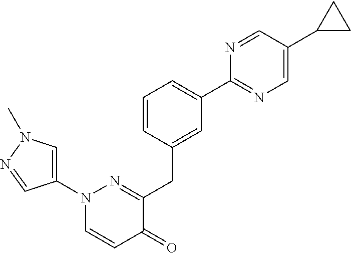 | 3-[3-(5- cyclopropyl- pyrimidin-2- yl)benzyl]-1- (1-methyl-1H- pyrazol-4- yl)pyridazin- 4(1H)-one | Calc'd 385, found 385 |
| 394 | 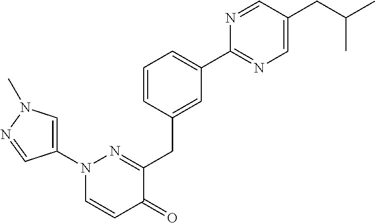 | 3-{3-[5-(2- methylpropyl) pyrimidin-2- yl)benzyl}-1- (1-methyl-1H- pyrazol-4- yl)pyridazin- 4(1H)-one | Calc'd 401, found 401 |
| 395 | 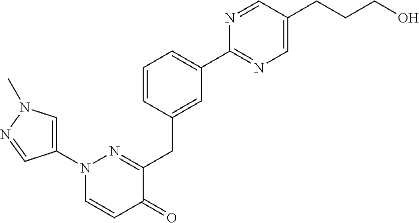 | 3-{3-[5-(3- hydroxypropyl) pyrimidin-2- yl]benzyl}-1- (1-methyl-1H- pyrazol-4- yl)pyridazin- 4(1H)-one | Calc'd 403, found 403 |
| 396 | 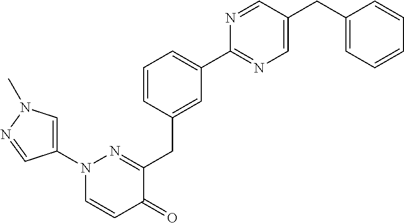 | 3-[3-(5- butylpyrimidin- 2- yl)benzyl]-1- (1-methyl-1H- pyrazol-4- yl)pyridazin- 4(1H)-one | Calc'd 435, found 435 |
| 397 | 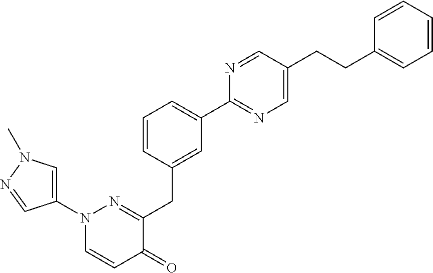 | 1-(1-methyl- 1H-pyrazol-4- yl)-3-{3-[5- (2- phenylethyl) pyrimidin-2- yl]benzyl} pyridazin- 4(1H)- one | Calc'd 449, found 449 |
| 398 | 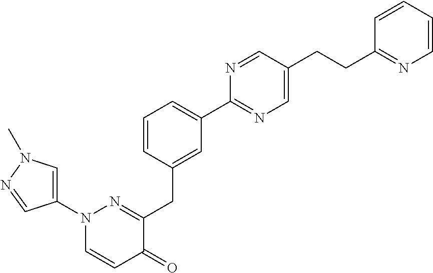 | 1-(1-methyl- 1H-pyrazol-4- yl)-3-(3-{5- [2-(pyridin-2- yl)ethyl] pyrimidin-2- yl}benzyl) pyridazin- 4(1H)- one | Calc'd 450, found 450 |
| 399 | 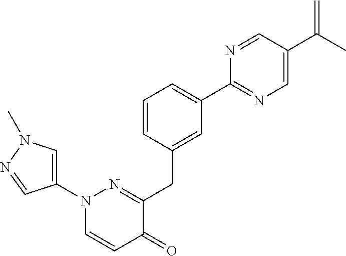 | 1-(1-methyl- 1H-pyrazol-4- yl)-3-{3-[5- (prop-1-en-2- yl)pyrimidin-2- yl]benzyl} pyridazin- 4(1H)- one | Calc'd 385, found 385 |
| 400 | 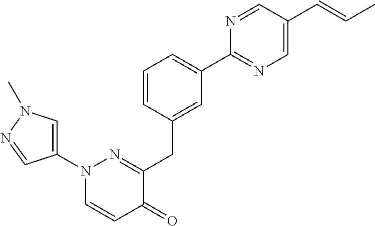 | 1-(1-methyl- 1H-pyrazol-4- yl)-3-(3-{5- [(1E)-prop-1- en-1- yl]pyrimidin-2- yl}benzyl) pyridazin- 4(1H)- one | Calc'd 385, found 385 |
| 401 | 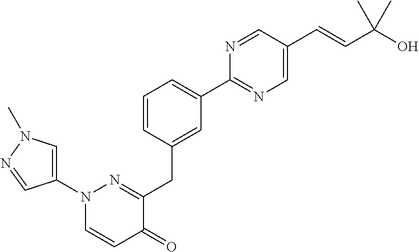 | 3-(3-{5-[(1E)- 3-hydroxy-3- methylbut-1- en-1- yl]pyrimidin- 2-yl}benzyl)- 1-(1-methyl- 1H-pyrazol-4- yl)pyridazin- 4(1H)-one | Calc'd 429, found 429 |
| 402 | 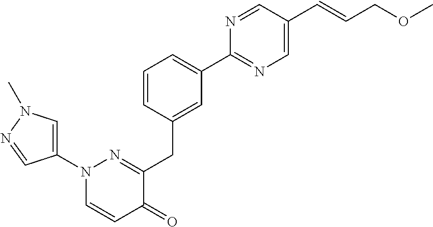 | 3-(3-{5-[(1E)- 3- methoxyprop- 1-en-1- yl]pyrimidin- 2-yl}benzyl)- 1-(1-methyl- 1H-pyrazol-4- yl)pyridazin- 4(1H)-one | Calc'd 415, found 415 |
| 403 | 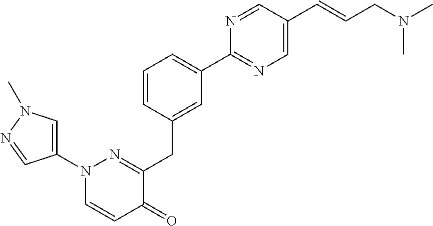 | 3-(3-{5-[(1E)- 3- (dimethylamino) prop-1-en-1- yl]pyrimidin- 2-yl}benzyl)- 1-(1-methyl- 1H-pyrazol-4- yl)pyridazin- 4(1H)-one | Calc'd 428, found 428 |
| 404 | 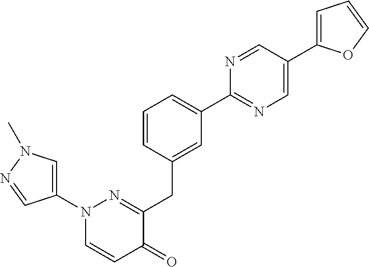 | 3-{3-[5- (furan-2- yl)pyrimidin- 2-yl]benzyl}- 1-(1-methyl- 1H-pyrazol-4- yl)pyridazin- 4(1H)-one | Calc'd 411, found 411 |
| 405 | 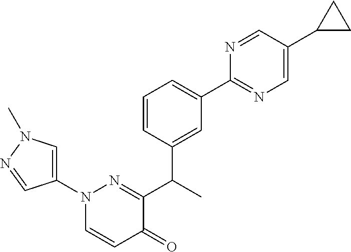 | 3-{(1S or R)- 1-[3-(5- cyclopropyl- pyrimidin-2- yl)phenyl] ethyl}-1-(1- methyl-1H- pyrazol-4- yl)pyridazin- 4(1H)-one | Calc'd 399, found 399 |
| | Enantiomer A | | |
| 406 | 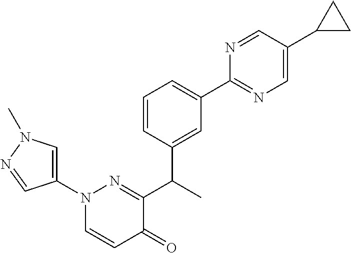 | 3-{(1R or S)- 1-[3-(5- cyclopropyl- pyrimidin-2- yl)phenyl] ethyl}-1-(1- methyl-1H- pyrazol-4- yl)pyridazin- 4(1H)-one | Calc'd 399, found 399 |
| | Enantiomer B | | |
| 407 | 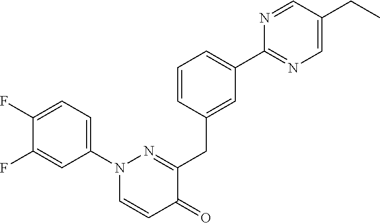 | 1-(3,4- difluorophenyl)- 3-[3-(5- ethylpyrimidin- 2- yl)benzyl] pyridazin-4(1H)- one | Calc'd 405, found 405 |

### Scheme 8

### Example #408

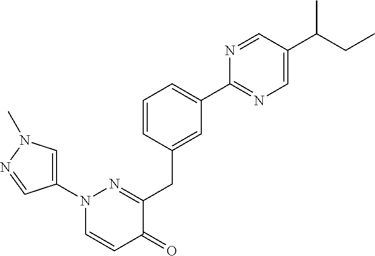

### rac-3-{3-[5-(Butan-2-yl)pyrimidin-2-yl]benzyl}-1-(1-methyl-1H-pyrazol-4-yl)pyridazin-4(1-1)-one

### Step 1. rac-3-{3-[5-(Butan-2-yl)pyrimidin-2-yl]benzyl}-1-(1-methyl-1H-pyrazol-4-yl)pyridazin-4(1H)-one

Zinc (93 mg, 1.42 mmol) was taken up in DMA (0.2 mL) and to the slurry a crystal of iodine (I₂) was added. The mixture was shaken on a vortexer for two minutes and 2-iodobutane (131 mg, 0.71 mmol) was added. The reaction mixture was heated to 80° C. for three hours and filtered to give a solution of the zinc iodide. 2-(3-{[1-(1-Methyl-1H-pyrazol-4-yl)-4-oxo-1,4-dihydropyridazin-3-yl]methyl}phenyl)pyrimidin-5-yl trifluoroomethanesulfonate (Intermediate #151, 70 mg, 0.142 mmol) was dissolved in THF (400 μL) and 2-dicyclohexyphosphino-2′,6′-dimethoxybiphenyl (5.9 mg, 0.014 mmol) was added. The reaction solution was degassed by passing through a stream of Ar for five minutes. Subsequently, Pd(OAc)₂ (1.6 mg, 7.1 μmol) was added followed by the filtered zinc iodide solution. The reaction mixture was diluted with EtOAc (few drops of MeOH added) and water. The biphasic reaction solution was filtered through diatomaceous earth, the phases were separated, the aqueous layer was extracted with EtOAc, the combined organics were dried over Na₂SO₄, filtered, the solvent was evaporated and the residue was purified by flash chromatography (MPLC, MeOH-DCM) to give rac-3-{3-[5-(butan-2-yl)pyrimidin-2-yl]benzyl}-1-(1-methyl-1H-pyrazol-4-yl)pyridazin-4(1H)-one.

LRMS (ESI) calc'd for C23H25N6O [M+H]⁺: 401. Found: 401.

### Scheme 8

### Example #409

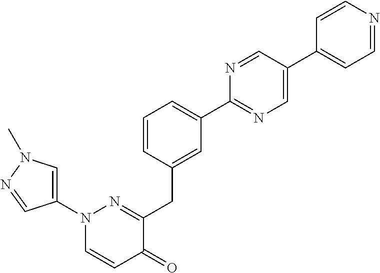

### 1-(1-Methyl-1H-pyrazol-4-yl)-3-{3-[5-(pyridin-4-yl)pyrimidin-2-yl]benzyl}pyridazin-4(1H)-one

### Step 1. 1-(1-Methyl-1H-pyrazol-4-yl)-{3-[5-(pyridin-4-yl)pyrimidin-2-yl]benzyl}pyridazin-4(1H)-one

A microwave vial was charged with 2-(3-{[1-(1-methyl-1H-pyrazol-4-yl)-4-oxo-1,4-dihydropyridazin-3-yl]methyl}phenyl)pyrimidin-5-yl trifluoromethanesulfonate (Intermediate #151, 20 mg, 0.04 mmol), 4-(4,4,5,5-tetramethyl-1,3,2-dioxaborolan-2-yl)pyridine (9.9 mg, 0.05 mmol), KF (7.0 mg. 0.12 mmol) and RuPhos (3.8 mg, 8.1 μmol). Degassed 1,4-dioxane (1.4 mL) and water (203 μL) were added. The reaction mixture was further degassed by bubbling N₂ through and was kept at 80° C. for 24 hours. The reaction mixture was filtered through Celite, the solvent was evaporated in vacuo, the residue was dissolved in MeCN/water/DMSO and purified by mass directed reverse phase HPLC. The solution was filtered through a MP-HCO₃ column (Stratospheres™, 0.9 mmol) to remove the TFA and the solvent was removed by centrifugation to provide 1-(1-methyl-1H-pyrazol-4-yl)-3-{3-[5-(pyridin-4-yl)pyrimidin-2-yl]benzyl}pyridazin-4(1H)-one.

LRMS (ESI) calc'd for C24H20N7O [M+H]⁺: 422. Found: 422.

The following examples were prepared according to Scheme 8 following a similar procedure described for Example #409, which can be achieved by those of ordinary skill in the art of organic synthesis.

| | | IUPAC | Exact Mass |
|---|---|---|---|
| Example | Structure | Name | [M + H]+ |
| 410 | 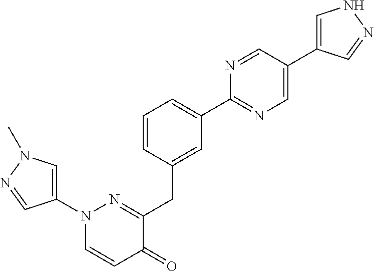 | 1-(1-methyl- 1H-pyrazol-4- yl)-3-{3-[5- (1H-pyrazol- 4- yl)pyrimidin- 2- yl]benzyl} pyridazin- 4(1H)- one | Calc'd 411, found 411 |
| 411 | 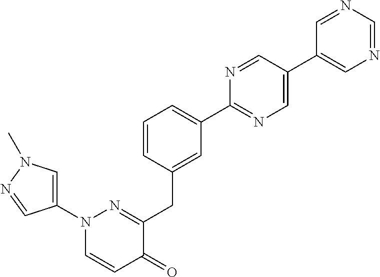 | 3-[3-(5,5′- bipyrimidin- 2-yl)benzyl]- 1-(1-methyl- 1H-pyrazol-4- yl)pyridazin- 4(1H)-one | Calc'd 423, found 423 |
| 412 | 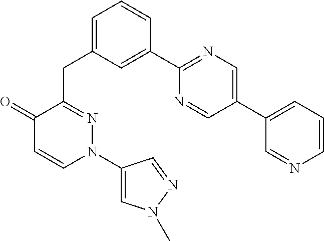 | 1-(1-methyl- 1H-pyrazol-4- yl)-3-[3-(5- pyridin-3- ylpyrimidin- 2- yl)benzyl] pyridazin- 4(1H)- one | Calc'd 422, found 422 |
| 413 | 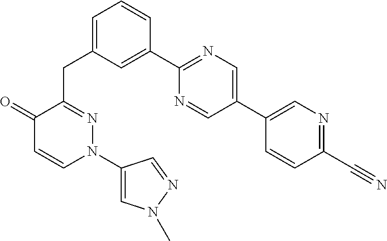 | 5-[2-(3-{[1- (1-methyl-1H- pyrazol-4-yl)- 4-oxo-1,4- dihydropyrida- zin-3- yl]methyl} phenyl) pyrimidin- 5-yl]pyridine- 2-carbonitrile | Calc'd 447, found 447 |
| 414 | 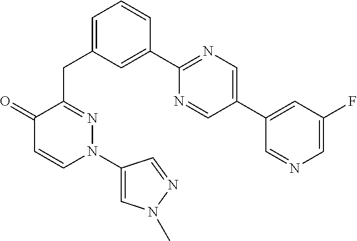 | 3-{3-[5-(5- fluoropyridin- 3- yl)pyrimidin- 2-yl]benzyl}- 1-(1-methyl- 1H-pyrazol-4- yl)pyridazin- 4(1H)-one | Calc'd 440, found 440 |
| 415 | 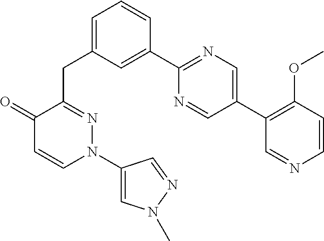 | 3-{3-[5-(3- methoxypyridin- 4- yl)pyrimidin- 2-yl]benzyl}- 1-(1-methyl- 1H-pyrazol-4- yl)pyridazin- 4(1H)-one | Calc'd 452, found 452 |
| 416 | 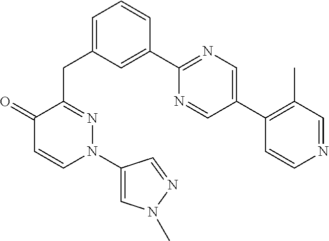 | 1-(1-methyl- 1H-pyrazol-4- yl)-3-{3-[5- (3- methylpyridin- 4- yl)pyrimidin- 2- yl]benzyl} pyridazin-4(1H)- one | Calc'd 436, found 436 |
| 417 | 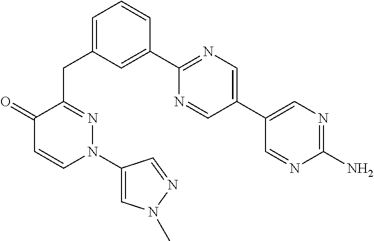 | 3-[3-(2′- amino-5,5′- bipyrimidin- 2-yl)benzyl]- 1-(1-methyl- 1H-pyrazol-4- yl)pyridazin- 4(1H)-one | Calc'd 438, found 438 |
| 418 | 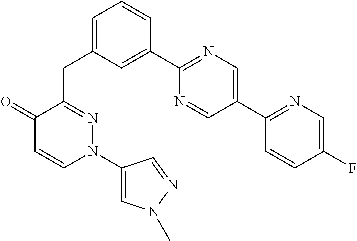 | 3-{3-[5-(5- fluoropyridin- 2- yl)pyrimidin- 2-yl]benzyl}- 1-(1-methyl- 1H-pyrazol-4- yl)pyridazin- 4(1H)-one | Calc'd 440, found 440 |
| 419 | 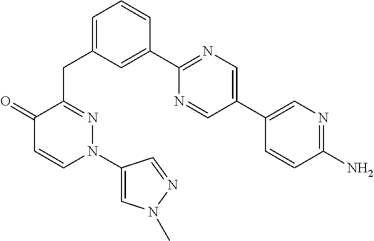 | 3-{3-[5-(6- aminopyridin- 3- yl)pyrimidin- 2-yl]benzyl}- 1-(1-methyl- 1H-pyrazol-4- yl)pyridazin- 4(1H)-one | Calc'd 437, found 437 |
| 420 | 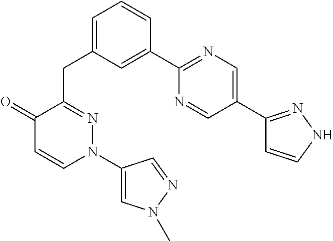 | 1-(1-methyl- 1H-pyrazol-4- yl)-3-{3-[5- (1H-pyrazol- 3- yl)pyrimidin- 2- yl]benzyl} pyridazin-4(1H)- one | Calc'd 411, found 411 |
| 421 | 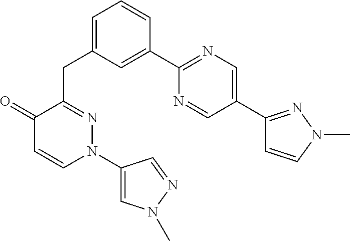 | 1-(1-methyl- 1H-pyrazol-4- yl)-3-{3-[5- (1-melhyl-1H- pyrazol-3- yl)pyrimidin- 2- yl]benzyl} pyridazin-4(1H)- one | Calc'd 425, found 425 |
| 422 | 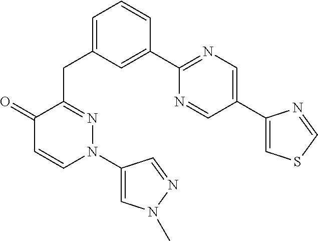 | 1-(1-methyl- 1H-pyrazol-4- yl)-3-{3-[5- (1,3-thiazol-4- yl)pyrimidin- 2- yl]benzyl} pyridazin-4(1H)- one | Calc'd 428, found 428 |
| 423 | 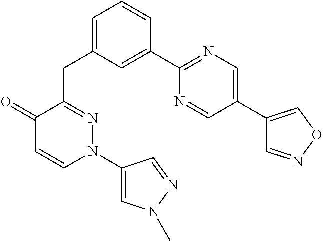 | 3-[3-(5- isoxazol-4- ylpyrimidin-2- yl)benzyl]-1- (1-methyl-1H- pyrazol-4- yl)pyridazin- 4(1H)-one | Calc'd 412, found 412 |
| 424 | 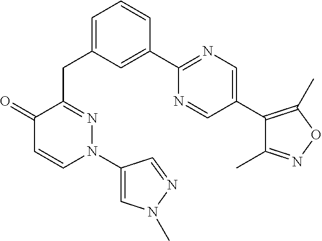 | 3-{3-[5-(3,5- dimethylisoxa- zol-4- yl)pyrimidin- 2-yl]benzyl}- 1-(1-methyl- 1H-pyrazol-4- yl)pyridazin- 4(1H)-one | Calc'd 440, found 440 |
| 425 | 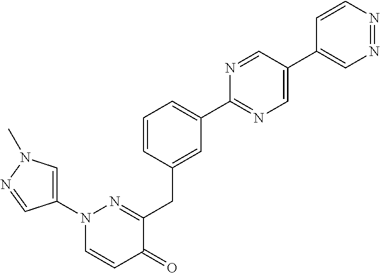 | 1-(1-methyl- 1H-pyrazol-4- yl)-3-[3-(5- pyridazin-4- ylpyrimidin-2- yl)benzyl] pyridazin-4(1H)- one | Calc'd 423, found 423 |

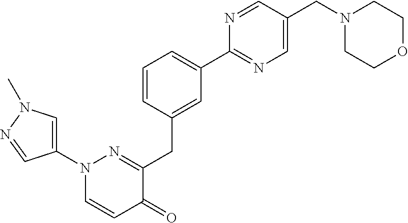

### 1-(1-Methyl-1H-pyrazol-4-yl)-3-{3-[5-(morpholin-4-ylmethyl)pyrimidin-2-yl]benzyl}pyridazin-4(1H)-one

### Step 1. 1-(1-Methyl-1H-pyrazol-4-yl)-3-{3-[5-(morpholin-4-ylmethyl)pyrimidin-2-yl]benzyl}pyridazin-4(1H-one

A microwave vial was charged with 2-(3-{[1-(1-methyl-1H-pyrazol-4-yl)-4-oxo-1,4-dihydropyridazin-3-yl]methyl}phenyl)pyrimidin-5-yl trifluoromethanesulfonate (Intermediate #151, 20 mg, 0.041 mmol), potassium trifluoro(morpholin-4-ylmethyl)borate (10.0 mg, 0.049 mmol), XPhos (3.9 mg; 8.1 μmol), Pd₂(dba)₃ (3.7 mg, 4.0 μmol), Cs₂CO₃ (39 mg, 0.12 mmol) and degassed THF (355 μL) and water (50 μL) were added. The reaction mixture was further degassed by bubbling N₂ through and was kept at 80° C. for 24 hours. The reaction mixture was filtered through Celite, the solvent was evaporated and the residue was dissolved in MeCN/Water/DMSO and purified by mass directed reverse phase HPLC. The solution was filtered through MP-HCO₃ (Stratospheres™, 0.9 mmol) column to remove the TFA, and the solvent was removed by centrifugation to provide 1-(1-methyl-1H-pyrazol-4-yl)-3-{3-[5-(morpholin-4-ylmethyl)pyrimidin-2-yl]benzyl}pyridazin-4(1H)-one.

LRMS (ESI) calc'd for C24H26N7O2 [M+H]⁺: 444. Found: 444.

The following examples were prepared according to Scheme 8 following a similar procedure described for Example #426, which can be achieved by those of ordinary skill in the art of organic synthesis.

| | | IUPAC | Exact Mass |
|---|---|---|---|
| Example | Structure | Name | [M + H]+ |
| 427 | 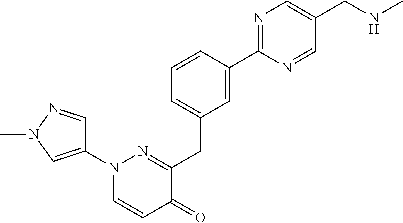 | 3-(3-{5- [(methylamino) methyl]pyrimidin- 2- yl}benzyl)-1- (1-methyl-1H- pyrazol-4- yl)pyridazin- 4(1H)-one | Calc'd 388 found 388 |
| 428 | 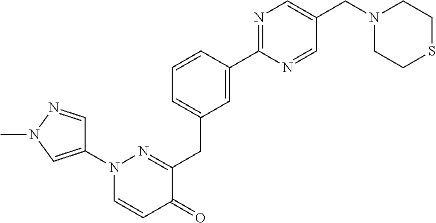 | 1-(1-methyl- 1H-pyrazol-4- yl)-3-{3-[5- (thiomorpholin- 4- ylmethyl) pyrimidin-2- yl]benzyl} pyridazin-4(1H)- one | Calc'd 460, found 460 |
| 429 | 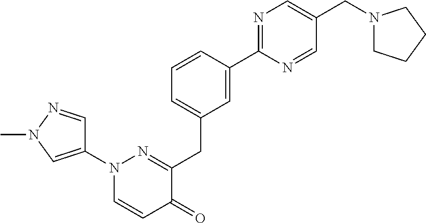 | 1-(1-methyl- 1H-pyrazol-4- yl)-3-{3-[5- (pyrrolidin-1- ylmethyl) pyrimidin-2- yl]benzyl} pyridazin-4(1H)- one | Calc'd 428, found 428 |
| 430 | 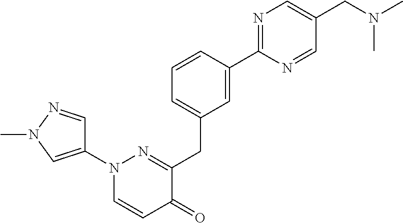 | 3-(3-{5- [(dimethylamino) methyl]pyrimidin- 2- yl}benzyl)-1- (1-methyl-1H- pyrazol-4- yl)pyridazin- 4(1H)-one | Calc'd 402, found 402 |
| 431 | 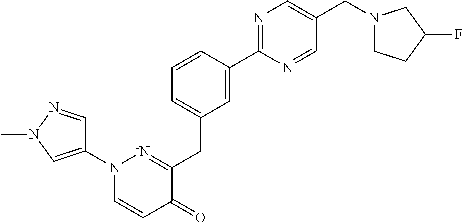 | rac-3-(3-{5- [(3- fluoropyrrolidin- 1- yl)methyl] pyrimidin-2- yl}benzyl)-1- (1-methyl-1H- pyrazol-4- yl)pyridazin- 4(1H)-one | Calc'd 446, found 446 |
| 432 | 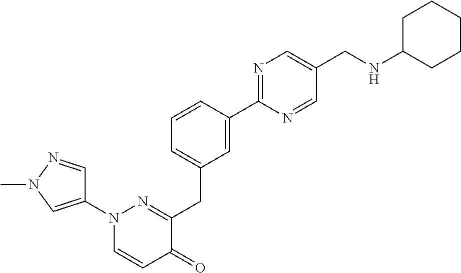 | 3-(3-{5- [(cyclohexyl- amino)methyl] pyrimidin- 2- yl}benzyl)-1- (1-methyl-1H- pyrazol-4- yl)pyridazin- 4(1H)-one | Calc'd 456, found 456 |

The following intermediates were prepared according to Scheme 8 following a similar procedure described for Example #426, which can be achieved by those of ordinary skill in the art of organic synthesis.

| | | | Exact |
|---|---|---|---|
| | | | Mass |
| Intermediate | Structure | IUPAC Name | [M + H]+ |
| 155 | 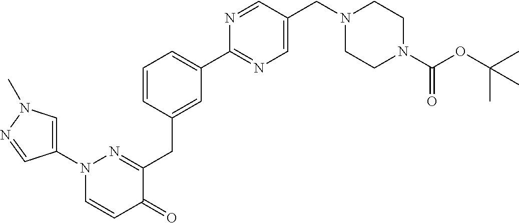 | tert-butyl 4-{[2- (3-{[1-(1- methyl-1H- pyrazol-4-yl)-4- oxo-1,4- dihydropyridazin- 3- yl]methyl}phenyl) pyrimidin-5- yl]methyl} piperazine-1- carboxylate | nd |
| 156 | 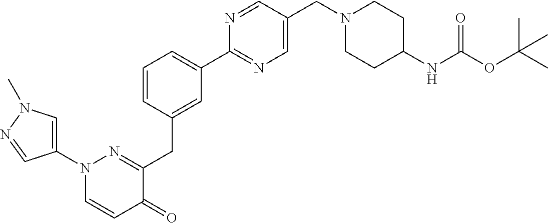 | tert-butyl (1- {[2-(3-{[1-(1- methyl-1H- pyrazol-4-yl)-4- oxo-1,4- dihydropyridazin- 3- yl]methyl}phenyl) pyrimidin-5- yl]methyl} piperidin-4- yl)carbamate | nd |

### Scheme 8

### Example #433

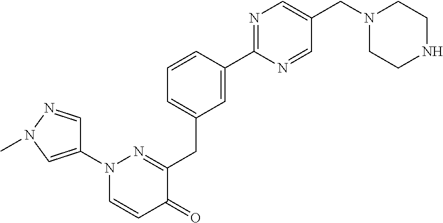

### 1-(1-Methyl-1H-pyrazol-4-yl)-3-{3-[5-(1-oxetan-3-yl-1H-pyrazol-4-yl)pyrimidin-2-yl]benzyl}pyridazin-4(1H)-one

### Step 1. 1-(1-Methyl-1H-pyrazol-4-yl)-3-{3-[5-(1-oxetan-3-yl-1H-pyrazol-4-yl)pyrimidin-2-yl]benzyl}pyridazin-4(1H)-one

tert-Butyl 4-{[2-(3-{([1-(1-methyl-1H-pyrazol-4-yl)-4-oxo-1,4-dihydropyridazin-3-yl]methyl}phenyl)pyrimidin-5-yl]methyl}piperazine-1-carboxylate (Intermediate #155, crude product, theoretical yield 0.081 mmol) was taken up in DCM (0.5 mL) and TFA (0.5 mL) was added. The mixture was stirred at r.t. for 1 hour. The solvent was removed in vacuo and the residue purified by mass triggered reverse phase preparative HPLC to give 1-(1-methyl-1H-pyrazol-4-yl)-3-{3-[5-(1-oxetan-3-yl-1H-pyrazol-4-yl)pyrimidin-2-yl]benzyl}pyridazin-4(1H)-one.

LRMS (ESI) calc'd for C24H27N8O [M+H]⁺: 443. Found 443.

The following example was prepared from Intermediate #156 according to Scheme 8 following a similar procedure described for Example #433, which can be achieved by those of ordinary kill in the art of organic synthesis.

| | | IUPAC | Exact Mass |
|---|---|---|---|
| Example | Structure | Name | [M + H]+ |
| 434 | 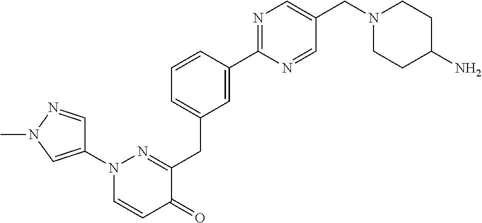 | 3-(3-{5-[(4- aminopiperidin- 1- yl)methyl] pyrimidin-2- yl}benzyl)-1- (1-methyl-1H- pyrazol-4- yl)pyridazin- 4(1H)-one | Calc'd 457, found 457 |

### Scheme 8

### Example #435

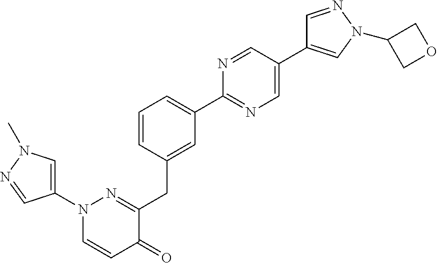

### 1-(1-Methyl-1H-pyrazol-4-yl)-3-{3-[5-(1-oxetan-3-yl-1H-pyrazol-4-yl)pyrimidin-2-yl]benzyl}pyridazin-4(1H)-one

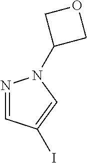

### Step 1. 4-Iodo-1-(oxetan-3-yl)-1H-pyrazole

3-Iodooxetane (1.0 g, 5.44 mmol), 4-iodopyrazole (1.160 g, 5.98 mmol); and Cs₂CO₃ (1.95 g, 5.98 mmol) were combined in a 20 mL microwave vial. The vial was evacuated and back-filled with N₂ gas (3×) before adding DMF-(16 mL). The mixture was heated to 150° C. for 20 minutes under microwave irradiation (Biotage, Initiator). Saturated NH₄Cl was added and the products extracted into EtOAc (3×). The combined organic layers were then washed with brine, dried over MgSO₄, filtered and concentrated in vacuo. Purification of the residue by flash chromatography (MPLC, 0-40% EtOAc-hexanes) gave 4-iodo-1-(oxetan-3-yl)-1H-pyrazole.

LRMS (ESI) calc'd for C6H81N2O [M+H]⁺: 251. Found 251.

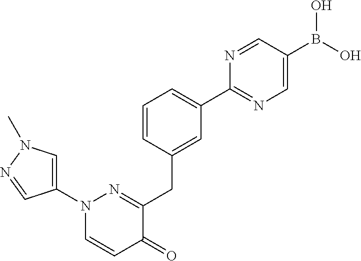

### Step 2. [2-(3-{[1-(1-Methyl-1-pyrazol-4-yl)-4-oxo-1, 4-dihydropyridazin-3-yl]methyl}phenyl)pyrimidin-5-yl]boronic acid

2-(3-{[1-(1-Methyl-1H-pyrazol-4-yl)-4-oxo-1,4-dihydropyridazin-3-yl]methyl}phenyl)pyrimidin-5-yl trifluoromethanesulfonate (Intermediate #151, 100 mg, 0.203 mmol), B₂Pin₂ (77 mg, 0.305 mmol), Pd₂(dba)₃ (9.3 mg, 10.2 μmol), X-Phos 7.7 mg, 0.016 mmol) and potassium acetate (60 mg, 0.609 mmol) were combined in a 5 mL microwave vial. The vial was evacuated and back-filled with N₂ gas (3×) before adding 1,4-dioxane (3 mL). The reaction mixture was allowed to stir at 100° C. for 2 hours. The reaction mixture was filtered through Celite and concentrated in vacuo to give [2-(3-{[1-(1-methyl-1H-pyrazol-4-yl)-4-oxo-1,4-dihydropyridazin-3-yl]methyl}phenyl)pyrimidin-5-yl]boronic acid.

LRMS (ESI) calc'd for C19H18BN6O3 [M+H]⁺: 389. Found 389.

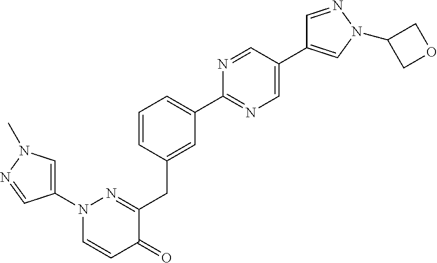

### Step 3. 1-(1-Methyl-1H-pyrazol-4-yl)-3-{3-[5-(1-oxetan-3-yl-1H-pyrazol-4-yl)pyrimidin-2-yl]benzyl}pyridazin-4(1H)-one

To a 2 mL microwave vial was added [2-(3-{[1-(1-methyl-1H-pyrazol-4-yl)-4-oxo-1,4-dihydropyridazin-3-yl]methyl}phenyl)pyrimidin-5-yl]boronic acid (50 mg, 0.129 mmol), 4-iodo-1-(oxetan-3-yl)-1H-pyrazole (48 mg, 0.193 mmol), Pd₂(dba)₃ (5.9 mg, 6.44 μmol), X-Phos (4.9 mg, 10.30 μmol), CS₂CO₃ (126 mg, 0.386 mmol) and 1,4-dioxane (1.5 mL). N₂ gas was bubbled through the reaction mixture for 10 minutes before heating to 100° C. for 1 hour. Room temperature was attained and the mixture filtered through Celite eluting with EtOAc and DCM. The filtrate was concentrated in vacuo and the residue purified by flash chromatography (MPLC, 0-15% MeOH-EtOAc) to give 1-(1-methyl-1H-pyrazol-4-yl)-3-{3-[5-(1-oxetan-3-yl-1H-pyrazol-4-yl)pyrimidin-2-yl]benzyl}pyridazin-4(1H)-one.

LRMS (ESI) calc'd for C25H23N8O2 [M+H]⁺: 467. Found 467.

### Scheme 8

### Example #436

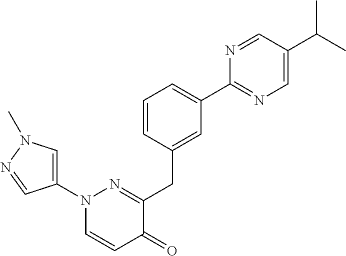

### 1-(1-Methyl-1H-pyrazol-4-yl)-3-{3-[5-(propan-2-yl)pyrimidin-2-yl]benzyl}pyridazin-4(1H)-one

### Step 1. 1-(1-Methyl-1H-pyrazol-4-yl)-3-{3-[5-propan-2-yl)pyrimidin-2-yl]benzyl}pyridazin-4(1H)-one

To a solution of 1-(1-methyl-1H-pyrazol-4-yl)-3-{3-[5-(prop-1-en-2-yl)pyrimidin-2-yl]benzyl}pyridazin-4(1H)-one (Example #399, 20 mg; 0.052 mmol) in EtOAc (260 μL) and MeOH (260 μL) was added Pd/C (10 wt %, 1.5 mg, 0.014 mmol) under N₂. Subsequently, H₂ was bubbled through the reaction mixture for 3 minutes. The reaction was stirred under H₂ atmosphere for 18 hours, was filtered through Celite and purified by flash chromatography (MPLC, 0-20% MeOH-DCM) to provide 1-(1-methyl-1H-pyrazol-4-yl)-3-{3-[5-(propan-2-yl)pyrimidin-2-yl]benzyl}pyridazin-4(1H)-one.

LRMS (ESI) calc'd for C22H23N6O [M+H]⁺: 387. Found: 387.

The following examples were prepared from Examples #401-403 according to Scheme 8 following similar procedures described for Example #436, which can be achieved by those of ordinary skill in the art of organic synthesis.

| | | IUPAC | Exact Mass |
|---|---|---|---|
| Example | Structure | Name | [M + H]+ |
| 437 | 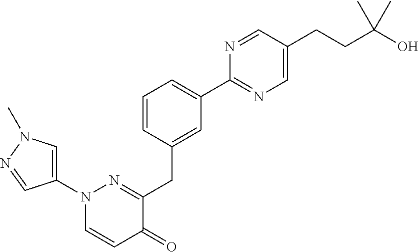 | 3-{3-[5-(3- hydroxy-3- methylbutyl) pyrimidin-2- yl]benzyl}-1- (1-methyl-1H- pyrazol-4- yl)pyridazin- 4(1H)-one | Calc'd 431, found 431 |
| 438 | 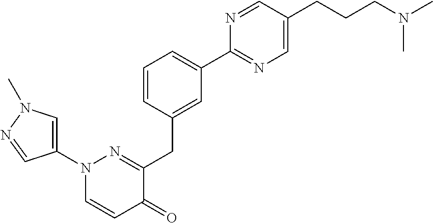 | 3-(3-{5-[3- (dimethylamino) propyl] pyrimidin-2- yl}benzyl)-1- (1-methyl-1H- pyrazol-4- yl)pyridazin- 4(1H)-one | Calc'd 430, found 430 |
| 439 | 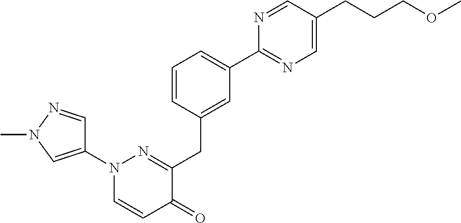 | 3-{3-[5-(3- methoxypropyl) pyrimidin-2- yl]benzyl}-1- (1-methyl-1H- pyrazol-4- yl)pyridazin- 4(1H)-one | Calc'd 417, found 417 |

### Scheme 8

### Example #440

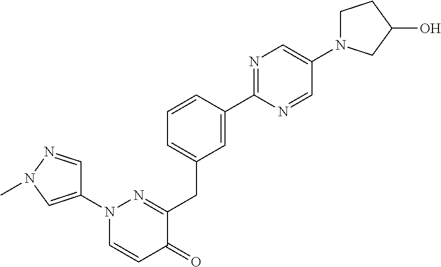

### rac-3-{3-[5-(3-Hydroxypyrrolidin-1-yl)pyrimidin-2-yl]benzyl}-1-(1-methy 1H-pyrazol-4-yl)pyridazin-4(1H)-one

### Step 1. rac-3-{3-[5-(3-Hydroxypyrrolidin-1-yl)pyrimidin-2-yl]benzyl}-1-methy 1H-pyrazol-4-yl pyridazin-4(1H)-one

To a 2 mL microwave vial was added 2-(3-{[1-(1-methyl-1H-pyrazol-4-yl)-4-oxo-1,4-dihydropyridazin-3-yl]methyl}phenyl)pyrimidin-5-yl trifluoromethanesulfonate (Intermediate #151, 40 mg, 0.081 mmol) dissolved in NMP (0.5 mL), followed by pyrrolidin-3-ol (34 mg, 0.40 mmol). The vial was sealed and heated to 200° C. for 5 minutes under microwave irradiation (Biotage, Initiator). The reaction mixture was filtered, then purified by mass-triggered reverse phase preparative HPLC to provide rac-3-{3-[5-(3-hydroxypyrrolidin-1-yl)pyrimidin-2-yl]benzyl}-1-(1-methyl-1H-pyrazol-4-yl)pyridazin-4(1H)-one.

LRMS calc'd for C23H24N7O2 [M+H]⁺: 430. Found: 430.

The following examples were prepared according to Scheme 8 following similar procedures described for Example #440, which can be achieved by those of ordinary skill in the art of organic synthesis.

| | | IUPAC | Exact Mass |
|---|---|---|---|
| Example | Structure | Name | [M + H]+ |
| 441 | 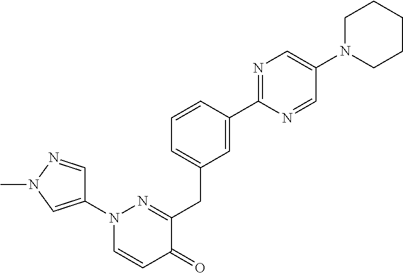 | 1-(1-methyl- 1H-pyrazol-4- yl)-3-[3-(5- piperidin-1- ylpyrimidin-2- yl)benzyl] pyridazin- 4(1H)-one | Calc'd 428, found 428 |
| 442 | 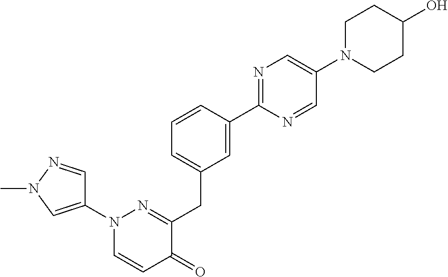 | 3-{3-[5-(4- hydroxypiperidin- 1- yl)pyrimidin- 2-yl]benzyl}- 1-(1-methyl- 1H-pyrazol-4- yl)pyridazin- 4(1H)-one | Calc'd 444, found 444 |
| 443 | 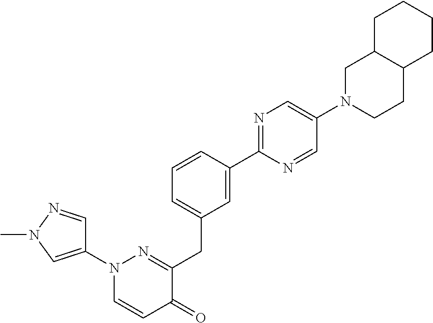 | 1-(1-methyl- 1H-pyrazol-4- yl)-3-{3-[5- (octahydroiso- quinolin- 2(1H)- yl)pyrimidin- 2- yl]benzyl} pyridazin-4(1H)- one | Calc'd 482, found 482 |
| | Mixture of isomers | | |
| 444 | 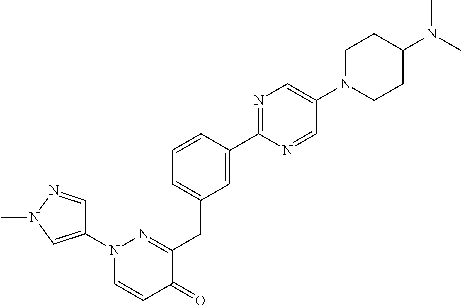 | 3-(3-{5-[4- (dimethylamino) piperidin- 1- yl]pyrimidin- 2-yl}benzyl)- 1-(1-methyl- 1H-pyrazol-4- yl)pyridazin- 4(1H)-one | Calc'd 471, found 471 |
| 445 | 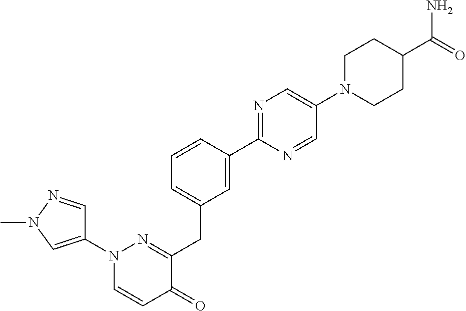 | 1-[2-(3-{[1- (1-methyl-1H- pyrazol-4-yl)- 4-oxo-1,4- dihydropyridazin- 3- yl]methyl}phenyl) pyrimidin- 5- yl]piperidine- 4- carboxamide | Calc'd 471, found 471 |
| 446 | 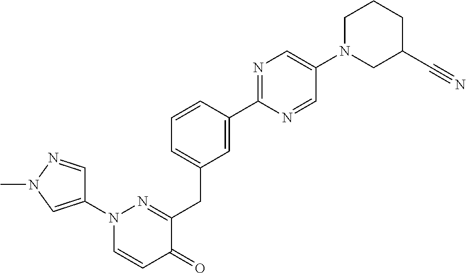 | rac-1-[2-(3- {[1-(1- methyl-1H- pyrazol-4-yl)- 4-oxo-1,4- dihydropyridazin- 3- yl]methyl}phenyl) pyrimidin- 5- yl]piperidine- 3-carbonitrile | Calc'd 453, found 453 |
| 447 | 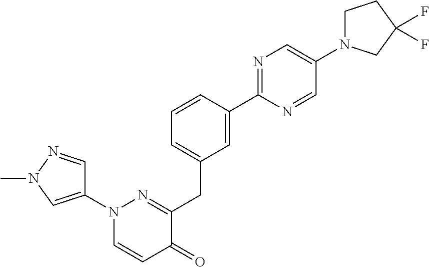 | 3-{3-[5-(3,3- difluoropyrrolidin- 1- yl)pyrimidin- 2-yl]benzyl}- 1-(1-methyl- 1H-pyrazol-4- yl)pyridazin- 4(1H)-one | Calc'd 450, found 450 |
| 448 | 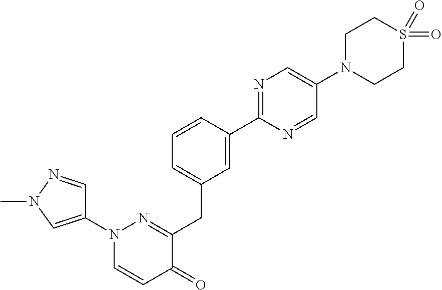 | 3-{3-[5-(1,1- dioxidothio- morpholin-4- yl]pyrimidin- 2-yl]benzyl}- 1-(1-methyl- 1H-pyrazol-4- yl)pyridazin- 4(1H)-one | Calc'd 478, found 478 |
| 449 | 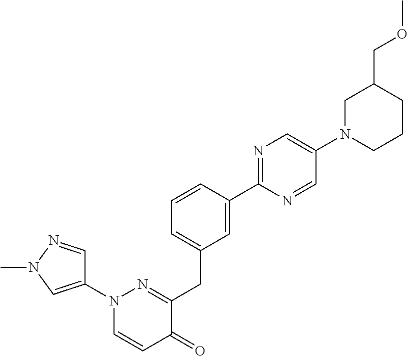 | rac-3-(3-{5- [3- (methoxymethyl) piperidin-1- yl]pyrimidin- 2-yl}benzyl)- 1-(1-methyl- 1H-pyrazol-4- yl)pyridazin- 4(1H)-one | Calc'd 472, found 472 |
| 450 | 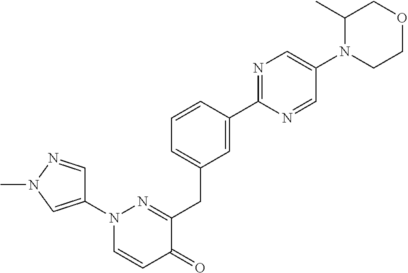 | rac-3-{3-[5- (3- methylmorpholin- 4- yl)pyrimidin- 2-yl]benzyl}- 1-(1-methyl- 1H-pyrazol-4- yl)pyridazin- 4(1H)-one | Calc'd 444, found 444 |

### Scheme 9

### Example #451

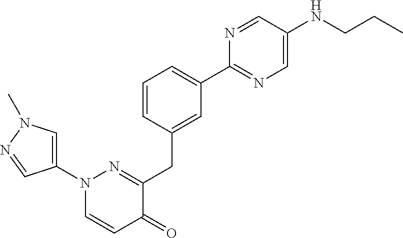

### 1-(1-Methyl-1H-pyrazol-4-yl)-3-{3-[5-(propylamino)pyrimidin-2-yl]benzyl}pyridazin-4(1H)-one

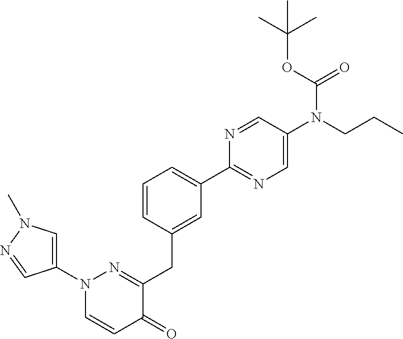

### Step 1. tert-Butyl [2-(3-{[1-(1-methyl-1H-pyrazol-4-yl)-4-oxo-1,4-dihydropyridzin-3-yl]methyl}phenyl)pyrimidin-5-yl]propylcarbamate

A microwave vial was charged with tert-butyl [2-(3-{[1-(1-methyl-1H-pyrazol-4-yl)-4-oxo-1,4-dihydropyridazin-3-yl]methyl}phenyl)pyrimidin-5-yl]carbamate (Example #159, 120 mg, 0.26 mmol), iodopropane (89 μL, 0.91 mmol), Cs₂CO₃ (255 mg, 0.78 mmol) and DMF (2.6 mL). The reaction mixture was heated to 80° C. for 3 hrs, diluted with water and extracted with EtOAc (3×). The combined organic phases were dried over Na₂SO₄, filtered and the solvent was removed in vacuo to provide tert-butyl [2-(3-{[1-(1-methyl-1H-pyrazol-4-yl)-4-oxo-1,4-dihydropyridazin-3-yl]methyl}phenyl)pyrimidin-5-yl]propylcarbamate.

LRMS (ESI) calc'd for C24H26N7O3 [M+H]⁺: 460. Found: 460.

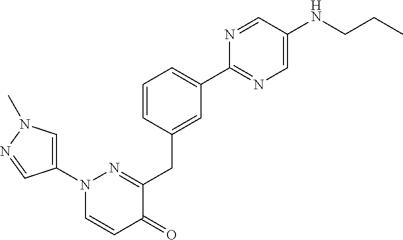

### Step 2. 1-(1-Methyl-1H-pyrazol-4-yl)-3-{3-[5-(propylamino)pyrimidin-2-yl]benzyl}pyridazin-4(1H)-one

To a solution of tert-butyl [2-(3-{[1-(1-methyl-1H-pyrazol-4-yl)-4-oxo-1,4-dihydropyridazin-3-yl]methyl}phenyl)pyrimidin-5-yl]propylcarbamate (131 mg, 0.26 mmol) in DCM (1.3 mL) was added TFA (1.3 mL) and the reaction mixture was stirred at room temperature for 3 hours. The solvent was removed in vacuo, the TFA salt dissolved in DCM-MeOH 4:1, neutralized by filtering through a PL-HCO3 (Stratospheres™, 0.9 mmol) cartridge and the solvent was removed under reduced pressure to provide 1-(1-methyl-1H-pyrazol-4-yl)-3-{3-[5-(propylamino)pyrimidin-2-yl]benzyl}pyridazin-4(1H)-one.

LRMS (ESI) calc'd for C22H24N7O [M+H]⁺: 402. Found: 402.

The following examples were prepared according to Scheme 9 following a similar procedure described for Example #451, which can be achieved by those of ordinary skill in the art of organic synthesis.

| | | | Exact |
|---|---|---|---|
| | | IUPAC | Mass |
| Example | Structure | Name | [M + H]⁺ |
| 452 | 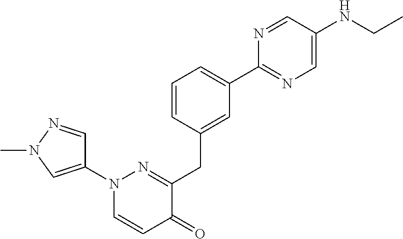 | 3-{3-[5- (ethylamino) pyrimidin-2- yl]benzyl}-1- 1-methyl-1H- pyrazol-4- yl)pyridazin- 4(1H)-one | Calc'd 388, found 338 |
| 453 | 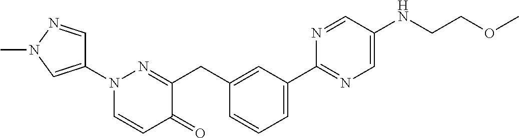 | 3-(3-{5-[(2- methoxyethyl) amino] pyrimidin-2- yl}benzyl)-1- (1-methyl-1H- pyrazol-4- yl)pyridazin- 4(1H)-one | Calc'd 418, found 418 |
| 454 | 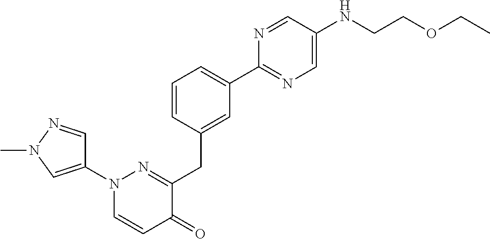 | 3-(3-{5-[(2- ethoxyethyl) amino] pyrimidin-2- yl}benzyl)-1- (1-methyl-1H- pyrazol-4- yl)pyridazin- 4(1H)-one | Calc'd 432, found 432 |
| 455 | 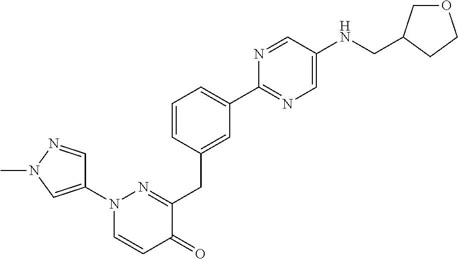 | rac-1-(1- methyl-1H- pyrazol-4-yl)- 3-(3-{5- [(tetrahydro- furan-3- ylmethyl) amino] pyrimidin-2- yl}benzyl) pyridazin- 4(1H)-one | Calc'd 444, found 444 |

### Scheme 9

### Example #456

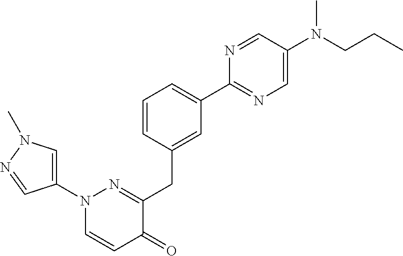

### 3-(3-{5-[Methyl(propyl)amino]pyrimidin-2-yl}benzyl)-1-(1-methyl-1H-pyrazol-4-yl)pyridazin-4(1H)-one

### Step 1. 3-(3-{5-[Methyl(prop)amino]pyrimidin-2-yl}benzyl)-1-(1-methyl-1H-pyrazol-4-yl)pyridazin-4(1-H)-one

To a solution of 1-(1-methyl-1H-pyrazol-4-yl)-3-{3-[5-(propylamino)pyrimidin-2-yl]benzyl}pyridazin-4(1H)-one (Example #451, 25 mg, 0.062 mmol) in MeCN (311 μL) was added aqueous formaldehyde (464 μL, 6.23 mmol); and acetic acid (28 μL, 0.49 mmol) and the temperature was maintained with an external water bath at 20° C. NaBH₃CN (17.6 mg, 0.28 mmol) was added in three portions and the reaction mixture was stirred at room temperature for 4 hours. NaHCO₃ was added followed by the addition of EtOAc, the phases were separated, and the aqueous phase was extracted with EtOAc (3×). The combined organic extracts were dried and the solvent concentrated in vacuo. The product was purified by flash chromatography (MPLC, MeOH-DCM 0-20%) to provide 3-(3-{5-[methyl(propyl)amino]pyrimidin-2-yl}benzyl)-1-(1-methyl-1H-pyrazol-4-yl)pyridazin-4(1H)-one.

LRMS (ESI) calc'd for C23H26N7O [M+H]⁺: 416. Found: 416.

The following example was prepared from Example #453 according to Scheme 9 following a similar procedure described for Example #456, which can be achieved by those of ordinary skill in the art of organic synthesis.

| | | | Exact |
|---|---|---|---|
| | | IUPAC | Mass |
| Example | Structure | Name | [M + H]⁺ |
| 457 | 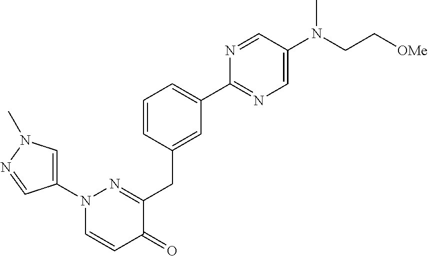 | 3-(3-{5-[(2- methoxyethyl) (methyl)amino] pyrimidin- 2-yl}benzyl)- 1-(1-methyl- 1H-pyrazol-4- yl)pyridazin- 4(1H)-one | Calc'd 432, found 432 |

### Scheme 10

### Example #458

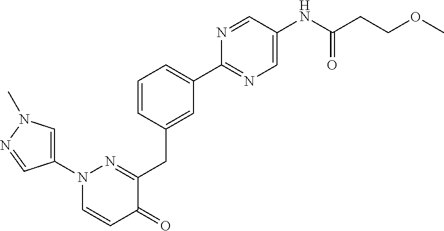

### 3-Methoxy-N-[2-(3-{[1-(1-methyl-1H-pyrazol-4-yl)-4-oxo-1,4-dihydropyridazin-3-yl]methyl}phenyl)pyrimidin-5-yl]propanamide

### Step 1. 3-Methoxy-N-[2-(3-{[1-(1-methyl-1H-pyrazol-4-yl)-4-oxo-1,4-dihydropyridazin-3-yl]methyl}phenyl)pyrimidin-5-yl]propanamide

To a 2 mL microwave vial with stir bar was added 3-[3-(5-aminopyrimidin-2-yl)benzyl]-1-(1-methyl-1H-pyrazol-4-yl)pyridazin-4(1H)-one (Example #183, 30 mg, 0.083 mmol) in DMF (0.5 mL) followed by 3-methoxypropanoic acid (8.6 mg, 0.083 mmol), DIPEA (0.029 mL, 0.167 mmol) and 1-propanephosphonic acid cyclic anhydride (0.068 mL, 0.100 mmol). The reaction was stirred at 0° C. for 20 minutes then at room temperature for 48 hours. The reaction mixture was filtered, then purified by reverse-phase mass-triggered preparative HPLC to provide 3-methoxy-N-[2-(3-{[1-(1-methyl-1H-pyrazol-4-yl)-4-oxo-1,4-dihydropyridazin-3-yl]methyl}phenyl)pyrimidin-5-yl]propanamide.

LRMS calc'd for C23H24N7O3 [M+H]⁺: 446. Found: 446.

The following examples were prepared according to Scheme 10 following a similar procedure described for Example #458, which can be achieved by those of ordinary skill in the art of organic synthesis.

| | | | Exact |
|---|---|---|---|
| | | IUPAC | Mass |
| Example | Structure | Name | [M + H]⁺ |
| 459 | 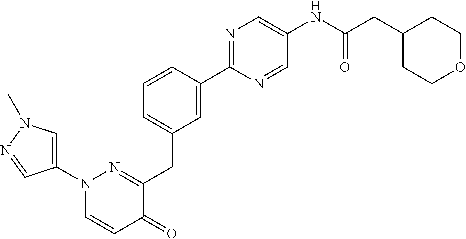 | N-[2-(3-{[1- (1-methyl-1H- pyrazol-4-yl)- 4-oxo-1,4- dihydro- pyridazin-3- yl]methyl} phenyl) pyrimidin- 5-yl]-2- (tetrahydro 2H-pyran-4- yl)acetamide | Calc'd 486, found 486 |
| 460 | 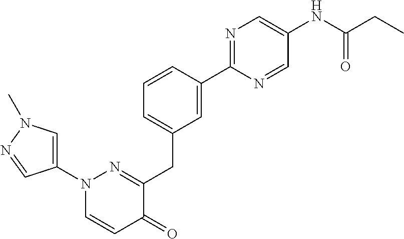 | N-[2-(3-{[1- (1-methyl-1H- pyrazol-4-yl)- 4-oxo-1,4- dihydro- pyridazin-3- yl]methyl} phenyl) pyrimidin- 5-yl} propanamide | Calc'd 416, found 416 |
| 461 | 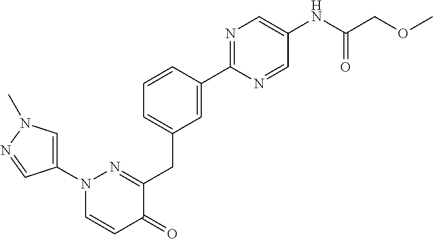 | 2-methoxy-N- [2-(3-{[1-(1- methyl-1H- pyrazol-4-yl)- 4-oxo-1,4- dihydro- pyridazin-3- yl]methyl} phenyl) pyrimidin-5- yl]acetamide | Calc'd 432, found 432 |
| 462 | 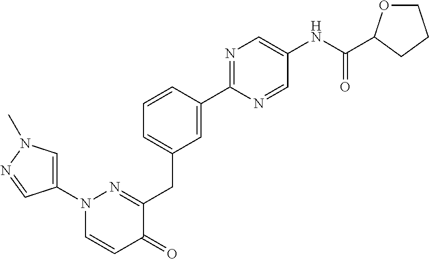 | rac-N-[2-(3- {[1-(1- methyl-1H- pyrazol-4-yl)- 4-oxo-1,4- dihydro- pyridazin-3- yl}methyl} phenyl) pyrimidin- 5-yl]tetra- hydrofuran-2- carboxamide | Calc'd 458, found 458 |
| 463 | 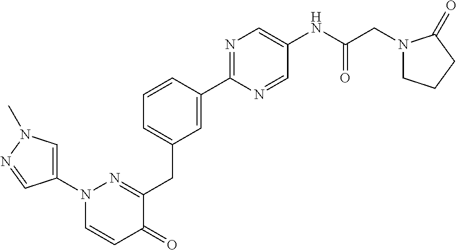 | N-[2-(3-{[1- (1-methyl-1H- pyrazol-4-yl)- 4-oxo-1,4- dihydro- pyridazin-3- yl]methyl} phenyl) pyrimidin- 5-yl]-2-(2- oxopyrrolidin- 1- yl)acetamide | Calc'd 485, found 485 |
| 464 | 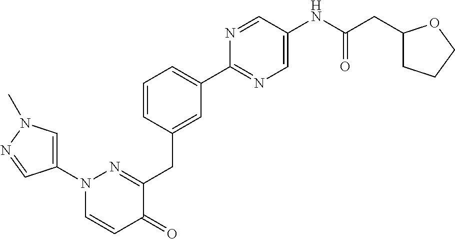 | rac-N-[2-(3- {[1-(1- methyl-1H- pyrazol-4-yl)- 4-oxo-1,4- dihydro- pyridazin-3- yl]methyl} phenyl) pyrimidin- 5-yl]-2- (tetrahydro- furan-2- yl)acetamide | Calc'd 472, found 472 |

### Scheme 10

### Example #465

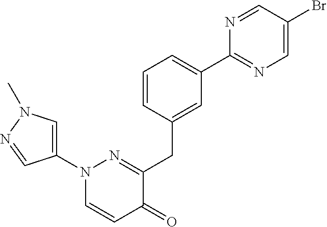

### 3-[3-(5-Bromopyrimidin-2-yl)benzyl]-1-(1-methyl-1H-pyrazol-4-yl)pyridazin-4(1H)-one

### Step 1. 3-[3-(5-Bromopyrimidin-2-yl)benzyl]-1-(1-methyl-1H-pyrazol-4-yl)pyridazin-4(1H)-one

To a solution of 3-[3-(5-aminopyrimidin-2-yl)benzyl]-1-(1-methyl-1H-pyrazol-4-yl)pyridazin-4(1H)-one (Example #183, 2.40 g, 6.67 mmol) and CuBr (5.27 g, 36.7 mmol) in dry acetonitrile (51 mL) was added tert-butyl nitrite (3.44 g, 33.4 mmol) under Ar. The reaction mixture was stirred for 16 hr at room temperature. Water was added, followed by the addition of aqueous ammonia (100 mL). The mixture was extracted with EtOAc (4×). The combined organic extracts were dried over Na₂SO₄, filtered and concentrated in vacuo. Purification of the residue by flash chromatography (MPLC, 0-20% MeOH-DCM) gave 3-[3-(5-bromopyrimidin-2-yl)benzyl]-1-(1-methyl-1H-pyrazol-4-yl)pyridazin-4(1H)-one.

LRMS (ESI) calc'd for C19H16BrN6O [M+H]⁺: 423. Found: 423.

### Scheme 10

### Example #466

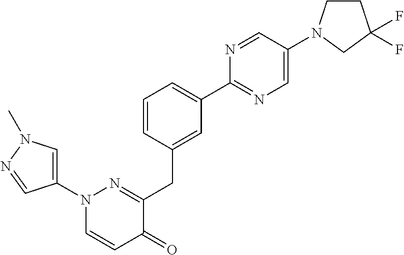

### 3-{3-[5-(3,3-Difluoropyrrolidin-1-yl)pyrimidin-2-yl]benzyl}-1-(1-methyl-1H-pyrazol-4-yl)pyridazin-4(1H)-one

### Step 1. 3-{3-[5-(3,3-Difluoropyrrolidin-1-yl)pyrimidin-2-yl]benzyl}-1-(1-methyl-1H-pyrazol-4-yl)pyridazin-4(1H)-one

A microwave vial was charged with 3-[3-(5-bromopyrimidin-2-yl)benzyl]-1-(1-methyl-1H-pyrazol-4-yl)pyridazin-4(1H)-one (Example #465, 50 mg, 0.12 mmol), DavePhos (9.3 mg, 0.024 mmol), Pd₂(dba)₃ (10.8 mg, 0.012 mmol), NaOt-Bu (28.4 mg, 0.29 mmol) and 3,3-difluoropyrrolidine (33.9 mg, 0.23 mmol). Toluene (1.2 mL) was added and the reaction mixture was degassed by bubbling through N₂. After keeping it at 85° C. for 36 hours the reaction mixture was filtered through Celite, eluting with 4:1 DCM-MeOH. The filtrate was concentrated in vacuo and the residue purified by mass directed reverse phase preparative HPLC. The product fractions were filtered through MP-HCO₃ (Stratospheres™, 0.9 mmol) column to remove the TFA and the solvent removed by centrifugation to provide 3-{3-[5-(3,3-difluoropyrrolidin-1-yl)pyrimidin-2-yl]benzyl}-1-(1-methyl-1H-pyrazol-4-yl)pyridazin-4(1H)-one.

LRMS (ESI) calc'd for C23H22F2N7O [M+H]⁺: 450. Found: 450.

The following examples were prepared according to Scheme 10 following a similar procedure described for Example #466, which can be achieved by those of ordinary skill in the art of Exact

| | | | Exact |
|---|---|---|---|
| | | IUPAC | Mass |
| Example | Structure | Name | [M + H]⁺ |
| 467 | 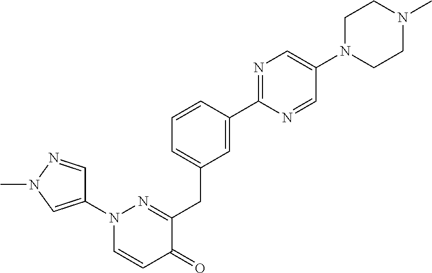 | 3-{3-[5-(4- methyl- piperazin-1- yl)pyrimidin- 2-yl]benzyl}- 1-(1-methyl- 1H-pyrazol-4- yl)pyridazin- 4(1H)-one | Calc'd 443, found 443 |
| 468 | 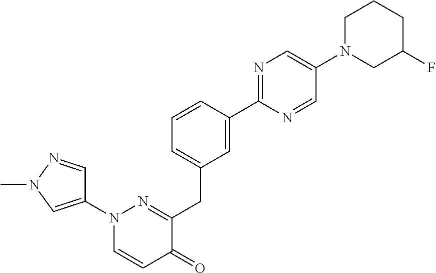 | rac-3-{3-[5- (3-fluoro- piperidin-1- yl)pyrimidin- 2-yl]benzyl)- 1-(1-methyl- 1H-pyrazol-4- yl)pyridazin- 4(1H)-one | Calc'd 446, found 446 |
| 469 | 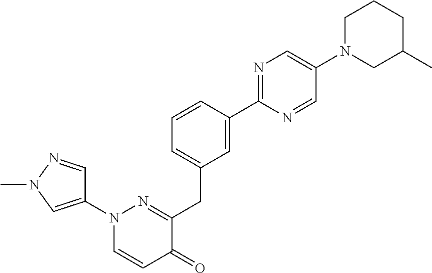 | rac-3-{3-[5- (3-methyl- piperidin-1- yl)pyrimidin- 2-yl]benzyl}- 1-(1-methyl- 1H-pyrazol-4- yl)pyridazin- 4(1H)-one | Calc'd 442, found 442 |
| 470 | 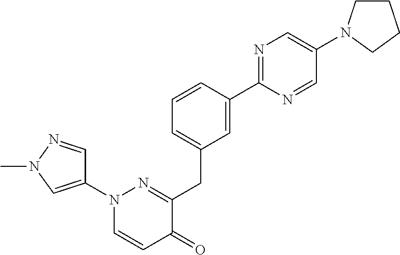 | 1-(1-methyl- 1H-pyrazol-4- yl)-3-[3-(5- pyrrolidin-1- ylpyrimidin- 2-yl)benzyl] pyridazin- 4(1H)-one | Calc'd 414, found 414 |
| 471 | 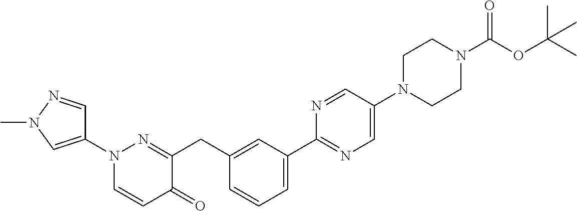 | tert-butyl 4- [2-(3-{[1-(1- methyl-1H- pyrazol-4-yl)- 4-oxo-1,4- dihydro- pyridazin-3- yl]methyl} phenyl) pyrimidin-5- yl]piperazine- 1-carboxylate | Calc'd 529, found 529 |
| 472 | 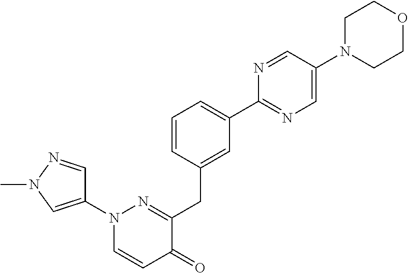 | 1-(1-methyl- 1H-pyrazol-4- yl)-3-[3-(5- morpholin-4- ylpyrimidin- 2-yl)benzyl] pyridazin- 4(1H)-one | Calc'd 430, found 430 |

### Scheme 10

### Example #473

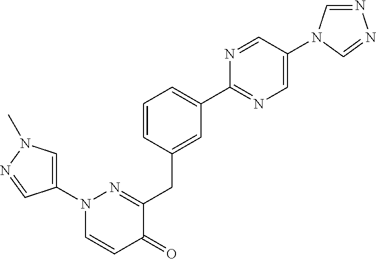

### 1-(1-Methyl-1H-pyrazol-4-yl)-3-{3-[5-(4H-1,2,4-triazol-4-yl)pyrimidin-2-yl]benzyl}pyridazin-4(1H)-one

### Step 1. 1-(1-Methyl-1H-pyrazol-4-yl)-3-{3-[5-(4H-1,2,4-triazol-4-yl)pyrimidin-2-yl]benzyl}pyridazin-4(1H)-one

To a suspension of 3-[3-(5-aminopyrimidin-2-yl)benzyl]-1-(1-methyl-1H-pyrazol-4-yl)pyridazin-4(1H)-one (Example #183, 40 mg, 0.11 mmol) in toluene (445 μL) was added N′-[(E)-(dimethylamino)methylidene]-N,N-dimethylhydrazonoformamide (47 mg, 0.33 mmol) and PTSA (3.8 mg, 0.022 mmol). The reaction mixture was heated to 100° C. for 16 hours. Subsequently, the reaction mixture was washed with saturated NaHCO₃, the organic phase was dried over Na₂SO₄ and the solvent was concentrated in vacuo. The residue was purified by flash chromatography (MPLC, 0-20% MeOH-DCM) to provide 1-(1-methyl-1H-pyrazol-4-yl)-3-{3-[5-(4H-1,2,4-triazol-4-yl)pyrimidin-2-yl]benzyl}pyridazin-4(1H)-one.

LRMS (ESI) calc'd for C21H18N9O [M+H]⁺: 412. Found: 412.

### Scheme 11

### Example #474

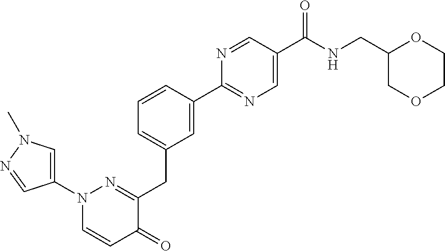

### rac-N-(1,4-Dioxan-2-ylmethyl)-2-(3-{[1-(1-methyl-1H-pyrazol-4-yl)-4-oxo-1,4-dihydropyridazin-3-yl]methyl}phenyl)pyrimidine-5-carboxamide

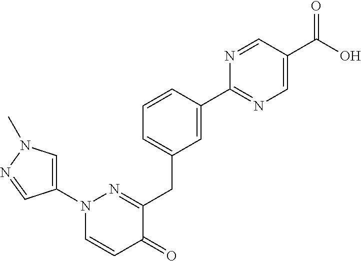

### Step 1. 2-(3-{[1-(1-Methyl-1H-pyrazol-4-yl)-4-oxo-1,4-dihydropyridazin-3-yl]methyl}phenyl)pyrimidine-5-carboxylic acid

Methyl 2-(3-{[1-(1-methyl-1 H-pyrazol-4-yl)-4-oxo-1,4-dihydropyridazin-3-yl]methyl}phenyl)pyrimidine-5-carboxylate (Example #15, 600 mg, 1.49 mmol) was suspended in methanol (2.5 mL) in a 5 mL microwave vial and 2 N sodium hydroxide (3.73 mL, 7.46 mmol) was added. The vial was sealed and heated to 100° C. for 10 minutes in the microwave. On completion of the reaction, the reaction mixture was transferred to an Erlenmeyer flask with a small-amount of water and acidified until solid crashed out of solution (3-4 mL of 2 N HCl). The solution was then diluted with 100 mL of water and extracted with 3:1 chloroform:IPA (2×200 mL). The organic layer was dried over Na₂SO₄, filtered and concentrated in vacuo to give 2-(3-{[1-(1-Methyl-1H-pyrazol-4-yl)-4-oxo-1,4-dihydropyridazin-3-yl]methyl}phenyl)pyrimidine-5-carboxylic acid.

LRMS calc'd for C20H16N6O3 [M+H]⁺: 389. Found: 390.

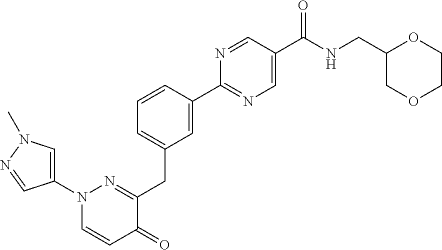

### Step 2. rac-N-(1,4-Dioxan-2-ylmethyl)-2-(3-{[1-(1-methyl-1H-pyrazol-4-yl)-4-oxo-1,4-dihydropyridazin-3-yl]methyl}phenyl)pyrimidine-5-carboxamide

To a 2 mL microwave vial with stir bar was added Si-Carbodiimide (257 mg, 0.278 mmol) followed by HOBt (21.3 mg, 0.139 mmol), and 1-(1,4-dioxan-2-yl)methanamine (41 mg, 0.35 mmol). 2-(3-{[1-(1-methyl-1H-pyrazol-4-yl)-4-oxo-1,4-dihydropyridazin-3-yl]methyl}phenyl)pyrimidine-5-carboxylic acid (27 mg, 0.070 mmol) was added as a solution in DMF (750 L) followed by the addition of DIPEA (61 μL, 0.35 mmol). The reaction was heated to 100° C. for 10 minutes under microwave irradiation (Biotage, Initiator). To remove HOBt, Si-Carbonate (294 mg, 0.209 mmol) was added to the vial followed by additional DMF (500 μL). The reaction was heated to 100° C. for 5 minutes in the microwave. The reaction mixture was filtered and the solvent removed in vacuo. The residue was purified by reverse-phase mass-directed preparative HPLC to give rac-N-(1,4-dioxan-2-ylmethyl)-2-(3-{[1-(1-methyl-1H-pyrazol-4-yl)-4-oxo-1,4-dihydropyridazin-3-yl]methyl}phenyl)pyrimidine-5-carboxamide.

LRMS calc'd for C25H26N7O4 [M+H]⁺: 48-8. Found: 488.

The following examples were prepared according to Scheme 11 following a similar procedure described for Example #474 Step 2, which can be achieved by those of ordinary skill in the art of organic synthesis.

| | | | Exact |
|---|---|---|---|
| | | IUPAC | Mass |
| Example | Structure | Name | [M + H] |
| 475 | 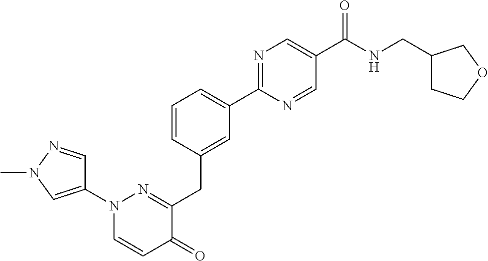 | rac-2-(3-{[1- (1-methyl-1H- pyrazol-4-yl) 4-oxo-1,4- dihydro- pyridazin-3- yl]methyl} phenyl)-N- (tetrahydro- furan-3- ylmethyl) pyrimidine-5- carboxamide | Calc'd 472, found 472 |
| 476 | 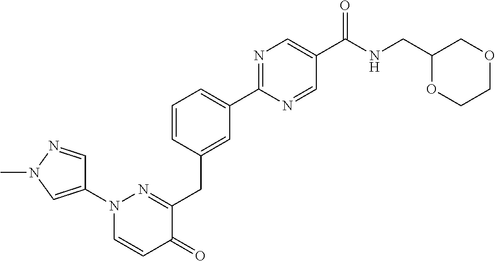 | rac-N-(1,4- dioxan-2- ylmethyl)-2- (3-{[1-(1- methyl-1H- pyrazol-4-yl)- 4-oxo-1,4- dihydro- pyridazin-3- yl]methyl} phenyl) pyrimidine-5- carboxamide | Calc'd 488, found 488 |
| 477 | 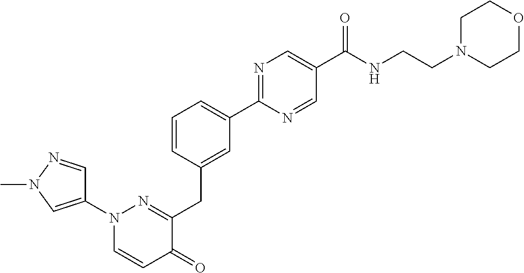 | 2-(3-{[1-(1- methyl-1H- pyrazol-4-yl)- 4-oxo-1,4- dihydro- pyridazin-3- yl]methyl} phenyl)-N-(2- morpholin-4- ylethyl) pyrimidine-5- carboxamide | Calc'd 501, found 501 |
| 478 | 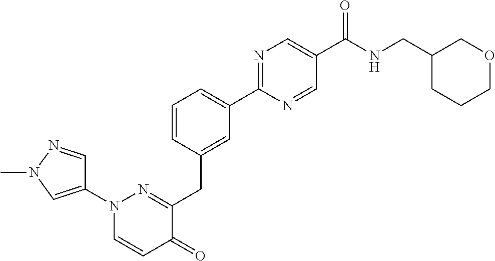 | rac-2-(3-{[1- (1-methyl-1H- pyrazol-4-yl)- 4-oxo-1,4- dihydro- pyridazin-3- yl]methyl} phenyl)-N- tetrahydro- 2H-pyran-3- ylmethyl) pyrimidine-5- carboxamide | Calc'd 486, found 486 |
| 479 | 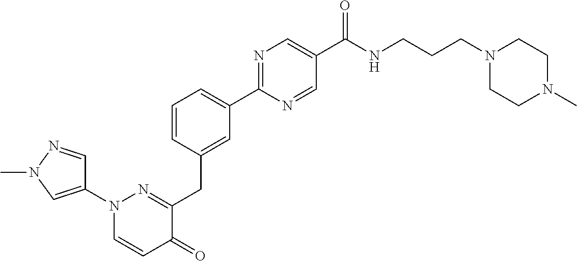 | N-[3-(4- methyl- piperazin-1- yl)propyl]-2- (3-{[1-(1- methyl-1H- pyrazol-4-yl)- 4-oxo-1,4- dihydro- pyridazin-3- yl]methyl} phenyl) pyrimidine-5- carboxamide | Calc'd 528, found 528 |
| 480 | 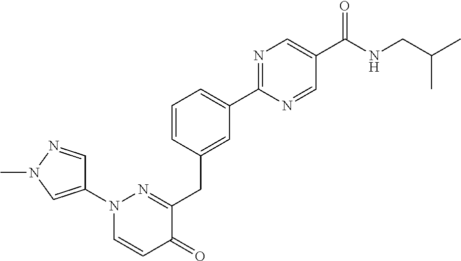 | N-(2- methylpropyl)- 2-(3-{[1-(1- methyl-1H- pyrazol-4-yl)- 4-oxo-1,4- dihydro- pyridazin-3- yl]methyl} phenyl) pyrimidine-5- carboxamide | Calc'd 444, found 444 |
| 481 | 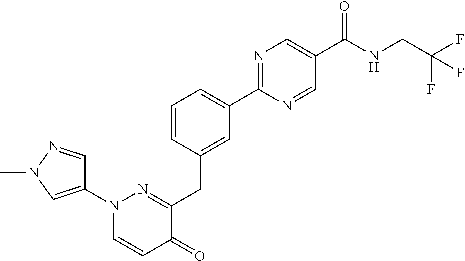 | 2-(3-{[1-(1- methyl-1H- pyrazol-4-yl)- 4-oxo-1,4- dihydro- pyridazin-3- yl]methyl} phenyl)-N-(2,2,2- trifluoroethyl) pyrimidine-5- carboxamide | Calc'd 470, found 470 |
| 482 | 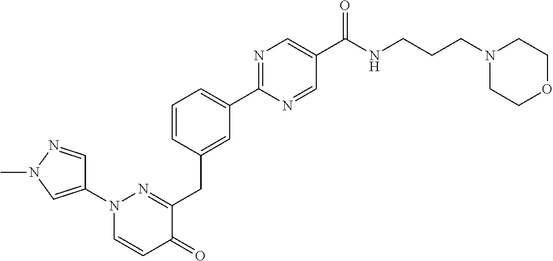 | 2-(3-{[1-(1- methyl-1H- pyrazol-4-yl)- 4-oxo-1,4- dihydro- pyridazin-3- yl]methyl} phenyl)-N-(3- morpholin-4- ylpropyl) pyrimidine-5- carboxamide | Calc'd 515, found 515 |
| 483 | 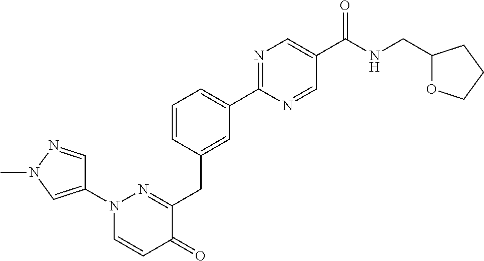 | rac-2-(3-{[1- (1-methyl-1H- pyrazol-4-yl)- 4-oxo-1,4- dihydro- pyridazin-3- yl]methyl} phenyl)-N- (tetrahydro- furan-2- ylmethyl) pyrimidine-5- carboxamide | Calc'd 472, found 472 |
| 484 | 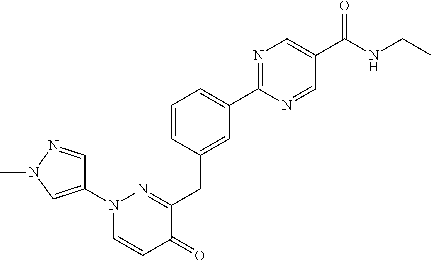 | N-ethyl-2-(3- {[1-(1- methyl-1H- pyrazol-4-yl)- 4-oxo-1,4- dihydro- pyridazin-3- yl]methyl} phenyl) pyrimidine-5- carboxamide | Calc'd 416, found 416 |
| 485 | 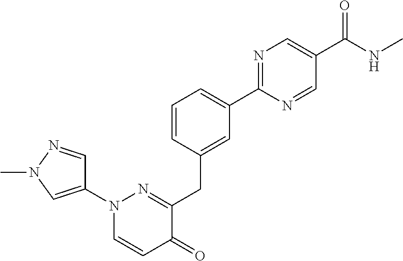 | N-methyl-2- (3-{[1-(1- methyl-1H- pyrazol-4-yl)- 4-oxo-1,4- dihydro- pyridazin-3- yl]methyl} phenyl) pyrimidine-5- carboxamide | Calc'd 402, found 402 |

### Scheme 12

### Alternative Aniline Synthesis

### Intermediate #132

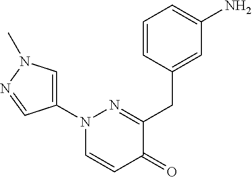

### 3-(3-Aminobenzyl)-1-(1-methyl-1H-pyrazol-4-yl)pyridazin-4(1H)-one

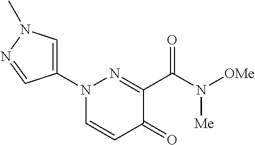

### Step 1. N-Methoxy-N-methyl-1-(1-methyl-1H-pyrazol-4-yl)-4-oxo-1,4-dihydropyridazine-3-carboxamide

1-(1-Methyl-1H-pyrazol-4-yl)-4-oxo-1,4-dihydropyridazine-3-carboxylic acid (Intermediate #1 Step 3, 3.60 g, 16.4 mmol), N,O-dimethylhydroxylamine hydrochloride (3.19 g, 32.7 mmol), DIPEA (11.4 mL, 65.4 mmol), EDC (4.70 g, 24.5 mmol) and HOBt (3.76 g, 24.5 mmol) were stirred in DMF (80 mL) at r.t. overnight. The solvent was removed in vacuo while loading onto silica. The crude residue was purified by flash chromatography (MPLC, 0-15% MeOH-DCM) and the product fractions combined and washed with saturated NaHCO₃ to give N-methoxy-N-methyl-1-(1-methyl-1H-pyrazol-4-yl)-4-oxo-1,4-dihydropyridazine-3-carboxamide as a red solid.

LRMS (ESI) calc'd for C11H14N5O3 [M+H]⁺: 264. Found: 264.

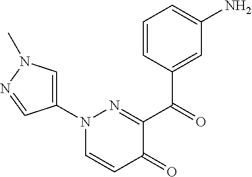

### Step 2. 3-(3-aminobenzoyl)-1-(1-methyl-1H-pyrazol-4-yl)pyridazin-4(1H)-one

N-Methoxy-N-methyl-1-(1-methyl-1H-pyrazol-4-yl)-4-oxo-1,4-dihydropyridazine-3-carboxamide (2.46 g, 9.34 mmol) was taken up in THF (110 mL) and cooled to −78° C. {3-[Bis(trimethylsilyl)amino]phenyl}magnesium chloride (23.3 mL, 23.3 mmol) was added and stirring at −78° C. continued for 45 minutes. 2 N HCl (24 mL) was added and the resulting solution was allowed to warm to ambient temperature and maintained for 30 additional minutes. The aqueous mixture was neutralized with solid Na₂CO₃ and the products extracted into MeOH-DCM (˜4 L). The combined organic extracts were dried over MgSO₄, filtered, and concentrated in vacuo. The crude residue was triturated in DCM to give 3-(3-aminobenzoyl)-1-(1-methyl-1H-pyrazol-4-yl)pyridazin-4(1H)-one as a yellow solid.

LRMS (ESI) calc'd for C15H14N5O2 [M+H]⁺: 296. Found: 296.

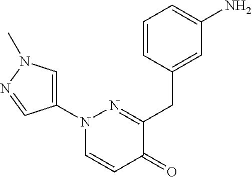

### Step 3. 3-(3-Aminobenzyl)-1-(1-methyl-1H-pyrazol-4-yl)pyridazin-4(1H)-one

3-(3-Aminobenzoyl)-1-(1-methyl-1H-pyrazol-4-yl)pyridazin-4(1H)-one (2.35 g, 7.96 mmol) and Pd(OH)₂/C (10 wt %, 0.279 g, 0.398 mmol) were stirred in MeOH (80 mL), under H₂ (1 atm) at 50° C. overnight. Additional Pd(OH)₂/C (10 wt %, 0.279 g, 0.398 mmol) was added and stirring under H₂ (1 atm) at 50° C. continued overnight. The catalyst was removed by filtration through Celite and the solvent removed in vacuo. The residue was triturated in DCM to give the desired product. The filtrate was purified directly by flash chromatography (MPLC, 0-10% MeOH-DCM) to give additional product after triturating in DCM. The Celite used to filter off the catalyst was washed with DMF (˜100 mL), the solvent was removed in vacuo while loading onto silica and purification of the residue by flash chromatography (MPLC, 0-10% MeOH-DCM) gave 3-(3-aminobenzyl)-1-(1-methyl-1H-pyrazol-4-yl)pyridazin-4(1H)-one as a white solid.

LRMS (ESI) calc'd for C15H15N5O [M+H]⁺: 282. Found: 282.

The following intermediate was prepared from 3-(3-aminobenzoyl)-1-(4-chlorophenyl)pyridazin-4(1H)-one (Scheme 14, Example #640 Step 2) according to the Alternative Aniline Synthesis following similar procedures described for Intermediate #132, which can be achieved by those of ordinary skill in the art of organic synthesis.

| | | | Exact Mass |
|---|---|---|---|
| Intermediate | Structure | IUPAC Name | [M + H]⁺ |
| 157 | 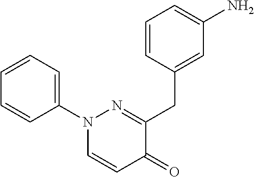 | 3-(3-aminobenzyl)- 1-phenylpyridazin- 4(1H)-one | Calc'd 278, found 278 |

### Scheme 12

### Example #486

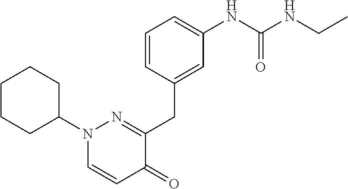

### 1-Ethyl-3-{3-[(4-oxo-1-phenyl-1,4-dihydropyridazin-3-yl)methyl]phenyl}urea

### Step 1. T-Ethyl-3-{3-[(4-oxo-1-phenyl-1,4-dihydropyridazin-3-yl)methyl]phenyl}urea

3-(3-Aminobenzyl)-1-phenylpyridazin-4(1H)-one (Intermediate #157, 50 mg, 0.18 mmol) and ethyl isocyanate (0.016 mL, 0.20 mmol) were dissolved in THF (1.5 mL) and the mixture was stirred at r.t. for 36 hrs. DCM was added and the reaction mixture was washed sequentially with sat. NaHCO₃ solution and brine. The organic phase was dried over Na₂SO₄, filtered, concentrated, and the residue was purified by reverse phase preparative HPLC (0-100% MeCN—H₂O; 0.05% TFA) to afford 1-ethyl-3-{3-[(4-oxo-1-phenyl-1,4-dihydropyridazin-3-yl)methyl]phenyl}urea after removal of the solvent in vacuo.,

LRMS (ESI) calc'd for C20H21N4O2 [M+H]⁺: 349. Found: 349.

### Scheme 12

### Example #487

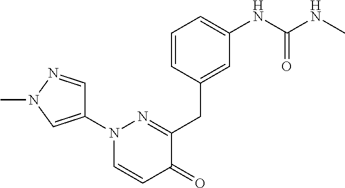

### 1-Methyl-3-(3-{[1-(1-methyl-1H-pyrazol-4-yl)-4-oxo-1,4-dihydropyridazin-3-yl]methyl}phenyl)urea

### Step 1. 1-Methyl-3-(3-{[1-(1-methyl-1H-pyrazol-4-yl)-4-oxo-1,4-dihydropyridazin-3-yl]methyl}phenylurea

A 2 mL microwave vial was charged with acetic acid (18 μL, 0.31 mmol), THF (1 mL) and DIPEA (0.099 mL, 0.57 mmol). Diphenylphosphoryl azide (90 mg, 0.33 mmol) was added and the reaction was stirred at r.t. for 4 hr. 3-(3-Aminobenzyl)-1-(1-methyl-1H-pyrazol-4-yl)pyridazin-4(1H)-one (Intermediate #132, 40 mg, 0.14 mmol) was then added, the vial was sealed and heated to 90° C. overnight. The reaction mixture was concentrated, redissolved in DMSO (1 mL) and purified by preparative HPLC. Fractions containing pure compound were collected, the solvent was removed in vacuo. The residue was dissolved in DCM and transferred to a Bohdan block containing MP-Carbonate. DIPEA (5 μL) was added and the vessel was shaken for 4 hrs at room temperature, filtered, and concentrated. The residues were then dissolved in MeOH and water, frozen, and lyophilized to provide 1-methyl-3-(3-{[1-(1-methyl-1H-pyrazol-4-yl)-4-oxo-1,4-dihydropyridazin-3-yl]methyl}phenyl)urea.

LRMS (ESI) calc'd for C17H19N6O2 [M+H]⁺: 339. Found: 339.

The following examples were prepared from Intermediates #132 and 134 according to Scheme 12 following similar procedures described for Examples #486 and 487, which can be achieved by those of ordinary skill in the art of organic synthesis.

| | | | Exact |
|---|---|---|---|
| | | | Mass |
| Example | Structure | IUPAC Name | [M + H]⁺ |
| 488 | 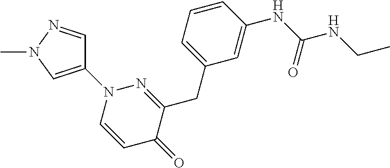 | 1-ethyl-3-(3- {[1-(1-methyl- 1H-pyrazol-4- yl)-4-oxo-1,4- dihydropyridazin- 3-yl]methyl} phenyl)urea | Calc'd 353, found 353 |
| 489 | 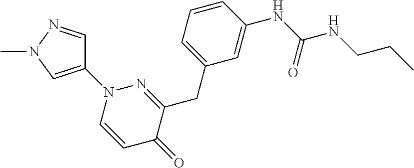 | 1-(3-{[1-(1- methyl-1H- pyrazol-4-yl)-4- oxo-1,4- dihydropyridazin- 3-yl]methyl} phenyl)-3- propylurea | Calc'd 367, found 367 |
| 490 | 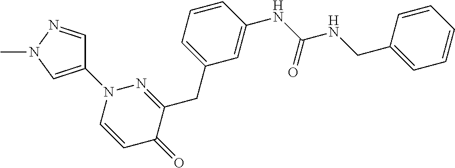 | 1-benzyl-3-(3- {[1-(1-methyl- 1H-pyrazol-4- yl)-4-oxo-1,4- dihydropyridazin- 3-yl]methyl} phenyl)urea | Calc'd 415, found 415 |
| 491 | 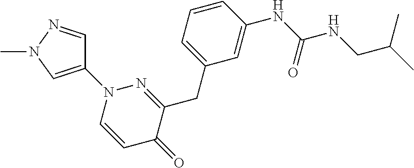 | 1-(2- methylpropyl)- 3-(3-{[1-(1- methyl-1H- pyrazol-4-yl)-4- oxo-1,4- dihydropyridazin- 3-yl]methyl} phenyl)urea | Calc'd 381, found 381 |
| 492 | 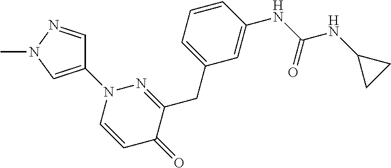 | 1-cyclopropyl- 3-(3-{[1-(1- methyl-1H- pyrazol-4-yl)-4- oxo-1,4- dihydropyridazin- 3-yl]methyl} phenyl)urea | Calc'd 365, found 365 |
| 493 | 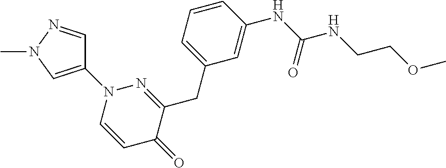 | 1-(2- methoxyethyl)- 3-(3-{[1-(1- methyl-1H- pyrazol-4-yl)-4- oxo-1,4- dihydropyridazin- 3-yl]methyl} phenyl)urea | Calc'd 383, found 383 |
| 494 | 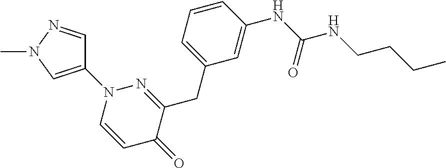 | 1-butyl-3-(3- {[1-(1-methyl- 1H-pyrazol-4- yl)-4-oxo-1,4- dihydropyridazin- 3-yl]methyl} phenyl)urea | Calc'd 381, found 381 |
| 495 | 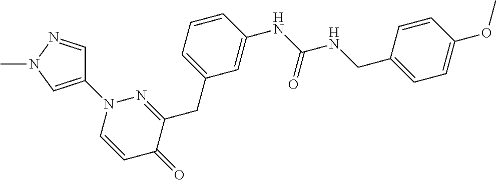 | 1-(4- methoxybenzyl)- 3-(3-{[1-(1- methyl-1H- pyrazol-4-yl)-4- oxo-1,4- dihydropyridazin- 3-yl]methyl} phenyl)urea | Calc'd 445, found 445 |
| 496 | 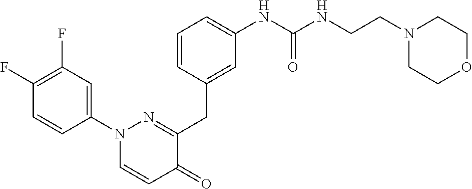 | 1-(3-{[1-(3,4- difluorophenyl)- 4-oxo-1,4- dihydropyridazin- 3-yl]methyl} phenyl)-3-(2- morpholin-4- ylethyl)urea | Calc'd 470, found 470 |

### Scheme 12

### Example #497

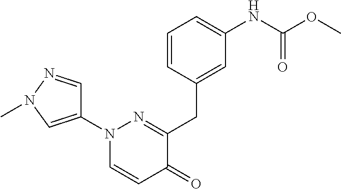

### Methyl(3-{[1-(1-methyl-1H-pyrazol-4-yl)-4-oxo-1,4-dihydropyridazin-3-yl]methyl}phenyl)carbamate

### Step 1. Methyl(3-{[1-(1-methyl-1H-pyrazol-4-yl)-4-oxo-1,4-dihydropyridazin-3-yl]methyl}phenyl)carbamate

PS-DIPEA (109 mg, 0.427 mmol) was added to a Bohdan vessel followed by 3-(3-aminobenzyl)-1-(1-methyl-1H-pyrazol-4-yl)pyridazin-4(1H)-one (Intermediate #132, 40 mg, 0.14 mmol) suspended in THF (1 mL). Methyl chloroformate (13 μL, 0.17 mmol) was then added and the vial was capped and shaken at r.t. overnight. DCM (1 mL) was added followed by PS-DIPEA (0.14 mmol, 36 mg) and methyl chloroformate (0.071 mmol). The reaction mixture was shaken for 24 hrs. Upon completion, additional DCM (1 mL) was added followed by MP-trisamine (210 mg, 0.43 mmol) to scavenge excess methyl chloroformate. The mixture was stirred for 3 hrs. The solvent was removed and the residue was purified by reverse phase preparative HPLC to give methyl(3-{[1-(1-methyl-1H-pyrazol-4-yl)-4-oxo-1,4-dihydropyridazin-3-yl]methyl}phenyl)carbamate.

LRMS (ESI) calc'd for C17H17N5O3 [M+H]⁺: 340. Found: 340.

The following examples were prepared from Intermediates #132-138 according to Scheme 12 following a similar procedure described for Example #497, which can be achieved by those of ordinary skill in the art of organic synthesis.

| | | | Exact |
|---|---|---|---|
| | | | Mass |
| Example | Structure | IUPAC Name | [M + H]+ |
| 498 | 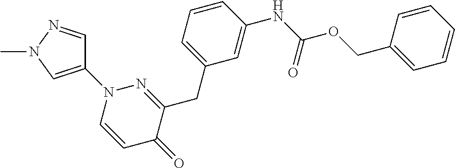 | benzyl(3-{[1- (1-methyl-1H- pyrazol-4-yl)-4- oxo-1,4- dihydropyridazin- 3-yl]methyl} phenyl)carbamate | Calc'd 416, found 416 |
| 499 | 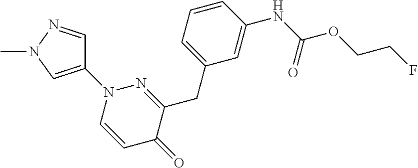 | 2-fluoroethyl (3-{[1-(1- methyl-1H- pyrazol-4-yl)-4- oxo-1,4- dihydropyridazin- 3-yl]methyl} phenyl)carbamate | Calc'd 372, found 372 |
| 500 | 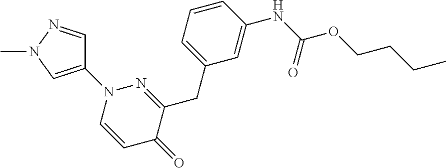 | Butyl (3-{[1-(1- methyl-1H- pyrazol-4-yl)-4- oxo-1,4- dihydropyridazin- 3-yl]methyl} phenyl)carbamate | Calc'd 382, found 382 |
| 501 | 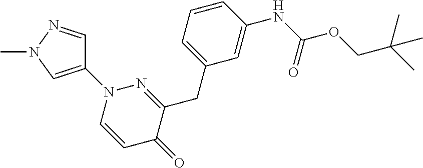 | 2,2- dimethylpropyl (3-{[1-(1- methyl-1H- pyrazol-4-yl)-4- oxo-1,4- dihydropyridazin- 3-yl]methyl} phenyl)carbamate | Calc'd 396, found 396 |
| 502 | 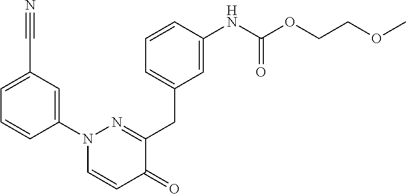 | 2-methoxyethyl (3-{[1-(3- cyanophenyl)-4- oxo-1,4- dihydropyridazin- 3-yl]methyl} phenyl)carbamate | Calc'd 405, found 405 |
| 503 | 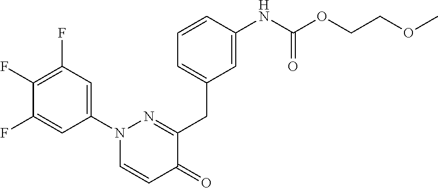 | 2-methoxyethyl (3-{[4-oxo-1- (3,4,5- trifluorophenyl)- 1,4- dihydropyridazin- 3-yl]methyl} phenyl)carbamate | Calc'd 434, found 434 |
| 504 | 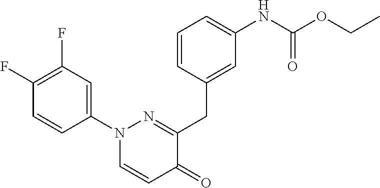 | ethyl (3-{[1- (3,4- difluorophenyl)- 4-oxo-1,4- dihydropyridazin- 3-yl]methyl} phenyl)carbamate | Calc'd 386, found 386 |
| 505 | 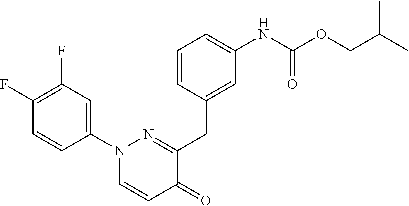 | 2-methylpropyl (3-{[1-(3,4- difluorophenyl)- 4-oxo-1,4- dihydropyridazin- 3-yl]methyl} phenyl)carbamate | Calc'd 414, found 414 |
| 506 | 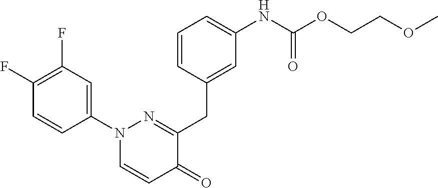 | 2-methoxyethyl (3-{[1-(3,4- difluorophenyl)- 4-oxo-1,4- dihydropyridazin- 3-yl]methyl} phenyl)carbamate | Calc'd 416, found 416 |
| 507 | 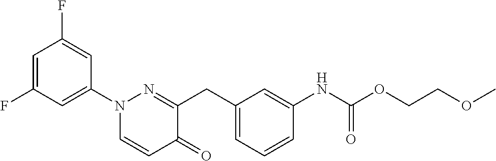 | 2-methoxyethyl (3-{[1-(3,5- difluorophenyl)- 4-oxo-1,4- dihydropyridazin- 3-yl]methyl} phenyl)carbamate | Calc'd 416, found 416 |
| 508 | 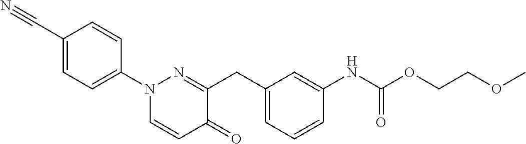 | 2-methoxyethyl (3-{[1-(4- cyanophenyl)-4- oxo-1,4- dihydropyridazin- 3-yl]methyl} phenyl)carbamate | Calc'd 405, found 405 |
| 509 | 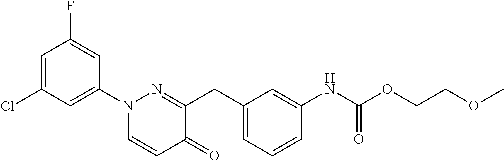 | 2-methoxyethyl (3-{[1-(3- chloro-5- fluorophenyl)- 4-oxo-1,4- dihydropyridazin- 3-yl]methyl} phenyl)carbamate | Calc'd 432, found 432 |

### Scheme 12

### Example #510

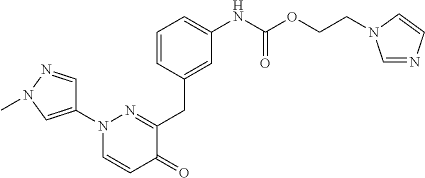

### 2-(1H-Imidazol-1-yl)ethyl(3-{[1-(1-methyl-1H-pyrazol-4-yl)-4-oxo-1,4-dihydropyridazin-3-yl]methyl}phenyl)carbamate

### Step 1. 2-(1H-Imidazol-1-yl)ethyl(3-{[1-(1-methyl-1H-pyrazol-4-yl)-4-oxo-1,4-dihydropyridazin-3-yl]methyl}phenyl)carbamate

A stock solution of N,N′-disuccinimidyl carbonate was prepared by dissolving N,N-disuccinimidyl carbonate (73 mg, 0.28 mmol) in MeCN (500 μL) and adding triethylamine (79 μL, 0.57 mmol). To a solution of 2-(1H-imidazol-1-yl)ethanol (32 mg, 0.28 mmol) in MeCN (100 μL) was added 0.5 mL of N,N-disuccinimidyl carbonate stock solution. The reaction was stirred at r.t. overnight. Subsequently a solution of 3-(3-aminobenzyl)-1-(1-methyl-1H-pyrazol-4-yl)pyridazin-4(1H)-one (Intermediate #132 40 mg, 0.14 mmol) in DMSO (300 μL) was added. The reaction continued to stir at r.t. for overnight. The reaction mixture was concentrated in vacuo, redissolved in DMSO (1 mL) and purified by reverse phase preparative HPLC. Fractions containing pure compound were collected, the solvent was removed in vacuo. The residue was dissolved in DCM and transferred to a Bohdan block containing MP-Carbonate. DIPEA (5 μL) was added and the vessel was shaken for 4 hours at room temperature, filtered and concentrated. The residues were then dissolved in MeCN and water, frozen, and lyophilized to give 2-(1H-imidazol-1-yl)ethyl(3-{[1-(1-methyl-1H-pyrazol-4-yl)-4-oxo-1,4-dihydropyridazin-3-yl]methyl}phenyl)carbamate.

LRMS (ESI) calc'd for C21H21N7O3 [M+H]⁺: 420. Found: 420.

The following examples were prepared from Intermediates #132-135 and 137 according to Scheme 12 following a similar procedure described for Example #510, which can be achieved by those of ordinary skill in the art of organic synthesis.

| | | | Exact |
|---|---|---|---|
| | | | Mass |
| Example | Structure | IUPAC Name | [M + H]+ |
| 511 | 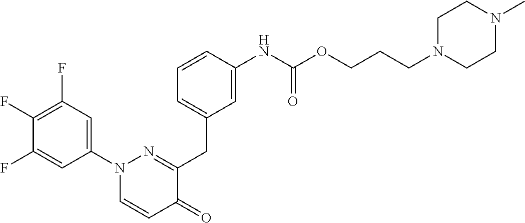 | 3-(4- methylpiperazin- 1-yl)propyl(3- {[4-oxo-1-(3,4,5- trifluorophenyl)- 1,4- dihydropyridazin- 3-yl]methyl} phenyl)carbamate | Calc'd 516, found 516 |
| 512 | 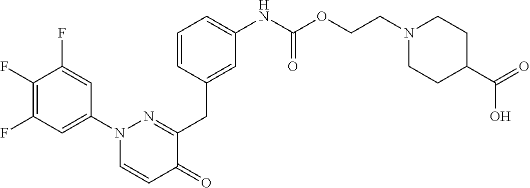 | 1-(2-{[(3-{[4- oxo-1-(3,4,5- trifluorophenyl)- 1,4- dihydropyridazin- 3-yl]methyl} phenyl)carbamoyl] oxy}ethyl) piperidine- 4-carboxylic acid | Calc'd 531, found 531 |
| 513 | 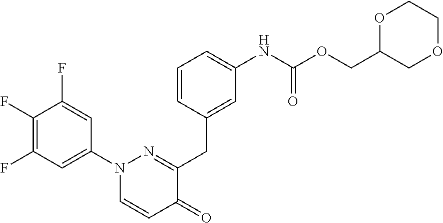 | rac-1,4-dioxan- 2-ylmethyl (3- {[4-oxo-1-(3,4,5- trifluorophenyl)- 1,4- dihydropyridazin- 3-yl]methyl} phenyl)carbamate | Calc'd 476, found 476 |
| 514 | 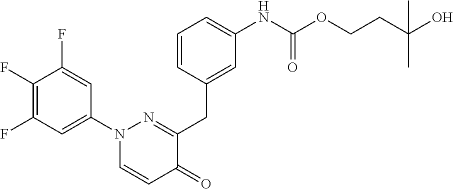 | 3-hydroxy-3- methylbutyl (3- {[4-oxo-1-(3,4,5- trifluorophenyl)- 1,4- dihydropyridazin- 3-yl]methyl} phenyl)carbamate | Calc'd 462, found 462 |
| 515 | 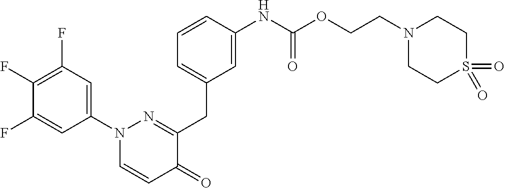 | 2-(1,1-dioxidothio- morpholin-4- yl)ethyl (3-{[4- oxo-1-(3,4,5- trifluorophenyl)- 1,4- dihydropyridazin- 3-yl]methyl} phenyl)carbamate | Calc'd 537, found 537 |
| 516 | 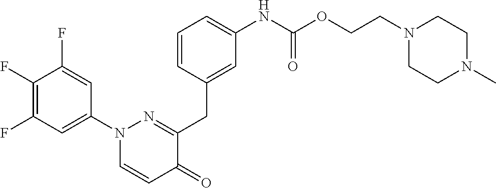 | 2-(4- methylpiperazin- 1-yl)ethyl (3- {[4-oxo-1-(3,4,5- trifluorophenyl)- 1,4- dihydropyridazin- 3-yl]methyl} phenyl)carbamate | Calc'd 502, found 502 |
| 517 | 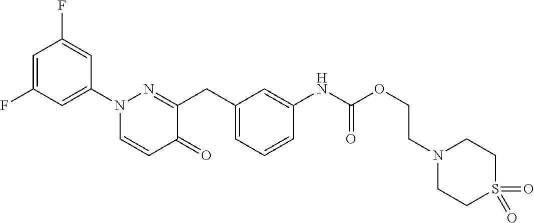 | 2-(1,1- dioxidothio- morpholin-4- yl)ethyl (3-{[1- (3,5- difluorophenyl)- 4-oxo-1,4- dihydropyridazin- 3-yl]methyl} phenyl)carbamate | Calc'd 519, found 519 |
| 518 | 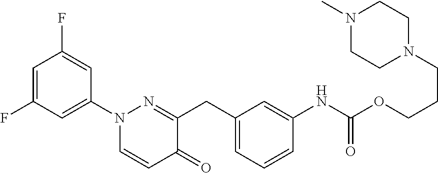 | 3-(4- methylpiperazin- 1-yl)ethyl (3- {[1-(3,5- difluorophenyl)- 4-oxo-1,4- dihydropyridazin- 3-yl]methyl} phenyl)carbamate | Calc'd 498, found 498 |
| 519 | 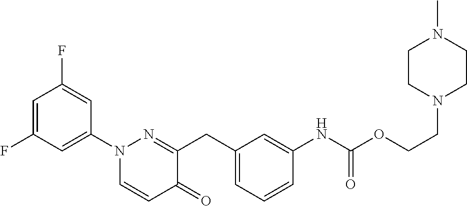 | 2-(4- methylpiperazin- 1-yl)ethyl (3- {[1-(3,5- difluorophenyl)- 4-oxo-1,4- dihydropyridazin- 3-yl]methyl} phenyl)carbamate | Calc'd 484, found 484 |
| 520 | 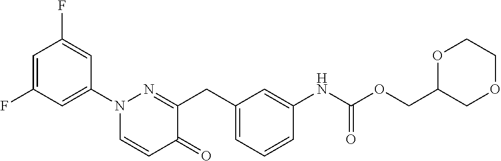 | rac-1,4-dioxan- 2-ylmethyl (3- {[1-(3,5- difluorophenyl)- 4-oxo-1,4- dihydropyridazin- 3-yl]methyl} phenyl)carbamate | Calc'd 458, found 458 |
| 521 | 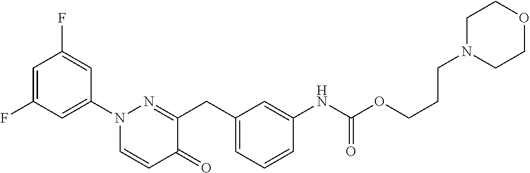 | 3-morpholin-4- ylpropyl (3- {[1-(3,5- difluorophenyl)- 4-oxo-1,4- dihydropyridazin- 3-yl]methyl} phenyl)carbamate | Calc'd 485, found 485 |
| 522 | 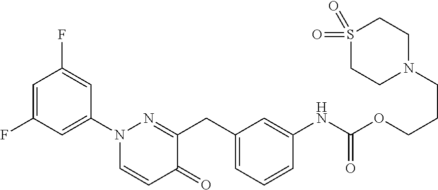 | 3-(1,1-dioxidothio- morpholin-4- yl)propyl (3- {[1-(3,5- difluorophenyl)- 4-oxo-1,4- dihydropyridazin- 3-yl]methyl} phenyl)carbamate | Calc'd 533, found 533 |
| 523 | 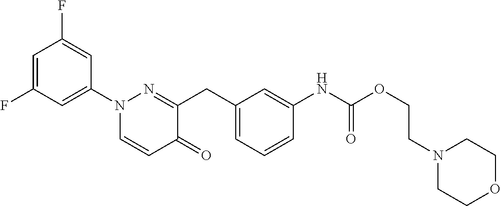 | 2-morpholin-4- ylethyl (3-{[1-(3,5- difluorophenyl)- 4-oxo-1,4- dihydropyridazin- 3-yl]methyl} phenyl)carbamate | Calc'd 471, found 471 |
| 524 | 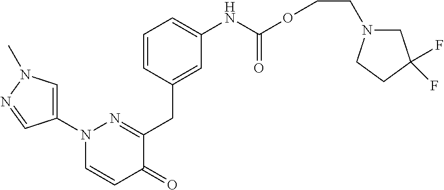 | 2-(3,3- difluoropyrrolidin- 1-yl)ethyl (3- {[1-(1-methyl- 1H-pyrazol-4- yl)-4-oxo-1,4- dihydropyridazin- 3-yl]methyl} phenyl)carbamate | Calc'd 459, found 459 |
| 525 | 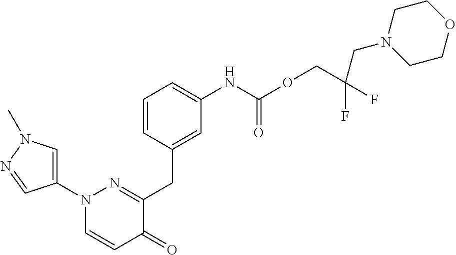 | 2,2-difluoro-3- morpholin-4- ylpropyl (3- {[1-(1-methyl- 1H-pyrazol-4- yl)-4-oxo-1,4- dihydropyridazin- 3-yl]methyl} phenyl)carbamate | Calc'd 489, found 489 |
| 526 | 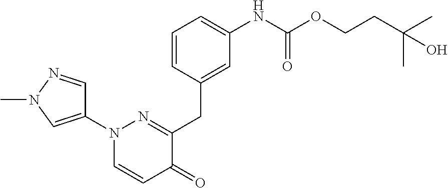 | 3-hydroxy-3- methylbutyl (3- {[1-(1-methyl- 1H-pyrazol-4- yl)-4-oxo-1,4- dihydropyridazin- 3-yl]methyl} phenyl)carbamate | Calc'd 412, found 412 |
| 527 | 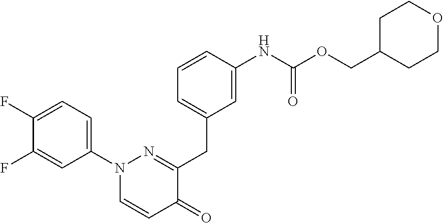 | tetrahydro-2H- pyran-4-ylmethyl (3-{[1-(3,4- difluorophenyl)- 4-oxo-1,4- dihydropyridazin- 3-yl]methyl} phenyl)carbamate | Calc'd 456, found 456 |
| 528 | 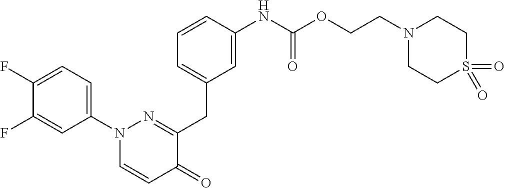 | 2-(1,1-dioxidothio- morpholin-4-yl) ethyl-(3-{[1-(3,4- difluorophenyl)- 4-oxo-1,4- dihydropyridazin- 3-yl]methyl} phenyl)carbamate | Calc'd 519, found 519 |
| 529 | 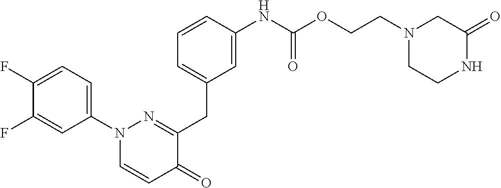 | 2-(3- oxopiperazin-1- yl)ethyl (3-{[1- (3,4- difluorophenyl)- 4-oxo-1,4- dihydropyridazin- 3-yl]methyl} phenyl)carbamate | Calc'd 484, found 484 |
| 530 | 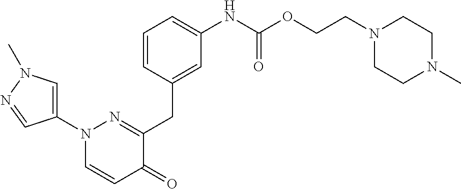 | 2-(4- methylpiperazin- 1-yl)ethyl (3- {[1-(1-methyl- 1H-pyrazol-4- yl)-4-oxo-1,4- dihydropyridazin- 3-yl]methyl} phenyl)carbamate | Calc'd 452, found 452 |
| 531 | 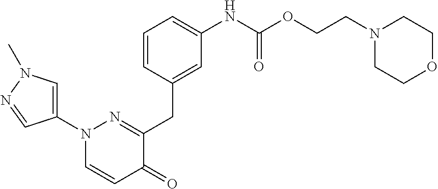 | 2-morpholin-4- yl)ethyl (3- {[1-(1-methyl- 1H-pyrazol-4- yl)-4-oxo-1,4- dihydropyridazin- 3-yl]methyl} phenyl)carbamate | Calc'd 439, found 439 |
| 532 | 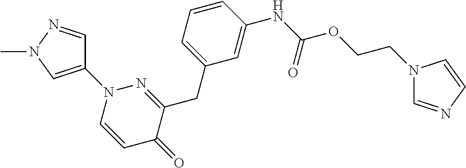 | 2-(1H-imidazol- 1-yl)ethyl (3- {[1-(1-methyl- 1H-pyrazol-4- yl)-4-oxo-1,4- dihydropyridazin- 3-yl]methyl} phenyl)carbamate | Calc'd 420, found 420 |
| 533 | 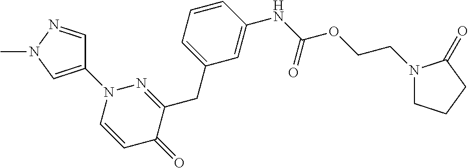 | 2-(2-oxopyrrolidin- 1-yl)ethyl (3- {[1-(1-methyl- 1H-pyrazol-4- yl)-4-oxo-1,4- dihydropyridazin- 3-yl]methyl} phenyl)carbamate | Calc'd 437, found 437 |
| 534 | 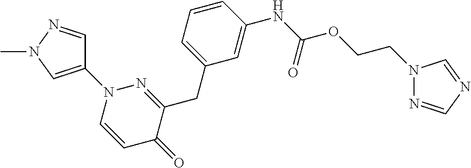 | 2-(1H-1,2,4- triazol-1-yl)ethyl (3-{[1-(1-methyl- 1H-pyrazol-4- yl)-4-oxo-1,4- dihydropyridazin- 3-yl]methyl} phenyl)carbamate | Calc'd 421, found 421 |
| 535 | 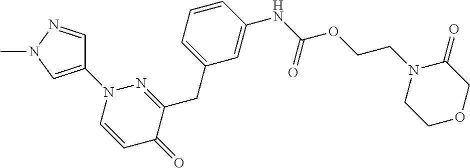 | 2-(3-oxomorpholin- 4-yl)ethyl (3- {[1-(1-methyl- 1H-pyrazol-4- yl)-4-oxo-1,4- dihydropyridazin- 3-yl]methyl} phenyl)carbamate | Calc'd 453, found 453 |
| 536 | 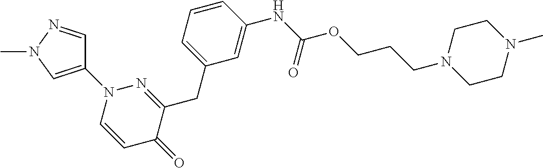 | 3-(4- methylpiperazin- 1-yl)propyl-(3- {[1-(1-methyl- 1H-pyrazol-4- yl)-4-oxo-1,4- dihydropyridazin- 3-yl]methyl} phenyl)carbamate | Calc'd 466, found 466 |
| 537 | 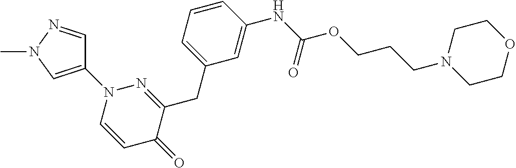 | 3-morpholin-4- ylpropyl (3-{[1- (1-methyl-1H- pyrazol-4-yl)- 4-oxo-1,4- dihydropyridazin- 3-yl]methyl} phenyl)carbamate | Calc'd 453, found 453 |
| 538 | 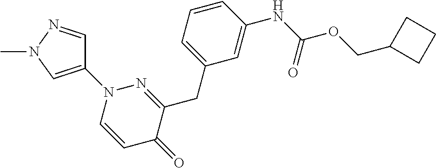 | cyclobutylmethyl (3-{[1-(1-methyl- 1H-pyrazol-4- yl)-4-oxo-1,4- dihydropyridazin- 3-yl]methyl} phenyl)carbamate | Calc'd 394, found 394 |
| 539 | 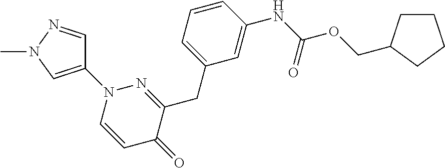 | cyclopentylmethyl (3-{[1-(1-methyl- 1H-pyrazol-4- yl)-4-oxo-1,4- dihydropyridazin- 3-yl]methyl} phenyl)carbamate | Calc'd 408, found 408 |
| 540 | 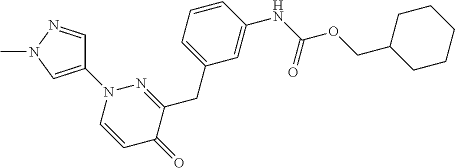 | cyclohexylmethyl (3-{[1-(1-methyl- 1H-pyrazol-4- yl)-4-oxo-1,4- dihydropyridazin- 3-yl]methyl} phenyl)carbamate | Calc'd 422, found 422 |
| 541 | 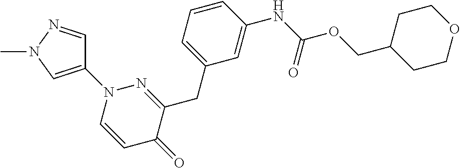 | tetrahydro-2H- pyran-4-ylmethyl (3-{[1-(1-methyl- 1H-pyrazol-4- yl)-4-oxo-1,4- dihydropyridazin- 3-yl]methyl} phenyl)carbamate | Calc'd 424, found 424 |
| 542 | 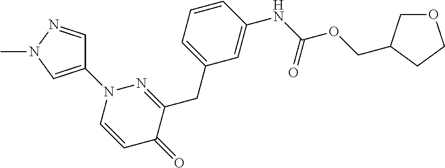 | rac- tetrahydrofuran- 3-ylmethyl (3- {[1-(1-methyl- 1H-pyrazol-4- yl)-4-oxo-1,4- dihydropyridazin- 3-yl]methyl} phenyl)carbamate | Calc'd 410, found 410 |
| 543 | 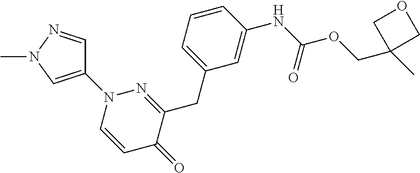 | (3-methyloxetan- 3-yl)methyl (3- {[1-(1-methyl- 1H-pyrazol-4- yl)-4-oxo-1,4- dihydropyridazin- 3-yl]methyl} phenyl)carbamate | Calc'd 410, found 410 |
| 544 | 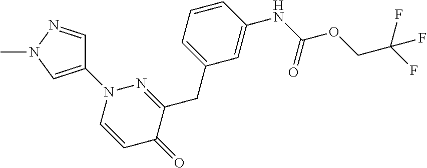 | 2,2,2-trifluoroethyl (3-{[1-(1-methyl- 1H-pyrazol-4-yl)- 4-oxo-1,4- dihydropyridazin- 3-yl]methyl} phenyl)carbamate | Calc'd 408, found 408 |
| 545 | 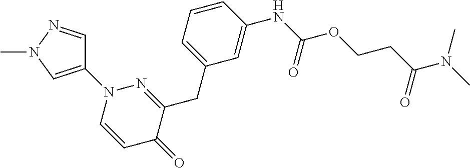 | 3-(dimethylamino)- 3-oxopropyl (3-{[1-(1- methyl-1H- pyrazol-4-yl)- 4-oxo-1,4- dihydropyridazin- 3-yl]methyl} phenyl)carbamate | Calc'd 425, found 425 |
| 546 | 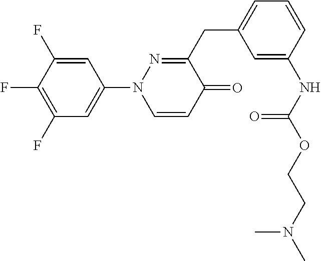 | 2-(dimethylamino) ethyl (3-{[4- oxo-1-(3,4,5- trifluorophenyl)- 1,4- dihydropyridazin- 3-yl]methyl} phenyl)carbamate | Calc'd 447, found 447 |
| 547 | 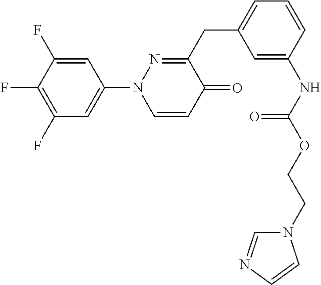 | 2-(1H-imidazol- 1-yl)ethyl (3- {[4-oxo-1-(3,4,5- trifluorophenyl)- 1,4- dihydropyridazin- 3-yl]methyl} phenyl)carbamate | Calc'd 470, found 470 |
| 548 | 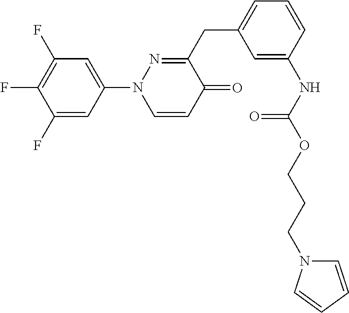 | 3-(1H-pyrrol-1- yl)propyl (3- {[4-oxo-1-(3,4,5- trifluorophenyl)- 1,4- dihydropyridazin- 3-yl]methyl} phenyl)carbamate | Calc'd 483, found 483 |
| 549 | 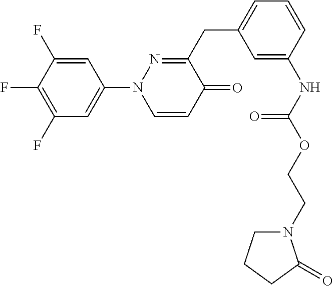 | 2-(2-oxopyrrolidin- 1-yl)ethyl (3- {[4-oxo-1-(3,4,5- trifluorophenyl)- 1,4- dihydropyridazin- 3-yl]methyl} phenyl)carbamate | Calc'd 487, found 487 |
| 550 | 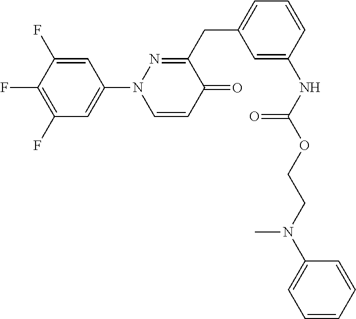 | 2-[methyl(phenyl) amino]ethyl (3- {[4-oxo-1-(3,4,5- trifluorophenyl)- 1,4- dihydropyridazin- 3-yl]methyl} phenyl)carbamate | Calc'd 509, found 509 |
| 551 | 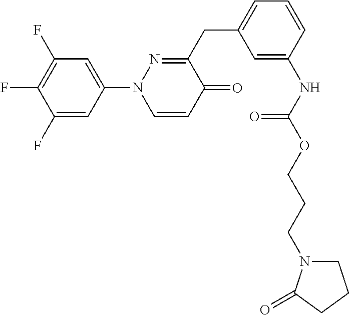 | 3-(2-oxopyrrolidin- 1-yl)propyl (3- {[4-oxo-1-(3,4,5- trifluorophenyl)- 1,4- dihydropyridazin- 3-yl]methyl} phenyl)carbamate | Calc'd 501, found 501 |
| 552 | 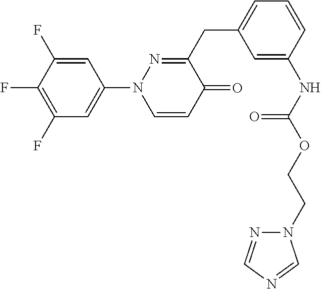 | 2-(1H-1,2,4-triazol- 1-yl)ethyl (3-{[4- oxo-1-(3,4,5- trifluorophenyl)- 1,4- dihydropyridazin- 3-yl]methyl} phenyl)carbamate | Calc'd 471, found 471 |
| 553 | 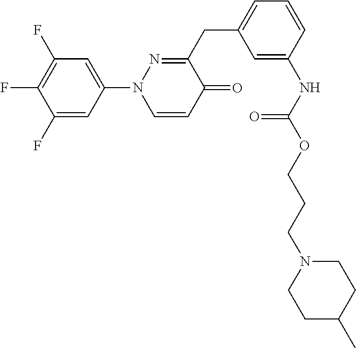 | 3-(4- methylpiperidin- 1-yl)propyl (3- {[4-oxo-1-(3,4,5- trifluorophenyl)- 1,4- dihydropyridazin- 3-yl]methyl} phenyl)carbamate | Calc'd 515, found 515 |
| 554 | 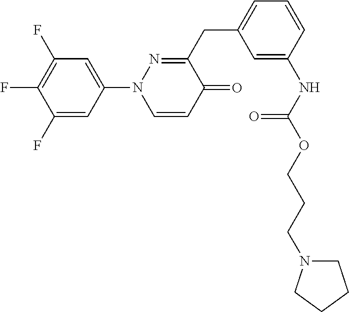 | 3-pyrrolidin-1- ylpropyl (3-{[4- oxo-1-(3,4,5- trifluorophenyl)- 1,4- dihydropyridazin- 3-yl]methyl} phenyl)carbamate | Calc'd 487, found 487 |
| 555 | 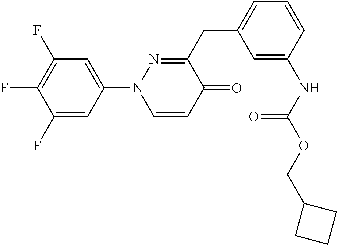 | cyclobutylmethyl (3-{[4-oxo-1- (3,4,5- trifluorophenyl)- 1,4- dihydropyridazin- 3-yl]methyl} phenyl)carbamate | Calc'd 444, found 444 |
| 556 | 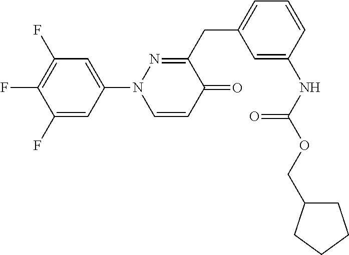 | cyclopentylmethyl (3-{[4-oxo-1- (3,4,5- trifluorophenyl)- 1,4- dihydropyridazin- 3-yl]methyl} phenyl)carbamate | Calc'd 458, found 458 |
| 557 | 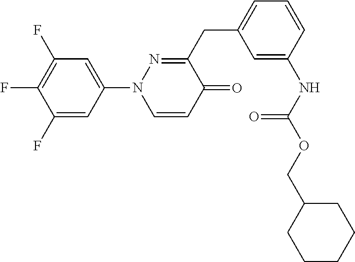 | cyclohexylmethyl (3-{[4-oxo-1- (3,4,5- trifluorophenyl)- 1,4- dihydropyridazin- 3-yl]methyl} phenyl)carbamate | Calc'd 472, found 472 |
| 558 | 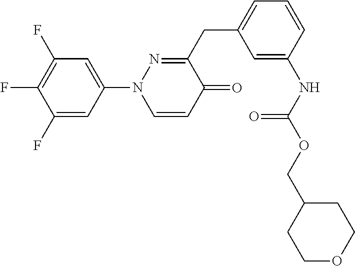 | tetrahydro-2H- pyran-4-ylmethyl (3-{[4-oxo-1- (3,4,5- trifluorophenyl)- 1,4- dihydropyridazin- 3-yl]methyl} phenyl)carbamate | Calc'd 474, found 474 |
| 559 | 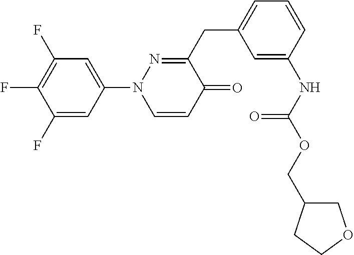 | rac- tetrahydrofuran- 3-ylmethyl (3- {[4-oxo-1-(3,4,5- trifluorophenyl)- 1,4- dihydropyridazin- 3-yl]methyl} phenyl)carbamate | Calc'd 460, found 460 |
| 560 | 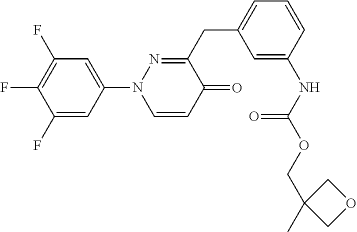 | (3-methyloxetan- 3-yl)methyl (3- {[4-oxo-1-(3,4,5- trifluorophenyl)- 1,4- dihydropyridazin- 3-yl]methyl} phenyl)carbamate | Calc'd 460, found 460 |
| 561 | 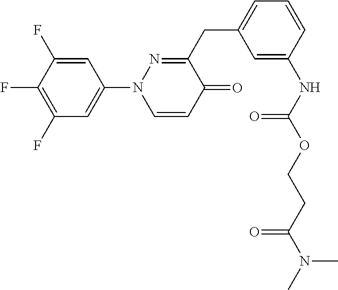 | 3-(dimethylamino)- 3-oxopropyl (3- {[4-oxo-1-(3,4,5- trifluorophenyl)- 1,4- dihydropyridazin- 3-yl]methyl} phenyl)carbamate | Calc'd 475, found 475 |
| 562 | 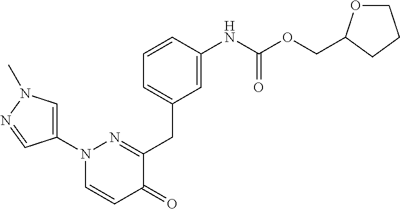 | rac- tetrahydrofuran- 2-ylmethyl (3- {[1-(1-methyl- 1H-pyrazol-4- yl)-4-oxo-1,4- dihydropyridazin- 3-yl]methyl} phenyl)carbamate | Calc'd 410, found 410 |
| 563 | 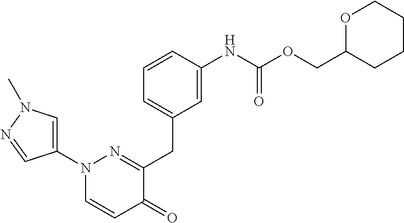 | rac-tetrahydro- 2H-pyran-2- ylmethyl (3- {[1-(1-methyl- 1H-pyrazol-4- yl)-4-oxo-1,4- dihydropyridazin- 3-yl]methyl} phenyl)carbamate | Calc'd 424, found 424 |
| 564 | 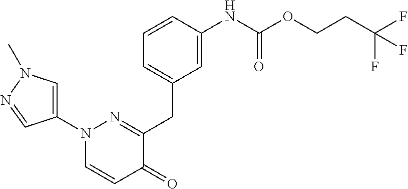 | 3,3,3- trifluoropropyl (3-{[1-(1-methyl- 1H-pyrazol-4- yl)-4-oxo-1,4- dihydropyridazin- 3-yl]methyl} phenyl)carbamate | Calc'd 422, found 422 |
| 565 | 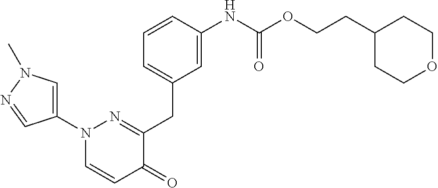 | 2-(tetrahydro-2H- pyran-4-yl)ethyl (3-{[1-(1-methyl- 1H-pyrazol-4- yl)-4-oxo-1,4- dihydropyridazin- 3-yl]methyl} phenyl)carbamate | Calc'd 438, found 438 |
| 566 | 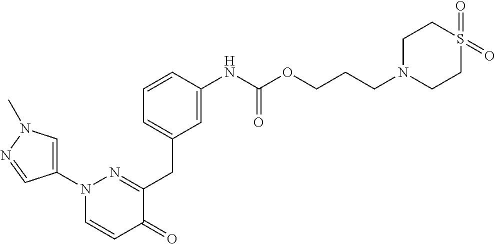 | 3-(1,1-dioxidothio- morpholin-4- yl)propyl (3- {[1-(1-methyl- 1H-pyrazol-4- yl)-4-oxo-1,4- dihydropyridazin- 3-yl]methyl} phenyl)carbamate | Calc'd 501, found 501 |
| 567 | 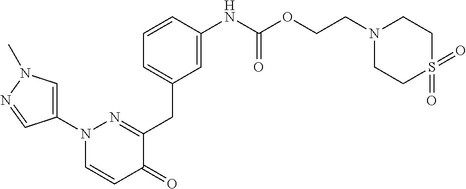 | 2-(1,1-dioxidothio- morpholin-4- yl)ethyl (3- {[1-(1-methyl- 1H-pyrazol-4- yl)-4-oxo-1,4- dihydropyridazin- 3-yl]methyl} phenyl)carbamate | Calc'd 487, found 487 |
| 568 | 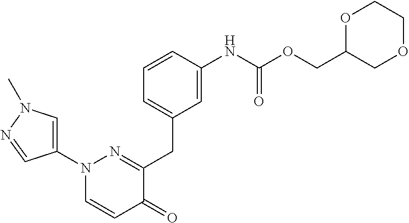 | rac-1,4-dioxan- 2-ylmethyl (3- {[1-(1-methyl- 1H-pyrazol-4- yl)-4-oxo-1,4- dihydropyridazin- 3-yl]methyl} phenyl)carbamate | Calc'd 426, found 426 |
| 569 | 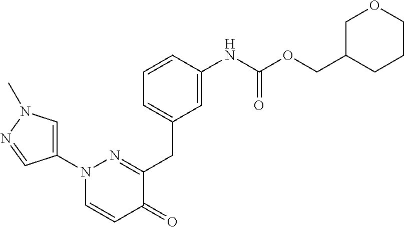 | rac-tetrahydro- 2H-pyran-3- ylmethyl (3- {[1-(1-methyl- 1H-pyrazol-4- yl)-4-oxo-1,4- dihydropyridazin- 3-yl]methyl} phenyl)carbamate | Calc'd 424, found 424 |
| 570 | 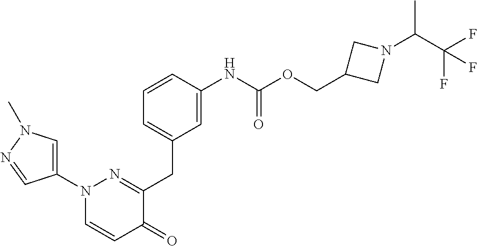 | rac-[1-(2,2,2- trifluoro-1- methylethyl) azetidin-3- yl]methyl (3- {[1-(1-methyl- 1H-pyrazol-4- yl)-4-oxo-1,4- dihydropyridazin- 3-yl]methyl} phenyl)carbamate | Calc'd 491, found 491 |
| 571 | 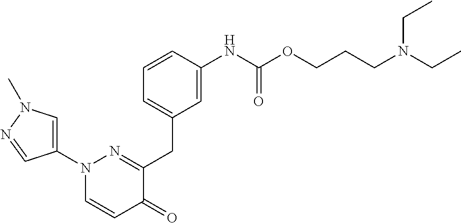 | 3-(diethylamino) propyl (3-{[1- (1-methyl- 1H-pyrazol-4- yl)-4-oxo-1,4- dihydropyridazin- 3-yl]methyl} phenyl)carbamate | Calc'd 439, found 439 |
| 572 | 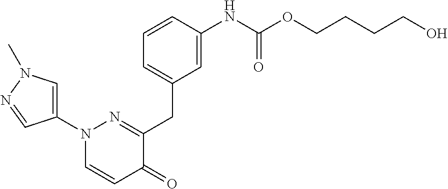 | 4-hydroxybutyl (3-{[1-(1-methyl- 1H-pyrazol-4- yl)-4-oxo-1,4- dihydropyridazin- 3-yl]methyl} phenyl)carbamate | Calc'd 398, found 398 |
| 573 | 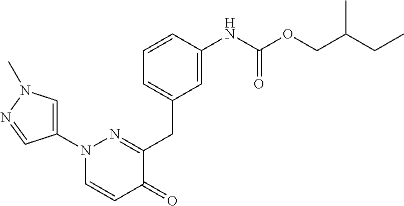 | rac-2-methylbutyl (3-{[1-(1-methyl- 1H-pyrazol-4- yl)-4-oxo-1,4- dihydropyridazin- 3-yl]methyl} phenyl)carbamate | Calc'd 396, found 396 |
| 574 | 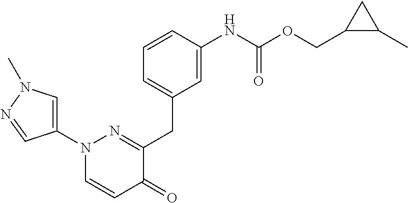 Mixture of isomers | (2- methylcyclopropyl) methyl (3-{[1-(1- methyl-1H-pyrazol- 4-yl)-4-oxo-1,4- dihydropyridazin- 3-yl]methyl} phenyl)carbamate | Calc'd 394, found 394 |
| 575 | 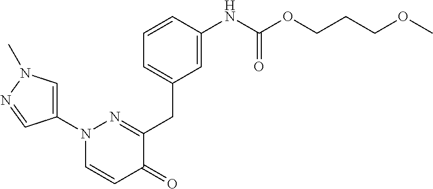 | 3-methoxypropyl (3-{[1-(1-methyl- 1H-pyrazol-4- yl)-4-oxo-1,4- dihydropyridazin- 3-yl]methyl} phenyl)carbamate | Calc'd 398, found 398 |
| 576 | 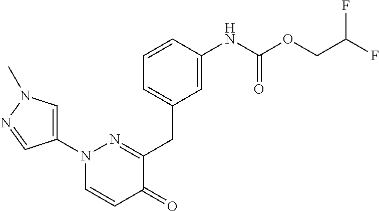 | 2,2-difluoroethyl (3-{[1-(1-methyl- 1H-pyrazol-4- yl)-4-oxo-1,4- dihydropyridazin- 3-yl]methyl} phenyl)carbamate | Calc'd 390, found 390 |
| 577 | 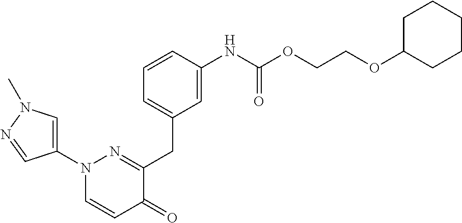 | 2-(cyclohexyloxy) ethyl (3-{[1-(1- methyl-1H-pyrazol- 4-yl)-4-oxo-1,4- dihydropyridazin- 3-yl]methyl} phenyl)carbamate | Calc'd 452, found 452 |
| 578 | 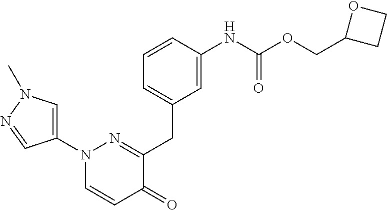 | rac-oxetan-2- ylmethyl (3-{[1-(1- methyl-1H-pyrazol- 4-yl)-4-oxo-1,4- dihydropyridazin- 3-yl]methyl} phenyl)carbamate | Calc'd 396, found 396 |
| 579 | 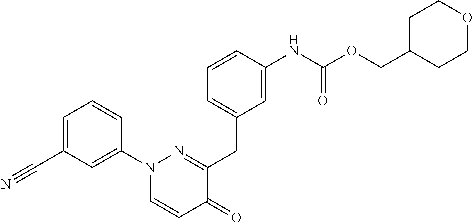 | tetrahydro-2H- pyran-4- ylmethyl (3-{[1- (3-cyanophenyl)- 4-oxo-1,4- dihydropyridazin- 3-yl]methyl} phenyl)carbamate | Calc'd 445 found, 445 |
| 580 | 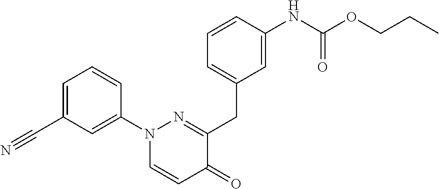 | propyl (3-{[1-(3- cyanophenyl)-4- oxo-1,4- dihydropyridazin- 3-yl]methyl} phenyl)carbamate | Calc'd 389, found 389 |
| 581 | 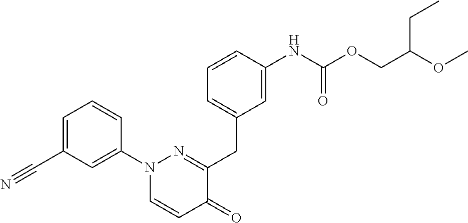 | rac-2-methoxybutyl (3-{[1-(3- cyanophenyl)-4- oxo-1,4- dihydropyridazin- 3-yl]methyl} phenyl)carbamate | Calc'd 433, found 433 |
| 582 | 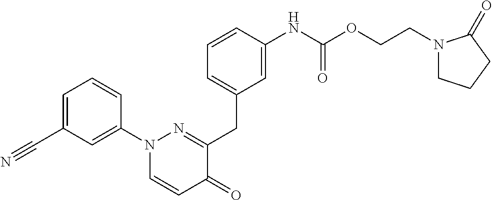 | 2-(2-oxopyrrolidin- 1-yl)ethyl (3-{[1- (3-cyanophenyl)- 4-oxo-1,4- dihydropyridazin- 3-yl]methyl} phenyl)carbamate | Calc'd 458, found 458 |
| 583 | 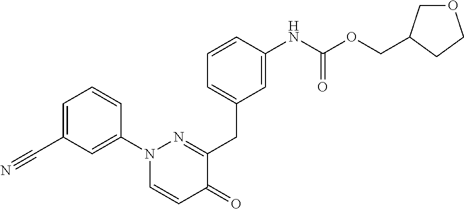 | rac- tetrahydrofuran- 3-ylmethyl (3-{[1- (3-cyanophenyl)- 4-oxo-1,4- dihydropyridazin- 3-yl]methyl} phenyl)carbamate | Calc'd 431, found 431 |
| 584 | 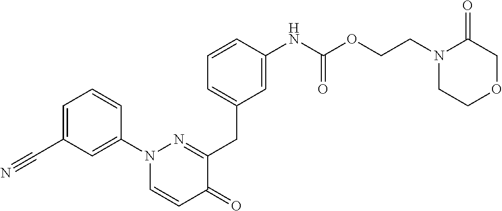 | 2-(3-oxomorpholin- 4-yl)ethyl (3-{[1- (3-cyanophenyl)- 4-oxo-1,4- dihydropyridazin- 3-yl]methyl} phenyl)carbamate | Calc'd 474, found 474 |
| 585 | 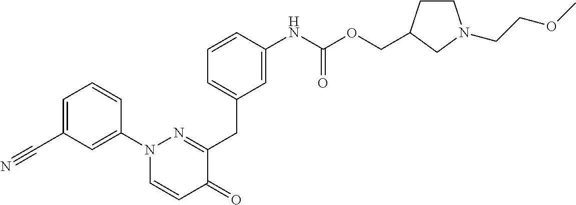 | rac-[1-(2- methoxyethyl) pyrrolidin-3- yl]methyl (3-{[1- (3-cyanophenyl)- 4-oxo-1,4- dihydropyridazin- 3-yl]methyl} phenyl)carbamate | Calc'd 488, found 488 |
| 586 | 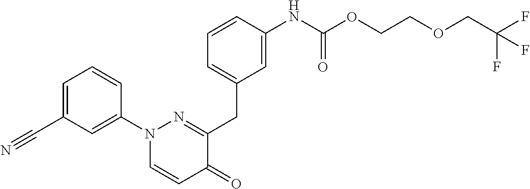 | 2-(2,2,2- trifluoroethoxy) ethyl (3-{[1- (3-cyanophenyl)- 4-oxo-1,4- dihydropyridazin- 3-yl]methyl} phenyl)carbamate | Calc'd 473, found 473 |
| 587 | 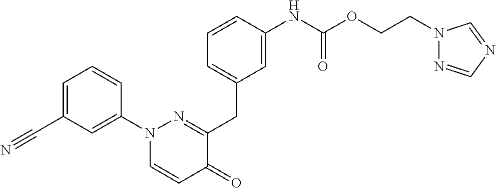 | 2-(1H-1,2,4- triazol-1-yl)ethyl (3-{[1- (3-cyanophenyl)- 4-oxo-1,4- dihydropyridazin- 3-yl]methyl} phenyl)carbamate | Calc'd 442, found 442 |
| 588 | 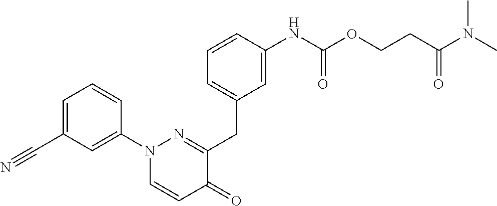 | 3-(dimethylamino)- 3-oxopropyl (3-{[1- (3-cyanophenyl)- 4-oxo-1,4- dihydropyridazin- 3-yl]methyl} phenyl)carbamate | Calc'd 446, found 446 |
| 589 | 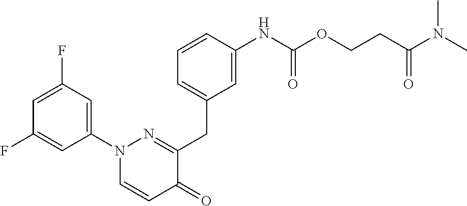 | 3-(dimethylamino)- 3-oxopropyl (3-{[1-(3,5- difluorophenyl)- 4-oxo-1,4- dihydropyridazin- 3-yl]methyl} phenyl)carbamate | Calc'd 457, found 457 |
| 590 | 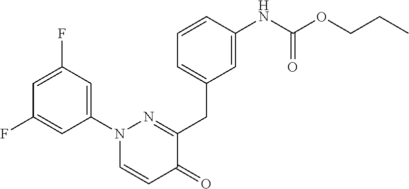 | propyl (3-{[1-(3,5- difluorophenyl)- 4-oxo-1,4- dihydropyridazin- 3-yl]methyl} phenyl)carbamate | Calc'd 400, found 400 |
| 591 | 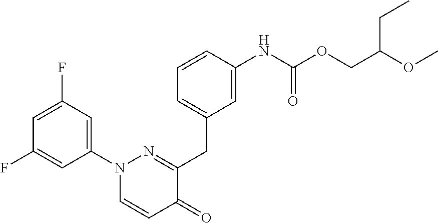 | rac-2-methoxybutyl (3-{[1-(3,5- difluorophenyl)- 4-oxo-1,4- dihydropyridazin- 3-yl]methyl} phenyl)carbamate | Calc'd 444, found 444 |
| 592 | 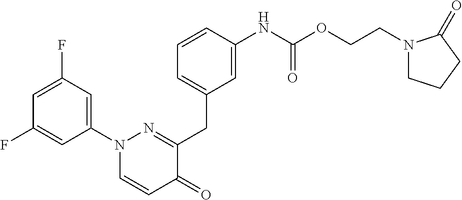 | 2-(2-oxopyrrolidin- 1-yl)ethyl (3- {[1-(3,5- difluorophenyl)- 4-oxo-1,4- dihydropyridazin- 3-yl]methyl} phenyl)carbamate | Calc'd 469, found 469 |
| 593 | 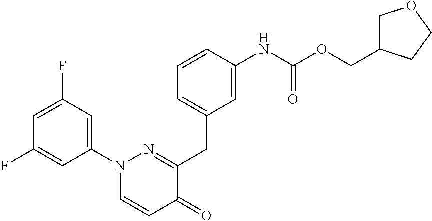 | rac- tetrahydrofuran- 3-ylmethyl-(3- {[1-(3,5- difluorophenyl)- 4-oxo-1,4- dihydropyridazin- 3-yl]methyl} phenyl)carbamate | Calc'd 442, found 442 |
| 594 | 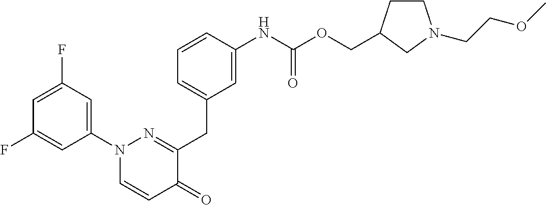 | rac-[1-(2- methoxyethyl) pyrrolidin-3- yl]methyl-(3- {[1-(3,5- difluorophenyl)- 4-oxo-1,4- dihydropyridazin- 3-yl]methyl} phenyl)carbamate | Calc'd 499, found 499 |
| 595 | 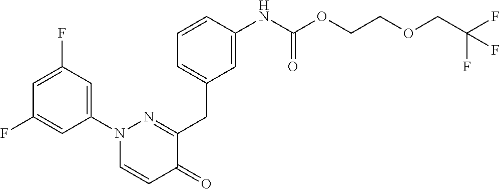 | 2-(2,2,2- trifluoroethoxy) ethyl (3-{[1-(3,5- difluorophenyl)- 4-oxo-1,4- dihydropyridazin- 3-yl]methyl} phenyl)carbamate | Calc'd 484, found 484 |
| 596 | 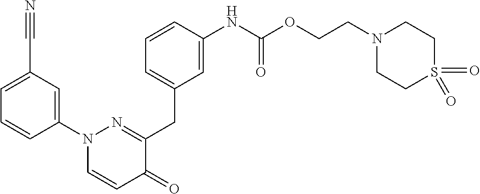 | 2-(1,1-dioxidothio- morpholin-4-yl) ethyl (3-{[1-(3- cyanophenyl)- 4-oxo-1,4- dihydropyridazin- 3-yl]methyl} phenyl)carbamate | Calc'd 508, found 508 |
| 597 | 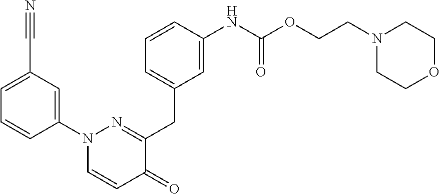 | 2-morpholin-4- ylethyl (3-{[1-(3- cyanophenyl)-4- oxo-1,4- dihydropyridazin- 3-yl]methyl} phenyl)carbamate | Calc'd 460, found 460 |
| 598 | 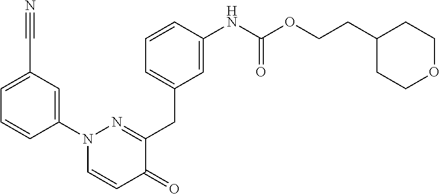 | 2-(tetrahydro- 2H-pyran-4- yl)ethyl (3-{[1-(3- cyanophenyl)-4- oxo-1,4- dihydropyridazin- 3-yl]methyl} phenyl)carbamate | Calc'd 459, found 459 |
| 599 | 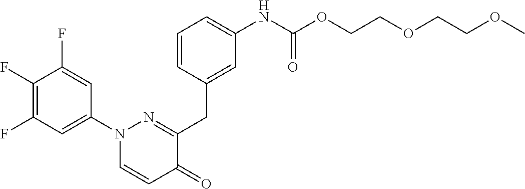 | 2-(2- methoxyethoxy) ethyl (3-{[4- oxo-1-(3,4,5- trifluorophenyl)- 1,4- dihydropyridazin- 3-yl]methyl} phenyl)carbamate | Calc'd 478, found 478 |
| 600 | 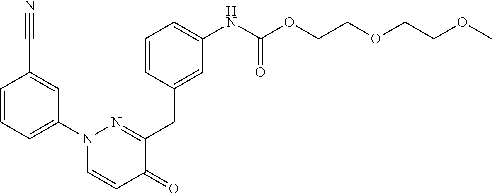 | 2-(2- methoxyethoxy) ethyl (3-{[1-(3- cyanophenyl)-4- oxo-1,4- dihydropyridazin- 3-yl]methyl} phenyl)carbamate | Calc'd 449, found 449 |
| 601 | 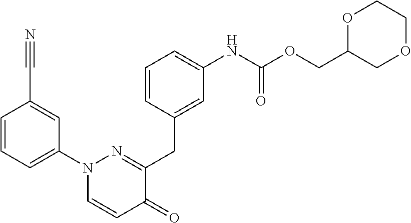 | rac-1,4-dioxan-2- ylmethyl (3-{[1- (3-cyanophenyl)- 4-oxo-1,4- dihydropyridazin- 3-yl]methyl} phenyl)carbamate | Calc'd 447, found 447 |
| 602 | 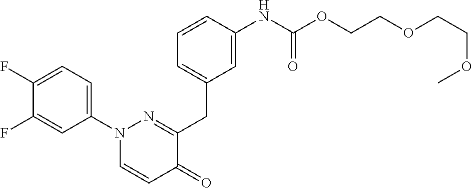 | 2-(2- methoxyethoxy) ethyl (3-{[1-(3,4- difluorophenyl)- 4-oxo-1,4- dihydropyridazin- 3-yl]methyl} phenyl)carbamate | Calc'd 460, found 460 |
| 603 | 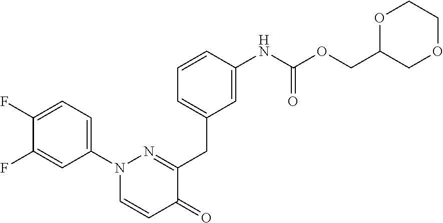 | rac-1,4-dioxan- 2-ylmethyl (3- {[1-(3,4- difluorophenyl)- 4-oxo-1,4- dihydropyridazin- 3-yl]methyl} phenyl)carbamate | Calc'd 458, found 458 |
| 604 | 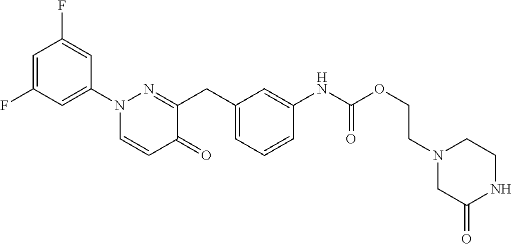 | 2-(3-oxopiperazin- 1-yl)ethyl (3-{[1- (3,5- difluorophenyl)- 4-oxo-1,4- dihydropyridazin- 3-yl]methyl} phenyl)carbamate | Calc'd 484, found 484 |
| 605 | 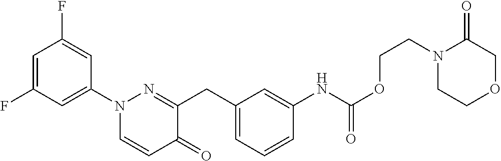 | 2-(3-oxomorpholin- 4-yl)ethyl (3- {[1-(3,5- difluorophenyl)- 4-oxo-1,4- dihydropyridazin- 3-yl]methyl} phenyl)carbamate | Calc'd 485, found 485 |
| 606 | 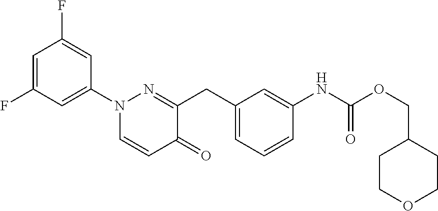 | tetrahydro-2H- pyran-4-ylmethyl (3-{[1-(3,5- difluorophenyl)- 4-oxo-1,4- dihydropyridazin- 3-yl]methyl} phenyl)carbamate | Calc'd 456, found 456 |
| 607 | 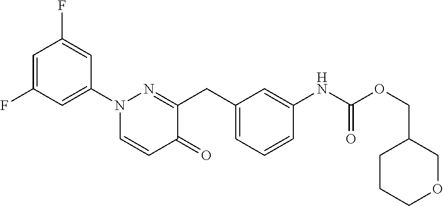 | rac-tetrahydro- 2H-pyran-3- ylmethyl (3-{[1- (3,5- difluorophenyl)- 4-oxo-1,4- dihydropyridazin- 3-yl]methyl} phenyl)carbamate | Calc'd 456, found 456 |
| 608 | 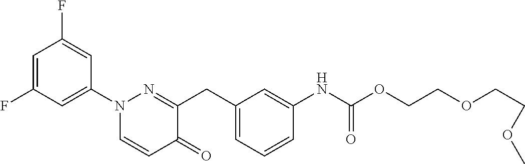 | 2-(2- methoxyethoxy) ethyl (3-{[1-(3,5- difluorophenyl)- 4-oxo-1,4- dihydropyridazin- 3-yl]methyl} phenyl)carbamate | Calc'd 460, found 460 |
| 609 | 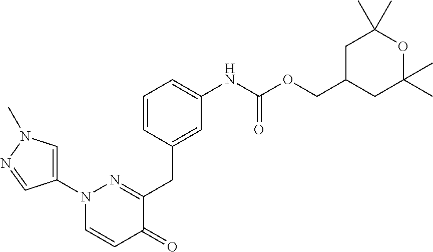 | (2,2,6,6- tetramethyltetra- hydro-2H-pyran- 4-yl)methyl (3- {[1-(1-methyl- 1H-pyrazol-4-yl)- 4-oxo-1,4- dihydropyridazin- 3-yl]methyl} phenyl)carbamate | Calc'd 480, found 480 |
| 610 | 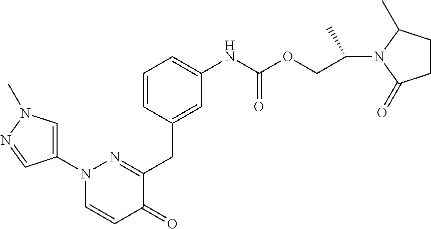 Single enantiomer | (2S)-2-[(2R or S)-2-methyl-5- oxopyrrolidin- 1-yl]propyl (3- {[1-(1-methyl- 1H-pyrazol-4-yl)- 4-oxo-1,4- dihydropyridazin- 3-yl]methyl} phenyl)carbamate | Calc'd 465, found 465 |
| 611 | 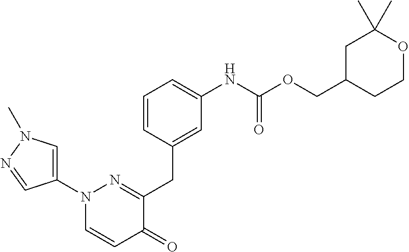 | rac-(2,2-dimethyl- tetrahydro-2H- pyran-4-yl)methyl (3-{[1-(1-methyl- 1H-pyrazol-4-yl)- 4-oxo-1,4- dihydropyridazin- 3-yl]methyl} phenyl)carbamate | Calc'd 452, found 452 |
| 612 | 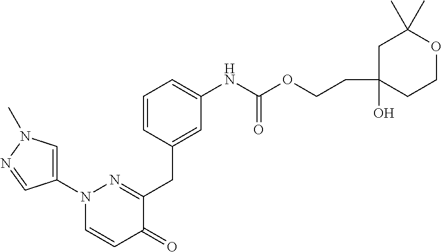 | rac-2-(4-hydroxy- 2,2-dimethyl- tetrahydro-2H- pyran-4-yl)ethyl (3-{[1-(1-methyl- 1H-pyrazol-4-yl)- 4-oxo-1,4- dihydropyridazin- 3-yl]methyl} phenyl)carbamate | Calc'd 482, found 482 |
| 613 | 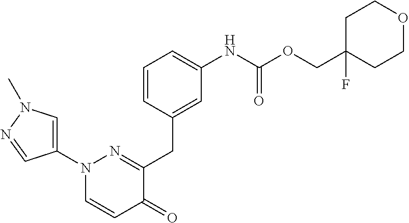 | (4- fluorotetrahydro- 2H-pyran-4- yl)methyl (3-{[1- (1-methyl- 1H-pyrazol-4-yl)- 4-oxo-1,4- dihydropyridazin- 3-yl]methyl} phenyl)carbamate | Calc'd 442, found 442 |

The following intermediates were prepared according to Scheme 12 following a similar procedure described for Example #510, which can be achieved by those of ordinary skill in the art of organic synthesis.

| | | | Exact |
|---|---|---|---|
| | | | Mass |
| Intermediate | Structure | IUPAC Name | [M + H]+ |
| 158 | 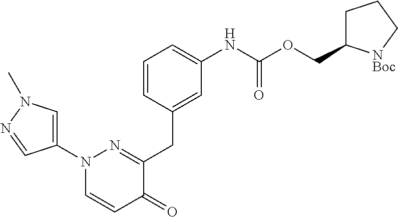 | tert-butyl (2R)- 2-({[(3-{[1-(1- methyl-1H- pyrazol-4-yl)-4- oxo-1,4- dihydropyridazin- 3-yl]methyl} phenyl)carbamoyl] oxy}methyl) pyrrolidine-1- carboxylate | Calc′d 509, found 509 |
| 159 | 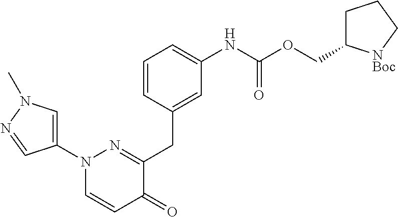 | tert-butyl (2S)- 2-({[(3-{[1-(1- methyl-1H- pyrazol-4-yl)-4- oxo-1,4- dihydropyridazin- 3-yl]methyl}- phenyl)carbamoyl] oxy}methyl) pyrrolidine-1- carboxylate | Calc′d 509, found 509 |
| 160 | 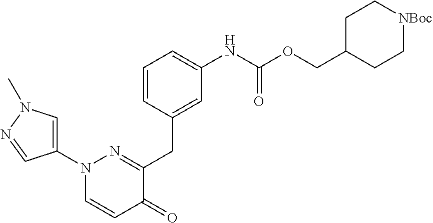 | tert-butyl 4- ({[(3-{[1-(1- methyl-1H- pyrazol-4-yl)-4- oxo-1,4- dihydropyridazin- 3-yl]methyl} phenyl)carbamoyl] oxy}methyl) piperidine-1- carboxylate | Calc′d 523, found 523 |
| 161 | 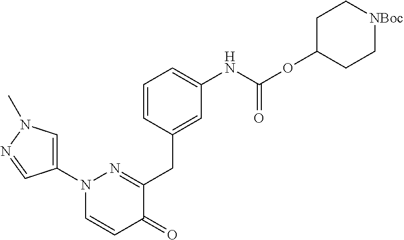 | tert-butyl 4- {[(3-{[1-(1- methyl-1H- pyrazol-4-yl)-4- oxo-1,4- dihydropyridazin- 3-yl]methyl} phenyl)carbamoyl] oxy}piperidine- 1-carboxylate | Calc′d 509, found 509 |
| 162 | 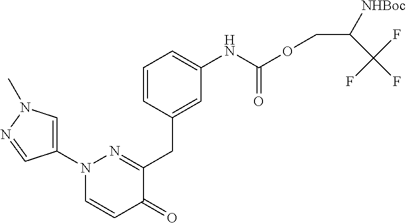 | rac-2-[(tert- butoxycarbonyl) amino]-3,3,3- trifluoropropyl (3-{[1-(1- methyl-1H- pyrazol-4-yl)-4- oxo-1,4- dihydropyridazin- 3-yl]methyl} phenyl)carbamate | Calc′d 537, found 537 |
| 163 | 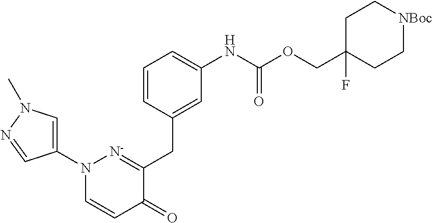 | tert-butyl 4- fluoro-4-({[(3- {[1-(1-methyl- 1H-pyrazol-4- yl)-4-oxo-1,4- dihydropyridazin- 3-yl]methyl} phenyl)caxbamoyl] oxy}methyl) piperidine-1- carboxylate | Calc′d 541, found 541 |
| 164 | 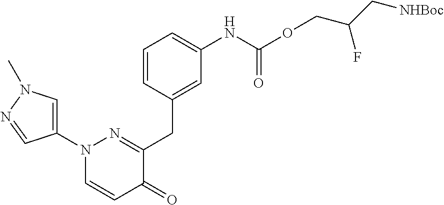 | rac-3-[(tert- butoxycarbonyl) amino]-2- fluoropropyl (3- {[1-(1-methyl- 1H-pyrazol-4- yl)-4-oxo-1,4- dihydropyridazin- 3-yl]methyl} phenyl)carbamate (non-preferred name) | Calc′d 501, found 501 |
| 165 | 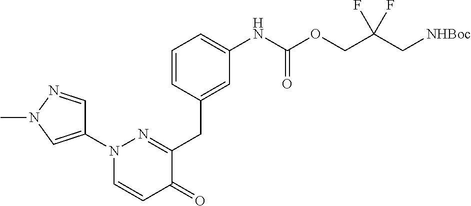 | 3-[(tert- butoxycarbonyl) amino]-2,2- difluoropropyl (3-{[1-(1- methyl-1H- pyrazol-4-yl)-4- oxo-1,4- dihydropyridazin- 3-yl]methyl} phenyl)carbamate (non-preferred name) | Calc′d 519, found 519 |
| 166 | 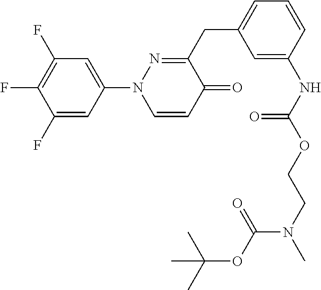 | 2-[(tert- butoxycarbonyl) (methyl)amino] ethyl(3-{[4- oxo-1-(3,4,5- trifluorophenyl)- 1,4-dihydropyridazin- 3-yl]methyl} phenyl)carbamate | nd |
| 167 | 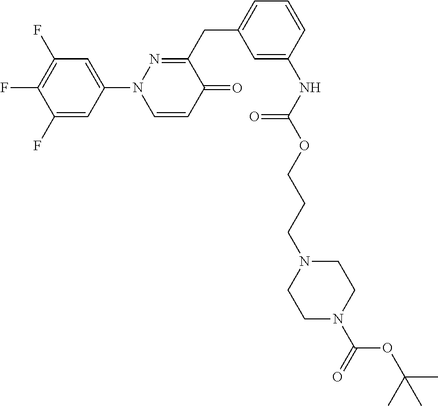 | tert-butyl 4-[3- ({[(3-{[4-oxo-1- (3,4,5-trifluorophenyl)- 1,4-dihydropyridazin- 3-yl]methyl} phenyl)amino] carbonyl}oxy)propyl] piperazine-1- carboxylate | nd |
| 168 | 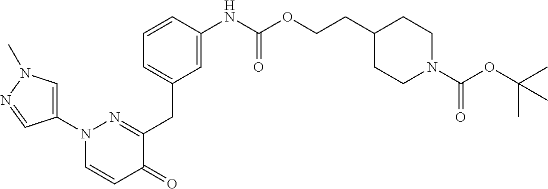 | tert-butyl 4-[2- ({[(3-{[1-(1- methyl-1H- pyrazol-4-yl)-4- oxo-1,4- dihydropyridazin- 3-yl]methyl} phenyl)amino] carbonyl}oxy)ethyl] piperidine-1- carboxylate | nd |
| 169 | 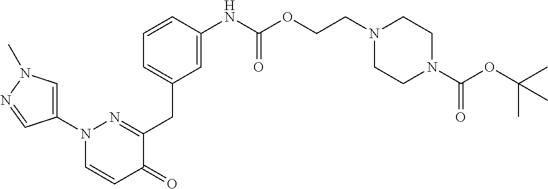 | tert-butyl 4-[2- ({[(3-{[1-(1- methyl-1H- pyrazol-4-yl)-4- oxo-1,4- dihydropyridazin- 3-yl]methyl} phenyl)amino] carbonyl}oxy)ethyl] piperazine-1- carboxylate | nd |
| 170 | 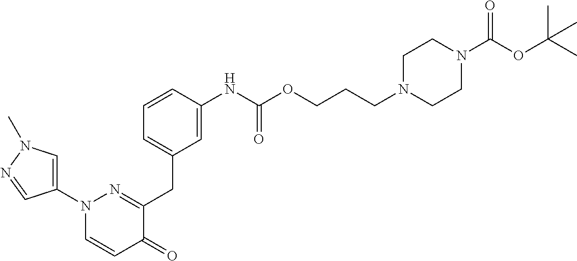 | tert-butyl 4-[3- ({[(3-{[1-(1- methyl-1H- pyrazol-4-yl)-4- oxo-1,4- dihydropyridazin- 3-yl]methyl} phenyl)amino] carbonyl}oxy)propyl] piperazine-1- carboxylate | nd |
| 171 | 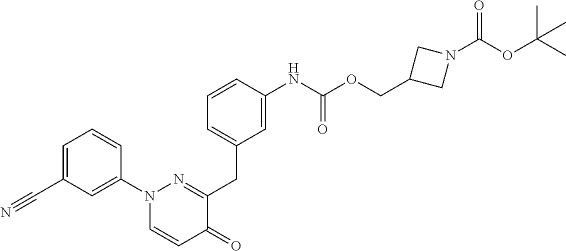 | tert-butyl 3- [({[(3-{[1-(3- cyanophenyl)-4- oxo-1,4- dihydropyridazin- 3-yl]methyl} phenyl)amino] carbonyl}oxy)methyl] azetidine-1- carboxylate | nd |
| 172 | 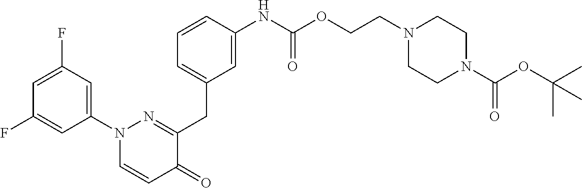 | tert-butyl 4-[2- ({[(3-{[1-(3,5- difluorophenyl)- 4-oxo-1,4- dihydropyridazin- 3-yl]methyl} phenyl)amino] carbonyl}oxy)ethyl] piperazine-1- carboxylate | nd |
| 173 | 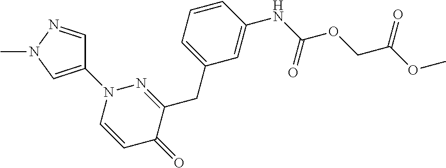 | methyl ({[(3- {[1-(1-methyl- 1H-pyrazol-4- yl)-4-oxo-1,4- dihydropyridazin- 3-yl]methyl} phenyl)amino] carbonyl}oxy)acetate | Calc′d 398, found 398 |
| 174 | 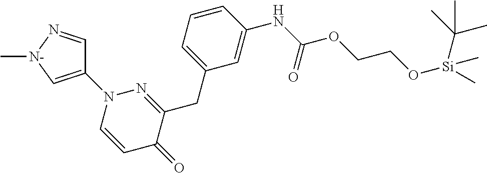 | 2-{[tert- butyl(dimethyl) silyl]oxy}ethyl (3-{[1-(1- methyl-1H- pyrazol-4-yl)-4- oxo-1,4- dihydropyridazin- 3-yl]methyl} phenyl)carbamate | Calc′d 484, found 484 |

Procedures for the preparation of the alcohols (Intermediates #193-203) used in the synthesis of Examples #513, 520, 524, 525, 568, 570, 601, 603, 609, 610 and 6-13 and Intermediates #162-165 are shown below.

### Scheme 12

### Example #614

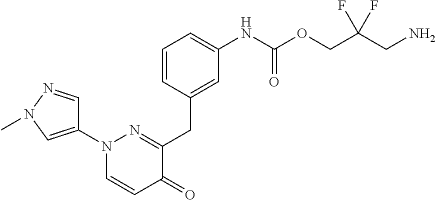

### 3-Amino-2,2-difluoropropyl (3-{[1-(1-methyl-1H-pyrazol-4-yl)-4-oxo-1,4-dihydropyridazin-3-yl]methyl}phenyl)carbamate

### Step 1. 3-Amino-2,2-difluoropropyl(3-{[1-(1-methyl-1H-pyrazol-4-yl)-4-oxo-1,4-dihydropyridazin-3-yl]methyl}phenyl)carbamate

3-[(tert-Butoxycarbonyl)amino]-2,2-difluoropropyl (3-{[1-(1-methyl-1H-pyrazol-4-yl)-4-oxo-1,4-dihydropyridazin-3-yl]methyl}phenyl)carbamate (Intermediate #165, 56 mg, 0.11 mmol) was added to a vial, followed by 1,4-dioxane (1 mL). 4 M HCl in 1,4-dioxane (0.5 mL) was added. The reaction mixture was stirred at room temperature for 3 hrs. The reaction mixture was diluted with EtOAc and filtered. The resulting filter cake was dissolved in MeOH, and solution was concentrated to a crude solid, dissolved in MeOH and DMSO, and purified by reverse phase preparative HPLC (0-50% MeCN—H₂O, 0.05% TFA). Fractions containing pure compound were collected and the free base was liberated using PL-HCO₃ cartridges (Stratospheres™, 0.9 mmol). The filtrate was frozen and freeze dried to give 3-amino-2,2-difluoropropyl (3-{[1-(1-methyl-1-pyrazol-4-yl)-4-oxo-1,4-dihydropyridazin-3-yl]methyl}phenyl)carbamate.

LRMS (ESI) calc'd for C19H21F2N6O3 [M+H]⁺: 419. Found: 419.

The following examples were prepared from Intermediates #158-164, 166-172 according to Scheme 12 following a similar procedure described for Example #614 which can be achieved by

| | | | Exact |
|---|---|---|---|
| | | | Mass |
| Example | Structure | IUPAC Name | [M + H]⁺ |
| 615 | 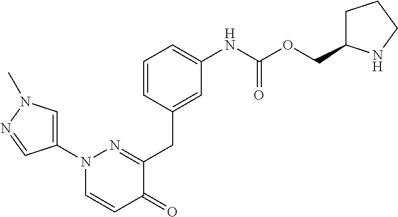 | (2R)-pyrrolidin- 2-ylmethyl (3- {[1-(1-methyl- 1H-pyrazol-4- yl)-4-oxo-1,4- dihydropyridazin- 3- yl]methyl} phenyl)carbamate | Calc'd 409, found 409 |
| 616 | 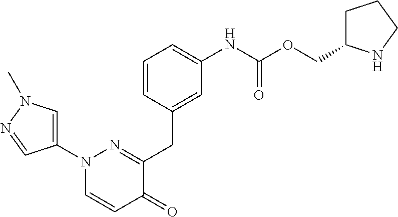 | (2S)-pyrrolidin- 2-ylmethyl (3- {[1-(1-methyl- 1H-pyrazol-4- yl)-4-oxo-1,4- dihydropyridazin- 3- yl]methyl}phenyl) carbamate | Calc'd 409, found 409 |
| 617 | 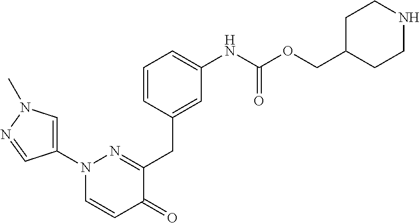 | piperidin-4- ylmethyl (3-{[1- (1-methyl-1H- pyrazol-4-yl)-4- oxo-1,4- dihydropyridazin- 3- yl]methyl} phenyl)carbamate | Calc'd 423, found 423 |
| 618 | 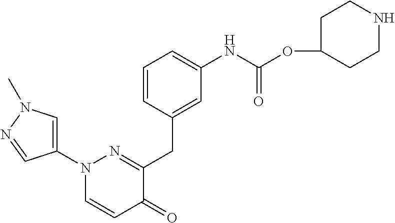 | piperidin-4-yl (3-{[1-(1- methyl-1H- pyrazol-4-yl)-4- oxo-1,4- dihydropyridazin-3- yl]methyl}phenyl) carbamate | Calc'd 409, found 409 |
| 619 | 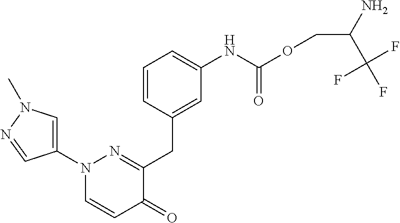 | rac-2-amino- 3,3,3- trifluoropropyl (3-{[1-(1- methyl-1H- pyrazol-4-yl)-4- oxo-1,4- dihydropyridazin- 3- yl]methyl}phenyl) carbamate | Calc'd 437, found 437 |
| 620 | 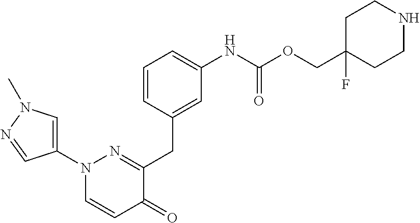 | (4- fluoropiperidin- 4-yl)methyl (3- {[1-(1-methyl- 1H-pyrazol-4- yl)-4-oxo-1,4- dihydropyridazin- 3- yl]methyl}phenyl) carbamate | Calc'd 441, found 441 |
| 621 | 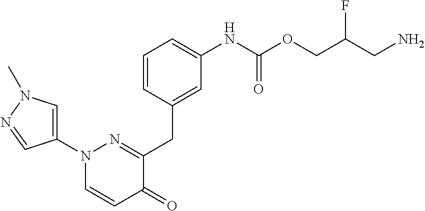 | rac-3-amino-2- fluoropropyl (3- {[1-(1-methyl- 1H-pyrazol-4- yl)-4-oxo-1,4- dihydropyridazin- 3- yl]methyl}phenyl) carbamate | Calc'd 401, found 401 |
| 622 | 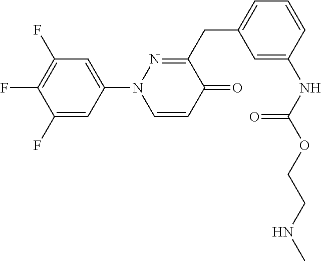 | 2- (methylamino) ethyl (3-{[4-oxo- 1-(3,4,5- trifluorophenyl)- 1,4- dihydropyridazin- 3- yl]methyl}phenyl) carbamate | Calc'd 433, found 433 |
| 623 | 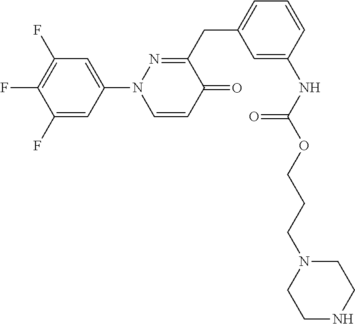 | 3-piperazin-1- ylpropyl (3-{[4- oxo-1-(3,4,5- trifluorophenyl)- 1,4- dihydropyridazin- 3- yl]methyl}phenyl) carbamate | Calc'd 502, found 502 |
| 624 | 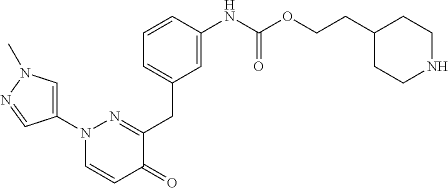 | 2-piperidin-4- ylethyl (3-{[1- (1-methyl-1H- pyrazol-4-yl)-4- oxo-1,4- dihydropyridazin- 3- yl]methyl}phenyl) carbamate | Calc'd 437, found 437 |
| 625 | 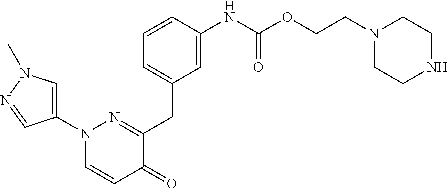 | 2-piperazin-1- ylethyl (3-{[1- (1-methyl-1H- pyrazol-4-yl)-4- oxo-1,4- dihydropyridazin- 3- yl]methyl}phenyl) carbmate | Calc'd 438, found 438 |
| 626 | 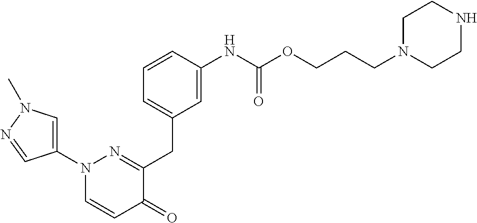 | 3-piperazin-1- ylpropyl (3-{[1- (1-methyl-1H- pyrazol-4-yl)-4- oxo-1,4- dihydropyridazin- 3- yl]methyl}phenyl) carbamate | Calc'd 452, found 452 |
| 627 | 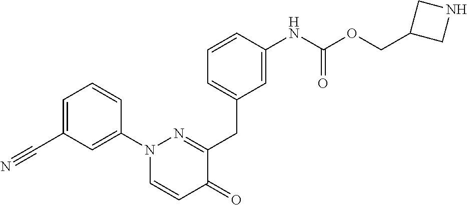 | azetidin-3- ylmethyl (3-{[1- (3- cyanophenyl)-4- oxo-1,4- dihydropyridazin- 3- yl]methyl}phenyl) carbamate | Calc'd 416, found 416 |
| 628 | 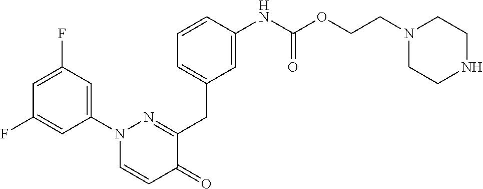 | 2-piperazin-1- ylethyl (3-{[1- (3,5- difluorophenyl)- 4-oxo-1,4- dihydropyridazin- 3- yl]methyl}phenyl) carbamate | Calc'd 470, found 470 |

### Scheme 12

### Example #629

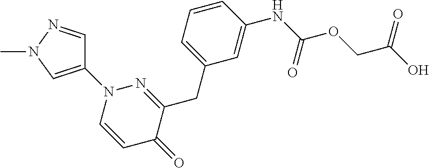

### ({[(3-{[1-(1-Methyl-1H-pyrazol-4-yl)-4-oxo-1,4-dihydropyridazin-3-yl]methyl}phenyl)amino]carbonyl}oxy)acetic acid

### Step 1 ({[(3-{[1-(1-Methyl-1H-pyrazol-4-yl)-4-oxo-1,4-dihydropyridazin-3-yl]methyl}phenyl)amino]carbonyl}oxy)acetic acid

Methyl ({[(3-{[1-(1-methyl-1H-pyrazol-4-yl)-4-oxo-1,4-dihydropyridazin-3-yl]methyl}phenyl)amino]carbonyl}oxy)acetate (Intermediate #173, 26 mg, 0.065 mmol) and 1 N lithium hydroxide (131 μL, 0.131 mmol) were stirred in THF (1.3 mL) at room temperature for 4 hours. 0.2 mL of 1 N HCl was added and the solvent removed in vacuo. Purification of the residue by reverse phase preparative HPLC (20-50% MeCN—H₂O, 0.1% TFA) gave ({[(3-{[1-(1-methyl-1H-pyrazol-4-yl)-4-oxo-1,4-dihydropyridazin-3-yl]methyl}phenyl)amino]carbonyl}oxy)acetic acid as an orange solid.

LRMS (ESI) calc'd for C18H18N5O5 [M+H]⁺: 384. Found: 384.

### Scheme 12

### Example #630

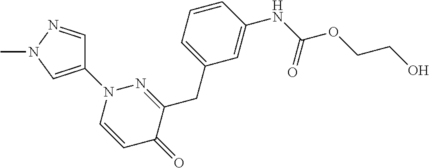

### 2-Hydroxyethyl(3-{[1-(1-methyl-1H-pyrazol-4-yl)-4-oxo-1,4-dihydropyridazin-3-yl]methyl}phenyl)carbamate

### Step 1. 2-Hydroxyethyl (3-{[1-(1-methyl-1H-pyrazol-4-yl)-4-oxo-1,4-dihydropyridazin-3-yl]methyl}phenyl)carbamate

2-{[tert-Butyl(dimethyl)silyl]oxy}ethyl(3-{[1-(1-methyl-1H-pyrazol-4-yl)-4-oxo-1,4-dihydropyridazin-3-yl]methyl}phenyl)carbamate (Intermediate #174, 48 mg, 0.098 mmol) was stirred in 1% cone. HCl in EtOH (1 mL) at room temperature for 3 hours. The solvent was removed in vacuo, saturated NaHCO₃ was added and the products extracted into 10% MeOH-DCM (x3). The combined organic extracts were dried over Na₂SO₄ and concentrated in vacuo to give 2-hydroxyethyl(3-{[1-(1-methyl-1H-pyrazol-4-yl)-4-oxo-1,4-dihydropyridazin-3-yl]methyl}phenyl)carbamate as a yellow solid.

LRMS (ESI)-calc'd for C18H20N5O4 [M+H]⁺: 370. Found: 370.

### Scheme 12

### Example #631

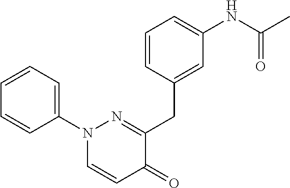

### N-{3-[(4-Oxo-1-phenyl-1,4-dihydropyridazin-3-yl)methyl]phenyl}acetamide

### Step 1. N-{3-[(4-Oxo-1-phenyl-1,4-dihydropyridazin-3-yl)methyl]phenyl}acetamide

3-(3-Aminobenzyl)-1-phenylpyridazin-4(1H)-one (Intermediate #157, 50 mg, 0.18 mmol) and acetyl chloride (13 μL, 0.18 mmol) were dissolved in THF (1.5 mL) and DIPEA (0.038 mL, 0.22 mmol) added. The mixture was stirred at room temperature. Upon completion, DCM was added and the reaction mixture was washed with sat. aqueous NaHCO₃ solution, followed by brine, dried over Na₂SO₄, filtered, and concentrated. The residue was purified by reverse phase preparative-HPLC (20-100% MeCN—H₂O, 0.05% TFA) to afford N-{3-[(4-Oxo-1-phenyl-1,4-dihydropyridazin-3-yl)methyl]phenyl}acetamide.

LRMS (ESI) calc'd for C18H18N3O3S [M+H]⁺: 355. Found: 355.

The following examples were prepared from Intermediate #132 according to Scheme 12 following a similar procedure described for Example #631, which can be achieved by those of ordinary skill in the art of organic synthesis.

| | | | Exact |
|---|---|---|---|
| | | | Mass |
| Example | Structure | IUPAC Name | [M + H]⁺ |
| 632 | 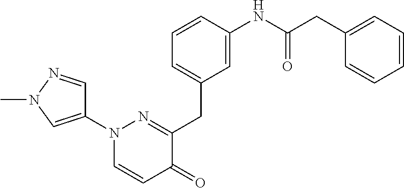 | N-(3-{[1-(1- methyl-1H- pyrazol-4-yl)-4- oxo-1,4- dihydropyridazin- 3- yl]methyl}phenyl)- 2- phenylacetamide | Calc'd 400, found 400 |
| 633 | 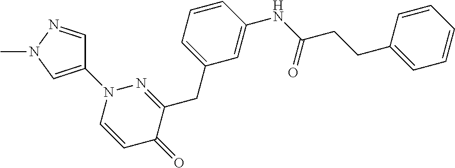 | N-(3-{[1-(1- methyl-1H- pyrazol-4-yl)-4- oxo-1,4- dihydropyridazin- 3- yl]methyl}phenyl)- 3- phenylpropanamide | Calc'd 414, found 414 |
| 634 | 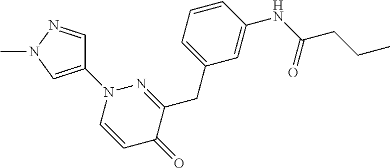 | N-(3-{[1-(1- methyl-1H- pyrazol-4-yl)-4- oxo-1,4- dihydropyridazin- 3- yl]methyl}phenyl) butanamide | Calc'd 352, found 352 |
| 635 | 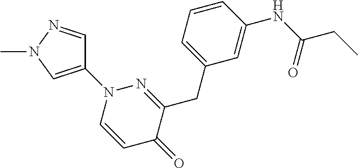 | N-(3-{[1-(1- methyl-1H- pyrazol-4-yl)-4- oxo-1,4- dihydropyridazin- 3- yl]methyl}phenyl) propanamide | Calc'd 338, found 338 |

### Scheme 12

### Example #636

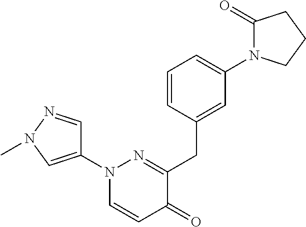

### 1-(1-Methyl-1H-pyrazol-4-yl)-3-[3-(2-oxopyrrolidin-1-yl)benzyl]pyridazin-4(1H)-one

### Step 1. 1-(1-Methyl-1H-pyrazol-4-yl)-3-[3-(2-oxopyrrolidin-1-yl)benzyl]pyridazin-4(1H)-one

A mixture of 3-(3-aminobenzyl)-1-(1-methyl-1H-pyrazol-4-yl)pyridazin-4(1H)-one (Intermediate #132, 50-mg, 0.18 mmol) and Et₃N (0.074 mL, 0.53 mmol) in DCM (1 mL) was treated with 4-bromobutyryl chloride (0.023 mL, 0.20 mmol) and stirred at r.t. for 24 hrs. Additional Et₃N (0.037 mL, 0.27 mmol) and 4-bromobutyryl chloride (0.012, 0.098 mmol) were added and the reaction mixture was stirred for an additional 24 hrs. Upon completion, DCM was added and the reaction mixture was washed with sat. NaHCO₃ solution, brine, and the combined organic phases were dried over Na₂SO₄, filtered, and concentrated. The residue was purified by reverse phase preparative HPLC (0-100% MeCN—H₂O, 0.05% TFA) to afford 1-(1-methyl-1H-pyrazol-4-yl)-3-[3-(2-oxopyrrolidin-1-yl)benzyl]pyridazin-4(1H)-one.

LRMS (ESI) calc'd for C19H20N5O2 [M+H]⁺: 350. Found: 350.

### Scheme 12

### Example #637

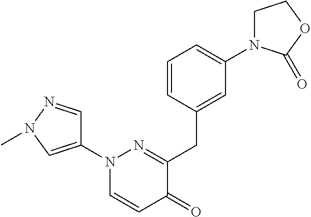

### 1-(1-Methyl-1H-pyrazol-4-yl)-3-[3-(2-oxo-1,3-oxazolidin-3-yl)benzyl]pyridazin-4(1H)-one

### Step 1. 1-(1-Methyl-1H-pyrazol-4-yl)-3-[3-(2-oxo-1,3-oxazolidin-3-yl)benzyl]pyridazin-4(1H)-one

To a stirred mixture of K₂CO₃ (61 mg, 0.44 mmol) and 3-(3-aminobenzyl)-1-(1-methyl-1H-pyrazol-4-yl)pyridazin-4(1H)-one (Intermediate #132, 50 mg, 0.18 mmol) in MeCN (1 mL) under an Ar atmosphere was added 2-chloroethyl chloroformate (0.028-mL, 0.27 mmol) at r.t. and the reaction was stirred for 1 h. The reaction was heated to reflux overnight. Additional 5 equivalents of K₂CO₃ and 2-chloroethyl chloroformate were added and the reaction mixture was refluxed for a further 24 hr. Upon completion, the reaction mixture was cooled and partitioned between EtOAc and H₂O. The aqueous layer was extracted with EtOAc and the combined organic layers washed with brine, dried over Na₂SO₄, filtered, and concentrated in vacuo. The residue was purified by reverse phase preparative HPLC (10-100% MeCN—H₂O, 0.05% TFA) followed by flash chromatography (MPLC, 0-10% MeOH-DCM) to afford 1-(1-methyl-1H-pyrazol-4-yl)-3-[3-(2-oxo-1,3-oxazolidin-3-yl)benzyl]pyridazin-4(1H) one.

LRMS (ESI) calc'd for C18H18N5O3 [M+H]⁺: 352. Found: 352.

### Scheme 13

### Example #638

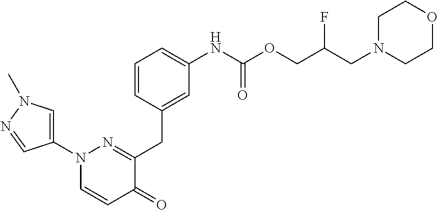

### rac-2-Fluoro-3-morpholin-4-ylpropyl (3-{[1-(1-methyl-1H-pyrazol-4-yl)-4-oxo-1,4-dihydropyridazin-3-yl]methyl}phenyl)carbamate

### Step 1. rac-2-Fluoro-3-morpholin-4-ylpropyl (3-{[1-(1-methyl-1H-pyrazol-4-yl)-4-oxo-1,4-dihydropyridazin-3-yl]methyl}phenyl)carbamate

rac-3-Amino-2-fluoropropyl (3-{[1-(1-methyl-1H-pyrazol-4-yl)-4-oxo-1,4-dihydropyridazin-3-yl]methyl}phenyl)carbamate (Example #621, 75 mg, 0.17 mmol), bis(2-bromoethyl)ether (44 mg, 0.19 mmol) and DIPEA (0.120 mL, 0.687 mmol) were dissolved in DMF (1 mL) and stirred at 65° C. for 12 hrs. Additional bis(2-bromoethyl)ether (43.8 mg, 0.189 mmol) and DIPEA (0.120 mL, 0.687 mmol) were added and the reaction was heated at 65° C. for 6 hours. After cooling to room temperature, the reaction mixture was diluted with EtOAc and washed with brine. The organic layer was dried over Na₂SO₄, filtered and concentrated in vacuo. The residue was purified by flash chromatography (MPLC, 0-20% MeOH-EtOAc) to give rac-2-fluoro-3-morpholin-4-ylpropyl (3-{[1-(1-methyl-1H-pyrazol-4-yl)-4-oxo-1,4-dihydropyridazin-3-yl]methyl}phenyl)carbamate as a white solid.

LRMS (ESI) calc'd for C23H28FN6O4 [M+H]⁺: 471. Found: 471.

### Scheme 14

### Example #639

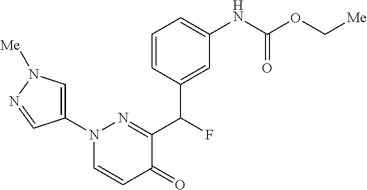

### rac-Ethyl(3-{fluoro[1-(1-methyl-1H-pyrazol-4-yl)-4-oxo-1,4-dihydropyridazin-3-yl]methyl}phenyl)carbamate

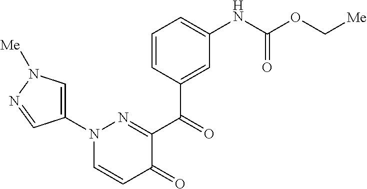

### Step 1. Ethyl(3-{[1-(1-methyl-1H-pyrazol-4-yl)-4-oxo-1,4-dihydropyridazin-3-yl]carbonyl}phenyl)carbamate

3-(3-Aminobenzoyl)-1-(1-methyl-1H-pyrazol-4-yl)pyridazin-4(1H)-one (Intermediate #132, Alternative Aniline Synthesis Step 2, 134 mg, 0.454 mmol) was suspended in THF (4.5 mL) and DIPEA (0.095 mL, 0.55 mmol) was added followed by ethyl chloroformate (0.048 mL, 0.50 mmol). The resulting mixture was stirred at r.t. for 1 hour. Additional THF (4.5 mL) was added and stirring at r.t. continued for 1 hour. Additional DIPEA (0.019 mL, 0.11 mmol) and ethyl chloroformate (9.6 μL, 0.10 mmol) were added and stirring at r.t. continued for 1 hour. Additional THF (4.5 mL) was added and the reaction was warmed to 55° C. for 3 hours then stirred at r.t. overnight. MeOH was added and the solvent removed in vacuo while loading onto silica. Purification of the residue by flash chromatography (MPLC, 0-10% MeOH-DCM) gave ethyl(3-{[1-(1-methyl-1H-pyrazol-4-yl)-4-oxo-1,4-dihydropyridazin-3-yl]carbonyl}phenyl)carbamate as a yellow solid.

LRMS (ESI) calc'd for C18H18N5O4 [M+H]⁺: 368. found: 368.

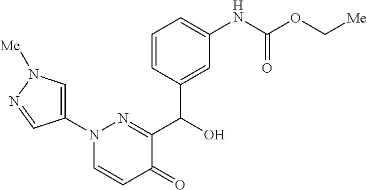

### Step 2. rac-Ethyl(3-{hydroxy[1-(1-methyl-1H-pyrazol-4-yl)-4-oxo-1,4-dihydropyridazin-3-yl]methyl}phenyl)carbamate

Ethyl(3-{[1-(1-methyl-1H-pyrazol-4-yl)-4-yl)-4-oxo-1,4-dihydropyridazin-3-yl]carbonyl}phenyl)carbamate (144 mg, 0.39 mmol) and NaBH₄ (16 mg, 0.43 mmol) were stirred in MeOH (8 mL) at r.t. overnight. Water was added and the solvent removed in vacuo while loading onto silica. Purification of the residue by flash chromatography (MPLC, 0-15% MeOH-DCM) afforded ethyl(3-{hydroxy[1-(1-methyl-1H-pyrazol-4-yl)-4-oxo-1,4-dihydropyridazin-3-yl]methyl}phenyl)carbamate as a pale yellow solid.

LRMS (ESI) calc'd for C18H20N5O4 [M+H]⁺: 370. found: 370.

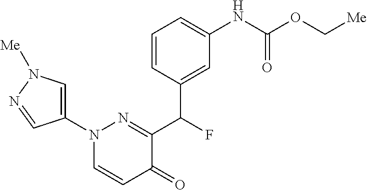

### Step 3. rac-Ethyl(3-{hydroxy[1-(1-methyl-1H-pyrazol-4-yl)-4-oxo-1,4-dihydropyridazin-3-yl]methyl}phenyl)carbamate

rac-Ethyl(3-{hydroxy[1-(1-methyl-1H-pyrazol-4-yl)-4-oxo-1,4-dihydropyridazin-3-yl]methyl}phenyl)carbamate (81 mg, 0.22 mmol) and DAST (0.032 mL, 0.24 mmol) were stirred in DCM (4 mL) at r.t. for 2 hours. Additional DAST (0.032 mL, 0.24 mmol) was added and stirring at r.t. continued for 1 hour. Si-carbonate was added followed by MeOH and the solvent was removed in vacuo. Purification of the residue by flash chromatography (MPLC, 0-8% MeOH-DCM) afforded ethyl(3-{fluoro[1-(1-methyl-1H-pyrazol-4-yl)-4-oxo-1,4-dihydropyridazin-3-yl]methyl}phenyl)carbamate as a pale yellow solid.

LRMS (ESI) calc'd for C18H19FN5O3 [M+H]⁺: 372. found: 372.

### Scheme 14

### Example #640

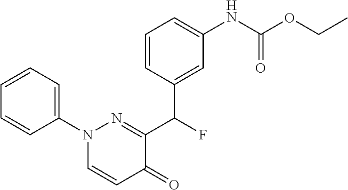

### Ethyl{3-[flouro(4-oxo-1-phenyl-1,4-dihydropyridazin-3-yl)methyl]phenyl}carbamate

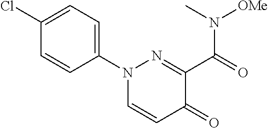

### Step 1. 1-(4-Chlorophenyl)-N-methoxy-N-methyl-4-oxo-1,4-dihydropyridazine-3-carboxamide

1-(4-Chlorophenyl)-N-methoxy-N-methyl-4-oxo-1,4-dihydropyridazine-3-carboxamide was prepared from 1-(4-chlorophenyl)-4-oxo-1,4-dihydropyridazine-3-carboxylic acid according to the procedure described for N-methoxy-N-methyl-1-(1-methyl-1H-pyrazol-4-yl)-4-oxo-1,4-dihydropyridazine-3-carboxamide (Intermediate #132 Alternative Aniline Synthesis Step 1).

LRMS (ESI) calc'd for C13H13ClN3O3 [M+H]⁺: 294. found: 294.

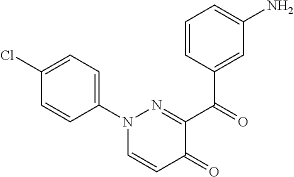

### Step 2. 3-(3-Aminobenzoyl)-1-(4-chlorophenyl)pyridazin-4(1H)-one

3-(3-Aminobenzoyl)-1-(4-chlorophenyl)pyridazin-4(1H)-one was prepared from 1-(4-chlorophenyl)-N-methoxy-N-methyl-4-oxo-1,4-dihydropyridazine-3-carboxamide according to the procedure described for 3-[(3-aminophenyl)carbonyl]-1-(1-methyl-1H-pyrazol-4-yl)pyridazin-4(1H)-one (Intermediate #132, Alternative Aniline Synthesis Step 2).

LRMS (ESI) calc'd for C17H12ClN3O2 [M+H]⁺: 326. found: 326.

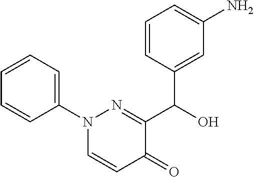

### Step 3. rac-3-[(3-Aminophenyl)(hydroxy)methyl]-1-phenylpyridazin-4(1H)-one

3-[(3-Aminophenyl)carbonyl]-1-(4-chlorophenyl)pyridazin-4(1H)-one (80 mg, 0.25 mmol) and Pd/C (10 wt %, 5 mg, 4.9 μmol) were stirred in MeOH (2.5 mL)/DMA (2.5 mL) under H₂ (1 atm) overnight. The catalyst was removed by filtration through Celite and the solvent removed in vacuo. Purification of the residue by flash chromatography (MPLC, 40-100% EtOAc-hexanes followed by 0-10% MeOH-EtOAc) gave a ˜1:1 mixture of rac-3-[(3-aminophenyl)(hydroxy)methyl]-1-phenylpyridazin-4(1H)-one and 3-(3-aminobenzyl)-1-phenylpyridazin-4(1H)-one as a yellow gum.

LRMS (ESI) calc'd for C17H16N3O2 [M+H]⁺: 294. found: 294.

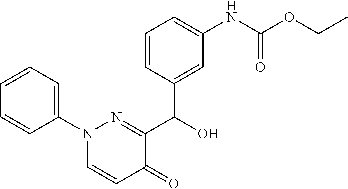

### Step 4. rac-Ethyl-{3-[hydroxy(4-oxo-1-phenyl-1,4-dihydropyridazin-3-yl)methyl]phenyl}carbamate

A mixture of rac-3-[(3-aminophenyl)(hydroxy)methyl]-1-phenylpyridazin-4(1H)-one (25 mg, 0.085 mmol) and 3-(3-aminobenzyl)-1-phenylpyridazin-4(1H)-one (24 mg, 0.085 mmol) was taken up in THF (2 mL) and DIPEA (0.033 mL, 0.19 mmol) was added followed by ethyl chloroformate (0.017 mL, 0.18 mmol). The resulting mixture was stirred at r.t. overnight. The sol-vent was removed in vacuo and the residue purified by reverse phase preparative HPLC (20-80% MeCN—H₂O, 0.05% TFA) to give rac-ethyl{3-[hydroxy(4-oxo-1-phenyl-1,4-dihydropyridazin-3-yl)methyl]phenyl}carbamate as a yellow solid and ethyl {3-[(4-oxo-1-phenyl-1,4-dihydropyridazin-3-yl)methyl]phenyl}carbamate as a beige solid.

LRMS (ESI) calc'd for C21H24N3O4 [M+H]⁺: 382. found: 382.

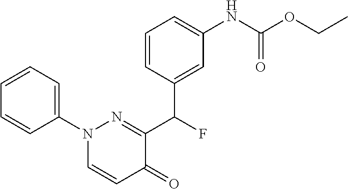

### Step 5. rac-Ethyl-{3-[fluoro(4-oxo-1-phenyl-1,4-dihydropyridazin-3-yl)methyl]phenyl}carbamate

rac-Ethyl-{3-[hydroxy(4-oxo-1-phenyl-1,4-dihydropyridazin-3-yl)methyl]phenyl}carbamate (19 mg, 0.052 mmol) and DAST (8 μL, 0.06 mmol) were stirred in DCM (1 mL) at r.t. for 2 hours. Additional DAST 8 μL, 0.06 mmol) was added and stirring at r.t. continued for 90 minutes. Si-carbonate was added, followed by MeOH and the solvent removed in vacuo. Purification of the residue by flash chromatography (MPLC, 40-100% EtOAc-Hexanes) gave rac-ethyl{3-[fluoro(4-oxo-1-phenyl-1,4-dihydropyridazin-3-yl)methyl]phenyl}carbamate as a white solid.

LRMS (ESI) calc'd for C20H19FN3O3 [M+H]⁺: 368. found: 368.

### Scheme 14

### Example #641

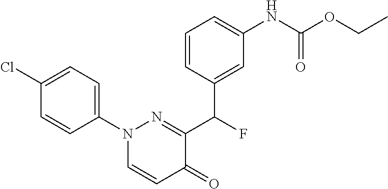

### rac-Ethyl(3-{[1-(4-chlorophenyl)-4-oxo-1,4-dihydropyridazin-3-yl](fluoro)methyl}phenyl)carbamate

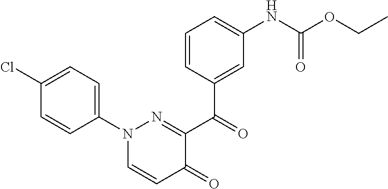

### Step 1. Ethyl 3-{[1-(4-chlorophenyl)-4-oxo-1,4-dihydropyridazin-3-yl]carbonyl}phenyl)carbamate

3-(3-Aminobenzoyl)-1-(4-chlorophenyl)pyridazin-4(1H)-one (Example #640 Step 2, 185 mg, 0.568 mmol) and DIPEA (0.119 mL, 0.682 mmol) were taken up in 1,4-dioxane (6 mL). Ethyl chloroformate (0.060 mL, 0.63 mmol) was added and the resulting suspension was stirred at room temperature for 4-hrs. 1 NHC was added and the aqueous phase was extracted with EtOAc (2×), the combined organic extracts were washed with brine, dried over Na₂SO₄, and concentrated in vacuo to give ethyl(3-{[1-(4-chlorophenyl)-4-oxo-1,4-dihydropyridazin-3-yl]carbonyl}phenyl)carbamate as a pale orange solid.

LRMS (ESI) calc'd for C20H17ClN3O4 [M+H]⁺: 398. found: 398.

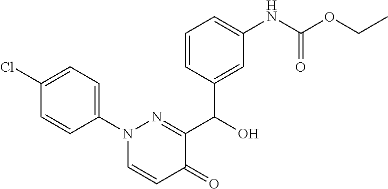

### Step 2. rac-Ethyl(3-{[1-(4-chlorophenyl)-4-oxo-1,4-dihydropyridazin-3-yl](hydroxy)methyl}phenyl)carbamate

Ethyl(3-{[1-(4-chlorophenyl)-4-oxo-1,4-dihydropyridazin-3-yl]carbonyl}phenyl)carbamate (214 mg, 0.538 mmol) was suspended in MeOH (6 mL) and NaBH₄ (22 mg, 0.59 mmol) was added. The resulting mixture was stirred at room temperature for 3 hours and additional NaBH₄ (6 mg, 0.16 mmol) was added and stirring at room temperature continued for 1 hour. 1 N HCl was added, the aqueous phase was extracted with EtOAc (2×), the combined-organic extracts were washed with brine, dried over Na₂SO₄, and concentrated in vacuo. The residue was triturated in EtOH to give rac-ethyl{3-[[1-(4-chlorophenyl)-4-oxo-1,4-dihydropyridazin-3-yl](hydroxy)methyl]phenyl}carbamate as a pale yellow solid.

LRMS (ESI) calc'd for C20H19ClN3O4 [M+H]⁺: 400. found: 400.

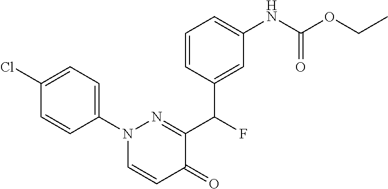

### Step 3. rac-Ethyl(3-{[1-(4-chlorophenyl-4-oxo-1,4-dihydropyridazin-3-yl](fluoro)methyl}phenyl)carbamate

rac-Ethyl{3-[[1-(4-chlorophenyl)-4-oxo-1,4-dihydropyridazin-3-yl](hydroxy)methyl]phenyl}carbamate (100 mg, 0.250 mmol) was suspended in DCM (2.5 mL) and DAST (0.036-mL, 0.275 mmol) was added. The resulting mixture was stirred at room temperature for 2 hrs. Saturated NaHCO₃ was added and the aqueous layer was extracted with EtOAc (2×). The combined organic extracts were washed with brine, dried over Na₂SO₄, concentrated in vacuo, and the residue was purified by flash chromatography (MPLC, 25-100% EtOAc-hexanes) to afford rac-ethyl{3-[[1-(4-chlorophenyl)-4-oxo-1,4-dihydropyridazin-3-yl](fluoro)methyl]phenyl}carbamate as a white solid.

LRMS (ESI) calc'd for C20H18ClFN3O3 [M+H]⁺: 402. found: 402.

### Scheme 14

### Example #642

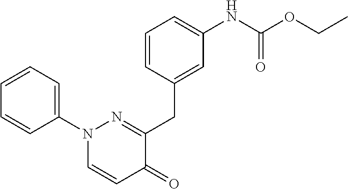

### Ethyl {3-[(4-oxo-1-phenyl-1,4-dihydropyridazin-3-yl)methyl]phenyl}carbamate

### Step 1. Ethyl-{3-[(4-oxo-1-phenyl-1,4-dihydropyridazin-3-yl)methyl]phenyl}carbamate

rac-Ethyl {3-[[1-(4-chlorophenyl)-4-oxo-1,4-dihydropyridazin-3-yl](fluoro)methyl]phenyl}carbamate (Example #641, 33 mg, 0.082 mmol) was taken up in MeOH (2 mL) and Pd/C (10 wt %, 1.7 mg, 1.6 μmol) was added. The resulting suspension was stirred under H₂ (1 atm) at r.t. for 3 hours. DMA (1 mL) was added to aid solubility and stirring continued for 3 hours. Additional DMA (1 mL) was added and stirring continued overnight. Additional Pd/C (10 wt %, 2 mg, 2 μmol) was added and stirring continued for 24 hours. The catalyst was removed by filtering through Celite, the solvent removed in vacuo, and the residue was purified by flash chromatography (MPLC, 5-100% EtOAc-Hexanes) to give ethyl {3-[(4-oxo-1-phenyl-1,4-dihydropyridazin-3-yl)methyl]phenyl}carbamate as a white solid.

LRMS (ESI) calc'd for C20H20N₃O3 [M+H]⁺: 350. found: 350.

### Scheme 14

### Example #643

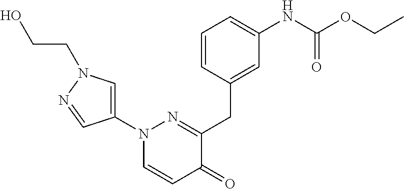

### Ethyl [3-({1-[1-(2-hydroxyethyl)-1H-pyrazol-4-yl]-4-oxo-1,4-dihydropyridazin-3-yl}methyl)phenyl]carbamate

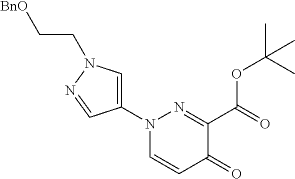

### Step 1. tert-Butyl 1-{1-[2-(benzyloxy)ethyl]-1H-pyrazol-4-yl}-4-oxo-1,4-dihydropyridazine-3-carboxylate

tert-Butyl 1-{1-[2-(benzyloxy)ethyl]-1H-pyrazol-4-yl}-4-oxo-1,4-dihydropyridazine-3-carboxylate was prepared from tert-butyl 4-oxo-1-(1H-pyrazol-4-yl)-1,4-dihydropyridazine-3-carboxylate (Intermediate #27 Step 2) according to the procedure described for tert-butyl 4-oxo-1-[1-(propan-2-yl)-1H-pyrazol-4-yl]-1,4-dihydropyridazine-3-carboxylate (Intermediate #27 Step 3).

LRMS (ESI) calc'd for C21H25N4O4 [M+H]⁺: 397. Found: 397.

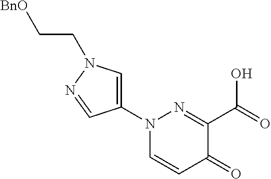

### Step 2. 1-{1-[2-(Benzyloxy)ethyl]-1H-pyrazol-4-yl}-4-oxo-1,4-dihydropyridazine-3-carboxylic acid

1-{1-[2-(Benzyloxy)ethyl]-1H-pyrazol-4-yl}-4-oxo-1,4 dihydropyridazine-3-carboxylic acid was prepared from tert-butyl 1-{1-[2-(benzyloxy)ethyl]-1H-pyrazol-4-yl}-4-oxo-1,4-dihydropyridazine-3-carboxylate according to the procedure described for 4-oxo-1-[1-(propan-2-yl)-1H-pyrazol-4-yl]-1,4-dihydropyridazine-3-carboxylic-acid (Intermediate #27 Step 4).

LRMS (ESI) calc'd for C17H17N4O4 [M+H]⁺: 341. Found: 341.

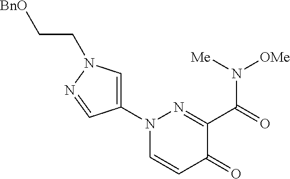

### Step 3. 1-(1-(2-(Benzyloxyethyl)-1H-pyrazol-4-yl-N-methoxy-N-methyl-4-oxo-1,4 dihydropyridazine-3-carboxamide

1-(1-(2-(Benzyloxy)ethyl)-1H-pyrazol-4-yl)-N-methoxy-N-methyl-4-oxo-1,4-dihydropyridazine-3-carboxamide was prepared from 1-{1-[2-(benzyloxy)ethyl]-1H-pyrazol-4-yl}-4-oxo-1,4 dihydropyridazine-3-carboxylic acid according to the procedure described for N-methoxy-N-methyl-1-(1-methyl-1H-pyrazol-4-yl)-4-oxo-1,4-dihydropyridazine-3-carboxamide (Intermediate #132 Alternative Aniline Synthesis Step 1).

LRMS (ESI) calc'd for C19H21N5O4 [M+H]⁺: 384. Found: 384.

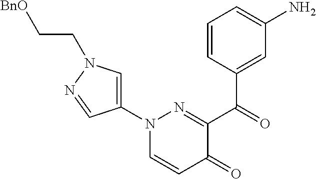

### Step 4. 3-[(3-Aminophenyl)carbonyl]-1-{1-[2-(benzyloxy)ethyl]-1-pyrazol-4-yl}pyridazin-4(1H)-one

3-(3-Aminobenzoyl)-1-(1-(2-(benzyloxy)ethyl)-1H-pyrazol-4-yl)pyridazin-4(1H)-one was prepared from 1-(1-(2-(benzyloxy)ethyl)-1H-pyrazol-4-yl)-N-methoxy-N-methyl-4-oxo-1,4-dihydropyridazine-3-carboxamide according to the procedure described for 3-(3-aminobenzyl)-1-(1-methyl-1H-pyrazol-4-yl)pyridazin-4(1H)-one (Intermediate #132, Alternative Aniline Synthesis Step 2).

LRMS (ESI) calc'd for C23H22N5O3 [M+H]⁺: 416. Found: 416.

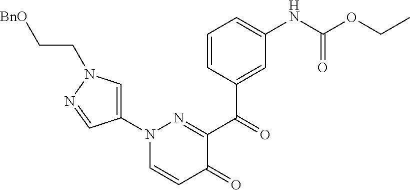

### Step 5. Ethyl {3-[(1-{1-[2-(benzyloxy)ethyl]-1H-pyrazol-4-yl}-4-oxo-1,4-dihydropyridazin-3-yl)carbonyl]phenyl}carbamate

3-(3-Aminobenzoyl)-1-(1-(2-(benzyloxy)ethyl)-1H-pyrazol-4-yl)pyridazin-4(1H)-one (1.36 g, 3.27 mmol) was suspended in THF (31 mL), and DIPEA (0.743 mL, 3.93 mmol) was added followed by ethyl chloroformate (0.377 mL, 3.93 mmol) and the reaction was stirred at room temperature for 1 h. MeOH was added, the solvent was removed in vacuo and the residue was purified by flash chromatography (MPLC, 0-15% MeOH-DCM) to afford ethyl {3-[(1-{1-[2-(benzyloxy)ethyl]-1H-pyrazol-4-yl}-4-oxo-1,4-dihydropyridazin-3-yl)carbonyl]phenyl}carbamate.

LRMS (APCI) calc'd for C26H25N5O5 [M+H]⁺: 488. Found: 488.

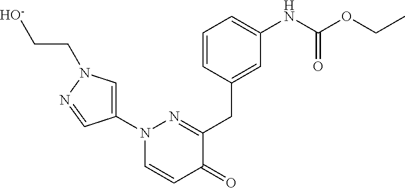

### Step 6. Ethyl [3-({1-[1-(2-hydroxyethyl)-1H-pyrazol-4-yl]-4-oxo-1,4-dihydropyridazin-3-yl}methyl)phenyl]carbamate

To a suspension of Pd(OH)₂ (10 wt %, 70 mg) in MeOH (35 mL) in a Parr flask was added ethyl 3-(1-(1-(2-(benzyloxy)ethyl)-1H-pyrazol-4-yl)-4-oxo-1,4-dihydropyridazine-3-carbonyl)phenylcarbamate (700 mg, 1.43 mmol) in EtOAc (8.7 mL). The suspension was agitated under H₂ (40 psi) for 24 hr. Additional Pd(OH)₂ (10 wt %, 70 mg) was added and the reaction mixture was agitated under H₂ (40 psi) for a further 24 hrs. The addition was repeated after 12 hours. Upon completion, the catalyst was removed by filtering through Celite, the solvent was evaporated and the residue was purified by flash chromatography (MPLC; 0-15% MeOH-DCM) to provide ethyl [3-({1-[1-(2-hydroxyethyl)-1H-pyrazol-4-yl]-4-oxo-1,4-dihydropyridazin-3-yl}methyl)phenyl]carbamate.

LRMS (ESI) calc'd for C19H22N5O4 [M+H]⁺: 384. Found: 384.

The following examples were prepared according to Scheme 14 following similar procedures described for Example #643, which can be achieved by those of ordinary skill in the art of organic synthesis.

| | | | Exact |
|---|---|---|---|
| | | | Mass |
| Example | Structure | IUPAC Name | [M + H]⁺ |
| 644 | 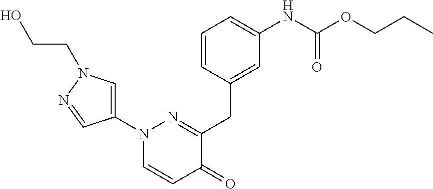 | propyl [3-({1-[1-(2- hydroxyethyl)-1H- pyrazol-4-yl]-4- oxo-1,4- dihydropyridazin-3- yl}methyl)phenyl] carbamate | Calc'd 398, found 398 |
| 645 | 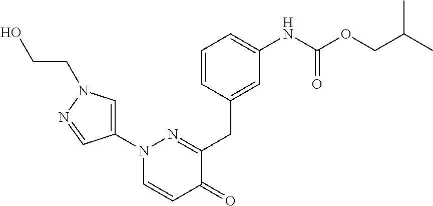 | 2-methylpropyl [3- ({1-[1-(2- hydroxyethyl)-1H- pyrazol-4-yl]-4- oxo-1,4- dihydropyridazin-3- yl}methyl)phenyl] carbamate | Calc'd 412, found 412 |

### Scheme 14

### Example #646

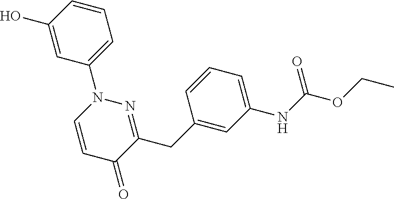

### Ethyl(3-{[1-(3-hydroxyphenyl)-4-oxo-1,4-dihydropyridazin-3-yl]methyl}phenyl)carbamate

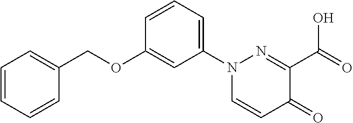

### Steps 1-3. 1-[3-(Benzyloxy)phenyl]-4-oxo-1,4-dihydropyridazine-3-carboxylic acid

1-[3-(Benzyloxy)phenyl]-4-oxo-1,4-dihydropyridazine-3-carboxylic acid was prepared from 3-(benzyloxy)aniline according to the procedures described for 4-oxo-1-[1-(propan-2-yl)-1H-pyrazol-4-yl]-1,4-dihydropyridazine-3-carboxylic acid (Intermediate #1 Steps 1-3).

LRMS (ESI) calc'd for C18H15N2O4 [M+H]⁺: 323. Found: 323.

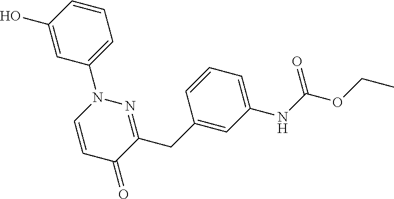

### Steps 4-7. Ethyl(3-{[1-(3-hydroxyphenyl)-4-oxo-1,4-dihydropyridazin-3-yl]methyl}phenyl)carbamate

Ethyl(3-{[1-(3-hydroxyphenyl)-4-oxo-1,4-dihydropyridazin-3-yl]methyl}phenyl)carbamate was prepared from 1-[3-(benzyloxy)phenyl]-4-oxo-1,4-dihydropyridazine-3-carboxylic acid according to the procedures described for ethyl [3-({1-[1-(2-hydroxyethyl)-1H-pyrazol-4-yl]-4-oxo-1,4-dihydropyridazin-3-yl}methyl)phenyl]carbamate (Example #643 Steps-3-6).

LRMS (ESI) calc'd for C20H20N3O4 [M+H]⁺: 366. Found: 366.

### Scheme 15

### Example #647

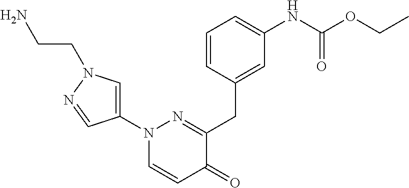

### Ethyl [3-({1-[1-(2-aminoethyl)-1H-pyrazol-4-yl]-4-oxo-1,4-dihydropyridazin-3-yl}methyl)phenyl]carbamate

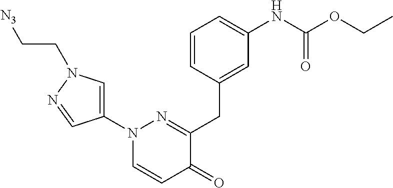

### Step 1. Ethyl[3-({1-[1-(2-azidoethyl)-H-pyrazol-4-yl]-4-oxo-1,4-dihydropyridazin-3-yl}methyl)phenyl]carbamate

To a solution of triphenylphosphine (44 mg, 0.17 mmol) in THF (1.2 mL) was added diisopropyl azodicarboxylate (38 μL, 0.20 mmol) at 0° C. and the solution was stirred for min. To this solution was added ethyl [3-({1-[1-(2-hydroxyethyl)-1H-pyrazol-4-yl]-4-oxo-1,4-dihydropyridazin-3-yl}methyl)phenyl]carbamate (Example #643, 50 mg, 0.13 mmol). After stirring for 10 min, diphenylphosphoryl azide (42 μL, 0.20 mmol) was added. The reaction mixture was allowed to stir at r.t. for 4 hours. Upon complete conversion, the solvent was evaporated and the residue was purified by flash chromatography (2×) (MPLC, 0-15% MeOH-DCM) to provide ethyl [3-({1-[1-(2-azidoethyl)-1H-pyrazol-4-yl]-4-oxo-1,4-dihydropyridazin-3-yl}methyl)phenyl]carbamate.

LRMS (ESI) calc'd for C19H21N8O3 [M+H]⁺: 409. Found: 409.

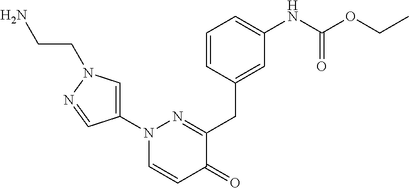

### Step 2. Ethyl [3-({1-[1-(2-aminoethyl)-1H-pyrazol-4-yl]-4-oxo-1,4-dihydropyridazin-3-yl}methyl)phenyl]carbamate

To a solution of ethyl [3-({1-[1-(2-azidoethyl)-1H-pyrazol-4-yl]-4-oxo-1,4-dihydropyridazin-3-yl}methyl)phenyl]carbamate (20 mg, 0.049 mmol) in EtOH (490 μL) was added Pd/C (10 wt %, 4 mg). The reaction was stirred under H₂ (1 atm) for 2 hours. Upon complete conversion, the reaction mixture was filtered through Celite and the solvent was evaporated to provide ethyl [3-({1-[1-(2-aminoethyl)-1H-pyrazol-4-yl]-4-oxo-1,4-dihydropyridazin-3-yl}methyl)phenyl]carbamate.

LRMS (ESI) calc'd for C19H23N6O3 [M+H]⁺: 383. Found: 383.

The following example was prepared from Example #645 according to Scheme 15 following similar procedures described for Example #647, which can be achieved by those of ordinary skill in the art of organic synthesis.

| | | | Exact |
|---|---|---|---|
| | | | Mass |
| Example | Structure | IUPAC Name | [M + H]⁺ |
| 648 | 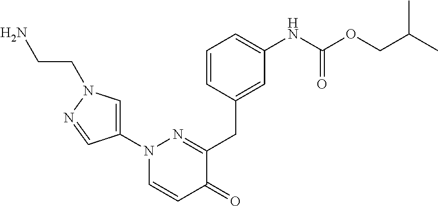 | 2-methylpropyl [3- ({1-[1-(2- aminoethyl)-1H- pyrazol-4-yl]-4- oxo-1,4- dihydropyridazin-3- yl}methyl)phenyl] carbamate | Calc'd 411, found 411 |

### Scheme 16

### Example #649

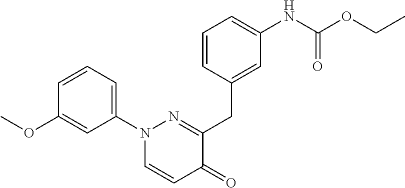

### Ethyl(3-{[1-(3-methoxyphenyl)-4-oxo-1,4-dihydropyridazin-3-yl]methyl}phenyl)carbamate

### Step 1. Ethyl(3-{[1-(3-methoxyphenl)-4-oxo-1,4-dihydropyridazin-3-yl]methyl}phenyl)carbamate

Ethyl(3-{[1-(3-hydroxyphenyl)-4-oxo-1,4-dihydropyridazin-3-yl]methyl}phenyl)carbamate (Example #646, 20 mg, 0.055 mmol), Cs₂CO₃ (21 mg, 0.066 mmol), and dimethyl sulfate (6 μL, 0.07 mmol) were dissolved in DMF (251 μL). The mixture was stirred at 50° C. Upon complete conversion, water was added, the mixture was extracted with EtOAc (3×), the combined organic phases were dried over Na₂SO₄, filtered and the solvent was evaporated. The residue was purified by reverse phase preparative HPLC (30-100% MeCN—H₂O, 0.05% TFA). After removal of the solvent in vacuo the free base was liberated by dissolving the residue in DCM/MeOH (4:1), filtering through PL-HCO₃ cartridges (Stratospheres™, 0.9 mmol) and concentrating in vacuo to provide ethyl(3-{[1-(3-methoxyphenyl)-4-oxo-1,4-dihydropyridazin-3-yl]methyl}phenyl)carbamate.

LRMS (ESI) calc'd for C21H22N3O4 [M+H]⁺: 380. Found: 380.

### Scheme #16

### Example #650

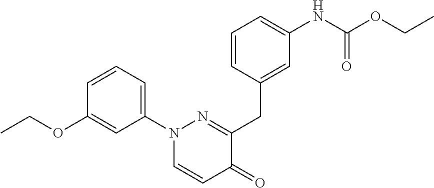

### Ethyl(3-{[1-(3-ethoxyphenyl)-4-oxo-1,4-dihydropyridazin-3-yl]methyl}phenyl)carbamate

### Step 1. Ethyl(3-{[1-(3-ethoxyphenyl)-4-oxo-1,4-dihydropyridazin-3-yl]methyl}phenyl)carbamate

Ethyl(3-{[1-(3-hydroxyphenyl)-4-oxo-1,4-dihydropyridazin-3-yl]methyl}phenyl)carbamate (Example #646, 25 mg, 0.068 mmol), Cs₂CO₃ (27 mg, 0.082 mmol) and bromoethane (4.9 μL, 0.066 mmol) were dissolved in DMF (314 μL). The mixture was stirred at 120° C. for 30 min under microwave irradiation (Biotage, Initiator). Water was added and the mixture was extracted with EtOAc (3×). The combined organic extracts were dried over Na₂SO₄, filtered and the solvent was evaporated. The residue was purified by flash chromatography (MPLC, 0-15% MeOH-DCM) and subsequently by reverse phase preparative HPLC (35-100% MeCN—H₂O, 0.05% TFA). After removal of the solvent in vacuo the free base was liberated by dissolving the residue in DCM/MeOH (4:1), filtering through PL-HCO₃ cartridges (Stratospheres™, 0.9 mmol) and concentrating in vacuo to afford ethyl 3-((1-(3-ethoxyphenyl)-4-oxo-1,4-dihydropyridazin-3-yl)methyl)phenylcarbamate.

LRMS (ESI) calc'd for C22H24N3O4 [M+H]⁺: 394. Found: 394.

### Scheme 17

### Example #651

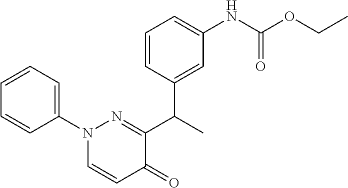

### rac-Ethyl {3-[1-(4-oxo-1-phenyl-1,4-dihydropyridazin-3-yl)ethyl]phenyl}carbamate

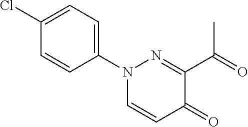

### Step 1. 3-Acetyl-1-(4-chlorophenyl)pyridazin-4(1H)-one

1-(4-Chlorophenyl)-N-methoxy-N-methyl-4-oxo-1,4-dihydropyridazine-3-carboxamide (Example #640 Step 1, 0.50 g, 1.7 mmol) was taken up in THF (3.8 mL) and cooled to −78° C. Methylmagnesium iodide (1.42 mL, 4.26 mmol) was added and stirring at −78° C. continued for 40 minutes. 2 N HCl was added followed by H₂O and the products extracted into EtOAc (2×). The combined organic extracts were washed with brine, dried over MgSO₄, and concentrated in vacuo. The product residue was triturated in Et₂O to give 3-acetyl-1-(4-chlorophenyl)pyridazin-4(1H)-one as an orange solid.

LRMS (ESI) calc'd for C12H10ClN2O2 [M+H]⁺: 249. found: 249.

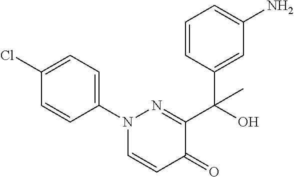

### Step 2., rac-3-[1-(3-Aminophenyl)-1-hydroxyethyl]-1-(4-chlorophenyl)pyridazin-4(1H)-one

3-Acetyl-1-(4-chlorophenyl)pyridazin-4(1H)-one (94 mg, 0.38 mmol) was taken up in THF (3.8 mL) and cooled to −78° C. {3-[Bis(trimethylsilyl)amino]phenyl}magnesium chloride (0.95 ml, 0.95 mmol) was added and stirring at −78° C. continued for 45 minutes. 2 N HCl. (0.9 mL) was added followed by brine and the aqueous layer was extracted with EtOAc (2×). The combined organic extracts were washed with brine, dried over MgSO₄, and concentrated in vacuo. The product solid was taken up in DCM (˜3 mL) and TFA (˜3 mL) was added. After 1-hour at room temperature, saturated NaHCO₃ was added and the products extracted into EtOAc (2×). The combined organic extracts were washed with brine, dried over MgSO₄, and concentrated in vacuo. The residue was recrystallized from EtOH to give rac-3-[1-(3-aminophenyl)-1-hydroxyethyl]-1-(4-chlorophenyl)pyridazin-4(1H)-one as a pale brown solid.

LRMS (ESI) calc'd for C18H17ClN3O2 [M+H]⁺: 342. found: 342.

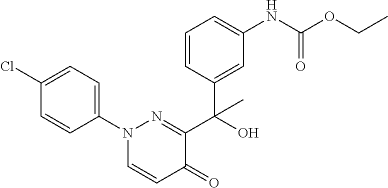

### Step 3. rac-Ethyl(3-{1-[1-(4-chlorophenyl)-4-oxo-1,4-dihydropyridazin-3-yl]-1-hydroxyethyl}phenyl)carbamate

rac-3-[1-(3-Aminophenyl)-1-hydroxyethyl]-1-(4-chlorophenyl)pyridazin-4(1H)-one (87 mg, 0.26 mmol) was taken up in THF (5 mL) and DIPEA (0.049 mL, 0.28 mmol) was added followed by ethyl chloroformate (0.026 mL, 0.27 mmol). The resulting mixture was stirred at r.t. for 45 minutes. Saturated NH₄Cl was added and the aqueous layer was extracted with EtOAc (2×). The combined organic extracts were dried over Na₂SO₄, filtered and concentrated in vacuo to give rac-ethyl(3-{1-[1-(4-chlorophenyl)-4-oxo-1,4-dihydropyridazin-3-yl]-1-hydroxyethyl}phenyl)carbamate as a beige solid.

LRMS (ESI) calc'd for C21H21ClN3O4 [M+H]⁺: 414. found: 414.

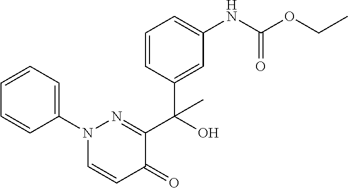

### Step 4. rac-Ethyl {3-[1-hydroxy-1-(4-oxo-1-phenyl-1,4-dihydropyridazin-3-yl)ethyl]phenyl}carbamate

rac-Ethyl(3-{1-[1-(4-chlorophenyl)-4-oxo-1,4-dihydropyridazin-3-yl]-1-hydroxyethyl}phenyl)carbamate (46 mg, 0.11 mmol) and Pd/C (10 wt %, 6.0 mg, 5.6 μmol) were stirred in MeOH (3 mL)/DMA (2 mL) under H₂ (1 atm) at r.t. for 24 hours. Additional Pd/C (10 wt %, 5.9 mg; 5.6 μmol) was added and stirring at r.t. under H₂ continued overnight. The catalyst was removed by filtration through Celite and the solvent removed in vacuo to give rac-ethyl {3-[1-hydroxy-1-(4-oxo-1-phenyl-1,4-dihydropyridazin-3-yl)ethyl]phenyl}carbamate as a yellow gum.

LRMS (ESI) calc'd for C21H22N3O4 [M+H]⁺: 380. found: 380.

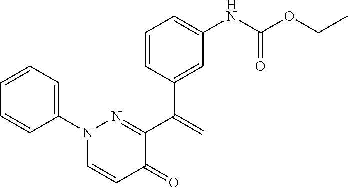

### Step 5. Ethyl{3-[1-(4-oxo-1-phenyl-1,4-dihydropyridazin-3-yl)ethenyl]phenyl}carbamate

rac-Ethyl {3-[1-hydroxy-1-(4-oxo-1-phenyl-1,4-dihydropyridazin-3-yl)ethyl]phenyl}carbamate (42 mg, 0.11 mmol) was stirred in DCM (2 mL)/TFA (2 mL) at r.t. for 4 days. The reaction mixture was warmed to 50° C. overnight. Room temperature was attained and the solvent removed in vacuo. Purification of the residue by flash chromatography (MPLC, 12-100% EtOAc-Hexanes) gave ethyl {3-[1-(4-oxo-1-phenyl-1,4-dihydropyridazin-3-yl)vinyl]phenyl}carbamate as a white solid.

LRMS (ESI) calc'd for C21H20N₃O3 [M+H]⁺: 362. found: 362.

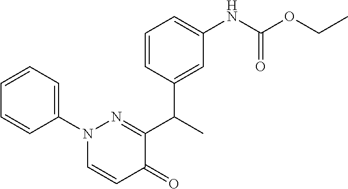

### Step 6. rac-Ethyl{3-[1-(4-oxo-1-phenyl-1,4-dihydropyridazin-3-yl)ethyl]phenyl}carbamate

Ethyl {3-[1-(4-oxo-1-phenyl-1,4-dihydropyridazin-3-yl)vinyl]phenyl}carbamate (24 mg, 0.066 mmol) and Pd/C (10 wt %) (4.0 mg, 3.3 μmol) were stirred in MeOH (0.7 mL)/DMA (0.7 mL) under H₂ (1 atm) at r.t. overnight. The catalyst was removed by filtration through Celite and the solvent removed in vacuo. The residue was purified by flash chromatography (MPLC, 12-100% EtOAc-hexanes) to give rac-ethyl{3-[1-(4-oxo-1-phenyl-1,4-dihydropyridazin-3-yl)ethyl]phenyl}carbamate as a white solid.

LRMS (ESI) calc'd for C21H22N3O3 [M+H]⁺: 364. found: 364.

### Scheme 18

### Examples #652 and 653

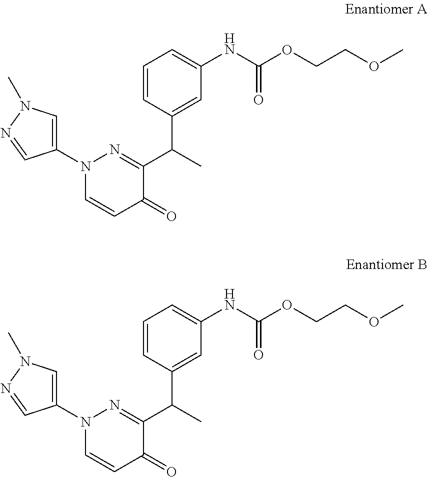

### 2-Methoxyethyl(3-{(1S or R)-1-[1-(1-methyl-1H-pyrazol-4-yl)-4-oxo-1,4-dihydropyridazin-3-yl]ethyl}phenyl)carbamate (Enantiomer A) and 2-methoxyethyl-(3-{(1R or S)-1-[1-(1-methyl-1H-pyrazol-4-yl)-4-oxo-1,4-dihydropyridazin-3-yl]ethyl}phenyl)carbamate (Enantiomer B)

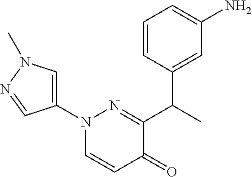

### Step 1. rac-3-[1-(3-Aminophenyl)ethyl]-1-(1-methyl-1H-pyrazol-4-yl)pyridazin-4(1H)-one

rac-1-(1-Methyl-1H-pyrazol-4-yl)-3-[1-(3-nitrophenyl)ethyl]pyridazin-4(1H)-one (Intermediate #144, 70 mg, 0.215 mmol) and [3% Pt+0.6% V]/C (28 mg, 4.301 mol) were stirred in MeOH (2-mL) at room temperature under a balloon of H₂ for 5 hours. The catalyst was removed by filtering through Celite, which was washed with 10% MeOH-DCM to ensure all product eluted through. The filtrate was concentrated in vacuo and the residue purified by flash chromatography (MPLC, 0-15% MeOH-EtOAc) to give rac-3-[1-(3-aminophenyl)ethyl]-1-(1-methyl-1H-pyrazol-4-yl)pyridazin-4(1H)-one as a yellow solid.

LRMS (ESI) calc'd for C16H18N5O [M+H]⁺: 296. Found: 296.

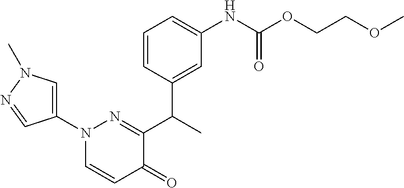

### Step 2. rac-2-Methoxyethyl(3-{1-[1-(1-methyl-1H-pyrazol-4-yl)-4-oxo-1,4-dihydropyridazin-3-yl]ethyl}phenyl)carbamate

rac-3-[1-(3-Aminophenyl)ethyl]-1-(1-methyl-1H-pyrazol-4-yl)pyridazin-4(1H)-one (40 mg, 0.135 mmol) and DIPEA (0.028 mL, 0.163 mmol) were taken up in THF (1.35 mL). 2-Methoxyethyl chloroformate (0.017 mL, 0.149 mmol) was added and the resulting mixture stirred at room temperature for 4 hours. Saturated NaHCO₃ was added and the products extracted into EtOAc (x2). The combined organic extracts were washed with brine, dried over Na₂SO₄, filtered and concentrated in vacuo. The residue was purified by flash chromatography (MPLC, 0-15% MeOH-EtOAc) to give rac-2-methoxyethyl (3-{1-[1-(1-methyl-1H-pyrazol-4-yl)-4-oxo-1,4-dihydropyridazin-3-yl]ethyl}phenyl)carbamate as a pale yellow solid.

LRMS (ESI) calc'd for C20H24N5O4 [M+H]⁺: 398. Found: 398.

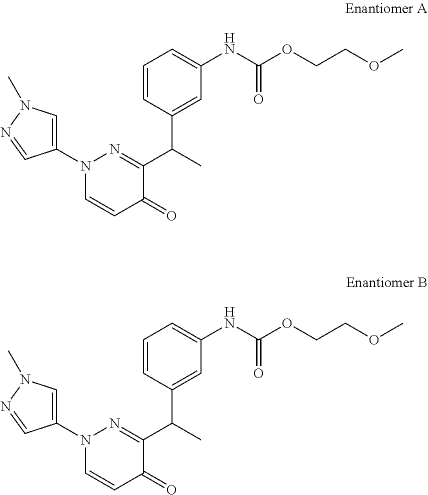

### Step 3. 2-Methoxyethyl(3-{(1S or R)-1-[1-(1-methyl-1H-pyrazol-4-yl)-4-oxo-1,4-dihydropyridazin-3-yl]ethyl}phenyl)carbamate (Enantiomer A) and 2-methoxyethyl (3-{(1R or S)-1-[1-(1-methyl-1H-pyrazol-4-yl)-4-oxo-1,4-dihydropyridazin-3-yl]ethyl}phenyl)carbamate (Enantiomer B)

rac-2-Methoxyethyl(3-{1-[1-(1-methyl-1H-pyrazol-4-yl)-4-oxo-1,4-dihydropyridazin-3-yl]ethyl}phenyl)carbamate (50 mg, 0.126 mmol) was resolved by SFC (Berger Multigram II SFC, column: Chiral Technology OJ-H 2.1×25 cm, 5 μM, mobile phase: 25% 2-propanol/75% CO₂₍ₗ₎, flow rate: 70 mL/min, 9.5 min run time) to give 2-methoxyethyl (3-{(1S or R)-1-[1-(1-methyl-1H-pyrazol-4-yl)-4-oxo-1,4-dihydropyridazin-3-yl]ethyl}phenyl)carbamate (Enantiomer A, Example #652) and 2-methoxyethyl (3-{(1R or S)-1-[1-(1-methyl-1H-pyrazol-4-yl)-4-oxo-1,4-dihydropyridazin-3-yl]ethyl}phenyl)carbamate (Enantiomer B, Example #653).

### Example #652

LRMS (ESI) calc'd for C20H24N₅O4 [M+H]⁺: 398. Found: 398.

### Example #653

LRMS (ESI) calc'd for C20H24N5O4 [M+H]⁺: 398. Found: 398.

### Scheme 19

### Example #654

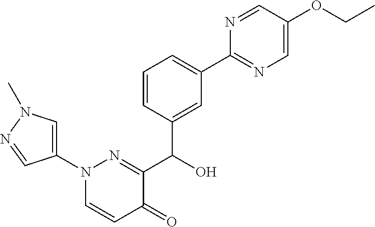

### rac-3-[[3-(5-Ethoxypyrimidin-2-yl)phenyl](hydroxy)methyl]-1-(1-methyl-1H-pyrazol-4-yl)pyridazin-4(1H)-one

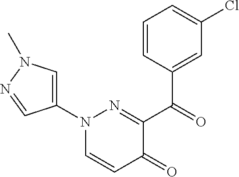

### Step 1. 3-(3-Chlorobenzoyl)-1-(1-methyl-1H-pyrazol-4-yl)pyridazin-4(1H)-one

N-Methoxy-N-methyl-1-(1-methyl-1H-pyrazol-4-yl)-4-oxo-1,4-dihydropyridazine-3-carboxamide (Intermediate #132 Alternative Aniline Synthesis Step 1, 0.535 g, 2.032 mmol) was taken up in THF (2.7 mL) and cooled to 0° C. 3-Chlorophenylmagnesium chloride (0.5 M in THF, 4.07 mL, 2.032 mmol) was added and stirring at 0° C. continued for 3 hours. 2 N HCl (1.02 mL) was added, followed by EtOAc and the yellow solid collected by filtration to give 3-(3-chlorobenzoyl)-1-(1-methyl-1H-pyrazol-4-yl)pyridazin-4(1H)-one.

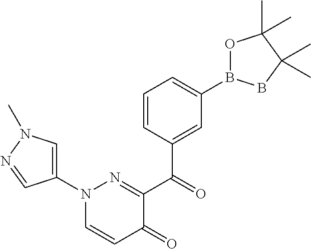

### Step 2. 1-(1-Methyl-1H-pyrazol-4-yl)-3-[3-(4,4,5,5-tetramethyl-1,3,2-dioxaborolan-2-yl)benzoyl]pyridazin-4(1H)-one

3-(3-Chlorobenzoyl)-1-(1-methyl-1H-pyrazol-4-yl)pyridazin-4(1H)-one (0.75 g, 2.383 mmol), B₂Pin₂ (0.908 g, 3.57 mmol), Pd₂(dba)₃ (0.044 g, 0.048 mmol), X-Phos (0.091 g, 0.191 mmol) and KOAc (0.702 g, 7.15 mmol) were taken up in 1,4-dioxane (24 mL) in a 100 mL round bottom flask. The flask was evacuated and back-filled with N₂ (x3) before stirring at 100° C. for 2 hours. Room temperature was attained, saturated NH₄Cl was added and the products extracted into EtOAc (x2). The combined organic extracts were washed with saturated NaHCO₃ and brine, dried over Na₂SO₄, filtered through Celite and concentrated in vacuo. The residue was triturated in hexanes to give 1-(1-methyl-1H-pyrazol-4-yl)-3-[3-(4,4,5,5-tetramethyl-1,3,2-dioxaborolan-2-yl)benzoyl]pyridazin-4(1H)-one as a pale yellow solid.

LRMS (ESI) calc'd for C21H24BN4O4 [M+H]⁺: 407. Found: 407.

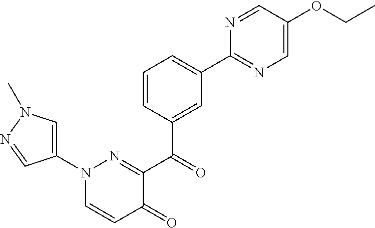

### Step 3. 3-[3-(5-Ethoxypyrimidin-2-yl)benzoyl]-1-(1-methyl-1H-pyrazol-4-yl)pyridazin-4(1H)-one

2-Chloro-5-ethoxypyrimidine (Intermediate #86 Step 3, 59 mg, 0.369 mmol), 1-(1-methyl-1H-pyrazol-4-yl)-3-[3-(4,4,5,5-tetramethyl-1,3,2-dioxaborolan-2-yl)benzoyl]pyridazin-4(1H)-one (100 mg, 0.246 mmol), PdCl₂(dppf).DCM adduct (0.020 g, 0.025 mmol) and 2 N Na₂CO₃ (0.246 mL, 0.492 mmol) were taken up in 1,4-dioxane (1.25 mL) in a 20 mL microwave vial. The vial was evacuated and back-filled with N₂ (x3) before stirring at 100° C. for 50 minutes. Room temperature was attained and saturated NH₄Cl and EtOAc were added. The biphasic suspension was filtered and the solid product taken up in hot DMF. The hot solution was filtered through Celite and washed with DMF followed by MeOH. The filtrate was concentrated in vacuo and the residue triturated in MeOH to give 3-[3-(5-ethoxypyrimidin-2-yl)benzoyl]-1-(1-methyl-1H-pyrazol-4-yl)pyridazin-4(1H)-one as a beige solid.

LRMS (ESI) calc'd for C21H19N6O3 [M+H]⁺: 403. Found: 403.

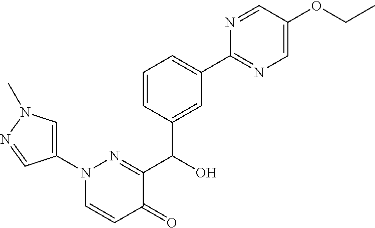

### Step 4. rac-3-[[3-(5-Ethoxypyrimidin-2-yl)phenyl](hydroxy)methyl]-1-(1-methyl-1H-pyrazol-4-yl)pyridazin-4(1H)-one

rac-3-[3-(5-Ethoxypyrimidin-2-yl)benzoyl]-1-(1-methyl-1H-pyrazol-4-yl)pyridazin-4(1H)-one (145 mg, 0.360 mmol) and NaBH₄ (15 mg, 0.396 mmol) were stirred in MeOH (3.6 mL) at room temperature for 5 hours. Additional NaBH₄ (6.8 mg, 0.180 mmol) was added and the resulting mixture stirred at room temperature for 18 hours. Saturated NH₄Cl was added and the products extracted into EtOAc (x3). The combined organic extracts were washed with brine, dried over Na₂SO₄ and concentrated in vacuo to give rac-3-[[3-(5-ethoxypyrimidin-2-yl)phenyl](hydroxy)methyl]-1-(1-methyl-1H-pyrazol-4-yl)pyridazin-4(1H)-one as a pale yellow solid.

LRMS (ESI) calc'd for C21H21N6O3 [M+H]⁺: 405. Found: 405.

### Scheme 19

### Examples #655 and 656

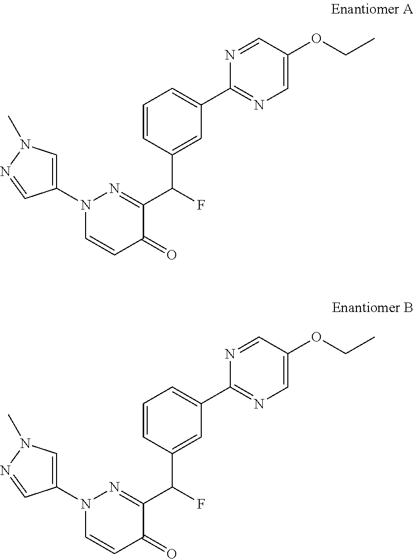

### 3-[(S or R)-[3-(5-Ethoxypyrimidin-2-yl)phenyl](fluoro)methyl]-1-(1-methyl-1H-pyrazol-4-yl)pyridazin-4(1H)-one (Enantiomer A) and 3-[(R or S)-[3-(5-ethoxypyrimidin-2-yl)phenyl](fluoro)methyl]-1-(1-methyl-1H-pyrazol-4-yl)pyridazin-4(1H)-one (Enantiomer B)

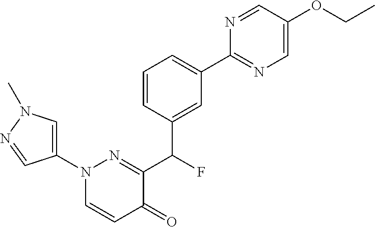

### Step 1. rac-3-[[3-(5-Ethoxypyrimidin-2-yl)phenyl](fluoro)methyl]-1-(1-methyl-1H-pyrazol-4-yl)pyridazin-4(1H)-one

rac-3-[[3-(5-Ethoxypyrimidin-2-yl)phenyl](hydroxy)methyl]-1-(1-methyl-1H-pyrazol-4-yl)pyridazin-4(1H)-one (Example #654, 100 mg, 0.247 mmol) and bis(2-methoxyethyl)aminosulfurtrifluoride (0.055 mL, 0.297 mmol) were stirred in DCM (2.5 mL) at room temperature for 3 hours. Saturated NaHCO₃ was added and the products extracted into EtOAc (x2). The combined organic extracts were washed with brine, dried over Na₂SO₄ and concentrated in vacuo. Purification of the residue by flash chromatography (MPLC, 0-10% MeOH-EtOAc) gave rac-3-[[3-(5-ethoxypyrimidin-2-yl)phenyl](fluoro)methyl]-1-(1-methyl-1H-pyrazol-4-yl)pyridazin-4(1H)-one as a pale yellow solid.

LRMS (ESI) calc'd for C21H20FN6O2 [M+H]⁺: 407. Found: 407.

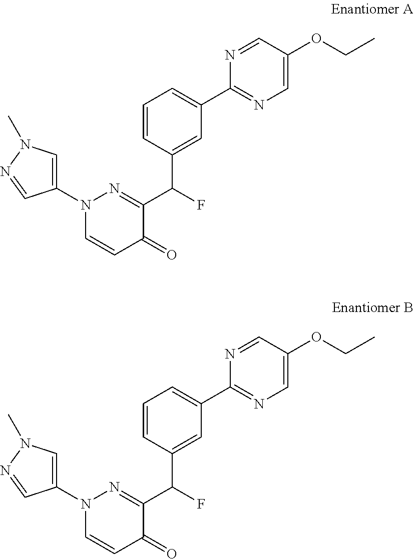

### Step 2. 3-[(S or R)-[3-(5-Ethoxypyrimidin-2-yl)phenyl](fluoro)methyl]-1-(1-methyl-1H-pyrazol-4-yl)pyridazin-4(1H)-one (Enantiomer A) and 3-[(R or S)-[3-(5-ethoxypyrimidin-2-yl)phenyl](fluoro)methyl]-1-(1-methyl-1H-pyrazol-4-yl)pyridazin-4(1H)-one (Enantiomer B)

rac-3-[[3-(5-Ethoxypyrimidin-2-yl)phenyl](fluoro)methyl]-1-(1-methyl-1H-pyrazol-4-yl)pyridazin-4(1H)-one (88 mg, 0.217 mmol) was resolved by SFC (Berger Multigram II SFC, column: Chiral Technology AS-H 2.1×25 cm, 5 μM, mobile phase: 40% methanol/60% CO₂₍ₗ₎, flow rate: 60 mL/min, 8 min run time) to give 3-[(S or R)-[3-(5-ethoxypyrimidin-2-yl)phenyl](fluoro)methyl]-1-(1-methyl-1H-pyrazol-4-yl)pyridazin-4(1H)-one (Enantiomer A, Example #655) and 3-[(R or S)-[3-(5-ethoxypyrimidin-2-yl)phenyl](fluoro)methyl]-1-(1-methyl-1H-pyrazol-4-yl)pyridazin-4(1H)-one (Enantiomer B, Example #656).

### Example #655

LRMS (ESI) calc'd for C21H20FN6O2 [M+H]⁺: 407. Found: 407.

### Example #656

LRMS (ESI) calc'd for C21H20FN6O2 [M+H]⁺: 407. Found: 407.

### Scheme 19

### Example #657

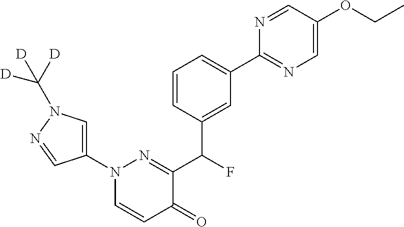

### rac-3-[[3-(5-Ethoxypyrimidin-2-yl)phenyl](fluoro)methyl]-1-(1-methyl-1H-pyrazol-4-yl)pyridazin-4(1H)-one-d3

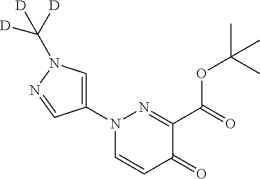

### Step 1. tert-Butyl 1-(1-methyl-1H-pyrazol-4-yl)-4-oxo-1,4-dihydropyridazine-3-carboxylate-d3

tert-Butyl 4-oxo-1-(1-pyrazol-4-yl)-1,4-dihydropyridazine-3-carboxylate (Intermediate #27 Step 2, 509 mg, 1.941 mmol), Cs₂CO₃(949 mg, 2.91 mmol) and iodomethane-d3 (0.181 mL, 2.91 mmol) were stirred in THF (5 mL)/DMF (5 mL) at 80° C. for 18 hours. The solvent was removed in vacuo while loading onto silica and the residue purified by flash chromatography (MPLC, 0-10% MeOH-EtOAc) to give tert-butyl 1-(1-methyl-1H-pyrazol-4-yl)-4-oxo-1,4-dihydropyridazine-3-carboxylate-d3 as a pale yellow solid.

LRMS (ESI) calc'd for C9H₆D3N₄O3⁻ [M+H]⁺: 224. Found: 224 (carboxylic acid).

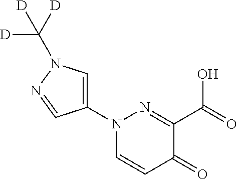

### Step 2. 1-(1-Methyl-1H-pyrazol-4-yl)-4-oxo-1,4-dihydropyridazine-3-carboxylic acid-d3

1-(1-Methyl-1H-pyrazol-4-yl)-4-oxo-1,4-dihydropyridazine-3-carboxylic acid-d3 was prepared from tert-butyl 1-(1-methyl-1H-pyrazol-4-yl)-4-oxo-1,4-dihydropyridazine-3-carboxylate-d3 according to the procedure described for 1-(1-methyl-1H-pyrazol-4-yl)-4-oxo-1,4-dihydropyridazine-3-carboxylic acid (Intermediate #1 Step 3).

LRMS (ESI) calc'd for C9H₆D3N4O3 [M+H]⁺: 224. Found: 224.

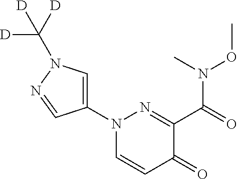

### Step 3. N-Methoxy-N-methyl-1-(1-methyl-1H-pyrazol-4-yl)-4-oxo-1,4-dihydropyridazine-3-carboxamide-d3

N-Methoxy-N-methyl-1-(1-methyl-1H-pyrazol-4-yl)-4-oxo-1,4-dihydropyridazine-3-carboxamide-d3 was prepared from 1-(1-methyl-1H-pyrazol-4-yl)-4-oxo-1,4-dihydropyridazine-3-carboxylic acid-d3 according to the procedure described for N-methoxy-N-methyl-1-(1-methyl-1H-pyrazol-4-yl)-4-oxo-1,4-dihydropyridazine-3-carboxamide (Intermediate #132 Alternative Aniline Synthesis Step 1).

LRMS (ESI) calc'd for C11H11D3N5O3 [M+H]⁺: 267. Found: 267.

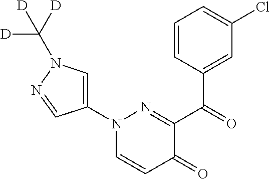

### Step 4. 3-(3-Chlorobenzoyl)-1-(1-methyl-1H-pyrazol-4-yl)pyridazin-4(1H)-one-d3

3-(3-Chlorobenzoyl)-1-(1-methyl-1H-pyrazol-4-yl)pyridazin-4(1H)-one-d3 was prepared from N-methoxy-N-methyl-1-(1-methyl-1H-pyrazol-4-yl)-4-oxo-1,4-dihydropyridazine-3-carboxamide-d3 according to the procedure described for 3-(3-chlorobenzoyl)-1-(1-methyl-1H-pyrazol-4-yl)pyridazin-4(1H)-one (Example #654 Step 1).

LRMS (ESI) calc'd for C15H9D3ClN4O2 [M+H]⁺: 318. Found: 318.

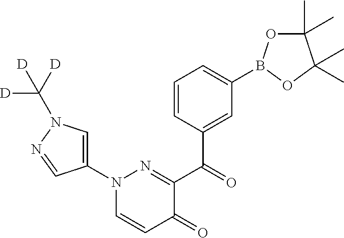

### Step 5. 1-(1-Methyl-1H-pyrazol-4-yl)-3-[3-(4,4,5,5-tetramethyl-1,3,2-dioxaborolan-2-yl)benzoyl]pyridazin-4(1H)-one-d3

1-(1-Methyl-1H-pyrazol-4-yl)-3-[3-(4,4,5,5-tetramethyl-1,3,2-dioxaborolan-2-yl)benzoyl]pyridazin-4(1H)-one-d3 was prepared from 3-(3-chlorobenzoyl)-1-(1-methyl-1H-pyrazol-4-yl)pyridazin-4(1H)-one-d3 according to the procedure described for 1-(1-methyl-1H-pyrazol-4-yl)-3-[3-(4,4,5,5-tetramethyl-1,3,2-dioxaborolan-2-yl)benzoyl]pyridazin-4(1H)-one-(Example #654 Step 2).

LRMS (ESI) calc'd for C21H21D3BN4O4 [M+H]⁺: 410. Found: 410.

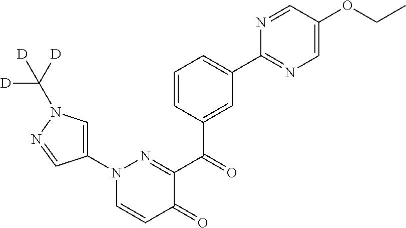

### Step 6. 3-[3-(5-Ethoxypyrimidin-2-yl)benzoyl]-1-(1-methyl-1H-pyrazol-4-yl)pyridazin-4(1H)-one-d3

3-[3-(5-Ethoxypyrimidin-2-yl)benzoyl]-1-(1-methyl-1H-pyrazol-4-yl)pyridazin-4(1H)-one-d3 was prepared from 1-(1-methyl-1H-pyrazol-4-yl)-3-[3-(4,4,5,5-tetramethyl-1,3,2-dioxaborolan-2-yl)benzoyl]pyridazin-4(1H)-one-d3 and 2-chloro-5-ethoxypyrimidine (Intermediate #86 Step 3) according to the procedure described for 3-[3-(5-ethoxypyrimidin-2-yl)benzoyl]-1-(1-methyl-1H-pyrazol-4-yl)pyridazin-4(1H)-one (Example #654 Step 3).

LRMS-(ESI)-calc'd for C21H16D3N6O3 [M+H]⁺: 406. Found: 406.

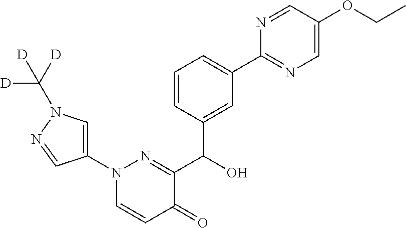

### Step 7. rac-3-[[3-(5-Ethoxypyrimidin-2-yl-phenyl](hydroxy)methyl]-1-(1-methyl-1H-pyrazol-4-yl)pyridazin-4(1H)-one-d3

rac-3-[[3-(5-Ethoxypyrimidin-2-yl)phenyl](hydroxy)methyl]-1-(1-methyl-1H-pyrazol-4-yl)pyridazin-4(1H)-one-d3 was prepared from 3-[3-(5-ethoxypyrimidin-2-yl)benzoyl]-1-(1-methyl-1H-pyrazol-4-yl)pyridazin-4(1H)-one-d3 according to the procedure described for rac-3-[[3-(5-ethoxypyrimidin-2-yl)phenyl](hydroxy)methyl]-1-(1-methyl-1H-pyrazol-4-yl)pyridazin-4(1H)-one (Example #654 Step 4).

LRMS (ESI) calc'd for C21H18D3N6O3 [M+H]⁺: 408. Found: 408.

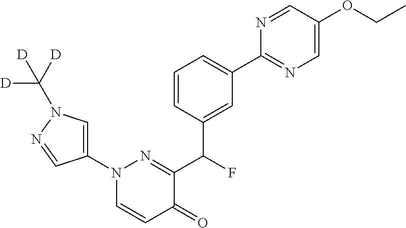

### Step 8. rac-3-[[3-(5-Ethoxypyrimidin-2-yl)phenyl](fluoro)methyl]-1-(1-methyl-1H-pyrazol-4-yl)pyridazin-4(1H)-one-d3

rac-3-[[3-(5-Ethoxypyrimidin-2-yl)phenyl](fluoro)methyl]-1-(1-methyl-1H-pyrazol-4-yl)pyridazin-4(1H)-one-d3 was prepared from 3-[[3-(5-ethoxypyrimidin-2-yl)phenyl](hydroxy)methyl]-1-(1-methyl-1H-pyrazol-4-yl)pyridazin-4(1H)-one-d3 according to the procedure described for rac-3-[[3-(5-ethoxypyrimidin-2-yl)phenyl](fluoro)methyl]-1-(1-methyl-1H-pyrazol-4-yl)pyridazin-4(1H)-one (Examples #655-656 Step 1).

LRMS (ESI) calc'd for C21H17D3FN6O2 [M+H]⁺: 410. Found: 410.

### Scheme 19

### Example #658

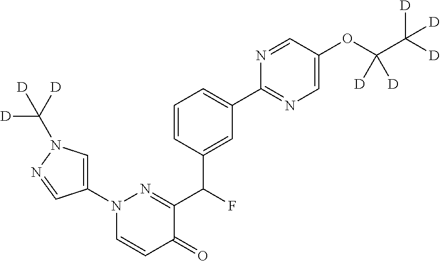

### rac-3-[[3-(5-Ethoxypyrimidin-2-yl)phenyl](fluoro)methyl]-1-(1-methyl-1H-pyrazol-4-yl)pyridazin-4(1H)-one-d8

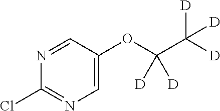

### Step 1. 2-Chloro-5-ethoxypyrimidine-d5

2-Chloro-5-ethoxypyrimidine-d5 was prepared from 2-chloropyrimidin-5-ol (Intermediate #86 Step 2) and iodoethane-d5 according to the procedure described for 2-chloro-5-ethoxypyrimidine (Intermediate #86 Step 3).

LRMS (ESI) calc'd for C6H₃D5ClN2O [M+H]⁺: 164. Found: 164.

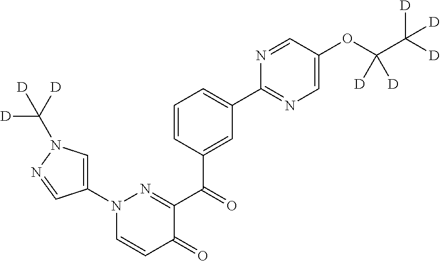

### Step 2. 3-[3-(5-Ethoxypyrimidin-2-yl)benzoyl]-(1-methyl-1H-pyrazol-4-yl)pyridazin-4(1H)-one-d8

3-[3-(5-Ethoxypyrimidin-2-yl)benzoyl]-1-(1-methyl-1H-pyrazol-4-yl)pyridazin-4(1H1)-one-d8 was prepared from 1-(1-methyl-1H-pyrazol-4-yl)-3-[3-(4,4,5,5-tetramethyl-1,3,2-dioxaborolan-2-yl)benzoyl]pyridazin-4(1H)-one-d3 (Example #657 Step 5) and 2-chloro-5-ethoxypyrimidine-d5 according to the procedure described for 3-[3-(5-ethoxypyrimidin-2-yl)benzoyl]-1-(1-methyl-1H-pyrazol-4-yl)pyridazin-4(1H)-one (Example #657 Step 3).

LRMS (ESI) calc'd for C21H11D8N6O3 [M+H]⁺: 411. Found: 411.

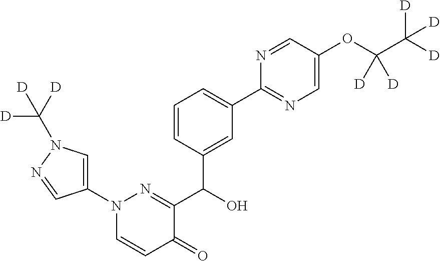

### Step 3. rac-3-[[3-(5-Ethoxypyrimidin-2-yl)phenyl](hydroxy)methyl]-1-(1-methyl-1H-pyrazol-4-yl)pyridazin-4(1H)-one-d8

rac-3-[[3-(5-Ethoxypyrimidin-2-yl)phenyl](hydroxy)methyl]-1-(1-methyl-1H-5-pyrazol-4-yl)pyridazin-4(1H)-one-d8 was prepared from 3-[3-(5-ethoxypyrimidin-2-yl)benzoyl]-1-(1-methyl-1H-pyrazol-4-yl)pyridazin-4(1H)-one-d8 according to the procedure described for rac-3-[[3-(5-ethoxypyrimidin-2-yl)phenyl](hydroxy)methyl]-1-(1-methyl-1H-pyrazol-4-yl)pyridazin-4(1H)-one (Example #654 Step 4).

LRMS (ESI) calc'd for C21H13D8N6O3 [M+H]⁺: 413. Found: 413.

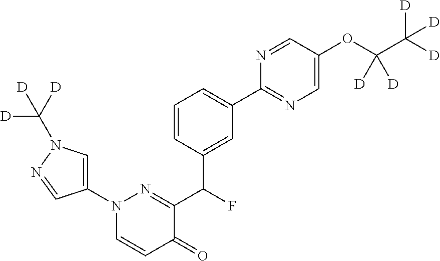

### Step 4. rac-3-[[3-(5-Ethoxypyrimidin-2-yl)phenyl](fluoro)methyl]-1-(1-methyl-1H-pyrazol-4-yl)pyridazin-4(1H)-one-d8

rac-3-[[3-(5-Ethoxypyrimidin-2-yl)phenyl](fluoro)methyl]-1-(1-methyl-1H-pyrazol-4-yl)pyridazin-4(1H)-one-d8 was prepared from 3-[[3-(5-ethoxypyrimidin-2-yl)phenyl](hydroxy)methyl]-1-(1-methyl-1H-pyrazol-4-yl)pyridazin-4(1H)-one-d8 according to the procedure described for rac-3-[[3-(5-ethoxypyrimidin-2-yl)phenyl](fluoro)methyl]-1-(1-methyl-1H-pyrazol-4-yl)pyridazin-4(1H)-one (Examples #655-656 Step 1).

LRMS (ESI) calc'd for C21H12D8FN6O2 [M+H]⁺: 415. Found: 415.

### Scheme 19

### Example #659

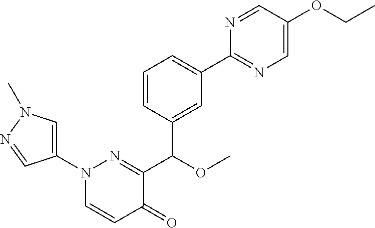

### rac-3-[[3-(5-Ethoxypyrimidin-2-yl)phenyl](methoxy)methyl]-1-(1-methyl-1H-pyrazol-4-yl)pyridazin-4(1H)-one

Step 1. rac-3-[[3-(5-Ethoxypyrimidin-2-yl)phenyl](methoxy)methyl]-1-(1-methyl-1H-pyrazol-4-yl)pyridazin-4(1H)-one

rac-3-[[3-(5-Ethoxypyrimidin-2-yl)phenyl](hydroxy)methyl]-1-(1-methyl-1H-pyrazol-4-yl)pyridazin-4(1H)-one (Example #654, 46 mg, 0.114 mmol) and iodomethane (0.021 mL, 0.341 mmol) were taken up in DMF (1.1 mL). NaH (60 wt %, 14 mg, 0.34 mmol) was added and the resulting mixture stirred at room temperature for 3 hours. Saturated NH₄Cl was added and the products extracted into EtOAc (x2). The combined organic extracts were washed with brine, dried over Na₂SO₄ and concentrated in vacuo. Purification of the residue by flash chromatography (MPLC, 0-10% MeOH-EtOAc) gave rac-3-[[3-(5-ethoxypyrimidin-2-yl)phenyl](methoxy)methyl]-1-(1-methyl-1H-pyrazol-4-yl)pyridazin-4(1H)-one as a yellow solid.

LRMS (ESI) calc'd for C22H23N₆O3 [M+H]⁺: 419. Found: 419.

### Scheme 20

### Example #660

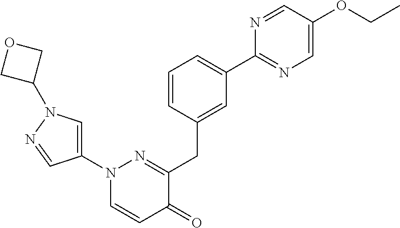

### 3-[3-(5-Ethoxypyrimidin-2-yl)benzyl]-1-[1-(oxetan-3-yl)-1H-pyrazol-4-yl]pyridazin-4(1H)-one

### Step 1. 3-[3-(5-Ethoxypyrimidin-2-yl)benzyl]-1-[1-(oxetan-3-yl-1H-pyrazol-4-yl]pyridazin-4(1H)-one

3-[3-(5-Ethoxypyrimidin-2-yl)benzyl]-1-(1H-pyrazol-4-yl)pyridazin-4(1H)-one (Example #195, 30 mg, 0.080 mmol), 3-iodooxetane (22 mg, 0.120 mmol), and Cs₂CO₃ (78 mg, 0.240 mmol) were combined in a 2 mL microwave vial. The vial was evacuated and back-filled with N₂ before adding DMF (2 mL). The reaction mixture was heated to 150° C. for 30-minutes under microwave irradiation-(Biotage, Initiator). The solvent was removed by centrifugation and the dry residue suspended in MeOH/DCM before filtering through cotton wool. The filtrate was concentrated in vacuo and the residue purified by mass-triggered reverse-phase preparative HPLC. Product fractions were concentrated by centrifugation and the solid taken up in 4 mL of MeOH. The solution was eluted through a PL-HCO₃ cartridge (Stratospheres™, 0.9 mmol) and the filtrate concentrated in vacuo to give 3-[3-(5-ethoxypyrimidin-2-yl)benzyl]-1-[1-(oxetan-3-yl)-1H-pyrazol-4-yl]pyridazin-4(1H)-one.

LRMS (ESI) calc'd for C23H23N6O3 [M+H]⁺: 431. Found 431.

The following examples were prepared according to Scheme 20 following a similar procedure described for Example #660, which can be achieved by those of ordinary skill in the art of organic synthesis.

| | | | Exact |
|---|---|---|---|
| | | IUPAC | Mass |
| Example | Structure | Name | [M + H]⁺ |
| 661 | 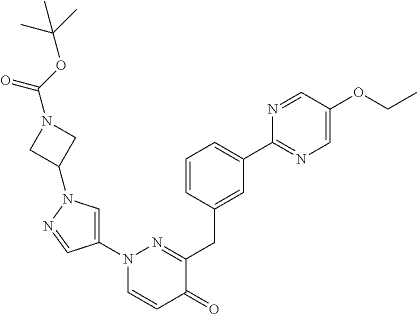 | tert-butyl 3- (4-{3-[3-(5- ethoxypyrimidin-2- yl)benzyl]-4- oxopyridazin- 1(4H)-yl}-1H- pyrazol-1- yl)acetidine-1- carboxylate | Calc'd 530, found 530 |
| 662 | 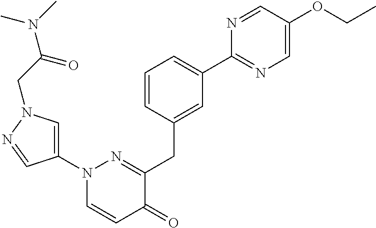 | 2-(4-{3-[3-(5- ethoxypyrimidin- 2- yl)benzyl]-4- oxopyridazin- 1(4H)-yl}-1H- pyrazol-1-yl)- N,N- dimethylacetamide | Calc'd 460, found 460 |

The following intermediate was prepared according to Scheme 20 following a similar procedure described for Example #660, which can be achieved by those of ordinary skill in the art of organic synthesis.

| | | | Exact |
|---|---|---|---|
| | | IUPAC | Mass |
| Intermediate | Structure | Name | [M + H]⁺ |
| 175 | 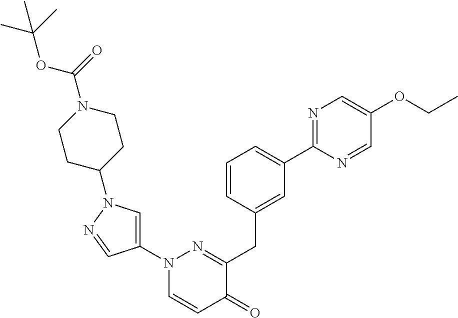 | tert-butyl 4- {4-[3-[3-(5- ethoxypyrimidin- 2- yl)benzyl]-4- oxopyridazin- 1(4H)-yl]-1H- pyrazol-1- yl}piperidine- 1-carboxylate | Calc'd 558, found 558 |

### Scheme 20

### Example #663

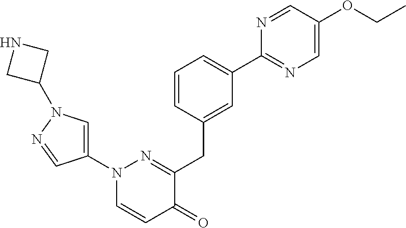

### 1-(1-Azetidin-3-yl-1H-pyrazol-4-yl)-3-[3-(5-ethoxypyrimidin-2-yl)benzyl]pyridazin-4(1H)-one

### Step 1. 1-[1-(Azetidin-3-yl)-1H-pyrazol-4-yl]-3-[3-(5-ethoxypyrimidin-2-yl)benzyl]pyridazin-4(1H)-one

tert-Butyl 3-(4-{3-[3-(5-ethoxypyrimidin-2-yl)benzyl]-4-oxopyridazin-(4H)-yl}-1H-pyrazol-1-yl)azetidine-1-carboxylate (Example #661, 111 mg, 0.210 mmol) was dissolved in MeOH (2 mL) and 4N HCl in 1,4-dioxane (0.1 mL) was added. The mixture was allowed to stir at 80° C. overnight. The mixture was concentrated in vacuo, resuspended in MeOH and passed through 2 PL-HCO₃ cartridges (Stratospheres™, 0.9 mmol). The filtrate was concentrated in vacuo to give 1-[1-(azetidin-3-yl)-1H-pyrazol-4-yl]-3-[3-(5-ethoxypyrimidin-2-yl)benzyl]pyridazin-4(1H)-one.

LRMS (ESI) calc'd for C231H24N7O2 [M+H]⁺: 430. Found 430

The following example was prepared from Intermediate #175 according to Scheme 20 following a similar procedure described for Example #663, which can be achieved by those of ordinary skill in the art of organic synthesis.

| | | | Exact |
|---|---|---|---|
| | | IUPAC | Mass |
| Example | Structure | Name | [M + H]⁺ |
| 664 | 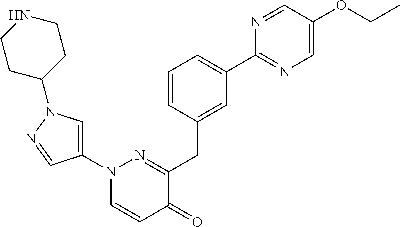 | 3-[3-(5- ethoxypyrimidin- 2- yl)benzyl]-1- (1-piperidin- 4-yl-1H- pyrazol-4- yl)pyridazin- 4(1H)-one | Calc'd 458, found 458 |

### Scheme 20

### Example #665

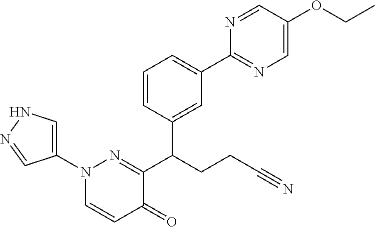

rac-4-[3-(5-Ethoxypyrimidin-2-yl)phenyl]-4-[4-oxo-1-(1H-pyrazol-4-yl)-1,4-dihydropyridazin-3-yl]butanenitrile

### Step 1. rac-4-[3-(5-Ethoxypyrimidin-2-yl)phenyl]-4-[4-oxo-1-(1H-pyrazol-4-yl)-1,4-dihydropyridazin-3-yl]butanenitrile

3-[3-(5-Ethoxypyrimidin-2-yl)benzyl]-1-(1H-pyrazol-4-yl)pyridazin-4(1H)-one (Example #195, 30 mg, 0.080 mmol), 3-bromopropionitrile (10 μL, 0.120 mmol) and Cs₂CO₃ (78 mg, 0.240 mmol) were combined in a 2 mL microwave vial. The vial was evacuated and back-filled with N₂ before adding DMF (2 mL). The reaction mixture was heated to 150° C. for 30 minutes under microwave irradiation (Biotage, Initiator). The solvent was removed by centrifugation and the dry residue suspended in MeOH/DCM and filtered through cotton wool. The filtrate was concentrated in vacuo and the residue purified by mass-triggered reverse-phase preparative HPLC to give rac-4-[3-(5-ethoxypyrimidin-2-yl)phenyl]-4-[4-oxo-1-(1H-pyrazol-4-yl)-1,4-dihydropyridazin-3-yl]butanenitrile.

LRMS (ESI) calc'd for C23H22N7O2 [M+H]⁺: 428. Found 428.

### Scheme 20

### Example #666

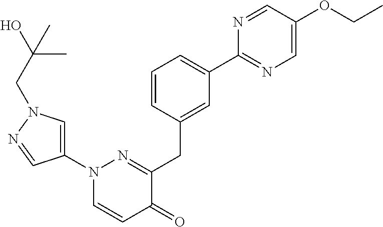

### 3-[3-(5-Ethoxypyrimidin-2-yl)benzyl]-1-[1-(2-hydroxy-2-methylpropyl)-1H-pyrazol-4-yl]pyridazin-4(1H)-one

### Step 1. 3-[3-(5-Ethoxypyrimidin-2-yl)benzyl]-1-[1-(2-hydroxy-2-methylpropyl-H—pyrazol-4-yl]pyridazin-4(1H)-one

3-[3-(5-Ethoxypyrimidin-2-yl)benzyl]-1-(1H-pyrazol-4-yl)pyridazin-4(1H)-one, (Example #195, 64 mg, 0.171 mmol), isobutylene oxide (0.017 mL, 0.188 mmol), and Cs₂CO₃ (167 mg, 0.513 mmol) were combined in a 2 mL microwave vial. The vial was evacuated and back-filled with N₂ before adding DMF (2 mL). The reaction mixture was heated to 150° C. for minutes under microwave irradiation (Biotage, Initiator). Saturated NH₄Cl was added and the products extracted into MeOH/DCM (3×). The combined organic extracts were washed with brine, dried over Na₂SO₄, filtered and concentrated in vacuo. Purification of the residue by flash chromatography (MPLC, 0-15% MeOH-DCM) gave 3-[3-(5-ethoxypyrimidin-2-yl)benzyl]-1-[1-(2-hydroxy-2-methylpropyl)-1H-pyrazol-4-yl]pyridazin-4(1H)-one.

LRMS (ESI) calc'd for C24H27N₆O3 [M+H]⁺: 447. Found 447.

### Scheme 20

### Example #667

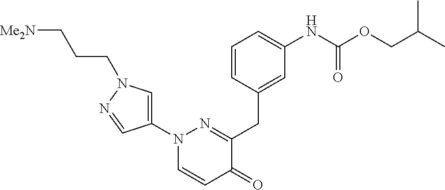

### 2-Methylpropyl {3-[(1-{1-[3-(dimethylamino)propyl]-1H-pyrazol-4-yl}-4-exo-1,4-dihydropyridazin-3-yl)methyl]phenyl}carbamate

### Step 1. 2-Methylpropyl(3-{4-oxo-1-(1H-pyrazol-4-yl)-1,4-dihydropyridazin-3-yl)methyl}phenyl)carbamate

To a solution of isobutyl (3-{[4-oxo-1-(1H-pyrazol-4-yl)-1,4-dihydropyridazin-3-yl]methyl}phenyl)carbamate (Example #197, 10 mg, 0.027 mmol) in THF (272 μL) was added 3-dimethylaminopropanol (3.0 mg, 0.033 mmol) and triphenylphosphine (9 mg, 0.035 mmol). To this solution was added diisopropyl azodicarboxylate (6.0 μL, 0.033 mmol) over 1 minute. Upon complete conversion the reaction mixture was concentrated and purified by reverse phase preparative HPLC (10-100% MeCN—H₂O, 0.05% TFA). After removal of the solvent in vacuo the free base was liberated by dissolving the residue in DCM/MeOH (4:1), filtering through PL-HCO₃ cartridges (Stratospheres™, 0.9 mmol) and concentrating in vacuo to afford 2-methylpropyl (3-{[4-oxo-1-(1H-pyrazol-4-yl)-1,4-dihydropyridazin-3-yl]methyl}phenyl)carbamate.

LRMS (ESI) calc'd for C24H33N6O3 [M+H]⁺: 453. Found: 453.

### Scheme 21

### Example #668

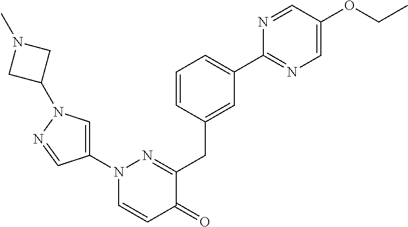

### 3-[3-(5-Ethoxypyrimidin-2-yl)benzyl]-1-[1-(1-methylazetidin-3-yl)-1H-pyrazol-4-yl]pyridazin-4(1H)-one

### Step 1. 3-[3-(5-Ethoxy pyrimidin-2-yl)benzyl]-1-[1-(1-methylazetidin-3-yl)-1H-pyrazol-4-yl]pyridazin-4(1H)-one

1-[1-(Azetidin-3-yl)-1H-pyrazol-4-yl]-3-[3-(5-ethoxypyrimidin-2-yl)benzyl]pyridazin-4(1H)-one (Example #663, 52 mg, 0.121 mmol) and Cs₂CO₃ (43 mg, 0.133 mmol) were combined in DMF (2 mL) before adding iodomethane (8.3 μL, 0.1.3 mmol). The mixture was allowed to stir at ambient temperature for 1 hour. Saturated NH₄Cl was added and the products extracted into DCM/MeOH (x4). The combined organic extracts were washed with brine, dried over Na₂SO₄, filtered and concentrated in vacuo. Purification of the residue by flash chromatography (MPLC, 0-15% MeOH-DCM) gave 3-[3-(5-ethoxypyrimidin-2-yl)benzyl]-1-[1-(1-methylazetidin-3-yl)-1H-pyrazol-4-yl]pyridazin-4(1H)-one.

LRMS (ESI) calc'd for C24H26N7O2 [M+H]⁺: 444. Found 444.

### Scheme 22

### Example #669

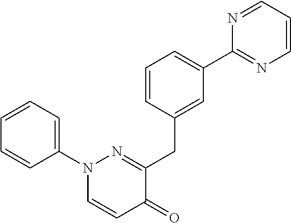

### 1-Phenyl-3-[3-(pyrimidin-2-yl)benzyl]pyridazin-4(1H)-one

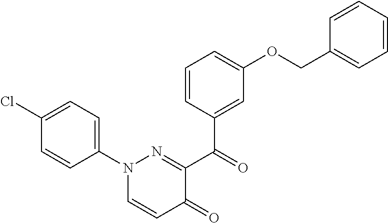

### Step 1. 3-[3-(Benzyloxy)benzoyl]-1-(4-chlorophenyl)pyridazin-4(1H-one

A solution of 3-(benzyloxy)phenylmagnesium bromide (3M, 7.0 mL, 7.0 mmol) in THF was added dropwise to 1-(4-chlorophenyl)-N-methoxy-N-methyl-4-oxo-1,4-dihydropyridazine-3-carboxamide (Example #640 Step 1, 0.83 g, 2.8 mmol) in THF (28 mL) at −78° C., and the mixture was allowed to stir at −78° C. for 45 minutes. 2N HCl and water were added dropwise and the mixture was allowed to warm to r.t. EtOAc and Na₂CO₃ were added. The aqueous layer was extracted with EtOAc (3×) and the combined organic layers were washed with brine, dried over Na₂SO₄, and concentrated in vacuo. Trituration with diethyl ether gave 3-{[4-(benzyloxy)phenyl]carbonyl}-1-(4-chlorophenyl)pyridazin-4(1H)-one as a brown/orange solid.

LRMS (ESI) calc'd for C24H17ClN2O3 [M+H]⁺: 417. Found: 417.

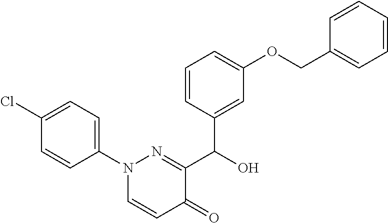

### Step 2. rac-3-{[3-(Benzyloxy)phenyl](hydroxy)methyl}-1-(4-chlorophenyl)pyridazin-4(1H)-one

Sodium borohydride (61 mg, 1.6 mmol) was added portionwise to 3-{[3-(benzyloxy)phenyl]carbonyl}-1-(4-chlorophenyl)pyridazin-4(1H)-one (497 mg, 1.46 mmol) in MeOH (1.5 mL). The reaction mixture was allowed to stir at r.t. for 1 hour, and water and EtOAc were added. The aqueous layer was extracted with EtOAc (2×) and the combined organic layers were washed with brine, dried over Na₂SO₄, and concentrated in vacuo. Trituration with diethyl ether-gave rac-3-{[3-(benzyloxy)phenyl](hydroxy)methyl}-1-(4-chlorophenyl)pyridazin-4(1H)-one as a pale yellow solid.

LRMS (ESI) calc'd for C24H20ClN2O3 [M+H]⁺: 419. Found: 419.

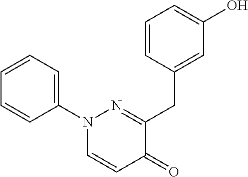

### Step 3. 3-(3-Hydroxybenzyl)-1-phenylpyridazin-4(1H)-one

Pd/C (10 wt %, 44 mg, 0.041 mmol) was added to 3-{[3-(benzyloxy)phenyl](hydroxy)methyl}-1-(4-chlorophenyl)pyridazin-4(1H)-one (868 mg, 2.07 mmol) in MeOH (30 mL) and allowed to stir under H2 (1 atm). Upon completion the mixture was filtered through Celite, concentrated in vacuo, dried, and purified by flash chromatography (MPLC, 0-10% MeOH-DCM) to afford 3-(3-hydroxybenzyl)-1-phenylpyridazin-4(1H)-one.

LRMS (ESI) calc'd for C17H15N2O2 [M+H]⁺: 279. Found: 279.

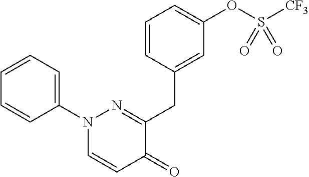

### Step 4. 3-[(4-Oxo-1-phenyl-1,4-dihydropyridazin-3-yl)methyl]phenyl trifluoromethanesulfonate

1,1,1-Trifluoro-N-phenyl-N-[(trifluoromethyl)sulfonyl]methanesulfonamide (1.14 g, 3.19 mmol) and DIPEA (0.607 mL, 3.48 mmol) were added to 3-(3-hydroxybenzyl)-1-phenylpyridazin-4(1H)-one (806 mg, 2.90 mmol) in THF (8 mL). The reaction was allowed to stir at r.t. for 3 days. Additional 1,1,1-trifluoro-N-phenyl-N-[(trifluoromethyl)sulfonyl]methanesulfonamide (1.14 g, 3.19 mmol) and DIPEA (0.607 mL, 3.48 mmol) were added. The temperature was increased to 50° C., and the mixture stirred for an additional 3 days. The reaction mixture was diluted with water, and extracted with EtOAc (3×). The combined organic phases were washed with brine, dried over Na₂SO₄, filtered, and purified by flash chromatography (MPLC, 13-100% EtOAc-Hexanes) to give 3-[(4-oxo-1-phenyl-1,4-dihydropyridazin-3-yl)methyl]phenyl trifluoromethanesulfonate as a yellow oil.

LRMS (ESI) calc'd for C18H14F₃N₂O4S [M+H]⁺: 411. Found: 411.

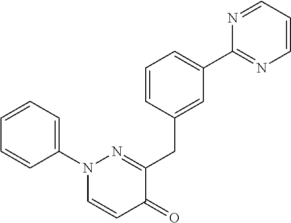

### Step 5. 1-Phenyl-3-[3-(pyrimidin-2-yl)benzyl]-pyridazin-4(1H-one

3-[(4-Oxo-1-phenyl-1,4-dihydropyridazin-3-yl)methyl]phenyl trifluoromethanesulfonate (100 mg, 0.244 mmol), 2-tributylstanylpyrimidine (135 mg, 0.3-66 mmol), LiCl (21 mg, 0.49 mmol), Pd(Ph₃P)₄ (28 mg, 0.024 mmol), CsF (74 mg, 0.487 mmol), and CuI (9 mg, 0.05 mmol) were combined in a 5 mL microwave vial. The vial was evacuated and back-filled with N₂ gas (3×) before adding DMF (4.5 mL). The mixture was allowed to stir at 100° C. for 4 hours. The mixture was then added to an aqueous saturated KF solution and the mixture was stirred for 4 hours. The aqueous phase was extracted with 10% MeOH/DCM (3×) and the combined organic layers were washed with brine, dried over Na₂SO₄, and concentrated in vacuo. Purification by flash chromatography (MPLC, 0-100%, EtOAc-Hexanes followed by 0-10%, EtOAc-MeOH) gave 1-phenyl-3-[3-(pyrimidin-2-yl)benzyl]pyridazin-4(1H)-one.

LRMS (ESI) calc'd for C21H17N4O [M+H]⁺: 342. Found: 342.

### Scheme 22

### Example #670

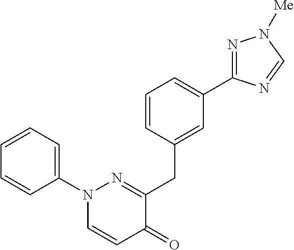

### 3-[3-(1-Methyl-1H-1,2,4-triazol-3-yl)benzyl]-1-phenylpyridazin-4(1H)-one

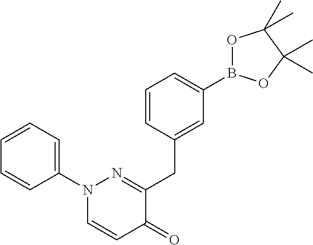

### Step 1. 1-Phenyl-3-[3-(4,4,5,5-tetramethyl-1,3,2-dioxaborolan-2-yl)benzyl]pyridazin-4(1H)-one

3-[(4-Oxo-1-phenyl-1,4-dihydropyridazin-3-yl)methyl]phenyl trifluoromethanesulfonate (Example #669 Step 4, 138 mg, 0.336 mmol), bis(pinacolato)diboron (128 mg, 0.504 mmol), Pd₂(dba)₃ (6 mg, 7 μmol), XPhos (13 mg, 0.027 mmol), and KOAc (99 mg, 1.0 mmol) were combined in a 5 mL microwave vial. The vial was evacuated and back-filled with N₂ gas (3×) before adding 1,4-dioxane (4 mL). The mixture was allowed to stir at 100° C. for 1 hour. EtOAc and water were added, and the aqueous layer was extracted with EtOAc (3×). The combined organic extracts were then washed with brine, dried over Na₂SO₄, concentrated in vacuo, and the residue was purified by flash chromatography (MPLC, 50-100% EtOAc-Hexanes) to afford 1-phenyl-3-[3-(4,4,5,5-tetramethyl-1,3,2-dioxaborolan-2-yl)benzyl]pyridazin-4(1E)-one.

LRMS (ESI) calc'd for C23H25BN2O3 [M+H]⁺: 389. Found: 389.

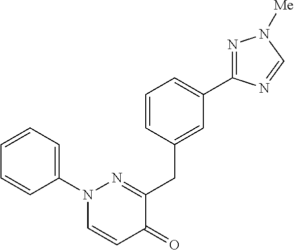

### Step 2. 3-[3-(1-Methyl-1H-1H-1,2,4-triazol-3-yl)benzyl]-1-phenylpyridazin-4(1H-one

1-Phenyl-3-[3-(4,4,5,5-tetramethyl-1,3,2-dioxaborolan-2-yl)benzyl]pyridazin-4(1H)-one (43 mg, 0.11 mmol), 3-bromo-1-methyl-1H-1,2,4-triazole (18 mg, 0.11 mmol), Pd₂(dba)₃ (2 mg, 2 μmol), XPhos (4.1 mg, 8.9 μmol), and Cs₂CO₃ (108 mg, 0.332 mmol) were combined in a 5 mL microwave vial. The vial was evacuated and back-filled with N₂ gas (3×) before adding 1,4-dioxane (2.5 mL), and the mixture was heated to 100° C. for 1 hour. EtOAc and saturated NH₄Cl were added. The aqueous layer was extracted with EtOAc (3×), the combined organic layers were washed with brine, dried over Na₂S₄, concentrated in vacuo and purified by flash chromatography (MPLC, 13-100% EtOAc-Hexanes) followed by reverse phase preparative HPLC (30-100% MeCN—H₂O, 0.05% TFA). Fractions containing the pure compound were washed with saturated NaHCO₃, the aqueous layer was extracted with EtOAc (3×), the organic layer was dried over Na₂SO₄, and the solvent was evaporated in vacuo to afford 3-[3-(1-methyl-1H-1,2,4-triazol-3-yl)benzyl]-1-phenylpyridazin-4(1H)-one.

LRMS (ESI) calc'd for C20H₁₇N₅O [M+H]⁺: 344. Found: 344.

The following example was prepared according to Scheme 22 following a similar procedure described for Example #670, which can be achieved by those of ordinary skill in the art of organic synthesis.

| | | | Exact |
|---|---|---|---|
| Ex- | | IUPAC | Mass |
| ample | Structure | Name | [M + H]⁺ |
| 671 | 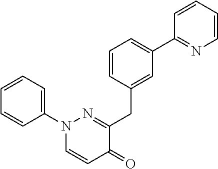 | 1-phenyl- 3-(3- pyridin- 2- ylbenzyl) pyridazin- 4(1H)-one | Calc'd 340, found 340 |

### Scheme 22

### Intermediate #176

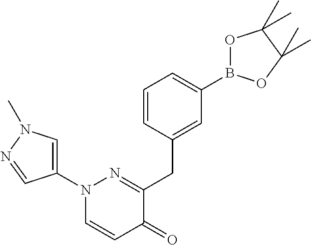

### 1-(1-Methyl-1H-pyrazol-4-yl)-3-[3-(4,4,5,5-tetramethyl-1,3,2-dioxaborolan-2-yl)benzyl]pyridazin-4(1H)-one

### Step 1. 1-(1-Methyl-1H-pyrazol-4-yl)-3-[3-(4,4,5,5-tetramethyl-1,3,2-dioxaborolan-2-yl)benzyl]pyridazin-4(1H)-one

3-(3-Chlorobenzyl)-1-(1-methyl-1H-pyrazol-4-yl)pyridazin-4(1H)-one (Intermediate #127, 730 mg, 2.427 mmol), B₂Pin₂ (925 mg, 3.64 mmol), Pd₂(dba)₃ (45 mg, 0.049 mmol), XPhos (92 mg, 0.194 mmol), and KOAc (715 mg, 7.28 mmol) were combined in a mL microwave vial. The vial was evacuated and back-filled with N₂ gas (3×) before adding 1,4-dioxane (12 mL). The mixture was allowed to stir at 100° C. for 4 hours. The mixture was cooled to ambient temperature and was then filtered through Celite. The solvent was removed in vacuo while loading onto silica. Purification of the residue by flash chromatography (MPLC, 0-10%, MeOH-EtOAc) gave 1-(1-methyl-1H-pyrazol-4-yl)-3-[3-(4,4,5,5-tetramethyl-1,3,2-dioxaborolan-2-yl)benzyl]pyridazin-4(1H)-one.

LRMS (ESI) calc'd for C21H26BN4O3 [M+H]⁺: 393. found 393.

### Scheme 22

### Example #672

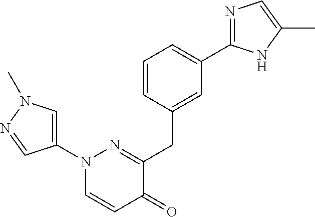

### 3-[3-(5-Methyl-1H-imidazol-2-yl)benzyl]-1-(1-methyl-1H-pyrazol-4-yl)pyridazin-4(1H)-one

### Step 1. 3-[3-(5-Methyl-1H-imidazol-2-yl)benzyl]-1-(1-methyl-1H-pyrazol-4-yl)pyridazin-4(1H)-one

1-(1-Methyl-1H-pyrazol-4-yl)-3-[3-(4,4,5,5-tetramethyl-1,3,2-dioxaborolan-2-yl)benzyl]pyridazin-4(1H)-one (Intermediate #176, 100 mg, 0.255 mmol), 2-bromo-5-methyl-1H-imidazole-(62 mg, 0.382 mmol), Pd₂(dba)₃ (4.7 mg, 5.10 μmol), XPhos (9.7 mg, 0.02 mmol), and Cs₂CO₃ (250 mg, 0.77 mmol) were combined in a 5 mL microwave vial. The vial was evacuated and back-filled with N₂ gas (3×) before adding 1,4-dioxane (4.5 mL). The mixture was allowed to stir at 100° C. for 2 hours. The mixture was filtered through Celite eluting with EtOAc and was then concentrated in vacuo while loading onto silica. Purification by flash chromatography (MPLC, 0-20%, MeOH-EtOAc) gave 3-[3-(5-methyl-1H-imidazol-2-yl)benzyl]-1-(1-methyl-1H-pyrazol-4-yl)pyridazin-4(1H)-one.

LRMS ESI) calc'd for C19H19N6O [M+H]⁺: 347. found 347.

The following examples were prepared according to Scheme 22 following a similar procedure described for Example #672, which can be achieved by those of ordinary skill in the art of organic synthesis.

| | | | Exact |
|---|---|---|---|
| | | IUPAC | Mass |
| Example | Structure | Name | [M + H]⁺ |
| 673 | 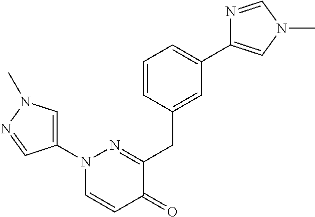 | 3-[3-(1- methyl-1H- imidazol-4- yl)benzyl]-1- (1-methyl-1H- pyrazol-4- yl)pyridazin- 4(1H)-one | Calc'd 347, found 347 |
| 674 | 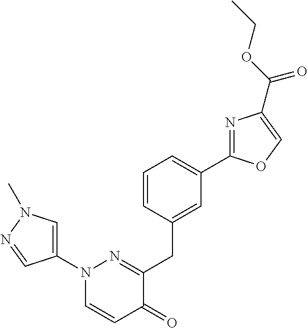 | ethyl 2-(3- {[1-(1- methyl-1H- pyrazol-4-yl)- 4-oxo-1,4- dihydropyridazin- 3-yl]methyl}phenyl)- 1,3- oxazole-4- carboxylate | Calc'd 406, found 406 |
| 675 | 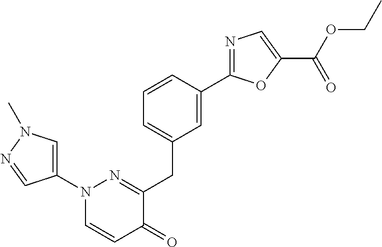 | ethyl 2-(3- {[1-(1- methyl-1H- pyrazol-4-yl)- 4-oxo-1,4- dihydropyridazin- 3- yl]methyl}phenyl)- 1,3- oxazole-5- carboxylate | Calc'd 406, found 406 |
| 676 | 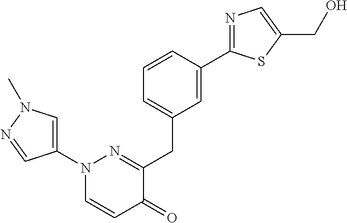 | 3-{3-[5- (hydroxymethyl)- 1,3- thiazol-2- yl]benzyl}-1- (1-methyl-1H- pyrazol-4- yl)pyridazin- 4(1H)-one | Calc'd 380, found 380 |

### Scheme 22

### Example #677

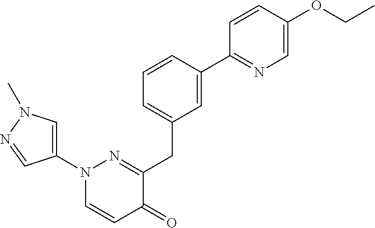

### 3-[3-(5-Ethoxypyridin-2-yl)benzyl]-1-(1-methyl-1H-pyrazol-4-ylpyridazin-4(1H)-one

### Step 1. 3-[3-(5-Ethoxypyridin-2-yl)benzyl]-1-(1-methyl-1H-pyrazol-4-yl)pyridazin-4(1H)-one

1-(1-Methyl-1H-pyrazol-4-yl)-3-[3-(4,4,5,5-tetramethyl-1,3,2-dioxaborolan-2-yl)benzyl]pyridazin-4(1H)-one (Intermediate #176, 68 mg, 0.173 mmol), PdCl₂(dppf).DCM adduct (14 mg, 0.017 mmol), 2 N Na₂CO₃ (0.175 mL, 0.35 mmol) and 2-bromo-5-ethoxypyridine 53 mg, 0.260 mmol) were taken up in 1,4-dioxane (0.85 mL). The flask was evacuated and back-filled with N₂ (x3) before stirring at 100° C. for 4 hours. Room temperature was attained and the solvent removed in vacuo while loading onto silica. The residue was purified by flash chromatography (MPLC, 0-15% MeOH-EtOAc) followed by reverse phase preparative HPLC (20-40% MeCN—H₂O, 0.05% TFA). Product fractions were neutralized directly with saturated NaHCO₃ and extracted into EtOAc (x2). The combined organic extracts were dried over MgSO₄, filtered and concentrated in vacuo. The residue was triturated in hexanes to give 3-[3-(5-ethoxypyridin-2-yl)benzyl]-1-(1-methyl-1H-pyrazol-4-yl)pyridazin-4(1H)-one as a white solid.

LRMS (ESI) calc'd for C22H22N5O2 [M+H]⁺: 388. found 388.

### Scheme 23

### Intermediate #-177

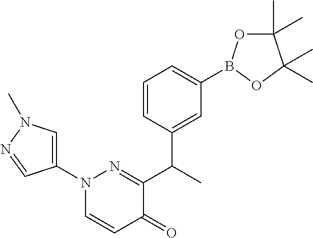

### rac-(1-Methyl-1H-pyrazol-4-yl)-3-{1-[3-(4,4,5,5-tetramethyl-1,3,2-dioxaborolan-2-yl)phenyl]ethyl}pyridazin-4(1H)-one

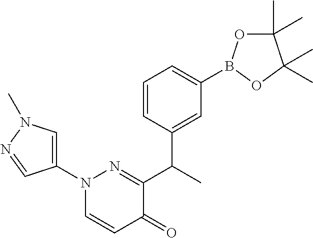

### Step 1. rac-1-(1-Methyl-1H-pyrazol-4-yl)-3-{1-[3-(4,4,5,5-tetramethyl-1,3,2-dioxaborolan-2-yl)phenyl]ethyl}pyridazin-4(1H)-one

rac-1-(1-Methyl-1H-pyrazol-4-yl)-3-{1-[3-(4,4,5,5-tetramethyl-1,3,2-dioxaborolan-2-yl)phenyl]ethyl}pyridazin-4(1H)-one was prepared from rac-3-[1-(3-chlorophenyl)ethyl]-1-(1-methyl-1H-pyrazol-4-yl)pyridazin-4(1H)-one (Intermediate #145) according to the procedure described for 1-(1-methyl-1H-pyrazol-4-yl)-3-[3-(4,4,5,5-tetramethyl-1,3,2-dioxaborolan-2-yl)benzyl]pyridazin-4(1H)-one (Intermediate #176).

LRMS (ESI) calc'd for C22H28BN4O3 [M+H]⁺: 407. Found: 407.

### Scheme 23

### Intermediate #178

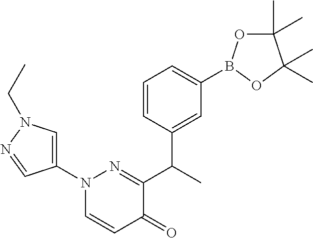

### rac-1-(1-Ethyl-1H-pyrazol-4-yl)-3-{1-[3-(4,4,5,5-tetramethyl-1,3,2-dioxaborolan-2-yl)phenyl]ethyl}pyridazin-4(1H)-one

### Step 1; rac-1-(1-Ethyl-1H-pyrazol-4-yl)-3-{1-[3-(4,4,5,5-tetramethyl-1,3,2-dioxaborolan-2-yl)phenyl]ethyl}pyridazin-4(1H)-one

rac-1-(1-Ethyl-1H-pyrazol-4-yl)=3-{1-[3-(4,4,5,5-tetramethyl-1,3,2-dioxaborolan-2-yl)phenyl]ethyl}pyridazin-4(1H)-one was prepared from rac-3-[1-(3-chlorophenyl)ethyl]-1-(1-ethyl-1H-pyrazol-4-yl)pyridazin-4(1H)-one (Intermediate #146 according to the procedure described for 1-(1-methyl-1H-pyrazol-4-yl)-3-[3-(4,4,5,5-tetramethyl-1,3,2-dioxaborolan-2-yl)benzyl]pyridazin-4(1H)-one (Intermediate #176).

LRMS (ESI) calc'd for C23H30BN4O3 [M+H]⁺: 421. Found: 421.

### Scheme 23

### Intermediate #179

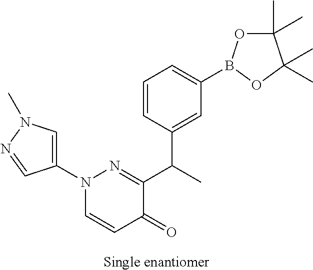

### 1-(1-Methyl-1H-pyrazol-4-yl)-3-{(1R or S)-1-[3-(4,4,5,5-tetramethyl-1,3,2-dioxaborolan-2-yl)phenyl]ethyl}pyridazin-4(1H)-one

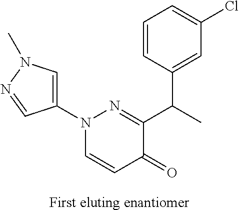

### Step 1. 3-[1R or S)-1-(3-Chlorophenyl)ethyl]-1-(1-methyl-1H-pyrazol-4-yl)pyridazin-4(1H)-one (first eluting enantiomer)

The enantiomers of rac-3-[1-(3-chlorophenyl)ethyl]-1-(1-methyl-1H-pyrazol-4-yl)pyridazin-4(1H)-one (Intermediate #145) were separated by normal phase preparative HPLC (column: Astec Chirobiotic T, mobile phase 30% heptane in EtOH) and the first eluting enantiomer was subsequently shown to yield the more active final product (by converting each enantiomer to the boronic ester [Step 2] and synthesizing known compounds Examples #201 and 202).

LRMS (ESI) calc'd for C16H16ClN4O [M+H]⁺: 315. Found: 315.

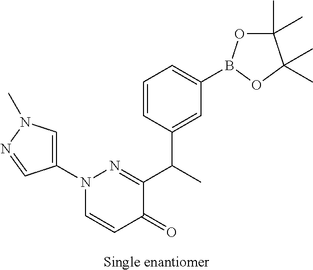

### Step 2. 1-(1-Methyl-1H-pyrazol-4-yl)-3-{(1R or S)-1-[3-(4,4,5,5-tetramethyl-1, 3,2-dioxaborolan-2-yl)phenyl]ethyl}pyridazin-4(1H)-one

1-(1-Methyl-1H-pyrazol-4-yl)-3-{(1R or S)-1-[3-(4,4,5,5-tetramethyl-1,3,2-dioxaborolan-2-yl)phenyl]ethyl}pyridazin-4(1-H)-one was prepared from 3-[(1R or S)-1-(3-chlorophenyl)ethyl]-1-(1-methyl-1H-pyrazol-4-yl)pyridazin-4(1H)-one (first eluting enantiomer) according to the procedure described for 1-(1-methyl-1H-pyrazol-4-yl)-3-[3-(4,4,5,5-tetramethyl-1,3,2-dioxaborolan-2-yl)benzyl]pyridazin-4(1H)-one (Intermediate #176).

LRMS (ESI) calc'd for C22H28ClN4O3 [M+H]⁺: 407. Found: 407.

### Scheme 23

### Example #678

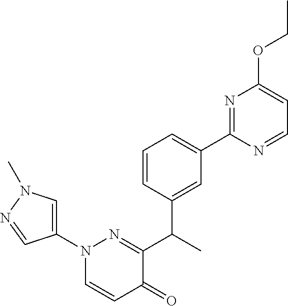

### rac-3-{1-[3-(4-Ethoxypyrimidin-2-yl)phenyl]ethyl}-1-(1-methyl-1H-pyrazol-4-yl)pyridazin-4(1H)-one

### Step 1. rac-3-{1-[3-(4-Ethoxypyrimidin-2-yl)phenyl]ethyl}-1-(1-methyl-1H-pyrazol-4-yl)pyridazin-4(1H)-one

rac-3-({1-[3-(4-Ethoxypyrimidin-2-yl)phenyl]ethyl}-1-(1-methyl-1H-pyrazol-4-yl)pyridazin-4(1H)-one was prepared from rac-1-(1-methyl-1H-pyrazol-4-yl)-3-{1-[3-(4,4,5,5-tetramethyl-1,3,2-dioxaborolan-2-yl)phenyl]ethyl}pyridazin-4(1H)-one (Intermediate #177) and 2-chloro-4-ethoxypyrimidine (Intermediate #206) according to the procedure described for 3-[3-(5-methyl-1H-imidazol-2-yl)benzyl]-1-(1-methyl-1H-pyrazol-4-yl)pyridazin-4(1H)-one (Example #672).

LRMS (ESI) calc'd for C22H23N6O2 [M+H]⁺: 403. Found: 403

The following example was prepared from Intermediate #177 and Intermediate #207 according to Scheme 23 following a similar procedure described for Example #672, which can be achieved by those of ordinary skill in the art of organic synthesis.

| | | | Exact |
|---|---|---|---|
| | | IUPAC | Mass |
| Example | Structure | Name | [M + H]⁺ |
| 679 | 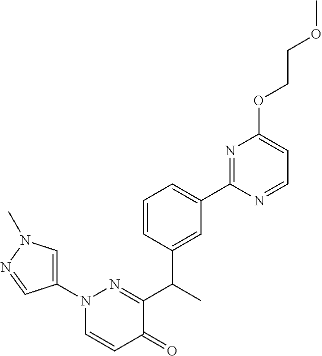 | rac-3-(1-{3- [4-(2- methoxyethoxy) pyrimidin- 2- yl]phenyl}ethyl)- 1-(1- methyl-1H- pyrazol-4- yl)pyridazin- 4(1H)-one | Calc'd 433, found 433 |

Procedures for the preparation of the aryl halides-(Intermediates #206-207) used in the synthesis of Examples #678-679 are shown below.

### Scheme 23

### Examples #680 and 681

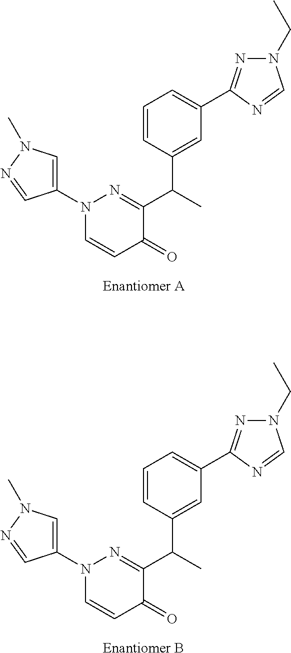

### 3-{(15 or R)-1-[3-(1-Ethyl-1H-1,2,4-triazol-3-yl)phenyl]ethyl}-1-(1-methyl-1H-pyrazol-4-yl)pyridazin-4(1H)-one (Enantiomer A) 3-{(1R or S)-1-[3-(1-ethyl-1H-1,2,4-triazol-3-yl)phenyl]ethyl}-1-(1-methyl-1H-pyrazol-4-yl)pyridazin-4(1H)-one (Enantiomer B)

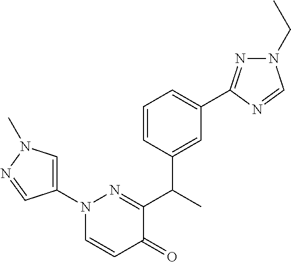

### Step 1. rac-3-{1-[3-(1-Ethyl-1H-1,2,4-triazol-3-yl)phenyl]ethyl}-1-(1-methyl-1H-pyrazol-4-yl)pyridazin-4(1H)-one

rac-3-{1-[3-(1-Ethyl-1H-1,2,4-triazol-3-yl)phenyl]ethyl}-1-(1-methyl-1H-pyrazol-4-yl)pyridazin-4(1H)-one was prepared from rac-1-(1-methyl-1H-pyrazol-4-yl)-3-{1-[3-(4,4,5,5-tetramethyl-1,3,2-dioxaborolan-2-yl)phenyl]ethyl}pyridazin-4(1H)-one (Intermediate #177) and 3-bromo-1-ethyl-1H-1,2,4-triazole (Intermediate #204) according to the procedure described for 3-[3-(5-methyl-1H-imidazol-2-yl)benzyl]—(1-methyl-1-pyrazol-4-yl)pyridazin-4(1H)-one (Example #672).

LRMS (ESI) calc'd for C20H22N7O [M+H]⁺: 376. found 376.

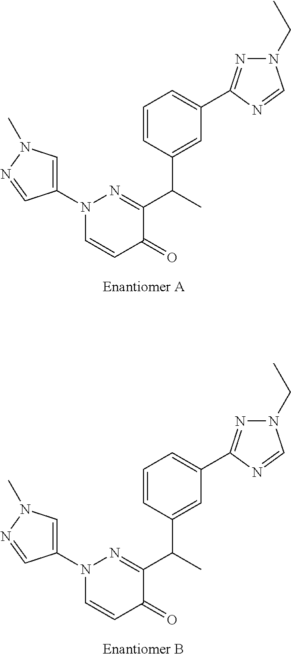

### Step 2. 3-{(1S or R)-1-[3-(1-Ethyl-1H-1,2,4-triazol-3-yl)phenyl]ethyl}-1-(1-methyl-1H-pyrazol-4-yl)pyridazin-4(1H)-one (Enantiomer A) and 3-{(1R or S)-1-[3-(1-ethyl-1H-1,2,4-triazol-3-yl)phenyl]ethyl}-1-(1-methyl-1H-pyrazol-4-yl)pyridazin-4(1H)-one (Enantiomer B)

rac-3-{1-[3-(1-Ethyl-1H-1,2,4-triazol-3-yl)phenyl]ethyl}-1-(1-methyl-1H-pyrazol-4-yl)pyridazin-4(1H)-one (85 mg, 0.226 mmol) was resolved by SFC (Berger Multigram II SFC, column: ChiralPak: AD-H 50×250 mm, mobile phase: 25% MeOH/75% CO₂₍ₗ₎, flow rate: 230 mL/min) to give 3-{(1S or R)-1-[3-(1-ethyl-1H-1,2,4-triazol-3-yl)phenyl]ethyl}-1-(1-methyl-1H-pyrazol-4-yl)pyridazin-4(1H)-one (Enantiomer A, Example #680) and 3-{(1R or S)-1-[3-(1-ethyl-1H-1,2,4-triazol-3-yl)phenyl]ethyl}-1-(1-methyl-1H-pyrazol-4-yl)pyridazin-4(1H)-one (Enantiomer B, Example #681).

### Example #680

LRMS (ESI) calc'd for C20H22N7O [M+H]⁺: 376. found 376.

### Example #681

LRMS (ESI) calc'd for C20H22N7O [M+H]⁺: 376. found 376.

The following examples were prepared from Intermediates #177-178 according to Scheme 23 following similar procedures described for Examples #680-681, which can be achieved by those of ordinary skill in the art of organic synthesis.

| | | | Exact |
|---|---|---|---|
| | | IUPAC | Mass |
| Example | Structure | Name | [M + H]⁺ |
| 682 | 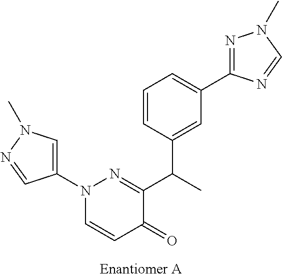 | 1-(1-methyl- 1H-pyrazol-4- yl)-3-{(1S or R)-1-[3-(1- methyl-1H- 1,2,4-triazol- 3- yl)phenyl]ethyl} pyridazin- 4(1H)-one | Calc'd 362, found 362 |
| 683 | 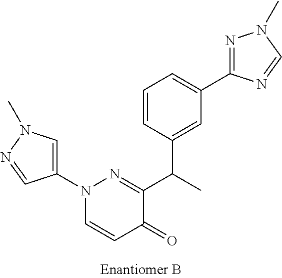 | 1-(1-methyl- 1H-pyrazol-4- yl)-3-{(1R or S)-1-[3-(1- methyl-1H- 1,2,4-triazol- 3- yl)phenyl]ethyl} pyridazin- 4(1H)-one | Calc'd 362, found 362 |
| 684 | 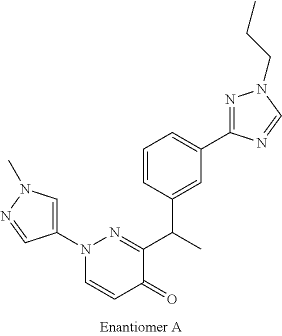 | 1-(1-methyl- 1H-pyrazol-4- yl)-3-{(1S or R)-1-[3-(1- propyl-1H- 1,2,4-triazol-3- yl)phenyl]ethyl} pyridazin- 4(1H)-one | Calc'd 390, found 390 |
| 685 | 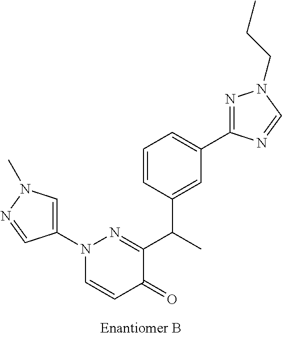 | 1-(1-methyl- 1H-pyrazol-4- yl)-3-{(1R or S)-1-[3-(1- propyl-1H- 1,2,4-triazol- 3- yl)phenyl]ethyl} pyridazin- 4(1H)-one | Calc'd 390, found 390 |
| 686 | 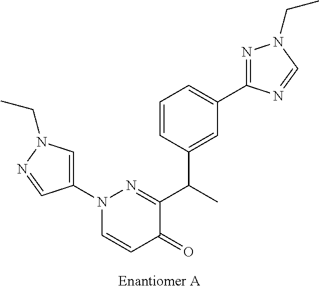 | 1-(1-ethyl-1H- pyrazol-4-yl)- 3-{(1S or R)- 1-[3-(1-ethyl- 1H-1,2,4- triazol-3- yl)phenyl]ethyl} pyridazin- 4(1H)-one | Calc'd 390, found 390 |
| 687 | 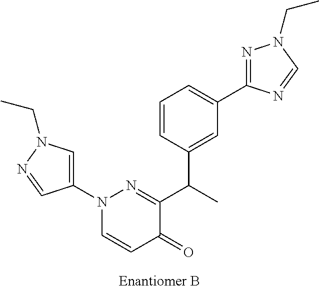 | 1-(1-ethyl-1H- pyrazol-4-yl)- 3-{(1R or S)- 1-[3-(1-ethyl- 1H-1,2,4- triazol-3- yl)phenyl]ethyl} pyridazin- 4(1H)-one | Calc'd 390, found 390 |
| 688 | 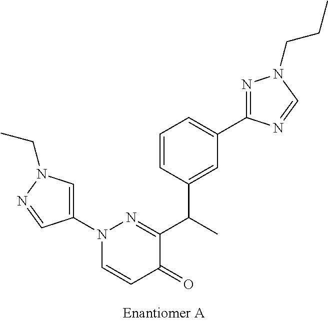 | 1-(1-ethyl-1H- pyrazol-4-yl)- 3-{(1S or R)- 1-[3-(1- propyl-1H- 1,2,4-triazol- 3- yl)phenyl]ethyl} pyridazin- 4(1H)-one | Calc'd 404, found 404 |
| 689 | 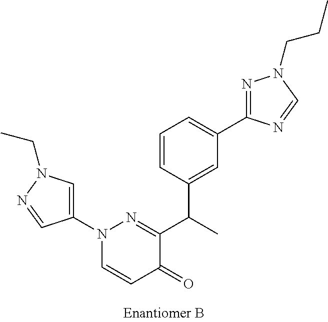 | 1-(1-ethyl-1H- pyrazol-4-yl)- 3-{1R or S)- 1-[3-(1- propyl-1H- 1,2,3-triazol-3- yl)phenyl]ethyl} pyridazin- 4(1H)-one | Calc'd 404, found 404 |

Procedures for the preparation of the aryl halides (Intermediates #204-205) used in the synthesis of Examples #680-681 and 684-689 are shown below.

### Scheme 23

### Example #690

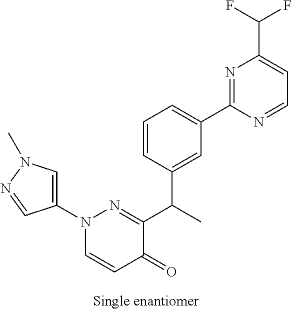

### 3-[(1R or S)-1-{3-[4-(Difluoromethyl)pyrimidin-2-yl]phenyl}ethyl]-1-(1-methyl-1H-pyrazol-4-yl)pyridazin-4(1H)-one (Enantiomer B)

### Step 1. 3-[(1R or S)-1-{3-[4-(Difluoromethyl)pyrimidin-2-yl]phenyl}ethyl]-1-(1-methyl-1H-pyrazol-4-yl)pyridazin-4(1H)-one

3-[(1R or S)-1-{3-[4-(Difluoromethyl)pyrimidin-2-yl]phenyl}ethyl]-1-(1-methyl-TH-pyrazol-4-yl)pyridazin-4(1H)-one was prepared from 1-(1-methyl-1H-pyrazol-4-yl)-3-{(1R or S)-1-[3-(4,4,5,5-tetramethyl-1,3,2-dioxaborolan-2-yl)phenyl]ethyl}pyridazin-4(1H)-one (Intermediate #179) and 2-chloro-4-(difluoromethyl)pyrimidine (Intermediate #210) according to the procedure described for 3-[3-(5-methyl-1H-imidazol-2-yl)benzyl]-1-(1-methyl-1H-pyrazol-4-yl)pyridazin-4(1H)-one (Example #672).

LRMS (ESI) calc'd for C21H19F₂N6O [M+H]⁺: 409. Found: 409.

The following examples were prepared according to Scheme 23 following a similar procedure described for Example #672, which can be achieved by those of ordinary skill in the art of organic synthesis.

| | | | Exact |
|---|---|---|---|
| | | IUPAC | Mass |
| Example | Structure | Name | [M + H]⁺ |
| 691 | 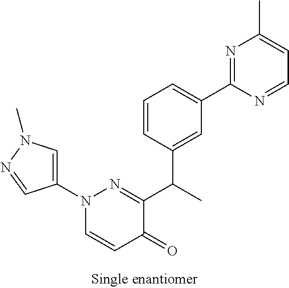 | 1-(1-methyl- 1H-pyrazol-4- yl)-3-{(1R or S)-1-[3-(4- methylpyrimidin- 2- yl)phenyl]ethyl} pyridazin- 4(1H)-one | Calc'd 373, found 373 |
| 692 | 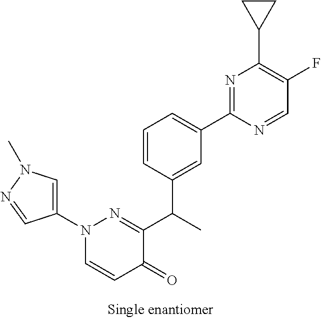 | 3-{(1R or S)- 1-[3-(4- cyclopropyl- 5- fluoropyrimidin- 2- yl)phenyl]ethyl}- 1-(1- methyl-1H- pyrazol-4- yl)pyridazin- 4(1H)-one | Calc'd 417, found 417 |
| 693 | 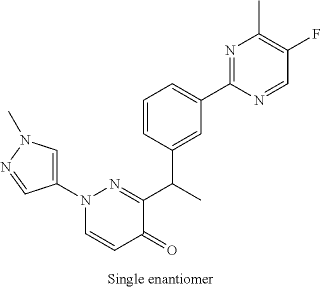 | 3-{(1R or S)- 1-[3-(5- fluoro-4- methylpyrimidin- 2- yl)phenyl]ethyl}- 1}-1-(1- methyl-1H- pyrazol-4- yl)pyridazin- 4(1H)-one | Calc'd 391, found 391 |
| 694 | 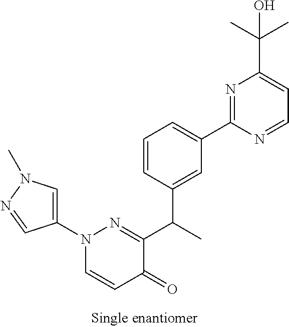 | 3-[(1R or S)- 1-{3-[4-(2- hydroxypropan- 2- yl)pyrimidin- 2- yl]phenyl}ethyl]- 1-(1- methyl-1H- pyrazol-4- yl)pyridazin- 4(1H)-one | Calc'd 417, found 417 |

The following intermediate was prepared according to Scheme 23 following a similar procedure described for Example #672, which can be achieved by those of ordinary skill in the art of organic synthesis.

| | | | Exact |
|---|---|---|---|
| | | IUPAC | Mass |
| Intermediate | Structure | Name | [M + H]⁺ |
| 180 | 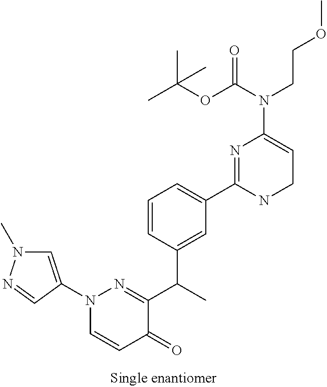 | tert-butyl (2- methoxyethyl) [2-(3-{(1R or S)-1-[1-(1- methyl-1H- pyrazol-4-yl)- 4-oxo-1,4- dihydropyridazin-3- yl]ethyl}phenyl) pyrimidin- 4- yl]carbamate | Calc'd 532, found 532 |

Procedures for the preparation of the aryl halides (Intermediates #208-211) used in the synthesis of Examples #690, 692 and 694 and Intermediate #180 are shown below.

### Scheme 23

### Example #695

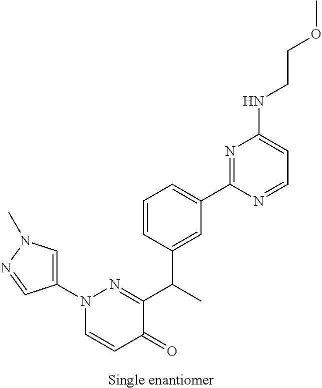

### 3-[(1R or S)-1-(3-{4-[(2-Methoxyethyl)amino]pyrimidin-2-yl}phenyl)ethyl]-1-(1-methyl-1H-pyrazol-4-yl)pyridazin-4(1H)-one

### Step 1. 3-[(1R or S)-1-(3-{4-1(2-Methoxyethyl)aminopyrimidin-2-yl}-phenyl)ethyl]-1-(1-methyl-1H-pyrazol-4-yl)pyridazin-4(1H)-one

To a solution of tert-butyl (2-methoxyethyl)[2-(3-{(1R or S)-1-[1-(1-methyl-1H-pyrazol-4-yl)-4-oxo-1,4-dihydropyridazin-3-yl]ethyl}phenyl)pyrimidin-4-yl]carbamate (Intermediate #180, 20 mg 0.038 mmol) in DCM (2 mL) was added TFA (0.529 mL 6.266 mmol). The reaction was stirred at r.t. for about 4 hrs. The solvent was removed in vacuo and the residue purified by reverse phase preparative HPLC (0-80% MeCN—H₂O, 0.05% TFA). Product fractions were eluted through PL-HCO₃ cartridges (Stratospheres™, 0.9 mmol) to remove the TFA. The filtrate was frozen and freeze dried to afford 3-[(1R or S)-1-(3-{4-[(2-methoxyethyl)amino]pyrimidin-2-yl}phenyl)ethyl]-1-(1-methyl-1H-pyrazol-4-yl)pyridazin-4(1H)-one.

LRMS (ESI) calc'd for C23H26N₇O2 [M+H]⁺: 432. Found: 432.

### Scheme 24

### Example #696

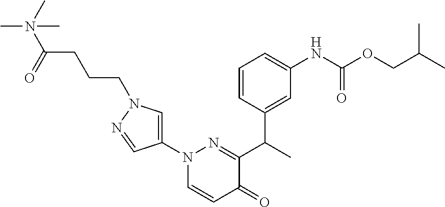

### 2-Methylpropyl {3-[(1-{1-[4-(dimethylamino)-4-oxobutyl]-1H-pyrazol-4-yl}-4-oxo-1,4-dihydropyridazin-3-yl)methyl]phenyl}carbamate

### Step 1. 2-Methylpropyl{3-[(1-{1-[4-dimethylamino)-4-oxobutyl]-1H-pyrazol-4-yl}-4-oxo-4,4-dihydropyridazin-3-yl)methyl]phenyl}carbamate

To 4-{4-[3-(3-{[(2-methylpropoxy)carbonyl]amino}benzyl)-4-oxopyridazin-1(4H)-yl]-1H-pyrazol-1-yl}butanoic acid (Example #180, 40 mg, 0.088 mmol) in a vial was added CHCl₃ (441 μL), DIPEA (62 μL, 0.35 mmol), dimethylamine (88 μL, 0.18 mmol) and TBTU (42.3 mg, 0.132 mmol). The mixture was stirred at r.t. Upon complete conversion the mixture was concentrated in vacuo and the residue was purified by flash chromatography (MPLC, 1:1 DCM/ether followed by 0-15% MeOH). The product was further purified by reverse phase preparative HPLC (35-70% MeCN—H₂O, 0.05% TFA) to afford 2-methylpropyl {3-[(1-{1-[4-(dimethylamino)-4-oxobutyl]-1H-pyrazol-4-yl}-4-oxo-1,4-dihydropyridazin-3-yl)methyl]phenyl}carbamate.

LRMS (ESI) calc'd for C25H33N6O4 [M+H]⁺: 481. Found: 481.

The following examples were prepared from Examples #180-182 according to Scheme 24 following a similar procedure described for Example #696, which can be achieved by those of ordinary skill in the art of organic synthesis.

| | | | Exact |
|---|---|---|---|
| | | | Mass |
| Example | Structure | IUPAC Name | [M + H]⁺ |
| 697 | 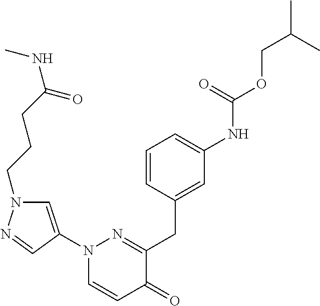 | 2-methylpropyl {3- [(1-{1-[4- (methylamino)-4- oxobutyl]-1H- pyrazol-4-yl}-4- oxo-1,4- dihydropyridazin-3- yl)methyl]phenyl} carbamate | Calc'd 467, found 467 |
| 698 | 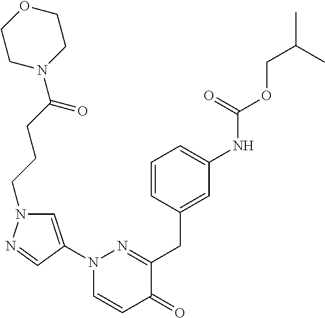 | 2-methylpropyl [3- ({1-[1-(4- morpholin-4-yl-4- oxobutyl)-1H- pyrazol-4-yl]-4- found oxo-1,4- dihydropyridazin-3- yl}methyl)phenyl] carbamate | Calc'd 523, found 523 |
| 699 | 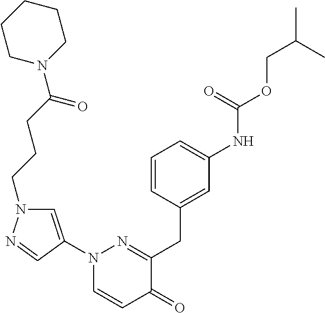 | 2-methylpropyl [3- ({4-oxo-1-[1-(4- oxo-4-piperidin-1- ylbutyl)-1H- pyrazol-4-yl]-1,4- dihydropyridazin-3- yl}methyl)phenyl] carbamate | Calc'd 521, found 521 |
| 700 | 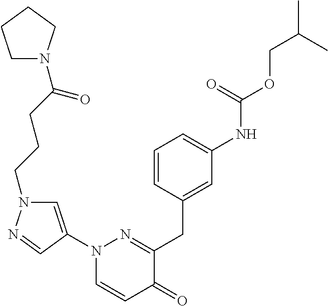 | 2-methylpropyl [3- ({4-oxo-1-[1-(4- oxo-4-pyrrolidin-1- ylbutyl)-1H- pyrazol-4-yl]-1,4- dihydropyridazin-3- yl}methyl)phenyl] carbamate | Calc'd 507, found 507 |
| 701 | 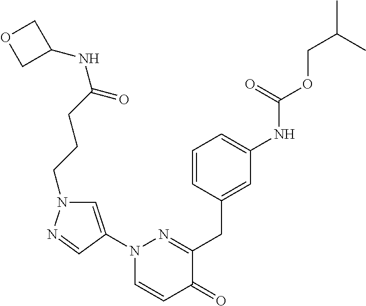 | 2-methylpropyl {3- [(1-{1-[4-(oxetan- 3-ylamino)-4- oxobutyl]-1H- pyrazol-4-yl}-4- oxo-1,4- dihydropyridazin-3- yl)methyl]phenyl} carbamate | Calc'd 509, found 509 |
| 702 | 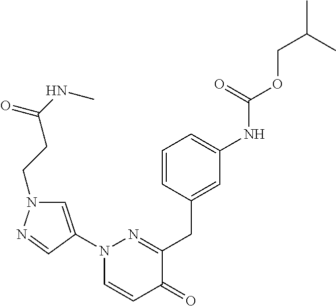 | 2-methylpropyl {3- [(1-{1-[3- (methylamino)-3- oxopropyl]-1H- pyrazol-4-yl}-4- oxo-1,4- dihydropyridazin-3- yl)methyl]phenyl} carbamate | Calc'd 453, found 453 |
| 703 | 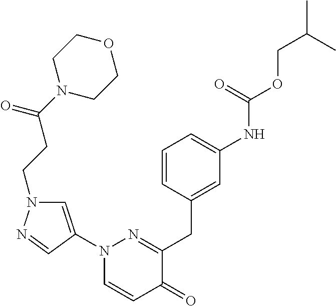 | 2-methylpropyl [3- ({1-[1-(3- morpholin-4-yl-3- oxopropyl-1H- pyrazol-4-yl]-4- oxo-1,4- dihydropyridazin-3- yl}methyl)phenyl] carbamate | Calc'd 509, found 509 |
| 704 | 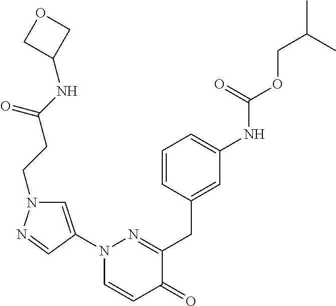 | 2-methylpropyl {3- [(1-{1-[3-(oxetan- 3-ylamino)-3- oxopropyl]-1H- pyrazol-4-yl}-4- oxo-1,4- dihydropyridazin-3- yl)methyl]phenyl} carbamate | Calc'd 495, found 495 |
| 705 | 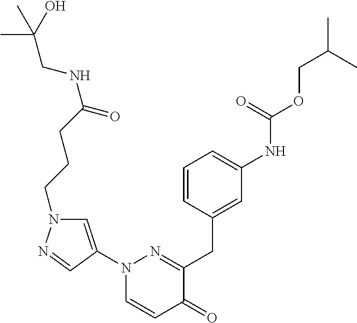 | 2-methylpropyl (3- {[1-(1-{4-[(2- hydroxy-2- methylpropyl)amino]- 4-oxobutyl}-1H- pyrazol-4-yl)-4- oxo-1,4- dihydropyridazin-3- yl]methyl}phenyl) carbamate | Calc'd 525, found 525 |
| 706 | 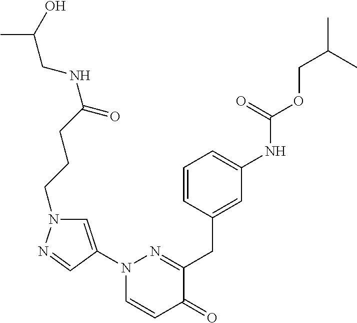 | rac-2-methylpropyl (3-{[1-(1-{4-[(2- hydroxypropyl)amino]- 4-oxobutyl}- 1H-pyrazol-4-yl)-4- oxo-1,4- dihydropyridazin-3- yl]methyl}phenyl) carbamate | Calc'd 511, found 511 |

### Scheme 25

### Example #707

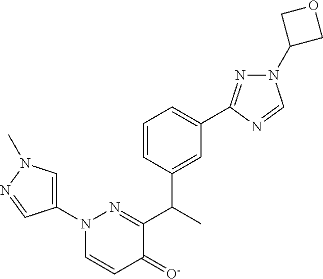

### 1-(1-Methyl-1H-pyrazol-4-yl)-3-{3-[1-(oxetan-3-yl)-1H-1,2,4-triazol-3-yl]benzyl}pyridazin-4(1H)-one

### Step 1. 1-(1-Methyl-1H-pyrazol-4-yl)-3-{3-[1-(oxetan-3-yl)-1H-1,2,4-triazol-3-yl]benzyl}pyridazin-4(1H)-one

1-(1-Methyl-1H-pyrazol-4-yl)-3-[3-(1H-1,2,4-triazol-3-yl)benzyl]pyridazin-4(1H)-one hydrochloride (Example #186, 75 mg, 0.203 mmol), 3-iodooxetane (37.3 mg, 0.203 mmol), and Cs₂CO₃ (145 mg, 0.446 mmol) were combined in 5 mL microwave vial. DMF (3 mL) was added and the mixture was heated to 150° C. for 30 minutes under microwave irradiation (Biotage, Initiator). EtOAc (50 mL) and saturated NH₄Cl (50 mL) were added. The aqueous phase was extracted with further portions of EtOAc (x3) and the combined organic extracts were washed with brine, dried over Na₂SO₄, filtered and concentrated in vacuo. The residue was purified by reverse phase preparative HPLC (5-50% MeCN—H₂O, 0.1% TFA). The product fractions were combined and the solvent was removed under reduced pressure. The resulting residue was re-suspended in acetonitrile (5 mL) and the freebase was liberated using PL-HCO₃ cartridges (Stratospheres™, 0.9 mmol) to give 1-(1-methyl-1H-pyrazol-4-yl)-3-{3-[1-(oxetan-3-yl)-1H-1,2,4-triazol-3-yl]benzyl}pyridazin-4(1H)-one.

LRMS (ESI) calc'd for C20H20N₇O2 [M+H]⁺: 390. found 390.

The following examples were prepared according to Scheme 25 following a similar procedure described for Example #707, which can be achieved by those of ordinary skill in the art of organic synthesis.

| | | | Exact |
|---|---|---|---|
| | | | Mass |
| Example | Structure | IUPAC Name | [M + H]⁺ |
| 708 | 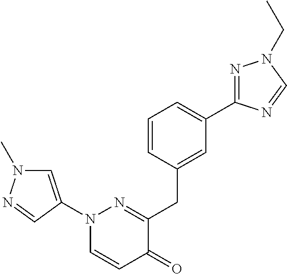 | 3-[3-(1-ethyl- 1H-1,2,4- triazol-3- yl)benzyl]-1-(1- methyl-1H- pyrazol-4- yl)pyridazin- 4(1H)-one | Calc'd 362, found 362 |
| 709 | 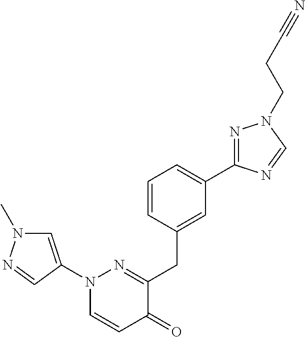 | 3-[3-(3-{[1-(1- methyl-1H- pyrazol-4-yl)-4- oxo-1,4- dihydropyridazin-3- yl]methyl}phenyl)- 1H-1,2,4- triazol-1- yl]propanenitrile | Calc'd 387, found 387 |
| 710 | 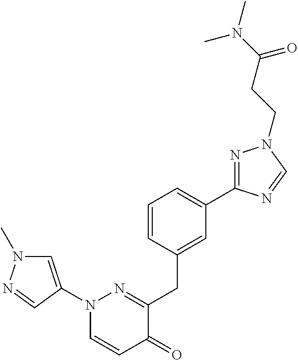 | N,N-dimethyl- 3-[3-(3-{[1-(1- methyl-1H- pyrazol-4-yl)-4- oxo-1,4- dihydropyridazin-3- yl]methyl}phenyl)- 1H-1,2,4- triazol-1- yl]propanamide | Calc'd 433, found 433 |
| 711 | 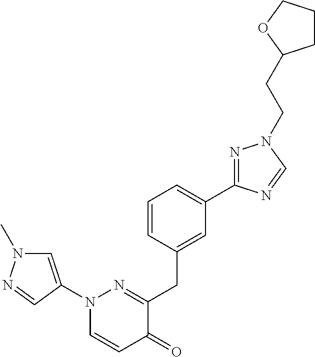 | rac-1-(1- methyl-1H- pyrazol-4-yl)-3- (3-{1-[2- (tetrahydrofuran- 2-yl)ethyl]-1H- 1,2,4-triazol-3- yl}benzyl)pyridazin- 4(1H)-one | Calc'd 432, found 432 |
| 712 | 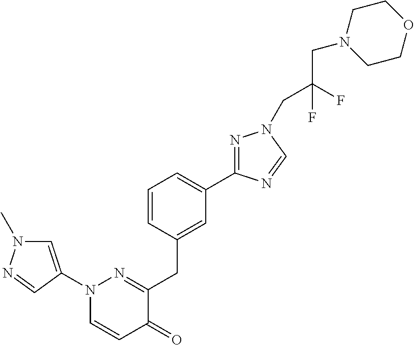 | 3-{3-[1-(2,2- difluoro-3- morpholin-4- ylpropyl)-1H- 1,2,4-triazol-3- yl]benzyl}-1-(1- methyl-1H- pyrazol-4- yl)pyridazin- 4(1H)-one | Calc'd 497, found 497 |
| 713 | 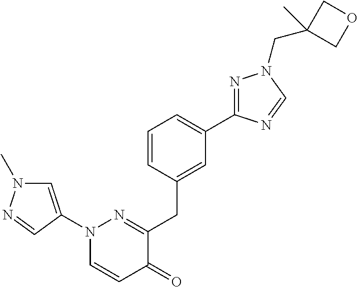 | 3-(3-{1-[(3- methyloxetan-3- yl)methyl]-1H- 1,2,4-triazol-3- yl}benzyl)-1-(1- methyl-1H- pyrazol-4- yl)pyridazin 4(1H)-one | Calc'd 418, found 418 |
| 714 | 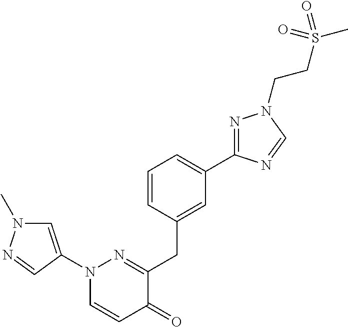 | 1-(1-methyl- 1H-pyrazol-4- yl)-3-(3-{1-[2- (methylsulfonyl) ethyl]-1H- 1,2,4-triazol-3- yl}benzyl)pyridazin- 4(1H)-one | Calc'd 440, found 440 |
| 715 | 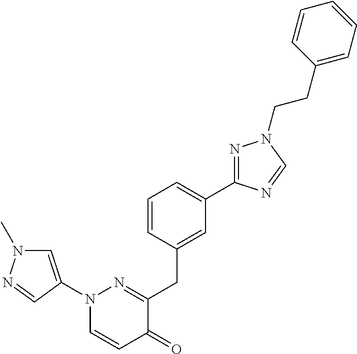 | 1-(1-methyl- 1H-pyrazol-4- yl)-3-{3-[1-(2- phenylethyl)- 1H-1,2,4- triazol-3- yl]benzyl}pyridazin- 4(1H)-one | Calc'd 438, found 438 |
| 716 | 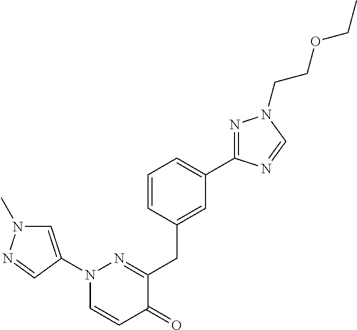 | 3-{3-[1-(2- ethoxyethyl)- 1H-1,2,4- triazol-3- yl]benzyl}-1-(1- methyl-1H- pyrazol-4- yl)pyridazin 4(1H)-one | Calc'd 406, found 406 |
| 717 | 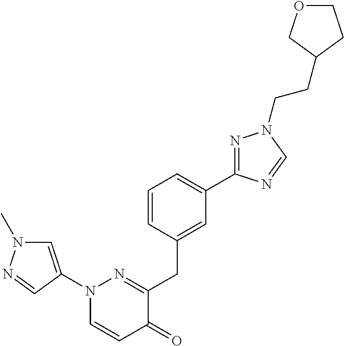 | rac-1-(1- methyl-1H- pyrazol-4-yl)-3- (3-{1-[2- (tetrahydrofuran- 3-yl)ethyl]-1H- 1,2,4-triazol-3- yl}benzyl)pyridazin- 4(1H)-one | Calc'd 432, found 432 |
| 718 | 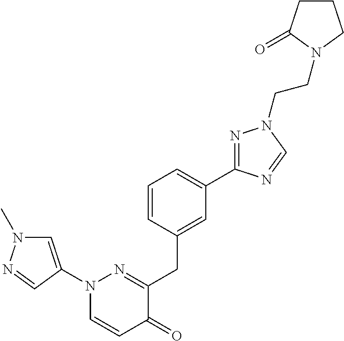 | 1-(1-methyl- 1H-pyrazol-4- yl)-3-(3-{1-[2- (2- oxopyrrolidin 1-yl)ethyl-1H- 1,2,4-triazol-3- yl}benzyl)pyridazin- 4(1H)-one | Calc'd 445, found 445 |
| 719 | 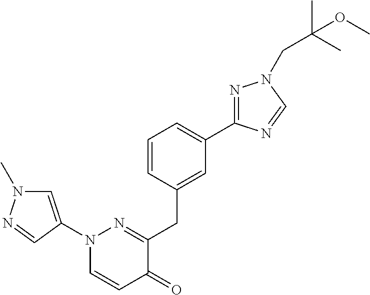 | 3-{3-[1-(2- methoxy-2- methylpropyl)- 1H-1,2,4- triazol-3- yl]benzyl}-1-(1- methyl-1H- pyrazol-4- yl)pyridazin- 4(1H)-one | Calc'd 420, found 420 |
| 720 | 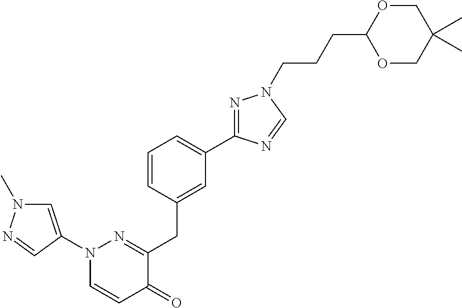 | 3-(3-{1-[3-(5,5- dimethyl-1,3- dioxan-2- yl)propyl]-1H- 1,2,4-triazol-3- yl}benzyl)-1-(1- methyl-1H- pyrazol-4- yl)pyridazin- 4(1H)-one | Calc'd 490, found 490 |
| 721 | 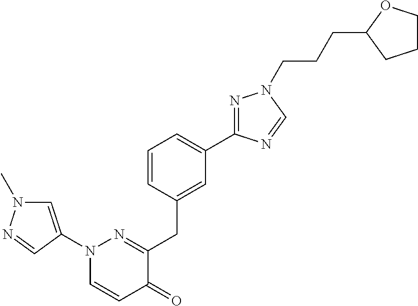 | rac-1-(1- methyl-1H- pyrazol-4-yl)-3- (3-{1-[3- (tetrahydrofuran- 2-yl)propyl]- 1H-1,2,4- triazol-3- yl}benzyl)pyridazin- 4(1H)-one | Calc'd 446, found 446 |

The syntheses of the alkyl halides (Intermediates #181 and 188-190) used in the synthesis of Examples #711-712 and 717-718 are shown below.

### Scheme 26

### Example #722

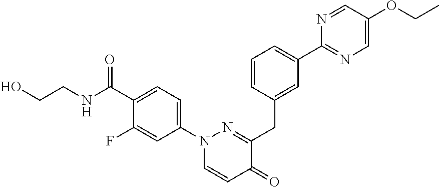

### 4-{3-[3-(5-Ethoxypyrimidin-2-yl)benzyl]-4-oxopyridazin-1(4H)-yl}-2-fluoro-N-(2-hydroxyethyl)benzamide

### Step 1. 4-{3-[3-(5-Ethoxypyrimidin-2-yl)benzyl]-4-oxopyridazin-1(4H)-yl}-2-fluoro-N-(2-hydroxyethyl)benzamide

1-(4-Bromo-3-fluorophenyl)-3-[3-(5-ethoxypyrimidin-2-yl)benzyl]pyridazin-4(1H)-one (Example #145, 50 mg, 0.10 mmol) was taken up in 1,4-dioxane (1.7 mL) in a microwave vial, and the reaction mixture was degassed by bubbling through argon. To this was added ethanolamine (6 μL, 0.10 mmol), molybdenum hexacarbonyl (27.4 mg, 0.104 mmol), DBU (62 μL, 0.42 mmol), tri-tert-butylphosphonium tetrafluoroborate (3.0 mg, 10μmol) and Hermann's catalyst (4.8 mg, 5.2 mmol). The reaction vial was capped and heated to 140° C. for 12 minutes under microwave irradiation (Biotage, Initiator). The reaction mixture was filtered through Celite, the solvent was removed in vacuo and the residue purified by reverse phase preparative HPLC to provide 4-{3-[3-(5-ethoxypyrimidin-2-yl)benzyl]-4-oxopyridazin-1(4H)-yl}-2-fluoro-N-(2-hydroxyethyl)benzamide.

LRMS (ESI) calc'd for C26H25FN5O4 [M+H]⁺: 490. Found: 490.

The following examples were prepared according to Scheme-26 following a similar procedure described for Example #722, which can be achieved by those of ordinary skill in the art of organic synthesis.

| | | IUPAC | Exact Mass |
|---|---|---|---|
| Example | Structure | Name | [M + H]⁺ |
| 723 | 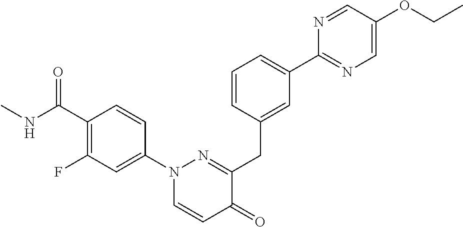 | 4-{3-[3-(5- ethoxypyrimidin-2- yl)benzyl-4- oxopyridazin- l(4H)-yl}-2- fluoro-N- methylbenzamide | Calc'd 460, found 460 |
| 724 | 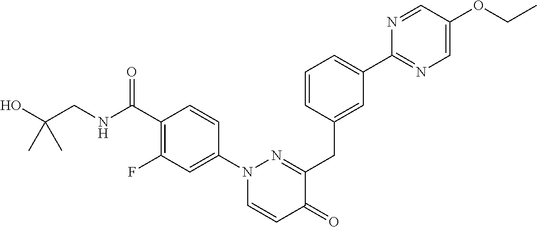 | 4-{3-[3-(5- ethoxypyrimidin-2- yl)benzyl]-4- oxopyridazin- fluoro-N-(2- 1(4H)-yl}-2- hydroxy-2- methylpropyl) benzamide | Calc'd 518, found 518 |

### Scheme 26

### Example #725

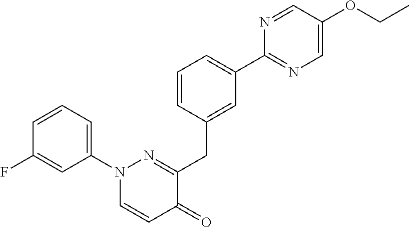

### 3-(3-(5-Ethoxypyrimidin-2-yl)benzyl)-1-(3-fluorophenyl)pyridazin-4(1H)-one

### Step 1. 3-[3-(5-Ethoxypyrimidin-2-yl)benzyl]-1-(3-fluorophenyl)pyridazin-4(1H)-one

1-(4-Bromo-3-fluorophenyl)-3-[3-(5-ethoxlpyrimidin-2-yl)benzyl]pyridazin-4(1H)-one (Example #145, 32.1 mg, 0.067 mmol) was dissolved in 1,4-dioxane (600 μL) in a microwave vial and the reaction solution was degassed by streaming argon through it for five minutes. To this was added N-methylethanolamine (0.014 mL, 0.173 mmol), molybdenum hexacarbonyl (18 mg, 0.067 mmol), DBU (40 μL, 0.26 mmol), tri-tert-butylphosphonium tetrafluoroborate (1 mg, 6 μmol), and Hermann's catalyst (3 mg, 3 μmol). The reaction vial was capped and heated to 140° C. for 40 minutes under microwave irradiation (Biotage, Initiator). The reaction was filtered through Celite and the volatiles removed under reduced pressure. The residue was purified by reverse phase preparative HPLC to provide 3-(3-(5-ethoxypyrimidin-2-yl)benzyl)-1-(3-fluorophenyl)pyridazin-4(1H)-one.

LRMS (ESI) calc'd for C23H20FN4O2 [M+H]⁺: 403. Found: 403.

### Scheme 26

### Example #726

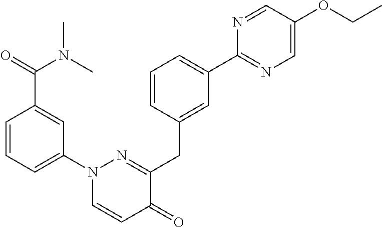

### 3-{3-[3-(5-Ethoxypyrimidin-2-yl)benzyl]-4-oxopyridazin-1(4H)-yl}-N,N-dimethylbenzamide

### Step 1. 3-{3-[3-(5-Ethoxypyrimidin-2-yl)benzyl]-4-oxopyridazin-1(4H)-yl}-N,N-dimethylbenzamide

A 10 mL round bottom flask was charged with 1-(3-bromophenyl)-3-[3-(5-ethoxypyrimidin-2-yl)benzyl]pyridazin-4(1H)-one (Example #143, 86 mg, 0.186 mmol), palladium (II) acetate (2.1 mg, 9.4 mol), DPPP (4 mg, 10 μmol) and DMF (1 mL). The atmosphere was purged under vacuum, backfilling with carbon monoxide gas (3×). Dimethylamine (40 wt % in THF, 0.20 mL, 1.58 mmol) was added and the reaction mixture was stirred at 85° C. for 18.5 hours under CO (1 atm). After cooling to room temperature, the reaction mixture was dissolved in 1 mL DMSO and purified by mass-triggered reverse-phase preparative HPLC to obtain 3-{3-[3-(5-ethoxypyrimidin-2-yl)benzyl]-4-oxopyridazin-1(4H)-yl}-N,N-dimethylbenzamide.

LRMS (ESI) calc'd for C26H26N5O3 [M+H]⁺: 456. Found: 456.

### Scheme 26

### Example #727

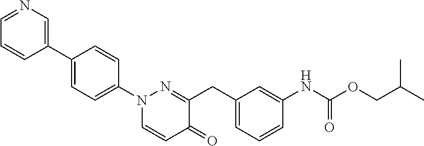

### 2-Methylpropyl[3-({4-oxo-1-[4-(pyridin-3-yl)phenyl]-1,4-dihydropyridazin-3-yl}methyl)phenyl]carbamate

### Step 1. 2-Methylpropyl[3-({4-oxo-1-[4-(pyridin-3-yl)phenyl]-1,4-dihydropyridazin-3-yl}methyl)phenyl]carbamate

A microwave vial was loaded with 2-methylpropyl (3-{[1-(4-bromophenyl)-4-oxo-1,4-dihydropyridazin-3-yl]methyl}phenyl)carbamate (Example #68, 25 mg, 0.055 mmol), Na₂CO₃ (18 mg, 0.17 mmol), Pd(PPh₃)₄ (3.0 mg, 2.7 μmol), pyridin-3-ylboronic acid (8.0 mg, 0.066 mmol) and suspended in DME (0.27 mL) and water (0.27 mL). The reaction mixture was heated in the microwave (Biotage, Initiator) for 100° C. for 10 minutes. The crude mixture was filtered through Celite, concentrated in vacuo, and purified by reverse phase preparative HPLC. Fractions containing the pure compound were collected and the free base was liberated using PL-HCO₃ cartridges (Stratospheres™, 0.9 mmol): The filtrate was frozen and freeze dried to afford 2-methylpropyl[3-({4-oxo-1-[4-(pyridin-3-yl)phenyl]-1,4-dihydropyridazin-3-yl}methyl)phenyl]carbamate.

LRMS (ESI) calc'd for C27H27N4O3 [M+H]⁺: 455. Found: 455.

The following examples were prepared from Examples #68-69 according to Scheme 26 following similar procedures described for Example #727, which can be achieved by those of ordinary skill in the art of organic synthesis.

| | | | Exact |
|---|---|---|---|
| | | | Mass |
| Example | Structure | IUPAC Name | [M + H]⁺ |
| 728 | 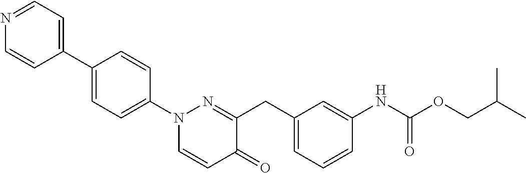 | 2-methylpropyl (3-({4-oxo-1- (4-(pyridin-4- yl)phenyl)-1,4- dihydropyridazin-3- yl}methyl}phenyl) carbamate | Calc'd 455, found 455 |
| 729 | 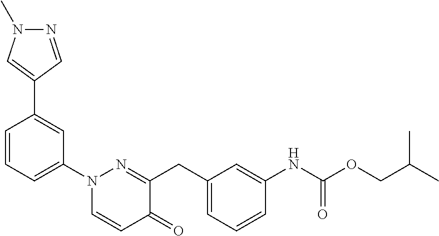 | 2-methylpropyl [3-({1-[3-(1- methyl-1H- pyrazol-4- yl)phenyl]-4- oxo-1,4- dihydropyridazin-3- yl}methyl)phenyl] carbamate | Calc'd 458, found 458 |
| 730 | 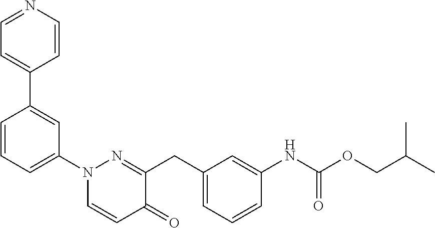 | 2-methylpropyl (3-{[4-oxo-1- (3-pyridin-4- ylphenyl)-1,4- dihydropyridazin-3- yl]methyl}phenyl) carbarnate | Calc'd 455, found 455 |
| 731 | 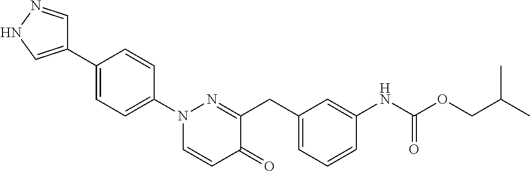 | 2-methylpropyl [3-({4-oxo-1- [4-(1H-pyrazol- 4-yl)phenyl]- 1,4- dihydropyridazin-3- yl}methyl)phenyl] carbamate | Calc'd 444, found 444 |
| 732 | 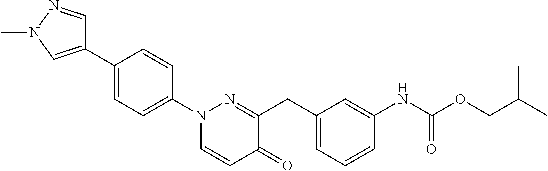 | 2-methylpropyl [3-({1-[4-(1- methyl-1H- pyrazol-4- yl)phenyl]-4- oxo-1,4- dihydropyridazin-3- yl}methyl)phenyl] carbamate | Calc'd 458, found 458 |

### Scheme 26

### Example #733

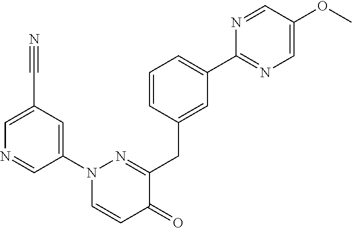

### 5-{3-[3-(5-Methoxypyrimidin-2-yl)benzyl]-4-oxopyridazin-1(4h)-yl}pyridine-3-carbonitrile

### Step 1. 5-{3-[3-(5-Methoxypyrimidin-2-yl)benzyl]-4-oxopyridazin-1(4H)-yl}pyridine-3-carbonitrile

In an oven-dried, N₂ cooled 2 mL microwave vial was placed Pd(PPh₃)₄ (2.0 mg, 1.6 μmol) and zinc cyanide (5.0 mg, 0.039 mmol), followed by a solution of 1-(5-bromopyridin-3-yl)-3-[3-(5-methoxypyrimidin-2-yl)benzyl]pyridazin-4(1H)-one (Intermediate #131, 15 mg, 0.032 mmol) in DMF (1 mL). The reaction mixture was degassed with a stream of N₂, then sealed and heated to 120° C. for 60 min under microwave irradiation (Biotage, Initiator). The reaction mixture was filtered through Celite, eluted with EtOAc, and concentrated in vacuo. The crude residue was purified by reverse phase preparative HPLC (10-100% MeCN—H₂O, 0.05% TFA). Fractions containing pure compound were collected and the free base was liberated using PL-HCO₃ cartridges (Stratospheres, 0.9 mmol). The filtrate was frozen and freeze dried to obtain 5-{3-[3-(5-methoxypyrimidin-2-yl)benzyl]-4-oxopyridazin-1(4H)-yl}pyridine-3-carbonitrile as a white solid.

LRMS (ESI) calc'd for C22H17N6O2 [M+H]⁺: 397. Found: 397.

### Scheme 27

### Example #734

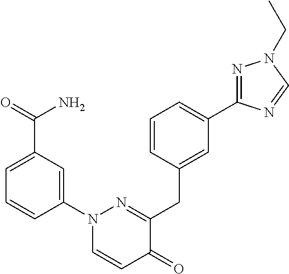

### 3-{3-[3-(1-Ethyl-1H-1,2,4-triazol-3-yl)benzyl]-4-oxopyridazin-1(4H)-yl}benzamide

### Step 1. 3-{3-[3-(1-Ethyl-1H-1,2,4-triazol-3-yl)benzyl]-4-oxopyridazin-4(4H)-yl}benzamide

3-{3-[3-(1-Ethyl-1H-1,2,4-triazol-3-yl)benzyl]-4-oxopyridazin-1(4H)-yl}benzonitrile (Example #84, 80 mg, 0.21 mmol) and K₂CO₃ (58 mg, 0.42 mmol) were taken up in DMSO (3 mL). Hydrogen peroxide 20 wt % (0.214 mL, 2.09 mmol) was added to the reaction flask. The mixture was allowed to stir at r.t. for 1 hour. Water (20 mL) was added and the flask was placed in an ice bath and allowed to stir for 30 minutes. The solid was filtered, rinsing the filter cake with cold water. Trituration with MeOH gave 3-{3-[3-(1-ethyl-1H-1,2,4-triazol-3-yl)benzyl]-4-oxopyridazin-1(4H)-yl}benzamide.

LRMS (ESI) calc'd for C22H21N₆O2 [M+H]⁺: 401. found 401.

The following examples were prepared from Examples #72, 73, 85, and 224 according to Scheme 27 following a similar procedure described for Example #734, which can be achieved by those of ordinary skill in the art of organic chemistry.

| | | | Exact |
|---|---|---|---|
| | | | Mass |
| Example | Structure | IUPAC Name | [M + H]⁺ |
| 735 | 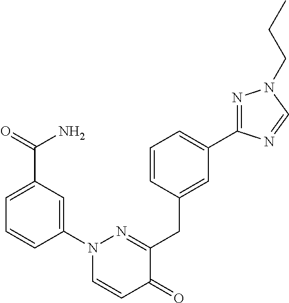 | 3-{4-oxo-3-[3-(1- propyl-1H-1,2,4- triazol-3- yl)benzyl]pyridazin- 1(4H)- yl}benzamide | Calc'd 415, found 415 |
| 736 | 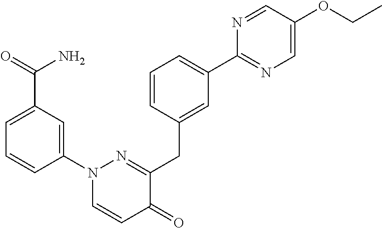 | 3-[3-[3-(5- ethoxypyrimidin- 2-yl)benzyl]-4- oxopyridazin- 1(4H)- yl]benzamide | Calc'd 428, found 428 |
| 737 | 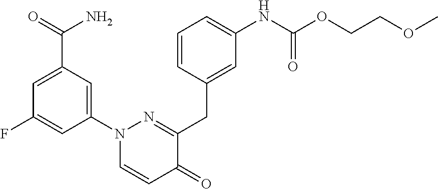 | 2-methoxyethyl [3-({1-[3- (aminocarbonyl)- 5-fluorophenyl]- 4-oxo-1,4- dihydropyridazin-3- yl}methyl)phenyl] carbamate | Calc'd 441, found 441 |
| 738 | 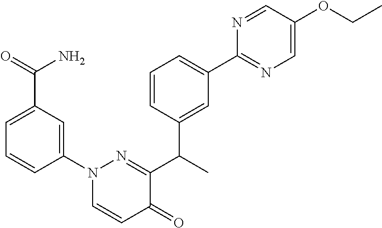 | rac-3-[3-{1-[3-(5- ethoxypyrimidin- 2- yl)phenyl]ethyl}- 4-oxopyridazin- 1(4H)- yl]lbenzamide | Calc'd 442, found 442 |

### Scheme 28

### Example #739

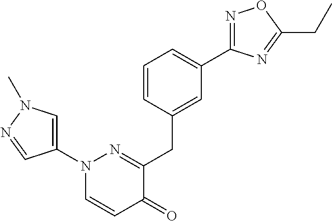

### 3-[3-(5-Ethyl-1,2,4-oxadiazole-3-yl)benzyl]-1-(1-methyl-1H-pyrazol-4-yl)pyridazin-4(1H)-one

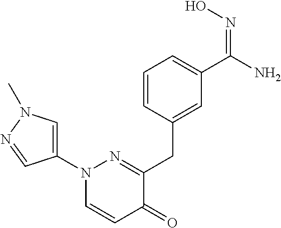

### Step 1. N-Hydroxy-3-{[1-(1-methyl-1H-pyrazol-4-yl)-4-oxo-1,4-dihydropyridazin-3-yl]methyl}benzenecarboximidamide

Hydroxylamine in water (50 wt %, 0.68 mL, 1 mmol) was added to a solution of 3-{[1-(1-methyl-1H-pyrazol-4-yl)-4-oxo-1,4-dihydropyridazin-3-yl]methyl}benzonitrile (Intermediate #129, 648 mg, 2.22 mmol) in EtOH (4 mL) and MeOH (8 mL) and the reaction was heated to 80° C. for 18 hours. The mixture was concentrated and the residue was washed with EtOAc and diethyl ether to give N-hydroxy-3-{[1-(1-methyl-1H-pyrazol-4-yl)-4-oxo-1,4-dihydropyridazin-3-yl]methyl}benzenecarboximidamide as a white solid.

LRMS (ESI) calc'd for C16H17N6O2 [M+H]⁺: 325. Found: 325.

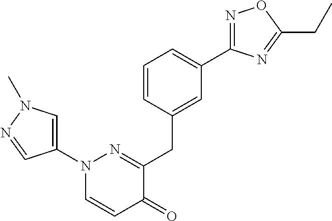

### Step 2. 3-[3-(5-Ethyl-1,2,4-oxadiazole-3-yl)benzyl]-1-(1-methyl-1H-pyrazol-4-yl)pyridazin-4(1H)-one

N-Hydroxy-3-{[1-(1-methyl-1H-pyrazol-4-yl)-4-oxo-1,4-dihydropyridazin-3-yl]methyl}benzenecarboximidamide (100 mg, 0.308 mmol), propionic acid (0.115 mL, 1.54 mmol), and EDC (148 mg, 0.771 mmol) were combined in DMF (3 mL). The mixture was allowed to stir at 50° C. for 3 hours. The reaction temperature was then increased to 85° C. and the reaction mixture was allowed to stir overnight. The reaction was filtered, concentrated in vacuo and purified by reverse phase preparative HPLC (20-70% MeCN—H₂O, 0.05% TFA), The fractions were collected, washed with saturated NaHCO₃, and the solvent was evaporated in vacuo to afford 3-[3-(5-ethyl-1,2,4-oxadiazole-3-yl)benzyl]-1-(1-methyl-1H-pyrazol-4-yl)pyridazin-4(1H)-one.

LRMS (ESI) calc'd for C19H19N6O2 [M+H]⁺: 363. Found: 363.

The following examples were prepared according to Scheme 28 following similar procedures described for Example #739, which can be achieved by those of ordinary skill in the art of organic synthesis.

| | | | Exact |
|---|---|---|---|
| | | | Mass |
| Example | Structure | IUPAC Name | [M + H]⁺ |
| 740 | 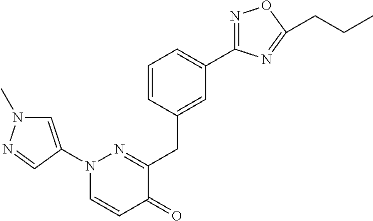 | 1-(1-methyl- 1H-pyrazol-4- yl)-3-[3-(5- propyl-1,2,4- oxadiazol-3- yl)benzyl]pyridazin- 4(1H)-one | Calc'd 377, found 377 |
| 741 | 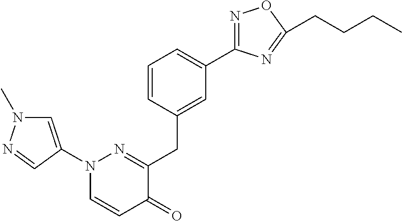 | 3-[3-(5-butyl- 1,2,4-oxadiazol- 3-yl)benzyl]-1- (1-methyl-1H- pyrazol-4- yl)pyridazin 4(1H)-one | Calc'd 391, found 391 |
| 742 | 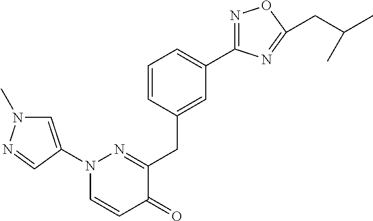 | 3-{3-[5-(2- methylpropyl)- 1,2,4-oxadiazol- 3-yl]benzyl}-1- (1-methyl-1H- pyrazol-4- yl)pyridazin- 4(1H)-one | Calc'd 391, found 391 |
| 743 | 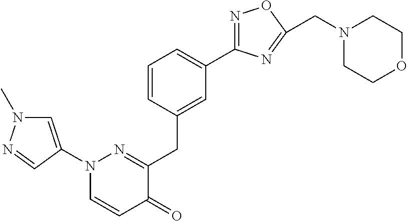 | 1-(1-methyl- 1H-pyrazol-4- yl)-3{3-[5- (morpholin-4- ylmethyl)-1,2,4- oxadiazol-3- yl]benzyl}pyridazin- 4(1H)-one | Calc'd 433, found 433 |
| 744 | 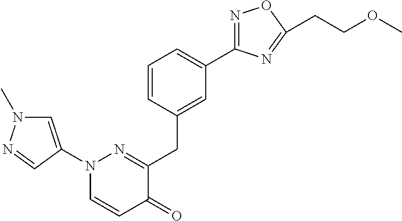 | 3-{3-[5-(2- methoxyethyl)- 1,2,4-oxadiazol- 3-yl]benzyl}-1- (1-methyl-1H- pyrazol-4- yl)pyridazin- 4(1H)-one | Calc'd 393, found 393 |
| 745 | 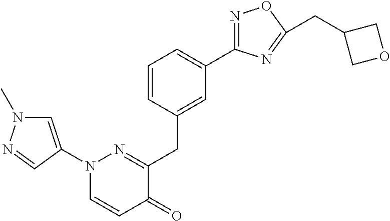 | 1-(1-methyl- 1H-pyrazol-4- yl)-3-{3-[5- (oxetan-3- ylmethyl)-1,2,4- oxadiazol-3- yl]benzyl}pyridazin- 4(1H)-one | Calc'd 405, found 405 |
| 746 | 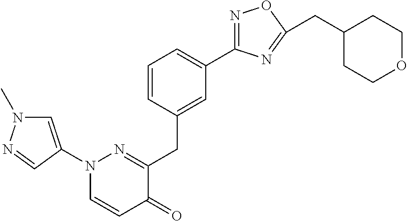 | 1-(1-methyl- 1H-pyrazol-4- yl-3-{3-[5- (tetrahydro-2H- pyran-4- ylmethyl)-1,2,4- oxadiazol-3- yl]benzyl}pyridazin- 4(1H)-one | Calc'd 433, found 433 |

### Scheme 29

### Examples #747 and #748

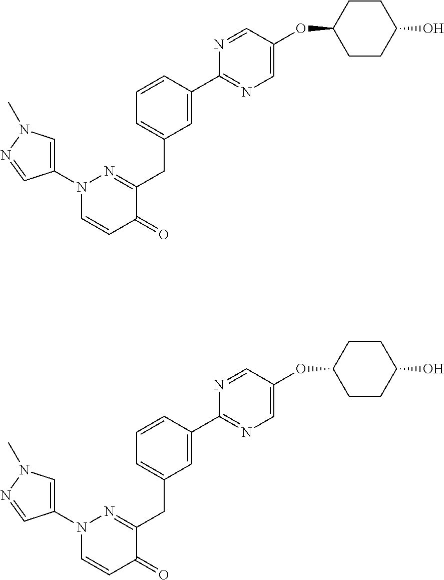

### 3-(3-{5-[(trans-4-Hydroxycyclohexyl)oxy]pyrimidin-2-yl}benzyl)-1-(1-methyl-1H-pyrazol-4-yl)pyridazin-4(1H)-one and 3-(3-{5-[(cis-4-hydroxycyclohexyl)oxy]pyrimidin-2-yl}benzyl)-1-(1-methyl-1H-pyrazol-4-yl)pyridazin-4(1H)-one

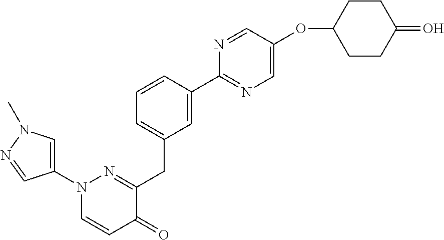

### Step 1. 1-(1-Methyl-1H-pyrazol-4-yl)-3-(3-{5-[4-oxocyclohexyl)oxy]pyrimidin-2-yl}benzyl)pyridazin-4(1H)-one

1-(1-Methyl-1H-pyrazol-4-yl)-3-(3-{5-[(4-oxocyclohexyl)oxy]pyrimidin-2-yl}benzyl)pyridazin-4(1H)-one was prepared from 3-{3-[5-(1,4-dioxaspiro[4.5]dec-8-yloxy)pyrimidin-2-yl]benzyl}-1-(1-methyl-1H-pyrazol-4-yl)pyridazin-4(1H)-one (Example #123) using the same procedure described for 4-{[2-(3-chlorophenyl)pyrimidin-5-yl]oxy}cyclohexanone (Intermediate #98 Step 1).

LRMS (ESI) calc'd for C25H25N6O3 [M+H]⁺: 457. Found: 457.

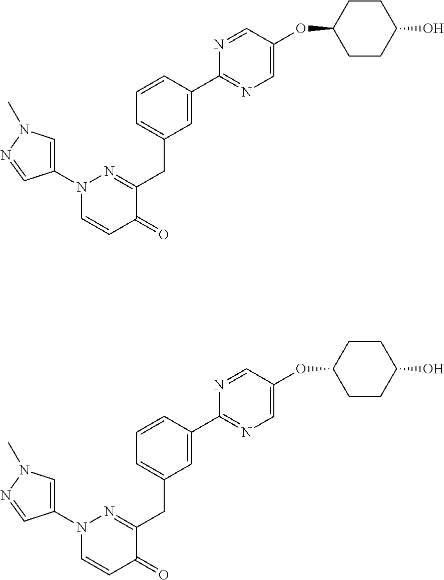

### Step 2. 3-(3-{5-[(trans-4-Hydroxycyclohexyl)oxy]pyrimidin-2-yl}benzyl)-1-(1-methyl-1H-pyrazol-4-yl)pyridazin-4(1H)-one and 3-(3-{5-[(cis-4-hydroxycyclohexyl)oxy]pyrimidin-2-yl}benzyl)-1-(1-methyl-1H-pyrazol-4-yl)pyridazin-4(1H)-one

1-(1-Methyl-1H-pyrazol-4-yl)-3-(3-{5-[(4-oxocyclohexyl)oxy]pyrimidin-2-yl}benzyl)pyridazin-4(1H)-one (190 mg, 0.42 mmol) was dissolved in methanol (3 mL) and cooled to 0° C. Sodium borohydride (23.6 mg, 0.62 mmol) was added. The reaction mixture was warmed to r.t. and stirred for 30 min. The reaction mixture was diluted with EtOAc and washed with brine. The organic layer was dried over Na₂SO₄, filtered, concentrated in vacuo and purified by reverse phase preparative HPLC (45-60% MeOH—H₂O, 0.1% TFA). The fractions containing the separated diastereoisomers were neutralized with saturated NaHCO₃, extracted into EtOAc and the solvent removed in vacuo to afford 3-(3-{5-[(trans-4-hydroxycyclohexyl)oxy]pyrimidin-2-yl}benzyl)-1-(1-methyl-1H-pyrazol-4-yl)pyridazin-4(1H)-one (Example #747) and 3-(3-{5-[(cis-4-hydroxycyclohexyl)oxy]pyrimidin-2-yl}benzyl)-1-(1-methyl-1H-pyrazol-4-yl)pyridazin-4(1H)-one (Example #748) as yellow solids.

### Example #747

LRMS (ESI) calc'd for C25H27N6O3 [M+H]⁺: 459. Found: 459.

### Example #748

LRMS (ESI) calc'd for C25H27N6O3 [M+H]⁺: 459. Found: 459.

### Scheme 30

### Example #749

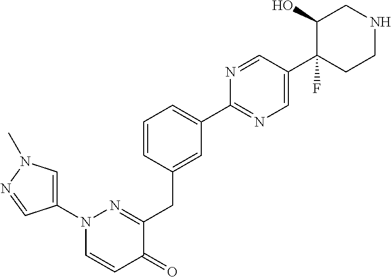

### rac-3-({3-[5-(trans-4-Fluoro-3-hydroxypiperidin-4-yl)pyrimidin-2-yl]benzyl}-1-(1-methyl-1H-pyrazol-4-yl)pyridazin-4(1H)-one

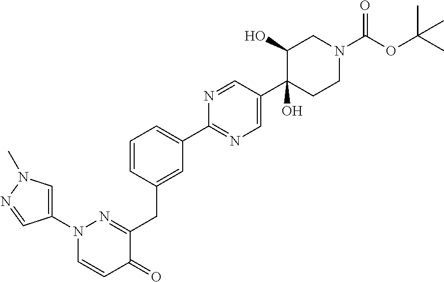

### Step 1. rac-tert-Butyl cis-3,4-dihydroxy-4-[2-(3-{[1-(1-methyl-1H-pyrazol-4-yl)-4-oxo-1,4-dihydropyridazin-3-yl]methyl}phenyl)pyrimidin-5-yl]piperidine-1-carboxylate

To a biphasic mixture of tert-butyl 4-[2-(3-{[1-(1-methyl-1H-pyrazol-4-yl)-4-oxo-1,4-dihydropyridazin-3-yl]methyl}phenyl)pyrimidin-5-yl]-3,6-dihydropyridine-1(2H)-carboxylate (Intermediate #118, 50 mg, 0.095 mmol) in THF (1 mL) and water (0.5 mL) was added NMO (14 mg, 0.11 mmol) and osmium tetroxide (4% solution in water, 0.030 mL, 4.8 μmol). The reaction mixture was stirred (wrapped in foil) at room temperature for a total of 5 days. The reaction was quenched with 1 mL of 10% aqueous sodium thiosulfate and diluted with EtOAc before filtering through Celite. Silica gel was added to the filtrate and the resulting mixture was concentrated to a crude solid that was purified by flash chromatography (MPLC, 1-10% MeOH-EtOAc) to afford a colorless oil. The oil was taken up into DCM, hexanes was added and the resulting mixture was concentrated to afford rac-tert-butyl cis-3,4-dihydroxy-4-[2-(3-{[1-(1-methyl-1H-pyrazol-4-yl)-4-oxo-1,4-dihydropyridazin-3-yl]methyl}phenyl)pyrimidin-5-yl]piperidine-1-carboxylate as a white foam.

LRMS (ESI) calc'd for C29H34N7O5 [M+H]⁺, 560. found 560.

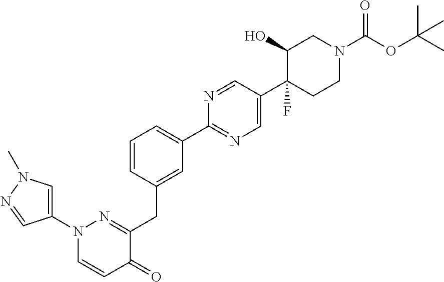

### Step 2. rac-tert-Butyl trans-4-fluoro-3-hydroxy-4-[2-(3-{[1-(1-methyl-1H-pyrazol-4-yl)-4-oxo-1,4-dihydropyridazin-3-yl]methyl}phenyl)pyrimidin-5-yl]piperidine-1-carboxylate

To a stirred solution of rac-tert-Butyl cis-3,4-dihydroxy-4-[2-(3-{[1-(1-methyl-1H-pyrazol-4-yl)-4-oxo-1,4-dihydropyridazin-3-yl]methyl}phenyl)pyrimidin-5-yl]piperidine-1-carboxylate (44 mg, 0.079 mmol) in DCM (1 mL) at 0° C. was added DAST (0.030 mL, 0.17 mmol). The resulting mixture was stirred in the ice bath (as it naturally warmed) for 90 min, then quenched with aqueous sodium bicarbonate. The resulting biphasic mixture was diluted with EtOAc and filtered through Celite. The filtrate was concentrated to a purple residue that was purified by reverse phase preparative HPLC (35-70% MeCN-water, 0.1% TFA) to afford rac-tert-butyl trans-4-fluoro-3-hydroxy-4-[2-(3-{[1-(1-methyl-1H-pyrazol-4-yl)-4-oxo-1,4-dihydropyridazin-3-yl]methyl}phenyl)pyrimidin-5-yl]piperidine-1-carboxylate.

LRMS (ESI) calc'd for C29H33FN7O4 [M+H]⁺, 562. found 562.

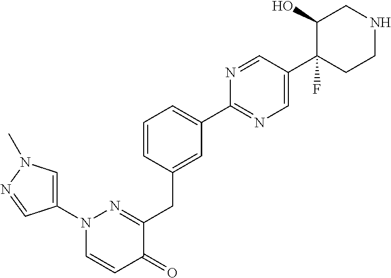

### Step 3. rac-3-{3-[5-(trans-4-Fluoro-3-hydroxypiperidin-4-yl)pyrimidin-2-yl]benzyl}-1-(1-methyl-1H-pyrazol-4-yl)pyridazin-4(1H)-one

To a stirred solution of rac-tert-butyl trans-4-fluoro-3-hydroxy-4-[2-(3-{[1-(1-methyl-1H-pyrazol-4-yl)-4-oxo-1,4-dihydropyridazin-3-yl]methyl}phenyl)pyrimidin-5-yl]piperidine-1-carboxylate (12 mg, 0.021 mmol) in methanol (1 mL) was added 4N HCl in 1,4-dioxane (1 mL). The resulting solution was stirred at r.t. for 1 hour. The reaction mixture was concentrated to a residue that was dissolved in MeCN and water and lyophilized to afford the HCl salt of rac-3-{3-[5-(trans-4-fluoro-3-hydroxypiperidin-4-yl)pyrimidin-2-yl]benzyl}-1-(1-methyl-1H-pyrazol-4-yl)pyridazin-4(1H)-one as a light yellow fluffy solid.

LRMS (ESI) calc'd for C24H25FN7O2 [M+H]⁺, 462. found 462.

### Scheme 31

### Example #750

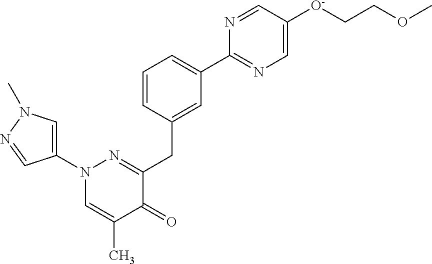

### 3-{3-[5-(2-Methoxyethoxy)pyrimidin-2-yl]benzyl}-5-methyl-1-(1-methyl-1H-pyrazol-4-yl)pyridazin-4(1H)-one

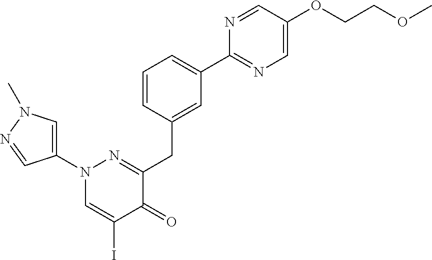

### Step 1. 5-Iodo-3-{3-[5-(2-methoxyethoxy)pyrimidin-2-yl]benzyl}-1-(1-methyl-1H-pyrazol-4-yl)pyridazin-4(1H)-one

3-{3-[5-(2-Methoxyethoxy)pyrimidin-2-yl]benzyl}-1-(1-methyl-1H-pyrazol-4-yl)pyridazin-4(1H)-one (Example #4, 150 mg, 0.358 mmol) was taken up in DCM (1.2 mL). Bis(pyridine)iodonium tetrafluoroborate (147 mg, 0.394-mmol) was added, followed by triflic acid (0.070 mL, 0.789 mmol) and the resulting mixture stirred at room temperature for 5 hours. Saturated NaHCO₃ was added and the products extracted into DCM (x2). The combined organic extracts were washed with 2 N HCl and brine, dried over Na₂SO₄ and concentrated in vacuo. Purification of the residue by flash chromatography (MPLC, 40-100% EtOAc-hexanes followed by 0-10% MeOH-EtOAc) gave 5-iodo-3-{3-[5-(2-methoxyethoxy)pyrimidin-2-yl]benzyl}-1-(1-methyl-1H-pyrazol-4-yl)pyridazin-4(1H)-one as a pale yellow solid.

LRMS (ESI) calc'd for C22H22IN6O3 [M+H]⁺: 545. Found: 545.

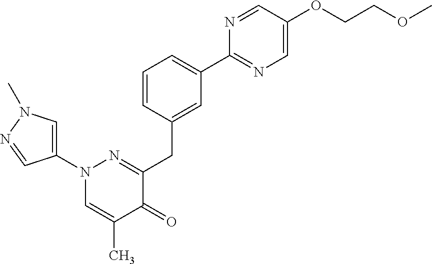

### Step 2. 3-{3-[5-(2-Methoxyethoxy)pyrimidin-2-yl]benzyl}-5-methyl-1-(1-methyl-1H-pyrazol-4-yl)pyridazin-4(1H)-one

An oven-dried, nitrogen cooled 2 mL microwave vial was charged with 5-iodo-3-{3-[5-(2-methoxyethoxy)pyrimidin-2-yl]benzyl}-1-(1-methyl-1H-pyrazol-4-yl)pyridazin-4(1H)-one (50 mg, 0.092 mmol), PdCl₂(dppf).DCM adduct (6.8 mg, 8.3 μmol), Cs₂CO₃ (0.09 g, 0.3 mmol) and potassium methyltrifluoroborate (16.8 mg, 0.138 mmol), sealed under a nitrogen atmosphere and charged with THF (0.4 mL) and degassed water (0.02 mL). The reaction mixture was heated to 100° C. for 110 hours then cooled to room temperature, diluted with ethyl acetate and filtered through Celite eluting with ethyl acetate. The filtrate was concentrated in vacuo and purified by flash chromatography (MPLC, 0-20% MeOH/DCM) to obtain 3-{3-[5-(2-methoxyethoxy)pyrimidin-2-yl]benzyl}-5-methyl-1-(1-methyl-1H-pyrazol-4-yl)pyridazin-4(1H)-one.

LRMS (ESI) calc'd for C23H25N6O3 [M+H]⁺: 433. Found: 433,

### Scheme 32

### Example #751

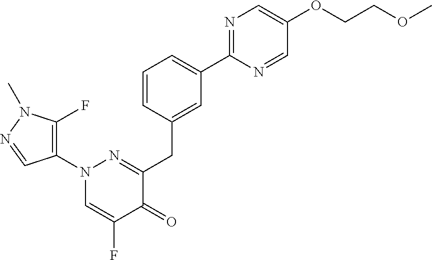

### 5-Fluoro-1-(5-fluoro-1-methyl-1H-pyrazol-4-yl)-3-{3-[5-(2-methoxyethoxy)pyrimidin-2-yl]benzyl}pyridazin-4(1h)-one

### Step 1. 5-Fluoro-1-(5-fluoro-1-methyl-1H-pyrazol-4-yl)-3-{3-[5-(2-methoxyethoxy)pyrimidin-2-yl]benzyl}pyridazin-4(1H)-one

To a microwave vial equipped with a stir bar was added 3-{3-[5-(2-methoxyethoxy)pyrimidin-2-yl]benzyl}-1-(1-methyl-1H-pyrazol-4-yl)pyridazin-4(1H)-one (Example #4, 40.0 mg, 0.096 mmol), Selectfluor (119 mg, 0.335 mmol), and MeCN (1900 μL). The reaction mixture was heated to 150° C. for 10 minutes under microwave-irradiation (Biotage, initiator). The crude reaction mixture was concentrated in vacuo. The residue was purified by flash chromatography (MPLC, 0-100% EtOAc-hexanes followed by 0-15% MeOH-DCM) to give 5-fluoro-1-(5-fluoro-1-methyl-1H-pyrazol-4-yl)-3-{3-[5-(2-methoxyethoxy)pyrimidin-2-yl]benzyl}pyridazin-4(1H)-one.

LRMS (ESI) calc'd for C22H21F2N6O3 [M+H]⁺: 455. Found: 455.

The following example was prepared from Example #3 according to Scheme 32 following a similar procedure described for Example #751, which can be achieved by those of ordinary skill in the art of organic synthesis.

| | | | Exact |
|---|---|---|---|
| | | IUPAC | Mass |
| Example | Structure | Name | [M + H]⁺ |
| 752 | 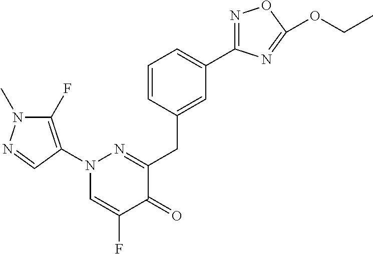 | 3-[3-(5- ethoxypyrimidin- 2- yl)benzyl-5- fluoro-1-(5- fluoro-1- methyl-1H- pyrazol-4- yl)pyridazin- 4(1H)-one | Calc'd 425, found 425 |

### Scheme 33

### Example #753

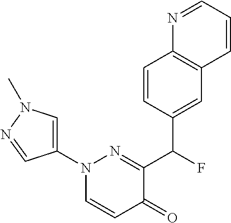

### rac-3-[Fluoro(quinolin-6-yl)methyl]-1-(1-methyl-1H-pyrazol-4-yl)pyridazin-4(1H)-one

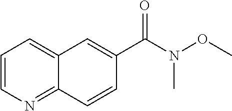

### Step 1. N-Methoxy-N-methylquinoline-6-carboxamide

Quinoline-6-carboxylic acid (3.4 g, 19.6 mmol), N,O-dimethylhydroxylamine hydrochloride (3.83 g, 39.3 mmol), DIPEA (13.7 mL, 79.0 mmol), EDC (5.65 g, 29.5 mmol) and HOBT (4.51 g, 29.5 mmol) were stirred in THF (100 mL) at room temperature for 18 hours. Saturated NaHCO₃ was added and the products extracted into EtOAc (x2). The combined organic extracts were washed with brine, dried over MgSO₄ and concentrated in vacuo to give N-methoxy-N-methylquinoline-6-carboxamide as a yellow gum.

LRMS (ESI) calc'd for C12H13N2O2 [M+H]⁺: 217. Found: 217.

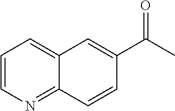

### Step 2. 1-Quinolin-6-ylethanone

N-Methoxy-N-methylquinoline-6-carboxamide (3.95 g, 18.3 mmol) was taken up in THF (220 mL) and cooled to −78° C. Methylmagnesium bromide (3 M in Et₂O, 18.27 mL, 54.8 mmol) was added and the resulting mixture stirred at −78° C. for 10 minutes before warming to room temperature and stirring for 2 hours. TLC The mixture was cooled to −78° C. and 2 N HCl was added to quench. The mixture was neutralized with solid Na₂CO₃ and the products extracted into EtOAc (x3). The combined organic extracts were washed with brine, dried over MgSO₄ and concentrated in vacuo. Purification of the residue by flash chromatography (MPLC, 12-100% EtOAc-hexanes) gave 1-quinolin-6-ylethanone as a white solid.

LRMS (ESI) calc'd for C11H10NO [M+H]⁺: 172. Found: 172.

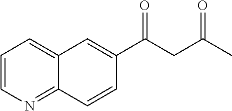

### Step 3. 1-Quinolin-6-ylbutane-1,3-dione

NaHMDS (1 M in THF, 4.14 mL, 4.14 mmol) was taken up in a 20 mL microwave vial and cooled to −78° C. A solution of 1-quinolin-6-ylethanone (308 mg, 1.80 mmol) in THF (5 mL) was added dropwise via syringe and the resulting mixture stirred at −78° C. for 15 minutes. Room temperature was attained, EtOAc (0.264 mL, 2.70 mmol) was added and the resulting mixture stirred at 70° C. for 18 hours. Saturated NH₄Cl was added and the products extracted into EtOAc (x2). The combined organic extracts were washed with brine, dried over Na₂SO₄ and concentrated in vacuo. Purification of the residue by flash chromatography (MPLC, 5-60% EtOAc-hexanes) gave 1-quinolin-6-ylbutane-1,3-dione as a beige solid.

LRMS (ESI) calc'd for C13H12NO2 [M+H]⁺: 214. Found: 214.

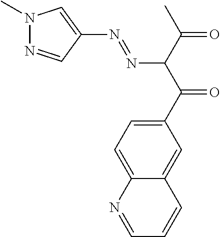

### Step 4. 2-[(E)-(1-Methyl-1H-pyrazol-4-yl)diazenyl]-1-quinolin-6-ylbutane-1,3-dione

1-Methyl-1H-pyrazol-4-amine hydrochloride (73 mg, 0.546 mmol) was dissolved in c. HCl (0.34 mL)/water (1.7 mL) and cooled to 0° C. A solution of sodium nitrite (39.6 mg, 0.574 mmol) in water (0.8 mL) was added dropwise while maintaining the internal temperature at <4° C. On complete addition, the mixture was stirred at 0° C. for 20 minutes. The resulting diazonium chloride solution was added via a pipette to a solution of 1-methyl-1H-pyrazol-4-amine hydrochloride (73 mg, 0.546 mmol) and NaOAc (672 mg, 8.20 mmol) in water (0.8 mL)/EtOH (0.8 mL) at 0° C. The mixture was stirred at 00° C. for 20 minutes. Saturated NaHCO₃ was added and the products extracted into EtOAc (x2) followed by MeOH-DCM. The combined organic extracts were concentrated in vacuo while loading onto Na₂SO₄. Purification of the residue by flash chromatography (MPLC, 0-5% MeOH-DCM) followed by re-purification of the product fractions by flash chromatography (MPLC, 12-100% EtOAc-hexanes followed by 0-10% MeOH-EtOAc) gave 2-[(E)-(1-methyl-1H-pyrazol-4-yl)diazenyl]-1-quinolin-6-ylbutane-1,3-dione as an orange solid.

LRMS (ESI) calc'd for C17H16N5O2 [M+H]⁺: 322. Found: 322.

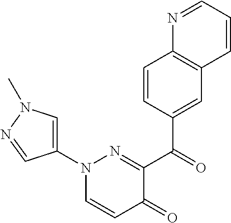

### Step 5. 1-(1-Methyl-1H-pyrazol-4-yl)-3-(quinolin-6-ylcarbonyl)pyridazin-4(1H)-one

2-[(E)-(1-Methyl-1H-pyrazol-4-yl)diazenyl]-1-quinolin-6-ylbutane-1,3-dione (103 mg, 0.321 mmol) was stirred in refluxing DMFDMA (3 mL) for 1 hour. Room temperature was attained and the solvent removed in vacuo. The residue was recrystallized from EtOH to give 1-(1-methyl-1H-pyrazol-4-yl)-3-(quinolin-6-ylcarbonyl)pyridazin-4(1H)-one as an orange solid.

LRMS (ESI) calc'd for C18H14N5O2 [M+H]⁺: 332. Found: 332.

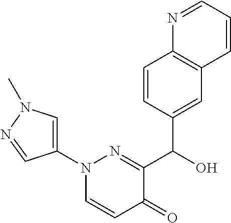

### Step 6. rac-3-[Hydroxy(quinolin-6-yl)methyl]-1-(1-methyl-1H-pyrazol-4-yl)pyridazin-4(1H)-one

rac-3-[Hydroxy(quinolin-6-yl)methyl]-1-(1-methyl-1H-pyrazol-4-yl)pyridazin-4(1H)-one was prepared from 1-(1-methyl-1H-pyrazol-4-yl)-3-(quinolin-6-ylcarbonyl)pyridazin-4(1H)-one according to the procedure described for rac-3-[[3-(5-ethoxypyrimidin-2-yl)phenyl](hydroxy)methyl]-1-(1-methyl-1H-pyrazol-4-yl)pyridazin-4(LB)-one (Example #654 Step 4).

LRMS (ESI) calc'd for C18H16N5O2 [M+H]⁺: 334. Found: 334.

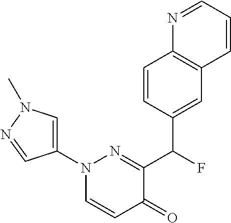

### Step 7. rac-3-[Fluoro(quinolin-6-yl)methyl]-1-(1-methyl-1H-pyrazol-4-yl)pyridazin-4(1H)-one

rac-3-[Fluoro(quinolin-6-yl)methyl]-1-(1-methyl-1H-pyrazol-4-yl)pyridazin-4(1H)-one was prepared from rac-3-[hydroxy(quinolin-6-yl)methyl]-1-(1-methyl-1H-pyrazol-4-yl)pyridazin-4(1H)-one according to the procedure described for rac-3-[[3-(5-ethoxypyrimidin-2-yl)phenyl](fluoro)methyl]-1-(1-methyl-1H-pyrazol-4-yl)pyridazin-4(1H)-one (Examples #655-656 Step 1).

LRMS (ESI) calc'd for C18H15FN5O [M+H]⁺: 336. Found: 336.

### Scheme 34

### Example #754

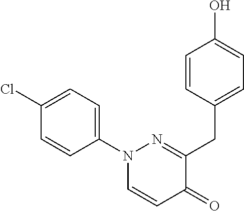

### 1-(4-Chlorophenyl)-3-(4-hydroxybenzyl)pyridazin-4(1H)-one

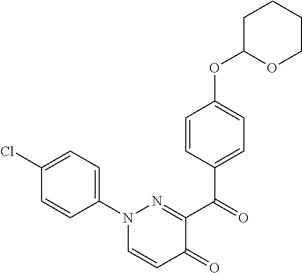

### Step 1. 1-(4-Chlorophenyl)-3-[4-(tetrahydro-2H-pyran-2-yloxy)benzoyl]pyridazin-4(1H)-one

A suspension of 1-(4-chlorophenyl)-N-methoxy-N-methyl-4-oxo-1,4-dihydropyridazine-3-carboxamide (Example #640 Step 1, 110 mg, 0.375 mmol) in THF (3.7 mL) was cooled to −78° C. and, 4-(2-tetrahydro-2H-pyranoxy)phenylmagnesium bromide (1.5 mL, 0.75 mmol) was added dropwise. The resulting mixture stirred at −78° C. for 2 hrs. Saturated NH₄Cl was added (at −78° C.) and the aqueous layer was extracted with EtOAc (2×). The combined organic extracts were washed with brine, dried over MgSO₄, and concentrated in vacuo. Purification of the residue by flash chromatography (MPLC, (40-100% EtOAc-Hexanes) gave 1-(4-chlorophenyl)-3-[4-(tetrahydro-2H-pyran-2-yloxy)benzoyl]pyridazin-4(1H)-one as a yellow gum.

LRMS (ESI) calc'd for C22H20N2O4 [M+H]⁺: 411. found: 411

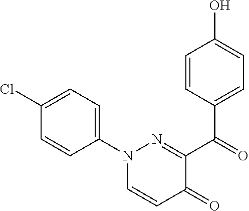

### Step 2. 1-(4-Chlorophenyl)-3-(4-hydroxybenzoyl)pyridazin-4(1H)-one

1-(4-Chlorophenyl)-3-[4-(tetrahydro-2H-pyran-2-yloxy)benzoyl]pyridazin-4(1H)-one (53 mg, 0.13 mmol) was taken up in DCM (5 mL) and 4 M HCl in 1,4-dioxane (0.08 mL, 0.3 mmol) was added dropwise via syringe. The resulting suspension was stirred at r.t. for 2 hours. The solvent was removed in vacuo and the residue was triturated in EtOAc to give 1-(4-chlorophenyl)-3-(4-hydroxybenzoyl)pyridazin-4(1H)-one as an off-white solid.

LRMS (ESI) calc'd for C17H12ClN2O3 [M+H]⁺: 327. found: 327.

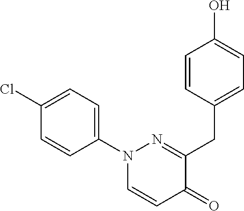

### Step 3. 1-(4-C-chlorophenyl)-3-(4-hydroxybenzyl)pyridazin-4(1H)-one

1-(4-Chlorophenyl)-3-(4-hydroxybenzoyl)pyridazin-4(1H)-one (19 mg, 0.058 mmol) was taken up in DCM (0.6 mL) and cooled to 0° C. HF.Pyr (60 L) was added, followed by triethylsilane (0.024 mL, 0.15 mmol) and the resulting mixture stirred at 0° C. for 10 minutes and r.t. for 3 hours. Additional HF.Pyr (60 μL) and triethylsilane (0.024 mL, 0.15 mmol) were added and stirring continued at r.t. for 3 hours. Additional HF.Pyr (60 μL) and triethylsilane (0.024 mL, 0.15 mmol) were added and stirring continued at r.t. overnight. TFA (0.5 mL) was added followed by additional HF.Pyr (60 μL) and triethylsilane (0.024 mL, 0.15 mmol) and the resulting mixture stirred at 60° C. for 4 hours. Additional triethylsilane (0.024 mL, 0.15 mmol) was added and stirring continued overnight. Additional triethylsilane (0.024 mL, 0.15 mmol) was added and stirring at 60° C. continued overnight. Additional triethylsilane (0.024 mL, 0.15 mmol) was added and stirring at 60° C. continued for 3 days. R.t. was attained, saturated NaHCO₃ was added and the products extracted into EtOAc (2×). The combined organic extracts were washed with 1 N HCl (2×) and brine, dried over MgSO₄ and concentrated in vacuo. Purification of the residue by reverse phase preparative HPLC (40-80% MeCN—H₂O, 0.05% TFA) afforded 1-(4-chlorophenyl)-3-(4-hydroxybenzyl)pyridazin-4(1H)-one as an off-white solid.

LRMS (ESI) calc'd for C17H14ClN2O2 [M+H]⁺: 313. found: 313.

### Scheme 35

### Example #755

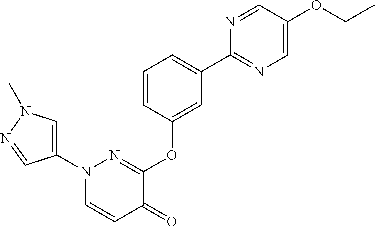

### 3-[3-(5-Ethoxypyrimidin-2-yl)phenoxy]-1-(1-methyl-1H-pyrazol-4-yl)pyridazin-4(1H)-one

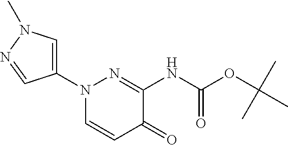

### Step 1. tert-Butyl [1-(1-methyl-1H-pyrazol-4-yl)-4-oxo-1,4-dihydropyridazin-3-yl]carbamate

In a 250 mL round bottom flask was placed 1-(1-methyl-1H-pyrazol-4-yl)-4-oxo-1,4-dihydropyridazine-3-carboxylic acid (Intermediate #1 Step 3, 5.0 g, 23 mmol), tert-BuOH (40 mL) and triethylamine (5.0 mL, 370 mmol). DPPA (6.0 mL, 28 mmol) was added dropwise and a reflux condenser was attached. The reaction mixture was heated to reflux for 17.5 hours. After cooling to room temperature, the reaction mixture was diluted with water and extracted with EtOAc (3×). The combined organic extracts were washed with saturated NaHCO₃ (3×), water-(2×) and brine, dried over MgSO₄, filtered and concentrated in vacuo. Purification of the residue by flash chromatography (MPLC, 0-20% MeOH/DCM) gave tert-butyl [1-(1-methyl-1H-pyrazol-4-yl)-4-oxo-1,4-dihydropyridazin-3-yl]carbamate.

LRMS (ESI) calc'd for C13H18N5O3 [M+H]⁺: 292. Found: 292.

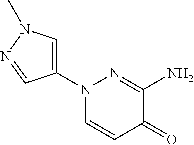

### Step 2. 3-Amine-1-(1-methyl-1H-pyrazol-4-yl)pyridazin-4(1H)-one

To a stirring solution of tert-butyl [1-(1-methyl-1H-pyrazol-4-yl)-4-oxo-1,4-dihydropyridazin-3-yl]carbamate (5.31 g, 18.2 mmol) in 1,4-dioxane (65 mL) was added 4N HCl in 1,4-dioxane (100 mL, 400 mmol). The reaction mixture was stirred for 67 hours at room temperature and then filtered to collect precipitate which was washed with ethyl acetate followed by hexanes, taken up in methanol, concentrated onto silica gel and purified by flash chromatography (MPLC, 0-20% MeOH/DCM) to obtain 3-amino-1-(1-methyl-1H-pyrazol-4-yl)pyridazin-4(1H)-one.

LRMS (ESI) calc'd for C8H10N50 [M+H]⁺: 192. Found: 192.

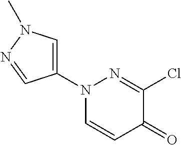

### Step 3. 3-Chloro-1-(1-methyl-1H-pyrazol-4-yl)pyridazin-4(1H)-one

To a suspension of 3-amino-1-(1-methyl-1H-pyrazol-4-yl)pyridazin-4(1H)-one (50 mg, 0.26 mmol) in concentrated HCl (2 mL) at 0° C. was added dropwise a solution of NaNO₂ (18.9 mg, 0.274 mmol) in water (0.2 mL). The reaction mixture was stirred at 0° C. for 110 min, then carefully quenched with 2N NaOH until basic. The products were extracted into DCM (x3), washed with brine, dried over MgSO₄, filtered and concentrated in vacuo to give 3-chloro-1-(1-methyl-1H-pyrazol-4-yl)pyridazin-4(1H)-one.

LRMS (ESI) calc'd for C8H8ClN4O [M+H]⁺: 211. Found: 211.

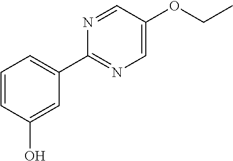

### Step 4. 3-(5-Ethoxypyrimidin-2-yl)phenol

To a stirring mixture of 5-ethoxy-2-[3-(4,4,5,5-tetramethyl-1,3,2-dioxaborolan-2-yl)phenyl]pyrimidine (Intermediate #86 Step 5, 0.15 g, 0.46 mmol) and 2 N NaOH (0.3 mL, 0.6 mmol) in THF (2 mL) was added 30% aqueous hydrogen peroxide (0.06 mL, 0.6 mmol). This mixture was stirred at room temperature for 70 min. The reaction mixture was quenched with saturated NH₄Cl, extracted with DCM (3×), washed with brine, dried over MgSO₄, filtered and concentrated in vacuo to give 3-(5-ethoxypyrimidin-2-yl)phenol.

LRMS (ESI) calc'd for C12H13N2O2 [M+H]⁺: 21-7. Found: 217.

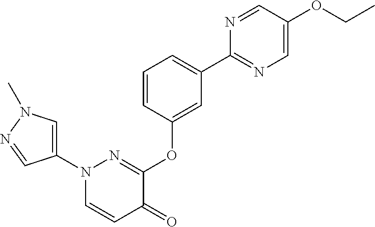

### Step 5. 3-[3-(5-Ethoxypyrimidin-2-yl)phenoxy]-1-(1-methyl-1H-pyrazol-4-yl)pyridazin-4(1H)-one

A 5 mL microwave vial was charged with 3-chloro-1-(1-methyl-1H-pyrazol-4-yl)pyridazin-4(1H)-one (50 mg, 0.237 mmol), potassium carbonate (0.10 g, 0.724 mmol), 3-(5-ethoxypyrimidin-2-yl)phenol (109.6 mg, 0.400 mmol) and DMF (2 mL) then sealed with a septum and heated to 100° C. for 15 hours. After cooling to room temperature, the reaction mixture was poured into water, filtered and dried via lyophilizer to obtain 3-[3-(5-ethoxypyrimidin-2-yl)phenoxy]-1-(1-methyl-1H-pyrazol-4-yl)pyridazin-4(1H)-one.

LRMS (ESI) calc'd for C20H19N6O3 [M+H]⁺: 391. Found: 391.

### Scheme 36

### Example #756

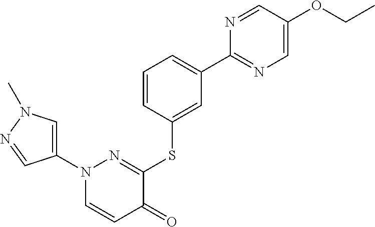

### 3-{[3-(5-Ethoxypyrimidin-2-yl)phenyl]sulfanyl}-1-(1-methyl-1H-pyrazol-4-yl)pyridazin-4(1H)-one

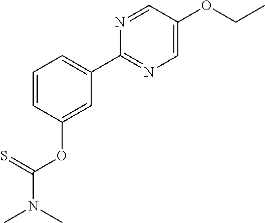

### Step 1. O-[3-(5-Ethoxypyrimidin-2-yl)phenyl]dimethylcarbamothioate

An oven-dried, nitrogen cooled 5 mL microwave vial was charged with 2-(3-chlorophenyl)-5-ethoxypyrimidine (121 mg, 0.513 mmol, Intermediate #86 Step 4), Pd₂(dba)₃ (12 mg, 0.013 mmol), Me₄^{t}BuXPhos (13 mg, 0.027 mmol) and freshly ground potassium hydroxide (90 mg, 1.6 mmol). The vial was sealed under a nitrogen atmosphere, 1,4-dioxane (1 mL) and degassed water (1 mL) were added and the reaction heated to 100° C. for 15 hours. After cooling to room temperature, N,N-dimethylthiocarbamoyl chloride (108 mg, 0.873 mmol) was added and the reaction mixture was stirred at room temperature for 6 hours. Additional N,N-dimethylthiocarbamoyl chloride (0.08 g, 0.6 mmol) was added and the reaction mixture was heated to 50° C. for 17 hours. After cooling to room temperature, the reaction mixture was filtered through Celite, eluting with EtOAc, concentrated in vacuo and purified by flash chromatography (MPLC, 25-100% EtOAc-hexanes) to obtain 0-[3-(5-ethoxypyrimidin-2-yl)phenyl]dimethylcarbamothioate.

LRMS (ESI) calc'd for C15H18N3O2S [M+H]⁺: 304. Found: 304.

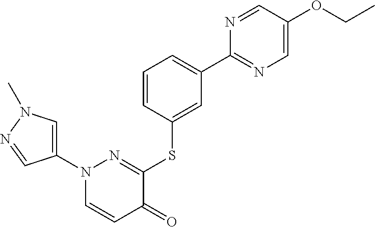

### Step 2. 3-{[3-(5-Ethoxypyrimidin-2-yl)phenyl]sulfanyl}-1-(1-methyl-1H-pyrazol-4-yl)pyridazin-4(1H)-one

A solution of O-[3-(5-ethoxypyrimidin-2-yl)phenyl]dimethylcarbamothioate (60.1 mg, 0.198 mmol) in N-methyl-2-pyrrolidinone (2 mL) was heated to 250° C. for 2 hours under microwave irradiation. Added 2N sodium hydroxide (0.3 mL, 0.6 mmol), then heated to 100° C. for 23 hours, then added more 2N sodium hydroxide (0.5 mL, 1 mmol) and heated to 100° C. for 5.5 hours. The reaction mixture was cooled to room temperature and allowed to stir at this temperature over the weekend. At this point, 3-chloro-1-(1-methyl-1H-pyrazol-4-yl)pyridazin-4(1H)-one (Example #755 Step 3, 60 mg, 0.29 mmol) was added and the reaction mixture was heated to 50° C. for 4.5 hours. The reaction-mixture was then poured into water, extracted with EtOAc (3×), washed with brine, dried over MgSO₄, filtered and concentrated in vacuo. Purification of the residue by mass-triggered reverse-phase preparative HPLC gave 3-{[3-(5-ethoxypyrimidin-2-yl)phenyl]sulfanyl}-1-(1-methyl-1H-pyrazol-4-yl)pyridazin-4(1H)-one.

LRMS (ESI) calc'd for C20H19N6O2S [M+H]⁺: 407. Found: 407.

The alkyl halides and mesylate used in Scheme 6 for the synthesis of Examples #259, 269, 271-273, 290 and 345-348 and Scheme 25 for the synthesis of Examples #711-712 and 717-718 were prepared as follows:

### Intermediate #181

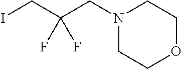

### 4-(2,2-Difluoro-3-iodopropyl)morpholine

### Step 1. 4-(2,2-Difluoro-3-iodopropyl)morpholine

Imidazole (359 mg, 5.27 mmol) was dissolved in DCM (8 mL) and iodine (1.34 g, 5.27 mmol) was added. The reaction mixture stirred at r.t. for 15 minutes and cooled to 0° C. Triphenylphosphine (1.30 g, 5.27 mmol) and a solution of 2,2-difluoro-3-(morpholin-4-yl)propan-1-ol (Intermediate #202, 382 mg, 2.10 mmol) in DCM (2 mL)/DMA (1.5 mL) were added and the reaction mixture stirred at r.t. for 48 hrs. The reaction mixture was diluted with EtOAc, washed with brine, and the aqueous phase was extracted with DCM. The organic layer was dried over Na₂SO₄, filtered and concentrated in vacuo while loading onto silica. Purification of the residue by flash chromatography (MPLC, 2-20% EtOAc-hexanes) gave 4-(2,2-difluoro-3-iodopropyl)morpholine as a colorless liquid.

LRMS (ESI) calc'd for C7H13F2INO [M+H]⁺: 292. Found: 292.

### Intermediate #182

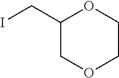

### rac-2-(Iodomethyl)-1,4-dioxane

### Step 1. rac-2-(Iodomethyl)-1,4-dioxane

rac-2-(Iodomethyl)-1,4-dioxane was prepared from rac-1,4-dioxan-2-ylmethanol (Intermediate #201) according to the procedure described for 4-(2,2-difluoro-3-iodopropyl)morpholine (Intermediate #181).

### Intermediate #183

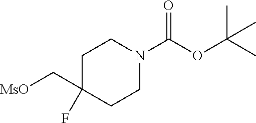

### tert-Butyl 4-fluoro-4-{[(methylsulfonyl)oxy]methyl}piperidine-1-carboxylate

### Step 1. tert-Butyl 4-fluoro-4-{[(methylsulfonyl)oxy]methyl}piperidine-1-carboxylate

tert-Butyl 4-fluoro-4-(hydroxymethyl)piperidine-1-carboxylate (Intermediate #197, 0.507 g, 2.173 mmol) and TEA (0.364 mL, 2.61 mmol) were taken up in DCM (5 mL) and cooled to 0° C. Methanesulfonyl chloride (0.186 mL, 2.39 mmol) was added and the resulting mixture stirred for 4 hours, during which time the temperature increased to ˜15° C. Saturated NH₄Cl was added and the products extracted into EtOAc (x2). The combined organic extracts were washed with brine, dried over Na₂SO₄ and concentrated in vacuo to give tert-butyl 4-fluoro-4-{[(methylsulfonyl)oxy]methyl}piperidine-1-carboxylate as a yellow solid.

LRMS (ESI) calc'd for C8H15FNO5S [M+H]⁺: 256. Found: 256 (carbamic acid).

### Intermediate #184

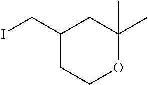

### rac-4-(Iodomethyl)-2,2-dimethyltetrahydro-2H-pyran

### Step 1. rac-4-(Iodomethyl)-2,2-dimethyltetrahydro-2H-pyran

rac-4-(Iodomethyl)-2,2-dimethyltetrahydro-2H-pyran was prepared from rac-(2,2-dimethyltetrahydro-2H-pyran-4-yl)methanol according to the procedure described for 4-(2,2-difluoro-3-iodopropyl)morpholine (Intermediate #181).

### Intermediate #185

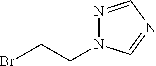

### 1-(2-Bromoethyl)-1H-1,2,4-triazole

### Step 1. 1-(2-Bromoethyl)-1H-1,2,4-triazole

2-(1H-1,2,4-Triazol-1-yl)ethanol (100 mg, 0.9 mmol) was dissolved in DCM (10 mL). CBr₄ (380 mg, 1.15 mmol) and PS-triphenylphosphine (1.2 g, 2.2 mmol) were added. The reaction mixture was heated to 40° C. and stirred for 1-8 hours. The reaction-mixture was filtered and the solvent was removed in vacuo to give 1-(2-bromoethyl)-1H-1,2,4-triazole as a crude oil.

### Intermediate #186

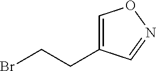

### 4-(2-Bromoethyl)isoxazole

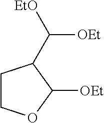

### Step 1. 3-(Diethoxymethyl)-2-ethoxytetrahydrofuran

To a stirred and cooled mixture of triethyl orthoformate (63 g, 0.425 mol) and boron diethyl trifluoroetherate (8.1 g, 0.06 mol) was added dihydrofuran (9 g, 0.129 mol). The reaction mixture was stirred at r.t. for 30 min. Diethanolamine (1 g, 0.01 mol) was added and the mixture was distilled to give 3-(diethoxymethyl)-2-ethoxytetrahydrofuran.

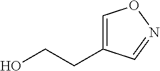

### Step 2. 2-(Isoxazol-4-yl)ethanol

3-(Diethoxymethyl)-2-ethoxytetrahydrofuran (32 g, 0.15 mol) was added at 50° C. to a solution of hydroxylamine hydrochloride (13.3 g, 0.19 mol) in water (50 mL). The mixture was stirred for 50 min and treated with sodium carbonate (30g). The mixture was extracted with diethyl ether (4×50 mL), the solvent was removed in vacuo and the residue distilled. The fraction with b.p. 120-121° C./4 mm Hg was collected to give 2-(isoxazol-4-yl)ethanol as an oil.

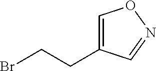

### Step 3. 4-(2-Bromoethyl)isoxazole

4-(2-Bromoethyl)isoxazole was prepared from 2-(isoxazol-4-yl)ethanol according to the procedure described for 1-(2-bromoethyl)-1H-1,2,4-triazole (Intermediate #185).

### Intermediate #187

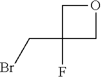

### 3-(Bromomethyl)-3-fluorooxetane

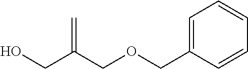

### Step 1. 2-[(Benzyloxy)methyl]prop-2-en-1-ol

2-Methylene-1,3-propanediol (2.0 mL, 25 mmol) and dibutyltin oxide (6.8 g, 27.5-mmol) were dissolved in CHCl₃ (90 mL)/MeOH (10 mL) and refluxed for 24 hrs. The reaction mixture was cooled to r.t. and the solvent was removed in vacuo to give a white solid which was dissolved in DMF (40 mL). Cesium fluoride (7.2 g; 47.4 mmol) and benzyl bromide (3.3 mL, 27.5 mmol) were added and the reaction mixture was stirred at r.t. for 24 hrs. The reaction was then heated to 50° C. for 1 hr then gradually cooled to r.t. The reaction mixture was diluted with EtOAc (100 mL) and water (10 mL) and was stirred for 30 minutes then filtered through Celite. The filtrate was washed with brine and the aqueous phase was extracted with EtOAc. The combined organic extracts were dried over Na₂SO₄, filtered and concentrated in vacuo while loading onto silica. Purification of the residue by flash chromatography (MPLC, 2-30% EtOAc-hexanes) to give 2-[(benzyloxy)methyl]prop-2-en-1-ol as a yellow oil.

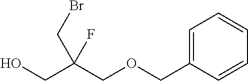

### Step 2. 3-(Benzyloxy)-2-(bromomethyl)-2-fluoropropan-1-ol

2-[(Benzyloxy)methyl]prop-2-en-1-ol was dissolved in DCM (12.5 mL) and triethylamine trihydrofluoride (0.69 mL, 4.2 mmol) was added followed by the portionwise addition of NBS (550 mg, 3.1 mmol) at −10° C. The resulting mixture was stirred for 4.5 hrs. The reaction mixture was then poured into ice-water (50 mL) and neutralized with ammonium hydroxide. The organic layer was separated and diluted with DCM then washed with 1 N HCl followed by saturated NaHCO₃ and the aqueous phase was extracted with DCM. The combined organic extracts were dried over Na₂SO₄, filtered and concentrated in vacuo while loading onto silica. Purification of the residue by flash chromatography (MPLC, 2-40% EtOAc-hexane) gave 3-(benzyloxy)-2-(bromomethyl)-2-fluoropropan-1-ol as a colorless oil.

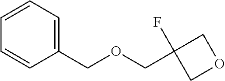

### Step 3. 3-[(Benzyloxy)methyl]-3-fluorooxetane

3-(Benzyloxy)-2-(bromomethyl)-2-fluoropropan-1-ol (1.1 g, 4.0 mmol) was dissolved in MeCN (20 mL) and K₂CO₃ (3.3 g, 24.0 mmol) was added. The reaction mixture was refluxed for 2 days. The reaction mixture was diluted with EtOAc, washed with brine and the aqueous phase was extracted with EtOAc. The combined organic extracts were dried over Na₂SO₄, filtered and concentrated in vacuo while loading onto silica. Purification of the residue by flash chromatography (MPLC, 2-20% EtOAc-hexanes) gave 3-[(benzyloxy)methyl]-3-fluorooxetane as a colorless oil.

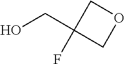

### Step 4. (3-Fluorooxetan-3-yl)methanol

To Pd/C (5%, 75 mg) under a nitrogen atmosphere was added a solution of 3-[(benzyloxy)methyl]-3-fluorooxetane (400 mg, 2.05 mmol) in EtOH (5 mL) and acetic acid (0.5 mL). The reaction mixture was stirred overnight under H₂ (40 psi). Upon completion, the reaction mixture was filtered through PL-HCO₃ cartridges (Stratospheres™, 0.9 mmol) and the solvent was removed in vacuo to give (3-fluorooxetan-3-yl)methanol.

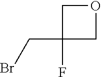

### Step 5. 3-(Bromomethyl)-3-fluorooxetane

3-(Bromomethyl)-3-fluorooxetane was prepared from (3-fluorooxetan-3-yl)methanol according to the procedure described for 1-(2-bromoethyl)-1H-1,2,4-triazole (Intermediate #185).

### Intermediate #188

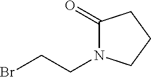

### 1-(2-Bromoethyl)pyrrolidin-2-one

### Step 1. 1-(2-Bromoethyl)pyrrolidin-2-one

1-(2-Hydroxyethyl)pyrrolidin-2-one (62 mg, 0.480 mmol), PS-triphenylphosphine (652 mg, 1.20 mmol), and CBr₄ (207 mg, 0.624 mmol) were combined in DCM (5 mL) and sealed in a pressure flask. The reaction mixture was allowed to stir at 45° C. for 18 hours. The mixture was filtered through Celite eluting with DCM (10 mL) and the solvent removed in vacuo to give crude 1-(2-bromoethyl)pyrrolidin-2-one that was used without further manipulation.

### Intermediate #189

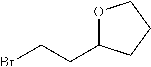

### rac-2-(2-Bromoethyl)tetrahydrofuran

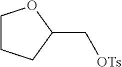

### Step 1. rac-Tetrahydrofuran-2-ylmethyl 4-methylbenzenesulfonate

To a solution of rac-tetrahydrofuran-2-ylmethanol (102g, 1 mol) and TsCl (190.6 g, 1 mol) in Et₂O (500 mL) was added KOH (84 g, 1.5 mol) in portions at 0° C. The resulting solution was stirred at ambient temperature for 2 hours. The solution was diluted with water (500 mL) and extracted with Et₂O (X2). The combined organic extracts were dried over Na₂SO₄, filtered and concentrated in vacuo to give rac-tetrahydrofuran-2-ylmethyl 4-methylbenzenesulfonate.

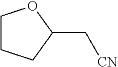

### Step 2. rac-Tetrahydrofuran-2-ylacetonitrile

A mixture of rac-tetrahydrofuran-2-ylmethyl 4-methylbenzenesulfonate (350 g, 0.819 mol) and KCN (93 g, 1.44 mol) in EtOH/H₂O (3:2, 500 mL) was heated to 90° C. overnight. The EtOH was removed in vacuo and the residue diluted with water and extracted with EtOAc (X2). The combined organic extracts were dried over Na₂SO₄, filtered and concentrated in vacuo. The residue was vacuum distilled to give rac-tetrahydrofuran-2-ylacetonitrile.

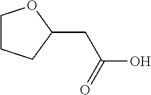

### Step 3. rac-Tetrahydrofuran-2-ylacetic acid

rac-Tetrahydrofuran-2-ylacetonitrile (50 g, 0.448 mol) in of 3 N NaOH:EtOH (2:1, 750 mL) was heated to 90° C. overnight. After cooling to r.t., the EtOH was removed in vacuo. The residue was extracted with EtOAc (X2). The aqueous phase was adjusted to pH 1 at 0° C. and extracted with EtOAc (X2). The combined organic extracts were dried over Na₂SO₄, filtered and concentrated in vacuo to give rac-tetrahydrofuran-2-ylacetic acid.

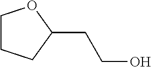

### Step 4. rac-2-(Tetrahydrofuran-2-yl)ethanol

To 300 mL Et₂O was added LAH (13.1 g, 0.346 mol) in portions at 0° C., followed by a solution of rac-tetrahydrofuran-2-ylacetic acid (30 g, 0.231 mol) in Et₂O (50 mL) dropwise at 0° C. The resulting solution was stirred at ambient temperature for 4 hours. After cooling to 0° C., the mixture was quenched with saturated Na₂SO₄ solution. The residue was dissolved in 4 N HCl (300 mL) and extracted with petroleum ether (X2). The aqueous phase was then extracted with CHCl₃. The combined CHCl₃ extracts were dried over Na₂SO₄, filtered, concentrated in vacuo and purified by distillation to afford rac-2-(tetrahydrofuran-2-yl)ethanol.

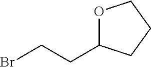

### Step 5. rac-2-(2-Bromoethyl)tetrahydrofuran

rac-2-(2-Bromoethyl)tetrahydrofuran was prepared from rac-2-(tetrahydrofuran-2-yl)ethanol according to the procedure described for 1-(2-bromoethyl)pyrrolidin-2-one (Intermediate #188).

### Intermediate #190

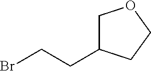

### rac-3-(2-Bromoethyl)tetrahydrofuran

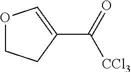

### Step 1. 2,2,2-Trichloro-1-(4,5-dihydrofuran-3-ylethanone

To a solution of 2,3-dihydrofuran (100 g, 1.43 mol) and pyridine (169 g, 2.14 mol) in CHCl₃ (600 mL) was added dropwise 2,2,2-trichloroacetyl chloride (389 g, 2.014 mol) at 0° C. After complete addition, the reaction mixture was stirred at r.t. for 18 hours. Saturated NaHCO₃ was added dropwise to quench the reaction at 0° C. The organic layer was separated, washed with water (X3), dried over Na₂SO₄, filtered and concentrated in vacuo to give 2,2,2-trichloro-1-(4,5-dihydrofuran-3-yl)ethanone.

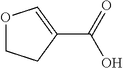

### Step 2. 4,5-Dihydrofuran-3-carboxylic acid

To a mixture of 2,2,2-trichloro-1-(4,5-dihydrofuran-3-yl)ethanone (280 g, 1.30 mol) and KOH (355 g, 5.20 mol) suspended in benzene (2.5 L) was added 20 drops of water. The reaction mixture was heated to 80° C. for 18 hours. The solid was filtered and dissolved in water and the products extracted into Et₂O (X2). The aqueous phase was then adjusted to pH 3 with 6 N HCl solution and extracted with DCM (X3). The combined DCM extracts were dried over Na₂SO₄, filtered and concentrated in vacuo to give 4,5-dihydrofuran-3-carboxylic acid as a yellow solid.

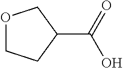

### Step 3. rac-Tetrahydrofuran-3-carboxylic acid

To a mixture of 4,5-dihydrofuran-3-carboxylic acid (50 g, 0.438 mol) in EtOAc (200 mL) was added 5g Pd/C. The reaction mixture was heated to 60° C. under H₂ (580 psi) for 18 hours. The mixture was filtered and the filtrate concentrated in vacuo to give rac-tetrahydrofuran-3-carboxylic acid.

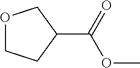

### Step 4. rac-Methyl tetrahydrofuran-3-carboxylate

To a mixture of rac-tetrahydrofuran-3-carboxylic acid (76 g, 0.654 mol) and K₂CO₃ (90.3 g, 0.654 mol) suspended in acetone (500 mL) was added dropwise dimethyl sulfate (82.5 g, 0.654 mol) at 0° C. After complete addition, the mixture was heated to 60° C. for 12 hours. The mixture was cooled to r.t. and the solvent removed in vacuo. The residue was dissolved in water (500 mL) and the products extracted into EtOAc (X3). The combined organic extracts were washed with water and brine, dried over Na₂SO₄, filtered and concentrated in vacuo to give rac-methyl tetrahydrofuran-3-carboxylate.

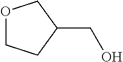

### Step 5. rac-Tetrahydrofuran-3-ylmethanol

To a mixture of LAH (19.8 g, 0.522 mol) in Et₂O (600 mL) was added dropwise a solution of rac-methyl tetrahydrofuran-3-carboxylate (68 g, 0.522 mol) in Et₂O (100 mL) at 0° C. under N₂ gas. After complete addition, the mixture was stirred at r.t. for 18 hours. The mixture was quenched slowly with water at 0° C. The resulting mixture was filtered and the filter cake was washed with EtOAc (300 mL×3). The organic phase was separated, dried over Na₂SO₄, filtered and concentrated in vacuo to give rac-tetrahydrofuran-3-ylmethanol.

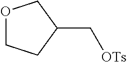

### Step 6. rac-Tetrahydrofuran-3-ylmethyl 4-methylbenzenesulfonate

To a solution of rac-tetrahydrofuran-3-ylmethanol (49 g, 0.480 mol) and TsCl (91.5 g, 0.480 mol) in Et₂O (500 mL) was added KOH (53.7 g, 0.960 mol) in portions at 0° C. The resulting solution was stirred at ambient temperature for 4 hours. The solution was diluted with water (500 mL). The organic layer was separated, washed with brine, dried over Na₂SO₄, filtered and concentrated in vacuo to give rac-tetrahydrofuran-3-ylmethyl 4-methylbenzenesulfonate.

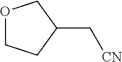

### Step 7. rac-Tetrahydrofuran-3-ylacetonitrile

A mixture of rac-tetrahydrofuran-3-ylmethyl 4-methylbenzenesulfonate (91 g, 0.355 mol) and KCN (34.6 g, 0.533 mol) in EtOH/H₂O (3:2, 500 mL) was heated to 90° C. overnight. The EtOH was removed in vacuo, the residue diluted with water and the products extracted into EtOAc (X3). The combined organic extracts were dried over Na₂SO₄, filtered and concentrated in vacuo. The residue was distilled to give rac-tetrahydrofuran-3-ylacetonitrile.

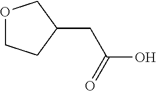

### Step 8. rac-Tetrahydrofuran-3-ylacetic acid

A mixture of rac-tetrahydrofuran-3-ylacetonitrile (65 g, 0.585 mol) and NaOH (65.6 g, 1.638 mol) in EtOH/H₂O (1:2, 885 mL) was heated to 90° C. overnight. The EtOH was removed in vacuo, the residue diluted with water and the products extracted into DCM (X3). The aqueous phase was acidified to pH 1-2 and extracted with EtOAc (X3). The combined EtOAc extracts were dried over Na₂SO₄, filtered and concentrated in vacuo to give rac-tetrahydrofuran-3-ylacetic acid.

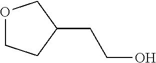

### Step 9. rac-2-(Tetrahydrofuran-3-yl)ethanol

To a mixture of LAH (30.7 g, 0.807 mol) in Et₂O (900 mL) was added dropwise a solution of rac-tetrahydrofuran-3-ylacetic acid (70 g, 0.538 mol) in Et₂O (100 mL) at 0° C. under N₂ gas. After complete addition, the mixture was stirred at r.t for 18 hours. The mixture was quenched slowly with water at 0° C. The resulting mixture was filtered and the filter cake was washed with EtOAc (300 mL×3). The organic phase was separated, dried over Na₂SO₄, filtered and concentrated in vacuo to give an oil. The residue was dissolved in water (400 mL) and extracted with petroleum ether (X3). The aqueous phase was concentrated in vacuo to afford rac-2-(tetrahydrofuran-3-yl)ethanol.

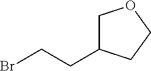

### Step 10. rac-3-(2-Bromoethyl)tetrahydrofuran

rac-3-(2-Bromoethyl)tetrahydrofuran was prepared from rac-2-(tetrahydrofuran-3-yl)ethanol according to the procedure described for 1-(2-bromoethyl)pyrrolidin-2-one (Intermediate #188).

The epoxides used in Scheme 6 for the synthesis of Examples #365, 367 and 373 were prepared as follows:

### Intermediate #191

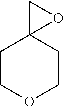

### 1,6-Dioxaspiro[2.5]octane

### Step 1. 1,6-Dioxaspiro[2.5]octane

1,6-Dioxaspiro[2.5]octane was prepared according to the procedure described in WO 2007/139569.

### Intermediate #192

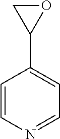

### 4-(Oxiran-2-yl)pyridine

### Step 1. 4-(Oxiran-2-yl)pyridine

Trimethylsulfoxonium iodide (1.73 g, 7.84 mmol) was dissolved in DMSO (13 mL) and cooled to 0° C. NaH (60 wt %, 341 mg, 7.84 mmol) was added and the reaction mixture was allowed to warm to r.t. before stirring for three hours. The reaction was once again cooled to 0° C. before pyridine-4-carbaldehyde (700 mg, 6.54 mmol) was added. The reaction mixture was then stirred overnight at r.t. Water was added and the products extracted into Et₂O. The combined organic extracts were washed with brine, dried over Na₂SO₄, filtered and concentrated in vacuo. Purification of the residue by flash chromatography (MPLC, 30-70% EtOAc-hexanes) gave 4-(oxiran-2-yl)pyridine.

LRMS (ESI) calc'd for C7H8NO [M+H]⁺: 122. Found: 122.

The alcohols used in Scheme 12 for the synthesis of Examples #513, 520, 524, 525, 568, 570, 601, 603, 609, 610 and 613 and Intermediates #162-165 were prepared as follows:

### Intermediate #193

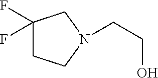

### 2-(3,3-Difluoropyrrolidin-1-yl)ethanol

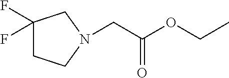

### Step 1. Ethyl (3,3-difluoropyrrolidin-1-yl)acetate

3,3-Difluoropyrrolidin hydrochloride (0.20 mL, 1.8 mmol), ethyl bromoacetate (523 mg, 3.64 mmol) and DIPEA (0.64 mL, 3.64 mmol) were dissolved in toluene (3 mL) and the reaction mixture was stirred at r.t. for 2 hrs. The reaction mixture was filtered, diluted with DCM and the solvent removed in vacuo while loading onto silica. Purification of the residue by flash chromatography (MPLC, 5-50% EtOAc-hexanes) gave ethyl (3,3-difluoropyrrolidin-1-yl)acetate as a yellow oil.

LRMS (ESI) calc'd for C8H14F2NO2 [M+H]⁺: 194. Found: 194.

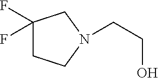

### Step 2. 2-(3,3-Difluoropyrrolidin-1-yl)ethanol

Ethyl (3,3-difluoropyrrolidin-1-yl)acetate (200 mg, 1.04 mmol) was dissolved in THF (4 mL) and cooled to 0° C., LiAlH₄ (1M in THF, 1.5 mL, 1.5 mmol) was added portionwise. The reaction mixture was stirred at 0° C. for one hour. The reaction was slowly quenched with Na₂SO₄.10 H₂O (1.6 g, 5.2 mmol). Additional THF (3 mL) was added and the reaction mixture was filtered through Celite. The filtrate was concentrated to a crude oil, diluted with DCM and the crude residue was purified by flash chromatography (MPLC, 1-10% MeOH-EtOAc) to give 2-(3,3-difluoropyrrolidin-1-yl)ethanol as a colorless oil.

### Intermediate #194

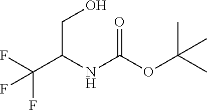

### rac-tert-Butyl (1,1,1-trifluoro-3-hydroxypropan-2-yl)carbamate

### Step 1. rac-tert-Butyl, (1,1,1-trifluoro-3-hydroxypropan-2-yl)carbamate

A vial was charged with rac-2-amino-3,3,3-trifluoropropan-1-ol (100 mg, 0.604 mmol), DCM (2 mL), TEA (0.168 mL, 1.21 mmol) and Boc₂O (0.168 mL, 0.725 mmol). The reaction mixture was stirred at r.t. for 12 hrs. DCM (5 mL) was added, followed by the addition of PL-PPZ MP-resin (336 mg, 1.21 mmol) and PL-HCO₃ MP-resin (613 mg, 1.21 mmol). The suspension was stirred at r.t. for 5 hrs. The reaction mixture was diluted with DCM and filtered. The solvent was removed in vacuo and the residue purified by flash chromatography (MPLC, 50% EtOAc-hexanes) to give rac-tert-butyl (1,1,1-trifluoro-3-hydroxypropan-2-yl)carbamate.

### Intermediate #195

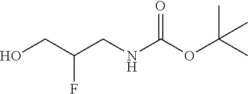

### rac-tert-Butyl (2-fluoro-3-hydroxypropyl)carbamate

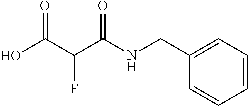

### Step 1. rac-3-(Benzylamino)-2-fluoro-3-oxopropanoic acid

To a stirred solution of diethyl fluoromalonate (600 g, 3.37 mol) in methanol (2.0 L) was added a solution of KOH (222 g, 3.37 mol) in methanol (1.5 L). The reaction mixture was stirred at room temperature for 2.5 hr. After this time period, benzylamine (1.1 L, 10.1 mol) was added and the resulting mixture stirred at 55° C. for 16 hr. The reaction mixture was concentrated in vacuo and diethyl ether (1.5 L) was added. The resulting slurry was filtered and rinsed with diethyl ether. The resulting solid was dissolved in 6N HCl (1.5 L) and extracted with EtOAc (2×1.0 L). The combined organic layers were washed with brine (1.0 L), dried over MgSO₄, filtered and concentrated in vacuo to afford rac-3-(benzylamino)-2-fluoro-3-oxopropanoic acid as a pale yellow solid.

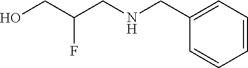

### Step 2. rac-3-(Benzylamino)-2-fluoropropan-1-ol

To a stirred solution of borane methylsulfide (1.1 L, 11.0 mol) in THF (3.8 L) was added a solution of rac-3-(benzylamino)-2-fluoro-3-oxopropanoic acid (580 g, 2.80 mol) in THF (2.0 L) dropwise under a nitrogen atmosphere. Upon complete addition, the reaction mixture was warmed to ambient temperature over 4 hr. The reaction mixture was then stirred overnight at 55° C. The reaction mixture was quenched by the sequential addition of water (380 mL) and 6N HCl (500 mL). The resulting solution was washed with EtOAc (2×1.0 L). The resulting aqueous layer was treated with 50% aqueous NaOH until the pH was basic. The aqueous layer was extracted with EtOAc. The organic layer was dried over MgSO₄, filtered and concentrated in vacuo to afford rac-3-(benzylamino)-2-fluoropropan-1-ol as a crude oil.

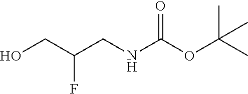

### Step 3. rac-tert-Butyl (2-fluoro-3-hydroxypropyl)carbamate

To a stirred solution of rac-3-(benzylamino)-2-fluoropropan-1-ol (440g 2.4 mol) in MeOH was added 10% Pd/C (52% water wet; 220g), followed by ammonium formate (760 g, 12 mol). The resultant suspension was stirred at 65° C. for 3 hr. The suspension was filtered through Celite and the filtrate was concentrated in vacuo. The crude residue was diluted with water (1.5 L) and the pH was adjusted to approximately 3-4 using concentrated HCl. The acidified solution was washed with EtOAc (2×1.0 L) and the pH of the aqueous phase was adjusted with 50% NaOH solution until basic. To this mixture was added a solution of Boc₂O (500 g, 2.3 mol) in THF (1.0 L) dropwise. Upon complete of addition, the reaction mixture was stirred overnight at r.t. The layers were separated and the aqueous layer was extracted with EtOAc (2×1.0 L). The combined organic layers were washed with brine (1.0 L), dried over MgSO₄, filtered, concentrated in vacuo and purified by flash chromatography (2:1 hexanes/EtOAc) to afford rac-tert-butyl (2-fluoro-3-hydroxypropyl)carbamate as a pale yellow oil.

The following intermediate was prepared from diethyl difluoromalonate following similar procedures described for Intermediate #195, which can be achieved by those of ordinary skill in the art of organic chemist.

| | | | Exact |
|---|---|---|---|
| | | | Mass |
| Intermediate | Structure | IUPAC Name | [M + H]⁺ |
| 196 | 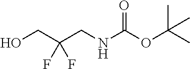 | tert-butyl (2,2- difluoro-3- hydroxypropyl) carbamate | nd. |

### Intermediate #197

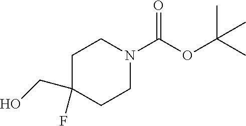

### tert-Butyl 4-fluoro-4-{[(methylsulfonyl)oxy]methyl}piperidine-1-carboxylate

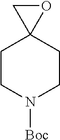

### Step 1. tert-butyl 1-oxa-6-azaspiro[2.5]octane-6-carboxylate

To a stirred and warm (−45° C.)-solution of tert-butyl 4-oxopiperidine-1-carboxylate (145.0 g, 0.73 moles) and trimethylsulfoxonium iodide (165.0 g, 0.75 moles) in anhydrous THF (1.1 L)/DMF (0.55 L) was added-solid potassium tert-butoxide (84.2 g, 0.75 moles) over several minutes. The resultant exotherm warmed the reaction mixture to −60° C., the mixture was then kept at −60° C. for an additional 30-40 minutes, then cooled to r.t. (overnight) and concentrated in vacuo. The resultant slurry was partitioned between EtOAc (0.9 L) and H₂O (0.9 L). The organic layer was separated, washed with H₂O (0.25 L, x2), dried over Na₂SO₄ and concentrated in vacuo. The resultant oil solidified upon standing in a refrigerator to give tert-butyl 1-oxa-6-azaspiro[2.5]octane-6-carboxylate as an off-white solid that was used in the next step directly.

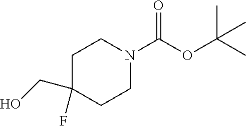

### Step 2. tert-Butyl 4-fluoro-4-(hydroxymethyl)piperidine-1-carboxylate

A cooled (−10° C.) solution of tert-butyl 1-oxa-6-azaspiro[2.5]octane-6-carboxylate (125 g, 0.59 mol) in anhydrous DCM (350 mL) was slowly treated with neat HF pyridine (˜80 mL, ˜2.5 mol) while maintaining the internal temperature below −5° C. The reaction mixture was stirred at −5° C. to −10° C. for 1.5 hours, warmed to ˜0° C. and slowly quenched with saturated Na₂CO₃ (400 mL). Additional solid Na₂CO₃ was added until CO evolution ceased and the mixture was passed through a coarse filter to remove the insoluble matter. The organic layer was separated, washed with H₂O (150 mL, ×2), 5% aqueous H₂SO₄ (150 mL) and saturated NaHCO₃ (150 mL), dried over Na₂SO₄ and concentrated in vacuo. The residual oil was purified by flash chromatography (2:1 hexanes/EtOAc with 2% Et₃N) to give tert-butyl 4-fluoro-4-(hydroxymethyl)piperidine-1-carboxylate as a viscous pale yellow oil.

### Intermediate #198

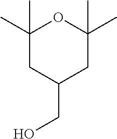

### (2,2,6,6-Tetramethyltetrahydro-2H-pyran-4-yl)methanol

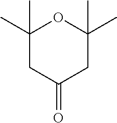

### Step 1,2,2,6,6-Tetramethyltetrahydro-4H-pyran-4-one

1 N HCl (307 mL, 0.3 mol) was added to a solution of 2,6-dimethylhepta-2,5-dien-4-one (30 g, 0.2 mol) in THF (200 mL). The reaction mixture was heated to 40° C. and stirred for 3 days. The mixture was extracted with ether. The organic layer was dried over Na₂SO₄, filtered and concentrated in vacuo. The crude residue was purified by flash chromatography to give 2,2,6,6-tetramethyl-2H-3,5,6-trihydropyran-4-one.

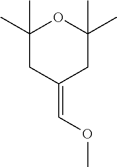

### Step 2. 4-(Methoxymethylene)-2,2,6,6-tetramethyltetrahydro-2H-pyran

(Methoxymethyl)triphenylphosphonium chloride (46.5 g, 0.14 mol) was dissolved in THF (180 mL) and stirred under N₂ at 0° C. t-BuOK (11.4 g, 0.1 mol) was dissolved in THF (130 mL) and added via syringe to the reaction mixture. The mixture was stirred at 0° C. for 0.5 h. The mixture was cooled to −30° C. and a solution of 2,2,6,6-tetramethyl-2H-3,5,6-trihydropyran-4-one (10.6 g, 0.07 mol) in THF (50 mL) was added. The reaction mixture was stirred at −30° C. for 1 h. The mixture was warmed to r.t., water was added and the mixture was stirred for 0.5 h. The solvent was removed in vacuo and the precipitate was cooled by ice-water. The solid was filtered off and the filtrate was extracted with Et₂O. The organic layer was concentrated to 30 mL and cooled with ice. A white precipitate formed. The mixture was filtered and the filtrate was concentrated to afford 4-(methoxymethylene)-2,2,6,6-tetramethyltetrahydro-2H-pyran.

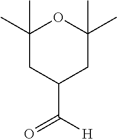

### Step 3. 2,2,6,6-Tetramethyl-2H-3,4,5,6-tetrahydropyran-4-carbaldehyde

4-(Methoxymethylene)-2,2,6,6-tetramethyltetrahydro-2H-pyran (10.5 g, 0.06 mol) was dissolved in formic acid (60 mL) and the reaction mixture was stirred at r.t-overnight. The mixture was diluted with water and neutralized with aqueous Na₂CO₃. The aqueous layer was extracted with ether, the solvent removed in vacuo, and the crude residue was purified by flash chromatography to afford 2,2,6,6-tetramethyl-2H-3,4,5,6-tetrahydropyran-4-carbaldehyde.

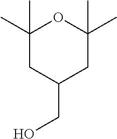

### Step 4. (2,2,6,6-Tetramethyltetrahydro-2H-pyran-4-yl)methanol

2,2,6,6-Tetramethyl-2H-3,4,5,6-tetrahydropyran-4-carbaldehyde (5 g, 0.03 mol) was dissolved in EtOH (50 mL) and NaBH₄ (1.7 g, 0.046 mol) was added. The reaction mixture was stirred at r.t. for 4 h. The solvent was removed in vacuo and the residue was purified by flash chromatography (petroleum ether: EtOAc=10:1) to give (2,2,6,6-tetramethyltetrahydro-2H-pyran-4-yl)methanol.

### Intermediate #199

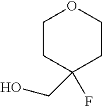

### (4-Fluorotetrahydro-2H-pyran-4-yl)methanol

(4-Fluorotetrahydro-2H-pyran-4-yl)methanol was prepared according to the procedure described in WO 2006034093.

### Intermediate #200

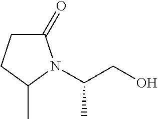

### (5R or S)-1-[(2S)-1-Hydroxypropan-2-yl]-5-methylpyrrolidin-2-one (Enantiomer B)

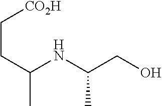

### Step 1. 4-{[(2S)-1-Hydroxypropan-2-yl]amino}pentanoic acid

(S)-(+)-2-Amino-1-propanol (12.7 g, 0.17 mol) was dissolved in EtOH (125 mL) and 4-oxopentanoic acid (17.2 g, 0.15 mol) was slowly added. PtO₂ (0.53 g, 0.002 mol) was then added and the reaction-mixture was stirred under H₂ (40 psi) overnight. Upon completion, the reaction mixture was diluted with MeOH and filtered through Celite. The filtrate was concentrated in vacuo to give 4-{[(2S)-1-hydroxypropan-2-yl]amino}pentanoic acid a white solid.

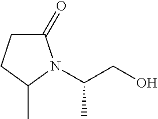

### Step 2. 1-[(2S)-1-Hydroxypropan-2-yl]-5-methylpyrrolidin-2-one

4-{[(2S)-1-Hydroxypropan-2-yl]amino}pentanoic acid (41 g, 0.23 mol) was dissolved in toluene (600 mL) and refluxed under Dean-Stark conditions overnight. The reaction was cooled to r.t. and concentrated in vacuo and the crude residue was purified by flash chromatography (1:1 hexanes-acetone) to give 1-[(2S)-1-hydroxypropan-2-yl]-5-methylpyrrolidin-2-one as a yellow oil.

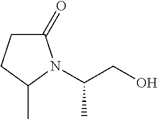

### Step 3. (5R or S)-1-[(2S)-1-Hydroxypropan-2-yl]-5-methylpyrrolidin-2-one (Stereoisomer B)

1-[(2S)-1-Hydroxypropan-2-yl]-5-methylpyrrolidin-2-one (28 g, 0.18 mol) was subjected to chiral preparative SFC (Berger Multigram II SFC, column: ChiralPak AD 2.5×25 cm, 5 μM, mobile phase: 20% EtOH/80% heptane, flow rate: 500 mL/min). The fractions were collected and the solvent evaporated in vacuo to give (5R or S)-1-[(2S)-1-hydroxypropan-2-yl]-5-methylpyrrolidin-2-one (Stereoisomer B).

### Intermediate #201

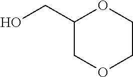

### rac-1,4-Dioxan-2-ylmethanol

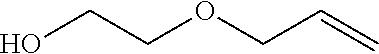

### Step 1. 2-(Allyloxy)ethanol

In a 2 L 3-neck flask equipped with stirrer, thermometer and dropping funnel was added glycol (1335 g, 21.5 mol). To the glycol was added KOH (144.7 g, 2.58 mol), keeping the temperature at 0-5° C. Then the mixture was stirred until the KOH was all dissolved in the glycol. To the solution was added drop-wise allyl bromide (260.2 g, 2.15 mol), keeping the temperature at 0-5° C. over a period of 3 hours. Then the mixture was allowed to stir for two days at ambient temperature. The reaction mixture was diluted with water (2 L) and extracted with EtOAc (X3). The combined organic extracts were dried over Na₂SO₄, filtered and concentrated in vacuo. The residue was distilled to afford 2-(allyloxy)ethanol as a colorless liquid (90° C./30 mmHg), which was used in the next step without further purification.

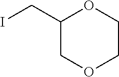

### Step 2. rac-2-(Iodomethyl)-1,4-dioxane

A mixture of Hg(OAc)₂ (638 g, 2 mol) and 2-(allyloxy)ethanol (204 g, 2 mol) dissolved in water (1.5 L) was heated to 100 C and was stirred for 30 minutes. The reaction mixture was then cooled to room temperature. To the solution was added 2 N KOH (1 L, 2 mol) followed by aqueous KI (1.5 L, 2 mol) with stirring. A white precipitate formed and was filtered and washed with water. The wet solid was collected and dissolved in chloroform (4 L) and to the solution was added iodine (508 g, 2 mol). The mixture was stirred overnight at room temperature. After removal of the red solid, the filtrate was washed with aqueous Na₂S₂O₃ until it became colorless. This was dried over Na₂SO₄, filtered and concentrated in vacuo. The residue was purified by flash chromatography (EtOAc/petroleum ether=1:10) to afford rac-2-(iodomethyl)-1,4-dioxane as a colorless liquid.

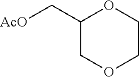

### Step 3. rac-2-Acetoxymethyl-1,4-dioxane

A mixture of rac-2-iodomethyl-1,4-dioxane (230g, 1 mol), AgOAc (250 g, 1.5 mol) and HOAc (1.2 L) was heated to 120° C. overnight. After cooling to room temperature, a precipitate formed that was filtered and the filtrate was diluted with Et₂O (3 L). The solution was then neutralized with aqueous K₂CO₃. The aqueous phase was separated and extracted with Et₂O (X3). The combined organic extracts were dried over Na₂SO₄, filtered and concentrated in vacuo. The residue was purified by flash chromatography (EtOAc/petroleum ether 1:10) to afford rac-2-acetoxymethyl-1,4-dioxane as a colorless liquid.

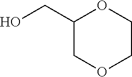

### Step 4. rac-1,4-Dioxan-2-ylmethanol

A mixture of rac-2-acetoxymethyl-1,4-dioxane (34.2 g, 0.21 mol), LiOH (18.0 g, 0.43 mol) and methanol (150 mL) was stirred at room temperature. The reaction was monitored by TLC until the starting material was all consumed. Then the mixture was evaporated to remove the methanol and the residue was diluted with Et₂O. The salt was filtered and the filtrate was evaporated. The residue was distilled to afford rac-1,4-dioxan-2-ylmethanol as a colorless liquid (100° C./11 mmHg).

LRMS (ESI) calc'd for C5H11O3 [M+H]⁺: 119. Found: 119.

### Intermediate #202

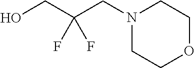

### 2,2-Difluoro-3-(morpholin-4-yl)propan-1-ol

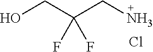

### Step 1. 3-Amino-2,2-difluoropropan-1-ol hydrochloride

tert-Butyl (2,2-difluoro-3-hydroxypropyl)carbamate (Intermediate #196, 100 mg, 0.473 mmol) was dissolved in 1,4-dioxane (1 mL) and 4 M HCl in 1,4-dioxane (2 mL) and the mixture stirred at r.t. for 2 hours. The solvent was removed in vacuo to afford 3-amino-2,2-difluoropropan-1-ol hydrochloride.

LRMS (ESI) calc'd for C3H8F2NO [M+H]⁺: 112. Found: 112.

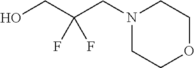

### Step 2. 2,2-Difluoro-3-(morpholin-4-yl)propan-1-ol

3-Amino-2,2-difluoropropan-1-ol hydrochloride (608 mg, 4.12 mmol), bis(2-bromoethyl)ether (1.05 g, 4.53 mmol) and DIPEA (2.88 mL, 16.5 mmol) were dissolved in DMF (10 mL) and heated to 65° C. for 12 hrs. The reaction mixture was diluted with EtOAc and washed with brine. The organic layer was dried over Na₂SO₄, filtered and concentrated in vacuo while loading onto silica. Purification of the residue by flash chromatography (MPLC, 12-100% EtOAc-hexanes) gave 2,2-difluoro-3-(morpholin-4-yl)propan-1-ol as a yellow oil.

### Intermediate #203

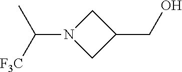

### rac-[1-(2,2,2-Trifluoro-1-methylethyl)azetidin-3-yl]methanol

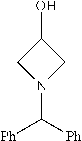

### Step 1. 1-(Diphenylmethyl)azetidin-3-ol

A solution of 2-(chloromethyl)oxirane (250 g, 2.346 mol) and 1,1-diphenylmethanamine (430 g, 2.346 mol) in MeOH (1000 mL) was stirred at ambient temperature for 3 days and then heated to reflux for a further 3 days. After concentration of the reaction mixture under reduced pressure, the residue was washed with acetone and filtered to give the HCl salt of 1-(diphenylmethyl)azetidin-3-ol.

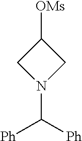

### Step 2. 1-(Diphenylmethyl)azetidin-3-yl methanesulfonate

To a solution of 1-(diphenylmethyl)azetidin-3-ol hydrochloride (276 g, 1 mol) in dry DCM (2000 mL) was added TEA (303 g, 3 mol) at 000° C. under nitrogen. After stirring for five minutes, MsCl (137.5 g, 1.2 mol) was added and the mixture was stirred for one hour. Water (1000 mL) was then added and the mixture extracted with DCM (1000 mL×3). The combined organic extracts were dried over MgSO₄, filtered and concentrated in vacuo. Purification of the residue by flash column (2% MeOH-DCM) gave 1-(diphenylmethyl)azetidin-3-yl methanesulfonate as a white solid.

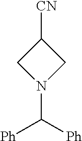

### Step 3. 1-(Diphenylmethyl)azetidine-3-carbonitrile

To a solution of 1-(diphenylmethyl)azetidin-3-yl methanesulfonate (95.1 g, 0.3 mol) in DMF (600 mL) was added a solution of KCN (22.1 g, 0.45 mol) in water (73 mL). The mixture was heated at 70° C. with stirring for 24 hrs, cooled and poured into ice-water. The precipitate was collected and dissolved in DCM (400 mL). Filtration of the dried organic solution through silica-gel and concentration in vacuo gave 1-(diphenylmethyl)azetidine-3-carbonitrile.

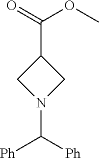

### Step 4. Methyl 1-(diphenylmethyl)azetidine-3-carboxylate

To a solution of 1-(diphenylmethyl)azetidine-3-carbonitrile (70 g, 0.28 mol) in MeOH (300 mL) was added dropwise conc. H₂SO₄ (180 mL), then the mixture was stirred for two hours at 90° C. Room temperature was attained and the mixture was poured into water (1000 mL). The aqueous mixture was basified with 6 N NaOH solution and the products were extracted into DCM (3×500 mL). The combined organic extracts were concentrated in vacuo and the residue purified by flash chromatography to give methyl 1-(diphenylmethyl)azetidine-3-carboxylate as a white solid.

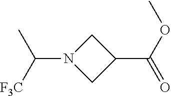

### Step 5. rac-Methyl 1-(2,2,2-trifluoro-1-methylethyl)azetidine-3-carboxylate

A mixture of methyl 1-(diphenylmethyl)azetidine-3-carboxylate (281 g, 1.0 mol), 10% Pd/C (50g) and 1,1,1-trifluoropropan-2-one (200 g, 1.79 mol) in MeOH (1500 mL) was agitated at 55° C. under H₂ (50-60 psi) overnight. The catalyst was removed by filtration and the solvent was removed in vacuo. The residue was distilled to obtain methyl T-(2,2,2-trifluoro-1-methylethyl)azetidine-3-carboxylate as an oil.

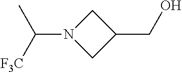

### Step 6. rac-[1-(2,2,2-Trifluoro-1-methylethyl)azetidin-3-yl]methanol

To a solution of rac-methyl 1-(2,2,2-trifluoro-1-methylethyl)azetidine-3-carboxylate (40g, 0.19 mol) in THF (400 mL) at 0° C. was added LiAlH₄ (7.2 g, 0.19 mol) portionwise over one hour. Water was then carefully added to the mixture at 0° C., then filtered. The solvent was dried over MgSO₄, filtered and concentrated in vacuo. The residue was distilled to obtain [1-(2,2,2-trifluoro-1-methylethyl)azetidin-3-yl]methanol.

The aryl halides used in Scheme 23 for the synthesis of Examples #679-681, 684-690, 692 and 694 and Intermediate #180 were prepared as follows:

### Intermediate #204

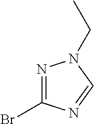

### 3-Bromo-1-ethyl-1H-1,2,4-triazole

### Step 1. 3-Bromo-1H-ethyl-1H-1,2,4-triazole

3-Bromo-1H-1,2,4-triazole (1 g, 6.76 mmol) was dissolved in DMF (10 mL) and THF (10 mL). While stirring at ambient temperature, NaH (60 wt %, 0.297 g, 7.43 mmol) was added portion-wise into the flask. After stirring for 15 minutes, iodoethane (0.601 mL, 7.43 mmol) was added and stirring at room temperature continued for an additional 2 hours. Saturated NH₄Cl (50 mL) was added and the products were extracted into EtOAc (X3). The combined organic extracts were washed with brine, dried over Na₂SO₄, filtered and concentrated in vacuo. Purification of the residue by flash chromatography (MPLC, 0-25%, EtOAc-hexanes) and collection of the first eluting regioisomer (major product) gave 3-bromo-1-propyl-1H-1,2,4-triazole.

LRMS (ES) calc'd for C4H7BrN3 [M+H]⁺: 176. found 176.

The following intermediate was prepared following similar procedures described for Intermediate #204, which can be achieved by those of ordinary skill in the art of organic chemistry.

| | | | Exact |
|---|---|---|---|
| | | | Mass |
| Intermediate | Structure | IUPAC Name | [M + H]⁺ |
| 205 | 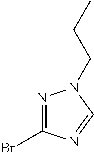 | 3-bromo-1-propyl- 1H-1,2,4-triazole | Calc'd 190, found 190 |

Intermediate #206

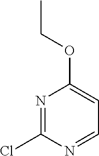

### Step 1. 2-Chloro-4-ethoxypyrimidine

To a solution of 2,4-dichloropyrimidine (300 mg. 2.014 mmol) in DMF (6.8 mL) were added EtOH (0.11 mL, 2.416 mmol) and Cs₂CO₃ (4.1 g, 3.42 mmol). The reaction mixture was stirred for 18 hours at 80° C. Room temperature was attained, saturated NaHCO₃ was added and the products extracted into EtOAc. The organic extract was dried, filtered and concentrated in vacuo. Purification of the residue by flash chromatography (MPLC, 0-30 EtOAc-hexanes) gave 2-chloro-4-ethoxypyrimidine.

LRMS (ESI) calc'd for C6H8ClN2O [M+H]⁺: 159. Found: 159.

The following intermediate was prepared following similar procedures described for Intermediate #206, which can be achieved by those of ordinary skill in the art of organic chemistry.

| | | | Exact |
|---|---|---|---|
| | | | Mass |
| Intermediate | Structure | IUPAC Name | [M + H]⁺ |
| 207 | 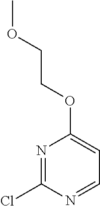 | 2-chloro-4-(2- methoxyethoxy) pyrimidine | Calc'd 189, found 189 |

### Intermediate #208

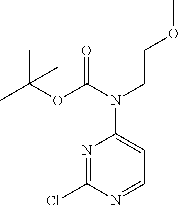

### tert-Butyl (2-chloropyrimidin-4-yl)(2-methoxyethyl)carbamate

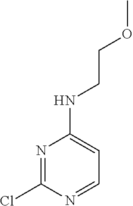

### Step 1. 2-Chloro-N-(2-methoxyethyl)pyrimidin-4-amine

### 2-Chloro-N-(2-methoxyethyl)pyrimidin-4-amine was prepared from 2,4-dichloropyrimidine and 2-methoxyethanamine according to the procedure described for 2-chloro-4-ethoxypyrimidine (Intermediate #206)

LRMS (ESI) calc'd for C7H11ClN3O [M+H]⁺: 188. Found: 188.

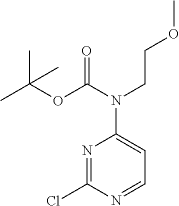

### Step 2. tert-Butyl (2-chloropyrimidin-4-yl)(2-methoxyethyl)carbamate

To a solution of 2-chloro-N-(2-methoxyethyl)pyrimidin-4-amine (170 mg, 0.906 mmol) and DMAP (111 mg, 0.906 mmol) in DCM (4.5 mL) were added TEA (0.126 mL, 0.906 mmol) and Boc₂O (237 mg, 1.087 mmol). The reaction mixture was stirred at r.t. for 1 h. The solvent was removed in vacuo and the residue purified by flash chromatography (MPLC, 50% EtOAc-hexanes) to afford tert-butyl (2-chloropyrimidin-4-yl)(2-methoxyethyl)carbamate as a white solid.

LRMS (ESI) calc'd for C12H19ClN3O₃[M+H]⁺: 288. Found: 288.

### Intermediate #209

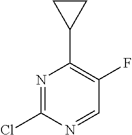

### 2-Chloro-4-cyclopropyl-5-fluoropyrimidine

### Step 1. 2-Chloro-4-cyclopropyl-5-fluoropyrimidine

5-Fluoro-2,4-dichloropyrimidine (5g, 29.9 mmol), cyclopropyl boronic acid (2.57 g, 29.9 mmol), K₃PO₄ (15.89 g, 74.9 mmol) and PdCl₂(dppf).DCM adduct (1.22 g, 1.50 mmol) were added to a dry flask. The flask was degassed with argon and then THF (150 mL) was added. The reaction mixture was degassed with argon for five minutes, and then heated to reflux for 12 hours. The reaction mixture was cooled to r.t., diluted with EtOAc (1000 mL), washed with brine, dried over MgSO₄, filtered and concentrated in vacuo. The residue was purified by flash chromatography (EtOAc-hexanes gradient) to afford 2-chloro-4-cyclopropyl-5-fluoropyrimidine.

LRMS (ESI) calc'd for C7H7ClFN2 [M+H]⁺: 173. Found: 173.

### Intermediate #210

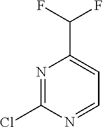

### 2-Chloro-4-(difluoromethyl)pyrimidine

### Step 1. 2-Chloro-4-(difluoromethyl)pyrimidine

Into a 500-mL 3-necked round-bottom flask, purged and maintained with an inert atmosphere of nitrogen, was placed a solution of 2-chloropyrimidine-4-carbaldehyde (15.0 g, 104 mmol) in DCM (200 mL). This was followed by the addition of bis[(2-methoxyethyl)amino]sulfur trifluoride (46.0 g, 208 mmol) dropwise with stirring at 0° C. over 30 min. The resulting solution was stirred for 2 h at 0° C., and then quenched by the addition of 50 mL of water. The resulting solution was extracted with DCM (3×100 mL). The combined organic extracts were dried over Na₂SO₄, filtered and concentrated in vacuo. Purification of the residue by flash chromatography (2:1 DCM-pentane) gave 2-chloro-4-(difluoromethyl)pyrimidine as a yellow oil.

LRMS (ESI) calc'd for C5H4ClF2N2 [M+H]⁺: 165. Found: 165.

### Intermediate #211

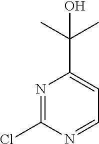

### 2-(2-Chloropyrimidin-4-yl)propan-2-ol

### Step 1. 2-(2-Chloropyrimidin-4-yl)propan-2-ol

To a solution of THF (0.25 mL) and toluene (1 mL) at −20° C. under a nitrogen atmosphere was added methyl magnesium chloride (3.0 M in THF, 1 mL, 2.90 mmol) followed by t-BuOH (0.050 mL in 0.750 mL THF, 0.579 mmol) and left to stir for 30 min at 0° C. The solution was cooled back down to −20° C. and methyl 2-chloropyrimidine-4-carboxylate in THF (1 mL) was added. The solution was warmed to room temperature and stirred for an additional 30 min. The solution was diluted with EtOAc, washed with brine, dried over MgSO₄, filtered and concentrated in vacuo to afford 2-(2-chloropyrimidin-4-yl)propan-2-ol.

LRMS (ESI) calc'd for C7H10ClN2O [M+H]⁺: 173. Found: 173.

### Pharmaceutical Composition

As a specific embodiment of this invention, 100 mg of 3-[3-(5-ethoxypyrimidin-2-yl)benzyl]-1-(1-methyl-1H-pyrazol-4-yl)pyridazin-4(1H)-one is formulated with sufficient finely divided lactose to provide a total-amount of 580 to 590 mg to fill a size 0, hard-gelatin capsule.

### Assays

The compounds of the instant invention described in Examples 1-756 were tested in the in vitro kinase assay I described below and were found to have MET inhibitory activity (IC₅₀<3000 nM, data shown below). Other assays are known in the literature and could be readily performed by those skilled in the art (see, for example, U.S. Patent Application Publications US 2005/0075340 A1, Apr. 7, 2005, pages 18-19; and PCT Publication WO 2005/028475, Mar. 31, 2005, pages 236-248). Representative cell-based assays II-IV can be used to further evaluate the biochemical and functional inhibitory potencies of test compounds and are provided for reference only.

### I. In Vitro Kinase Assay

The kinase activities of c-Met are measured using a modified version of the homogeneous-time-resolved tyrosine kinase assay described by Park et al. (1999, Anal. Biochem. 269:94-104).

The procedure for determining the potency of a compound to inhibit c-Met kinase comprises the following steps:
1. Prepare 3-fold serial diluted compound solutions in 100% dimethyl sulfoxide (DMSO) at 20× of the desired final concentrations in a 384-well plate.
2. Prepare a master reaction mix containing 6.67 mM MgCl₂, 133.3 mM NaCl, 66.7 mM Tris-HCl (pH 7.4), 0.13 mg/ml BSA, 2.67 mM dithiothreitol, 0.27 nM recombinant c-Met and 666.7 nM biotinylated synthetic peptide substrate (biotin-ahx-EQEDEPEGDYFEWLE-CONH₂) (SEQ.ID.NO.:1).
3. In a black assay plate, add 1.5 μl of compound solution (or DMSO) and 22.5 μl of master reaction mix per well. Initiate the kinase reaction by adding 6 μl of 0.25 mM MgATP per well. Allow the reactions to proceed for 60 min at room temperature. The final conditions for the reaction are 0.2 nM c-Met, 0.5 μM substrate, 50 μM MgATP, 5 mM MgCl₂, 100 mM NaCl, 2 mM DTT, 0.1 mg/ml BSA, 50 mM Tris (pH 7.4) and 5% DMSO.
4. Stop the kinase reaction with 30 μl of Stop/Detection buffer containing 10 mM EDTA, 25 mM HEPES, 0.1% TRITON X-100, 0.750 nM Eu-chelate labeled anti-phosphotyrosine antibody PY20 (cat. #AD0067, PerkinElmer) and 50 μg/ml Streptavidin-allophycocyanin conjugate (cat. #PJ25S, Prozyme).
5. Read HTRF signals on a Victor reader (PerkinElmer) in HTRF mode after 60-min incubation at room temperature in the dark.
6. IC₅₀ is determined by fitting the observed relationship between compound concentration and HTRF signal with a 4-parameter logistic equation.
   II. GTL-16 pY1349 Cell-Based Assay

The ability of compounds to inhibit the phosphorylation of Met Y1349 in GTL-16 cells (Ponzetto et al. Oncogene 1991; 6:553-559.) was measured using a 384-well AlphaScreen (Perkin Elmer) assay. GTL-16 cells were grown in RPMI Medium 1640 (no phenol red Invitrogen Cat #11835) with 10% FBS, 1% sodium pyruvate and 1% HEPES (pH 7.5). On day one, GTL-16 cells were seeded at a density of 10,000 cells/well in 20 ul of RPMI growth medium on Perkin Elmer CulturePlates. Plates were incubated at 37° C., 5% CO₂ overnight. The next day, 20 nL of serially diluted compounds were added to the cell plate via acoustic dispensing. Final compound concentrations of the 9-point 1:3 serial dilutions ranged from 10 μM to 1.5 nM. Cells were incubated in the presence of compound for 60 min at 37° C., 5% CO₂. After incubation, 20 uL of culture media were removed and 10 uL/well lysis buffer (30 mM Tris-HCL (pH 7.5), 5 mM EDTA, 50 mM NaCl, 30 mM NaPPi, 50 mM NaE, 0.5% (vol/vol) IGEPAL CA-630, 1% (vol/vol) Triton X-100, 10% glycerol, Roche Mini-Complete™ (without EDTA) protease inhibitor cocktail, 0.5 mM Na₃VO₄, 0.1 mg mL⁻¹ potassium bisperoxo (1,10-phenanthroline) oxovanadate (bpV-phen), 1% (vol/vol) phenylmethylsulfonyl fluoride (PMSF) and 0.5 mg mL⁻¹ Microcystin-LR) containing 1 ug/mL biotinylated anti-HGFR (R&D System, Cat #BAF358) was added to each well. Next, 10 uL of 5 ug/mL anti-phospho-Met Tyr1349 (Cell Signaling Technology, Cat#3121) in PBS plus 0.1% BSA was added to each well. Plates were then incubated at room temperature with shaking for 2 h. After incubation, 10 uL/well anti-IgG (Protein A) acceptor and streptavidin donor AlphaScreen bead mixture (50 ug/mL acceptor, 120 ug/mL donor; PerkinElmer, Cat#: 6760617R) in PBS with 0.1% BSA was added and the plates were incubated in the dark for 2 h. The AlphaScreen signal was read on an Envision (Perkin Elmer). After background correction, and normalization to untreated controls, the percent inhibition of Y1349 phosphorylation at each compound concentration was calculated. The plot of percent inhibition vs. the log of compound concentration was fit with a 4-parameter dose response equation to calculate IC₅₀ values.

### III. GTL-16 and HCT116 Proliferation Assay

The ability of compounds to inhibit the growth of GTL-16 cells with constitutively active amplified cMet (Ponzetto et al. Oncogene 1991; 6:553-559.) was assessed using an assay which measures cellular ATP levels as a proxy for viable cell mass. The assay makes use of a bioluminescent method from Lonza (Cat #LT07-321). In the presence of ATP, luciferase converts luciferin to oxyluciferin and light. The amount of light produced (emission at 565 nM) is measured and correlates with a relative amount of proliferation. A negative control cell line; HCT116 (ATCC #CCL-247), the growth of which is not dependent on met activity, was grown in 90% DMEM, 10% FBS, 10 mM HEPES pH 7.5. Two days prior to compound treatment, a 80-90% confluent flask of GTL-16 cells was split 1:4 in Complete Media and incubated in 5% CO₂ at 37° C. overnight. One day prior to compound treatment, GTL-16 cells at 1000 cells/well and HCT116 at 1000 cells/well were seeded in 20 uL complete medium in 384 well Perkin Elmer CulturePlates. Cells were incubated in the cell plates at 37° C., 5% CO₂ overnight. The next day, 100 mL of serially diluted compounds to the cell plate via acoustic dispensing. Cells were then incubated in the presence of compound for 72 hr at 37° C., 5% CO₂. At the end of the incubation, cells were lysed and ATP content was measured following the manufacturer's instructions. Assay plates were read in a luminometer after 2 min (1 sec exposure per well). The highest final compound concentration in the assay plates was 50 μM for test compounds, which were serially diluted 1:3 to give a final concentration series of 50000, 16667, 5556, 1852, 617, 206, 69, 23 and 7.6 nM. Final DMSO concentration was 0.5% in each well. The percentage inhibition of cell viability was calculated relative to untreated controls, plotted as a function of the log of compound concentration and analyzed using a four parameter logistical fit to calculate IC₅₀ values.

### IV. HPAF Scatter Assay

The ability of compounds to inhibit the HGF-dependent scattering phenotype of HPAF-II cells was measured using a modified version of the assay described by Chan et al. 2008 (Chan et al. J. Biomolec. Screening 2008; 13:847-854). Briefly, HPAF-II cells (ATCC #CRL1997) were plated in 50 uL DMEM (#11995)+10%-FBS+P/S at a density of 3,000 cells/well in Costar black clear bottom 384-well plates [Product no. 3712] and incubated at 37° C. overnight. The next day, 100 mL of serially diluted compound in DMSO was added to each well in the cell plate to give a nine-point 1:3 dilution series with final concentrations ranging from 20 μM to 3 nM. Cells were preincubated with compound at 37° C. for one hour. To stimulate the scattering phenotype, 10 uL of 24 ng/mL HGF (R&D Systems 294-HGN) was next added to each well, giving a final concentration of 4 ng/mL HGF. Cells were incubated with both compound and HGF at 37° C. for an additional 22 hrs. Control HGF-stimulated wells without compound treatment and control wells without HGF or compound were included on each plate. Next, to visualize the cells, each plate was washed in PBS IX, fixed in ice cold methanol for 3 min at RT, washed in PBS 3×, stained with Hoechst (1:2500) in PBS/0.1% Triton for 15 min in the dark, and finally washed in PBS 4× before imaging on an INCell Analyzer 1000 (GE Healthcare). Individual cell by cell SOI internuclear distance information was exported and then processed using a Pipeline Pilot (Accelrys) protocol to calculate the percentage of scattered cells. The percent inhibition of the scattering phenotype was calculated relative to cells without compound treatment, plotted against the log of compound concentration and then fit to a four parameter logistic fit to obtain IC₅₀ values.

Biological activity generated using the in vitro c-Met kinase assay I described herein:

| Example # | Activity |
|---|---|
| 1 | +++ |
| 2 | +++ |
| 3 | +++ |
| 4 | +++ |
| 5 | +++ |
| 6 | +++ |
| 7 | +++ |
| 8 | +++ |
| 9 | +++ |
| 10 | + |
| 11 | +++ |
| 12 | +++ |
| 13 | +++ |
| 14 | +++ |
| 15 | +++ |
| 16 | +++ |
| 17 | +++ |
| 18 | +++ |
| 19 | +++ |
| 20 | ++ |
| 21 | + |
| 22 | +++ |
| 23 | ++ |
| 24 | + |
| 25 | ++ |
| 26 | +++ |
| 27 | ++ |
| 28 | +++ |
| 29 | ++ |
| 30 | +++ |
| 31 | +++ |
| 32 | +++ |
| 33 | +++ |
| 34 | +++ |
| 35 | +++ |
| 36 | +++ |
| 37 | +++ |
| 38 | + |
| 39 | ++ |
| 40 | + |
| 41 | + |
| 42 | ++ |
| 43 | ++ |
| 44 | + |
| 45 | + |
| 46 | + |
| 47 | ++ |
| 48 | + |
| 49 | + |
| 50 | + |
| 51 | + |
| 52 | +++ |
| 53 | +++ |
| 54 | +++ |
| 55 | ++ |
| 56 | +++ |
| 57 | +++ |
| 58 | ++ |
| 59 | ++ |
| 60 | ++ |
| 61 | ++ |
| 62 | +++ |
| 63 | +++ |
| 64 | +++ |
| 65 | ++ |
| 66 | +++ |
| 67 | +++ |
| 68 | +++ |
| 69 | +++ |
| 70 | +++ |
| 71 | +++ |
| 72 | +++ |
| 73 | +++ |
| 74 | ++ |
| 75 | + |
| 76 | +++ |
| 77 | +++ |
| 78 | +++ |
| 79 | +++ |
| 80 | +++ |
| 81 | +++ |
| 82 | +++ |
| 83 | +++ |
| 84 | +++ |
| 85 | +++ |
| 86 | +++ |
| 87 | +++ |
| 88 | ++ |
| 89 | ++ |
| 90 | ++ |
| 91 | ++ |
| 92 | ++ |
| 93 | +++ |
| 94 | ++ |
| 95 | +++ |
| 96 | ++ |
| 97 | +++ |
| 98 | +++ |
| 99 | +++ |
| 100 | ++ |
| 101 | ++ |
| 102 | ++ |
| 103 | +++ |
| 104 | +++ |
| 105 | +++ |
| 106 | ++ |
| 107 | ++ |
| 108 | +++ |
| 109 | +++ |
| 110 | +++ |
| 111 | +++ |
| 112 | +++ |
| 113 | +++ |
| 114 | +++ |
| 115 | +++ |
| 116 | +++ |
| 117 | +++ |
| 118 | +++ |
| 119 | ++ |
| 120 | +++ |
| 121 | ++ |
| 122 | +++ |
| 123 | +++ |
| 124 | +++ |
| 125 | +++ |
| 126 | +++ |
| 127 | +++ |
| 128 | +++ |
| 129 | +++ |
| 130 | +++ |
| 131 | +++ |
| 132 | +++ |
| 133 | +++ |
| 134 | +++ |
| 135 | +++ |
| 136 | +++ |
| 137 | +++ |
| 138 | +++ |
| 139 | +++ |
| 140 | +++ |
| 141 | +++ |
| 142 | ++ |
| 143 | +++ |
| 144 | +++ |
| 145 | +++ |
| 146 | +++ |
| 147 | +++ |
| 148 | +++ |
| 149 | +++ |
| 150 | +++ |
| 151 | +++ |
| 152 | +++ |
| 153 | +++ |
| 154 | +++ |
| 155 | +++ |
| 156 | +++ |
| 157 | +++ |
| 158 | +++ |
| 159 | +++ |
| 160 | ++ |
| 161 | +++ |
| 162 | +++ |
| 163 | +++ |
| 164 | +++ |
| 165 | +++ |
| 166 | ++ |
| 167 | +++ |
| 168 | ++ |
| 169 | ++ |
| 170 | +++ |
| 171 | +++ |
| 172 | +++ |
| 173 | +++ |
| 174 | +++ |
| 175 | +++ |
| 176 | +++ |
| 177 | +++ |
| 178 | +++ |
| 179 | +++ |
| 180 | +++ |
| 181 | +++ |
| 182 | +++ |
| 183 | +++ |
| 184 | ++ |
| 185 | ++ |
| 186 | +++ |
| 187 | ++ |
| 188 | ++ |
| 189 | ++ |
| 190 | +++ |
| 191 | +++ |
| 192 | +++ |
| 193 | +++ |
| 194 | ++ |
| 195 | +++ |
| 196 | +++ |
| 197 | +++ |
| 198 | +++ |
| 199 | +++ |
| 200 | +++ |
| 201 | ++ |
| 202 | +++ |
| 203 | ++ |
| 204 | +++ |
| 205 | ++ |
| 206 | +++ |
| 207 | ++ |
| 208 | +++ |
| 209 | ++ |
| 210 | +++ |
| 211 | +++ |
| 212 | +++ |
| 213 | ++ |
| 214 | +++ |
| 215 | ++ |
| 216 | +++ |
| 217 | ++ |
| 218 | +++ |
| 219 | ++ |
| 220 | +++ |
| 221 | +++ |
| 222 | ++ |
| 223 | +++ |
| 224 | +++ |
| 225 | +++ |
| 226 | ++ |
| 227 | +++ |
| 228 | ++ |
| 229 | +++ |
| 230 | ++ |
| 231 | +++ |
| 232 | +++ |
| 233 | +++ |
| 234 | +++ |
| 235 | +++ |
| 236 | +++ |
| 237 | +++ |
| 238 | ++ |
| 239 | +++ |
| 240 | ++ |
| 241 | ++ |
| 242 | +++ |
| 243 | +++ |
| 244 | ++ |
| 245 | ++ |
| 246 | +++ |
| 247 | ++ |
| 248 | +++ |
| 249 | ++ |
| 250 | +++ |
| 251 | +++ |
| 252 | +++ |
| 253 | + |
| 254 | + |
| 255 | ++ |
| 256 | +++ |
| 257 | + |
| 258 | +++ |
| 259 | +++ |
| 260 | +++ |
| 261 | +++ |
| 262 | +++ |
| 263 | +++ |
| 264 | +++ |
| 265 | +++ |
| 266 | +++ |
| 267 | +++ |
| 268 | +++ |
| 269 | +++ |
| 270 | +++ |
| 271 | +++ |
| 272 | +++ |
| 273 | +++ |
| 274 | +++ |
| 275 | +++ |
| 276 | +++ |
| 277 | +++ |
| 278 | +++ |
| 279 | +++ |
| 280 | +++ |
| 281 | +++ |
| 282 | +++ |
| 283 | +++ |
| 284 | +++ |
| 285 | +++ |
| 286 | +++ |
| 287 | +++ |
| 288 | +++ |
| 289 | +++ |
| 290 | +++ |
| 291 | +++ |
| 292 | +++ |
| 293 | +++ |
| 294 | +++ |
| 295 | +++ |
| 296 | +++ |
| 297 | +++ |
| 298 | +++ |
| 299 | +++ |
| 300 | +++ |
| 301 | +++ |
| 302 | +++ |
| 303 | +++ |
| 304 | +++ |
| 305 | +++ |
| 306 | +++ |
| 307 | +++ |
| 308 | +++ |
| 309 | +++ |
| 310 | +++ |
| 311 | +++ |
| 312 | +++ |
| 313 | +++ |
| 314 | +++ |
| 315 | +++ |
| 316 | +++ |
| 317 | +++ |
| 318 | +++ |
| 319 | +++ |
| 320 | +++ |
| 321 | +++ |
| 322 | +++ |
| 323 | +++ |
| 324 | +++ |
| 325 | +++ |
| 326 | +++ |
| 327 | +++ |
| 328 | +++ |
| 329 | +++ |
| 330 | +++ |
| 331 | +++ |
| 332 | ++ |
| 333 | +++ |
| 334 | +++ |
| 335 | +++ |
| 336 | +++ |
| 337 | +++ |
| 338 | +++ |
| 339 | +++ |
| 340 | +++ |
| 341 | +++ |
| 342 | +++ |
| 343 | ++ |
| 344 | +++ |
| 345 | +++ |
| 346 | +++ |
| 347 | +++ |
| 348 | +++ |
| 349 | +++ |
| 350 | +++ |
| 351 | +++ |
| 352 | +++ |
| 353 | +++ |
| 354 | +++ |
| 355 | +++ |
| 356 | +++ |
| 357 | +++ |
| 358 | +++ |
| 359 | +++ |
| 360 | +++ |
| 361 | +++ |
| 362 | +++ |
| 363 | +++ |
| 364 | +++ |
| 365 | +++ |
| 366 | +++ |
| 367 | +++ |
| 368 | +++ |
| 369 | +++ |
| 370 | +++ |
| 371 | +++ |
| 372 | +++ |
| 373 | +++ |
| 374 | +++ |
| 375 | +++ |
| 376 | +++ |
| 377 | +++ |
| 378 | +++ |
| 379 | +++ |
| 380 | +++ |
| 381 | +++ |
| 382 | +++ |
| 383 | +++ |
| 384 | +++ |
| 385 | +++ |
| 386 | +++ |
| 387 | +++ |
| 388 | +++ |
| 389 | +++ |
| 390 | +++ |
| 391 | +++ |
| 392 | +++ |
| 393 | +++ |
| 394 | +++ |
| 395 | +++ |
| 396 | +++ |
| 397 | +++ |
| 398 | +++ |
| 399 | +++ |
| 400 | +++ |
| 401 | +++ |
| 402 | +++ |
| 403 | +++ |
| 404 | +++ |
| 405 | +++ |
| 406 | +++ |
| 407 | +++ |
| 408 | +++ |
| 409 | +++ |
| 410 | +++ |
| 411 | +++ |
| 412 | +++ |
| 413 | +++ |
| 414 | +++ |
| 415 | +++ |
| 416 | +++ |
| 417 | +++ |
| 418 | +++ |
| 419 | +++ |
| 420 | +++ |
| 421 | +++ |
| 422 | +++ |
| 423 | +++ |
| 424 | ++ |
| 425 | +++ |
| 426 | +++ |
| 427 | ++ |
| 428 | +++ |
| 429 | +++ |
| 430 | +++ |
| 431 | +++ |
| 432 | ++ |
| 433 | +++ |
| 434 | +++ |
| 435 | +++ |
| 436 | +++ |
| 437 | +++ |
| 438 | +++ |
| 439 | +++ |
| 440 | +++ |
| 441 | +++ |
| 442 | +++ |
| 443 | +++ |
| 444 | +++ |
| 445 | +++ |
| 446 | +++ |
| 447 | +++ |
| 448 | +++ |
| 449 | +++ |
| 450 | +++ |
| 451 | +++ |
| 452 | +++ |
| 453 | +++ |
| 454 | +++ |
| 455 | +++ |
| 456 | +++ |
| 457 | +++ |
| 458 | +++ |
| 459 | +++ |
| 460 | +++ |
| 461 | +++ |
| 462 | +++ |
| 463 | +++ |
| 464 | ++ |
| 465 | +++ |
| 466 | +++ |
| 467 | +++ |
| 468 | +++ |
| 469 | +++ |
| 470 | +++ |
| 471 | +++ |
| 472 | +++ |
| 473 | +++ |
| 474 | +++ |
| 475 | +++ |
| 476 | +++ |
| 477 | +++ |
| 478 | +++ |
| 479 | +++ |
| 480 | +++ |
| 481 | +++ |
| 482 | +++ |
| 483 | +++ |
| 484 | +++ |
| 485 | +++ |
| 486 | + |
| 487 | +++ |
| 488 | +++ |
| 489 | +++ |
| 490 | +++ |
| 491 | +++ |
| 492 | ++ |
| 493 | +++ |
| 494 | +++ |
| 495 | +++ |
| 496 | +++ |
| 497 | +++ |
| 498 | +++ |
| 499 | +++ |
| 500 | +++ |
| 501 | +++ |
| 502 | +++ |
| 503 | +++ |
| 504 | +++ |
| 505 | +++ |
| 506 | +++ |
| 507 | +++ |
| 508 | +++ |
| 509 | +++ |
| 510 | +++ |
| 511 | +++ |
| 512 | +++ |
| 513 | +++ |
| 514 | +++ |
| 515 | +++ |
| 516 | +++ |
| 517 | +++ |
| 518 | +++ |
| 519 | +++ |
| 520 | +++ |
| 521 | +++ |
| 522 | +++ |
| 523 | +++ |
| 524 | +++ |
| 525 | +++ |
| 526 | +++ |
| 527 | +++ |
| 528 | +++ |
| 529 | +++ |
| 530 | +++ |
| 531 | +++ |
| 532 | +++ |
| 533 | +++ |
| 534 | +++ |
| 535 | +++ |
| 536 | +++ |
| 537 | +++ |
| 538 | +++ |
| 539 | +++ |
| 540 | +++ |
| 541 | +++ |
| 542 | +++ |
| 543 | +++ |
| 544 | +++ |
| 545 | +++ |
| 546 | +++ |
| 547 | +++ |
| 548 | +++ |
| 549 | +++ |
| 550 | +++ |
| 551 | +++ |
| 552 | +++ |
| 553 | +++ |
| 554 | +++ |
| 555 | +++ |
| 556 | +++ |
| 557 | +++ |
| 558 | +++ |
| 559 | +++ |
| 560 | +++ |
| 561 | +++ |
| 562 | +++ |
| 563 | +++ |
| 564 | +++ |
| 565 | +++ |
| 566 | +++ |
| 567 | +++ |
| 568 | +++ |
| 569 | +++ |
| 576 | +++ |
| 571 | +++ |
| 572 | +++ |
| 573 | +++ |
| 574 | +++ |
| 575 | +++ |
| 576 | +++ |
| 577 | +++ |
| 578 | +++ |
| 579 | +++ |
| 580 | +++ |
| 581 | +++ |
| 582 | +++ |
| 583 | +++ |
| 584 | +++ |
| 585 | +++ |
| 586 | +++ |
| 587 | +++ |
| 588 | +++ |
| 589 | +++ |
| 590 | +++ |
| 591 | +++ |
| 592 | +++ |
| 593 | +++ |
| 594 | +++ |
| 595 | +++ |
| 596 | +++ |
| 597 | +++ |
| 598 | +++ |
| 599 | +++ |
| 600 | +++ |
| 601 | +++ |
| 602 | +++ |
| 603 | +++ |
| 604 | +++ |
| 605 | +++ |
| 606 | +++ |
| 607 | +++ |
| 608 | +++ |
| 609 | +++ |
| 610 | +++ |
| 611 | +++ |
| 612 | +++ |
| 613 | +++ |
| 614 | +++ |
| 615 | ++ |
| 616 | ++ |
| 617 | +++ |
| 618 | ++ |
| 619 | +++ |
| 620 | +++ |
| 621 | +++ |
| 622 | +++ |
| 623 | +++ |
| 624 | +++ |
| 625 | +++ |
| 626 | +++ |
| 627 | +++ |
| 628 | +++ |
| 629 | + |
| 630 | +++ |
| 631 | + |
| 632 | + |
| 633 | +++ |
| 634 | ++ |
| 635 | ++ |
| 636 | ++ |
| 637 | + |
| 638 | +++ |
| 639 | +++ |
| 640 | ++ |
| 641 | ++ |
| 642 | ++ |
| 643 | +++ |
| 644 | +++ |
| 645 | +++ |
| 646 | +++ |
| 647 | ++ |
| 648 | +++ |
| 649 | ++ |
| 650 | ++ |
| 651 | ++ |
| 652 | +++ |
| 653 | ++ |
| 654 | ++ |
| 655 | ++ |
| 656 | +++ |
| 657 | +++ |
| 658 | +++ |
| 659 | ++ |
| 660 | +++ |
| 661 | +++ |
| 662 | ++ |
| 663 | +++ |
| 664 | +++ |
| 665 | ++ |
| 666 | +++ |
| 667 | ++ |
| 668 | ++ |
| 669 | ++ |
| 670 | ++ |
| 671 | + |
| 672 | + |
| 673 | ++ |
| 674 | + |
| 675 | ++ |
| 676 | ++ |
| 677 | +++ |
| 678 | +++ |
| 679 | ++ |
| 680 | ++ |
| 681 | +++ |
| 682 | + |
| 683 | +++ |
| 684 | + |
| 685 | +++ |
| 686 | ++ |
| 687 | +++ |
| 688 | ++ |
| 689 | +++ |
| 690 | +++ |
| 691 | +++ |
| 692 | +++ |
| 693 | ++ |
| 694 | ++ |
| 695 | ++ |
| 696 | +++ |
| 697 | +++ |
| 698 | +++ |
| 699 | +++ |
| 700 | +++ |
| 701 | +++ |
| 702 | +++ |
| 703 | +++ |
| 704 | +++ |
| 705 | +++ |
| 706 | ++ |
| 707 | ++ |
| 708 | +++ |
| 709 | ++ |
| 710 | ++ |
| 711 | +++ |
| 712 | +++ |
| 713 | ++ |
| 714 | ++ |
| 715 | ++ |
| 716 | ++ |
| 717 | +++ |
| 718 | ++ |
| 719 | +++ |
| 720 | +++ |
| 721 | +++ |
| 722 | +++ |
| 723 | +++ |
| 724 | +++ |
| 725 | +++ |
| 726 | ++ |
| 727 | +++ |
| 728 | +++ |
| 729 | ++ |
| 730 | ++ |
| 731 | +++ |
| 732 | ++ |
| 733 | +++ |
| 734 | +++ |
| 735 | ++ |
| 736 | +++ |
| 737 | +++ |
| 738 | +++ |
| 739 | +++ |
| 740 | +++ |
| 741 | ++ |
| 742 | ++ |
| 743 | ++ |
| 744 | ++ |
| 745 | ++ |
| 746 | ++ |
| 747 | +++ |
| 748 | +++ |
| 749 | +++ |
| 750 | +++ |
| 751 | +++ |
| 752 | +++ |
| 753 | + |
| 754 | ++ |
| 755 | + |
| 756 | + |

+++ IC₅₀ < 100 nM
++ IC₅₀ = 100-1000 nM
+ IC₅₀ = 1000-3000 nM

What is claimed is:

1. A compound of the formula: 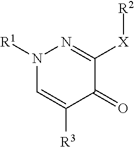
wherein X is O, S or CR⁴R^{4′};
R¹ is heteroaryl or aryl, wherein said heteroaryl and aryl groups are optionally substituted with one to three groups independently selected from the group consisting of halo, cyano, C₁₋₆ alkyl, (C₁₋₆ alkyl)R⁷, OR⁹, heterocyclyl(R⁷), aryl and heteroaryl(R⁵);
R² is heteroaryl or phenyl, wherein said heteroaryl group is optionally substituted with oxo, C₁₋₆ alkyl, NH(C═O)OR⁹ or OR⁹; and wherein said phenyl group is optionally substituted with one to two substituents independently selected from the group consisting of:
(1) halo,
(2) hydroxyl,
(3) cyano,
(4) heterocyclyl,
(5) heteroaryl, which is optionally substituted with one to two substituents independently selected from the group consisting of (C═O)OR⁹, NR⁵R⁹, NH(C═O)OR⁹, NH(C═O)R⁹, (C═O)NHR⁹, OR⁹ and R⁹,
(6) NH(C═O)OR⁹,
(7) NH(C═O)R⁹,
(8) NH(C═O)NHR⁹,
(9) (C═O)OR⁵, and
(10) C₁₋₃ alkyl(C═O)NHR⁵;
R³ is hydrogen, halo or C₁₋₃ alkyl;
R⁴ is hydrogen, halo or C₁₋₆ alkyl, wherein said alkyl is optionally substituted with hydroxyl or cyano;
R^{4′} is hydrogen, halo or C₁₋₆ alkyl, wherein said alkyl is optionally substituted with hydroxyl or cyano;
R⁵ is hydrogen or C₁₋₆ alkyl, wherein said alkyl is optionally substituted with hydroxyl;
R⁶ is hydrogen or C₁₋₆ alkyl, wherein said alkyl is optionally substituted with hydroxyl;
R⁷ is hydrogen, hydroxyl, C₁₋₆ alkyl, C₁₋₆ haloalkyl, heterocyclyl, OR⁹, heteroaryl(OR⁹), (C═O)R⁵, (C═O)OR⁵, (C═O)NR⁵R⁶, (C═O)heterocyclyl, (C═O)N(R⁵)heterocyclyl or NR⁵R⁶;
R⁸ is hydrogen, halo, cyano, hydroxyl, C₁₋₆ alkyl, (C═O)NR⁵R⁶ or NR⁵R⁶;
R⁹ is hydrogen, halo, C₁₋₆ alkyl, C₂₋₆ alkenyl, (C₂₋₆ alkenyl)OR⁵, (C₂₋₆ alkenyl)NR⁵R⁶, C₃₋₆ cycloalkyl, C₃₋₆ cycloalkyl(OR⁵), heterocyclyl (which is optionally substituted with one or two R⁸) or heteroaryl(R⁸), wherein said alkyl is optionally substituted with one to four groups independently selected from the group consisting of halo, hydroxyl, cyano, OR¹⁰, (C═O)NR⁵R⁶, (C═O)OR⁵, SO₂CH₃, NR⁵R¹⁰, C₃₋₈ cycloalkyl, heterocyclyl(which is optionally substituted with one or two)R¹⁰), heteroaryl(R¹⁰), (aryl)OR⁵, phenyl and phenyl(O-benzyl);
R¹⁰ is hydrogen, halo, oxo, C₁₋₆ alkyl, (C₁₋₆ alkyl)OR⁵, C₁₋₆ haloalkyl, C₃₋₈ cycloalkyl, aryl and (C═O)OR⁵;
or a pharmaceutically acceptable salt thereof.

2. The compound of claim 1 wherein R¹ is heteroaryl, wherein said heteroaryl group is optionally substituted with one to three groups independently selected from the group consisting of halo, cyano, C₁₋₆ alkyl, (C₁₋₆ alkyl)R⁷, OR⁹, heterocyclyl(R⁷), aryl and heteroaryl(R⁵); or a pharmaceutically acceptable salt thereof.

3. The compound of claim 2 wherein R¹ is heteroaryl, wherein said heteroaryl group is optionally substituted with C₁₋₆ alkyl, or a pharmaceutically acceptable salt thereof.

4. The compound of claim 2 wherein X is CR⁴R^{4′}, R⁴ is hydrogen, R^{4′} is hydrogen, or a pharmaceutically acceptable salt thereof.

5. The compound of claim 1 wherein R² is phenyl, wherein said phenyl group is optionally substituted with one to two substituents independently selected from the group consisting of:
(1) halo,
(2) hydroxyl,
(3) cyano,
(4) heterocyclyl,
(5) heteroaryl, which is optionally substituted with one to two substituents independently selected from the group consisting of (C═O)OR⁹, NR⁵R⁹, NH(C═O)OR⁹, NH(C═O)R⁹, (C═O)NHR⁹, OR⁹ and R⁹,
(6) NH(C═O)OR⁹,
(7) NH(C═O)R⁹,
(8) NH(C═O)NHR⁹,
(9) (C═O)OR⁵, and
(10) C₁₋₃ alkyl(C═O)NHR⁵;
or a pharmaceutically acceptable salt thereof.

6. The compound of claim 5 wherein R² is phenyl, wherein said phenyl group is substituted with heteroaryl, which is optionally substituted with OR⁹ or R⁹, or a pharmaceutically acceptable salt thereof.

7. The compound of claim 1 selected from:
ethyl(3-{[1-(1-methyl-1H-pyrazol-4-yl)-4-oxo-1,4-dihydropyridazin-3-yl]methyl}phenyl)carbamate;
ethyl(3-{[4-oxo-1-(3,4,5-trifluorophenyl)-1,4-dihydropyridazin-3-yl]methyl}phenyl)carbamate;
3-[3-(5-ethoxypyrimidin-2-yl)benzyl]-1-(1-methyl-1H-pyrazol-4-yl)pyridazin-4(1H)-one;
3-{3-[5-(2-methoxyethoxy)pyrimidin-2-yl]benzyl}-1-(1-methyl-1H-pyrazol-4-yl)pyridazin-4(1H)-one;
3-[3-(5-methoxypyrimidin-2-yl)benzyl]-1-(1-methyl-1H-pyrazol-4-yl)pyridazin-4(1H)-one;
3-fluoro-5-[3-{3-[5-(2-hydroxy-2-methylpropoxy)pyrimidin-2-yl]benzyl}-4-oxopyridazin-1(4H)-yl]benzonitrile;
2-methylpropyl (3-{[4-oxo-1-(3,4,5-trifluorophenyl)-1,4-dihydropyridazin-3-yl]methyl}phenyl)carbamate;
propyl (3-{[4-oxo-1-(3,4,5-trifluorophenyl)-1,4-dihydropyridazin-3-yl]methyl}phenyl)carbamate;
3-[3-(5-methoxypyrimidin-2-yl)benzyl]-1-(3,4,5-trifluorophenyl)pyridazin-4(1H)-one;
3-([1,2,4]triazolo[1,5-a]pyridin-6-ylmethyl)-1-(3,4,5-trifluorophenyl)pyridazin-4(1H)-one;
ethyl(3-{[1-(3,5-difluorophenyl)-4-oxo-1,4-dihydropyridazin-3-yl]methyl}phenyl)carbamate;
propyl (3-{[1-(3-bromophenyl)-4-oxo-1,4-dihydropyridazin-3-yl]methyl}phenyl)carbamate;
ethyl(3-{[1-(3-bromophenyl)-4-oxo-1,4-dihydropyridazin-3-yl]methyl}phenyl)carbamate;
propyl (3-{[1-(4-bromophenyl)-4-oxo-1,4-dihydropyridazin-3-yl]methyl}phenyl)carbamate;
methyl 2-(3-{[1-(1-methyl-1H-pyrazol-4-yl)-4-oxo-1,4-dihydropyridazin-3-yl]methyl}phenyl)pyrimidine-5-carboxylate;
propyl (3-{[1-(4-bromo-3,5-difluorophenyl)-4-oxo-1,4-dihydropyridazin-3-yl]methyl}phenyl)carbamate;
2-methylpropyl (3-{[1-(3,5-difluorophenyl)-4-oxo-1,4-dihydropyridazin-3-yl]methyl}phenyl)carbamate;
ethyl(3-{[1-(4-bromophenyl)-4-oxo-1,4-dihydropyridazin-3-yl]methyl}phenyl)carbamate;
1-(1-methyl-1H-pyrazol-4-yl)-3-[3-(5-methyl-1,3-thiazol-2-yl)benzyl]pyridazin-4(1H)-one;
1-(1-methyl-1H-pyrazol-4-yl)-3-[3-(1,3-thiazol-2-yl)benzyl]pyridazin-4(1H)-one;
1-(1-methyl-1H-pyrazol-4-yl)-3-[3-(1H-pyrazol-1-yl)benzyl]pyridazin-4(1H)-one;
2-morpholin-4-ylethyl(3-{[1-(3,4-difluorophenyl)-4-oxo-1,4-dihydropyridazin-3-yl]methyl}phenyl)carbamate;
5-{[1-(1-methyl-1H-pyrazol-4-yl)-4-oxo-1,4-dihydropyridazin-3-yl]methyl}-1,3-dihydro-2H-benzimidazol-2-one;
5-{[1-(1-methyl-1H-pyrazol-4-yl)-4-oxo-1,4-dihydropyridazin-3-yl]methyl}-1,3-benzoxazol-2(3H)-one;
ethyl(3-{[1-(1-methyl-1H-pyrazol-3-yl)-4-oxo-1,4-dihydropyridazin-3-yl]methyl}phenyl)carbamate;
2-methylpropyl (3-{[1-(1-methyl-1H-pyrazol-3-yl)-4-oxo-1,4-dihydropyridazin-3-yl]methyl}phenyl)carbamate;
propyl (3-{[1-(1-methyl-1H-pyrazol-3-yl)-4-oxo-1,4-dihydropyridazin-3-yl]methyl}phenyl)carbamate;
propyl {3-[(4-oxo-1-pyridin-3-yl-1,4-dihydropyridazin-3-yl)methyl]phenyl}carbamate;
1-pyridin-3-yl-3-(3-pyrimidin-2-ylbenzyl)pyridazin-4(1H)-one;
propyl {3-[(4-oxo-1-pyridin-4-yl-1,4-dihydropyridazin-3-yl)methyl]phenyl}carbamate;
ethyl(3-{[1-(6-methoxypyridin-3-yl)-4-oxo-1,4-dihydropyridazin-3-yl]methyl}phenyl)carbamate;
propyl (3-{[1-(6-methoxypyridin-3-yl)-4-oxo-1,4-dihydropyridazin-3-yl]methyl}phenyl)carbamate;
1-(6-methoxypyridin-3-yl)-3-[3-(5-methoxypyrimidin-2-yl)benzyl]pyridazin-4(1H)-one;
ethyl(3-{[1-(5-fluoropyridin-3-yl)-4-oxo-1,4-dihydropyridazin-3-yl]methyl}phenyl)carbamate;
propyl (3-{[1-(5-fluoropyridin-3-yl)-4-oxo-1,4-dihydropyridazin-3-yl]methyl}phenyl)carbamate;
1-(5-fluoropyridin-3-yl)-3-[3-(5-methoxypyrimidin-2-yl)benzyl]pyridazin-4(1H)-one;
3-[3-(5-methoxypyrimidin-2-yl)benzyl]-1-(5-methylpyridin-3-yl)pyridazin-4(1H)-one;
1-(1-methyl-1H-pyrazol-4-yl)-3-[3-(pyrazin-2-yl)benzyl]pyridazin-4(1H)-one;
1-(1-methyl-1H-pyrazol-4-yl)-3-[3-(1-methyl-1H-pyrazol-3-yl)benzyl]pyridazin-4(1H)-one;
1-(1-methyl-1H-pyrazol-4-yl)-3-[3-(2-methylpyrimidin-4-yl)benzyl]pyridazin-4(1H)-one;
3-[3-(3-methyl-1,2,4-oxadiazol-5-yl)benzyl]-1-(1-methyl-1H-pyrazol-4-yl)pyridazin-4(1H)-one;
3-[3-(5-methyl-1,2,4-oxadiazol-3-yl)benzyl]-1-(1-methyl-1H-pyrazol-4-yl)pyridazin-4(1H)-one;
1-(4-chlorophenyl)-3-(quinolin-6-ylmethyl)pyridazin-4(1H)-one
1-(1-methyl-1H-pyrazol-4-yl)-3-[(2-methylquinolin-5-yl)methyl]pyridazin-4(1H)-one;
4-{[1-(1-methyl-1H-pyrazol-4-yl)-4-oxo-1,4-dihydropyridazin-3-yl]methyl}-2,3-dihydro-1H-isoindol-1-one;
3-(imidazo[1,2-a]pyridin-6-ylmethyl)-1-(1-methyl-1H-pyrazol-4-yl)pyridazin-4(1H)-one;
ethyl 2-fluoro-3-{[1-(1-methyl-1H-pyrazol-4-yl)-4-oxo-1,4-dihydropyridazin-3-yl]methyl}benzoate;
2-(3-{[1-(1-methyl-1H-pyrazol-4-yl)-4-oxo-1,4-dihydropyridazin-3-yl]methyl}phenyl)acetamide;
3-[(2-methyl-2H-indazol-5-yl)methyl]-1-(1-methyl-1H-pyrazol-4-yl)pyridazin-4(1H)-one;
3-(1H-indazol-4-ylmethyl)-1-(1-methyl-1H-pyrazol-4-yl)pyridazin-4(1H)-one;
3-(1-benzofuran-5-ylmethyl)-1-(1-methyl-1H-pyrazol-4-yl)pyridazin-4(1H)-one;
propyl (3-{[1-(1-methyl-1H-pyrazol-4-yl)-4-oxo-1,4-dihydropyridazin-3-yl]methyl}phenyl)carbamate;
2-methylpropyl (3-{[1-(1-methyl-1H-pyrazol-4-yl)-4-oxo-1,4-dihydropyridazin-3-yl]methyl}phenyl)carbamate;
2-methoxyethyl(3-{[1-(1-methyl-1H-pyrazol-4-yl)-4-oxo-1,4-dihydropyridazin-3-yl]methyl}phenyl)carbamate;
1-(1-methyl-1H-pyrazol-4-yl)-3-(quinolin-6-ylmethyl)pyridazin-4(1H)-one;
propyl (3-{[1-(2,6-dichloropyridin-4-yl)-4-oxo-1,4-dihydropyridazin-3-yl]methyl}phenyl)carbamate;
1-(1-methyl-1H-pyrazol-4-yl)-3-[3-(1-propyl-1H-1,2,4-triazol-3-yl)benzyl]pyridazin-4(1H)-one;
1-(1-methyl-1H-pyrazol-4-yl)-3-[3-(2-methyl-2H-tetrazol-5-yl)benzyl]pyridazin-4(1H)-one;
3-[(3-ethoxyquinolin-6-yl)methyl]-1-(1-methyl-1H-pyrazol-4-yl)pyridazin-4(1H)-one;
1-(1-methyl-1H-pyrazol-4-yl)-3-{[3-(2-morpholin-4-ylethoxy)quinolin-6-yl]methyl}pyridazin-4(1H)-one;
3-{[3-(2-methoxyethoxy)quinolin-6-yl]methyl}-1-(1-methyl-1H-pyrazol-4-yl)pyridazin-4(1H)-one;
3-({3-[(3-methyloxetan-3-yl)methoxy]quinolin-6-yl}methyl)-1-(1-methyl-1H-pyrazol-4-yl)pyridazin-4(1H)-one;
1-(1-methyl-1H-pyrazol-4-yl)-3-[(3-propoxyquinolin-6-yl)methyl]pyridazin-4(1H)-one;
rac-1-(1-methyl-1H-pyrazol-4-yl)-3-{[3-(tetrahydrofuran-3-ylmethoxy)quinolin-6-yl]methyl}pyridazin-4(1H)-one;
3-[(3-ethoxyquinolin-6-yl)methyl]-1-(3,4,5-trifluorophenyl)pyridazin-4(1H)-one;
3-{3-[1-(2-methoxyethyl)-1H-1,2,4-triazol-3-yl]benzyl}-1-(1-methyl-1H-pyrazol-4-yl)pyridazin-4(1H)-one;
3-{3-[5-(benzyloxy)pyrimidin-2-yl]benzyl}-1-(1-methyl-1H-pyrazol-4-yl)pyridazin-4(1H)-one;
2-methylpropyl (3-{[1-(4-bromophenyl)-4-oxo-1,4-dihydropyridazin-3-yl]methyl}phenyl)carbamate;
2-methylpropyl (3-{[1-(3-bromophenyl)-4-oxo-1,4-dihydropyridazin-3-yl]methyl}phenyl)carbamate;
2-methoxyethyl(3-{[1-(1-ethyl-1H-pyrazol-4-yl)-4-oxo-1,4-dihydropyridazin-3-yl]methyl}phenyl)carbamate;
3-{3-[5-(benzyloxy)pyrimidin-2-yl]benzyl}-1-(1-ethyl-1H-pyrazol-4-yl)pyridazin-4(1H)-one;
3-[3-[3-(5-ethoxypyrimidin-2-yl)benzyl]-4-oxopyridazin-1(4H)-yl]benzonitrile;
2-methoxyethyl(3-{[1-(3-cyano-5-fluorophenyl)-4-oxo-1,4-dihydropyridazin-3-yl]methyl}phenyl)carbamate;
3-(isoquinolin-6-ylmethyl)-1-(1-methyl-1H-pyrazol-4-yl)pyridazin-4(1H)-one;
1-(1-methyl-1H-pyrazol-4-yl)-3-[3-(5-methyl-1,3,4-thiadiazol-2-yl)benzyl]pyridazin-4(1H)-one;
3-[3-(1-butyl-1H-1,2,4-triazol-3-yl)benzyl]-1-(1-methyl-1H-pyrazol-4-yl)pyridazin-4(1H)-one;
3-{3-[1-(3-methoxypropyl)-1H-1,2,4-triazol-3-yl]benzyl}-1-(1-methyl-1H-pyrazol-4-yl)pyridazin-4(1H)-one;
3-{3-[1-(3-methylbutyl)-1H-1,2,4-triazol-3-yl]benzyl}-1-(1-methyl-1H-pyrazol-4-yl)pyridazin-4(1H)-one;
rac-1-(1-methyl-1H-pyrazol-4-yl)-3-{3-[1-(tetrahydrofuran-3-ylmethyl)-1H-1,2,4-triazol-3-yl]benzyl}pyridazin-4(1H)-one;
1-(3,4-difluorophenyl)-3-[3-(1-propyl-1H-1,2,4-triazol-3-yl)benzyl]pyridazin-4(1H)-one;
3-fluoro-5-{4-oxo-3-[3-(1-propyl-1H-1,2,4-triazol-3-yl)benzyl]pyridazin-1(4H)-yl}benzonitrile;
1-(3,4-difluorophenyl)-3-[3-(1-ethyl-1H-1,2,4-triazol-3-yl)benzyl]pyridazin-4(1H)-one;
3-{3-[3-(1-ethyl-1H-1,2,4-triazol-3-yl)benzyl]-4-oxopyridazin-1(4H)-yl}-5-fluorobenzonitrile;
3-{3-[3-(1-ethyl-1H-1,2,4-triazol-3-yl)benzyl]-4-oxopyridazin-1(4H)-yl}benzonitrile;
3-{4-oxo-3-[3-(1-propyl-1H-1,2,4-triazol-3-yl)benzyl]pyridazin-1(4H)-yl}benzonitrile;
1-(1-ethyl-1H-pyrazol-4-yl)-3-[3-(1-ethyl-1H-1,2,4-triazol-3-yl)benzyl]pyridazin-4(1H)-one;
1-(1-ethyl-1H-pyrazol-4-yl)-3-[3-(1-propyl-1H-1,2,4-triazol-3-yl)benzyl]pyridazin-4(1H)-one;
1-{1-[2-(benzyloxy)ethyl]-1H-pyrazol-4-yl}-3-[3-(1-ethyl-1H-1,2,4-triazol-3-yl)benzyl]pyridazin-4(1H)-one;
1-{1-[2-(benzyloxy)ethyl]-1H-pyrazol-4-yl}-3-[3-(1-propyl-1H-1,2,4-triazol-3-yl)benzyl]pyridazin-4(1H)-one;
2-methylpropyl[3-({4-oxo-1-[1-(tetrahydro-2H-pyran-4-yl)-1H-pyrazol-4-yl]-1,4-dihydropyridazin-3-yl}methyl)phenyl]carbamate;
ethyl [3-({4-oxo-1-[1-(tetrahydro-2H-pyran-4-yl)-1H-pyrazol-4-yl]-1,4-dihydropyridazin-3-yl}methyl)phenyl]carbamate;
ethyl [3-({1-[1-(2-methylpropyl)-1H-pyrazol-4-yl]-4-oxo-1,4-dihydropyridazin-3-yl}methyl)phenyl]carbamate;
2-methylpropyl[3-({4-oxo-1-[1-(2,2,2-trifluoroethyl)-1H-pyrazol-4-yl]-1,4-dihydropyridazin-3-yl}methyl)phenyl]carbamate;
2-methylpropyl[3-({1-[1-(2-methylpropyl)-1H-pyrazol-4-yl]-4-oxo-1,4-dihydropyridazin-3-yl}methyl)phenyl]carbamate;
3-[3-(5-methoxypyrimidin-2-yl)benzyl]-1-[1-(2-methylpropyl)-1H-pyrazol-4-yl]pyridazin-4(1H)-one;
2-morpholin-4-ylethyl [3-({1-[1-(2-methylpropyl)-1H-pyrazol-4-yl]-4-oxo-1,4-dihydropyridazin-3-yl}methyl)phenyl]carbamate;
rac-ethyl [3-({4-oxo-1-[1-(tetrahydrofuran-3-yl)-1H-pyrazol-4-yl]-1,4-dihydropyridazin-3-yl}methyl)phenyl]carbamate;
rac-propyl[3-({4-oxo-1-[1-(tetrahydrofuran-3-yl)-1H-pyrazol-4-yl]-1,4-dihydropyridazin-3-yl}methyl)phenyl]carbamate;
rac-2-methylpropyl[3-({4-oxo-1-[1-(tetrahydrofuran-3-yl)-1H-pyrazol-4-yl]-1,4-dihydropyridazin-3-yl}methyl)phenyl]carbamate;
ethyl [3-({4-oxo-1-[1-(tetrahydro-2H-pyran-4-ylmethyl)-1H-pyrazol-4-yl]-1,4-dihydropyridazin-3-yl}methyl)phenyl]carbamate;
propyl[3-({4-oxo-1-[1-(tetrahydro-2H-pyran-4-ylmethyl)-1H-pyrazol-4-yl]-1,4-dihydropyridazin-3-yl}methyl)phenyl]carbamate;
2-methylpropyl[3-({4-oxo-1-[1-(tetrahydro-2H-pyran-4-ylmethyl)-1H-pyrazol-4-yl]-1,4-dihydropyridazin-3-yl}methyl)phenyl]carbamate;
ethyl [3-({1-[1-(1-methylethyl)-1H-pyrazol-4-yl]-4-oxo-1,4-dihydropyridazin-3-yl}methyl)phenyl]carbamate;
propyl[3-({1-[1-(1-methylethyl)-1H-pyrazol-4-yl]-4-oxo-1,4-dihydropyridazin-3-yl}methyl)phenyl]carbamate;
2-methylpropyl[3-({1-[1-(1-methylethyl)-1H-pyrazol-4-yl]-4-oxo-1,4-dihydropyridazin-3-yl}methyl)phenyl]carbamate;
ethyl [3-({1-[1-(2-methoxyethyl)-1H-pyrazol-4-yl]-4-oxo-1,4-dihydropyridazin-3-yl}methyl)phenyl]carbamate;
propyl[3-({1-[1-(2-methoxyethyl)-1H-pyrazol-4-yl]-4-oxo-1,4-dihydropyridazin-3-yl}methyl)phenyl]carbamate;
2-methylpropyl[3-({1-[1-(2-methoxyethyl)-1H-pyrazol-4-yl]-4-oxo-1,4-dihydropyridazin-3-yl}methyl)phenyl]carbamate;
ethyl(3-{[1-(1-ethyl-1H-pyrazol-4-yl)-4-oxo-1,4-dihydropyridazin-3-yl]methyl}phenyl)carbamate;
2-methylpropyl (3-{[1-(1-ethyl-1H-pyrazol-4-yl)-4-oxo-1,4-dihydropyridazin-3-yl]methyl}phenyl)carbamate;
ethyl [3-({4-oxo-1-[1-(2,2,2-trifluoroethyl)-1H-pyrazol-4-yl]-1,4-dihydropyridazin-3-yl}methyl)phenyl]carbamate;
ethyl(3-{[4-oxo-1-(1-propyl-1H-pyrazol-4-yl)-1,4-dihydropyridazin-3-yl]methyl}phenyl)carbamate;
2-methylpropyl (3-{[4-oxo-1-(1-propyl-1H-pyrazol-4-yl)-1,4-dihydropyridazin-3-yl]methyl}phenyl)carbamate;
3-[3-(5-methoxypyrimidin-2-yl)benzyl]-1-(1-propyl-1H-pyrazol-4-yl)pyridazin-4(1H)-one;
2-morpholin-4-ylethyl(3-{[1-(1-ethyl-1H-pyrazol-4-yl)-4-oxo-1,4-dihydropyridazin-3-yl]methyl}phenyl)carbamate;
2-morpholin-4-ylethyl [3-({4-oxo-1-[1-(2,2,2-trifluoroethyl)-1H-pyrazol-4-yl]-1,4-dihydropyridazin-3-yl}methyl)phenyl]carbamate;
2-morpholin-4-ylethyl(3-{[4-oxo-1-(1-propyl-1H-pyrazol-4-yl)-1,4-dihydropyridazin-3-yl]methyl}phenyl)carbamate;
3-[5-(5-ethoxypyrimidin-2-yl)-2-fluorobenzyl]-1-(1-methyl-1H-pyrazol-4-yl)pyridazin-4(1H)-one;
3-[3-(5-ethoxypyrimidin-2-yl)-4-fluorobenzyl]-1-(1-methyl-1H-pyrazol-4-yl)pyridazin-4(1H)-one;
3-[3-(5-ethoxypyrimidin-2-yl)-5-fluorobenzyl]-1-(1-methyl-1H-pyrazol-4-yl)pyridazin-4(1H)-one;
3-[3-(5-ethoxypyrimidin-2-yl)-2-fluorobenzyl]-1-(1-methyl-1H-pyrazol-4-yl)pyridazin-4(1H)-one;
3-(3-{5-[(trans-4-hydroxy-4-methylcyclohexyl)oxy]pyrimidin-2-yl}benzyl)-1-(1-methyl-1H-pyrazol-4-yl)pyridazin-4(1H)-one;
3-{3-[5-(1,4-dioxaspiro[4.5]dec-8-yloxy)pyrimidin-2-yl]benzyl}-1-(1-methyl-1H-pyrazol-4-yl)pyridazin-4(1H)-one;
3-{3-[5-(benzyloxy)pyrimidin-2-yl]benzyl}-1-(3,4-difluorophenyl)pyridazin-4(1H)-one;
3-{3-[5-(benzyloxy)pyrimidin-2-yl]benzyl}-1-(3,5-difluorophenyl)pyridazin-4(1H)-one;
3-chloro-5-{3-[3-(5-methoxypyrimidin-2-yl)benzyl]-4-oxopyridazin-1(4H)-yl}benzonitrile;
2-methoxyethyl(3-{[1-(3-chloro-5-cyanophenyl)-4-oxo-1,4-dihydropyridazin-3-yl]methyl}phenyl)carbamate;
3-fluoro-5-{3-[3-(5-methoxypyrimidin-2-yl)benzyl]-4-oxopyridazin-1(4H)-yl}benzonitrile;
2-methoxyethyl(3-{[1-(5-cyanopyridin-3-yl)-4-oxo-1,4-dihydropyridazin-3-yl]methyl}phenyl)carbamate;
rac-tetrahydrofuran-3-ylmethyl(3-{[1-(5-cyanopyridin-3-yl)-4-oxo-1,4-dihydropyridazin-3-yl]methyl}phenyl)carbamate;
rac-tetrahydrofuran-3-ylmethyl(3-{[1-(3-cyano-5-fluorophenyl)-4-oxo-1,4-dihydropyridazin-3-yl]methyl}phenyl)carbamate;
5-[3-{3-[5-(2-methoxyethoxy)pyrimidin-2-yl]benzyl}-4-oxopyridazin-1(4H)-yl]pyridine-3-carbonitrile;
3-{3-[3-(5-ethoxypyrimidin-2-yl)benzyl]-4-oxopyridazin-1(4H)-yl}-5-fluorobenzonitrile;
3-fluoro-5-[3-{3-[5-(2-methoxyethoxy)pyrimidin-2-yl]benzyl}-4-oxopyridazin-1(4H)-yl]benzonitrile;
ethyl(3-{[1-(3-cyanophenyl)-4-oxo-1,4-dihydropyridazin-3-yl]methyl}phenyl)carbamate;
5-{3-[3-(5-ethoxypyrimidin-2-yl)benzyl]-4-oxopyridazin-1(4H)-yl}pyridine-3-carbonitrile;
4-{3-[3-(5-ethoxypyrimidin-2-yl)benzyl]-4-oxopyridazin-1(4H)-yl}benzonitrile;
4-[3-{3-[5-(2-methoxyethoxy)pyrimidin-2-yl]benzyl}-4-oxopyridazin-1(4H)-yl]benzonitrile;
rac-tetrahydrofuran-3-ylmethyl(3-{[1-(4-cyanophenyl)-4-oxo-1,4-dihydropyridazin-3-yl]methyl}phenyl)carbamate;
3-chloro-5-{3-[3-(5-ethoxypyrimidin-2-yl)benzyl]-4-oxopyridazin-1(4H)-yl}benzonitrile;
rac-tetrahydrofuran-3-ylmethyl(3-{[1-(3-chloro-5-cyanophenyl)-4-oxo-1,4-dihydropyridazin-3-yl]methyl}phenyl)carbamate;
3-[(4-methoxyquinolin-6-yl)methyl]-1-(1-methyl-1H-pyrazol-4-yl)pyridazin-4(1H)-one;
1-(3-bromophenyl)-3-[3-(5-ethoxypyrimidin-2-yl)benzyl]pyridazin-4(1H)-one;
2-fluoro-4-[3-{3-[5-(2-methoxyethoxy)pyrimidin-2-yl]benzyl}-4-oxopyridazin-1(4H)-yl]benzonitrile;
1-(4-bromo-3-fluorophenyl)-3-[3-(5-ethoxypyrimidin-2-yl)benzyl]pyridazin-4(1H)-one;
1-(3,5-difluorophenyl)-3-[3-(5-ethoxypyrimidin-2-yl)benzyl]pyridazin-4(1H)-one;
1-(4-bromo-3,5-difluorophenyl)-3-[3-(5-ethoxypyrimidin-2-yl)benzyl]pyridazin-4(1H)-one;
4-{3-[3-(5-ethoxypyrimidin-2-yl)benzyl]-4-oxopyridazin-1(4H)-yl}-2-fluorobenzonitrile;
1-(3,5-difluorophenyl)-3-{3-[5-(2-methoxyethoxy)pyrimidin-2-yl]benzyl}pyridazin-4(1H)-one;
3-[3-(5-ethoxypyrimidin-2-yl)benzyl]-1-(1-ethyl-1H-pyrazol-4-yl)pyridazin-4(1H)-one;
1-(1-ethyl-1H-pyrazol-4-yl)-3-{3-[5-(2-methoxyethoxy)pyrimidin-2-yl]benzyl}pyridazin-4(1H)-one;
1-(4-chloro-3-fluorophenyl)-3-[3-(5-ethoxypyrimidin-2-yl)benzyl]pyridazin-4(1H)-one;
1-(4-chloro-3-fluorophenyl)-3-{3-[5-(2-methoxyethoxy)pyrimidin-2-yl]benzyl}pyridazin-4(1H)-one;
1-(1-methyl-1H-pyrazol-4-yl)-3-{3-[5-(1-methyl-1H-pyrazol-4-yl)pyrimidin-2-yl]benzyl}pyridazin-4(1H)-one;
3-fluoro-5-[3-{3-[5-(1-methyl-1H-pyrazol-4-yl)pyrimidin-2-yl]benzyl}-4-oxopyridazin-1(4H)-yl]benzonitrile;
1-(3,4-difluorophenyl)-3-{3-[5-(1-methyl-1H-pyrazol-4-yl)pyrimidin-2-yl]benzyl}pyridazin-4(1H)-one;
1-(1-ethyl-1H-pyrazol-4-yl)-3-{3-[5-(1-methyl-1H-pyrazol-4-yl)pyrimidin-2-yl]benzyl}pyridazin-4(1H)-one;
3-chloro-5-[3-{3-[5-(1-methyl-1H-pyrazol-4-yl)pyrimidin-2-yl]benzyl}-4-oxopyridazin-1(4H)-yl]benzonitrile;
tert-butyl [2-(3-{[1-(1-methyl-1H-pyrazol-4-yl)-4-oxo-1,4-dihydropyridazin-3-yl]methyl}phenyl)pyrimidin-5-yl]carbamate;
3-[(3-ethoxyquinolin-6-yl)methyl]-1-(1-ethyl-1H-pyrazol-4-yl)pyridazin-4(1H)-one;
3-{3-[5-(methoxymethyl)pyrimidin-2-yl]benzyl}-1-(1-methyl-1H-pyrazol-4-yl)pyridazin-4(1H)-one;
3-{3-[5-(ethoxymethyl)pyrimidin-2-yl]benzyl}-1-(1-methyl-1H-pyrazol-4-yl)pyridazin-4(1H)-one;
1-(1-ethyl-1H-pyrazol-4-yl)-3-{3-[5-(methoxymethyl)pyrimidin-2-yl]benzyl}pyridazin-4(1H)-one;
1-(1-methyl-1H-pyrazol-4-yl)-3-{3-[5-(oxetan-3-yloxy)pyrimidin-2-yl]benzyl}pyridazin-4(1H)-one;
1-(1-ethyl-1H-pyrazol-4-yl)-3-{3-[5-(oxetan-3-yloxy)pyrimidin-2-yl]benzyl}pyridazin-4(1H)-one;
rac-3-{3-[5-(1-methoxyethyl)pyrimidin-2-yl]benzyl}-1-(1-methyl-1H-pyrazol-4-yl)pyridazin-4(1H)-one;
3-{3-[5-(1-hydroxy-1-methylethyl)pyrimidin-2-yl]benzyl}-1-(1-methyl-1H-pyrazol-4-yl)pyridazin-4(1H)-one;
3-{3-[5-(1-methoxy-1-methylethyl)pyrimidin-2-yl]benzyl}-1-(1-methyl-1H-pyrazol-4-yl)pyridazin-4(1H)-one;
3-{3-[5-(1-ethoxy-1-methylethyl)pyrimidin-2-yl]benzyl}-1-(1-methyl-1H-pyrazol-4-yl)pyridazin-4(1H)-one;
3-fluoro-5-[3-(3-{5-[(1-methylpiperidin-4-yl)methoxy]pyrimidin-2-yl}benzyl)-4-oxopyridazin-1(4H)-yl]benzonitrile;
3-[3-(3-{5-[(1-methylpiperidin-4-yl)methoxy]pyrimidin-2-yl}benzyl)-4-oxopyridazin-1(4H)-yl]benzonitrile;
1-(1-ethyl-1H-pyrazol-4-yl)-3-(3-{5-[(1-methylpiperidin-4-yl)methoxy]pyrimidin-2-yl}benzyl)pyridazin-4(1H)-one;
1-(3,5-difluorophenyl)-3-(3-{5-[(1-methylpiperidin-4-yl)methoxy]pyrimidin-2-yl}benzyl)pyridazin-4(1H)-one;
rac-3-{3-[5-(2,5-dihydrofuran-2-yl)pyrimidin-2-yl]benzyl}-1-(1-methyl-1H-pyrazol-4-yl)pyridazin-4(1H)-one;
3-{3-[5-(2,5-dihydrofuran-3-yl)pyrimidin-2-yl]benzyl}-1-(1-methyl-1H-pyrazol-4-yl)pyridazin-4(1H)-one;
3-[3-(4-butyl-5-ethoxypyrimidin-2-yl)benzyl]-1-(1-methyl-1H-pyrazol-4-yl)pyridazin-4(1H)-one;
3-[3-(5-ethoxy-4-methylpyrimidin-2-yl)benzyl]-1-(1-methyl-1H-pyrazol-4-yl)pyridazin-4(1H)-one;
3-[3-(5-ethoxy-4-ethylpyrimidin-2-yl)benzyl]-1-(1-methyl-1H-pyrazol-4-yl)pyridazin-4(1H)-one;
methyl 4-{4-[3-(3-{[(2-methylpropoxy)carbonyl]amino}benzyl)-4-oxopyridazin-1(4H)-yl]-1H-pyrazol-1-yl}butanoate;
4-{4-[3-(3-{[(2-methylpropoxy)carbonyl]amino}benzyl)-4-oxopyridazin-1(4H)-yl]-1H-pyrazol-1-yl}butanoic acid;
{4-[3-(3-{[(2-methylpropoxy)carbonyl]amino}benzyl)-4-oxopyridazin-1(4H)-yl]-1H-pyrazol-1-yl}acetic acid;
3-{4-[3-(3-{[(2-methylpropoxy)carbonyl]amino}benzyl)-4-oxopyridazin-1(4H)-yl]-1H-pyrazol-1-yl}propanoic acid;
3-[3-(5-aminopyrimidin-2-yl)benzyl]-1-(1-methyl-1H-pyrazol-4-yl)pyridazin-4(1H)-one;
3-(1H-indazol-5-ylmethyl)-1-(1-methyl-1H-pyrazol-4-yl)pyridazin-4(1H)-one;
1-(1-methyl-1H-pyrazol-4-yl)-3-[3-(5-propyl-1H-1,2,4-triazol-3-yl)benzyl]pyridazin-4(1H)-one;
1-(1-methyl-1H-pyrazol-4-yl)-3-[3-(1H-1,2,4-triazol-3-yl)benzyl]pyridazin-4(1H)-one;
3-[3-(5-hydroxypyrimidin-2-yl)benzyl]-1-(1-methyl-1H-pyrazol-4-yl)pyridazin-4(1H)-one;
1-(3,4-difluorophenyl)-3-[3-(5-hydroxypyrimidin-2-yl)benzyl]pyridazin-4(1H)-one;
1-(3,5-difluorophenyl)-3-[3-(5-hydroxypyrimidin-2-yl)benzyl]pyridazin-4(1H)-one;
3-{3-[5-(hydroxymethyl)pyrimidin-2-yl]benzyl}-1-(1-methyl-1H-pyrazol-4-yl)pyridazin-4(1H)-one;
3-[3-(1-ethyl-1H-1,2,4-triazol-3-yl)benzyl]-1-[1-(2-hydroxyethyl)-1H-pyrazol-4-yl]pyridazin-4(1H)-one;
1-[1-(2-hydroxyethyl)-1H-pyrazol-4-yl]-3-[3-(1-propyl-1H-1,2,4-triazol-3-yl)benzyl]pyridazin-4(1H)-one;
3-[3-(5-ethoxypyrimidin-2-yl)benzyl]-1-[1-(2-hydroxyethyl)-1H-pyrazol-4-yl]pyridazin-4(1H)-one;
3-[3-(5-hydroxypyrimidin-2-yl)benzyl]-1-(1H-pyrazol-4-yl)pyridazin-4(1H)-one;
3-[3-(5-ethoxypyrimidin-2-yl)benzyl]-1-(1H-pyrazol-4-yl)pyridazin-4(1H)-one;
2-methoxyethyl(3-{[4-oxo-1-(1H-pyrazol-4-yl)-1,4-dihydropyridazin-3-yl]methyl}phenyl)carbamate;
isobutyl (3-{[4-oxo-1-(1H-pyrazol-4-yl)-1,4-dihydropyridazin-3-yl]methyl}phenyl)carbamate;
rac-1-(1-methyl-1H-pyrazol-4-yl)-3-{3-[5-(tetrahydrofuran-2-yl)pyrimidin-2-yl]benzyl}pyridazin-4(1H)-one;
rac-1-(1-methyl-1H-pyrazol-4-yl)-3-{3-[5-(tetrahydrofuran-3-yl)pyrimidin-2-yl]benzyl}pyridazin-4(1H)-one;
1-(1-methyl-1H-pyrazol-4-yl)-3-{3-[5-(piperidin-4-yl)pyrimidin-2-yl]benzyl}pyridazin-4(1H)-one;
3-{(1S or R)-1-[3-(5-ethoxypyrimidin-2-yl)phenyl]ethyl}-1-(1-methyl-1H-pyrazol-4-yl)pyridazin-4(1H)-one;
3-{(1R or S)-1-[3-(5-ethoxypyrimidin-2-yl)phenyl]ethyl}-1-(1-methyl-1H-pyrazol-4-yl)pyridazin-4(1H)-one;
3-{(1S or R)-1-[3-(5-methoxypyrimidin-2-yl)phenyl]ethyl}-1-(1-methyl-1H-pyrazol-4-yl)pyridazin-4(1H)-one;
3-{(1R or S)-1-[3-(5-methoxypyrimidin-2-yl)phenyl]ethyl}-1-(1-methyl-1H-pyrazol-4-yl)pyridazin-4(1H)-one;
3-{(1S or R)-1-[3-(5-ethoxypyrimidin-2-yl)phenyl]propyl}-1-(1-methyl-1H-pyrazol-4-yl)pyridazin-4(1H)-one;
3-{(1R or S)-1-[3-(5-ethoxypyrimidin-2-yl)phenyl]propyl}-1-(1-methyl-1H-pyrazol-4-yl)pyridazin-4(1H)-one;
3-{(1S or R)-1-[3-(5-ethoxypyrimidin-2-yl)phenyl]butyl}-1-(1-methyl-1H-pyrazol-4-yl)pyridazin-4(1H)-one;
3-{(1R or S)-1-[3-(5-ethoxypyrimidin-2-yl)phenyl]butyl}-1-(1-methyl-1H-pyrazol-4-yl)pyridazin-4(1H)-one;
3-[(1S or R)-1-{3-[5-(difluoromethoxy)pyrimidin-2-yl]phenyl}ethyl]-1-(1-methyl-1H-pyrazol-4-yl)pyridazin-4(1H)-one;
3-[(1R or S)-1-{3-[5-(difluoromethoxy)pyrimidin-2-yl]phenyl}ethyl]-1-(1-methyl-1H-pyrazol-4-yl)pyridazin-4(1H)-one;
1-(1-methyl-1H-pyrazol-4-yl)-3-[(1S or R)-1-{3-[5-(tetrahydro-2H-pyran-4-yloxy)pyrimidin-2-yl]phenyl}ethyl]pyridazin-4(1H)-one;
1-(1-methyl-1H-pyrazol-4-yl)-3-[(1R or S)-1-{3-[5-(tetrahydro-2H-pyran-4-yloxy)pyrimidin-2-yl]phenyl}ethyl]pyridazin-4(1H)-one;
1-(1-ethyl-1H-pyrazol-4-yl)-3-((1S or R)-1-{3-[5-(oxetan-3-yloxy)pyrimidin-2-yl]phenyl}ethyl)pyridazin-4(1H)-one;
1-(1-ethyl-1H-pyrazol-4-yl)-3-((1R or S)-1-{3-[5-(oxetan-3-yloxy)pyrimidin-2-yl]phenyl}ethyl)pyridazin-4(1H)-one;
1-(1-methyl-1H-pyrazol-4-yl)-3-((1S or R)-1-{3-[5-(oxetan-3-yloxy)pyrimidin-2-yl]phenyl}ethyl)pyridazin-4(1H)-one;
1-(1-methyl-1H-pyrazol-4-yl)-3-((1R or S)-1-{3-[5-(oxetan-3-yloxy)pyrimidin-2-yl]phenyl}ethyl)pyridazin-4(1H)-one;
1-(1-ethyl-1H-pyrazol-4-yl)-3-((1S or R)-1-{3-[5-(methoxymethyl)pyrimidin-2-yl]phenyl}ethyl)pyridazin-4(1H)-one;
1-(1-ethyl-1H-pyrazol-4-yl)-3-((1R or S)-1-{3-[5-(methoxymethyl)pyrimidin-2-yl]phenyl}ethyl)pyridazin-4(1H)-one;
3-((1S or R)-1-{3-[5-(methoxymethyl)pyrimidin-2-yl]phenyl}ethyl)-1-(1-methyl-1H-pyrazol-4-yl)pyridazin-4(1H)-one;
3-((1R or S)-1-{3-[5-(methoxymethyl)pyrimidin-2-yl]phenyl}ethyl)-1-(1-methyl-1H-pyrazol-4-yl)pyridazin-4(1H)-one;
rac-3-(1-{3-[5-(ethoxymethyl)pyrimidin-2-yl]phenyl}ethyl)-1-(1-methyl-1H-pyrazol-4-yl)pyridazin-4(1H)-one;
3-((1S or R)-1-{3-[5-(2-methoxyethoxy)pyrimidin-2-yl]phenyl}ethyl)-1-(1-methyl-1H-pyrazol-4-yl)pyridazin-4(1H)-one;
3-((1R or S)-1-{3-[5-(2-methoxyethoxy)pyrimidin-2-yl]phenyl}ethyl)-1-(1-methyl-1H-pyrazol-4-yl)pyridazin-4(1H)-one;
rac-3-[3-{1-[3-(5-ethoxypyrimidin-2-yl)phenyl]ethyl}-4-oxopyridazin-1(4H)-yl]benzonitrile;
rac-3-{1-[3-(5-methoxypyrimidin-2-yl)phenyl]propyl}-1-(1-methyl-1H-pyrazol-4-yl)pyridazin-4(1H)-one;
3-[(1S or R)-1-(3-ethoxyquinolin-6-yl)ethyl]-1-(1-ethyl-1H-pyrazol-4-yl)pyridazin-4(1H)-one;
3-[(1R or S)-1-(3-ethoxyquinolin-6-yl)ethyl]-1-(1-ethyl-1H-pyrazol-4-yl)pyridazin-4(1H)-one;
3-((1S or R)-1-{3-[5-(benzyloxy)pyrimidin-2-yl]phenyl}ethyl)-1-(1-ethyl-1H-pyrazol-4-yl)pyridazin-4(1H)-one;
3-((1R or S)-1-{3-[5-(benzyloxy)pyrimidin-2-yl]phenyl}ethyl)-1-(1-ethyl-1H-pyrazol-4-yl)pyridazin-4(1H)-one;
1-(3,4-difluorophenyl)-3-{(1S or R)-1-[3-(5-ethylpyrimidin-2-yl)phenyl]ethyl}pyridazin-4(1H)-one;
1-(3,4-difluorophenyl)-3-{(1R or S)-1-[3-(5-ethylpyrimidin-2-yl)phenyl]ethyl}pyridazin-4(1H)-one;
1-(1-methyl-1H-pyrazol-4-yl)-3-[(1S or R)-1-{3-[5-(1-methyl-1H-pyrazol-4-yl)pyrimidin-2-yl]phenyl}ethyl]pyridazin-4(1H)-one;
1-(1-methyl-1H-pyrazol-4-yl)-3-[(1R or S)-1-{3-[5-(1-methyl-1H-pyrazol-4-yl)pyrimidin-2-yl]phenyl}ethyl]pyridazin-4(1H)-one;
1-(1-ethyl-1H-pyrazol-4-yl)-3-[(1S or R)-1-{3-[5-(1-methyl-1H-pyrazol-4-yl)pyrimidin-2-yl]phenyl}ethyl]pyridazin-4(1H)-one;
1-(1-ethyl-1H-pyrazol-4-yl)-3-[(1R or S)-1-{3-[5-(1-methyl-1H-pyrazol-4-yl)pyrimidin-2-yl]phenyl}ethyl]pyridazin-4(1H)-one;
3-{(1S or R)-1-[3-(5-ethylpyrimidin-2-yl)phenyl]ethyl}-1-(1-methyl-1H-pyrazol-4-yl)pyridazin-4(1H)-one;
3-{(1R or S)-1-[3-(5-ethylpyrimidin-2-yl)phenyl]ethyl}-1-(1-methyl-1H-pyrazol-4-yl)pyridazin-4(1H)-one;
1-(3,4-difluorophenyl)-3-[(1S or R)-1-{3-[5-(1-methyl-1H-pyrazol-4-yl)pyrimidin-2-yl]phenyl}ethyl]pyridazin-4(1H)-one;
1-(3,4-difluorophenyl)-3-[(1R or S)-1-{3-[5-(1-methyl-1H-pyrazol-4-yl)pyrimidin-2-yl]phenyl}ethyl]pyridazin-4(1H)-one;
1-(1-methyl-1H-pyrazol-4-yl)-3-[(1R or S)-1-{3-[5-(4H-1,2,4-triazol-4-yl)pyrimidin-2-yl]phenyl}ethyl]pyridazin-4(1H)-one;
3-{(1S or R)-1-[3-(5-bromopyrimidin-2-yl)phenyl]ethyl}-1-(1-methyl-1H-pyrazol-4-yl)pyridazin-4(1H)-one;
3-{(1R or S)-1-[3-(5-bromopyrimidin-2-yl)phenyl]ethyl}-1-(1-methyl-1H-pyrazol-4-yl)pyridazin-4(1H)-one;
1-(1-methyl-1H-pyrazol-4-yl)-3-{(1S or R)-1-[3-(5-morpholin-4-ylpyrimidin-2-yl)phenyl]ethyl}pyridazin-4(1H)-one;
1-(1-methyl-1H-pyrazol-4-yl)-3-{(1R or S)-1-[3-(5-morpholin-4-ylpyrimidin-2-yl)phenyl]ethyl}pyridazin-4(1H)-one;
3-{(1S or R)-1-[3-(5-ethoxypyrimidin-2-yl)phenyl]ethyl}-1-(1-ethyl-1H-pyrazol-4-yl)pyridazin-4(1H)-one;
3-{(1R or S)-1-[3-(5-ethoxypyrimidin-2-yl)phenyl]ethyl}-1-(1-ethyl-1H-pyrazol-4-yl)pyridazin-4(1H)-one;
1-(1-ethyl-1H-pyrazol-4-yl)-3-((1S or R)-1-{3-[5-(2-methoxyethoxy)pyrimidin-2-yl]phenyl}ethyl)pyridazin-4(1H)-one;
1-(1-ethyl-1H-pyrazol-4-yl)-3-((1R or S)-1-{3-[5-(2-methoxyethoxy)pyrimidin-2-yl]phenyl}ethyl)pyridazin-4(1H)-one;
1-(3,4-difluorophenyl)-3-[(1S or R)-1-{3-[5-(2-hydroxy-2-methylpropoxy)pyrimidin-2-yl]phenyl}ethyl]pyridazin-4(1H)-one;
1-(3,4-difluorophenyl)-3-[(1R or S)-1-{3-[5-(2-hydroxy-2-methylpropoxy)pyrimidin-2-yl]phenyl}ethyl]pyridazin-4(1H)-one;
1-(3,4-difluorophenyl)-3-[(1S or R)-1-{3-[5-(2-methoxy-2-methylpropoxy)pyrimidin-2-yl]phenyl}ethyl]pyridazin-4(1H)-one;
1-(3,4-difluorophenyl)-3-[(1R or S)-1-{3-[5-(2-methoxy-2-methylpropoxy)pyrimidin-2-yl]phenyl}ethyl]pyridazin-4(1H)-one;
rac-3-(3-hydroxy-1-{3-[5-(2-methoxyethoxy)pyrimidin-2-yl]phenyl}propyl)-1-(1-methyl-1H-pyrazol-4-yl)pyridazin-4(1H)-one;
rac-3-{2-hydroxy-1-[3-(5-methoxypyrimidin-2-yl)phenyl]ethyl}-1-(1-methyl-1H-pyrazol-4-yl)pyridazin-4(1H)-one;
3-{(1S or R)-1-[3-(5-ethoxypyrimidin-2-yl)phenyl]ethyl}-1-[1-(2-hydroxyethyl)-1H-pyrazol-4-yl]pyridazin-4(1H)-one;
3-{(1R or S)-1-[3-(5-ethoxypyrimidin-2-yl)phenyl]ethyl}-1-[1-(2-hydroxyethyl)-1H-pyrazol-4-yl]pyridazin-4(1H)-one;
1-(1-ethyl-1H-pyrazol-4-yl)-3-{(1S or R)-1-[3-(5-hydroxypyrimidin-2-yl)phenyl]ethyl}pyridazin-4(1H)-one;
1-(1-ethyl-1H-pyrazol-4-yl)-3-{(1R or S)-1-[3-(5-hydroxypyrimidin-2-yl)phenyl]ethyl}pyridazin-4(1H)-one;
3-(3-{5-[2,2-difluoro-3-(morpholin-4-yl)propoxy]pyrimidin-2-yl}benzyl)-1-(1-methyl-1H-pyrazol-4-yl)pyridazin-4(1H)-one;
rac-1-(1-methyl-1H-pyrazol-4-yl)-3-{3-[5-(tetrahydrofuran-2-ylmethoxy)pyrimidin-2-yl]benzyl}pyridazin-4(1H)-one;
3-{3-[5-(2-methylpropoxy)pyrimidin-2-yl]benzyl}-1-(1-methyl-1H-pyrazol-4-yl)pyridazin-4(1H)-one;
1-(1-methyl-1H-pyrazol-4-yl)-3-{3-[5-(tetrahydro-2H-pyran-4-ylmethoxy)pyrimidin-2-yl]benzyl}pyridazin-4(1H)-one;
1-(1-methyl-1H-pyrazol-4-yl)-3-{3-[5-(2-morpholin-4-ylethoxy)pyrimidin-2-yl]benzyl}pyridazin-4(1H)-one;
3-{3-[5-(2-hydroxyethoxy)pyrimidin-2-yl]benzyl}-1-(1-methyl-1H-pyrazol-4-yl)pyridazin-4(1H)-one;
1-(1-methyl-1H-pyrazol-4-yl)-3-(3-{5-[2-(1H-pyrazol-1-yl)ethoxy]pyrimidin-2-yl}benzyl)pyridazin-4(1H)-one;
rac-1-(1-methyl-1H-pyrazol-4-yl)-3-{3-[5-(tetrahydrofuran-3-ylmethoxy)pyrimidin-2-yl]benzyl}pyridazin-4(1H)-one;
3-{3-[5-(2-methoxy-2-methylpropoxy)pyrimidin-2-yl]benzyl}-1-(1-methyl-1H-pyrazol-4-yl)pyridazin-4(1H)-one;
3-[3-(5-{[3-(hydroxymethyl)oxetan-3-yl]methoxy}pyrimidin-2-yl)benzyl]-1-(1-methyl-1H-pyrazol-4-yl)pyridazin-4(1H)-one;
rac-3-(3-{5-[(2,2-dimethyltetrahydro-2H-pyran-4-yl)methoxy]pyrimidin-2-yl}benzyl)-1-(1-methyl-1H-pyrazol-4-yl)pyridazin-4(1H)-one;
3-{3-[5-(1-methylethoxy)pyrimidin-2-yl]benzyl}-1-(1-methyl-1H-pyrazol-4-yl)pyridazin-4(1H)-one;
1-(1-methyl-1H-pyrazol-4-yl)-3-(3-{5-[2-(1H-1,2,4-triazol-1-yl)ethoxy]pyrimidin-2-yl}benzyl)pyridazin-4(1H)-one;
3-(3-{5-[(3-fluorooxetan-3-yl)methoxy]pyrimidin-2-yl}benzyl)-1-(1-methyl-1H-pyrazol-4-yl)pyridazin-4(1H)-one;
3-{3-[5-(2-isoxazol-4-ylethoxy)pyrimidin-2-yl]benzyl}-1-(1-methyl-1H-pyrazol-4-yl)pyridazin-4(1H)-one;
3-{3-[5-(2,2-difluoroethoxy)pyrimidin-2-yl]benzyl}-1-(1-methyl-1H-pyrazol-4-yl)pyridazin-4(1H)-one;
3-[3-(5-ethoxypyrimidin-2-yl)benzyl]-1-(1-methyl-1H-pyrazol-4-yl)pyridazin-4(1H)-one-d5;
rac-1-(1-methyl-1H-pyrazol-4-yl)-3-{3-[5-(tetrahydrofuran-3-yloxy)pyrimidin-2-yl]benzyl}pyridazin-4(1H)-one;
1-(1-methyl-1H-pyrazol-4-yl)-3-{3-[5-(tetrahydro-2H-pyran-4-yloxy)pyrimidin-2-yl]benzyl}pyridazin-4(1H)-one;
3-{3-[5-(cyclopropylmethoxy)pyrimidin-2-yl]benzyl}-1-(1-methyl-1H-pyrazol-4-yl)pyridazin-4(1H)-one;
N,N-dimethyl-2-{[2-(3-{[1-(1-methyl-1H-pyrazol-4-yl)-4-oxo-1,4-dihydropyridazin-3-yl]methyl}phenyl)pyrimidin-5-yl]oxy}acetamide;
1-(1-methyl-1H-pyrazol-4-yl)-3-{3-[5-(2-morpholin-4-yl-2-oxoethoxy)pyrimidin-2-yl]benzyl}pyridazin-4(1H)-one;
3-(3-{5-[(5-methyl-1,2,4-oxadiazol-3-yl)methoxy]pyrimidin-2-yl}benzyl)-1-(1-methyl-1H-pyrazol-4-yl)pyridazin-4(1H)-one;
3-(3-{5-[(5-cyclopropyl-1,2,4-oxadiazol-3-yl)methoxy]pyrimidin-2-yl}benzyl)-1-(1-methyl-1H-pyrazol-4-yl)pyridazin-4(1H)-one;
3-[3-(5-{[5-(1-methylethyl)-1,2,4-oxadiazol-3-yl]methoxy}pyrimidin-2-yl)benzyl]-1-(1-methyl-1H-pyrazol-4-yl)pyridazin-4(1H)-one;
3-{3-[5-(isothiazol-3-ylmethoxy)pyrimidin-2-yl]benzyl}-1-(1-methyl-1H-pyrazol-4-yl)pyridazin-4(1H)-one;
3-(3-{5-[(5-methylisoxazol-3-yl)methoxy]pyrimidin-2-yl}benzyl)-1-(1-methyl-1H-pyrazol-4-yl)pyridazin-4(1H)-one;
3-(3-{5-[(3-methylisoxazol-5-yl)methoxy]pyrimidin-2-yl}benzyl)-1-(1-methyl-1H-pyrazol-4-yl)pyridazin-4(1H)-one;
tert-butyl [2-(3-{[1-(1-methyl-1H-pyrazol-4-yl)-4-oxo-1,4-dihydropyridazin-3-yl]methyl}phenyl)pyrimidin-5-yl]oxyacetate;
tert-butyl 4-({[2-(3-{[1-(1-ethyl-1H-pyrazol-4-yl)-4-oxo-1,4-dihydropyridazin-3-yl]methyl}phenyl)pyrimidin-5-yl]oxy}methyl)piperidine-1-carboxylate;
tert-butyl 3-{[2-(3-{[1-(1-ethyl-1H-pyrazol-4-yl)-4-oxo-1,4-dihydropyridazin-3-yl]methyl}phenyl)pyrimidin-5-yl]oxy}azetidine-1-carboxylate;
tert-butyl 4-({[2-(3-{(1R or S)-1-[1-(1-ethyl-1H-pyrazol-4-yl)-4-oxo-1,4-dihydropyridazin-3-yl]ethyl}phenyl)pyrimidin-5-yl]oxy}methyl)-4-fluoropiperidine-1-carboxylate;
tert-butyl 4-({[2-(3-{(1R or S)-1-[1-(1-ethyl-1H-pyrazol-4-yl)-4-oxo-1,4-dihydropyridazin-3-yl]ethyl}phenyl)pyrimidin-5-yl]oxy}methyl)piperidine-1-carboxylate;
tert-butyl 3-{[2-(3-{(1R or S)-1-[1-(1-ethyl-1H-pyrazol-4-yl)-4-oxo-1,4-dihydropyridazin-3-yl]ethyl}phenyl)pyrimidin-5-yl]oxy}azetidine-1-carboxylate;
1-(1-ethyl-1H-pyrazol-4-yl)-3-[(1R or S)-1-{3-[5-(1-methylethoxy)pyrimidin-2-yl]phenyl}ethyl]pyridazin-4(1H)-one;
3-[(1R or S)-1-{3-[5-(2,2-difluoroethoxy)pyrimidin-2-yl]phenyl}ethyl]-1-(1-ethyl-1H-pyrazol-4-yl)pyridazin-4(1H)-one;
1-(1-ethyl-1H-pyrazol-4-yl)-3-[(1R or S)-1-{3-[5-(2-hydroxyethoxy)pyrimidin-2-yl]phenyl}ethyl]pyridazin-4(1H)-one;
1-(1-ethyl-1H-pyrazol-4-yl)-3-(1-{3-[5-(oxetan-2-ylmethoxy)pyrimidin-2-yl]phenyl}ethyl)pyridazin-4(1H)-one;
1-(1-ethyl-1H-pyrazol-4-yl)-3-(1-{3-[5-(tetrahydrofuran-3-yloxy)pyrimidin-2-yl]phenyl}ethyl)pyridazin-4(1H)-one;
1-(1-ethyl-1H-pyrazol-4-yl)-3-(1-{3-[5-(tetrahydrofuran-2-ylmethoxy)pyrimidin-2-yl]phenyl}ethyl)pyridazin-4(1H)-one;
3-(1-{3-[5-(1,4-dioxan-2-ylmethoxy)pyrimidin-2-yl]phenyl}ethyl)-1-(1-ethyl-1H-pyrazol-4-yl)pyridazin-4(1H)-one;
1-(1-ethyl-1H-pyrazol-4-yl)-3-[(1R or S)-1-{3-[5-(2-morpholin-4-ylethoxy)pyrimidin-2-yl]phenyl}ethyl]pyridazin-4(1H)-one;
1-(1-ethyl-1H-pyrazol-4-yl)-3-[(1R or S)-1-(3-{5-[(3-methylisoxazol-5-yl)methoxy]pyrimidin-2-yl}phenyl)ethyl]pyridazin-4(1H)-one;
1-(1-ethyl-1H-pyrazol-4-yl)-3-[(1R or S)-1-(3-{5-[(5-methyl-1,2,4-oxadiazol-3-yl)methoxy]pyrimidin-2-yl}phenyl)ethyl]pyridazin-4(1H)-one;
1-(1-ethyl-1H-pyrazol-4-yl)-3-[(1R or S)-1-(3-{5-[2-(1H-pyrazol-1-yl)ethoxy]pyrimidin-2-yl}phenyl)ethyl]pyridazin-4(1H)-one;
1-(1-ethyl-1H-pyrazol-4-yl)-3-[(1R or S)-1-(3-{5-[(3-methyloxetan-3-yl)methoxy]pyrimidin-2-yl}phenyl)ethyl]pyridazin-4(1H)-one;
1-(1-ethyl-1H-pyrazol-4-yl)-3-[3-(5-isopropoxypyrimidin-2-yl)benzyl]pyridazin-4(1H)-one;
3-{3-[5-(2,2-difluoroethoxy)pyrimidin-2-yl]benzyl}-1-(1-ethyl-1H-pyrazol-4-yl)pyridazin-4(1H)-one;
1-(1-ethyl-1H-pyrazol-4-yl)-3-{3-[5-(2-hydroxyethoxy)pyrimidin-2-yl]benzyl}pyridazin-4(1H)-one;
rac-1-(1-ethyl-1H-pyrazol-4-yl)-3-{3-[5-(oxetan-2-ylmethoxy)pyrimidin-2-yl]benzyl}pyridazin-4(1H)-one;
rac-1-(1-ethyl-1H-pyrazol-4-yl)-3-{3-[5-(tetrahydrofuran-3-yloxy)pyrimidin-2-yl]benzyl}pyridazin-4(1H)-one;
rac-1-(1-ethyl-1H-pyrazol-4-yl)-3-{3-[5-(tetrahydrofuran-2-ylmethoxy)pyrimidin-2-yl]benzyl}pyridazin-4(1H)-one;
rac-3-{3-[5-(1,4-dioxan-2-ylmethoxy)pyrimidin-2-yl]benzyl}-1-(1-ethyl-1H-pyrazol-4-yl)pyridazin-4(1H)-one;
1-(1-ethyl-1H-pyrazol-4-yl)-3-{3-[5-(2-morpholin-4-ylethoxy)pyrimidin-2-yl]benzyl}pyridazin-4(1H)-one;
1-(1-ethyl-1H-pyrazol-4-yl)-3-(3-{5-[(3-methylisoxazol-5-yl)methoxy]pyrimidin-2-yl}benzyl)pyridazin-4(1H)-one;
1-(1-ethyl-1H-pyrazol-4-yl)-3-(3-{5-[(5-methyl-1,2,4-oxadiazol-3-yl)methoxy]pyrimidin-2-yl}benzyl)pyridazin-4(1H)-one;
1-(1-ethyl-1H-pyrazol-4-yl)-3-(3-{5-[2-(1H-pyrazol-1-yl)ethoxy]pyrimidin-2-yl}benzyl)pyridazin-4(1H)-one;
1-(1-ethyl-1H-pyrazol-4-yl)-3-(3-{5-[(3-methyloxetan-3-yl)methoxy]pyrimidin-2-yl}benzyl)pyridazin-4(1H)-one;
1-(3,4-difluorophenyl)-3-{3-[5-(2-methoxyethoxy)pyrimidin-2-yl]benzyl}pyridazin-4(1H)-one;
3-{3-[5-(2-methoxyethoxy)pyrimidin-2-yl]benzyl}-1-(3,4,5-trifluorophenyl)pyridazin-4(1H)-one;
3-[3-{3-[5-(2-methoxyethoxy)pyrimidin-2-yl]benzyl}-4-oxopyridazin-1(4H)-yl]benzonitrile;
1-(3,4-difluorophenyl)-3-{3-[5-(3-methoxypropoxy)pyrimidin-2-yl]benzyl}pyridazin-4(1H)-one;
1-(3,4-difluorophenyl)-3-{3-[5-(2-ethoxyethoxy)pyrimidin-2-yl]benzyl}pyridazin-4(1H)-one;
3-[3-{3-[5-(3-methoxypropoxy)pyrimidin-2-yl]benzyl}-4-oxopyridazin-1(4H)-yl]benzonitrile;
3-[3-{3-[5-(2-ethoxyethoxy)pyrimidin-2-yl]benzyl}-4-oxopyridazin-1(4H)-yl]benzonitrile;
3-{3-[5-(3-methoxypropoxy)pyrimidin-2-yl]benzyl}-1-(3,4,5-trifluorophenyl)pyridazin-4(1H)-one;
3-{3-[5-(2-ethoxyethoxy)pyrimidin-2-yl]benzyl}-1-(3,4,5-trifluorophenyl)pyridazin-4(1H)-one;
1-(3,4-difluorophenyl)-3-{3-[5-(oxetan-3-yloxy)pyrimidin-2-yl]benzyl}pyridazin-4(1H)-one;
rac-1-(3,4-difluorophenyl)-3-{3-[5-(tetrahydrofuran-3-yloxy)pyrimidin-2-yl]benzyl}pyridazin-4(1H)-one;
rac-1-(3,4-difluorophenyl)-3-{3-[5-(tetrahydrofuran-3-ylmethoxy)pyrimidin-2-yl]benzyl}pyridazin-4(1H)-one;
1-(3,4-difluorophenyl)-3-(3-{5-[(3-methyloxetan-3-yl)methoxy]pyrimidin-2-yl}benzyl)pyridazin-4(1H)-one;
1-(3,5-difluorophenyl)-3-{3-[5-(oxetan-3-yloxy)pyrimidin-2-yl]benzyl}pyridazin-4(1H)-one;
1-(3,5-difluorophenyl)-3-{3-[5-(tetrahydro-2H-pyran-4-ylmethoxy)pyrimidin-2-yl]benzyl}pyridazin-4(1H)-one;
1-(1-ethyl-1H-pyrazol-4-yl)-3-{(1S or R)-1-[3-(5-methoxypyrimidin-2-yl)phenyl]ethyl}pyridazin-4(1H)-one;
1-(1-ethyl-1H-pyrazol-4-yl)-3-{(1R or S)-1-[3-(5-methoxypyrimidin-2-yl)phenyl]ethyl}pyridazin-4(1H)-one;
rac-1-(1-methyl-1H-pyrazol-4-yl)-3-(1-{3-[5-(piperidin-4-yloxy)pyrimidin-2-yl]phenyl}ethyl)pyridazin-4(1H)-one;
1-(1-ethyl-1H-pyrazol-4-yl)-3-(3-{5-[(4-fluoropiperidin-4-yl)methoxy]pyrimidin-2-yl}benzyl)pyridazin-4(1H)-one;
1-(1-ethyl-1H-pyrazol-4-yl)-3-{3-[5-(piperidin-4-ylmethoxy)pyrimidin-2-yl]benzyl}pyridazin-4(1H)-one;
3-{3-[5-(azetidin-3-yloxy)pyrimidin-2-yl]benzyl}-1-(1-ethyl-1H-pyrazol-4-yl)pyridazin-4(1H)-one;
1-(1-ethyl-1H-pyrazol-4-yl)-3-[(1R or S)-1-(3-{5-[(4-fluoropiperidin-4-yl)methoxy]pyrimidin-2-yl}phenyl)ethyl]pyridazin-4(1H)-one;
1-(1-ethyl-1H-pyrazol-4-yl)-3-((1R or S)-1-{3-[5-(piperidin-4-ylmethoxy)pyrimidin-2-yl]phenyl}ethyl)pyridazin-4(1H)-one;
3-((1R or S)-1-{3-[5-(azetidin-3-yloxy)pyrimidin-2-yl]phenyl}ethyl)-1-(1-ethyl-1H-pyrazol-4-yl)pyridazin-4(1H)-one;
3-[1-(3-{5-[(trans-3-fluoropiperidin-4-yl)oxy]pyrimidin-2-yl}phenyl)ethyl]-1-(1-methyl-1H-pyrazol-4-yl)pyridazin-4(1H)-one;
3-[1-(3-{5-[(cis-3-fluoropiperidin-4-yl)oxy]pyrimidin-2-yl}phenyl)ethyl]-1-(1-methyl-1H-pyrazol-4-yl)pyridazin-4(1H)-one;
1-(3,4-difluorophenyl)-3-(3-{5-[(2R or S)-1,4-dioxan-2-ylmethoxy]pyrimidin-2-yl}benzyl)pyridazin-4(1H)-one;
1-(3,4-difluorophenyl)-3-(3-{5-[(2S or R)-1,4-dioxan-2-ylmethoxy]pyrimidin-2-yl}benzyl)pyridazin-4(1H)-one;
3-(3-{5-[(2R or S)-1,4-dioxan-2-ylmethoxy]pyrimidin-2-yl}benzyl)-1-(1-methyl-1H-pyrazol-4-yl)pyridazin-4(1H)-one;
3-(3-{5-[(2S or R)-1,4-dioxan-2-ylmethoxy]pyrimidin-2-yl}benzyl)-1-(1-methyl-1H-pyrazol-4-yl)pyridazin-4(1H)-one;
1-(3,4-difluorophenyl)-3-(3-{5-[(3R or S)-tetrahydrofuran-3-yloxy]pyrimidin-2-yl}benzyl)pyridazin-4(1H)-one;
1-(3,4-difluorophenyl)-3-(3-{5-[(3S or R)-tetrahydrofuran-3-yloxy]pyrimidin-2-yl}benzyl)pyridazin-4(1H)-one;
1-(3,4-difluorophenyl)-3-(3-{5-[(3R or S)-tetrahydrofuran-3-ylmethoxy]pyrimidin-2-yl}benzyl)pyridazin-4(1H)-one;
1-(3,4-difluorophenyl)-3-(3-{5-[(3S or R)-tetrahydrofuran-3-ylmethoxy]pyrimidin-2-yl}benzyl)pyridazin-4(1H)-one;
1-(1-methyl-1H-pyrazol-4-yl)-3-{3-[5-(pyridin-4-ylmethoxy)pyrimidin-2-yl]benzyl}pyridazin-4(1H)-one;
1-(1-methyl-1H-pyrazol-4-yl)-3-{3-[5-(pyridin-2-ylmethoxy)pyrimidin-2-yl]benzyl}pyridazin-4(1H)-one;
1-(1-methyl-1H-pyrazol-4-yl)-3-{3-[5-(pyridin-3-ylmethoxy)pyrimidin-2-yl]benzyl}pyridazin-4(1H)-one;
1-(1-methyl-1H-pyrazol-4-yl)-3-(3-{5-[(1-methyl-1H-1,2,4-triazol-3-yl)methoxy]pyrimidin-2-yl}benzyl)pyridazin-4(1H)-one;
[2-(3-{[1-(1-methyl-1H-pyrazol-4-yl)-4-oxo-1,4-dihydropyridazin-3-yl]methyl}phenyl)pyrimidin-5-yl]oxyacetic acid;
3-{3-[5-(2-hydroxy-2-methylpropoxy)pyrimidin-2-yl]benzyl}-1-(1-methyl-1H-pyrazol-4-yl)pyridazin-4(1H)-one;
1-(1-ethyl-1H-pyrazol-4-yl)-3-{3-[5-(2-hydroxy-2-methylpropoxy)pyrimidin-2-yl]benzyl}pyridazin-4(1H)-one;
1-(3,4-difluorophenyl)-3-{3-[5-(2-hydroxy-2-methylpropoxy)pyrimidin-2-yl]benzyl}pyridazin-4(1H)-one;
3-[3-{3-[5-(2-hydroxy-2-methylpropoxy)pyrimidin-2-yl]benzyl}-4-oxopyridazin-1(4H)-yl]benzonitrile;
3-{3-[5-(2-hydroxy-2-methylpropoxy)pyrimidin-2-yl]benzyl}-1-(3,4,5-trifluorophenyl)pyridazin-4(1H)-one;
1-(3,5-difluorophenyl)-3-{3-[5-(2-hydroxy-2-methylpropoxy)pyrimidin-2-yl]benzyl}pyridazin-4(1H)-one;
rac-1-(1-methyl-1H-pyrazol-4-yl)-3-{3-[5-(3,3,3-trifluoro-2-hydroxypropoxy)pyrimidin-2-yl]benzyl}pyridazin-4(1H)-one;
3-(3-{5-[(4-hydroxytetrahydro-2H-pyran-4-yl)methoxy]pyrimidin-2-yl}benzyl)-1-(1-methyl-1H-pyrazol-4-yl)pyridazin-4(1H)-one;
rac-3-{3-[5-(2-hydroxy-1,2-dimethylpropoxy)pyrimidin-2-yl]benzyl}-1-(1-methyl-1H-pyrazol-4-yl)pyridazin-4(1H)-one;
rac-3-(3-{5-[2-hydroxy-2-(pyridin-4-yl)ethoxy]pyrimidin-2-yl}benzyl)-1-(1-methyl-1H-pyrazol-4-yl)pyridazin-4(1H)-one;
rac-3-{3-[5-(2-hydroxy-3-morpholin-4-ylpropoxy)pyrimidin-2-yl]benzyl}-1-(1-methyl-1H-pyrazol-4-yl)pyridazin-4(1H)-one;
rac-3-{3-[5-(3-fluoro-2-hydroxypropoxy)pyrimidin-2-yl]benzyl}-1-(1-methyl-1H-pyrazol-4-yl)pyridazin-4(1H)-one;
rac-3-{3-[5-(3-ethoxy-2-hydroxypropoxy)pyrimidin-2-yl]benzyl}-1-(1-methyl-1H-pyrazol-4-yl)pyridazin-4(1H)-one;
3-chloro-5-[3-{3-[5-(2-hydroxy-2-methylpropoxy)pyrimidin-2-yl]benzyl}-4-oxopyridazin-1(4H)-yl]benzonitrile;
4-[3-{3-[5-(2-hydroxy-2-methylpropoxy)pyrimidin-2-yl]benzyl}-4-oxopyridazin-1(4H)-yl]benzonitrile;
1-(3,4-difluorophenyl)-3-(3-{5-[(4-hydroxytetrahydro-2H-pyran-4-yl)methoxy]pyrimidin-2-yl}benzyl)pyridazin-4(1H)-one;
3-[3-(5-{[(1R,2R)-2-hydroxy-1-methylpropyl]oxy}pyrimidin-2-yl)benzyl]-1-(1-methyl-1H-pyrazol-4-yl)pyridazin-4(1H)-one;
3-[3-(5-{[(1S,2S)-2-hydroxy-1-methylpropyl]oxy}pyrimidin-2-yl)benzyl]-1-(1-methyl-1H-pyrazol-4-yl)pyridazin-4(1H)-one;
3-[3-(5-{[(1R,2S)-2-hydroxy-1-methylpropyl]oxy}pyrimidin-2-yl)benzyl]-1-(1-methyl-1H-pyrazol-4-yl)pyridazin-4(1H)-one;
3-[3-(5-{[(1S,2R)-2-hydroxy-1-methylpropyl]oxy}pyrimidin-2-yl)benzyl]-1-(1-methyl-1H-pyrazol-4-yl)pyridazin-4(1H)-one;
3-[3-(5-{[(1R,2R)-2-hydroxycyclopentyl]oxy}pyrimidin-2-yl)benzyl]-1-(1-methyl-1H-pyrazol-4-yl)pyridazin-4(1H)-one;
3-[3-(5-{[(1S,2S)-2-hydroxycyclopentyl]oxy}pyrimidin-2-yl)benzyl]-1-(1-methyl-1H-pyrazol-4-yl)pyridazin-4(1H)-one;
3-((1S or R)-1-{3-[5-(2-hydroxy-2-methylpropoxy)pyrimidin-2-yl]phenyl}ethyl)-1-(1-methyl-1H-pyrazol-4-yl)pyridazin-4(1H)-one;
3-((1R or S)-1-{3-[5-(2-hydroxy-2-methylpropoxy)pyrimidin-2-yl]phenyl}ethyl)-1-(1-methyl-1H-pyrazol-4-yl)pyridazin-4(1H)-one;
3-{(1R)-1-[3-(5-{[(1R)-2-hydroxy-1,2-dimethylpropyl]oxy}pyrimidin-2-yl)phenyl]ethyl}-1-(1-methyl-1H-pyrazol-4-yl)pyridazin-4(1H)-one;
3-{(1R)-1-[3-(5-{[(1S)-2-hydroxy-1,2-dimethylpropyl]oxy}pyrimidin-2-yl)phenyl]ethyl}-1-(1-methyl-1H-pyrazol-4-yl)pyridazin-4(1H)-one;
3-{(1S)-1-[3-(5-{[(1R)-2-hydroxy-1,2-dimethylpropyl]oxy}pyrimidin-2-yl)phenyl]ethyl}-1-(1-methyl-1H-pyrazol-4-yl)pyridazin-4(1H)-one;
3-{(1S)-1-[3-(5-{[(1S)-2-hydroxy-1,2-dimethylpropyl]oxy}pyrimidin-2-yl)phenyl]ethyl}-1-(1-methyl-1H-pyrazol-4-yl)pyridazin-4(1H)-one;
3-{3-[5-(difluoromethoxy)pyrimidin-2-yl]benzyl}-1-(1-methyl-1H-pyrazol-4-yl)pyridazin-4(1H)-one;
3-[3-(5-{[3-(fluoromethyl)oxetan-3-yl]methoxy}pyrimidin-2-yl)benzyl]-1-(1-methyl-1H-pyrazol-4-yl)pyridazin-4(1H)-one;
3-(3-{5-[(4-fluorotetrahydro-2H-pyran-4-yl)methoxy]pyrimidin-2-yl}benzyl)-1-(1-methyl-1H-pyrazol-4-yl)pyridazin-4(1H)-one;
rac-3-(3-{5-[2-fluoro-2-(pyridin-4-yl)ethoxy]pyrimidin-2-yl}benzyl)-1-(1-methyl-1H-pyrazol-4-yl)pyridazin-4(1H)-one;
rac-3-(3-{5-[(cis-4-fluorotetrahydrofuran-3-yl)oxy]pyrimidin-2-yl}benzyl)-1-(1-methyl-1H-pyrazol-4-yl)pyridazin-4(1H)-one;
3-[3-(5-ethylpyrimidin-2-yl)benzyl]-1-(1-methyl-1H-pyrazol-4-yl)pyridazin-4(1H)-one;
3-[3-(5-butylpyrimidin-2-yl)benzyl]-1-(1-methyl-1H-pyrazol-4-yl)pyridazin-4(1H)-one;
3-[3-(5-cyclopropylpyrimidin-2-yl)benzyl]-1-(1-methyl-1H-pyrazol-4-yl)pyridazin-4(1H)-one;
3-{3-[5-(2-methylpropyl)pyrimidin-2-yl]benzyl}-1-(1-methyl-1H-pyrazol-4-yl)pyridazin-4(1H)-one;
3-{3-[5-(3-hydroxypropyl)pyrimidin-2-yl]benzyl}-1-(1-methyl-1H-pyrazol-4-yl)pyridazin-4(1H)-one;
3-[3-(5-benzylpyrimidin-2-yl)benzyl]-1-(1-methyl-1H-pyrazol-4-yl)pyridazin-4(1H)-one;
1-(1-methyl-1H-pyrazol-4-yl)-3-{3-[5-(2-phenylethyl)pyrimidin-2-yl]benzyl}pyridazin-4(1H)-one;
1-(1-methyl-1H-pyrazol-4-yl)-3-(3-{5-[2-(pyridin-2-yl)ethyl]pyrimidin-2-yl}benzyl)pyridazin-4(1H)-one;
1-(1-methyl-1H-pyrazol-4-yl)-3-{3-[5-(prop-1-en-2-yl)pyrimidin-2-yl]benzyl}pyridazin-4(1H)-one;
1-(1-methyl-1H-pyrazol-4-yl)-3-(3-{5-[(1E)-prop-1-en-1-yl]pyrimidin-2-yl}benzyl)pyridazin-4(1H)-one;
3-(3-{5-[(1E)-3-hydroxy-3-methylbut-1-en-1-yl]pyrimidin-2-yl}benzyl)-1-(1-methyl-1H-pyrazol-4-yl)pyridazin-4(1H)-one;
3-(3-{5-[(1E)-3-methoxyprop-1-en-1-yl]pyrimidin-2-yl}benzyl)-1-(1-methyl-1H-pyrazol-4-yl)pyridazin-4(1H)-one;
3-(3-{5-[(1E)-3-(dimethylamino)prop-1-en-1-yl]pyrimidin-2-yl}benzyl)-1-(1-methyl-1H-pyrazol-4-yl)pyridazin-4(1H)-one;
3-{3-[5-(furan-2-yl)pyrimidin-2-yl]benzyl}-1-(1-methyl-1H-pyrazol-4-yl)pyridazin-4(1H)-one;
3-{(1S or R)-1-[3-(5-cyclopropylpyrimidin-2-yl)phenyl]ethyl}-1-(1-methyl-1H-pyrazol-4-yl)pyridazin-4(1H)-one;
3-{(1R or S)-1-[3-(5-cyclopropylpyrimidin-2-yl)phenyl]ethyl}-1-(1-methyl-1H-pyrazol-4-yl)pyridazin-4(1H)-one;
1-(3,4-difluorophenyl)-3-[3-(5-ethylpyrimidin-2-yl)benzyl]pyridazin-4(1H)-one;
rac-3-{3-[5-(butan-2-yl)pyrimidin-2-yl]benzyl}-1-(1-methyl-1H-pyrazol-4-yl)pyridazin-4(1H)-one;
1-(1-methyl-1H-pyrazol-4-yl)-3-{3-[5-(pyridin-4-yl)pyrimidin-2-yl]benzyl}pyridazin-4(1H)-one;
1-(1-methyl-1H-pyrazol-4-yl)-3-{3-[5-(1H-pyrazol-4-yl)pyrimidin-2-yl]benzyl}pyridazin-4(1H)-one;
3-[3-(5,5′-bipyrimidin-2-yl)benzyl]-1-(1-methyl-1H-pyrazol-4-yl)pyridazin-4(1H)-one;
1-(1-methyl-1H-pyrazol-4-yl)-3-[3-(5-pyridin-3-ylpyrimidin-2-yl)benzyl]pyridazin-4(1H)-one;
5-[2-(3-{[1-(1-methyl-1H-pyrazol-4-yl)-4-oxo-1,4-dihydropyridazin-3-yl]methyl}phenyl)pyrimidin-5-yl]pyridine-2-carbonitrile;
3-{3-[5-(5-fluoropyridin-3-yl)pyrimidin-2-yl]benzyl}-1-(1-methyl-1H-pyrazol-4-yl)pyridazin-4(1H)-one;
3-{3-[5-(3-methoxypyridin-4-yl)pyrimidin-2-yl]benzyl}-1-(1-methyl-1H-pyrazol-4-yl)pyridazin-4(1H)-one;
1-(1-methyl-1H-pyrazol-4-yl)-3-{3-[5-(3-methylpyridin-4-yl)pyrimidin-2-yl]benzyl}pyridazin-4(1H)-one;
3-[3-(2′-amino-5,5′-bipyrimidin-2-yl)benzyl]-1-(1-methyl-1H-pyrazol-4-yl)pyridazin-4(1H)-one;
3-{3-[5-(5-fluoropyridin-2-yl)pyrimidin-2-yl]benzyl}-1-(1-methyl-1H-pyrazol-4-yl)pyridazin-4(1H)-one;
3-{3-[5-(6-aminopyridin-3-yl)pyrimidin-2-yl]benzyl}-1-(1-methyl-1H-pyrazol-4-yl)pyridazin-4(1H)-one;
1-(1-methyl-1H-pyrazol-4-yl)-3-{3-[5-(1H-pyrazol-3-yl)pyrimidin-2-yl]benzyl}pyridazin-4(1H)-one;
1-(1-methyl-1H-pyrazol-4-yl)-3-{3-[5-(1-methyl-1H-pyrazol-3-yl)pyrimidin-2-yl]benzyl}pyridazin-4(1H)-one;
1-(1-methyl-1H-pyrazol-4-yl)-3-{3-[5-(1,3-thiazol-4-yl)pyrimidin-2-yl]benzyl}pyridazin-4(1H)-one;
3-[3-(5-isoxazol-4-ylpyrimidin-2-yl)benzyl]-1-(1-methyl-1H-pyrazol-4-yl)pyridazin-4(1H)-one;
3-{3-[5-(3,5-dimethylisoxazol-4-yl)pyrimidin-2-yl]benzyl}-1-(1-methyl-1H-pyrazol-4-yl)pyridazin-4(1H)-one;
1-(1-methyl-1H-pyrazol-4-yl)-3-[3-(5-pyridazin-4-ylpyrimidin-2-yl)benzyl]pyridazin-4(1H)-one;
1-(1-methyl-1H-pyrazol-4-yl)-3-{3-[5-(morpholin-4-ylmethyl)pyrimidin-2-yl]benzyl}pyridazin-4(1H)-one;
3-(3-{5-[(methylamino)methyl]pyrimidin-2-yl}benzyl)-1-(1-methyl-1H-pyrazol-4-yl)pyridazin-4(1H)-one;
1-(1-methyl-1H-pyrazol-4-yl)-3-{3-[5-(thiomorpholin-4-ylmethyl)pyrimidin-2-yl]benzyl}pyridazin-4(1H)-one;
1-(1-methyl-1H-pyrazol-4-yl)-3-{3-[5-(pyrrolidin-1-ylmethyl)pyrimidin-2-yl]benzyl}pyridazin-4(1H)-one;
3-(3-{5-[(dimethylamino)methyl]pyrimidin-2-yl}benzyl)-1-(1-methyl-1H-pyrazol-4-yl)pyridazin-4(1H)-one;
rac-3-(3-{5-[(3-fluoropyrrolidin-1-yl)methyl]pyrimidin-2-yl}benzyl)-1-(1-methyl-1H-pyrazol-4-yl)pyridazin-4(1H)-one;
3-(3-{5-[(cyclohexylamino)methyl]pyrimidin-2-yl}benzyl)-1-(1-methyl-1H-pyrazol-4-yl)pyridazin-4(1H)-one;
1-(1-methyl-1H-pyrazol-4-yl)-3-{3-[5-(1-oxetan-3-yl-1H-pyrazol-4-yl)pyrimidin-2-yl]benzyl}pyridazin-4(1H)-one;
3-(3-{5-[(4-aminopiperidin-1-yl)methyl]pyrimidin-2-yl}benzyl)-1-(1-methyl-1H-pyrazol-4-yl)pyridazin-4(1H)-one;
1-(1-methyl-1H-pyrazol-4-yl)-3-{3-[5-(1-oxetan-3-yl-1H-pyrazol-4-yl)pyrimidin-2-yl]benzyl}pyridazin-4(1H)-one;
1-(1-methyl-1H-pyrazol-4-yl)-3-{3-[5-(propan-2-yl)pyrimidin-2-yl]benzyl}pyridazin-4(1H)-one;
3-{3-[5-(3-hydroxy-3-methylbutyl)pyrimidin-2-yl]benzyl}-1-(1-methyl-1H-pyrazol-4-yl)pyridazin-4(1H)-one;
3-(3-{5-[3-(dimethylamino)propyl]pyrimidin-2-yl}benzyl)-1-(1-methyl-1H-pyrazol-4-yl)pyridazin-4(1H)-one;
3-{3-[5-(3-methoxypropyl)pyrimidin-2-yl]benzyl}-1-(1-methyl-1H-pyrazol-4-yl)pyridazin-4(1H)-one;
rac-3-{3-[5-(3-hydroxypyrrolidin-1-yl)pyrimidin-2-yl]benzyl}-1-(1-methyl-1H-pyrazol-4-yl)pyridazin-4(1H)-one;
1-(1-methyl-1H-pyrazol-4-yl)-3-[3-(5-piperidin-1-ylpyrimidin-2-yl)benzyl]pyridazin-4(1H)-one;
3-{3-[5-(4-hydroxypiperidin-1-yl)pyrimidin-2-yl]benzyl}-1-(1-methyl-1H-pyrazol-4-yl)pyridazin-4(1H)-one;
1-(1-methyl-1H-pyrazol-4-yl)-3-{3-[5-(octahydroisoquinolin-2(1H)-yl)pyrimidin-2-yl]benzyl}pyridazin-4(1H)-one;
3-(3-{5-[4-(dimethylamino)piperidin-1-yl]pyrimidin-2-yl}benzyl)-1-(1-methyl-1H-pyrazol-4-yl)pyridazin-4(1H)-one;
1-[2-(3-{[1-(1-methyl-1H-pyrazol-4-yl)-4-oxo-1,4-dihydropyridazin-3-yl]methyl}phenyl)pyrimidin-5-yl]piperidine-4-carboxamide;
rac-1-[2-(3-{[1-(1-methyl-1H-pyrazol-4-yl)-4-oxo-1,4-dihydropyridazin-3-yl]methyl}phenyl)pyrimidin-5-yl]piperidine-3-carbonitrile;
3-{3-[5-(3,3-difluoropyrrolidin-1-yl)pyrimidin-2-yl]benzyl}-1-(1-methyl-1H-pyrazol-4-yl)pyridazin-4(1H)-one;
3-{3-[5-(1,1-dioxidothiomorpholin-4-yl)pyrimidin-2-yl]benzyl}-1-(1-methyl-1H-pyrazol-4-yl)pyridazin-4(1H)-one;
rac-3-(3-{5-[3-(methoxymethyl)piperidin-1-yl]pyrimidin-2-yl}benzyl)-1-(1-methyl-1H-pyrazol-4-yl)pyridazin-4(1H)-one;
rac-3-{3-[5-(3-methylmorpholin-4-yl)pyrimidin-2-yl]benzyl}-1-(1-methyl-1H-pyrazol-4-yl)pyridazin-4(1H)-one;
1-(1-methyl-1H-pyrazol-4-yl)-3-{3-[5-(propylamino)pyrimidin-2-yl]benzyl}pyridazin-4(1H)-one;
3-{3-[5-(ethylamino)pyrimidin-2-yl]benzyl}-1-(1-methyl-1H-pyrazol-4-yl)pyridazin-4(1H)-one;
3-(3-{5-[(2-methoxyethyl)amino]pyrimidin-2-yl}benzyl)-1-(1-methyl-1H-pyrazol-4-yl)pyridazin-4(1H)-one;
3-(3-{5-[(2-ethoxyethyl)amino]pyrimidin-2-yl}benzyl)-1-(1-methyl-1H-pyrazol-4-yl)pyridazin-4(1H)-one;
1-(1-methyl-1H-pyrazol-4-yl)-3-(3-{5-[(tetrahydrofuran-3-ylmethyl)amino]pyrimidin-2-yl}benzyl)pyridazin-4(1H)-one;
3-(3-{5-[methyl(propyl)amino]pyrimidin-2-yl}benzyl)-1-(1-methyl-1H-pyrazol-4-yl)pyridazin-4(1H)-one;
3-(3-{5-[(2-methoxyethyl)(methyl)amino]pyrimidin-2-yl}benzyl)-1-(1-methyl-1H-pyrazol-4-yl)pyridazin-4(1H)-one;
3-methoxy-N-[2-(3-{[1-(1-methyl-1H-pyrazol-4-yl)-4-oxo-1,4-dihydropyridazin-3-yl]methyl}phenyl)pyrimidin-5-yl]propanamide;
N-[2-(3-{[1-(1-methyl-1H-pyrazol-4-yl)-4-oxo-1,4-dihydropyridazin-3-yl]methyl}phenyl)pyrimidin-5-yl]-2-(tetrahydro-2H-pyran-4-yl)acetamide;
N-[2-(3-{[1-(1-methyl-1H-pyrazol-4-yl)-4-oxo-1,4-dihydropyridazin-3-yl]methyl}phenyl)pyrimidin-5-yl]propanamide;
2-methoxy-N-[2-(3-{[1-(1-methyl-1H-pyrazol-4-yl)-4-oxo-1,4-dihydropyridazin-3-yl]methyl}phenyl)pyrimidin-5-yl]acetamide;
rac-N-[2-(3-{[1-(1-methyl-1H-pyrazol-4-yl)-4-oxo-1,4-dihydropyridazin-3-yl]methyl}phenyl)pyrimidin-5-yl]tetrahydrofuran-2-carboxamide;
N-[2-(3-{[1-(1-methyl-1H-pyrazol-4-yl)-4-oxo-1,4-dihydropyridazin-3-yl]methyl}phenyl)pyrimidin-5-yl]-2-(2-oxopyrrolidin-1-yl)acetamide;
rac-N-[2-(3-{[1-(1-methyl-1H-pyrazol-4-yl)-4-oxo-1,4-dihydropyridazin-3-yl]methyl}phenyl)pyrimidin-5-yl]-2-(tetrahydrofuran-2-yl)acetamide;
3-[3-(5-bromopyrimidin-2-yl)benzyl]-1-(1-methyl-1H-pyrazol-4-yl)pyridazin-4(1H)-one;
3-{3-[5-(4-methylpiperazin-1-yl)pyrimidin-2-yl]benzyl}-1-(1-methyl-1H-pyrazol-4-yl)pyridazin-4(1H)-one;
rac-3-{3-[5-(3-fluoropiperidin-1-yl)pyrimidin-2-yl]benzyl}-1-(1-methyl-1H-pyrazol-4-yl)pyridazin-4(1H)-one;
rac-3-{3-[5-(3-methylpiperidin-1-yl)pyrimidin-2-yl]benzyl}-1-(1-methyl-1H-pyrazol-4-yl)pyridazin-4(1H)-one;
1-(1-methyl-1H-pyrazol-4-yl)-3-[3-(5-pyrrolidin-1-ylpyrimidin-2-yl)benzyl]pyridazin-4(1H)-one;
tert-butyl 4-[2-(3-{[1-(1-methyl-1H-pyrazol-4-yl)-4-oxo-1,4-dihydropyridazin-3-yl]methyl}phenyl)pyrimidin-5-yl]piperazine-1-carboxylate;
1-(1-methyl-1H-pyrazol-4-yl)-3-[3-(5-morpholin-4-ylpyrimidin-2-yl)benzyl]pyridazin-4(1H)-one;
1-(1-methyl-1H-pyrazol-4-yl)-3-{3-[5-(4H-1,2,4-triazol-4-yl)pyrimidin-2-yl]benzyl}pyridazin-4(1H)-one;
rac-2-(3-{[1-(1-methyl-1H-pyrazol-4-yl)-4-oxo-1,4-dihydropyridazin-3-yl]methyl}phenyl)-N-(tetrahydrofuran-3-ylmethyl)pyrimidine-5-carboxamide;
rac-N-(1,4-dioxan-2-ylmethyl)-2-(3-{[1-(1-methyl-1H-pyrazol-4-yl)-4-oxo-1,4-dihydropyridazin-3-yl]methyl}phenyl)pyrimidine-5-carboxamide;
2-(3-{[1-(1-methyl-1H-pyrazol-4-yl)-4-oxo-1,4-dihydropyridazin-3-yl]methyl}phenyl)-N-(2-morpholin-4-ylethyl)pyrimidine-5-carboxamide;
rac-2-(3-{[1-(1-methyl-1H-pyrazol-4-yl)-4-oxo-1,4-dihydropyridazin-3-yl]methyl}phenyl)-N-(tetrahydro-2H-pyran-3-ylmethyl)pyrimidine-5-carboxamide;
N-[3-(4-methylpiperazin-1-yl)propyl]-2-(3-{[1-(1-methyl-1H-pyrazol-4-yl)-4-oxo-1,4-dihydropyridazin-3-yl]methyl}phenyl)pyrimidine-5-carboxamide;
N-(2-methylpropyl)-2-(3-{[1-(1-methyl-1H-pyrazol-4-yl)-4-oxo-1,4-dihydropyridazin-3-yl]methyl}phenyl)pyrimidine-5-carboxamide;
2-(3-{[1-(1-methyl-1H-pyrazol-4-yl)-4-oxo-1,4-dihydropyridazin-3-yl]methyl}phenyl)-N-(2,2,2-trifluoroethyl)pyrimidine-5-carboxamide;
2-(3-{[1-(1-methyl-1H-pyrazol-4-yl)-4-oxo-1,4-dihydropyridazin-3-yl]methyl}phenyl)-N-(3-morpholin-4-ylpropyl)pyrimidine-5-carboxamide;
rac-2-(3-{[1-(1-methyl-1H-pyrazol-4-yl)-4-oxo-1,4-dihydropyridazin-3-yl]methyl}phenyl)-N-(tetrahydrofuran-2-ylmethyl)pyrimidine-5-carboxamide;
N-ethyl-2-(3-{[1-(1-methyl-1H-pyrazol-4-yl)-4-oxo-1,4-dihydropyridazin-3-yl]methyl}phenyl)pyrimidine-5-carboxamide;
N-methyl-2-(3-{[1-(1-methyl-1H-pyrazol-4-yl)-4-oxo-1,4-dihydropyridazin-3-yl]methyl}phenyl)pyrimidine-5-carboxamide;
1-ethyl-3-{3-[(4-oxo-1-phenyl-1,4-dihydropyridazin-3-yl)methyl]phenyl}urea;
1-methyl-3-(3-{[1-(1-methyl-1H-pyrazol-4-yl)-4-oxo-1,4-dihydropyridazin-3-yl]methyl}phenyl)urea;
1-ethyl-3-(3-{[1-(1-methyl-1H-pyrazol-4-yl)-4-oxo-1,4-dihydropyridazin-3-yl]methyl}phenyl)urea;
1-(3-{[1-(1-methyl-1H-pyrazol-4-yl)-4-oxo-1,4-dihydropyridazin-3-yl]methyl}phenyl)-3-propylurea;
1-benzyl-3-(3-{[1-(1-methyl-1H-pyrazol-4-yl)-4-oxo-1,4-dihydropyridazin-3-yl]methyl}phenyl)urea;
1-(2-methylpropyl)-3-(3-{[1-(1-methyl-1H-pyrazol-4-yl)-4-oxo-1,4-dihydropyridazin-3-yl]methyl}phenyl)urea;
1-cyclopropyl-3-(3-{[1-(1-methyl-1H-pyrazol-4-yl)-4-oxo-1,4-dihydropyridazin-3-yl]methyl}phenyl)urea;
1-(2-methoxyethyl)-3-(3-{[1-(1-methyl-1H-pyrazol-4-yl)-4-oxo-1,4-dihydropyridazin-3-yl]methyl}phenyl)urea;
1-butyl-3-(3-{[1-(1-methyl-1H-pyrazol-4-yl)-4-oxo-1,4-dihydropyridazin-3-yl]methyl}phenyl)urea;
1-(4-methoxybenzyl)-3-(3-{[1-(1-methyl-1H-pyrazol-4-yl)-4-oxo-1,4-dihydropyridazin-3-yl]methyl}phenyl)urea;
1-(3-{[1-(3,4-difluorophenyl)-4-oxo-1,4-dihydropyridazin-3-yl]methyl}phenyl)-3-(2-morpholin-4-ylethyl)urea;
methyl(3-{[1-(1-methyl-1H-pyrazol-4-yl)-4-oxo-1,4-dihydropyridazin-3-yl]methyl}phenyl)carbamate;
benzyl (3-{[1-(1-methyl-1H-pyrazol-4-yl)-4-oxo-1,4-dihydropyridazin-3-yl]methyl}phenyl)carbamate;
2-fluoroethyl(3-{[1-(1-methyl-1H-pyrazol-4-yl)-4-oxo-1,4-dihydropyridazin-3-yl]methyl}phenyl)carbamate;
butyl (3-{[1-(1-methyl-1H-pyrazol-4-yl)-4-oxo-1,4-dihydropyridazin-3-yl]methyl}phenyl)carbamate;
2,2-dimethylpropyl (3-{[1-(1-methyl-1H-pyrazol-4-yl)-4-oxo-1,4-dihydropyridazin-3-yl]methyl}phenyl)carbamate;
2-methoxyethyl(3-{[1-(3-cyanophenyl)-4-oxo-1,4-dihydropyridazin-3-yl]methyl}phenyl)carbamate;
2-methoxyethyl(3-{[4-oxo-1-(3,4,5-trifluorophenyl)-1,4-dihydropyridazin-3-yl]methyl}phenyl)carbamate;
ethyl(3-{[1-(3,4-difluorophenyl)-4-oxo-1,4-dihydropyridazin-3-yl]methyl}phenyl)carbamate;
2-methylpropyl (3-{[1-(3,4-difluorophenyl)-4-oxo-1,4-dihydropyridazin-3-yl]methyl}phenyl)carbamate;
2-methoxyethyl(3-{[1-(3,4-difluorophenyl)-4-oxo-1,4-dihydropyridazin-3-yl]methyl}phenyl)carbamate;
2-methoxyethyl(3-{[1-(3,5-difluorophenyl)-4-oxo-1,4-dihydropyridazin-3-yl]methyl}phenyl)carbamate;
2-methoxyethyl(3-{[1-(4-cyanophenyl)-4-oxo-1,4-dihydropyridazin-3-yl]methyl}phenyl)carbamate;
2-methoxyethyl(3-{[1-(3-chloro-5-fluorophenyl)-4-oxo-1,4-dihydropyridazin-3-yl]methyl}phenyl)carbamate;
2-(1H-imidazol-1-yl)ethyl(3-{[1-(1-methyl-1H-pyrazol-4-yl)-4-oxo-1,4-dihydropyridazin-3-yl]methyl}phenyl)carbamate;
3-(4-methylpiperazin-1-yl)propyl (3-{[4-oxo-1-(3,4,5-trifluorophenyl)-1,4-dihydropyridazin-3-yl]methyl}phenyl)carbamate;
1-(2-{[(3-{[4-oxo-1-(3,4,5-trifluorophenyl)-1,4-dihydropyridazin-3-yl]methyl}phenyl)carbamoyl]oxy}ethyl)piperidine-4-carboxylic acid;
rac-1,4-dioxan-2-ylmethyl(3-{[4-oxo-1-(3,4,5-trifluorophenyl)-1,4-dihydropyridazin-3-yl]methyl}phenyl)carbamate;
3-hydroxy-3-methylbutyl (3-{[4-oxo-1-(3,4,5-trifluorophenyl)-1,4-dihydropyridazin-3-yl]methyl}phenyl)carbamate;
2-(1,1-dioxidothiomorpholin-4-yl)ethyl(3-{[4-oxo-1-(3,4,5-trifluorophenyl)-1,4-dihydropyridazin-3-yl]methyl}phenyl)carbamate;
2-(4-methylpiperazin-1-yl)ethyl(3-{[4-oxo-1-(3,4,5-trifluorophenyl)-1,4-dihydropyridazin-3-yl]methyl}phenyl)carbamate
2-(1,1-dioxidothiomorpholin-4-yl)ethyl(3-{[1-(3,5-difluorophenyl)-4-oxo-1,4-dihydropyridazin-3-yl]methyl}phenyl)carbamate;
3-(4-methylpiperazin-1-yl)propyl (3-{[1-(3,5-difluorophenyl)-4-oxo-1,4-dihydropyridazin-3-yl]methyl}phenyl)carbamate;
2-(4-methylpiperazin-1-yl)ethyl(3-{[1-(3,5-difluorophenyl)-4-oxo-1,4-dihydropyridazin-3-yl]methyl}phenyl)carbamate;
rac-1,4-dioxan-2-ylmethyl(3-{[1-(3,5-difluorophenyl)-4-oxo-1,4-dihydropyridazin-3-yl]methyl}phenyl)carbamate;
3-morpholin-4-ylpropyl (3-{[1-(3,5-difluorophenyl)-4-oxo-1,4-dihydropyridazin-3-yl]methyl}phenyl)carbamate;
3-(1,1-dioxidothiomorpholin-4-yl)propyl (3-{[1-(3,5-difluorophenyl)-4-oxo-1,4-dihydropyridazin-3-yl]methyl}phenyl)carbamate;
2-morpholin-4-ylethyl(3-{[1-(3,5-difluorophenyl)-4-oxo-1,4-dihydropyridazin-3-yl]methyl}phenyl)carbamate;
2-(3,3-difluoropyrrolidin-1-yl)ethyl(3-{[1-(1-methyl-1H-pyrazol-4-yl)-4-oxo-1,4-dihydropyridazin-3-yl]methyl}phenyl)carbamate;
2,2-difluoro-3-morpholin-4-ylpropyl (3-{[1-(1-methyl-1H-pyrazol-4-yl)-4-oxo-1,4-dihydropyridazin-3-yl]methyl}phenyl)carbamate;
3-hydroxy-3-methylbutyl (3-{[1-(1-methyl-1H-pyrazol-4-yl)-4-oxo-1,4-dihydropyridazin-3-yl]methyl}phenyl)carbamate;
tetrahydro-2H-pyran-4-ylmethyl(3-{[1-(3,4-difluorophenyl)-4-oxo-1,4-dihydropyridazin-3-yl]methyl}phenyl)carbamate;
2-(1,1-dioxidothiomorpholin-4-yl)ethyl(3-{[1-(3,4-difluorophenyl)-4-oxo-1,4-dihydropyridazin-3-yl]methyl}phenyl)carbamate;
2-(3-oxopiperazin-1-yl)ethyl(3-{[1-(3,4-difluorophenyl)-4-oxo-1,4-dihydropyridazin-3-yl]methyl}phenyl)carbamate;
2-(4-methylpiperazin-1-yl)ethyl(3-{[1-(1-methyl-1H-pyrazol-4-yl)-4-oxo-1,4-dihydropyridazin-3-yl]methyl}phenyl)carbamate;
2-morpholin-4-ylethyl(3-{[1-(1-methyl-1H-pyrazol-4-yl)-4-oxo-1,4-dihydropyridazin-3-yl]methyl}phenyl)carbamate;
2-(1H-imidazol-1-yl)ethyl(3-{[1-(1-methyl-1H-pyrazol-4-yl)-4-oxo-1,4-dihydropyridazin-3-yl]methyl}phenyl)carbamate;
2-(2-oxopyrrolidin-1-yl)ethyl(3-{[1-(1-methyl-1H-pyrazol-4-yl)-4-oxo-1,4-dihydropyridazin-3-yl]methyl}phenyl)carbamate;
2-(1H-1,2,4-triazol-1-yl)ethyl(3-{[1-(1-methyl-1H-pyrazol-4-yl)-4-oxo-1,4-dihydropyridazin-3-yl]methyl}phenyl)carbamate;
2-(3-oxomorpholin-4-yl)ethyl(3-{[1-(1-methyl-1H-pyrazol-4-yl)-4-oxo-1,4-dihydropyridazin-3-yl]methyl}phenyl)carbamate;
3-(4-methylpiperazin-1-yl)propyl (3-{[1-(1-methyl-1H-pyrazol-4-yl)-4-oxo-1,4-dihydropyridazin-3-yl]methyl}phenyl)carbamate;
3-morpholin-4-ylpropyl (3-{[1-(1-methyl-1H-pyrazol-4-yl)-4-oxo-1,4-dihydropyridazin-3-yl]methyl}phenyl)carbamate;
cyclobutylmethyl(3-{[1-(1-methyl-1H-pyrazol-4-yl)-4-oxo-1,4-dihydropyridazin-3-yl]methyl}phenyl)carbamate;
cyclopentylmethyl(3-{[1-(1-methyl-1H-pyrazol-4-yl)-4-oxo-1,4-dihydropyridazin-3-yl]methyl}phenyl)carbamate;
cyclohexylmethyl(3-{[1-(1-methyl-1H-pyrazol-4-yl)-4-oxo-1,4-dihydropyridazin-3-yl]methyl}phenyl)carbamate;
tetrahydro-2H-pyran-4-ylmethyl(3-{[1-(1-methyl-1H-pyrazol-4-yl)-4-oxo-1,4-dihydropyridazin-3-yl]methyl}phenyl)carbamate
rac-tetrahydrofuran-3-ylmethyl(3-{[1-(1-methyl-1H-pyrazol-4-yl)-4-oxo-1,4-dihydropyridazin-3-yl]methyl}phenyl)carbamate;
(3-methyloxetan-3-yl)methyl(3-{[1-(1-methyl-1H-pyrazol-4-yl)-4-oxo-1,4-dihydropyridazin-3-yl]methyl}phenyl)carbamate;
2,2,2-trifluoroethyl(3-{[1-(1-methyl-1H-pyrazol-4-yl)-4-oxo-1,4-dihydropyridazin-3-yl]methyl}phenyl)carbamate;
3-(dimethylamino)-3-oxopropyl (3-{[1-(1-methyl-1H-pyrazol-4-yl)-4-oxo-1,4-dihydropyridazin-3-yl]methyl}phenyl)carbamate;
2-(dimethylamino)ethyl(3-{[4-oxo-1-(3,4,5-trifluorophenyl)-1,4-dihydropyridazin-3-yl]methyl}phenyl)carbamate;
2-(1H-imidazol-1-yl)ethyl(3-{[4-oxo-1-(3,4,5-trifluorophenyl)-1,4-dihydropyridazin-3-yl]methyl}phenyl)carbamate;
3-(1H-pyrrol-1-yl)propyl (3-{[4-oxo-1-(3,4,5-trifluorophenyl)-1,4-dihydropyridazin-3-yl]methyl}phenyl)carbamate;
2-(2-oxopyrrolidin-1-yl)ethyl(3-{[4-oxo-1-(3,4,5-trifluorophenyl)-1,4-dihydropyridazin-3-yl]methyl}phenyl)carbamate;
2-[methyl(phenyl)amino]ethyl(3-{[4-oxo-1-(3,4,5-trifluorophenyl)-1,4-dihydropyridazin-3-yl]methyl}phenyl)carbamate;
3-(2-oxopyrrolidin-1-yl)propyl (3-{[4-oxo-1-(3,4,5-trifluorophenyl)-1,4-dihydropyridazin-3-yl]methyl}phenyl)carbamate;
2-(1H-1,2,4-triazol-1-yl)ethyl(3-{[4-oxo-1-(3,4,5-trifluorophenyl)-1,4-dihydropyridazin-3-yl]methyl}phenyl)carbamate;
3-(4-methylpiperidin-1-yl)propyl (3-{[4-oxo-1-(3,4,5-trifluorophenyl)-1,4-dihydropyridazin-3-yl]methyl}phenyl)carbamate;
3-pyrrolidin-1-ylpropyl (3-{[4-oxo-1-(3,4,5-trifluorophenyl)-1,4-dihydropyridazin-3-yl]methyl}phenyl)carbamate;
cyclobutylmethyl(3-{[4-oxo-1-(3,4,5-trifluorophenyl)-1,4-dihydropyridazin-3-yl]methyl}phenyl)carbamate;
cyclopentylmethyl(3-{[4-oxo-1-(3,4,5-trifluorophenyl)-1,4-dihydropyridazin-3-yl]methyl}phenyl)carbamate;
cyclohexylmethyl(3-{[4-oxo-1-(3,4,5-trifluorophenyl)-1,4-dihydropyridazin-3-yl]methyl}phenyl)carbamate;
tetrahydro-2H-pyran-4-ylmethyl(3-{[4-oxo-1-(3,4,5-trifluorophenyl)-1,4-dihydropyridazin-3-yl]methyl}phenyl)carbamate;
rac-tetrahydrofuran-3-ylmethyl(3-{[4-oxo-1-(3,4,5-trifluorophenyl)-1,4-dihydropyridazin-3-yl]methyl}phenyl)carbamate;
(3-methyloxetan-3-yl)methyl(3-{[4-oxo-1-(3,4,5-trifluorophenyl)-1,4-dihydropyridazin-3-yl]methyl}phenyl)carbamate;
3-(dimethylamino)-3-oxopropyl (3-{[4-oxo-1-(3,4,5-trifluorophenyl)-1,4-dihydropyridazin-3-yl]methyl}phenyl)carbamate;
rac-tetrahydrofuran-2-ylmethyl(3-{[1-(1-methyl-1H-pyrazol-4-yl)-4-oxo-1,4-dihydropyridazin-3-yl]methyl}phenyl)carbamate;
rac-tetrahydro-2H-pyran-2-ylmethyl(3-{[1-(1-methyl-1H-pyrazol-4-yl)-4-oxo-1,4-dihydropyridazin-3-yl]methyl}phenyl)carbamate;
3,3,3-trifluoropropyl (3-{[1-(1-methyl-1H-pyrazol-4-yl)-4-oxo-1,4-dihydropyridazin-3-yl]methyl}phenyl)carbamate;
2-(tetrahydro-2H-pyran-4-yl)ethyl(3-{[1-(1-methyl-1H-pyrazol-4-yl)-4-oxo-1,4-dihydropyridazin-3-yl]methyl}phenyl)carbamate;
3-(1,1-dioxidothiomorpholin-4-yl)propyl (3-{[1-(1-methyl-1H-pyrazol-4-yl)-4-oxo-1,4-dihydropyridazin-3-yl]methyl}phenyl)carbamate;
2-(1,1-dioxidothiomorpholin-4-yl)ethyl(3-{[1-(1-methyl-1H-pyrazol-4-yl)-4-oxo-1,4-dihydropyridazin-3-yl]methyl}phenyl)carbamate;
rac-1,4-dioxan-2-ylmethyl(3-{[1-(1-methyl-1H-pyrazol-4-yl)-4-oxo-1,4-dihydropyridazin-3-yl]methyl}phenyl)carbamate;
rac-tetrahydro-2H-pyran-3-ylmethyl(3-{[1-(1-methyl-1H-pyrazol-4-yl)-4-oxo-1,4-dihydropyridazin-3-yl]methyl}phenyl)carbamate;
rac-[1-(2,2,2-trifluoro-1-methylethyl)azetidin-3-yl]methyl(3-{[1-(1-methyl-1H-pyrazol-4-yl)-4-oxo-1,4-dihydropyridazin-3-yl]methyl}phenyl)carbamate;
3-(diethylamino)propyl (3-{[1-(1-methyl-1H-pyrazol-4-yl)-4-oxo-1,4-dihydropyridazin-3-yl]methyl}phenyl)carbamate;
4-hydroxybutyl (3-{[1-(1-methyl-1H-pyrazol-4-yl)-4-oxo-1,4-dihydropyridazin-3-yl]methyl}phenyl)carbamate;
rac-2-methylbutyl (3-{[1-(1-methyl-1H-pyrazol-4-yl)-4-oxo-1,4-dihydropyridazin-3-yl]methyl}phenyl)carbamate;
(2-methylcyclopropyl)methyl(3-{[1-(1-methyl-1H-pyrazol-4-yl)-4-oxo-1,4-dihydropyridazin-3-yl]methyl}phenyl)carbamate;
3-methoxypropyl (3-{[1-(1-methyl-1H-pyrazol-4-yl)-4-oxo-1,4-dihydropyridazin-3-yl]methyl}phenyl)carbamate;
2,2-difluoroethyl(3-{[1-(1-methyl-1H-pyrazol-4-yl)-4-oxo-1,4-dihydropyridazin-3-yl]methyl}phenyl)carbamate;
2-(cyclohexyloxy)ethyl(3-{[1-(1-methyl-1H-pyrazol-4-yl)-4-oxo-1,4-dihydropyridazin-3-yl]methyl}phenyl)carbamate;
rac-oxetan-2-ylmethyl(3-{[1-(1-methyl-1H-pyrazol-4-yl)-4-oxo-1,4-dihydropyridazin-3-yl]methyl}phenyl)carbamate;
tetrahydro-2H-pyran-4-ylmethyl(3-{[1-(3-cyanophenyl)-4-oxo-1,4-dihydropyridazin-3-yl]methyl}phenyl)carbamate;
propyl (3-{[1-(3-cyanophenyl)-4-oxo-1,4-dihydropyridazin-3-yl]methyl}phenyl)carbamate;
rac-2-methoxybutyl (3-{[1-(3-cyanophenyl)-4-oxo-1,4-dihydropyridazin-3-yl]methyl}phenyl)carbamate;
2-(2-oxopyrrolidin-1-yl)ethyl(3-{[1-(3-cyanophenyl)-4-oxo-1,4-dihydropyridazin-3-yl]methyl}phenyl)carbamate;
rac-tetrahydrofuran-3-ylmethyl(3-{[1-(3-cyanophenyl)-4-oxo-1,4-dihydropyridazin-3-yl]methyl}phenyl)carbamate;
2-(3-oxomorpholin-4-yl)ethyl(3-{[1-(3-cyanophenyl)-4-oxo-1,4-dihydropyridazin-3-yl]methyl}phenyl)carbamate;
rac-[1-(2-methoxyethyl)pyrrolidin-3-yl]methyl(3-{[1-(3-cyanophenyl)-4-oxo-1,4-dihydropyridazin-3-yl]methyl}phenyl)carbamate;
2-(2,2,2-trifluoroethoxy)ethyl(3-{[1-(3-cyanophenyl)-4-oxo-1,4-dihydropyridazin-3-yl]methyl}phenyl)carbamate;
2-(1H-1,2,4-triazol-1-yl)ethyl(3-{[1-(3-cyanophenyl)-4-oxo-1,4-dihydropyridazin-3-yl]methyl}phenyl)carbamate;
3-(dimethylamino)-3-oxopropyl (3-{[1-(3-cyanophenyl)-4-oxo-1,4-dihydropyridazin-3-yl]methyl}phenyl)carbamate;
3-(dimethylamino)-3-oxopropyl (3-{[1-(3,5-difluorophenyl)-4-oxo-1,4-dihydropyridazin-3-yl]methyl}phenyl)carbamate;
propyl (3-{[1-(3,5-difluorophenyl)-4-oxo-1,4-dihydropyridazin-3-yl]methyl}phenyl)carbamate;
rac-2-methoxybutyl (3-{[1-(3,5-difluorophenyl)-4-oxo-1,4-dihydropyridazin-3-yl]methyl}phenyl)carbamate;
2-(2-oxopyrrolidin-1-yl)ethyl(3-{[1-(3,5-difluorophenyl)-4-oxo-1,4-dihydropyridazin-3-yl]methyl}phenyl)carbamate;
rac-tetrahydrofuran-3-ylmethyl(3-{[1-(3,5-difluorophenyl)-4-oxo-1,4-dihydropyridazin-3-yl]methyl}phenyl)carbamate;
rac-[1-(2-methoxyethyl)pyrrolidin-3-yl]methyl(3-{[1-(3,5-difluorophenyl)-4-oxo-1,4-dihydropyridazin-3-yl]methyl}phenyl)carbamate;
2-(2,2,2-trifluoroethoxy)ethyl(3-{[1-(3,5-difluorophenyl)-4-oxo-1,4-dihydropyridazin-3-yl]methyl}phenyl)carbamate;
2-(1,1-dioxidothiomorpholin-4-yl)ethyl(3-{[1-(3-cyanophenyl)-4-oxo-1,4-dihydropyridazin-3-yl]methyl}phenyl)carbamate;
2-morpholin-4-ylethyl(3-{[1-(3-cyanophenyl)-4-oxo-1,4-dihydropyridazin-3-yl]methyl}phenyl)carbamate;
2-(tetrahydro-2H-pyran-4-yl)ethyl(3-{[1-(3-cyanophenyl)-4-oxo-1,4-dihydropyridazin-3-yl]methyl}phenyl)carbamate;
2-(2-methoxyethoxy)ethyl(3-{[4-oxo-1-(3,4,5-trifluorophenyl)-1,4-dihydropyridazin-3-yl]methyl}phenyl)carbamate;
2-(2-methoxyethoxy)ethyl(3-{[1-(3-cyanophenyl)-4-oxo-1,4-dihydropyridazin-3-yl]methyl}phenyl)carbamate;
rac-1,4-dioxan-2-ylmethyl(3-{[1-(3-cyanophenyl)-4-oxo-1,4-dihydropyridazin-3-yl]methyl}phenyl)carbamate;
2-(2-methoxyethoxy)ethyl(3-{[1-(3,4-difluorophenyl)-4-oxo-1,4-dihydropyridazin-3-yl]methyl}phenyl)carbamate;
rac-1,4-dioxan-2-ylmethyl(3-{[1-(3,4-difluorophenyl)-4-oxo-1,4-dihydropyridazin-3-yl]methyl}phenyl)carbamate;
2-(3-oxopiperazin-1-yl)ethyl(3-{[1-(3,5-difluorophenyl)-4-oxo-1,4-dihydropyridazin-3-yl]methyl}phenyl)carbamate;
2-(3-oxomorpholin-4-yl)ethyl(3-{[1-(3,5-difluorophenyl)-4-oxo-1,4-dihydropyridazin-3-yl]methyl}phenyl)carbamate;
tetrahydro-2H-pyran-4-ylmethyl(3-{[1-(3,5-difluorophenyl)-4-oxo-1,4-dihydropyridazin-3-yl]methyl}phenyl)carbamate;
rac-tetrahydro-2H-pyran-3-ylmethyl(3-{[1-(3,5-difluorophenyl)-4-oxo-1,4-dihydropyridazin-3-yl]methyl}phenyl)carbamate;
2-(2-methoxyethoxy)ethyl(3-{[1-(3,5-difluorophenyl)-4-oxo-1,4-dihydropyridazin-3-yl]methyl}phenyl)carbamate;
(2,2,6,6-tetramethyltetrahydro-2H-pyran-4-yl)methyl(3-{[1-(1-methyl-1H-pyrazol-4-yl)-4-oxo-1,4-dihydropyridazin-3-yl]methyl}phenyl)carbamate;
(2S)-2-[(2R or S)-2-methyl-5-oxopyrrolidin-1-yl]propyl (3-{[1-(1-methyl-1H-pyrazol-4-yl)-4-oxo-1,4-dihydropyridazin-3-yl]methyl}phenyl)carbamate;
rac-(2,2-dimethyltetrahydro-2H-pyran-4-yl)methyl(3-{[1-(1-methyl-1H-pyrazol-4-yl)-4-oxo-1,4-dihydropyridazin-3-yl]methyl}phenyl)carbamate;
rac-2-(4-hydroxy-2,2-dimethyltetrahydro-2H-pyran-4-yl)ethyl(3-{[1-(1-methyl-1H-pyrazol-4-yl)-4-oxo-1,4-dihydropyridazin-3-yl]methyl}phenyl)carbamate;
(4-fluorotetrahydro-2H-pyran-4-yl)methyl(3-{[1-(1-methyl-1H-pyrazol-4-yl)-4-oxo-1,4-dihydropyridazin-3-yl]methyl}phenyl)carbamate;
3-amino-2,2-difluoropropyl (3-{[1-(1-methyl-1H-pyrazol-4-yl)-4-oxo-1,4-dihydropyridazin-3-yl]methyl}phenyl)carbamate;
(2R)-pyrrolidin-2-ylmethyl(3-{[1-(1-methyl-1H-pyrazol-4-yl)-4-oxo-1,4-dihydropyridazin-3-yl]methyl}phenyl)carbamate;
(2S)-pyrrolidin-2-ylmethyl(3-{[1-(1-methyl-1H-pyrazol-4-yl)-4-oxo-1,4-dihydropyridazin-3-yl]methyl}phenyl)carbamate;
piperidin-4-ylmethyl(3-{[1-(1-methyl-1H-pyrazol-4-yl)-4-oxo-1,4-dihydropyridazin-3-yl]methyl}phenyl)carbamate;
piperidin-4-yl (3-{[1-(1-methyl-1H-pyrazol-4-yl)-4-oxo-1,4-dihydropyridazin-3-yl]methyl}phenyl)carbamate;
rac-2-amino-3,3,3-trifluoropropyl (3-{[1-(1-methyl-1H-pyrazol-4-yl)-4-oxo-1,4-dihydropyridazin-3-yl]methyl}phenyl)carbamate;
(4-fluoropiperidin-4-yl)methyl(3-{[1-(1-methyl-1H-pyrazol-4-yl)-4-oxo-1,4-dihydropyridazin-3-yl]methyl}phenyl)carbamate;
rac-3-amino-2-fluoropropyl (3-{[1-(1-methyl-1H-pyrazol-4-yl)-4-oxo-1,4-dihydropyridazin-3-yl]methyl}phenyl)carbamate;
2-(methylamino)ethyl(3-{[4-oxo-1-(3,4,5-trifluorophenyl)-1,4-dihydropyridazin-3-yl]methyl}phenyl)carbamate;
3-piperazin-1-ylpropyl (3-{[4-oxo-1-(3,4,5-trifluorophenyl)-1,4-dihydropyridazin-3-yl]methyl}phenyl)carbamate;
2-piperidin-4-ylethyl(3-{[1-(1-methyl-1H-pyrazol-4-yl)-4-oxo-1,4-dihydropyridazin-3-yl]methyl}phenyl)carbamate;
2-piperazin-1-ylethyl(3-{[1-(1-methyl-1H-pyrazol-4-yl)-4-oxo-1,4-dihydropyridazin-3-yl]methyl}phenyl)carbamate;
3-piperazin-1-ylpropyl (3-{[1-(1-methyl-1H-pyrazol-4-yl)-4-oxo-1,4-dihydropyridazin-3-yl]methyl}phenyl)carbamate;
azetidin-3-ylmethyl(3-{[1-(3-cyanophenyl)-4-oxo-1,4-dihydropyridazin-3-yl]methyl}phenyl)carbamate;
2-piperazin-1-ylethyl(3-{[1-(3,5-difluorophenyl)-4-oxo-1,4-dihydropyridazin-3-yl]methyl}phenyl)carbamate;
({[(3-{[1-(1-methyl-1H-pyrazol-4-yl)-4-oxo-1,4-dihydropyridazin-3-yl]methyl}phenyl)amino]carbonyl}oxy)acetic acid;
2-hydroxyethyl(3-{[1-(1-methyl-1H-pyrazol-4-yl)-4-oxo-1,4-dihydropyridazin-3-yl]methyl}phenyl)carbamate;
N-{3-[(4-Oxo-1-phenyl-1,4-dihydropyridazin-3-yl)methyl]phenyl}acetamide;
N-(3-{[1-(1-methyl-1H-pyrazol-4-yl)-4-oxo-1,4-dihydropyridazin-3-yl]methyl}phenyl)-2-phenylacetamide;
N-(3-{[1-(1-methyl-1H-pyrazol-4-yl)-4-oxo-1,4-dihydropyridazin-3-yl]methyl}phenyl)-3-phenylpropanamide;
N-(3-{[1-(1-methyl-1H-pyrazol-4-yl)-4-oxo-1,4-dihydropyridazin-3-yl]methyl}phenyl)butanamide;
N-(3-{[1-(1-methyl-1H-pyrazol-4-yl)-4-oxo-1,4-dihydropyridazin-3-yl]methyl}phenyl)propanamide;
1-(1-methyl-1H-pyrazol-4-yl)-3-[3-(2-oxopyrrolidin-1-yl)benzyl]pyridazin-4(1H)-one;
1-(1-methyl-1H-pyrazol-4-yl)-3-[3-(2-oxo-1,3-oxazolidin-3-yl)benzyl]pyridazin-4(1H)-one;
rac-2-fluoro-3-morpholin-4-ylpropyl (3-{[1-(1-methyl-1H-pyrazol-4-yl)-4-oxo-1,4-dihydropyridazin-3-yl]methyl}phenyl)carbamate;
rac-ethyl(3-{fluoro[1-(1-methyl-1H-pyrazol-4-yl)-4-oxo-1,4-dihydropyridazin-3-yl]methyl}phenyl)carbamate;
rac-ethyl{3-[fluoro(4-oxo-1-phenyl-1,4-dihydropyridazin-3-yl)methyl]phenyl}carbamate;
rac-ethyl{3-[[1-(4-chlorophenyl)-4-oxo-1,4-dihydropyridazin-3-yl](fluoro)methyl]phenyl}carbamate;
ethyl {3-[(4-oxo-1-phenyl-1,4-dihydropyridazin-3-yl)methyl]phenyl}carbamate;
ethyl [3-({1-[1-(2-hydroxyethyl)-1H-pyrazol-4-yl]-4-oxo-1,4-dihydropyridazin-3-yl}methyl)phenyl]carbamate;
propyl [3-({1-[1-(2-hydroxyethyl)-1H-pyrazol-4-yl]-4-oxo-1,4-dihydropyridazin-3-yl}methyl)phenyl]carbamate;
2-methylpropyl[3-({1-[1-(2-hydroxyethyl)-1H-pyrazol-4-yl]-4-oxo-1,4-dihydropyridazin-3-yl}methyl)phenyl]carbamate;
ethyl(3-{[1-(3-hydroxyphenyl)-4-oxo-1,4-dihydropyridazin-3-yl]methyl}phenyl)carbamate ethyl [3-({1-[1-(2-aminoethyl)-1H-pyrazol-4-yl]-4-oxo-1,4-dihydropyridazin-3-yl}methyl)phenyl]carbamate;
2-methylpropyl[3-({1-[1-(2-aminoethyl)-1H-pyrazol-4-yl]-4-oxo-1,4-dihydropyridazin-3-yl}methyl)phenyl]carbamate;
ethyl(3-{[1-(3-methoxyphenyl)-4-oxo-1,4-dihydropyridazin-3-yl]methyl}phenyl)carbamate;
ethyl 3-((1-(3-ethoxyphenyl)-4-oxo-1,4-dihydropyridazin-3-yl)methyl)phenylcarbamate;
rac-ethyl{3-[1-(4-oxo-1-phenyl-1,4-dihydropyridazin-3-yl)ethyl]phenyl}carbamate;
2-methoxyethyl(3-{(1S or R)-1-[1-(1-methyl-1H-pyrazol-4-yl)-4-oxo-1,4-dihydropyridazin-3-yl]ethyl}phenyl)carbamate;
2-methoxyethyl(3-{(1R or S)-1-[1-(1-methyl-1H-pyrazol-4-yl)-4-oxo-1,4-dihydropyridazin-3-yl]ethyl}phenyl)carbamate;
rac-3-[[3-(5-ethoxypyrimidin-2-yl)phenyl](hydroxy)methyl]-1-(1-methyl-1H-pyrazol-4-yl)pyridazin-4(1H)-one;
3-[(S or R)-[3-(5-ethoxypyrimidin-2-yl)phenyl](fluoro)methyl]-1-(1-methyl-1H-pyrazol-4-yl)pyridazin-4(1H)-one;
3-[(R or S)-[3-(5-ethoxypyrimidin-2-yl)phenyl](fluoro)methyl]-1-(1-methyl-1H-pyrazol-4-yl)pyridazin-4(1H)-one;
rac-3-[[3-(5-ethoxypyrimidin-2-yl)phenyl](fluoro)methyl]-1-(1-methyl-1H-pyrazol-4-yl)pyridazin-4(1H)-one-d3;
rac-3-[[3-(5-ethoxypyrimidin-2-yl)phenyl](fluoro)methyl]-1-(1-methyl-1H-pyrazol-4-yl)pyridazin-4(1H)-one-d8;
rac-3-[[3-(5-ethoxypyrimidin-2-yl)phenyl](methoxy)methyl]-1-(1-methyl-1H-pyrazol-4-yl)pyridazin-4(1H)-one;
3-[3-(5-ethoxypyrimidin-2-yl)benzyl]-1-[1-(oxetan-3-yl)-1H-pyrazol-4-yl]pyridazin-4(1H)-one;
tert-butyl 3-(4-{3-[3-(5-ethoxypyrimidin-2-yl)benzyl]-4-oxopyridazin-1(4H)-yl}-1H-pyrazol-1-yl)azetidine-1-carboxylate;
2-(4-{3-[3-(5-ethoxypyrimidin-2-yl)benzyl]-4-oxopyridazin-1(4H)-yl}-1H-pyrazol-1-yl)-N,N-dimethylacetamide;
1-(1-azetidin-3-yl-1H-pyrazol-4-yl)-3-[3-(5-ethoxypyrimidin-2-yl)benzyl]pyridazin-4(1H)-one;
3-[3-(5-ethoxypyrimidin-2-yl)benzyl]-1-(1-piperidin-4-yl-1H-pyrazol-4-yl)pyridazin-4(1H)-one;
rac-4-[3-(5-ethoxypyrimidin-2-yl)phenyl]-4-[4-oxo-1-(1H-pyrazol-4-yl)-1,4-dihydropyridazin-3-yl]butanenitrile;
3-[3-(5-ethoxypyrimidin-2-yl)benzyl]-1-[1-(2-hydroxy-2-methylpropyl)-1H-pyrazol-4-yl]pyridazin-4(1H)-one;
2-methylpropyl {3-[(1-{1-[3-(dimethylamino)propyl]-1H-pyrazol-4-yl}-4-oxo-1,4-dihydropyridazin-3-yl)methyl]phenyl}carbamate;
3-[3-(5-ethoxypyrimidin-2-yl)benzyl]-1-[1-(1-methylazetidin-3-yl)-1H-pyrazol-4-yl]pyridazin-4(1H)-one;
1-phenyl-3-[3-(pyrimidin-2-yl)benzyl]pyridazin-4(1H)-one;
3-[3-(1-methyl-1H-1,2,4-triazol-3-yl)benzyl]-1-phenylpyridazin-4(1H)-one;
1-phenyl-3-(3-pyridin-2-ylbenzyl)pyridazin-4(1H)-one;
3-[3-(5-methyl-1H-imidazol-2-yl)benzyl]-1-(1-methyl-1H-pyrazol-4-yl)pyridazin-4(1H)-one;
3-[3-(1-methyl-1H-imidazol-4-yl)benzyl]-1-(1-methyl-1H-pyrazol-4-yl)pyridazin-4(1H)-one;
ethyl 2-(3-{[1-(1-methyl-1H-pyrazol-4-yl)-4-oxo-1,4-dihydropyridazin-3-yl]methyl}phenyl)-1,3-oxazole-4-carboxylate;
ethyl 2-(3-{[1-(1-methyl-1H-pyrazol-4-yl)-4-oxo-1,4-dihydropyridazin-3-yl]methyl}phenyl)-1,3-oxazole-5-carboxylate;
3-{3-[5-(hydroxymethyl)-1,3-thiazol-2-yl]benzyl}-1-(1-methyl-1H-pyrazol-4-yl)pyridazin-4(1H)-one;
3-[3-(5-ethoxypyridin-2-yl)benzyl]-1-(1-methyl-1H-pyrazol-4-yl)pyridazin-4(1H)-one;
rac-3-{1-[3-(4-ethoxypyrimidin-2-yl)phenyl]ethyl}-1-(1-methyl-1H-pyrazol-4-yl)pyridazin-4(1H)-one;
rac-3-(1-{3-[4-(2-methoxyethoxy)pyrimidin-2-yl]phenyl}ethyl)-1-(1-methyl-1H-pyrazol-4-yl)pyridazin-4(1H)-one;
3-{(1S or R)-1-[3-(1-ethyl-1H-1,2,4-triazol-3-yl)phenyl]ethyl}-1-(1-methyl-1H-pyrazol-4-yl)pyridazin-4(1H)-one;
3-{(1R or S)-1-[3-(1-ethyl-1H-1,2,4-triazol-3-yl)phenyl]ethyl}-1-(1-methyl-1H-pyrazol-4-yl)pyridazin-4(1H)-one;
1-(1-methyl-1H-pyrazol-4-yl)-3-{(1S or R)-1-[3-(1-methyl-1H-1,2,4-triazol-3-yl)phenyl]ethyl}pyridazin-4(1H)-one;
1-(1-methyl-1H-pyrazol-4-yl)-3-{(1R or S)-1-[3-(1-methyl-1H-1,2,4-triazol-3-yl)phenyl]ethyl}pyridazin-4(1H)-one;
1-(1-methyl-1H-pyrazol-4-yl)-3-{(1S or R)-1-[3-(1-propyl-1H-1,2,4-triazol-3-yl)phenyl]ethyl}pyridazin-4(1H)-one;
1-(1-methyl-1H-pyrazol-4-yl)-3-{(1R or S)-1-[3-(1-propyl-1H-1,2,4-triazol-3-yl)phenyl]ethyl}pyridazin-4(1H)-one;
1-(1-ethyl-1H-pyrazol-4-yl)-3-{(1S or R)-1-[3-(1-ethyl-1H-1,2,4-triazol-3-yl)phenyl]ethyl}pyridazin-4(1H)-one;
1-(1-ethyl-1H-pyrazol-4-yl)-3-{(1R or S)-1-[3-(1-ethyl-1H-1,2,4-triazol-3-yl)phenyl]ethyl}pyridazin-4(1H)-one;
1-(1-ethyl-1H-pyrazol-4-yl)-3-{(1S or R)-1-[3-(1-propyl-1H-1,2,4-triazol-3-yl)phenyl]ethyl}pyridazin-4(1H)-one;
1-(1-ethyl-1H-pyrazol-4-yl)-3-{(1R or S)-1-[3-(1-propyl-1H-1,2,4-triazol-3-yl)phenyl]ethyl}pyridazin-4(1H)-one;
3-[(1R or S)-1-{3-[4-(difluoromethyl)pyrimidin-2-yl]phenyl}ethyl]-1-(1-methyl-1H-pyrazol-4-yl)pyridazin-4(1H)-one;
1-(1-methyl-1H-pyrazol-4-yl)-3-{(1R or S)-1-[3-(4-methylpyrimidin-2-yl)phenyl]ethyl}pyridazin-4(1H)-one;
3-{(1R or S)-1-[3-(4-cyclopropyl-5-fluoropyrimidin-2-yl)phenyl]ethyl}-1-(1-methyl-1H-pyrazol-4-yl)pyridazin-4(1H)-one;
3-{(1R or S)-1-[3-(5-fluoro-4-methylpyrimidin-2-yl)phenyl]ethyl}-1-(1-methyl-1H-pyrazol-4-yl)pyridazin-4(1H)-one;
3-[(1R or S)-1-{3-[4-(2-hydroxypropan-2-yl)pyrimidin-2-yl]phenyl}ethyl]-1-(1-methyl-1H-pyrazol-4-yl)pyridazin-4(1H)-one;
3-[(1R or S)-1-(3-{4-[(2-methoxyethyl)amino]pyrimidin-2-yl}phenyl)ethyl]-1-(1-methyl-1H-pyrazol-4-yl)pyridazin-4(1H)-one;
2-methylpropyl {3-[(1-{1-[4-(dimethylamino)-4-oxobutyl]-1H-pyrazol-4-yl}-4-oxo-1,4-dihydropyridazin-3-yl)methyl]phenyl}carbamate;
2-methylpropyl {3-[(1-{1-[4-(methylamino)-4-oxobutyl]-1H-pyrazol-4-yl}-4-oxo-1,4-dihydropyridazin-3-yl)methyl]phenyl}carbamate;
2-methylpropyl[3-({1-[1-(4-morpholin-4-yl-4-oxobutyl)-1H-pyrazol-4-yl]-4-oxo-1,4-dihydropyridazin-3-yl}methyl)phenyl]carbamate;
2-methylpropyl[3-({4-oxo-1-[1-(4-oxo-4-piperidin-1-ylbutyl)-1H-pyrazol-4-yl]-1,4-dihydropyridazin-3-yl}methyl)phenyl]carbamate;
2-methylpropyl[3-({4-oxo-1-[1-(4-oxo-4-pyrrolidin-1-ylbutyl)-1H-pyrazol-4-yl]-1,4-dihydropyridazin-3-yl}methyl)phenyl]carbamate;
2-methylpropyl {3-[(1-{1-[4-(oxetan-3-ylamino)-4-oxobutyl]-1H-pyrazol-4-yl}-4-oxo-1,4-dihydropyridazin-3-yl)methyl]phenyl}carbamate;
2-methylpropyl {3-[(1-{1-[3-(methylamino)-3-oxopropyl]-1H-pyrazol-4-yl}-4-oxo-1,4-dihydropyridazin-3-yl)methyl]phenyl}carbamate;
2-methylpropyl[3-({1-[1-(3-morpholin-4-yl-3-oxopropyl)-1H-pyrazol-4-yl]-4-oxo-1,4-dihydropyridazin-3-yl}methyl)phenyl]carbamate;
2-methylpropyl {3-[(1-{1-[3-(oxetan-3-ylamino)-3-oxopropyl]-1H-pyrazol-4-yl}-4-oxo-1,4-dihydropyridazin-3-yl)methyl]phenyl}carbamate;
2-methylpropyl (3-{[1-(1-{4-[(2-hydroxy-2-methylpropyl)amino]-4-oxobutyl}-1H-pyrazol-4-yl)-4-oxo-1,4-dihydropyridazin-3-yl]methyl}phenyl)carbamate;
rac-2-methylpropyl (3-{[1-(1-{4-[(2-hydroxypropyl)amino]-4-oxobutyl}-1H-pyrazol-4-yl)-4-oxo-1,4-dihydropyridazin-3-yl]methyl}phenyl)carbamate;
1-(1-methyl-1H-pyrazol-4-yl)-3-{3-[1-(oxetan-3-yl)-1H-1,2,4-triazol-3-yl]benzyl}pyridazin-4(1H)-one;
3-[3-(1-ethyl-1H-1,2,4-triazol-3-yl)benzyl]-1-(1-methyl-1H-pyrazol-4-yl)pyridazin-4(1H)-one;
3-[3-(3-{[1-(1-methyl-1H-pyrazol-4-yl)-4-oxo-1,4-dihydropyridazin-3-yl]methyl}phenyl)-1H-1,2,4-triazol-1-yl]propanenitrile;
N,N-dimethyl-3-[3-(3-{[1-(1-methyl-1H-pyrazol-4-yl)-4-oxo-1,4-dihydropyridazin-3-yl]methyl}phenyl)-1H-1,2,4-triazol-1-yl]propanamide;
rac-1-(1-methyl-1H-pyrazol-4-yl)-3-(3-{1-[2-(tetrahydrofuran-2-yl)ethyl]-1H-1,2,4-triazol-3-yl}benzyl)pyridazin-4(1H)-one;
3-{3-[1-(2,2-difluoro-3-morpholin-4-ylpropyl)-1H-1,2,4-triazol-3-yl]benzyl}-1-(1-methyl-1H-pyrazol-4-yl)pyridazin-4(1H)-one;
3-(3-{1-[(3-methyloxetan-3-yl)methyl]-1H-1,2,4-triazol-3-yl}benzyl)-1-(1-methyl-1H-pyrazol-4-yl)pyridazin-4(1H)-one;
1-(1-methyl-1H-pyrazol-4-yl)-3-(3-{1-[2-(methylsulfonyl)ethyl]-1H-1,2,4-triazol-3-yl}benzyl)pyridazin-4(1H)-one;
1-(1-methyl-1H-pyrazol-4-yl)-3-{3-[1-(2-phenylethyl)-1H-1,2,4-triazol-3-yl]benzyl}pyridazin-4(1H)-one;
3-{3-[1-(2-ethoxyethyl)-1H-1,2,4-triazol-3-yl]benzyl}-1-(1-methyl-1H-pyrazol-4-yl)pyridazin-4(1H)-one;
rac-1-(1-methyl-1H-pyrazol-4-yl)-3-(3-{1-[2-(tetrahydrofuran-3-yl)ethyl]-1H-1,2,4-triazol-3-yl}benzyl)pyridazin-4(1H)-one;
1-(1-methyl-1H-pyrazol-4-yl)-3-(3-{1-[2-(2-oxopyrrolidin-1-yl)ethyl]-1H-1,2,4-triazol-3-yl}benzyl)pyridazin-4(1H)-one;
3-{3-[1-(2-methoxy-2-methylpropyl)-1H-1,2,4-triazol-3-yl]benzyl}-1-(1-methyl-1H-pyrazol-4-yl)pyridazin-4(1H)-one;
3-(3-{1-[3-(5,5-dimethyl-1,3-dioxan-2-yl)propyl]-1H-1,2,4-triazol-3-yl}benzyl)-1-(1-methyl-1H-pyrazol-4-yl)pyridazin-4(1H)-one;
rac-1-(1-methyl-1H-pyrazol-4-yl)-3-(3-{1-[3-(tetrahydrofuran-2-yl)propyl]-1H-1,2,4-triazol-3-yl}benzyl)pyridazin-4(1H)-one;
rac-4-{3-[3-(5-ethoxypyrimidin-2-yl)benzyl]-4-oxopyridazin-1(4H)-yl}-2-fluoro-N-(2-hydroxyethyl)benzamide;
4-{3-[3-(5-ethoxypyrimidin-2-yl)benzyl]-4-oxopyridazin-1(4H)-yl}-2-fluoro-N-methylbenzamide;
4-{3-[3-(5-ethoxypyrimidin-2-yl)benzyl]-4-oxopyridazin-1(4H)-yl}-2-fluoro-N-(2-hydroxy-2-methylpropyl)benzamide;
3-(3-(5-ethoxypyrimidin-2-yl)benzyl)-1-(3-fluorophenyl)pyridazin-4(1H)-one;
3-{3-[3-(5-ethoxypyrimidin-2-yl)benzyl]-4-oxopyridazin-1(4H)-yl}-N,N-dimethylbenzamide;
2-methylpropyl[3-({4-oxo-1-[4-(pyridin-3-yl)phenyl]-1,4-dihydropyridazin-3-yl}methyl)phenyl]carbamate;
2-methylpropyl (3-{[4-oxo-1-(4-pyridin-4-ylphenyl)-1,4-dihydropyridazin-3-yl]methyl}phenyl)carbamate;
2-methylpropyl[3-({1-[3-(1-methyl-1H-pyrazol-4-yl)phenyl]-4-oxo-1,4-dihydropyridazin-3-yl}methyl)phenyl]carbamate;
2-methylpropyl (3-{[4-oxo-1-(3-pyridin-4-ylphenyl)-1,4-dihydropyridazin-3-yl]methyl}phenyl)carbamate;
2-methylpropyl[3-({4-oxo-1-[4-(1H-pyrazol-4-yl)phenyl]-1,4-dihydropyridazin-3-yl}methyl)phenyl]carbamate;
2-methylpropyl[3-({1-[4-(1-methyl-1H-pyrazol-4-yl)phenyl]-4-oxo-1,4-dihydropyridazin-3-yl}methyl)phenyl]carbamate;
5-{3-[3-(5-methoxypyrimidin-2-yl)benzyl]-4-oxopyridazin-1(4H)-yl}pyridine-3-carbonitrile;
3-{3-[3-(1-ethyl-1H-1,2,4-triazol-3-yl)benzyl]-4-oxopyridazin-1(4H)-yl}benzamide;
3-{4-oxo-3-[3-(1-propyl-1H-1,2,4-triazol-3-yl)benzyl]pyridazin-1(4H)-yl}benzamide;
3-[3-[3-(5-ethoxypyrimidin-2-yl)benzyl]-4-oxopyridazin-1(4H)-yl]benzamide;
2-methoxyethyl [3-({1-[3-(aminocarbonyl)-5-fluorophenyl]-4-oxo-1,4-dihydropyridazin-3-yl}methyl)phenyl]carbamate;
rac-3-[3-{1-[3-(5-ethoxypyrimidin-2-yl)phenyl]ethyl}-4-oxopyridazin-1(4H)-yl]benzamide;
3-[3-(5-ethyl-1,2,4-oxadiazole-3-yl)benzyl]-1-(1-methyl-1H-pyrazol-4-yl)pyridazin-4(1H)-one;
1-(1-methyl-1H-pyrazol-4-yl)-3-[3-(5-propyl-1,2,4-oxadiazol-3-yl)benzyl]pyridazin-4(1H)-one;
3-[3-(5-butyl-1,2,4-oxadiazol-3-yl)benzyl]-1-(1-methyl-1H-pyrazol-4-yl)pyridazin-4(1H)-one;
3-{3-[5-(2-methylpropyl)-1,2,4-oxadiazol-3-yl]benzyl}-1-(1-methyl-1H-pyrazol-4-yl)pyridazin-4(1H)-one;
1-(1-methyl-1H-pyrazol-4-yl)-3-{3-[5-(morpholin-4-ylmethyl)-1,2,4-oxadiazol-3-yl]benzyl}pyridazin-4(1H)-one;
3-{3-[5-(2-methoxyethyl)-1,2,4-oxadiazol-3-yl]benzyl}-1-(1-methyl-1H-pyrazol-4-yl)pyridazin-4(1H)-one;
1-(1-methyl-1H-pyrazol-4-yl)-3-{3-[5-(oxetan-3-ylmethyl)-1,2,4-oxadiazol-3-yl]benzyl}pyridazin-4(1H)-one;
1-(1-methyl-1H-pyrazol-4-yl)-3-{3-[5-(tetrahydro-2H-pyran-4-ylmethyl)-1,2,4-oxadiazol-3-yl]benzyl}pyridazin-4(1H)-one;
3-(3-{5-[(trans-4-hydroxycyclohexyl)oxy]pyrimidin-2-yl}benzyl)-1-(1-methyl-1H-pyrazol-4-yl)pyridazin-4(1H)-one;
3-(3-{5-[(cis-4-hydroxycyclohexyl)oxy]pyrimidin-2-yl}benzyl)-1-(1-methyl-1H-pyrazol-4-yl)pyridazin-4(1H)-one;
rac-3-{3-[5-(trans-4-fluoro-3-hydroxypiperidin-4-yl)pyrimidin-2-yl]benzyl}-1-(1-methyl-1H-pyrazol-4-yl)pyridazin-4(1H)-one;
3-{3-[5-(2-methoxyethoxy)pyrimidin-2-yl]benzyl}-5-methyl-1-(1-methyl-1H-pyrazol-4-yl)pyridazin-4(1H)-one;
5-fluoro-1-(5-fluoro-1-methyl-1H-pyrazol-4-yl)-3-{3-[5-(2-methoxyethoxy)pyrimidin-2-yl]benzyl}pyridazin-4(1H)-one;
3-[3-(5-ethoxypyrimidin-2-yl)benzyl]-5-fluoro-1-(5-fluoro-1-methyl-1H-pyrazol-4-yl)pyridazin-4(1H)-one;
rac-3-[fluoro(quinolin-6-yl)methyl]-1-(1-methyl-1H-pyrazol-4-yl)pyridazin-4(1H)-one;
1-(4-chlorophenyl)-3-(4-hydroxybenzyl)pyridazin-4(1H)-one;
3-[3-(5-ethoxypyrimidin-2-yl)phenoxy]-1-(1-methyl-1H-pyrazol-4-yl)pyridazin-4(1H)-one;
3-{[3-(5-ethoxypyrimidin-2-yl)phenyl]sulfanyl}-1-(1-methyl-1H-pyrazol-4-yl)pyridazin-4(1H)-one;
or a pharmaceutically acceptable salt thereof.

8. The compound of claim 7 selected from:
3-[3-(5-ethoxypyrimidin-2-yl)benzyl]-1-(1-methyl-1H-pyrazol-4-yl)pyridazin-4(1H)-one;
3-{3-[5-(2-methoxyethoxy)pyrimidin-2-yl]benzyl}-1-(1-methyl-1H-pyrazol-4-yl)pyridazin-4(1H)-one;
ethyl(3-{[1-(1-methyl-1H-pyrazol-4-yl)-4-oxo-1,4-dihydropyridazin-3-yl]methyl}phenyl)carbamate;
3-{3-[5-(2-hydroxy-2-methylpropoxy)pyrimidin-2-yl]benzyl}-1-(1-methyl-1H-pyrazol-4-yl)pyridazin-4(1H)-one;
tetrahydrofuran-2-ylmethyl(3-{[1-(1-methyl-1H-pyrazol-4-yl)-4-oxo-1,4-dihydropyridazin-3-yl]methyl}phenyl)carbamate;
tetrahydro-2H-pyran-3-ylmethyl(3-{[1-(1-methyl-1H-pyrazol-4-yl)-4-oxo-1,4-dihydropyridazin-3-yl]methyl}phenyl)carbamate;
3-methoxypropyl (3-{[1-(1-methyl-1H-pyrazol-4-yl)-4-oxo-1,4-dihydropyridazin-3-yl]methyl}phenyl)carbamate;
2-methoxyethyl(3-{[1-(1-methyl-1H-pyrazol-4-yl)-4-oxo-1,4-dihydropyridazin-3-yl]methyl}phenyl)carbamate;
propyl (3-{[1-(1-methyl-1H-pyrazol-4-yl)-4-oxo-1,4-dihydropyridazin-3-yl]methyl}phenyl)carbamate;
2-methoxyethyl(3-{[1-(3,4-difluorophenyl)-4-oxo-1,4-dihydropyridazin-3-yl]methyl}phenyl)carbamate;
tetrahydrofuran-3-ylmethyl(3-{[1-(1-methyl-1H-pyrazol-4-yl)-4-oxo-1,4-dihydropyridazin-3-yl]methyl}phenyl)carbamate;
1-(1-methyl-1H-pyrazol-4-yl)-3-{3-[5-(tetrahydrofuran-2-ylmethoxy)pyrimidin-2-yl]benzyl}pyridazin-4(1H)-one;
2-methylpropyl[3-({1-[1-(2-hydroxyethyl)-1H-pyrazol-4-yl]-4-oxo-1,4-dihydropyridazin-3-yl}methyl)phenyl]carbamate;
3-fluoro-5-[3-{3-[5-(2-hydroxy-2-methylpropoxy)pyrimidin-2-yl]benzyl}-4-oxopyridazin-1(4H)-yl]benzonitrile;
3-{(1R or S)-1-[3-(5-methoxypyrimidin-2-yl)phenyl]ethyl}-1-(1-methyl-1H-pyrazol-4-yl)pyridazin-4(1H)-one;
3-((1R or S)-1-{3-[5-(2-hydroxy-2-methylpropoxy)pyrimidin-2-yl]phenyl}ethyl)-1-(1-methyl-1H-pyrazol-4-yl)pyridazin-4(1H)-one;
or a pharmaceutically acceptable salt thereof.

9. A pharmaceutical composition that is comprised of a compound in accordance with claim 1 and a pharmaceutically acceptable carrier.

10. A method of treating or preventing cancer in a mammal in need of such treatment that is comprised of administering to said mammal a therapeutically effective amount of a compound of claim 1.

11. A compound which is: 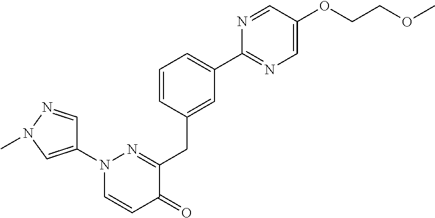
3-{3-[5-(2-Methoxyethoxy)pyrimidin-2-yl]benzyl}-1-(1-methyl-1H-pyrazol-4-yl)pyridazin-4(1H)-one
or a pharmaceutically acceptable salt thereof.

12. A compound which is: 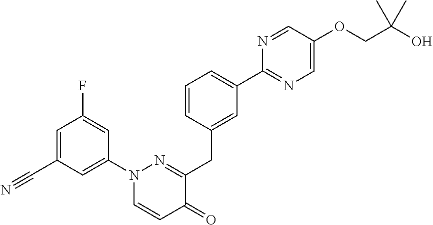
3-Fluoro-5-[3-{3-[5-(2-hydroxy-2-methylpropoxy)pyrimidin-2-yl]benzyl}-4-oxopyridazin-1(4H)-yl]benzonitrile
or a pharmaceutically acceptable salt thereof.

13. A compound which is: 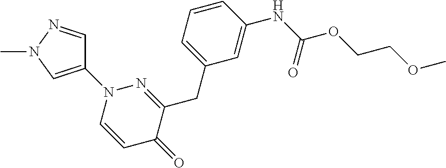
2-methoxyethyl(3-{[1-(1-methyl-1H-pyrazol-4-yl)-4-oxo-1,4-dihydropyridazin-3-yl]methyl}phenyl)carbamate
or a pharmaceutically acceptable salt thereof.

14. A compound which is: 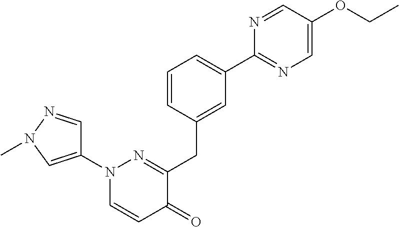
3-[3-(5-Ethoxypyrimidin-2-yl)benzyl]-1-(1-methyl-1H-pyrazol-4-yl)pyridazin-4(1H)-one
or a pharmaceutically acceptable salt thereof.

15. A compound which is: 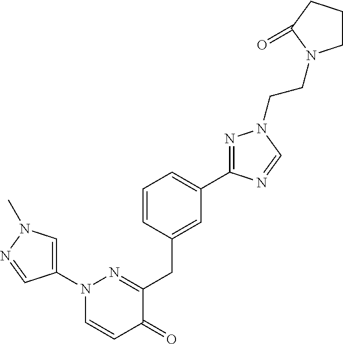
1-(1-methyl-1H-pyrazol-4-yl)-3-(3-{1-[2-(2-oxopyrrolidin-1-yl)ethyl]-1H-1,2,4-triazol-3-yl}benzyl)pyridazin-4(1H)-one
or a pharmaceutically acceptable salt thereof.

16. A compound which is: 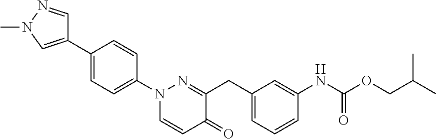
2-methylpropyl[3-({1-[4-(1-methyl-1H-pyrazol-4-yl)phenyl]-4-oxo-1,4-dihydropyridazin-3-yl}methyl)phenyl]carbamate
or a pharmaceutically acceptable salt thereof.

17. A compound which is: 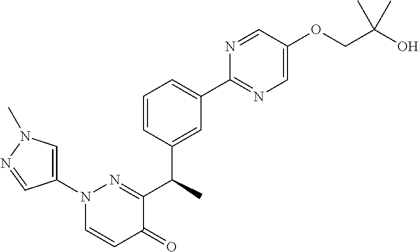
3-((1R)-1-{3-[5-(2-Hydroxy-2-methylpropoxy)pyrimidin-2-yl]phenyl}ethyl)-1-(1-methyl-1H-pyrazol-4-yl)pyridazin-4(1H)-one
or a pharmaceutically acceptable salt thereof.

18. A compound which is: 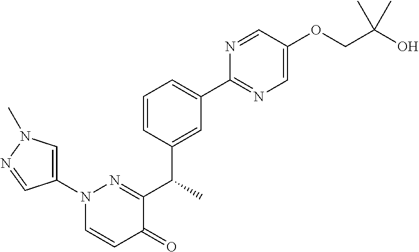
3-((1S)-1-{3-[5-(2-Hydroxy-2-methylpropoxy)pyrimidin-2-yl]phenyl}ethyl)-1-(1-methyl-1H-pyrazol-4-yl)pyridazin-4(1H)-one
or a pharmaceutically acceptable salt thereof.
